(19) United States
(12) Patent Application Publication
(10) Pub. No.: US 2009/0317834 A1
(43) Pub. Date: Dec. 24, 2009

(54) NOVEL CELLULAR GLYCAN COMPOSITIONS

(76) Inventors: Jarmo Laine, Helsinki (FI); Tero Satomaa, Helsinki (FI); Jari Natunen, Vantaa (FI); Annamari Heiskanen, Helsinki (FI); Juhani Saarinen, Helsinki (FI); Taina Jaatinen, Helsinki (FI); Milla Mikkola, Helsinki (FI); Suvi Natunen, Vantaa (FI)

Correspondence Address: BIRCH STEWART KOLASCH & BIRCH, PO BOX 747, FALLS CHURCH, VA, 22040-0747, US

(21) Appl. No.: 12/306,774

(22) PCT Filed: Jun. 29, 2007
(86) PCT No.: PCT/FI07/50405

§ 371 Date: Aug. 10, 2009

(30) Foreign Application Priority Data
Mar. 8, 2006 (FI) 20070200
Jun. 29, 2006 (FI) 20060630
May 10, 2007 (FI) 20070369

(51) Int. Cl. G01N 33/567; C12N 5/06

(52) U.S. Cl. 435/7.21; 435/325

(57) ABSTRACT

The invention describes novel compositions of glycans, glycomes, from human embryonic stem cells, and especially novel subcompositions of the glycomes with specific monosaccharide compositions and glycan structures. The invention is further directed to methods for modifying the glycomes and analysis of the glycomes and the modified glycomes. Furthermore, the invention is directed to stem cells carrying the modified glycomes on their surfaces. The glycomes are preferably analysed by profiling methods able to detect reproducibly and quantitatively numerous individual glycan structures at the same time. The most preferred type of the profile is a mass spectrometric profile. The invention specifically revealed novel target structures and is especially directed to the development of reagents recognizing the structures.

### FIELD OF THE INVENTION

The invention describes novel compositions of glycans, glycomes, from human multipotent stem cells, and especially novel subcompositions of the glycomes with specific monosaccharide compositions and glycan structures. The invention is further directed to methods for modifying the glycomes and analysis of the glycomes and the modified glycomes. Furthermore, the invention is directed to stem cells carrying the modified glycomes on their surfaces. The glycomes are preferably analysed by profiling methods able to detect reproducibly and quantitatively numerous individual glycan structures at the same time. The most preferred type of the profile is a mass spectrometric profile. The invention specifically revealed novel target structures and is especially directed to the development of reagents recognizing the structures.

### BACKGROUND OF THE INVENTION

Stem Cells

Stem cells are undifferentiated cells which can give rise to a succession of mature functional cells. For example, a hematopoietic stem cell may give rise to any of the different types of terminally differentiated blood cells. Embryonic stem (ES) cells are derived from the embryo and are pluripotent, thus possessing the capability of developing into any organ or tissue type or, at least potentially, into a complete embryo.

The first evidence for the existence of stem cells came from studies of embryonic carcinoma (EC) cells, the undifferentiated stem cells of teratocarcinomas, which are tumors derived from germ cells. These cells were found to be pluripotent and immortal, but possess limited developmental potential and abnormal karyotypes (Rossant and Papaioannou, Cell Differ 15,155-161, 1984). The glycans of cancer cells change by frequent mutations and the data from the cancer cell lines is not valid for ES cells. ES cells, on the other hand, are thought to retain greater developmental potential because they are derived from normal embryonic cells, without the selective pressures of the teratocarcinoma environment.

Pluripotent embryonic stem cells have traditionally been derived principally from two embryonic sources. One type can be isolated in culture from cells of the inner cell mass of a pre-implantation embryo and are termed embryonic stem (ES) cells (Evans and Kaufman, Nature 292,154-156, 1981; U.S. Pat. No. 6,200,806). A second type of pluripotent stem cell can be isolated from primordial germ cells (PGCS) in the mesenteric or genital ridges of embryos and has been termed embryonic germ cell (EG) (U.S. Pat. No. 5,453,357, U.S. Pat. No. 6,245,566). Both human ES and EG cells are pluripotent. This has been shown by differentiating cells in vitro and by injecting human cells into immunocompromised (SCUM) mice and analyzing resulting teratomas (U.S. Pat. No. 6,200,806). The term “stem cell” as used herein means stem cells including embryonic stem cells or embryonic type stem cells and stem cells differentiated thereof to more tissue specific stem cells.

The present invention provides novel markers and target structures and binders to these for especially embryonic stem cells. From hematopoietic CD34+ cells certain terminal structures such as terminal sialylated type two N-acetyllactosamines such as NeuNAcα3Galβ4GlcNAc (Magnani J. U.S. Pat. No. 6,362,010) has been suggested and there is indications for low expression of Slex type structures NeuNAcα3Galβ4(Fucα3)GlcNAc (Xia L et al Blood (2004) 104 (10) 3091-6). The invention is also directed to the NeuNAcα3Galβ4GlcNAc non-polylactosamine variants separately from specific characteristic O-glycans and N-glycans. Due to tissue specificity of glycosylation such data is not relevant to embryonic stem cells, which represent much earlier level of differentiation.

Human ES, EG and EC cells, as well as primate ES cells, express alkaline phosphatase, the stage-specific embryonic antigens SSEA-3 and SSEA-4, and surface proteoglycans that are recognized by the TRA-1-60; and TRA-1-81 antibodies. All these markers typically stain these cells, but are not entirely specific to stem cells, and thus cannot be used to isolate stem cells from organs or peripheral blood.

The SSEA-3 and SSEA-4 structures are known as galactosylgloboside and sialylgalactosylgloboside, which are among the few suggested structures on embryonic stem cells, though the nature of the structures in not ambitious. An antibody called K21 has been suggested to bind a sulfated polysaccharide on embryonic carcinoma cells (Badcock G et al Cancer Res (1999) 4715-19. Due to cell type, species, tissue and other specificity aspects of glycosylation (Furukawa, K., and Kobata, A. (1992) Curr. Opin. Struct. Biol. 3, 554-559, Gagneux, and Varki, A. (1999) Glycobiology 9, 747-755;Gawlitzek, M. et al. (1995), J. Biotechnol. 42, 117-131; Goelz, S., Kumar, R., Potvin, B., Sundaram, S., Brickelmaier, M., and Stanley, P. (1994) J. Biol. Chem. 269, 1033-1040; Kobata, A (1992) Eur. J. Biochem. 209 (2) 483-501.) This result does not indicate the presence of the structure on native embryonic stem cells. The present invention is directed to human stem cells.

Some low specificity plant lectin reagents have been reported in binding of embryonic stem cell like materials. Venable et al 2005, (Dev. Biol. 5:15) measured lectins the binding of SSEA-4 antibody positive subpopulation of embryonic stem cells. This approach suffers obvious problems. It does not tell the expression of the structures in native non-selected embryonic stem cells. The SSEA-4 was chosen select especially pluripotent stem cells. The scientists of the same Bresagen company have further revealed that actual role of SSEA-4 with the specific stem cell lines is not relevant for the pluripotency.

The work does not reveal: 1) The actual amount of molecules binding to the lectins or 2) presence of any molecules due to defects caused by the cell sorting and experimental problems such as trypsination of the cells. It is really alerting that the cells were trypsinized, which removes protein and then enriched by possible glycolipid binding SSEA4 antibody and secondary antimouse antibody, fixed with paraformaldehyde without removing the antibodies, and labelled by simultaneous with lectin and the same antibody and then the observed glycan profile is the similar as revealed by lectin analysis by same scientist for antibody glycosylation (M. Pierce US2005) or 3) the actual structures, which are bound by the lectins. To reveal the possible residual binding to the cells would require analysis of of the glycosylations of the antibodies used (sources and lots not revealed).

The purity of the SSEA-4 positive cells was reported to be 98-99 %, which is unusually high. The quantitation of the binding is not clear as FIG. 18 shows about 10 % binding by lectins LTL and DBA, which are not bound to hESC-cells 3^{rd} page, column 2, paragraph 2 and by immunocytochemistry 4 the page last line.

It appears that skilled artisan would consider the results of Venable et al such convenient colocalization of SSEA-4 and the lectin binding by binding of the lectins to the anti-SSEA-4 antibody. It appears that the more rare binding would reflect lower proportion of the terminal epitope per antibody molecule leading to lower density of the labellable antibodies. It is also realized that the non-controlled cell culture process with animal derived material would lead to contamination of the cells by N-glycolyl-neuraminic acid, which may be recognized by anti-mouse antibodies used as secondary antibody (not defined what kind of anti-mouse) used in purification and analysis of purity, which could lead to conveniently high cell purity.

The work is directed only to the “pluripotent” embryonic stem cells associated with SSEA-4 labelling and not to differentiated variants thereof as the present invention. The results indicated possible binding (likely on the antibodies) to certain potential monosaccharide epitopes (6^{th} page, Table 21, and column 2) such Gal and Galactosamine for RCA (ricin, inhitable by Gal or lactose), GlcNAc for TL (tomato lectin), Man or Glc for ConA, Sialic acid/Sialic acid α6GalNAc for SNA, Manα for HHL; lectins with partial binding not correlating with SSEA-4: GalNAc/GalNAcβ4Gal (in text) WFA, Gal for PNA, and Sialic acid/Sialic acid α6GalNAc for SNA; and lectins associated by part of SSEA-4 cells were indicated to bind Gal by PHA-L and PHA-E, GalNAc by VVA and Fuc by UEA, and Gal by MAA (inhibited by lactose). UEA binding was discussed with reference as endothelial marker and O-linked fucose which is directly bound to Ser (Thr) on protein. The background has indicated a H type 2 specificity for the endothelial UEA receptor. The specificities of the lectins are somewhat unusual, but the product codes or isolectin numbers/names of the lectins were not indicated (except for PHA-E and PHA-L) and it is known that plants contain numerous isolectins with varying specificities.

Wearne K A et al Glycobiology (2006) 16 (10) 981-990 studied also staining of embryonic stem cells by plant lectins. The data using the low specificity reagents does not reveal exact glycan structures and specifically not the elongated structure on specific glycan core structures as described by the present invention for human embryonic stem cells nor useful antibody reagent specificities for specific recognition of terminal epitopes. The authors guess some binding/non-binding structures based on the lectin bindings, which appear to be at least partially different from ones revealed by the invention indicating possible technical problems. This work does not imply any other type of usefulness of the lectins in other cell/cell materials directed methods. The Wearne data describes embryonic bodies, which is stage 2 differentiation in present work, but appears to lack data about further differentiated cells such as stage 3 cells.

The present invention revealed specific structures by mass spectrometric profiling, NMR spectrometry and binding reagents including glycan modifying enzymes. The lectins are in general low specificity molecules. The present invention revealed binding epitopes larger than the previously described monosaccharide epitopes. The larger epitopes allowed us to design more specific binding substances with typical binding specificities of at least disaccharides. The invention also revealed lectin reagents with specified with useful specificities for analysis of native embryonic stem cells without selection against an uncontrolled marker and/or coating with an antibody or two from different species. Clearly the binding to native embryonic stem cells is different as the binding with MAA was clear to most of cells, there was differences between cell line so that RCA, LTA and UEA was clearly binding a HESC cell line but not another.

Methods for separation and use of stem cells are known in the art.

There have been great efforts toward isolating pluripotent or multipotent stem cells, in earlier differentiation stages than hematopoietic stem cells, in substantially pure or pure form for diagnosis, replacement treatment and gene therapy purposes. Stem cells are important targets for gene therapy, where the inserted genes are intended to promote the health of the individual into whom the stem cells are transplanted. In addition, the ability to isolate stem cells may serve in the treatment of lymphomas and leukemias, as well as other neoplastic conditions where the stem cells are purified from tumor cells in the bone marrow or peripheral blood, and reinfused into a patient after myelosuppressive or myeloablative chemotherapy.

Multiple adult stem cell populations have been discovered from various adult tissues. In addition to hematopoietic stem cells, neural stem cells were identified in adult mammalian central nervous system (Ourednik et al. Clin. Genet. 56, 267, 1999). Adult stem cells have also been identified from epithelial and adipose tissues (Zuk et al. Tissue Engineering 7, 211, 2001). Recent studies have demonstrated that certain somatic stem cells appear to have the ability to differentiate into cells of a completely different lineage (Pfendler K C and Kawase E, Obstet Gynecol Surv 58, 197-208, 2003). Monocyte derived (Zhao et al. Proc. Natl. Acad. Sci. USA 100, 2426-2431, 2003) and mesodermal derived (Schwartz et al. J. Clin. Invest 109, 1291-1301, 2002) cells that possess some multipotent characteristics were identified. The presence of multipotent “embryonic-like” progenitor cells in blood was suggested also by in-vivo experiments following bone marrow transplantations (Zhao et al. Brain Res Protoc 11, 38-45, 2003). However, such multipotent “embryonic-like” stem cells cannot be identified and isolated using the known markers.

The present invention provides methods of identifying, characterizing and separating stem cells having characteristics of embryonic stem (ES) cells for diagnostic, therapy and tissue engineering. In particular, the present invention provides methods of identifying, selecting and separating embryonic stem cells or fetal cells from maternal blood and to reagents for use in prenatal diagnosis and tissue engineering methods. The present invention provides for the first time a specific marker/binder/binding agent that can be used for identification, separation and characterization of valuable stem cells from tissues and organs, overcoming the ethical and logistical difficulties in the currently available methods for obtaining embryonic stem cells.

The present invention overcomes the limitations of known binders/markers for identification and separation of embryonic or fetal stem cells by disclosing a very specific type of marker/binder, which does not react with differentiated somatic maternal cell types. In other aspect of the invention, a specific binder/marker/binding agent is provided which does not react, i.e. is not expressed on feeder cells, thus enabling positive selection of feeder cells and negative selection of stem cells.

By way of exemplification, the binder to Formulas according to the invention are now disclosed as useful for identifying, selecting and isolating pluripotent or multipotent stem cells including embryonic and embryonic type stem cells, which have the capability of differentiating into varied cell lineages.

According to one aspect of the present invention a novel method for identifying pluripotent or multipotent stem cells in peripheral blood and other organs is disclosed. According to this aspect an embryonic stem cell binder/marker is selected based on its selective expression in stem cells and/or germ stem cells and its absence in differentiated somatic cells and/or feeder cells. Thus, glycan structures expressed in stem cells are used according to the present invention as selective binders/markers for isolation of pluripotent or multipotent stem cells from blood, tissue and organs. Preferably the blood cells and tissue samples are of mammalian origin, more preferably human origin.

According to a specific embodiment the present invention provides a method for identifying a selective embryonic stem cell binder/marker comprising the steps of:

A method for identifying a selective stem cell binder to a glycan structure of Formula (I) which comprises:

i. selecting a glycan structure exhibiting specific expression in/on stem cells and absence of expression in/on feeder cells and/or differentiated somatic cells; ii. and confirming the binding of binder to the glycan structure in/on stem cells.

By way of a non-limiting example, embryonic type, stem cells selected using the binder may be used in regenerating the hematopoietic or other tissue system of a host deficient in any class of stem cells. A host that is diseased can be treated by removal of bone marrow, isolation of stem cells and treatment with drugs or irradiation prior to re-engraftment of stem cells. The novel markers of the present invention may be used for identifying and isolating various embryonic type stem cells; detecting and evaluating growth factors relevant to stem cell self-regeneration; the development of stem cell lineages; and assaying for factors associated with stem cell development.

### BRIEF DESCRIPTION OF THE DRAWINGS

FIG. 1. Portrait of the hESC N-glycome. A. Mass spectrometric profiling of the most abundant 50 neutral N-glycans (A) and 50 sialylated N-glycans (B) of the four hESC lines (blue columns/left), four EB samples (middle columns), and four stage 3 differentiated cell samples (light columns/right). The columns indicate the mean abundance of each glycan signal (% of the total glycan signals). Proposed N-glycan monosaccharide compositions are indicated on the x-axis: S: NeuAc, H: Hex, N: HexNAc, F: dHex, Ac: acetyl. The mass spectrometric glycan profile was rearranged and the glycan signals grouped in the main N-glycan structure classes. Glycan signals in the group ‘Other’ are marked with m/z ratio of their [M+Na]+ (left panel) or [M−H]− ions (right panel). The isolated N-glycan fractions of hESC were structurally analyzed by proton NMR spectroscopy to characterize the major N-glycan core and backbone structures, and specific exoglycosidase digestions with α-mannosidase (Jack beans), α1,2- and α1,3/4-fucosidases (X. manihotis/recombinant), β1,4-galactosidase (S. pneumoniae), and neuraminidase (A. ureafaciens) to characterize the non-reducing terminal epitopes. Structures proposed for the major N-glycan signals are indicated by schematic drawings in the bar diagram. The major sialylated N-glycan structures are based on the trimannosyl core with or without core fucosylation as demonstrated in the NMR analysis. Galactose linkages or branch specificity of the antennae are not specified in the present data. The Lewis x antigen was detected in the same cells by monoclonal antibody staining (not shown).

FIG. 2. Mass spectrometric profiling of human embryonic stem cell and differentiated cell N-glycans. A. Neutral N-glycans and B. 50 most abundant sialylated N-glycans of the four hESC lines (blue columns), embryoid bodies derived from FES 29 and FES 30 hESC lines (EB, red columns), and stage 3 differentiated cells derived from FES 29 (st.3, white columns). The columns indicate the mean abundance of each glycan signal (% of the total detected glycan signals). Error bars indicate the range of detected signal intensities. Proposed monosaccharide compositions are indicated on the x-axis. H: hexose, N: N-acetylhexosamine, F: deoxyhexose, S: N-acetylneuraminic acid, G: N-glycolylneuraminic acid.

FIG. 3. A. Classification rules for human N-glycan biosynthetic groups. The minimal structures of each biosynthetic group (solid lines) form the basis for the classification rules. Variation of the basic structures by additional monosaccharide units (dashed lines) generates complexity to stem cell glycosylation as revealed in the present study. H: hexose, N: N-acetylhexosamine, F: deoxyhexose, S: N-acetylneuraminic acid. B. Diagram showing relative differences in N-glycan classes between hESC and stage 3 differentiated cells (st.3). Although the major N-glycan classes are expressed in both hESC and the differentiated cell types, their relative proportions are changed during hESC differentiation. Complex fucosylation (F≧2) of sialylated N-glycans as well as high-mannose type and complex-type N-glycans were identified as the major hESC-associated N-glycosylation features. In contrast, fucosylation as such (F≧1) was not similarly specific. Hybrid-type or monoantennary, low-mannose type, and terminal N-acetylhexosamine (N>H≧2 or N=H≧5) type N-glycans were associated with differentiated cells. The relative differences were calculated according to Equation 2 from the N-glycan profiles (Supplementary Table S5). Schematic examples of glycan structures included in each glycan class are inserted in the diagram. Glycan symbols: ▪, N-acetyl-D-glucosamine; ◯, D-mannose; , D-galactose; ♦, N-acetylneuraminic acid; Δ, L-fucose; □, N-acetyl-D-galactosamine.

FIG. 4. The major N-glycan structures in hESC N-glycome were determined by MALDI-TOF mass spectrometry combined with exoglycosidase digestion and proton NMR spectroscopy. A, High-mannose type N-glycans with five to nine mannose residues dominated the neutral N-glycan fraction. B, In the sialylated N-glycan fraction, the most abundant components were biantennary complex-type N-glycans with either α2,3 or α2,6-sialylated type II N-acetyllactosamine antennae and with or without core α1,6-fucosylation. Glycan symbols: see legend of FIG. 3; lines indicate glycosidic linkages between monosaccharide residues; dashed lines indicate the presence of multiple structures;→Asn indicates site of linkage to glycoprotein.

FIG. 5. Statistical discrimination analysis of the four hESC lines, embryoid bodies derived from FES 29 and FES 30 hESC lines (EB), and stage 3 differentiated cells derived from FES 29 (st.3). The calculation of the glycan score is detailed in the Supplementary data.

FIG. 6. Lectin staining of hESC colonies grown on mouse feeder cell layers, with A, Maackia amurensis agglutinin (MAA) that recognizes α2,3-sialylated glycans, and with B, Pisum sativum agglutinin (PSA) that recognizes N-glycan core residues. PSA recognized hESC only after cell permeabilization (data not shown). Mouse fibroblasts had complementary staining patterns with both lectins, indicating that their surface glycans are clearly different from hESC. C, The results indicate that mannosylated N-glycans are localized primarily in the intracellular compartments in hESC, whereas α2,3-sialylated glycans occur on the cell surface.

FIG. 7. 50 most abundant signals from the neutral N-glycome of human embryonic stem cells.

FIG. 8. Hybrid and complex N-glycans picked from the 50 most abundant signals from the neutral N-glycome of human embryonic stem cells.

FIG. 9. 50 most abundant signals from the acidic N-glycome of human embryonic stem cells.

FIG. 10. (A) Hybrid N-glycans of human embryonic stem cells and changes in their relative abundance during differentiation. (B) Enlargement of the X-axis of (A).

FIG. 11. High mannose N-glycans (Man≧5) of human embryonic stem cells and changes in their relative abundance during differentiation.

FIG. 12. “Low mannose” N-glycans (Man 1-4) of human embryonic stem cells and changes in their relative abundance during differentiation.

FIG. 13. (A) Fucosylated N-glycans of human embryonic stem cells and changes in their relative abundance during differentiation. (B) Enlargement of the X-axis of (A).

FIG. 14. (A) “Complexly fucosylated” (Fuc≧2) N-glycans of human embryonic stem cells and changes in their relative abundance during differentiation. (B) Enlargement of the X-axis of (A).

FIG. 15. Sulfated N-glycans of human embryonic stem cells and changes in their relative abundance during differentiation.

FIG. 16. Large N-glycans (H≧7, N≧6) of human embryonic stem cells and changes in their relative abundance during differentiation.

FIG. 17. Portrait of the hESC N-glycome. MALDI-TOF mass spectrometric profiling of the most abundant 50 neutral N-glycans (A.) and 50 sialylated N-glycans (B.) of the four hESC lines FES 21, 22, 29, and 30 (black columns), four EB samples (gray columns), and four st.3 differentiated cell samples (white columns) derived from the four hESC lines, respectively. The columns indicate the mean abundance of each glycan signal (% of the total glycan signals). The observed m/z values for either [M+Na]+ or [M−H]− ions for the neutral and sialylated N-glycan fractions, respectively, are indicated on the x-axis. Proposed monosaccharide compositions and N-glycan types are presented in Table 21.

FIG. 18. Detection of hESC glycans by structure-specific reagents. To study the localization of the detected glycan components in hESC, stem cell colonies grown on mouse feeder cell layers were labeled by fluoresceinated glycan-specific reagents selected based on the analysis results. A. The HESC surfaces were stained by Maackia amurensis agglutinin (MAA), indicating that α2,3-sialylated glycans are abundant on hESC but not on feeder cells (MEF, mouse feeder cells). B. In contrast, the hESC cell surfaces were not stained by Pisum sativum agglutinin (PSA) that recognized mouse feeder cells, indicating that α-mannosylated glycans are not abundant on hESC surfaces but are present on mouse feeder cells. C. Addition of 3′-sialyllactose blocks MAA binding, and D. addition of D-mannose blocks PSA binding.

FIG. 19. hESC-associated glycan signals selected from the 50 most abundant sialylated N-glycan signals of the analyzed hESC, EB, and st.3 samples (data taken from FIG. 1.B).

FIG. 20. Differentiated cell associated glycan signals selected from the 50 most abundant sialylated N-glycan signals of the analyzed hESC, EB, and st.3 samples (data taken from FIG. 17.B).

FIG. 21. A) Baboon polyclonal anti-Galα3Gal antibody staining of mouse fibroblast feeder cells (left) showing absence of staining in hESC colony (right). B) UEA (Ulex Europaeus) lectin staining of stage 3 human embryonic stem cells. FES 30 line.

FIG. 22. A) UEA lectin staining of FES22 human embryonic stem cells (pluripotent, undifferentiated). B) UEA staining of FES30 human embryonic stem cells (pluripotent, undifferentiated).

FIG. 23. A) RCA lectin staining of FES22 human embryonic stem cells (pluripotent, undifferentiated). B) WFA lectin staining of FES30 human embryonic stem cells (pluripotent, undifferentiated).

FIG. 24. A) PWA lectin staining of FES30 human embryonic stem cells (pluripotent, undifferentiated). B) PNA lectin staining of FES30 human embryonic stem cells (pluripotent, undifferentiated).

FIG. 25. A) GF 284 immunostaining of FES30 human embryonic stem cell line. Immunostaining is seen in the edges of colonies in cells of early differentiation (10× magnification). Mouse feeder cells do not stain. B) Detail of GF284 as seen in 40× magnification. This antibody is suitable for detecting a subset of hESC lineage.

FIG. 26. A) GF 287 immunostaining of FES30 human embryonic stem cell line. Immunostaining is seen throughout the colonies (10× magnification). Mouse feeder cells do not stain. B) Detail of GF287 as seen in 40× magnification. This antibody is suitable for detecting undifferentiated, pluripotent stem cells.

FIG. 27. A) GF 288 immunostaining of FES30 human embryonic stem cells. Immunostaining is seen mostly in the edges of colonies in cells of early differentiation (10× magnification). Mouse feeder cells do not stain. B) Detail of GF288 as seen in 40× magnification. This antibody is suitable for detecting a subset of hESC lineage

FIG. 28. The canonical means of the first discriminant analysis for neutral hESC, EB and st3. Root 1 is represented on the x-axis and Root 2 on the y-axis. From the figure we can see that the means are further differentiated on the x-axis and therefore we use Root 1 to determine the function.

FIG. 29. The canonical means of the second minimal discriminant analysis for neutral glycans from hESC, EB and st3 (5 masses). Root 1 is represented on the x-axis and Root 2 on the y-axis.

FIG. 30. The canonical means of the first minimal discriminant analysis for neutral glycans from hESC, EB and st3 (4 masses). Root 1 is represented on the x-axis and Root 2 on the y-axis.

FIG. 31. Lectin FACS of hESCs. hESCs were detached with EDTA, washed with FCS-PBS. FES30 cells were double staining with SSEA-3+.

FIG. 32. FACS analysis using various antibodies. The cells were detached with EDTA and washed with buffer containing FCS.

### DESCRIPTION OF THE INVENTION

Related data and specification was presented in PCT FI 2006/050336, for US proceedings and when relevant for other countries the applications are included as reference.

The present invention revealed novel stem cell specific glycans, with specific monosaccharide compositions and associated with differentiation status of stem cells and/or several types of stem cells and/or the differentiation levels of one stem cell type and/or lineage specific differences between stem cell lines.

The present invention is directed to human embryonic type stem cells and stem cells and tissue precursors differentiated thereof. It is realized that ethical considerations may restrict patenting of actual embryonic stem cells derived from human embryos, but there is numerous technologies to produce equivalent materials with less or no ethical concerns involved. Furthermore non destructive analysis of stem cells should not involve ethical problems.

Preferred Target Cell Populations and Types for Analysis According to the Invention

Human Embryonic Type Stem Cells

Under broadest embodiment the present invention is directed to all types of human embryonic type stem cells, meaning fresh and cultured human embryonic type stem cells.

The stem cells according to the invention do not include traditional cancer cell lines, which may differentiate to resemble natural cells, but represent non-natural development, which is typically due to chromosomal alteration or viral transfection. It is realized that the data from embryonal carcinomas (EC) and EC cell lines is not relevant for embryonic stem cells.

The embryonic stem cells include all types of non-malignant embryonic multipotent or totipotent cells capable of differentiating to other cell types. The embryonic stem cells have special capacity stay as stem cells after cell division, the self-renewal capacity. The preferred differentiated derivatives of embryonic stem cells includes embryonic bodies, also referred as stage 2 differentiated embryonic stem cells and stage three differentiated embryonic stem cells. In a preferred embodiment the the stage 3 embryonic stem cells have at least partial characteristics of specific tissue or more preferably characteristics of a specific tissue stem cells.

Under the broadest embodiment for the human stem cells, the present invention describes novel special glycan profiles and novel analytics, reagents and other methods directed to the glycan profiles. The invention shows special differences in cell populations with regard to the novel glycan profiles of human stem cells.

The present invention is further directed to the novel structures and related inventions with regard to the preferred cell populations according to the invention. The present invention is further directed to specific glycan structures, especially terminal epitopes, with regard to specific preferred cell population for which the structures are new.

Embryonic Type Cell Populations

The present invention is specifically directed to methods directed to embryonic type or “embryonic like” cell populations, preferably when the use does not involve commercial or industrial use of human embryos and/or involve destruction of human embryos. The invention is under a specific embodiment directed to use of embryonic cells and embryo derived materials such as embryonic stem cells, whenever or wherever it is legally acceptable. It is realized that the legislation varies between countries and regions. The inventors reserve possibility to disclaim legally restricted types of embryonic stem cells.

The present invention is further directed to use of embryonic-related, discarded or spontaneously damaged material, which would not be viable as human embryo and cannot be considered as a human embryo. In yet another embodiment the present invention is directed to use of accidentally damaged embryonic material, which would not be viable as human embryo and cannot be considered as human embryo. Gene technology and embryonic biopsy based methods producing ES cells from embryos without damaging the embryo to produce embryonic or embryonic type stem cells are expected to produce ethically acceptable or more cells.

In a preferred embodiment the invention is directed to embryonic type stem cells, which are produced from other cell types by programming the cells to undifferentiated status corresponding to embryonic stem cells or cells corresponding to the preferred differentiated variants of the ES cells.

The invention is further directed to cell materials equivalent to the cell materials according to the invention. It is further realized that functionally and even biologically similar cells may be obtained by artificial methods including cloning technologies.

N-Glycan Structures and Compositions Associated with Differentiation of Stem Cells

The invention revealed specific glycan monosaccharide compositions and corresponding structures, which associated with
i) non-differentiated human embryonic stem cells, hESCs (stage 1) or
ii) stage 2 (embryoid bodies) and/or
iii) stage 3 differentiated cells differentiated from the hESCs.

It is realized that the structures revealed are useful for the characterization of the cells at different stages of development. The invention is directed to the use of the structures as markers for differentiation of embryonic stem cells. The invention is further directed to the use of the specific glycans as markers enriched or increased at specific level of differentiation for the analysis of the cells at specific differentiation level.

Glycan Structures and Compositions are Associated with Individual Specific Differences between Stem Cell Lines or Batches.

The invention further revealed that specific glycan types are presented in the embryonic stem cell preparations on a specific differentiation stage in varying manner. It is realized that such individually varying glycans are useful for characterization of individual stem cell lines and batches. The specific structures of a individual cell preparation are useful for comparison and standardization of stem cell lines and cells prepared thereof.

The specific structures of a individual cell preparation are used for characterization of usefulness of specific stem cell line or batch or preparation for stem cell therapy in a patient, who may have antibodies or cell mediated immune defense recognizing the individually varying glycans.

The invention is especially directed to analysis of glycans with large and moderate variations as described in example 3.

Recognition of Multiple Structures

The invention revealed multiple glycan structures and corresponding mass spectrometric signals, which are characteristic for the stem cell populations according to the invention. In a preferred embodiment the invention is directed to recognition of specific combinations glycans such as whole glycans and/or corresponding signals, such as mass spectrometric signals and/or specific structural epitopes, preferably non-reducing end terminal glycans structures.

It is realized that certain combination of structures are useful for detection because the change of structures can be correlated with the status of the cell, in a preferred embodiment the differentiation status of the cells is correlated with the glycans. The invention specifically revealed glycans changing during the differentiation of the cells. It was revealed that certain glycan structures are increased and others decreased during differentiation of cells. The invention is directed to use of combinations of structures changing similarly during differentiation and/or structures changing differently (at least one decreasing and at least one decreasing).

Analysis Methods by Mass Spectrometry or Specific Binding Reagents

The invention is specifically directed to the recognition of the terminal structures by either specific binder reagents and/or by mass spectrometric profiling of the glycan structures.

In a preferred embodiment the invention is directed to the recognition of the structures and/or compositions based on mass spectrometric signals corresponding to the structures.

The preferred binder reagents are directed to characteristic epitopes of the structures such as terminal epitopes and/or characteristic branching epitopes, such as monoantennary structures comprising a Manα-branch or not comprising a Manα-branch.

The preferred binder is an antibody, more preferably a monoclonal antibody.

In a preferred embodiment the invention is directed to a monoclonal antibody specifically recognizing at least one of the terminal epitope structures according to the invention.

Recognition of Preferred Terminal Epitopes

The invention is in a preferred embodiment directed to the analysis of the stem cells by specific antibodies and other binding reagents recognizing preferred structural epitopes according to the invention.

The preferred structural epitopes includes non-reducing end terminal Gal/GalNAcβ3/4-epitope comprising structures and sialylated and/or fucosylated derivatives thereof. The invention is directed to recognition of at at least one N-acetylactos

Non-Reducing End Terminal Gal(NAc)Beta Structures

Terminal Galactose epitopes including
i) terminal N-acetyllactosamines Galβ3GlcNAc and/or Galβ4GlcNAc, and fucosylated branched variants thereof such as Lewis a [Galβ3(Fucα4)GlcNAc] and Lewis x [Galβ4(Fucα3)GlcNAc]
ii) O-glycan core structures including Galβ3GalNAcα in linear core I epitope and/or branched Galβ3(R-GlcNAcβ6)GalNAcα,

iii) Glycolipid structures with terminal Galβ3GalNAcβ-structures

Terminal GalNAc epitopes including
i) terminal di-N-acetyllactosediamine GalNAcβ4GlcNAc (LacdiNAc), and α3fucosylated derivative thereof, LexNAc [GalNAcβ4(Fucα3)GlcNAc]
ii) Glycolipid structures with terminal GalNAcβ3Gal-structures

Sialylated Non-Reducing End Terminal Gal(NAc)Beta Structures

The preferred terminal sialylated Gal(NAc) epitopes including,

The preferred sialic acid is (SA) such Neu5Ac or Neu5Gc.
i) terminal sialyl-N-acetyllactosamines SAα3/6Galβ3GlcNAc and/or SAα3/6Galβ4GlcNAc, and fucosylated branched variants thereof such as sialyl-Lewis a [SAα3Galβ3(Fucα4)GlcNAc] and sialyl-Lewis x [SAα3Galβ4(Fucα3)GlcNAc]
ii) sialylated O-glycan core structures including SAα3Galβ3GalNAcα in linear core I epitope or disialyl-structures SAα3Galβ3(SAα6)GalNAcα, and/or branched SAα3Galβ3(R-GlcNAcβ6)GalNAcα,
iii) Glycolipid structures with terminal SAα3Galβ3GalNAcβ-structures and disialostructures SAα3Galβ3 (SAα6)GalNAcβ, disialosyl-Tn).

Terminal sialylated GalNAc epitopes including sialylated GalNAcβ3/4-structures
i) terminal sialyl di-N-acetyllactosediamine SAαGalNAcβ4GlcNAc, more preferably SAα6GalNAcβ4GlcNAc

Fucosylated Non-Reducing End Terminal Galbeta Structures

The position 2 of galactose carrying N-acetyl group in GalNAc can be fucosylated to a preferred structure group with similarity to the terminal GalNAc structures The preferred terminal fucosylated Gal epitopes includes,
i) terminal fucosyl-N-acetyllactosamines Fucα2Galβ3GlcNAc and/or Fucα2Galβ4GlcNAc, and fucosylated branched variants thereof such as Lewis b [Fucα2Galβ3(Fucα4)GlcNAc] and Lewis y [Fucα2Galβ4(Fucα3)GlcNAc]
ii) fucosylated O-glycan core structures including Fucα2Galβ3GalNAcα in linear core I epitope and/or branched Fucα2Galβ3(R-GlcNAcβ6)GalNAcα,
iii) Glycolipid structures with terminal Fucα2Galβ3GalNAcβ-structures.

Terminal Structural Epitopes

We have previously revealed glycome compositions of human glycomes, here we provide structural terminal epitopes useful for the characterization of stem cell glycomes, especially by specific binders.

The examples of characteristic altering terminal structures includes expression of competing terminal epitopes created as modification of key homologous core Galβ-epitopes, with either the same monosaccharides with difference in linkage position Galβ3GlcNAc, and analogue with either the same monosaccharides with difference in linkage position Galβ4GlcNAc; or the with the same linkage but 4-position epimeric backbone Galβ3GalNAc. These can be presented by specific core structures modifying the biological recognition and function of the structures. Another common feature is that the similar Galβ-structures are expressed both as protein linked (O— and N-glycan) and lipid linked (glycolipid structures). As an alternative for α2-fucosylation the terminal Gal may comprise NAc group on the same 2 position as the fucose. This leads to homologous epitopes GalNAcβ4GlcNAc and yet related GalNAcβ3Gal-structure on characteristic special glycolipid according to the invention.

The invention is directed to novel terminal disaccharide and derivative epitopes from human stem cells, preferably from human embryonic type stem cells. It should realized that glycosylations are species, cell and tissue specific and results from cancer cells usually differ dramatically from normal cells, thus the vast and varying glycosylation data obtained from human embryonal carcinomas are not actually relevant or obvious to human embryonic stem cells (unless accidentally appeared similar). Additionally the exact differentiation level of teratocarcinomas cannot be known, so comparison of terminal epitope under specific modification machinery cannot be known. The terminal structures by specific binding molecules including glycosidases and antibodies and chemical analysis of the structures.

The present invention reveals group of terminal Gal(NAc)β1-3/4Hex(NAc) structures, which carry similar modifications by specific fucosylation/NAc-modification, and sialylation on corresponding positions of the terminal disaccharide epitopes. It is realized that the terminal structures are regulated by genetically controlled homologous family of fucosyltransferases and sialyltransferases. The regulation creates a characteristic structural patterns for communication between cells and recognition by other specific binder to be used for analysis of the cells. The key epitopes are presented in the TABLE 21. The data reveals characteristic patterns of the terminal epitopes for each types of cells, such as for example expression on hESC-cells generally much Fucα-structures such as Fucα2-structures on type 1 lactosamine (Galβ3GlcNAc), similarly β3-linked core I Galβ3GlcNAcα, and type 4 structure which is present on specific type of glycolipids and expression of α3-fucosylated structures, while α6-sialic on type II N-acetyllactosamine appear on N-glycans of embryoid bodies and st3 embryonic stem cells. E.g. terminal type lactosamine and poly-lactosamines differentiate stem cells with different status such as differentiation status. The terminal Galβ-information is preferably combined with information about information about other preferred terminal structures such as sialylated and/or fucosylated structures.

The invention is directed especially to high specificity binding molecules such as monoclonal antibodies for the recognition of the structures.

The structures can be presented by Formula T1. the formula describes first monosaccharide residue on left, which is a β-D-galactopyranosyl structure linked to either 3 or 4-position of the α- or β-D-(2-deoxy-2-acetamido)galactopyranosyl structure, when R₅ is OH, or β-D-(2-deoxy-2-acetamido)glucopyranosyl, when R₄ comprises O—. The unspecified stereochemistry of the reducing end in formulas T1 and T2 is indicated additionally (in claims) with curved line. The sialic acid residues can be linked to 3 or 6-position of Gal or 6-position of GlcNAc and fucose residues to position 2 of Gal or 3- or 4-position of GlcNAc or position 3 of Glc. The invention is directed to Galactosyl-globoside type structures comprising terminal Fucα2-revealed as novel terminal epitope Fucα2Galβ3GalNAcβ or Galβ3GalNAcβGalα3-comprising isoglobotructures revealed from the embryonic type cells.

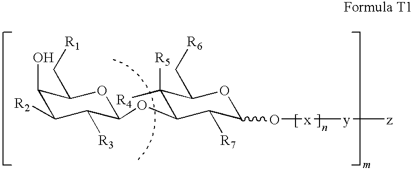

wherein

X is linkage position

R₁, R₂, and R₆ are OH or glycosidically linked monosaccharide residue Sialic acid, preferably Neu5Acα2 or Neu5Gc α2, most preferably Neu5Acα2 or

R₃, is OH or glycosidically linked monosaccharide residue Fucα1 (L-fucose) or N-acetyl (N-acetamido, NCOCH₃);

R₄, is H, OH or glycosidically linked monosaccharide residue Fucα1 (L-fucose),

R₅ is OH, when R₄ is H, and R₅ is H, when R₄ is not H;

R7 is N-acetyl or OH

X is natural oligosaccharide backbone structure from the cells, preferably N-glycan, O-glycan or glycolipid structure; or X is nothing, when n is 0,

Y is linker group preferably oxygen for O-glycans and O-linked terminal oligosaccharides and glycolipids and N for N-glycans or nothing when n is 0;

Z is the carrier structure, preferably natural carrier produced by the cells, such as protein or lipid, which is preferably a ceramide or branched glycan core structure on the carrier or H;

The arch indicates that the linkage from the galactopyranosyl is either to position 3 or to position 4 of the residue on the left and that the R4 structure is in the other position 4 or 3;

n is an integer 0 or 1, and m is an integer from 1 to 1000, preferably 1 to 100, and most preferably 1 to 10 (the number of the glycans on the carrier),

With the provisions that one of R2 and R3 is OH or R3 is N-acetyl,

R6 is OH, when the first residue on left is linked to position 4 of the residue on right:

X is not Galα4Galβ4Glc, (the core structure of SSEA-3 or 4) or R3 is Fucosyl

R7 is preferably N-acetyl, when the first residue on left is linked to position 3 of the residue on right:

Preferred terminal β3-linked subgroup is represented by Formula T2 indicating the situation, when the first residue on the left is linked to the 3 position with backbone structures Gal(NAc)β3Gal/GlcNAc.

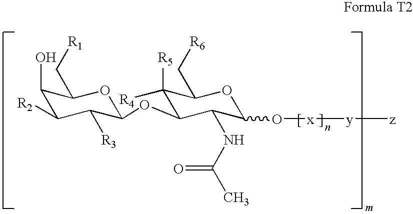

Wherein the variables including R₁ to R₇ are as described for T1

Preferred terminal β4-linked subgroup is represented by the Formula 3

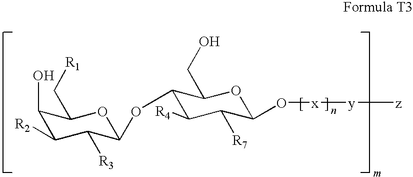

Wherein the variables including R₁ to R₄ and R7 are as described for T1 with the provision that R₄, is OH or glycosidically linked monosaccharide residue Fucα1 (L-fucose),

Alternatively the epitope of the terminal structure can be represented by Formulas T4 and T5

Core Galβ-epitopes formula T4:

Galβ1-xHex(NAc)ₚ,

x is linkage position 3 or 4,

and Hex is Gal or Glc

with provision

p is 0 or 1

when x is linkage position 3, p is 1 and HexNAc is GlcNAc or GalNAc,

and when x is linkage position 4, Hex is Glc.

The core Galβ1-3/4 epitope is optionally substituted to hydroxyl by one or two structures SAαor Fucα, preferably selected from the group

Gal linked SAα3 or SAα6 or Fucα2, and

Glc linked Fucα3 or GlcNAc linked Fucα3/4.

[Mα]ₘGalβ1-x[Nα]ₙHex(NAc)ₚ, Formula T5

wherein m, n and p are integers 0, or 1, independently

Hex is Gal or Glc,

X is linkage position

M and N are monosaccharide residues being independently nothing (free hydroxyl groups at the positions) and/or

SA which is Sialic acid linked to 3-position of Gal or/and 6-position of HexNAc and/or

Fuc (L-fucose) residue linked to 2-position of Gal

and/or 3 or 4 position of HexNAc, when Gal is linked to the other position (4 or 3),

and HexNAc is GlcNAc, or 3-position of Glc when Gal is linked to the other position (3),

with the provision that sum of m and n is 2

preferably m and n are 0 or 1, independently.

The exact structural details are essential for optimal recognition by specific binding molecules designed for the analysis and/or manipulation of the cells.

The terminal key Galβ-epitopes are modified by the same modification monosaccharides NeuX (X is 5 position modification Ac or Gc of sialic acid) or Fuc, with the same linkage type alfa (modifying the same hydroxyl-positions in both structures.

NeuXα3, Fucα2 on the terminal Galβ of all the epitopes and

NeuXα6 modifying the terminal Galβ of Galβ4GlcNAc, or HexNAc, when linkage is 6 competing or Fucα modifying the free axial primary hydroxyl left in GlcNAc (there is no free axial hydroxyl in GalNAc-residue).

The preferred structures can be divided to preferred Galβ1-3 structures analogously to T2,

[Mα]ₘGalβ1-3[Nα]ₙHexNAc, Formula T6:

Wherein the variables are as described for T5.

The preferred structures can be divided to preferred Galβ1-4 structures analogously to T4,

[Mα]ₘGalβ1-4[Nα]ₙGlc(NAc)ₚ, Formula T7:

Wherein the variables are as described for T5.

These are preferred type II N-acetyllactosamine structures and related lactosylderivatives, in a preferred embodiment p is 1 and the structures includes only type 2 N-acetyllactosamines. The invention revealed that the these are very useful for recognition of specific subtypes of embryonic type stem cells or differentiated variants thereof (tissue type specifically differentiated embryonic stem cells or various stages of embryonic stem cells). It is notable that various fucosyl- and or sialic acid modification created characteristic pattern for the stem cell type.

Preferred Type I and Type II N-Acetyllactosamine Structures

The preferred structures can be divided to preferred type one (I) and type two (II) N-acetyllactosamine structures comprising oligosaccharide core sequence Galβ1-3/4GlcNAc structures analogously to T4,

[Mα]ₘGalβ1-3/4[Nα]ₙGlcNAc, Formula T8:

Wherein the variables are as described for T5.

The preferred structures can be divided to preferred Galβ1-3 structures analogously to T8,

[Mα]ₘGalβ1-3[Nα]ₙGlcNAc Formula T9:

Wherein the variables are as described for T5.

These are preferred type I N-acetyllactosamine structures. The invention revealed that the these are very useful for recognition of specific subtypes of the embryonic type stem cells or differentiated variants thereof (tissue type specifically differentiated embryonic type stem cells or various stages of embryonic stem cells). It is notable that various fucosyl- and or sialic acid modification created characteristic pattern for the stem cell type.

The preferred structures can be divided to preferred Galβ1-4GlcNAc core sequence comprising structures analogously to T8,

[Mα]ₘGalβ1-4[Nα]ₙGlcNAc Formula T10:

Wherein the variables are as described for T5.

These are preferred type II N-acetyllactosamine structures. The invention revealed that the these are very useful for recognition of specific subtypes of embryonic type stem cells or differentiated variants thereof (tissue type specifically differentiated embryonic type stem cells or various stages of embryonic stem cells).

It is notable that various fucosyl- and or sialic acid modificationally N-acetyllactosamine structures create especially characteristic pattern for the stem cell type. The invention is further directed to use of combinations binder reagents recognizing at least two different type I and type II acetyllactosamines including at least one fucosylated or sialylated variant and more preferably at least two fucosylated variants or two sialylated variants

Preferred Structures Comprising Terminal Fucα2/3/4-Structures

The invention is further directed to use of combinations binder reagents recognizing:
a) type I and type II acetyllactosamines and their fucosylated variants, and in a preferred embodiment
b) non-sialylated fucosylated and even more preferably
c) fucosylated type I and type II N-acetyllactosamine structures preferably comprising Fucα2-terminal and/or Fucα3/4-branch structure and even more preferably
d) fucosylated type I and type II N-acetyllactosamine structures preferably comprising Fucα2-terminal
for the methods according to the invention of various stem cells especially embryonic type and differentiated variants thereof.

Preferred subgroups of Fucα2-structures includes monofucosylated H type and H type II structures, and difucosylated Lewis b and Lewis y structures.

Preferred subgroups of Fucα3/4-structures includes monofucosylated Lewis a and Lewis x structures, sialylated sialyl-Lewis a and sialyl-Lewis x-structures and difucosylated Lewis b and Lewis y structures.

Preferred type II N-acetyllactosamine subgroups of Fucα3-structures includes monofucosylated Lewis x structures, and sialyl-Lewis x-structures and Lewis y structures.

Preferred type I N-acetyllactosamine subgroups of Fucα4-structures includes monofucosylated Lewis a sialyl-Lewis a and difucosylated Lewis b structures.

The invention is further directed to use of at least two differently fucosylated type one and or and two N-acetyllactosamine structures preferably selected from the group monofucosylated or at least two difucosylated, or at least one monofucosylated and one difucosylated structures.

The invention is further directed to use of combinations binder reagents recognizing fucosylated type I and type II N-acetyllactosamine structures together with binders recognizing other terminal structures comprising Fucα2/3/4-comprising structures, preferably Fucα2-terminal structures, preferably comprising Fucα2Galβ3GalNAc-terminal, more preferably Fucα2Galβ3GalNAcα/β and in especially preferred embodiment antibodies recognizing Fucα2Galβ3GalNAcβ—preferably in terminal structure of Globo- or isoglobotype structures.

Preferred Globo- and Ganglio Core Type-Structures

The invention is further directed to general formula comprising globo and gangliotype Glycan core structures according to formula

[M]ₘGalβ1-x[Nα]ₙHex(NAc)ₚ, wherein m, n and p are integers 0, or 1, independently Formula T11

Hex is Gal or Glc, X is linkage position;

M and N are monosaccharide residues being independently nothing (free hydroxyl groups at the positions) and/or

SAα which is Sialic acid linked to 3-position of Gal or/and 6-position of HexNAc

Galα linked to 3 or 4-position of Gal, or

GalNAcβ linked to 4-position of Gal and/or

Fuc (L-fucose) residue linked to 2-position of Gal

and/or 3 or 4 position of HexNAc, when Gal is linked to the other position (4 or 3),

and HexNAc is GlcNAc, or 3-position of Glc when Gal is linked to the other position (3),

with the provision that sum of m and n is 2

preferably m and n are 0 or 1, independently, and

with the provision that when M is Galα then there is no sialic acid linked to Galβ1, and n is 0 and preferably x is 4.

with the provision that when M is GalNAcβ, then there is no sialic acid α6-linked to Galβ1, and n is 0 and x is 4.

The invention is further directed to general formula comprising globo and gangliotype Glycan core structures according to formula

[M][SAα3]ₙGalβ1-4Glc(NAc)ₚ, Formula T12

wherein n and p are integers 0, or 1, independently

M is Galα linked to 3 or 4-position of Gal, or GalNAcβ linked to 4-position of Gal

and/or SAα is Sialic acid branch linked to 3-position of Gal

with the provision that when M is Galα then there is no sialic acid linked to Galβ1 (n is 0).

The invention is further directed to general formula comprising globo and gangliotype Glycan core structures according to formula

[M][SAα]ₙGalβ1-4Glc, Formula T13

wherein n and p are integer 0, or 1, independently

M is Galα linked to 3 or 4-position of Gal, or

GalNAcβ linked to 4-position of Gal and/or

SAαwhich is Sialic acid linked to 3-position of Gal

with the provision that when M is Galα then there is no sialic acid linked to Galβ1 (n is 0).

The invention is further directed to general formula comprising globo type Glycan core structures according to formula

Galα3/4Galβ1-4Glc. Formula T14

The preferred Globo-type structures includes Galα3/4Galβ1-4Glc, GalNAcβ3Galα3/4Galβ4Glc, Galα4Galβ4Glc (globotriose, Gb3), Galα3Galβ4Glc (isoglobotriose), GalNAcβ3Galα4Galβ4Glc (globotetraose, Gb4 (or G14)), and Fucα2Galβ3GalNAcβ3Galα3/4Galβ4Glc. or when the binder is not used in context of non-differentiated embryonal stem cells or the binder is used together with another preferred binder according to the invention, preferably an other globo-type binder the preferred binder targets further includes Galβ3GalNAcβ3Galα4Galβ4Glc (SSEA-3 antigen) and/or

NeuAcα3Galβ3GalNAcβ3Galα4Galβ4Glc (SSEA-4 antigen) or terminal non-reducing end di or trisaccharide epitopes thereof.

The preferred globotetraosylceramide antibodies does not recognize non-reducing end elongated variants of GalNAcβ3Galα4Galβ4Glc. The antibody in the examples has such specificity as

The invention is further directed to binders for specific epitopes of the longer oligosaccharide sequences including preferably NeuAcα3Galβ3GalNAc, NeuAcα3Galβ3GalNAcβ, NeuAcα3Galβ3GalNAcβ3Galα4Gal when these are not linked to glycolipids and novel fucosylated target structures:

Fucα2Galβ3GalNAcβ3Galα3/4Gal, Fucα2Galβ3GalNAcβ3Galα, Fucα2Galβ3GalNAcβ3Gal, Fucα2Galβ3GalNAcβ3, and Fucα2Galβ3GalNAc.

The invention is further directed to general formula comprising globo and gangliotype Glycan core structures according to formula

[GalNAcβ4][SAα]ₙGalβ1-4Glc, wherein n and p are integer 0, or 1, independently GalNAcβ linked to 4-position of Gal and/or SAα which is Sialic acid branch linked to 3-position of Gal. Formula T15

The preferred Ganglio-type structures includes GalNAcβ4Galβ1-4Glc, GalNAcβ4[SAα3]Galβ1-4Glc, and Galβ3GalNAcβ4[SAα3]Galβ1-4Glc.

The preferred binder target structures further include glycolipid and possible glycoprotein conjugates of of the preferred oligosaccharide sequences. The preferred binders preferably specifically recognizes at least di- or trisaccharide epitope

GalNAcα-Structures

The invention is further directed to recognition of peptide/protein linked GalNAcα-structures according to the Formula T16:[SAα6]ₘGalNAcα[Ser/Thr]ₙ-[Peptide]ₚ, wherein m, n and p are integers 0 or 1, independently,

wherein SA is sialic acid preferably NeuAc,Ser/Thr indicates linking serine or threonine residues,

Peptide indicates part of peptide sequence close to linking residue,

with the provision that either m or n is 1.

Ser/Thr and/or Peptide are optionally at least partially necessary for recognition for the binding by the binder. It is realized that when Peptide is included in the specificity, the antibody have high specificity involving part of a protein structure. The preferred antigen sequences of sialyl-Tn: SAα6GalNAcα, SAα6GalNAcαSer/Thr, and SAα6GalNAcαSer/Thr-Peptide and Tn-antigen: GalNAcαSer/Thr, and GalNAcαSer/Thr-Peptide. The invention is further directed to the use of combinations of the GalNAcα-structures and combination of at least one GalNAcα-structure with other preferred structures.

Combinations of Preferred Binder Groups

The present invention is especially directed to combined use of at least a) fucosylated, preferably α2/3/4-fucosylated structures and/or b) globo-type structures and/or c) GalNAcα-type structures. It is realized that using a combination of binders recognizing structures involving different biosynthesis and thus having characteristic binding profile with a stem cell population. More preferably at least one binder for a fucosylated structure and and globostructures, or fucosylated structure and GalNAcα-type structure is used, most preferably fucosylated structure and globostructure are used.

Fucosylated and Non-Modified Structures

The invention is further directed to the core disaccharide epitope structures when the structures are not modified by sialic acid (none of the R-groups according to the Formulas T1-T3 or M or N in formulas T4-T7 is not sialic acid.

The invention is in a preferred embodiment directed to structures, which comprise at least one fucose residue according to the invention. These structures are novel specific fucosylated terminal epitopes, useful for the analysis of stem cells according to the invention. Preferably native stem cells are analyzed.

The preferred fucosylated structures include novel α3/4fucosylated markers of human stem cells such as (SAα3)₀ₒᵣ₁Galβ3/4(Fucα4/3)GlcNAc including Lewis x and and sialylated variants thereof.

Among the structures comprising terminal Fucα1-2 the invention revealed especially useful novel marker structures comprising Fucα2Galβ3GalNAcα/β and Fucα2Galβ3(Fucα4)₀ₒᵣ₁GlcNAcβ, these were found useful studying embryonic stem cells. A especially preferred antibody/binder group among this group is antibodies specific for Fucα2Galβ3GlcNAcβ, preferred for high stem cell specificity. Another preferred structural group includes Fucα2Gal comprising glycolipids revealed to form specific structural group, especially interesting structure is globo-H-type structure and glycolipids with terminal Fucα2Galβ3GalNAcβ, preferred with interesting biosynthetic context to earlier speculated stem cell markers.

Among the antibodies recognizing Fucα2Galβ4GlcNAcβ substantial variation in binding was revealed likely based on the carrier structures, the invention is especially directed to antibodies recognizing this type of structures, when the specificity of the antibody is similar to the ones binding to the embryonic stem cells as shown in Example 18 with fucose recognizing antibodies.

The invention is preferably directed to antibodies recognizing Fucα2Galβ4GlcNAcβ on N-glycans, revealed as common structural type in terminal epitope Table 21. In a separate embodiment the antibody of the non-binding clone is directed to the recognition of the feeder cells.

The preferred non-modified structures includes Galβ4Glc, Galβ3GlcNAc, Galβ3GalNAc, Galβ4GlcNAc, Galβ3GlcNAcβ, Galβ3GalNAcβ/α, and Galβ4GlcNAcβ. These are preferred novel core markers characteristics for the various stem cells. The structure Galβ3GlcNAc is especially preferred as novel marker observable in hESC cells. Preferably the structure is carried by a glycolipid core structure according to the invention or it is present on an O-glycan. The non-modified markers are preferred for the use in combination with at least one fucosylated or/and sialylated structure for analysis of cell status.

Additional preferred non-modified structures includes GalNAcβ-structures includes terminal LacdiNAc, GalNAcβ4GlcNAc, preferred on N-glycans and GalNAcβ3Gal GalNAcβ3Gal present in globoseries glycolipids as terminal of globotetraose structures.

Among these characteristic subgroup of Gal(NAc)β3-comprising Galβ3GlcNAc, Galβ3GalNAc, Galβ3GlcNAcβ, Galβ3GalNAcβ/α, and GalNAcβ3Gal GalNAcβ3Gal and the characteristic subgroup of Gal(NAc)β4-comprising Galβ4Glc, Galβ4GlcNAc, and Galβ4GlcNAc are separately preferred.

Preferred Sialylated Structures

The preferred sialylated structures includes characteristic SAα3Galβ-structures SAα3Galβ4Glc, SAα3Galβ3GlcNAc, SAα3Galβ3GalNAc, SAα3Galβ4GlcNAc, SAα3Galβ3GlcNAcβ, SAα3Galβ3GalNAcβ/α, and SAα3Galβ4GlcNAcβ; and biosynthetically partially competing SAα6Galβ-structures SAα6Galβ4Glc, SAα6Galβ4Glcβ; SAα6Galβ4GlcNAc and Sα6Galβ4GlcNAcβ; and disialo structures SAα3Galβ3(SAα6)GalNAcβ/α,

The invention is preferably directed to specific subgroup of Gal(NAc)β3-comprising SAα3Galβ3GlcNAc, SAα3Galβ3GalNAc, SAα3Galβ4GlcNAc, SAα3Galβ3GlcNAcβ, SAα3Galβ3GalNAcβ/α and SAα3Galβ3(SAα6)GalNAcβ/α, and Gal(NAc)β4-comprising sialylated structures. SAα3Galβ4Glc, and SAα3Galβ4GlcNAcβ; and SAα6Galβ4Glc, SAα6Galβ4Glcβ; SAα6Galβ4GlcNAc and SAα6Galβ4GlcNAcβ

These are preferred novel regulated markers characteristics for the various stem cells.

Use Together with a Terminal ManαMan-Structure

The terminal non-modified or modified epitopes are in preferred embodiment used together with at least one ManαMan-structure. This is preferred because the structure is in different N-glycan or glycan subgroup than the other epitopes.

Core Structures of the Terminal Epitopes

It is realized that the target epitope structures are most effectively recognized on specific N-glycans, O-glycan, or on glycolipid core structures.

Elongated Epitopes—Next Monosaccharide/Structure on the Reducing End of the Epitope

The invention is especially directed to optimized binders and production thereof, when the binding epitope of the binder includes the next linkage structure and even more preferably at least part of the next structure (monosaccharide or amino acid for O-glycans or ceramide for glycolipid) on the reducing side of the target epitope. The invention has revealed the core structures for the terminal epitopes as shown in the Examples and ones summarized in Table 21.

It is realized that antibodies with longer binding epitopes have higher specificity and thus will recognize that desired cells or cell derived components more effectively. In a preferred embodiment the antibodies for elongated epitopes are selected for effective analysis of embryonic type stem cells.

The invention is especially directed to the methods of antibody selection and optionally further purification of novel antibodies or other binders using the elongated epitopes according to the invention. The preferred selection is performed by contacting the glycan structure (synthetic or isolated natural glycan with the specific sequence) with a serum or an antibody or an antibody library, such as a phage display library. Data about these methods are well known in the art and available from internet for example by searching pubmed-medical literature database (www.ncbi.nlm.nih.gov/entrez) or patents e.g. in espacenet (fi.espacenet.com). The specific antibodies are especially preferred for the use of the optimized recognition of the glycan type specific terminal structures as shown in the Examples and ones summarized in the Table 21.

It is further realized that part of the antibodies according to the invention and shown in the examples have specificity for the elongated epitopes. The inventors found out that for example Lewis x epitope can be recognized on N-glycan by certain terminal Lewis x specific antibodies, but not so effectively or at all by antibodies recognizing Lewis xβ1-3Gal present on poly-N-acetyllactosamines or neolactoseries glycolipids.

N-Glycans

The invention is especially directed to recognition of terminal N-glycan epitopes on biantennary N-glycans. The preferred non-reducing end monosaccharide epitope for N-glycans comprise β2Man and its reducing end further elongated variants

β2Man, β2Manα, β2Manα3, and β2Manα6

The invention is especially directed to recognition of lewis x on N-glycan by N-glycan Lewis x specific antibody described by Ajit Varki and colleagues Glycobiology (2006) Abstracts of Glycobiology society meeting 2006 Los Angeles, with possible implication for neuronal cells, which are not directed (but disclaimed) with this type of antibody by the present invention. Invention is further directed to antibodies with specificity of type 2 N-acetyllactosamineβ2Man recognizing biantennary N-glycan directed antibody as described in Ozawa H et al (1997) Arch Biochem Biophys 342, 48-57.

O-Glycans, Reducing End Elongated Epitopes

The invention is especially directed to recognition of terminal O-glycan epitopes as terminal core I epitopes and as elongated variants of core I and core II O-glycans.

The preferred non-reducing end monosaccharide epitope for O-glycans comprise:

a) Core I epitopes linked to αSer/Thr-[Peptide]₀₋₁,

wherein Peptide indicates peptide which is either present or absent. The invention is preferably

b) Preferred core II-type epitopes

R1β6[R2β3Galβ3]ₙGalNAcαSer/Thr, wherein n is = or 1 indicating possible branch in the structure and R1 and R2 are preferred positions of the terminal epitopes, R1 is more preferred

c) Elongated Core I epitope

β3Gal and its reducing end further elongated variants β3Galβ3GalNAcα, β3Galβ3GalNAcαSer/Thr

O-glycan core I specific and ganglio/globotype core reducing end epitopes have been described in (Saito S et al. J Biol Chem (1994) 269, 5644-52), the invention is preferably directed to similar specific recognition of the epitopes according to the invention.

O-glycan core II sialyl-Lewis x specific antibody has been described in Walcheck B et al. Blood (2002) 99, 4063-69.

Peptide specificity including antibodies for recognition of O-glycans includes mucin specific antibodies further recognizing GalNAcalfa (Tn) or Galb3GalNAcalfa (T/TF) structures (Hanisch F-G et al (1995) cancer Res. 55, 4036-40; Karsten U et al. Glycobiology (2004) 14, 681-92;

Glycolipid Core Structures

The invention is furthermore directed to the recognition of the structures on lipid structures. The preferred lipid core structures include:
a) βCer (ceramide) for Galβ4Glc and its fucosyl or sialyl derivatives
b) β3/6Gal for type I and type II N-acetyllactosamines on lactosyl Cer-glycolipids, preferred elongated variants includes β3/6[Rβ6/3]ₙGalβ, β3/6[Rβ6/3]ₙGalβ4 and β3/6[Rβ6/3]ₙGalβ4Glc, which may be further branched by another lactosamine residue which may be partially recognized as larger epitope and n is 0 or 1 indicating the branch, and R1 and R2 are preferred positions of the terminal epitopes. Preferred linear (non-branched) common structures include β3Gal, β3Galβ, β3Galβ4 and β3Galβ4Glc
c) α3/4Gal, for globoseries epitopes, and elongated variants α3/4Galβ, α3/4Galβ4Glc preferred globoepitopes have elongated epitopes α4Gal, α4Galβ, α4Galβ4Glc, and preferred isogloboepitopes have elongated epitopes α3Gal, α3Galβ, α3Galβ4Glc
d) β4Gal for ganglio-series epitopes comprising, and preferred elongated variants include β4Galβ, and β4Galβ4Glc

O-glycan core specific and ganglio/globotype core reducing end epitopes have been described in (Saito S et al. J Biol Chem (1994) 269, 5644-52), the invention is preferably directed to similar specific recognition of the epitopes according to the invention.

Poly-N-acetyllactosamines

Poly-N-acetyllactosamine backbone structures on O-glycans, N-glycans, or glycolipids comprise characteristic structures similar to lactosyl(cer) core structures on type I (lactoseries) and type II (neolacto) glycolipids, but terminal epitopes are linked to another type I or type II N-acetyllactosamine, which may from a branched structure. Preferred elongated epitopes include: β3/6Gal for type I and type II N-acetyllactosamines epitope, preferred elongated variants includes R1β3/6[R2β6/3]ₙGalβ, R1β3/6[R2β6/3]ₙGalβ3/4 and R1β3/6[R2β6/3]ₙGalβ3/4GlcNAc, which may be further branched by another lactosamine residue which may be partially recognized as larger epitope and n is 0 or 1 indicating the branch, and R1 and R2 are preferred positions of the terminal epitopes. Preferred linear (non-branched) common structures include β3Gal, β3Galβ, β3Galβ4 and β3Galβ4GlcNAc.

Numerous antibodies are known for linear (i-antigen) and branched poly-N-acetyllactosamines (I-antigen), the invention is further directed to the use of the lectin PWA for recognition of I-antigens. The inventors revealed that poly-N-acetyllactosamines are characteristic structures for specific types of human stem cells. Another preferred binding regent, enzyme endo-beta-galactosidase was used for characterization poly-N-acetyllactosamines on glycolipids and on glycoprotein of the stem cells. The enzyme revealed characteristic expression of both linear and branched poly-N-acetyllactosamine, which further comprised specific terminal modifications such as fucosylation and/or sialylation according to the invention on specific types of stem cells.

Combinations of Elongated Core Epitopes

It is realized that stronger labeling may be obtained if the same terminal epitope is recognized by antibody binding to target structure present on two or three of the major carrier types O-glycans, N-glycans and glycolipids. It is further realized that in context of such use the terminal epitope must be specific enough in comparison to the epitopes present on possible contaminating cells or cell materials. It is further realized that there is highly terminally specific antibodies, which allow binding to on several elongation structures.

The invention revealed each elongated binder type useful in context of stem cells. Thus the invention is directed to the binders recognizing the terminal structure on one or several of the elongating structures according to the invention

Preferred Group of Monosaccharide Elongation Structures

The invention is directed to use of binders with elongated specificity, when the binders recognize or is able to bind at least one reducing end elongation monosaccharide epitope according to the formula

AxHex(NAc)ₙ, wherein A is anomeric structure alfa or beta, X is linkage position 2, 3,4, or 6

And Hex is hexopyranosyl residue Gal, or Man, and n is integer being 0 or 1, with the provisions that when n is 1 then AxHexNAc is β6GalNAc, when Hex is Man, then AxHex is β2Man, and when Hex is Gal, then AxHex is β3Gal or β6Gal.

Beside the monosaccharide elongation structures αSer/Thr are preferred reducing end elongation structures for reducing end GalNAc-comprising O-glycans and βCer is preferred for lactosyl comprising glycolipid epitopes.

The invention is directed to the preferred terminal epitopes according to the invention comprising the preferred reducing end elongation of the N-acetyllactosamine epitomes described in Formulas T1-T11, referred as T1E-T11E in elongated form

A preferred example is

[Mα]ₘGalβ1-3/4[Nα]ₙGlcNAcAxHex(NAc)ₙ Formula T8E:

wherein

wherein m, n and p are integers 0, or 1, independently

Hex is Gal or Glc,

X is linkage position

M and N are monosaccharide residues being independently nothing (free hydroxyl groups at the positions) and/or

SA which is Sialic acid linked to 3-position of Gal or/and 6-position of HexNAc and/or

Fuc (L-fucose) residue linked to 2-position of Gal

and/or 3 or 4 position of GlcNAc, when Gal is linked to the other position (4 or 3),

and HexNAc is GlcNAc, or 3-position of Glc when Gal is linked to the other position (3),

with the provision that sum of m and n is 2

preferably m and n are 0 or 1, independently.

A is anomeric structure alfa or beta, X is linkage position 2, 3,or 6

And Hex is hexopyranosyl residue Gal, or Man, and n is integer being 0 or 1, with the provisions that when n is 1 then AxHexNAc is β6GalNAc, when Hex is Man, then AxHex is β2Man, and when Hex is Gal, then AxHex is β3Gal or β6Gal.

The most preferred structures are according to the formula

Formula T8E beta, wherein the anomeric structure is beta:

[Mα]ₘGalβ1-3/4[Nα]ₙGlcNAcβxHex(NAc)ₙ

A preferred group of type II Lactosamines are β2-linked on Man or N-glycans or β6-linked on Gal(NAc) in O-glycan/poly-LacNac structures according to the

[Mα]ₘGalβ1-4[Nα]ₙGlcNAcAxHex(NAc)ₙ Formula T10E

[Mα]ₘGalβ1-4[Na]ₙGlcNAcβ2Man Formula T10EMan:

and

[Mα]ₘGalβ1-4[Nα]ₙGlcNAcβ6Gal(NAc) Formula T10EGal(NAc):

and further elongated structures according to the invention.

A preferred group of type I Lactosamines are β3—on Gal

According to the Formula T9E

[Mα]ₘGalβ1-3[Nα]ₙGlcNAcβ3Gal

The preferred subgroups of the elongation structures includes i) similar structural epitopes present on O-glycans, polylactosamine and glycolipid cores: β3/6Gal or β6GalNAc; with preferred further subgroups ia) β6GalNAc/β6Gal and ib) β3Gal; ii) N-glycan type epitope β2Man; and iii) globoseries epitopes α3Gal or α4Gal. The groups are preferred for structural similarity on possible cross reactivity within the groups, which can be used for increasing labeling intensity when background materials are controlled to be devoid of the elongated structure types.

Useful binder specificities including lectin and elongated antibody epitopes is available from reviews and monographs such as (Debaray and Montreuil (1991) Adv. Lectin Res 4, 51-96; “The molecular immunology of complex carbohydrates” Adv Exp Med Biol (2001) 491 (ed Albert M Wu) Kluwer Academic/Plenum publishers, New York; “Lectins” second Edition (2003) (eds Sharon, Nathan and Lis, Halina) Kluwer Academic publishers Dordrecht, The Netherlands and internet databases such as pubmed/espacenet or antibody databases such as www.glyco.is.ritsumei.ac.jp/epitope/, which list monoclonal antibody glycan specificities).

Combination of the Preferred Elongated Epitopes

The invention is directed in a preferred embodiment combined use of the preferred structures and elongated structures for recognition of stem cells. In a preferred embodiment at least one type I LacNAc or type II lacNAc structure are used, in another preferred embodiment a non-reducing end non-modified LacNAc is used with α2Fucosylated LacNAc, Lewis x or sialylated LacNAc, in a preferred embodiment α2Fucosylated type I and type II LacNAc are used. The inventors used factor analysis to produce more preferred combinations according to the invention including use of complex type glycans together with high mannose or Low mannose glycan. In a preferred embodiment a LacNAc structure is used together with a preferred glycolipid structure, preferably globotriose type. The invention is preferably directed to recognition of differentiation and/or cell culture condition associated changes in the stem cells.

Preferred Elongated Epitopes

It is realized that elongated glycan epitopes are useful for recognition of the embryonic type stem cells according to the invention. The invention is directed to the use of -some of the structures for characterizing all the cell types, while certain structural motifs are more common at a specific differentiation stage.

It is further realized that some of the terminal structures are expressed at especially high levels and thus especially useful for the recognition of one or several types of cells.

The terminal epitopes and the glycan types are listed in Table 21, based on the structural analysis of the glycan types following preferred elongated structural epitopes that are preferred as novel markers for embryonal type stem cells and for the uses according to the invention.

Preferred Terminal Galβ3/4 Structures

Type II N-Acetyllactosamine Based Structures

Terminal Type II N-Acetyllactosamine Structures

The invention revealed preferred type II N-acetyllactosamines including specific O-glycan, N-glycan and glycolipid epitopes. The invention is in a preferred embodiment especially directed to abundant O-glycan and N-glycan epitopes. The invention is further directed to the recognition of a characteristic glycolipid type II LacNAc terminal. The invention is especially directed to the use of the Type II LacNAc for recognition of non-differentiated embryonal type stem cells (stage I) and similar cells or for the analysis of the differentiation stage. It is however realized that substantial amounts of the structures are present in the more differentiated cells as well.

Elongated type II LacNAc structures are especially expressed on N-glycans. Preferred type II LacNAc structures are β2-linked to the biantennary N-glycan core structure, including the preferred epitopes Galβ4GlcNAcβ2Man, Galβ4GlcNAcβ2Manα, Galβ4GlcNAcβ2Manα3/6Man and Galβ4GlcNAcβ2Manα3/6Manβ4

The invention further revealed novel O-glycan epitopes with terminal type II N-acetyllactosamine structures expressed effectively on the embryonal type cells. The analysis of the O-glycan structures revealed especially core II N-acetyllactosamines with the terminal structure. The preferred elongated type II N-acetyllactosamines thus includes Galβ4GlcNAcβ6GalNAc, Galβ4GlcNAcβ6GalNAcα, Galβ4GlcNAcβ36(Galβ33)GalNAc, and Galβ4GlcNAcβ6(Galβ3)GalNAcα.

The invention further revealed the presence of type II LacNAc on glycolipids. The present invention reveals for the first time terminal type II N-acetyllactosamine on glycolipids of stem cells. The neolacto glycolipid family is an important glycolipid family characteristically expressed on certain tissues but not on others.

The preferred glycolipid structures include epitopes, preferably non-reducing end terminal epitopes of linear neolactotetraosyl ceramide and elongated variants thereof Galβ4GlcNAcβ3Gal, Galβ4GlcNAcβ3Galβ4,Galβ4GlcNAcβ3Galβ4Glc(NAc), Galβ4GlcNAcβ3Galβ4Glc, and Galβ4GlcNAcβ3Galβ4GlcNAc. It is further realized that specific reagents recognizing the linear polylactosamines can be used for the recognition of the structures, when these are linked to protein linked glycans. In a preferred embodiment the invention is directed to the poly-N-acetyllactosamines linked to N-glycans, preferably β2-linked structures such as Galβ4GlcNAcβ3Galβ4GlcNAcβ2Man on N-glycans. The invention is further directed to the characterization of the poly-N-acetyllactosamine structures of the preferred cells and their modification by SAα3, SAα6, Fucα2 to non-reducing end Gal and by Fucα3 to GlcNAc residues.

The invention is preferably directed to recognition of tetrasaccharides, hexasaccharides, and octasaccharides. The invention further revealed branched glycolipid polylactosamines including terminal type II LacNAc epitopes, preferably these include Galβ4GlcNAcβ6Gal, Galβ4GlcNAcβ6Galβ, Galβ4GlcNAcβ6(Galβ4GlcNAcβ3)Gal, and Galβ4GlcNAcβ6(Galβ4GlcNAcβ3)Galβ3,Galβ4GlcNAcβ6(Galβ4GlcNAcβ3)Galβ4Glc(NAc), Galβ4GlcNAcβ6(Galβ4GlcNAcβ3)Galβ4Glc, and Galβ4GlcNAcβ6(Galβ4GlcNAcβ3)Galβ4GlcNAc.

It is realized that antibodies specifically binding to the linear or branched poly-N-acetyllactosamines are well known in the art. The invention is further directed to reagents recognizing both branched polyLacNAcs and core II O-glycans with similar β6Gal(NAc) epitopes.

Lewis x Structures

Elongated Lewis x structures are especially expressed on N-glycans. Preferred Lewis x structures are β2-linked to the biantennary N-glycan core structure, including the preferred structures Galβ4(Fucα3)GlcNAcβ2Man, Galβ4(Fucα3)GlcNAcβ2Manα, Galβ4(Fucα3)GlcNAcβ2Manα3/6Man, Galβ4(Fucα3)GlcNAcβ2Manα3/6Manβ4

The invention further revealed the presence of Lewis x on glycolipids. The preferred glycolipid structures include Gal(Fucα3)β4GlcNAcβ3Gal, Galβ4(Fucα3)GlcNAcβ3Gal, Galβ4(Fucα3)GlcNAcβ3Galβ4, Galβ4(Fucα3)GlcNAcβ3Galβ4Glc(NAc), Galβ4(Fucα3)GlcNAcβ3Galβ4Glc, and Galβ4(Fucα3)GlcNAcβ3Galβ4GlcNAc.

The invention further revealed the presence of Lewis x on O-glycans. The preferred O-glycan structures include preferably the core II structures Galβ4(Fucα3)GlcNAcβ6GalNAc, Galβ4(Fucα3)GlcNAcβ6GalNAcα, Galβ4(Fucα3)GlcNAcβ6(Galβ3)GalNAc, and Galβ4(Fucα3)GlcNAcβ6(Galβ3)GalNAcα.

H Type II Structures

Specific elongated H type II structural epitopes are especially expressed on N-glycans. Preferred H type II structures are β2-linked to the biantennary N-glycan core structure, Fucα2Galβ4GlcNAcβ2Manα3/6Manβ4

The invention further revealed the presence of H type II on glycolipids. The preferred glycolipid structures includes Fucα2Galβ4GlcNAcβ3Gal, Fucα2Galβ4GlcNAcβ3Gal, Fucα2Galβ4GlcNAcβ3Galβ4, Fucα2Galβ4GlcNAcβ3Galβ4Glc(NAc), Fucα2Galβ4GlcNAcβ3Galβ4Glc, and Fucα2Galβ4GlcNAcβ3Galβ4GlcNAc.

The invention further revealed the presence of H type II on O-glycans. The preferred O-glycan structures include preferably core II structures Fucα2Galβ4GlcNAcβ6GalNAc, Fucα2Galβ4GlcNAcβ6GalNAcα, Fucα2Galβ4GlcNAcβ6(Galβ3)GalNAc, and Fucα2Galβ4GlcNAcβ6(Galβ3)GalNAcα.

Sialylated Type II N-Acetyllactosamine Structures

The invention revealed preferred sialylated type II N-acetyllactosamines including specific O-glycan, N-glycan and glycolipid epitopes. The invention is in a preferred embodiment especially directed to abundant O-glycan and N-glycan epitopes. SA refers here to sialic acid, preferably Neu5Ac or Neu5Gc, more preferably Neu5Ac. The sialic acid residues are SAα3Gal or SAα6Gal, it is realized that these structures when presented as specific elongated epitopes form characteristic terminal structures on glycans.

Sialylated type II LacNAc structural epitopes are especially expressed on N-glycans. Preferred type II LacNAc structures are β2-linked to biantennary N-glycan core structure, including the preferred terminal epitopes SAα3/6Galβ4GlcNAcβ2Man, SAα3/6Galβ4GlcNAcβ2Manα, and SAα3/6Galβ4GlcNAcβ2Manα3/6Manβ4. The invention is directed to both SAα3-structures (SAα3Galβ4GlcNAcβ2Man, SAα3Galβ4GlcNAcβ2Manα, and SAα3Galβ4GlcNAcβ2Manα3/6Manβ4) and SAα6-epitopes (SAα6Galβ4GlcNAcβ2Man, SAα6Galβ4GlcNAcβ2Manα, and SAα6Galβ4GlcNAcβ2Manα3/6Manβ4) on N-glycans.

The SAα3-N-glycan epitopes are preferred for the analysis of the non-differentiated stage I embryonic type cells. The SAα6-N-glycan epitopes are preferred for analysis of the differentiated/or differentiating embryonic type cells, such as embryoid bodies and stage III differentiated embryonic type cells. It is realized that the combined analysis of both types of N-glycans is useful for the characterization of the embryonic type stem cells.

The invention further revealed novel O-glycan epitopes with terminal sialylated type II N-acetyllactosamine structures expressed effectively on the embryonal type cells. The analysis of O-glycan structures revealed especially core II N-acetyllactosamines with the terminal structure. The preferred elongated type II sialylated N-acetyllactosamines thus include SAα3/6Galβ4GlcNAcβ6GalNAc, SAα3/6Galβ4GlcNAcβ6GalNAcα, SAα3/6Galβ4GlcNAcβ6(Galβ3)GalNAc, and SAα3/6Galβ4GlcNAcβ6(Galβ3)GalNAcα. The SAα3-structures were revealed as preferred structures in context of the O-glycans including SAα3Galβ4GlcNAcβ6GalNAc, SAα3Galβ4GlcNAcβ6GalNAcα, SAα3Galβ4GlcNAcβ6(Galβ3)GalNAc, and SAα3Galβ4GlcNAcβ6(Galβ3)GalNAcα.

Specific Preferred Tetrasaccharide Type II Lactosamine Epitopes

It is realized that highly effective reagents can in a preferred embodiment recognize epitopes which are larger than a trisaccharide. Therefore the invention is further directed to the branched terminal type II lactosamine derivatives Lewis y Fucα2Galβ4(Fucα3)GlcNAc and sialyl-Lewis x SAα3Galβ4(Fucα3)GlcNAc as preferred elongated or large glycan structural epitopes. It is realized that the structures are combinations of preferred terminal trisaccharide sialyl-lactosamine, H-type II and Lewis x epitopes. The analysis of the epitopes is preferred as additionally useful method in the context of analysis of other terminal type II epitopes. The invention is especially directed to—further defining the core structures carrying the Lewis y and sialyl-Lewis x epitopes on various types of glycans and optimizing the recognition of the structures by including the recognition of the preferred glycan core structures.

Structures Analogous to the Type II Lactosamines

The invention is further directed to the recognition of elongated epitopes analogous to the type II N-acetyllactosamines including LacdiNAc especially on N-glycans and lactosylceramide (Galβ4GlcβCer) glycolipid structure. These share similarity with LacNAc the only difference being the number of NAc residues on the monosaccharide residues.

LacdiNAc Structures

It is realized that LacdiNac is relatively rare and characteristic glycan structure and it is therefore especially preferred for the characterization of the embryonic type cells. The invention revealed the presence of LacdiNAc on N-glycans at least as β2-linked terminal epitope. The structures were characterized by specific glycosidase cleavages. The LacdiNAc structures have same mass as structures with two terminal GlcNAc containing structures in structural Table 13, Table 13 includes representative structures indicating only single isomeric structures for a specific mass number. The preferred elongated LacdiNAc epitopes thus includes GalNAcβ4GlcNAcβ2Man, GalNAcβ4GlcNAcβ2Manα, and GalNAcβ4GlcNAcβ2Manα3/6Manβ4. The invention further revealed fucosylation of LacdiNAc containing glycan structures and the preferred epitopes thus further include GalNAcβ4(Fucα3)GlcNAcβ2Man, GalNAcβ4(Fucα3)GlcNAcβ2Manα, GalNAcβ4(Fucα3)GlcNAcβ2Manα3/6Manβ4GalNAc(Fucα3)β4GlcNAcβ2Manα3/6Manβ4. It is realized that presence of β6-linked sialic acid of LacNac of structure with mass number 2263, table 13 indicates that at least part of the fucose is present on the LacdiNAc arm of the molecule based on the competing nature of α6-sialylation and α3-fucosylation on enzyme specificity level (alternative assignment presented in the Table 13).

Type I N-Acetyllactosamine Based Structures

Terminal Type I N-Acetyllactosamine Structures

The invention revealed preferred type I N-acetyllactosamines including specific O-glycan, N-glycan and glycolipid epitopes. The invention is in a preferred embodiment especially directed to abundant glycolipid epitopes. The invention is further preferably directed to the recognition of characteristic O-glycan type I LacNAc terminals.

The invention is especially directed to the use of the Type I LacNAc for the recognition of non-differentiated embryonal type stem cells (stage I) and similar cells or for the analysis of the differentiation stage. It is however realized that substantial amount of the structures are present in the more differentiated cells as well.

The invention further revealed novel O-glycan epitopes with terminal type I N-acetyllactosamine structures expressed effectively on the embryonal type cells. The analysis of O-glycan structures revealed especially core II N-acetyllactosamines with the terminal structure on type II lactosamine. The preferred elongated type I N-acetyllactosamines thus includes Galβ3GlcNAcβ3Galβ4GlcNAcβ6GalNAc, Galβ3GlcNAcβ3Galβ4GlcNAcβ6GalNAcα, Galβ3GlcNAcβ3GalGlcNAcβ6(Galβ3)GalNAc, and Galβ3GlcNAcβ3Galβ4GlcNAcβ6(Galβ3)GalNAcα.

The invention further revealed the presence of type I LacNAc on glycolipids. The present invention reveals for the first time terminal type I N-acetyllactosamine on glycolipids. The Lacto glycolipid family is an important glycolipid family characteristically expressed on certain tissue but not on others.

The preferred glycolipid structures include-epitopes, preferably non-reducing end terminal epitopes, of linear lactoteraosyl ceramide and elongated variants thereof Galβ3GlcNAcβ3Gal, Galβ3GlcNAcβ3Galβ4, Galβ3GlcNAcβ3Galβ4Glc(NAc), Galβ3GlcNAcβ3Galβ4Glc, and Galβ3GlcNAcβ3Galβ4GlcNAc. It is further realized that specific reagents recognizing the linear polylactosamines can be used for the recognition of the structures, when these are linked to protein linked glycans. It is especially realized that the terminal tri- and tetrasaccharide epitopes on the preferred O-glycans and glycolipids are essentially the same. The invention is in a preferred embodiment directed to the recognition of the both structures by the same binding reagent such as a monoclonal antibody

The invention is further directed to the characterization of the terminal type I poly-N-acetyllactosamine structures of the preferred cells and their modification by SAα3, Fucα2 to non-reducing end Gal and by SAα6 or Fucα3 to GlcNAc residues and other core glycan structures of the derivatized type I N-acetyllactosamines.

A preferred elongated type I LacNAc structure is expressed on N-glycans. Preferred type I LacNAc structures are β2-linked to the biantennary N-glycan core structure, the preferred epitopes being Galβ3GlcNAcβ2Man, Galβ3GlcNAcβ2Manα and Galβ3GlcNAcβ2Manα3/6Manβ4.

Fucosylated Type I LacNAcs

Lewis a Structures

The invention revealed the presence of Lewis a structures on glycolipids. The invention is further directed to related poly-N-acetyllactosamine structures with similar terminal epitopes. The preferred glycolipid structures includes Galβ3(Fucα4)βGlcNAcβ3Gal, Galβ3(Fucα4)βGlcNAcβ3Gal, Galβ3(Fucα4)βGlcNAcβ3Galβ4, Galβ3(Fucα4)βGlcNAcβ3Galβ4Glc(NAc), Galβ3(Fucα4)βGlcNAcβ3Galβ4Glc, and Galβ3(Fucα4)βGlcNAcβ3Galβ4GlcNAc.

The invention is further directed to the presence of Lewis a on elongated O-glycans. The preferred O-glycan polylactosamine type structures include preferably the core II structures Galβ3(Fucα4)GlcNAcβ3Galβ4GlcNAcβ6GalNAc, Galβ3(Fucα4)GlcNAcβ3Galβ4GlcNAcβ6GalNAcα, Galβ3(Fucα4)GlcNAcβ3Galβ4GlcNAcβ6(Galβ3)GalNAc, and Galβ3(Fucα4)GlcNAcβ3Galβ4GlcNAcβ6(Galβ3)GalNAcα.

H Type I Structures

A Preferred elongated H type I structure is on lacto series glycolipids or related poly-N-acetyllactosamine structures. The preferred glycolipid/polylactosamine structures includes Fucα2Galβ3GlcNAcβ3Gal, Fucα2Galβ3GlcNAcβ3Gal, Fucα2Galβ3GlcNAcβ3Galβ4, Fucα2Galβ3GlcNAcβ3Galβ4Glc(NAc), Fucα2Galβ3GlcNAcβ3Galβ4Glc, and Fucα2Galβ3GlcNAcβ3Galβ4GlcNAc.

The invention is further directed to the presence of H type I on elongated O-glycans. The preferred O-glycan polylactosamine type structures include preferably the core II structures Fucα2Galβ3GlcNAcβ3Galβ4GlcNAcβ6GalNAc, Fucα2Galβ3GlcNAcβ3Galβ4GlcNAcβ6GalNAcα, Fucα2Galβ3GlcNAcβ3Galβ4GlcNAcβ6(Galβ3)GalNAc, and Fucα2Galβ3GlcNAcβ3Galβ4GlcNAcβ6(Galβ3)GalNAcα.

Specific Preferred Tetrasaccharide Type I Lactosamine Epitopes

It is realized that highly effective reagents can in a preferred embodiment recognize epitopes which are larger than a trisaccharide. Therefore the invention is further directed to the branched terminal type I lactosamine derivatives Lewis b Fucα2Galβ3(Fucα4)GlcNAc and sialyl-Lewis a SAα3Galβ3(Fucα4)GlcNAc as preferred elongated or large glycan structural epitopes. It realized that the structures are combinations of preferred terminal trisaccharide sialyl-lactosamine, H-type I and Lewis a epitopes. The analysis of the epitopes is preferred as additionally useful method in the context of analysis of other terminal type I epitopes. The invention is especially directed to-further defining the core structures carrying the type Lewis b and sialyl-Lewis a epitopes on various types of glycans and optimizing the recognition of the structures by including the recognition of preferred glycan core structures. The invention revealed that at least some of the sialyl-Lewis a epitopes are scarce on stage I cells and the structure is associated more with differentiated cell types. As used herein, “binder”, “binding agent” and “marker” are used interchangeably.

Antibodies

Various procedures known in the art may be used for the production of polyclonal antibodies to peptide motifs and regions or fragments thereof. For the production of antibodies, any suitable host animal (including but not limited to rabbits, mice, rats, or hamsters) are immunized by injection with a peptide (immunogenic fragment). Various adjuvants may be used to increase the immunological response, depending on the host species, including but not limited to Freund's (complete and incomplete) adjuvant, mineral gels such as aluminum hydroxide, surface active substances such as lysolecithin, pluronic polyols, polyanions, oil emulsions, keyhole limpet hemocyanins, dinitrophenol, and potentially useful human adjuvants such as BCG {Bacille Calmette-Guerin) and Corynebacterium parvum.

A monoclonal antibody to a peptide or glycan motif(s) may be prepared by using any technique which provides for the production of antibody molecules by continuous cell lines in culture. These include but are not limited to the hybridoma technique originally described by Köhler et al., (Nature, 256: 495-497, 1975), and the more recent human B-cell hybridoma technique (Kosbor et al., Immunology Today, 4: 72, 1983) and the EBV-hybridoma technique (Cole et al., Monoclonal Antibodies and Cancer Therapy, Alan R Liss, Inc., pp. 77-96, 1985), all specifically incorporated herein by reference. Antibodies also may be produced in bacteria from cloned immunoglobulin cDNAs. With the use of the recombinant phage antibody system it may be possible to quickly produce and select antibodies in bacterial cultures and to genetically manipulate their structure.

When the hybridoma technique is employed, myeloma cell lines may be used. Such cell lines suited for use in hybridoma-producing fusion procedures preferably are non-antibody-producing, have high fusion efficiency, and exhibit enzyme deficiencies that render them incapable of growing in certain selective media which support the growth of only the desired fused cells (hybridomas). For example, where the immunized animal is a mouse, one may use P3-X63/Ag8, P3-X63-Ag8.653, NS1/1.Ag 41, Sp210-Ag14, FO, NSO/U, MPC-I1, MPC11-X45-GTG 1.7 and S194/5XX0 BuI; for rats, one may use R210.RCY3, Y3-Ag 1.2.3, IR983F and 4B210; and U-266, GM1500-GRG2, LICR-LON-HMy2 and UC729-6 all may be useful in connection with cell fusions.

In addition to the production of monoclonal antibodies, techniques developed for the production of “chimeric antibodies”, the splicing of mouse antibody genes to human antibody genes to obtain a molecule with appropriate antigen specificity and biological activity, can be used (Morrison et al, Proc Natl Acad Sd 81: 6851-6855, 1984; Neuberger et al, Nature 312: 604-608, 1984; Takeda et al,

Nature 314: 452-454; 1985). Alternatively, techniques described for the production of single-chain antibodies (U.S. Pat. No. 4,946,778) can be adapted to produce influenza-specific single chain antibodies.

Antibody fragments that contain the idiotype of the molecule may be generated by known techniques. For example, such fragments include, but are not limited to, the F(ab′)2 fragment which may be produced by pepsin digestion of the antibody molecule; the Fab′ fragments which may be generated by reducing the disulfide bridges of the F(ab′)2 fragment, and the two Fab fragments which may be generated by treating the antibody molecule with papain and a reducing agent.

Non-human antibodies may be humanized by any methods known in the art. A preferred “humanized antibody” has a human constant region, while the variable region, or at least a complementarity determining region (CDR), of the antibody is derived from a non-human species. The human light chain constant region may be from either a kappa or lambda light chain, while the human heavy chain constant region may be from either an IgM, an IgG (IgG1, IgG2, IgG3, or IgG4) an IgD, an IgA, or an IgE immunoglobulin.

Methods for humanizing non-human antibodies are well known in the art (see U.S. Pat. Nos. 5,585,089, and 5,693,762). Generally, a humanized antibody has one or more amino acid residues introduced into its framework region from a source which is non-human. Humanization can be performed, for example, using methods described in Jones et al. {Nature 321: 522-525, 1986), Riechmann et al, {Nature, 332: 323-327, 1988) and Verhoeyen et al. Science 239:1534-1536, 1988), by substituting at least a portion of a rodent complementarity-determining region (CDRs) for the corresponding regions of a human antibody. Numerous techniques for preparing engineered antibodies are described, e.g., in Owens and Young, J. Immunol. Meth., 168:149-165, 1994. Further changes can then be introduced into the antibody framework to modulate affinity or immunogenicity.

Likewise, using techniques known in the art to isolate CDRs, compositions comprising CDRs are generated. Complementarity determining regions are characterized by six polypeptide loops, three loops for each of the heavy or light chain variable regions. The amino acid position in a CDR and framework region is set out by Kabat et al., “Sequences of Proteins of Immunological Interest,” U.S. Department of Health and Human Services, (1983), which is incorporated herein by reference. For example, hypervariable regions of human antibodies are roughly defined to be found at residues 28 to 35, from residues 49-59 and from residues 92-103 of the heavy and light chain variable regions (Janeway and Travers, Immunobiology, 2nd Edition, Garland Publishing, New York, 1996). The CDR regions in any given antibody may be found within several amino acids of these approximated residues set forth above. An immunoglobulin variable region also consists of “framework” regions surrounding the CDRs. The sequences of the framework regions of different light or heavy chains are highly conserved within a species, and are also conserved between human and murine sequences.

Compositions comprising one, two, and/or three CDRs of a heavy chain variable region or a light chain variable region of a monoclonal antibody are generated. Polypeptide compositions comprising one, two, three, four, five and/or six complementarity determining regions of a monoclonal antibody secreted by a hybridoma are also contemplated. Using the conserved framework sequences surrounding the CDRs, PCR primers complementary to these consensus sequences are generated to amplify a CDR sequence located between the primer regions. Techniques for cloning and expressing nucleotide and polypeptide sequences are well-established in the art [see e.g., Sambrook et al., Molecular Cloning: A Laboratory Manual, 2nd Edition, Cold Spring Harbor, N.Y. (1989)]. The amplified CDR sequences are ligated into an appropriate plasmid. The plasmid comprising one, two, three, four, five and/or six cloned CDRs optionally contains additional polypeptide encoding regions linked to the CDR.

Preferably, the antibody is any antibody specific for a glycan structure of Formula (I) or a fragment thereof. The antibody used in the present invention encompasses any antibody or fragment thereof, either native or recombinant, synthetic or naturally-derived, monoclonal or polyclonal which retains sufficient specificity to bind specifically to the glycan structure according to Formula (I) which is indicative of stem cells. As used herein, the terms “antibody” or “antibodies” include the entire antibody and antibody fragments containing functional portions thereof. The term “antibody” includes any monospecific or bispecific compound comprised of a sufficient portion of the light chain variable region and/or the heavy chain variable region to effect binding to the epitope to which the whole antibody has binding specificity. The fragments can include the variable region of at least one heavy or light chain immunoglobulin polypeptide, and include, but are not limited to, Fab fragments, F(ab′).sub.2 fragments, and Fv fragments.

The antibodies can be conjugated to other suitable molecules and compounds including, but not limited to, enzymes, magnetic beads, colloidal magnetic beads, haptens, fluorochromes, metal compounds, radioactive compounds, chromatography resins, solid supports or drugs. The enzymes that can be conjugated to the antibodies include, but are not limited to, alkaline phosphatase, peroxidase, urease and beta.-galactosidase. The fluorochromes that can be conjugated to the antibodies include, but are not limited to, fluorescein isothiocyanate, tetramethylrhodamine isothiocyanate, phycoerythrin, allophycocyanins and Texas Red. For additional fluorochromes that can be conjugated to antibodies see Haugland, R. P. Molecular Probes: Handbook of Fluorescent Probes and Research Chemicals (1992-1994). The metal compounds that can be conjugated to the antibodies include, but are not limited to, ferritin, colloidal gold, and particularly, colloidal superparamagnetic beads. The haptens that can be conjugated to the antibodies include, but are not limited to, biotin, digoxigenin, oxazalone, and nitrophenol. The radioactive compounds that can be conjugated or incorporated into the antibodies are known to the art, and include but are not limited to technetium 99m, sup.125 I and amino acids comprising any radionuclides, including, but not limited to .sup.14 C, .sup.3 H and .sup.35 S.

Antibodies to glycan structure(s) of Formula (I) may be obtained from any source. They may be commercially available. Effectively, any means which detects the presence of glycan structure(s) on the stem cells is with the scope of the present invention. An example of such an antibody is a H type 1 (clone 17-206; GF 287) antibody from Abeam.

Preferred N-Glycan Structure Types

The invention revealed N-glycans with common core structure of N-glycans, which change according to differentiation and/or individual specific differences.

The N-glycans of embryonic stem cells comprise core structure comprising Manβ4GlcNAc structure in the core structure of N-linked glycan according to the

[Manα3]ₙ₁(Manα6)ₙ₂Manβ4GlcNAcβ4(Fucα6)ₙ₃GlcNAcxR, Formula CGN:
wherein n1, n2 and n3 are integers 0 or 1, independently indicating the presence or absence of the residues, and
wherein the non-reducing end terminal Manα3/Manα6-residues can be elongated to the complex type, especially biantennary structures or to mannose type (high-Man and/or low Man) or to hybrid type structures (for the analysis of the status of stem cells and/or manipulation of the stem cells), wherein xR indicates reducing end structure of N-glycan linked to protein or peptide such as βAsn or βAsn-peptide or βAsn-protein, or free reducing end of N-glycan or chemical derivative of the reducing end produced for analysis.

The preferred Mannose type glycans are according to the formula:

[Mα2]ₙ₁[Mα3]ₙ₂{[Mα2]ₙ₃[Mα6]ₙ₄}[Mα6]ₙ₅{[Mα2]ₙ₆[Mα2]ₙ₇[Mα3]ₙ₈}Mβ4GNβ4[{Fucα6}]ₘGNyR₂ Formula M2:

wherein n1, n2, n3, n4, n5, n6, n7, n8, and m are either independently 0 or 1; with the provision that when n2 is 0, also n1 is 0; when n4 is 0, also n3 is 0; when n5 is 0, also n1, n2, n3, and n4 are 0; when n7 is 0, also n6 is 0; when n8 is 0, also n6 and n7 are 0;

y is anomeric linkage structure αand/or β or linkage from derivatized anomeric carbon, and

R₂ is reducing end hydroxyl, chemical reducing end derivative or natural asparagine N-glycoside derivative such as asparagine N-glycosides including asparagines N-glycoside amino acid and/or peptides derived from protein;

[ ] indicates determinant either being present or absent depending on the value of n1, n2, n3, n4, n5, n6, n7, n8, and m; and

{ } indicates a branch in the structure;

M is D-Man, GN is N-acetyl-D-glucosamine and Fuc is L-Fucose,

and the structure is optionally a high mannose structure, which is further substituted by glucose residue or residues linked to mannose residue indicated by n6.

Several preferred low Man glycans described above can be presented in a single Formula:

[Mα3]ₙ₂[Mα6)]ₙ₄}[Mα6]ₙ₅{[Mα3]ₙ₈}Mβ4GNβ4[{FUCα6}]ₘGNyR₂

wherein n2, n4, n5, n8, and m are either independently 0 or 1; with the provision that when n5 is 0, also n2, and n4 are 0; the sum of n2, n4, n5, and n8 is less than or equal to (m+3); [ ] indicates determinant either being present or absent depending on the value of n2, n4, n5, n8, and m; and { } indicates a branch in the structure;

y and R2 are as indicated above.

Preferred non-fucosylated low-mannose glycans are according to the formula:

[Mα3]ₙ₂([Mα6)]ₙ₄)[Mα6]ₙ₅{[Mα3]ₙ₈}Mβ4GNβ4GNyR₂

wherein n2, n4, n5, n8, and m are either independently 0 or 1,

with the provision that when n5 is 0, also n2 and n4 are 0, and preferably either n2 or n4 is 0,

[ ] indicates determinant either being present or absent depending on the value of, n2, n4, n5, n8,

{ } and ( ) indicates a branch in the structure,

y and R2 are as indicated above.

Preferred Individual Structures of Non-Fucosylated Low-Mannose Glycans

Special Small Structures

Small non-fucosylated low-mannose structures are especially unusual among known N-linked glycans and characteristic glycan group useful for separation of cells according to the present invention. These include:

Mβ4GNβ4GNyR₂

Mα6Mβ4GNβ4GNyR₂

Mα3Mβ4GNβ4GNyR₂ and

Mα6{Mα3}Mβ4GNβ4GNyR₂.

Mβ4GNβ4GNyR₂ trisaccharide epitope is a preferred common structure alone and together with its mono-mannose derivatives Mα6Mβ4GNβ4GNyR₂ and/or Mαα3Mβ4GNβ4GNyR₂, because these are characteristic structures commonly present in glycomes according to the invention. The invention is specifically directed to the glycomes comprising one or several of the small non-fucosylated low-mannose structures. The tetrasaccharides are in a specific embodiment preferred for specific recognition directed to α-linked, preferably α3/6-linked Mannoses as preferred terminal recognition element.

Special Large Structures

The invention further revealed large non-fucosylated low-mannose structures that are unusual among known N-linked glycans and have special characteristic expression features among the preferred cells according to the invention. The preferred large structures include

[Mα3ₙ₂([Mα6]ₙ₄)Mα6{Mα3}Mβ4GNβ4GNyR₂

more specifically

Mα6Mα6{Mα3}Mβ4GNβ4GNyR₂

Mα3Mα6{Mα3}Mβ4GNβ4GNyR₂ and

Mα3(Mα6)Mα6{Mα3}Mβ4GNβ4GNyR₂.

The hexasaccharide epitopes are preferred in a specific embodiment as rare and characteristic structures in preferred cell types and as structures with preferred terminal epitopes. The heptasaccharide is also preferred as a structure comprising a preferred unusual terminal epitope Mα3(Mα6)Mα useful for analysis of cells according to the invention.

Preferred fucosylated low-mannose glycans are derived according to the formula:

[Mα3]ₙ₂{[Mα6]ₙ₄}[Mα6]ₙ₅{[Mα3]ₙ₈}Mβ4GNβ4(Fucα6)GNyR₂

wherein n2, n4, n5, n8, and m are either independently 0 or 1, with the provision that when n5 is 0, also n2 and n4 are 0,

[ ] indicates determinant either being present or absent depending on the value of n2, n4, n5, n8, and m;

{ } and ( ) indicate a branch in the structure.

Preferred Individual Structures of Fucosylated Low-Mannose Glycans

Small fucosylated low-mannose structures are especially unusual among known N-linked glycans and form a characteristic glycan group useful for separation of cells according to the present invention. These include:

Mβ4GNβ4(Fucα6)GNyR₂

Mα6Mβ4GNβ4(Fucα6)GNyR₂

Mα3Mβ4GNβ4(Fucα6)GNyR₂ and

Mα6{Mα3}Mβ4GN β4(Fucα6)GNyR₂.

Mβ4GNβ4(Fucα6)GNyR₂ tetrasaccharide epitope is a preferred common structure alone and together with its monomannose derivatives Mα6Mβ4GNβ4(Fucα6)GNyR₂ and/or Mα3Mβ4GNβ4(Fucα6)GNyR₂, because these are commonly present characteristic structures in glycomes according to the invention. The invention is specifically directed to the glycomes comprising one or several of the small fucosylated low-mannose structures. The tetrasaccharides are in a specific embodiment preferred for specific recognition directed to α-linked, preferably α3/6-linked Mannoses as preferred terminal recognition element.

Special Large Structures

The invention further revealed large fucosylated low-mannose structures that are unusual among known N-linked glycans and have special characteristic expression features among the preferred cells according to the invention. The preferred large structures include

[Mα3]₂([Mα6]ₙ₄)Mα6{Mα3}Mβ4GNβ4(Fucα6)GNyR₂

more specifically

Mα6Mα6{Mα3}Mβ4GNβ4(Fucα6)GNyR₂

Mα3Mα6{Mα3}Mβ4GNβ4(Fucα6)GNyR₂ and

Mα3(Mα6)Mα6{Mα3}Mβ4GNβ4(Fucα6)GNyR₂.

The heptasaccharide epitopes are preferred in a specific embodiment as rare and characteristic structures in preferred cell types and as structures with preferred terminal epitopes. The octasaccharide is also preferred as structure comprising a preferred unusual terminal epitope Mα3(Mα6)Mα useful for analysis of cells according to the invention.

Preferred Non-Reducing End Terminal Mannose-Epitopes

The inventors revealed that mannose-structures can be labeled and/or otherwise specifically recognized on cell surfaces or cell derived fractions/materials of specific cell types. The present invention is directed to the recognition of specific mannose epitopes on cell surfaces by reagents binding to specific mannose structures on cell surfaces.

The preferred reagents for recognition of any structures according to the invention include specific antibodies and other carbohydrate recognizing binding molecules. It is known that antibodies can be produced for the specific structures by various immunization and/or library technologies such as phage display methods representing variable domains of antibodies. Similarly with antibody library technologies, including aptamer technologies and including phage display for peptides, exist for synthesis of library molecules such as polyamide molecules including peptides, especially cyclic peptides, or nucleotide type molecules such as aptamer molecules.

The invention is specifically directed to specific recognition of high-mannose and low-mannose structures according to the invention. The invention is specifically directed to recognition of non-reducing end terminal Manα-epitopes, preferably at least disaccharide epitopes, according to the formula:

[Mα2]ₘ₁[Mαx]ₘ₂[Mα6]ₘ₃{{[Mα2]ₘ₉[Mα2]ₘ₈[Mα3]ₘ₇}ₘ₁₀(Mβ4[GN]ₘ₄)ₘ₅}ₘ₆yR₂

wherein m1, m2, m3, m4, m5, m6, m7, m8, m9 and m10 are independently either 0 or 1; with the provision that when m3 is 0, then m1 is 0, and when m7 is 0 then either m1-5 are 0 and m8 and m9 are 1 forming a Mα2Mα2-disaccharide, or both m8 and m9 are 0;

y is anomeric linkage structure α and/or β or linkage from derivatized anomeric carbon, and

R₂ is reducing end hydroxyl or chemical reducing end derivative

and x is linkage position 3 or 6 or both 3 and 6 forming branched structure,

{ } indicates a branch in the structure.

The invention is further directed to terminal Mα2-containing glycans containing at least one Mα2-group and preferably Mα2-group on each branch so that m1 and at least one of m8 or m9 is 1. The invention is further directed to terminal Mα3 and/or Mα6-epitopes without terminal Mα2-groups, when all m1, m8 and m9 are 1.

The invention is further directed in a preferred embodiment to the terminal epitopes linked to a M-residue and for application directed to larger epitopes. The invention is especially directed to Mβ4GN-comprising reducing end terminal epitopes.

The preferred terminal epitopes comprise typically 2-5 monosaccharide residues in a linear chain. According to the invention short epitopes comprising at least 2 monosaccharide residues can be recognized under suitable background conditions and the invention is specifically directed to epitopes comprising 2 to 4 monosaccharide units and more preferably 2-3 monosaccharide units, even more preferred epitopes include linear disaccharide units and/or branched trisaccharide non-reducing residue with natural anomeric linkage structures at reducing end. The shorter epitopes may be preferred for specific applications due to practical reasons including effective production of control molecules for potential binding reagents aimed for recognition of the structures.

The shorter epitopes such as Mα2M is often more abundant on target cell surface as it is present on multiple arms of several common structures according to the invention.

Preferred Disaccharide Epitopes Include

Manα2Man, Manα3Man, Manα6Man, and more preferred anomeric forms Manα2Manα, Manα3Manβ, Manα6Manβ, Manα3Manα and Manα6Manα.

Preferred branched trisaccharides include Manα3(Manα6)Man, Manα3(Manα6)Manβ, and Manα3(Manα6)Manα.

The invention is specifically directed to the specific recognition of non-reducing terminal Manα2-structures especially in context of high-mannose structures.

The invention is specifically directed to following linear terminal mannose epitopes:

a) preferred terminal Manα2-epitopes including following oligosaccharide sequences:

Manα2Man,

Manα2Manα,

Manα2Manα2Man, Manα2Manα3Man, Manα2Manα6Man,

Manα2Manα2Manα, Manα2Manα3Manβ, Manα2Manα6Manα,

Manα2Manα2Manα3Man, Manα2Manα3Manα6Man, Manα2Manα6Manα6Man

Manα2Manα2Manα3Manβ, Manα2Manα3Manα6Manβ, Manα2Manα6Manα6Manβ;

The invention is further directed to recognition of and methods directed to non-reducing end terminal Manα3- and/or Manα6-comprising target structures, which are characteristic features of specifically important low-mannose glycans according to the invention. The preferred structural groups include linear epitopes according to b) and branched epitopes according to the c3) especially depending on the status of the target material.

b) preferred terminal Manα3- and/or Manα6-epitopes including following oligosaccharide sequences:

Manα3Man, Manα6Man, Manα3Manβ, Manα6Manβ, Manα3Manα, Manα6Manα,

Manα3Manα6Man, Manα6Manα6Man, Manα3Manα6Manβ, Manα6Manα6Manβ

and to following:

c) branched terminal mannose epitopes are preferred as characteristic structures of especially high-mannose structures (c1 and c2) and low-mannose structures (c3), the preferred branched epitopes including:

c1) branched terminal Manα2-epitopes

Manα2Manα3(Manα2Manα6)Man, Manα2Manα3(Manα2Manα6)Manα,

Manα2Manα3(Manα2Manα6)Manα6Man, Manα2Manα3(Manα2Manα6)Manα6Manβ,

Manα2Manα3(Manα2Manα6)Manα6(Manα2Manα3)Man,

Manα2Manα3(Manα2Manα6)Manα6(Manα2Manα2Manα3)Man,

Manα2Manα3(Manα2Manα6)Manα6(Manα2Manα3)Manβ

Manα2Manα3(Manα2Manα6)Manα6(ManαManα2Manα3)Manβ

c2) branched terminal Manα2- and Manα3 or Manα6-epitopes

according to formula when m1 and/or m8 and/m9 is 1 and the molecule comprise at least one nonreducing end terminal Manα3 or Manα6-epitope

c3) branched terminal Manα3 or Manα6-epitopes

Manα3(Manα6)Man, Manα3(Manα6)Manβ, Manα3(Manα6)Manα,

Manα3(Manα6)Manα6Man, Manα3(Manα6)Manα6Manβ,

Manα3(Manα6)Manα6(Manα3)Man, Manα3(Manα6)Manα6(Manα3)Manβ

The present invention is further directed to increase the selectivity and sensitivity in recognition of target glycans by combining recognition methods for terminal Manα2 and Manα3 and/or Manα6-comprising structures. Such methods would be especially useful in context of cell material according to the invention comprising both high-mannose and low-mannose glycans.

Complex Type N-Glycans

According to the present invention, complex-type structures are preferentially identified by mass spectrometry, preferentially based on characteristic monosaccharide compositions, wherein HexNAc≧4 and Hex≧3. In a more preferred embodiment of the present invention, 4≦HexNAc≦20 and 3≦Hex≦21, and in an even more preferred embodiment of the present invention, 4≦HexNAc≦10 and 3≦Hex≦11. The complex-type structures are further preferentially identified by sensitivity to endoglycosidase digestion, preferentially N-glycosidase F detachment from glycoproteins. The complex-type structures are further preferentially identified in NMR spectroscopy based on characteristic resonances of the Manα3(Manα6)Manβ4GlcNAcβ4GlcNAc N-glycan core structure and GlcNAc residues attached to the Manα3 and/or Manα6 residues.

Beside Mannose-type glycans the preferred N-linked glycomes include GlcNAcβ2-type glycans including Complex type glycans comprising only GlcNAcβ2-branches and Hydrid type glycan comprising both Mannose-type branch and GlcNAcβ2-branch.

GlcNAcβ2-Type Glycans

The invention revealed GlcNAcβ2Man structures in the glycomes according to the invention.

Preferably GlcNAcβ2Man-structures comprise one or several of GlcNAcβ2Manα-structures, more preferably GlcNAcβ2Manα3- or GlcNAcβ2Manα6-structure.

The Complex type glycans of the invention comprise preferably two GlcNAcβ2Manα structures, which are preferably GlcNAcβ2Manα3 and GlcNAcβ2Manα6. The Hybrid type glycans comprise preferably GlcNAcβ2Manα3-structure.

The present invention is directed to at least one of natural oligosaccharide sequence structures and structures truncated from the reducing end of the N-glycan according to

the Formula CO1 (also referred as GNβ2):

[R₁GNβ2]ₙ₁[Mα3]ₙ₂{[R₃]ₙ₃[GNβ2]ₙ₄Mα6}ₙ₅Mβ4GNXyR₂,

with optionally one or two or three additional branches according to formula [RₓGNβz]ₙₓ linked to Mα6-, Mα3-, or Mβ4, and Rₓ may be different in each branch

wherein n1, n2, n3, n4, n5 and nx, are either 0 or 1, independently,

with the provision that when n2 is 0 then n1 is 0 and when n3 is 1 and/or n4 is 1 then n5 is also 1,

and at least n1 or n4 is 1, or n3 is 1;

when n4 is 0 and n3 is 1 then R₃ is a mannose type substituent or nothing and

wherein X is a glycosidically linked disaccharide epitope β4(Fucα6)ₙGN, wherein n is 0 or 1, or X is nothing and

y is anomeric linkage structure α and/or β or linkage from derivatized anomeric carbon, and

R₁, Rₓ and R₃ indicate independently one, two or three natural substituents linked to the core structure,

R₂ is reducing end hydroxyl, chemical reducing end derivative or natural asparagine N-glycoside derivative such as asparagine N-glycosides including asparagines N-glycoside amino acids and/or peptides derived from protein; [ ] indicate groups either present or absent in a linear sequence, and { } indicates branching which may be also present or absent.

Elongation of GlcNAcβ2-Type Structures Forming Complex/Hydrid Type Structures

The substituents R₁, Rₓ and R₃ may form elongated structures. In the elongated structures R₁, and Rₓ represent substituents of GlcNAc (GN) and R₃ is either substituent of GlcNAc or when n4 is 0 and n3 is 1 then R3 is a mannose type substituent linked to Manα6-branch forming a Hybrid type structure. The substituents of GN are monosaccharide Gal, GalNAc, or Fuc and/or acidic residue such as sialic acid or sulfate or phosphate ester.

GlcNAc or GN may be elongated to N-acetyllactosaminyl also marked as GalβGN or di-N-acetyllactosdiaminyl GalNAcβGlcNAc, preferably GalNAcβ4GlcNAc. LNβ2M can be further elongated and/or branched with one or several other monosaccharide residues such as galactose, fucose, SA or LN-unit(s) which may be further substituted by SAα-structures,

and/or Mα6 residue and/or Mα3 residue can be further substituted by one or two β6-, and/or β4-linked additional branches according to the formula;

and/or either of Mα6 residue or Mα3 residue may be absent;

and/or Mα6-residue can be additionally substituted by other Manα units to form a hybrid type structures;

and/or Manβ4 can be further substituted by GNβ4,

and/or SA may include natural substituents of sialic acid and/or it may be substituted by other SA-residues preferably by α8- or α9-linkages.

The SAα-groups are linked to either 3- or 6-position of neighboring Gal residue or on 6-position of GlcNAc, preferably 3- or 6-position of neighboring Gal residue. In separately preferred embodiments the invention is directed to structures comprising solely 3-linked SA or 6-linked SA, or mixtures thereof.

Preferred Complex Type Structures

Incomplete Monoantennary N-Glycans

The present invention revealed incomplete Complex monoantennary N-glycans, which are unusual and useful for characterization of glycomes according to the invention. The most of the incomplete monoantennary structures indicate potential degradation of biantennary N-glycan structures and are thus preferred as indicators of cellular status. The incomplete Complex type monoantennary glycans comprise only one GNβ2-structure.

The invention is specifically directed to structures according to the Formula CO1 or Formula GNb2 above when only n1 is 1 or n4 is 1 and mixtures of such structures.

The preferred mixtures comprise at least one monoantennary complex type glycans

A) with a single branch likely from a degradative biosynthetic process:

R₁GNβ2Mα3β4GNXyR₂

R₃GNβ2Mα6Mβ4GNXyR₂ and

B) with two branches comprising mannose branches

R₁GNβ2Mα3{Mα6}ₙ₅Mβ4GNXyR₂ B1)

Mα3{R₃GNβ2Mα6}ₙ₅Mβ4GNXyR₂ B2)

The structure B2 is preferred over A structures as product of degradative biosynthesis, it is especially preferred in context of lower degradation of Manα3-structures. The structure B1 is useful for indication of either degradative biosynthesis or delay of biosynthetic process.

Biantennary and Multiantennary Structures

The inventors revealed a major group of biantennary and multiantennary N-glycans from cells according to the invention. The preferred biantennary and multiantennary structures comprise two GNβ2 structures. These are preferred as an additional characteristic group of glycomes according to the invention and are represented according to the Formula CO2:

R₁GNβ2Mα3{R3GNβMα6}Mβ4GNXyR₂

with optionally one or two or three additional branches according to formula [RₓGNβz]ₙₓ linked to Mα6-, Mα3-, or Mβ4 and Rₓ may be different in each branch

wherein nx is either 0 or 1,

and other variables are according to the Formula CO1.

Preferred Biantennary Structure

A biantennary structure comprising two terminal GNβ-epitopes is preferred as a potential indicator of degradative biosynthesis and/or delay of biosynthetic process. The more preferred structures are according to the Formula CO2 when R₁ and R₃ are nothing.

Elongated Structures

The invention revealed specific elongated complex type glycans comprising Gal and/or GalNAc-structures and elongated variants thereof. Such structures are especially preferred as informative structures because the terminal epitopes include multiple informative modifications of lactosamine type, which characterize cell types according to the invention.

The present invention is directed to at least one of natural oligosaccharide sequence structure or group of structures and corresponding structure(s) truncated from the reducing end of the N-glycan according to the Formula CO3:

[R₁Gal[NAc]ₒ₂βz2]ₒ₁GNβ2Mα3{[R₁Gal[ANc]ₒ₄βz2]ₒ₃GNβ2Mα6}Mβ4GNXyR₂,

with optionally one or two or three additional branches according to formula [RₓGNβz1]ₙₓ linked to Mα6-, Mα3-, or Mβ4 and Rₓ may be different in each branch

wherein nx, o1, o2, o3, and o4 are either 0 or 1, independently,

with the provision that at least o1 or o3 is 1, in a preferred embodiment both are 1;

z2 is linkage position to GN being 3 or 4, in a preferred embodiment 4;

z1 is linkage position of the additional branches;

R₁, Rₓ and R₃ indicate one or two a N-acetyllactosamine type elongation groups or nothing,

{ } and ( ) indicates branching which may be also present or absent,

other variables are as described in Formula GNb2.

Galactosylated Structures

The inventors characterized useful structures especially directed to digalactosylated structure

GalβzGNβ2Mα3{GalβzGNβ2Mα6}Mβ4GNXyR₂,

and monogalactosylated structures:

GalβzGNβ2Mα3{GNβ2Mα6}Mβ4GNXyR₂,

GNβ2Mα3{GalβzGNβ2Mα6}Mβ4GNXyR₂,

and/or elongated variants thereof preferred for carrying additional characteristic terminal structures useful for characterization of glycan materials

R₁GalβzGNβ2Mα3{R₃GalβzGNβ2Mα6}Mβ4GNXyR₂

R₁GalβzGNβ2Mα3{GNβ2Mα6}Mβ4GNXyR₂, and

GNβ2Mα3{R₃GalβzGNβ2Mα6}Mβ4GNXyR₂.

Preferred elongated materials include structures wherein R₁ is a sialic acid, more preferably NeuNAc or NeuGc.

LacdiNAc-Structure Comprising N-Glycans

The present invention revealed for the first time LacdiNAc, GalNAcβGlcNAc structures from the cell according to the invention. Preferred N-glycan lacdiNAc structures are included in structures according to the Formula CO1, when at least one the variable o2 and o4 is 1.

The Major Acidic Glycan Types

The acidic glycomes mean glycomes comprising at least one acidic monosaccharide residue such as sialic acids (especially NeuNAc and NeuGc) forming sialylated glycome, HexA (especially GlcA, glucuronic acid) and/or acid modification groups such as phosphate and/or sulphate esters.

According to the present invention, presence of sulphate and/or phosphate ester (SP) groups in acidic glycan structures is preferentially indicated by characteristic monosaccharide compositions containing one or more SP groups. The preferred compositions containing SP groups include those formed by adding one or more SP groups into non-SP group containing glycan compositions, while the most preferential compositions containing SP groups according to the present invention are selected from the compositions described in the acidic N-glycan fraction glycan group Tables of the present invention. The presence of phosphate and/or sulphate ester groups in acidic glycan structures is preferentially further indicated by the characteristic fragments observed in fragmentation mass spectrometry corresponding to loss of one or more SP groups, the insensitivity of the glycans carrying SP groups to sialidase digestion. The presence of phosphate and/or sulphate ester groups in acidic glycan structures is preferentially also indicated in positive ion mode mass spectrometry by the tendency of such glycans to form salts such as sodium salts as described in the Examples of the present invention. Sulphate and phosphate ester groups are further preferentially identified based on their sensitivity to specific sulphatase and phosphatase enzyme treatments, respectively, and/or specific complexes they form with cationic probes in analytical techniques such as mass spectrometry.

Sialylated Complex N-Glycan Glycomes

The present invention is directed to at least one of natural oligosaccharide sequence structures and structures truncated from the reducing end of the N-glycan according to the Formula

[{SAα3/6}ₛ₁LNβ2]ᵣ₁Mα3{({SAα3/6}ₛ₂LNβ2)ᵣ₂Mα6}ᵣ₈{M[β4GN[β4{Fucα6}ᵣ₃GN]ᵣ₄]ᵣ₅}ᵣ₆ (I)

with optionally one or two or three additional branches according to formula

{SAα3/6}ₛ₃LNβ, (IIb)

wherein r1, r2, r3, r4, r5, r6, r7 and r8 are either 0 or 1, independently,

wherein s1, s2 and s3 are either 0 or 1, independently,

with the provision that at least r1 is 1 or r2 is 1, and at least one of s1, s2 or s3 is 1.

LN is N-acetyllactosaminyl also marked as GalβGN or di-N-acetyllactosdiaminyl

GalNAcβGlcNAc preferably GalNAcβ4GlcNAc, GN is GlcNAc, M is mannosyl-,

with the provision that LNβ2M or GNβ2M can be further elongated and/or branched with one or several other monosaccharide residues such as galactose, fucose, SA or LN-unit(s) which may be further substituted by SAα-structures,

and/or one LNβ can be truncated to GNβ

and/or Mα6 residue and/or Mα3 residue can be further substituted by one or two β6-, and/or β4-linked additional branches according to the formula,

and/or either of Mα6 residue or Mα3 residue may be absent;

and/or Mα6-residue can be additionally substituted by other Manα units to form a hybrid type structures

and/or Manβ4 can be further substituted by GNβ4,

and/or SA may include natural substituents of sialic acid and/or it may be substituted by other SA-residues preferably by α8- or α9-linkages.

( ), { }, └ ┘ and [ ] indicate groups either present or absent in a linear sequence. { }indicates branching which may be also present or absent.

The SAα-groups are linked to either 3- or 6-position of neighboring Gal residue or on 6-position of GlcNAc, preferably 3- or 6-position of neighboring Gal residue. In separately preferred embodiments the invention is directed structures comprising solely 3-linked SA or 6-linked SA, or mixtures thereof. In a preferred embodiment the invention is directed to glycans wherein r6 is 1 and r5 is 0, corresponding to N-glycans lacking the reducing end GlcNAc structure.

The LN unit with its various substituents can be represented in a preferred general embodiment by the formula:

[Gal(NAc)ₙ₁α3]ₙ₂{Fucα2}ₙ₃Gal(NAc)ₙ₄β3/4{Fucα4/3}ₙ₅GlcNAcβ

wherein n1, n2, n3, n4, and n5 are independently either 1 or 0,

with the provision that the substituents defined by n2 and n3 are alternative to the presence of SA at the non-reducing end terminal structure;

the reducing end GlcNAc-unit can be further β3- and/or β6-linked to another similar LN-structure forming a poly-N-acetyllactosamine structure with the provision that for this LN-unit n2, n3 and n4 are 0,

the Gal(NAc)β and GlcNAcβ units can be ester linked a sulphate ester group;

( ) and [ ] indicate groups either present or absent in a linear sequence; { } indicates branching which may be also present or absent.

LN unit is preferably Galβ4GN and/or Galβ3GN. The inventors revealed that hESCs can express both types of N-acetyllactosamine, and therefore the invention is especially directed to mixtures of both structures. Furthermore, the invention is directed to special relatively rare type 1 N-acetyllactosamines, Galβ3GN, without any non-reducing end/site modification, also called lewis c-structures, and substituted derivatives thereof, as novel markers of hESCs.

Hybrid Type Structures

According to the present invention, hybrid-type or monoantennary structures are preferentially identified by mass spectrometry, preferentially based on characteristic monosaccharide compositions, wherein HexNAc=3 and Hex≧2. In a more preferred embodiment of the present invention 2≦Hex≦11, and in an even more preferred embodiment of the present invention 2≦Hex≦9. The hybrid-type structures are further preferentially identified by sensitivity to exoglycosidase digestion, preferentially α-mannosidase digestion when the structures contain non-reducing terminal α-mannose residues and Hex≧3, or even more preferably when Hex≧4, and to endoglycosidase digestion, preferentially N-glycosidase F detachment from glycoproteins. The hybrid-type structures are further preferentially identified in NMR spectroscopy based on characteristic resonances of the Manα3(Manα6)Manβ4GlcNAcβ4GlcNAc N-glycan core structure, a GlcNAcβ residue attached to a Manα residue in the N-glycan core, and the presence of characteristic resonances of non-reducing terminal α-mannose residue or residues.

The monoantennary structures are further preferentially identified by insensitivity to α-mannosidase digestion and by sensitivity to endoglycosidase digestion, preferentially N-glycosidase F detachment from glycoproteins. The monoantennary structures are further preferentially identified in NMR spectroscopy based on characteristic resonances of the Manα3Manβ4GlcNAcβ4GlcNAc N-glycan core structure, a GlcNAcβ residue attached to a Manα residue in the N-glycan core, and the absence of characteristic resonances of further non-reducing terminal α-mannose residues apart from those arising from a terminal α-mannose residue present in a ManαManβ sequence of the N-glycan core.

The invention is further directed to the N-glycans when these comprise hybrid type structures according to the Formula HY1:

R₁GNβ2Mα3{[R₃]ₙ₃Mα6}Mβ4GNXyR₂,

wherein n3, is either 0 or 1, independently,

and wherein X is glycosidically linked disaccharide epitope β4(Fucα6)ₙGN, wherein n is 0 or 1, or

X is nothing and

y is anomeric linkage structure α and/or β or linkage from derivatized anomeric carbon, and

R₁ indicate nothing or substituent or substituents linked to GlcNAc,

R₃ indicates nothing or Mannose-substituent(s) linked to mannose residue, so that each of R₁, and

R₃ may correspond to one, two or three, more preferably one or two, and most preferably at least one natural substituents linked to the core structure,

R₂ is reducing end hydroxyl, chemical reducing end derivative or natural asparagine N-glycoside derivative such as asparagine N-glycosides including asparagines N-glycoside amino acids and/or peptides derived from protein; [ ] indicate groups either present or absent in a linear sequence, and { } indicates branching which may be also present or absent.

Preferred Hybrid Type Structures

The preferred hybrid type structures include one or two additional mannose residues on the preferred core structure.

R₁GNβ2Mα3{[Mα3]ₘ₁([Mα6])ₘ₂Mα6}Mβ4GNXyR₂, Formula HY2

wherein and m1 and m2 are either 0 or 1, independently,

{ } and ( ) indicates branching which may be also present or absent,

other variables are as described in Formula HY1.

Furthermore the invention is directed to structures comprising additional lactosamine type structures on GNβ2-branch. The preferred lactosamine type elongation structures includes N-acetyllactosamines and derivatives, galactose, GalNAc, GlcNAc, sialic acid and fucose.

Preferred structures according to the formula HY2 include:

Structures containing non-reducing end terminal GlcNAc as a specific preferred group of glycans

GNβ2Mα3{Mα3Mα6}Mβ4GNXyR₂,

GNβ2Mα3{Mα6Mα6}Mβ4GNXyR₂,

GNβ2Mα3{Mα3(Mα6)Mα6}Mβ4GNXyR₂,

and/or elongated variants thereof

R₁GNβ2Mα3{Mα3Mα6}Mβ4GNXyR₂,

R₁GNβ2Mα3{Mα6Mα6}Mβ4GNXyR₂,

R₁GNβ2Mα3{Mα3(Mα6)Mα6}Mβ4GNXyR₂,

[R₁Gal[NAc]ₒ₂βz]ₒ₁GNβ2Mα3{[(Mα6)]ₘ₂Mα6}ₙ₅Mβ4GNXyR₂, Formula HY3

wherein n5, m1, m2, o1 and o2 are either 0 or 1, independently,

z is linkage position to GN being 3 or 4, in a preferred embodiment 4,

R₁ indicates one or two a N-acetyllactosamine type elongation groups or nothing,

{ } and ( ) indicates branching which may be also present or absent,

other variables are as described in Formula HY1.

Preferred structures according to the formula HY3 include especially structures containing non-reducing end terminal Galβ, preferably Galβ3/4 forming a terminal N-acetyllactosamine structure. These are preferred as a special group of Hybrid type structures, preferred as a group of specific value in characterization of balance of Complex N-glycan glycome and High mannose glycome: GalβzGNβ2Mα3{Mα3Mα6}Mβ4GNXyR₂, GalβzGNβ2Mα3{Mα6Mα6}Mβ4GNXyR₂, GalβzGNβ2Mα3{Mα3(Mα6)Mα6}Mβ4GNXyR₂,

and/or elongated variants thereof preferred for carrying additional characteristic terminal structures useful for characterization of glycan materials R₁GalβzGNβMα3{Mα3Mα6}Mβ4GNXyR₂, R₁GalβzGNβ2Mα3{Mα6Mα6}Mβ4GNXyR₂, R₁GalβzGNβ2Mα3{Mα3(Mα6)Mα6}Mβ4GNXyR₂. Preferred elongated materials include structures wherein R₁ is a sialic acid, more preferably NeuNAc or NeuGc.

Structures Associated with Nondifferentiated hESC

The Tables 1 and 2 show specific structure groups with specific monosaccharide compositions associated with the differentiation status of human embryonic stem cells.

The Structures Present in Higher Amount in hESCs than in Corresponding Differentiated Cells

The invention revealed novel structures present in higher amounts in hESCs than in corresponding differentiated cells.

The preferred hESC enriched glycan groups are represented by groups hESC-i to hESC-ix, corresponding to several types of N-glycans. The glycans are preferred in the order from hESC-i to hESC-ix, based on the relative specificity for the non-differentiated hESCs, the differences in expression are shown in Tables 1 and 2. The glycans are grouped based on similar composition and similar structures present to group comprising Complex type N-glycans other preferred glycan groups,

Complex Type Glycans

hESC-i, Biantennary-Size Complex-Type N-Glycans

The highest specific expression in hESCs was revealed for a specific group of biantennary complex type N-glycan structures. This group includes neutral glycans including H5N4F1, H5N4F2, H5N4F3; and sialylated glycans G2H5N4, G1H5N4, S1H5N4F2, G1H5N4F1, S1G1H5N4, S1H5N4F3, S2H5N4F1, S1H5N4, and S1H5N4F1.

Preferred Structural Subgroups of the Biantennary Complex Type Glycans Include Neutral Fucosylated Glycans and NeuAc Comprising Fucosylated Glycans and Glycans comprising NeuGc.

Neutral Fucosylated Glycans

The group of neutral glycans forms a homogenous group with typical composition of biantennary N-glycans and one, two or three fucose residues. This group shares a common composition:

H₅N₄F_{q}

Wherein

q is an integer being 1, 2 or 3.

The preferred structures in this group include

[Fucα]ₘGalβGNβ2Manα3([Fucα]ₙGalβGNβ2Manα6)Manβ4GN4(Fucα6)GN,

wherein m and n are 0 or 1, GN is GlcNAc. The structures are preferably core fucosylated, when there is only one fucose. (The core fucosylation was revealed by NMR-analysis of the hESC glycans.) The fucose residues at the antennae (branches) are preferably either Fucα2-structures linked to Gal or Fucα3/4-structures, preferably Fucα3, linked to GlcNAc of the terminal N-acetyllactosamines. Preferred Fucosylated Terminal Epitopes [Fucα]GalβGlcNAcβ2Manα

Preferred Lewis x Epitopes

The preferred terminal epitopes, which can be recognized from hESCs by specific binder molecules, include Lewis x, Galβ4(Fucα3)GlcNAcβ, more preferably Galβ4(Fucα3)GlcNAcβ2Manα, based on binding of specific Lewis x recognizing monoclonal antibody.

The invention is further directed to the recognition of the Lewis x structure as a specific preferred arm of N-glycan selected from the group Galβ4(Fucα3)GlcNAcβ2Manα3Manβ(Lexβ2Manα3-arm) and/or Galβ4(Fucα3)GlcNAcβ2Manα6Manβ (Lexβ2Manα6-arm). The invention is directed to selection and development of reagents for the specific fucosylated N-glycan arms for recognition of N-glycans on the human embryonic stem cells and derivatives.

The H-antigens on N-glycans includes preferably the epitope Fucα2GalβGlcNAcβ, preferably H type I Fucα2Galβ3GlcNAcβ or H type II structure Fucα2Galβ4GlcNAcβ, more preferably Fucα2Galβ4GlcNAcβ, and most preferably Fucα2Galβ4GlcNAcβ2Manα.

The invention is further directed to the recognition of the H type II structure as a specific preferred arm of N-glycan selected from the group

Fucα2Galβ4GlcNAcβ2Manα3Manβ (HLacNAcβ2Manα3-arm) and/or Fucα2Galβ4GlcNAcβ2Manα6Manβ (HLacNAcβ2Manα6-arm). The invention is directed to selection and development of reagents for the specific fucosylated N-glycan arms for recognition of N-glycans on the human embryonic stem cells and derivatives.

Preferred neutral difucosylated structures include glycans comprising core fucose and the terminal Lewis x or H-antigen on either arm of the biantennary N-glycan according to the formulae:

Gaβ4(Fucα3)GNβ2Manα3/6(GalβGNβ2Manα6/3)Manβ4GNβ4(Fucα6)GN, and/or

Fucα2GalβGNβ2Manα3/6(GaβGNβ2Manα6/3)Manβ4GNβ4(Fucα6)GN.

Preferred neutral trifucosylated structures includes glycans comprising core fucose and the terminal Lewis x or H-antigen on either arm of the biantennary N-glycan according to the formulae:

Galβ4(Fucα3)GNβ2Manα3/6([Fucα]GalβGNβ2Manα6/3)Manβ4GNβ4(Fucα6)GN, and/or

Fucα2GalβGNβ2Manα3/6([Fucα]GalβGNβ2Manα6/3)Manβ4GNβ4(Fucα6)GN,

Wherein the molecules comprise two H-structures, Lewis x in one arm and H-structure in the the other arm or two Lewis x structures:

Fucα2GalβGNβ2Manα3(Fucα2GalβGNβ2Manα6)Manβ4GNβ4(Fucα6)GN,

Galβ4(Fucα3)GNβ2Manα3/6(Fucα2GalβGNβ2Manα6/3)Manβ4GNβ4(Fucα6)GN

Galβ4(Fucα3)GNβ2Manα3(Galβ4(Fucα3)GNβ2Manα6)Manβ4GNβ4(Fucα6)GN,

Or molecules comprising Lewis y on one arm:

Fucα2Galβ4(Fucα3)GNβ2Manα3/6(GalβGNβ2Manα6/3)Manβ4GNβ4(Fucα6)GN

NeuAc Comprising Fucosylated Glycans

The sialylated glycans include NeuAc comprising fucosylated glycans with formulae: S1H5N4F2, S1H5N4F3, S2H5N4F1, S1H5N4, and S1H5N4F1. This group shares composition:

SₖH₅N₄F_{q}

Wherein

k is an integer being 1 or 2

q is an integer from 0 to 3.

The group comprises monosialylated glycans with all levels of fucosylation and disialylated glycan with single fucose. The preferred subgroups of this category include low fucosylation level glycans comprising no or one fucose residue (low fucosylation) and glycans with two or three fucose residues.

Preferred Biantennary Structures with Low Fucosylation

The preferred biantennary structures according to the invention include structures according to the Formula:

[NeuAcα]₀₋₁GalβGNβ2Manα3([NeuAcα]₀₋₁GalβGNβ2Manα6)Manβ4GNβ4(Fucα6)₀₋₁GN,

The GalβGlcNAc structures are preferably Galβ4GlcNAc-structures (type II N-acetyllactosamine antennae). The presence of type 2 structures was revealed by specific β4-linkage cleaving galactosidase (D. pneumoniae).

In a preferred embodiment the sialic acid is NeuAcα6- and the glycan comprises the NeuAc linked to Manα3-arm of the molecule. The assignment is based on the presence of α6-linked sialic acid revealed by specific sialidase digestion and the known branch specificity of the α6-sialyltransferase (ST6GalI). NeuAcα6GalβGNβ2Manα3([NeuAcα]₀₋₁GalβGNβ2Manα6)Manβ4GNβ4(Fucα6)₀₋₁GN, more preferably type II structures:

NeuAcα6Galβ4GNβ2manα3([NeuAcα]₀₋₁Galβ4GNβ2Manα6)Manβ4GNβ4(Fucα6)₀₋₁GN.

The invention thus revealed preferred terminal epitopes, NeuAcα6GalβGN, NeuAcα6GalβGNβ2Man, NeuAcα6GalβGNβ2Manα3, to be recognized by specific binder molecules. It is realized that higher specificity preferred for application in context of similar structures can be obtained by using binder recognizing longer epitopes and thus differentiating e.g. between N-glycans and other glycan types in context of the terminal epitopes.

Preferred Difucosylated and Sialylated Structures

Preferred difucosylated sialylated structures include structures, wherein the one fucose is in the core of the N-glycan and

a) one fucose on one arm of the molecule, and sialic acid is on the other arm (antenna of the molecule and the fucose is in Lewis x or H-structure:

Galβ4(Fucα3)GNβ2Manα3/6(NeuNAcαGalβGNβ2Manα6/3)Manβ4GNβ4(Fucα6)GN, and/or

Fucα2GalβGNβ2Manα3/6(NeuNAcαGalβGNβ2Manα6/3)Manβ4GNβ4(Fucα6)GN, and when the sialic acid is α6-linked preferred antennary structures contain preferably the sialyl-lactosamine on α3-linked arm of the molecule according to formula:

Galβ4(Fucα3)GNβ2Manβ6(NeuNAcα6Galβ4GNβ2Manα3)Manβ4GNβ4(Fucα6)GN, and/or

Fucα2GalβGNβ2Manα6(NeuNAcα6Galβ4GNβ2Manα3)Manβ4GNβ4(Fucα6)GN.

It is realized that the structures, wherein the sialic acid and fucose are on different arms of the molecules can be recognized as characteristic specific epitopes.

b) Fucose and NeuAc are on the same arm in a structure:

NeuNAcα3Galβ3/4(Fucα4/3)GNβ2Manα3/6(GalβGNβ2Manα6/3)Manβ4GNβ4(Fuc═6)GN, and more preferably sialylated and fucosylated sialyl-Lewis x structures are preferred as a characteristic and bioactive structures:

NeuNAcα3Galβ4(Fucα3 )GNβ2Manβ3/6(Galβ4GNβ2Manα6/3)Manβ4GNβ4(Fucα6)GN.

Preferred Sialylated Trifucosylated Structures

Preferred sialylated trifucosylated structures include glycans comprising core fucose and the terminal sialyl-Lewis x or sialyl-Lewis a, preferably sialyl-Lewis x due to relatively large presence of type 2 lactosamines, or Lewis y on either arm of the biantennary N-glycan according to the formulae:

NeuNAcα3Galβ4(Fucα3)GNβ2Manα3/6([Fucα]GalβGNβ2Manα6/3)Manβ4GNβ4(Fucα6)GN,

and/or

Fucα2Galβ4(Fucα3)GNβ2Manα3/6(NeuNAcα3/6GalβGNβ2Manα6/3)Manβ4GNβ4(Fucα6)GN.

NeuNAc is preferably α-linked on the same arm as fucose due to known biosynthetic preferance.

When the structure comprises NeuNAcα6, this is preferably linked to form NeuNAcα6Galβ4GlcNAcβ2Manα3-arm of the molecule.

Glycans Comprising N-Lycolylneuraminic Acid

The invention is directed to glycans comprising N-glycolylneuraminic acid with following compositions G2H5N4, G1H5N4, G1H5N4F1, and S1G1H5N4. The compositions form a group of compositions with composition:

GₘSₖH₅N₄F_{q}

wherein

m is an integer being 1 or 2,

k is an integer being 0 or 1, and

q is an integer being 0 or 1.

The invention is further directed to the structures according to the formula:

[NeuXα]₀₋₁GalβGNβ2Manα3/6([NeuXα]₀₋₁GalβGNβ2Manα6/3)Manβ4GNβ4(Fucα6)₀₋₁GN,

wherein X is Gc or Ac, and the sialic acids are linked by α3- and/or α6-linkages.

It is further realized that it is useful to analyze the NeuGc comprising structures in context of contamination by animal protein and or animal derived NeuGc-monosaccharide or glycoconjugate comprising material.

hESC-ii, Complex-Fucosylated N-Glycans

The invention is further directed to following neutral glycans including H5N4F2, H5N4F3, H4N5F3; and sialylated glycans including S1H7N6F2, S1H7N6F3, S1H5N4F2, S1H6N5F2, S1H6N4F2, S1H5N4F3, S1H4N5F2, S2H6N5F2, S1H6N5F3;

preferentially with α1,2-, α1,3-, and/or α1,4-linked fucose residues within the N-acetyllactosamine antenna sequence Galβ3/4GlcNAc forming H and/or Lewis antigens, more preferentially type II N-acetyllactosamine (Galβ4GlcNAc) forming H type 2, Lewis x, sialyl Lewis x, and/or Lewis y antigens.

LacdiNAc Comprising S1/0H4N5F2/3-Structures

In a preferred embodiment, the invention is directed to analysis of structure of preferred N-glycans with S1/0H4N5F2/3 structures, when the composition comprises biantennary N-glycan type structures with terminal LacdiNAc structure. The LacdiNAc epitope has structure GalNAcβGlcNAc, preferably GalNAcβ4GlcNAc and preferred sialylated LacdiNAc epitope has the structure NeuAcα6GalNAcβ4GlcNAc, based on the known mammalian glycan structure information. Based on biosynthetic knowledge the α6-sialylated structure likely not comprises fucose. The preferred sialyl-lactosamine structures includes NeuAcα3/6Galβ4GlcNAc. The presence of lacdinac structures was revealed by N-acetylhexosaminidase and N-acetylglucosaminidase digestions.

The invention is especially directed to the composition with terminal Lewis x epitope and a sialylated LacdiNAc epitope according to the Formula:

Galβ4(Fucα3)GNβ2Manα3/6(NeuAcα6GalβNAcβ4GNβ2Manα6/3)Manβ4GlcNAcβ4(Fucα6)GN.

The invention is especially directed to the composition with terminal Lewis x epitope and a fucosylated LacdiNAc epitope according to the Formula:

Galβ4(Fucα3)GNβ2Manα3/6(GalβNAcβ4(Fucα3)GNβ2Manα6/3)Manβ4GlcNAcβ4(Fucα6)GN,

and/or structure with Lewis y and LacdiNAc:

Fucα2Galβ4(Fucα3)GNβ2Manα3/6(GalβNAcβ4GNβ2Manα6/3)Manα4GlcNAcβ4(Fucα6)GN.

Multiple N-Acetyllactosamine Comprising Structures

The invention is further directed to multiple (more than 2) N-acetyllactosamine comprising N-glycan structures according to the formulae: S1H7N6F2, S1H7N6F3, S1H6N5F2, S2H6N5F2, and S1H6N5F3.

Preferred Triantennary Glycans

The invention is especially directed to triantennary N-glycans having compositions S1H6N5F2, S2H6N5F2, and S1H6N5F3. Presence of triantennary structures was revealed by specific galactosidase digestions. A preferred type of triantennary N-glycans includes one synthesized by Mgat3. The triantennary N-glycan comprises in a preferred embodiment a core fucose residue. The preferred terminal epitopes include Lewis x, sialyl-Lewis x, H- and Lewis y antigens as described above for biantennary N-glycans.

Preferred Tetraantennary and/or Polylactosamine Structures

The invention is further directed to monosaccharide compositions and glycan corresponding to monosaccharide compositions S1H7N6F2, and S1H7N6F3, which were assigned to correspond to tetra-antennary and/or poly-N-acetyllactosamine epitope comprising N-glycans such as ones with terminal GalβGlcNAcβ3GalβGlcNAcβ-, more preferably type 2 structures Galβ4GlcNAcβ3Galβ4GlcNAcβ-.

hESC-vi, Large Complex-Type N-Glycans

The preferred group includes neutral glycans with compositions H6N5, and H6N5F1.

The preferred structures in this group include:

triantennary N-glycans, in a preferred embodiment the triantennary N-glycan comprises β1,4-linked N-acetyllactosamine, preferably linked to Manα6-arm of the N-glycan (mgat4 product N-glycan) and poly-N-acetyllactosamine elongated biantennary complex-type N-glycans.

hESC-vii, Monoantennary Type N-Glycans

The preferred group includes neutral glycans with compositions including H4N3, and H4N3F1; And preferentially corresponding to structures:

GalβGlcNAcβ2Manα3(Manα6)Manβ4GlcNAcβ4(Fucα6)₀₋₁GlcNAc, more preferentially with type II N-acetyllactosamine antennae, wherein galactose residues are β1,4-linked Galβ4GlcNAcβ2Manα3(Manα6)Manβ4GlcNAcβ4(Fucα6)₀₋₁GlcNAc.

hESC-viii, Terminal HexNAc Complex-Type N-Glycans

The preferred group includes neutral glycans having composition H4N5F3; and sialylated glycans including S2H4N5F1, and S1H4N5F2.

hESC-ix, Elongated Large Complex-Type N-Glycans

The preferred group includes glycans having composition S1H8N7F1, S1H7N6F2, S1H7N6F3, and S1H7N6F1;

preferentially including poly-N-acetyllactosamine sequences.

Terminal Mannose N-Glycans

High Mannose Type Glycans

hESC-iii, High-mannose type N-glycans, including H6N2, H7N2, H8N2, and H9N2.The preferred high Mannose type glycans are according to the formula:

[Mα2]ₙ₁Mα3{[Mα2]ₙ₃Mα6}Mα6{[Mα2]ₙ₆[Mα2]ₙ₇Mα3}Mβ4GNβ4GNyR₂

wherein n1, n3, n6, and n7are either independently 0 or 1;

y is anomeric linkage structure α and/or β or linkage from derivatized anomeric carbon, and

R₂ is reducing end hydroxyl, chemical reducing end derivative or natural asparagine N-glycoside derivative such as asparagine N-glycosides including aminoacid and/or peptides derived from protein;

[ ] indicates determinant either being present or absent depending on the value of n1, n3, n6, n7; and

{ } indicates a branch in the structure;

M is D-Man, GN is N-acetyl-D-glucosamine, y is anomeric structure or linkage type, preferably beta to Asn.

The preferred structures in this group include:

Manα2Manα6(Manα2Manα3)Manα6(Manα2Manα2Manα3)Manβ4GlcNAcβ4GlcNAc

Manα2Manα6([Manα2]₀₋₁Manα3)Manα6([Manα2]₀₋₁Manα2Manα3)Manβ4GlcNAcβ4GlcNAc

hESC-v, Glucosylated high-mannose type N-glycans, including H10N2, H11N2;

preferentially including:

Manα2Manα6(Manα2Manα3)Manα6([Glcα]₀₋₁

GlcαManα2Manα2Manα3)Manβ4GlcNAcβ4GlcNAc

Specific Low Mannose Type Glycan

hESC-iv, Monomannose N-glycan H1N2;

preferentially including the structure Manβ4GlcNAcβ4GlcNAc.

Structures and Compositions Associated with Differentiated Cell Types (EB and St.3)

The invention revealed novel structures present in higher amount in differentiated embryonic stem cells than in corresponding non-differentiated hESCs.

The preferred glycan groups are represented in groups Diff-i to Diff-ix, corresponding to several types of N-glycans. The glycans are preferred in the order from Diff-i to Diff-ix, based on the relative specificity for the non-differentiated hESCs, the differences in the expression are shown in Tables 1 and 2

Terminal Mannose N-glycans

Preferred terminal Low Mannose N-glycans

Diff-i, Low-mannose type N-glycans,

The preferred low mannose glycans have compositions H2N2, H3N2, and H4N2; and fucosylated low-mannose type N-glycans, including H2N2F1, H3N2F1, and H4N2F1.

Several preferred low Man glycans described above can be presented in a Formula:

[Mα3]ₙ₂{[Mα6)]ₙ₄}[Mα6]ₙ₅{[Mα3]ₙ₈}Mβ4GNβ4[∴Fucα5}]ₘGNyR₂

wherein n2, n4, n5, n8, and m are either independently 0 or 1; [ ] indicates determinant being either present or absent depending on the value of n2, n4, n5, n8 and m, { } indicates a branch in the structure;

y and R2 are as indicated for Formula M2.

Preferred non-fucosylated Low mannose N-glycans are according to the Formula:

Mα6Mβ4GNβ4GNyR₂

Mα3Mβ4GNβ4GNyR₂ and

Mα6{Mα3}Mβ4GNβ4GNyR₂.

Mα6Mα6{Mα3}Mβ4GNβ4GNyR₂

Mα3Mα6{Mα3}Mβ4GNβ4GNyR₂

Preferred Individual Structures of Fucosylated Low-Mannose Glycans

Small fucosylated low-mannose structures are especially unusual among known N-linked glycans and form a characteristic glycan group useful for the methods according to the invention, especially analysis and/or separation of cells according to the present invention. These include:

Mβ4GNβ4(Fucα6)GNyR₂

Mα6Mβ4GNβ4(Fucα6)GNyR₂

Mα3Mβ4GNβ4(Fucα6)GNyR₂ and

Mα6Mα6{Mα3}Mβ4GNβ4(Fucα6)GNyR₂ and

Mα3Mα6{Mα3}Mβ4GNβ4(Fucα6)GNyR₂ and

In a specific embodiment the low mannose glycans includes rare structures based on unusual mannosidase degradation Manα2Manα2Manα3Manβ4GNβ4(Fucα6)₀₋₁GN, Manα2Manα3Manβ4GNβ4(Fucα6)₀₋₁GN.

High Mannose Type Glycans

Diff-ii, Fucosylated high-mannose type N-glycans, including H5N2F1, H6N2F1; preferentially including:

Manα6(Manα3)Manα6(Manα3)Manβ4GlcNAcβ4(Fucα6)GlcNAc; and

[Manα2]₀₋₁Manα6([Manα2]₀₋₁Manα3)Manα6(Manα3)Manβ4GlcNAcβ4(Fucα6)GlcNAc

Diff-iii, Small high-mannose type N-glycans, including H5N2, preferably corresponding to the structure

Manα6(Manα3)Manα6(Manα3)Manβ4GlcNAcβ4GlcNAc

Complex Type Glycans

Diff-iv, Terminal HexNAc N-glycans, including H5N6F2, H3N4, H3N5, H4N4F2, H4N5F2, H4N4, H4N5F1, H2N4F1, H3N5F1, and H3N4F1.

The preferred H4H5 structures, H4N5F2 and H4N5F1, include following preferred structures comprising LacdiNAc:

[Fucα]ₙ₃{Gal[NAc]ₙ₁βGNβ2Manα3(Gal[NAc]ₙ₃βGNβ2Manα6)Manβ4GNβ4(Fucα6)ₙ₂GN,

wherein n1 and n2 are either 0 or 1, so that either n1 or n2 is 0 and the other is 1 and n3 is either 0 or 1. The fucose residue forms preferably Lewis x or fucosylated LacdiNAc structure GalNAcβ34(Fucα3)GlcNAc.

Diff-v, Hybrid-type N-glycans, including H5N3F1, H5N3, H6N3F1, and H6N3.

The preferred structures in this group are according to the Formula:

[Galβ]ₙ₁GlcNAcβ2Manα3(Manα3[Manα6]Manα6)Manβ4GlcNAcβ4(Fucα6)ₙ₂GlcNAc

Wherein n1 and n2 are either 0 or 1.

The preferred H5N3 structures are according to the Formula

GlcNAcβ2Manα3(Manα3[Manα6]Manα6)Manβ4GlcNAcβ4(Fucα6)ₙ₂GlcNAc

Wherein n2 is either 0 or 1.

The preferred H6N3 structures are according to the Formula

GalβGlcNAcβ2Manα3(Manα3[Manα6]Manα6)Manβ4GlcNAcβ4(Fucα6)ₙ₂GlcNAc

wherein n2 is either 1 or 0.

Diff-vi, Terminal HexNAc monoantennary N-glycans, including H3N3, H3N3F1, and H2N3F1; preferentially including:

GlcNAcβ2Manα3([Manα6]₀₋₁)Manβ4GlcNAcβ4(Fucα6)₀₋₁GlcNAc, more preferentially with type II N-acetyllactosamine antennae, wherein galactose residues are β1,4-linked.

Diff-vii, H═N type terminal HexNAc N-glycans, including H5N5F1, H5N5, H5N5F3

Terminal HexNAc, especially terminal GlcNAc glycans of this type are described below in more detail.

Diff-viii, Elongated hybrid-type N-glycans, including H6N4, H7N4

GalβGNβ[(]ₙ₁GalβGN[)]ₙ₂β2Manα3([Manα3]ₙ₃[Manα6]ₙ₄Manα6)Manβ4GNβ4GN

n1, and n2 are both either 0 indicating linear structure or 1 indicating a branched structure and n3 and n4 is either 0 or 1, so that at least one is 1. More preferably the structure comprises linear polylactosamine (both n1 and n2 are 0):

GalβGlcNAcβGalβGlcNAcβ2Manα3([Manα3]ₙ₃[Manα6]ₙ₄Manα6)Manβ4GlcNAcβ4GlcNAc,

preferably comprising a β3-linkage between the lactosamines GalβGlcNAcβ3GalβGlcNAc, and even more preferably type 2 N-acetyllactosamines Galβ4GlcNAcβ3Galβ4GlcNAc.

Diff-ix, Complex-fucosylated monoantennary type N-glycans, including H4N3F2;

preferably including:

FucαGalβGlcNAcβ2Manα3([Manβ6]₀₋₁)Manβ4GlcNAcβ4(Fucα6)GlcNAc, preferably the fucose is Fucα2 linked to Gal, or Fucα3/4 linked to GlcNAc;

more preferentially with type II N-acetyllactosamine antennae:

FucαGalβ4GlcNAcβ2Manα3([Manα6]₀₋₁)Manβ4GlcNAcβ4(Fucα6)GlcNAc, even more preferably Fucα2Galβ4GlcNAcβ2Manα3([Manα6]₀₋₁)Manβ4GlcNAcβ4(Fucα6)GlcNAc and/or Galβ4(Fucα3)GlcNAcβ2Manα3([Manα6]₀₋₁)Manβ4GlcNAcβ4(Fucα6)GlcNAc.

Novel Terminal HexNAc N-Glycan Compositions from Stem Cells

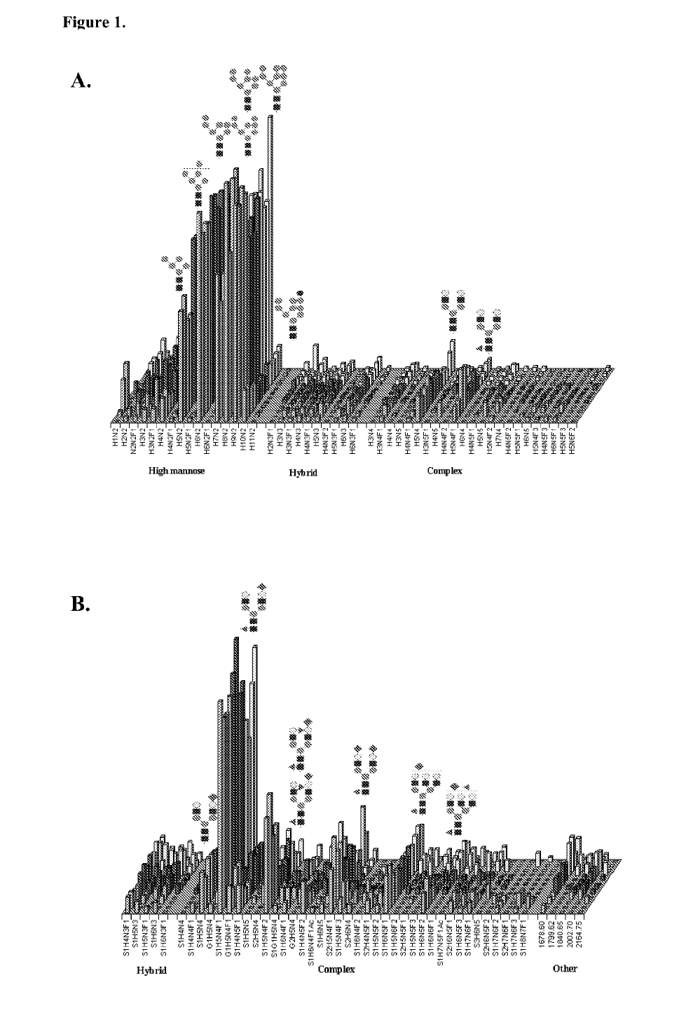

The inventors studied human stem cells as shown in EXAMPLE 1. The data revealed a specific group of altering glycan structures referred as terminal HexNAc structures as shown in Table 5. The FIG. 1 reveals changes of preferred signals in context of differentiation. The terminal HexNAc structures were assigned to include terminal N-acetylglucosamine structures by cleavage with N-acetylglucosamidase enzymes. The Example 2 reveals the analysis of changes of the structures in multiple types of stem cells, the corresponding expression data is summarized in Tables 2 and 3, especially under terminal HexNAc structures.

Preferred N-Glycans According to Structural Subgroups with Terminal HexNAc

The inventors found that there are differentiation stage specific differences with regard to terminal HexNAc containing N-glycans characterized by the formulae: n_{HexNAc}=n_{Hex}≧5 and n_{dHex}≧1 (group I), or: n_{HexNAc}=n_{Hex}≧5 and n_{dHex}=0 (group II). The present data demonstrated that these glycans were 1) detected in various N-glycan samples isolated from both stem cells, including hESC, and cells directly or indirectly differentiated from these cell types; and 2) overexpressed in the analyzed differentiated cells when compared to the corresponding stem cells. There was independent expression between groups I and group II and therefore, the N-glycan structure group determined by the formula n_{HexNAc}=n_{Hex}≧5 is divided into two independently expressed subgroups I and II as described above.

Based on the known specificities of the biosynthetic enzymes synthesizing N-glycan core α1,6-linked fucose and β1,4-linked bisecting GlcNAc, group II preferably corresponds to bisecting GlcNAc type N-glycans while group I preferentially corresponds to other terminal HexNAc containing N-glycans, preferentially with a branching HexNAc in the N-glycan core structure, more preferentially including structures with a branching GlcNAc in the N-glycan core structure. In a specific embodiment the glycan structures of this group includes core fucosylated bisecting GlcNAc comprising N-glycan, wherein the additional GlcNAc is GlcNAcβ4 linked to Manβ4GlcNAc epitope forming epitope structure GlcNAcβ4Manβ4GlcNAc preferably between the complex type N-glycan branches.

In a preferred embodiment of the present invention, such structures include GlcNAc linked to the 2-position of the β1,4-linked mannose. In a further preferred embodiment of the present invention, such structures include GlcNAc linked to the 2-position of the β1,4-linked mannose as described for LEC14 structure (Raju and Stanley J. Biol Chem (1996) 271, 7484-93), this is specifically preferred embodiment, supported by analysis of gene expression data and glycosyltransferase specificities. In a further preferred embodiment of the present invention, such structures include GlcNAc linked to the 6-position of the β1,4-linked GlcNAc of the N-glycan core as described for LEC14 structure (Raju, Ray and Stanley J. Biol Chem (1995) 270, 30294-302).

The invention is specifically directed to further analysis of the subtypes of the group I glycans comprising structures according to the group I. The invention is further directed to production of specific binding reagents against the N-glycan core marker structures and use of these for analysis of the preferred cancer marker structures. The invention is further directed to the analysis of LEC 14 and/or 18 structures by negative recognition by lectins PSA (pisum sativum) or Intil (Lens culinaris) lectin or core Fuc specific monoclonal antibodies, which binding is prevented by the GlcNAcs.

Invention is specifically directed to N-glycan core marker structure, wherein the disaccharide epitope is Manβ4GlcNAc structure in the core structure of N-linked glycan according to the

[Manα3]ₙ₁(Manα6)ₙ₂Manβ4GlcNAcβ4(Fucα6)ₙ₃GlcNAcxR, Formula CGN:
wherein n1, n2 and n3 are integers 0 or 1, independently indicating the presence or absence of the residues, and
wherein the non-reducing end terminal Manα3/Manα6-residues can be elongated to the complex type, especially biantennary structures or to mannose type (high-Man and/or low Man) or to hybrid type structures for the analysis of the status of stem cells and/or manipulation of the stem cells, wherein xR indicates reducing end structure of N-glycan linked to protein or peptide such as βAsn or βAsn-peptide or βAsn-protein, or free reducing end of N-glycan or chemical derivative of the reducing produced for analysis.

The invention is further directed to the N-glycan core marker structure and marker glycan compositions comprising structures of Formula CGN, wherein Manα3/Manα6-residues are elongated to the complex type, especially biantennary structures and n3 is 1 and wherein the Manβ4GlcNAc-epitope comprises the GlcNAc substitutions.

The invention is further directed to the N-glycan core marker structure and marker glycan compositions comprising structures of Formula CGN, wherein Manα3/Manα6-residues are elongated to the complex type, especially biantennary structures and n3 is 1 and wherein the Manβ4GlcNAc-epitope comprises between 1-8% of the GlcNAc substitutions.

The invention is further directed to the N-glycan core marker structure and marker glycan compositions comprising structures of Formula CGN, wherein the structure is selected from the group:

[GlcNAcβ2Manα3](GlcNAcβ2Manα6)Manβ4GlcNAcβ4(Fucα6)ₙ₃GlcNAcxR,

[Galβ4GlcNAcβ2Manα3](Galβ4GlcNAcβ2Manα6)Manβ4GlcNAcβ4(Fucα6)ₙ₃GlcNAcxR,

and sialylated variants thereof when SA is α3 and or α6-linked to one or two Gal residues and Manβ4 or GlcNAcβ4 is substituted by GlcNAc.

The invention is further directed to the N-glycan core marker structure and marker glycan compositions comprising of Formula CGN, wherein the Manβ4GlcNAc-epitope comprises and the GlcNAc residue is β2-linked to Manβ4 forming epitope GlcNAcβ2Manβ4.

The invention is further directed to the N-glycan core marker structure and marker glycan compositions comprising of Formula CGN, wherein the Manβ4GlcNAc-epitope comprises and the GlcNAc residue is 6-linked to GlcNAc of the epitope forming epitope Manβ4(GlcNAc6)GlcNAc.

The invention is further directed to the N-glycan core marker structure and marker glycan compositions comprising of Formula CGN, wherein the Manβ4GlcNAc-epitope comprises and the GlcNAc residue is 4-linked to GlcNAc of the epitope forming epitope GlcNAcβ4Manβ4GlcNAc.

Analysis of Specific Glycan Groups in hESC Glycomes

The analysis of N-glycome revealed signals and monosaccharide compositions specific for embryonic stem cells at various differentiation levels. Some preferred structures are assigned in Tables 12 and 13. The terminal structures were assigned based on specific binding molecules NMR and glycosidase digestions. The binding molecules for terminal epitopes including structures present also in glycolipids or on proteins and lipids are indicated in Tables 14-19. The invention is directed to specific reagents recognizing the preferred terminal epitopes on N-glycans.

Over View of 50 Most Common Structures

Neutral Glycans

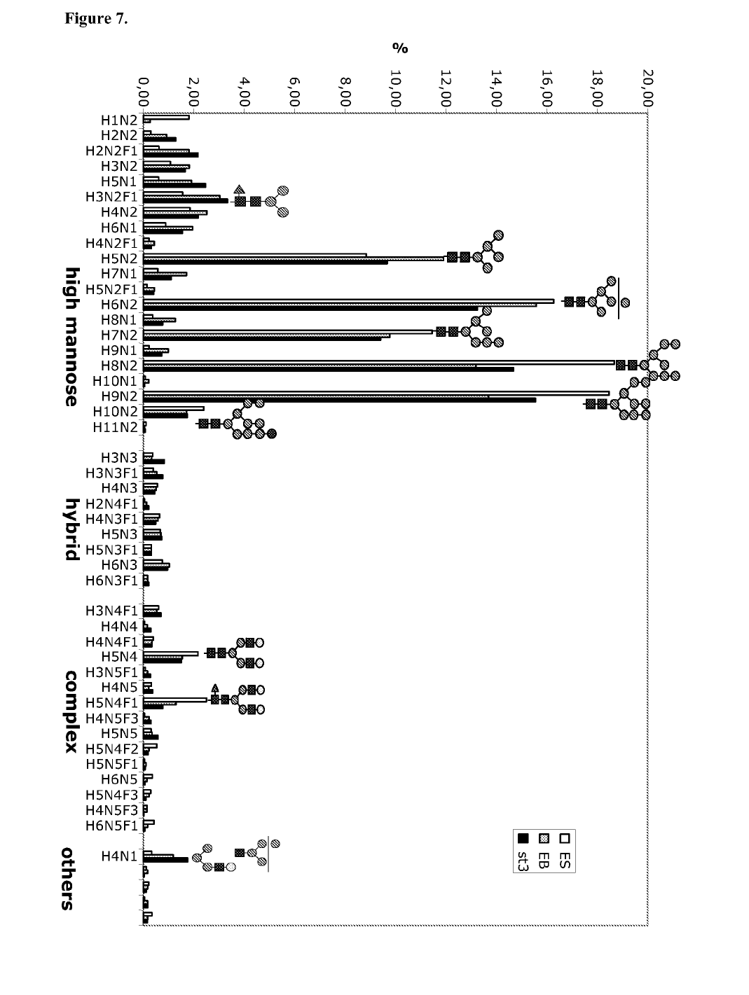

FIG. 7 shows neutral glycans at three differentiation stages. The structures of glycans are indicated by symbols based on the recommendations of Consortium for Functional Glycomics. The glycans include terminal mannose comprising structures with regular high-mannose structures and low mannose structures, with characteristic changes during differentiation.

The mannose glycans further includes single HexNAc comprising structures H₄₋₁₀N₁, which also change during differentiation. A specifically characteristic glycans have compositions H4N1 and H5N1, which increase during differentiation from stage 1 (ES cells) to stage 2 (EB) and further to stage 3. The other signal in this group (H6N1, H7N1, H8N1, H9N1 and H10N1 increase to stage 2 but the decrease.

The glycans are assigned as degradation products of High/Low mannose or even hybrid type structures. A preferred structural assignment is directed to glycans with High/Low mannose structures comprising single GlcNAc unit at the reducing end. This type of glycans have been known from free cytosolic glycans as degradation products of N-glycans. The glycans are produced by endo-beta-N-acetylglucosaminidase (chitobiosidase) cleaving the glycan between the GlcNAc residues. It is realized that the glycan pool may also comprise hybrid type glycans released by endo-beta-mannosidase. The product would comprise N-acetyllactosamine on one branch and mannose residues on the other branch (lower variant of H4N1).

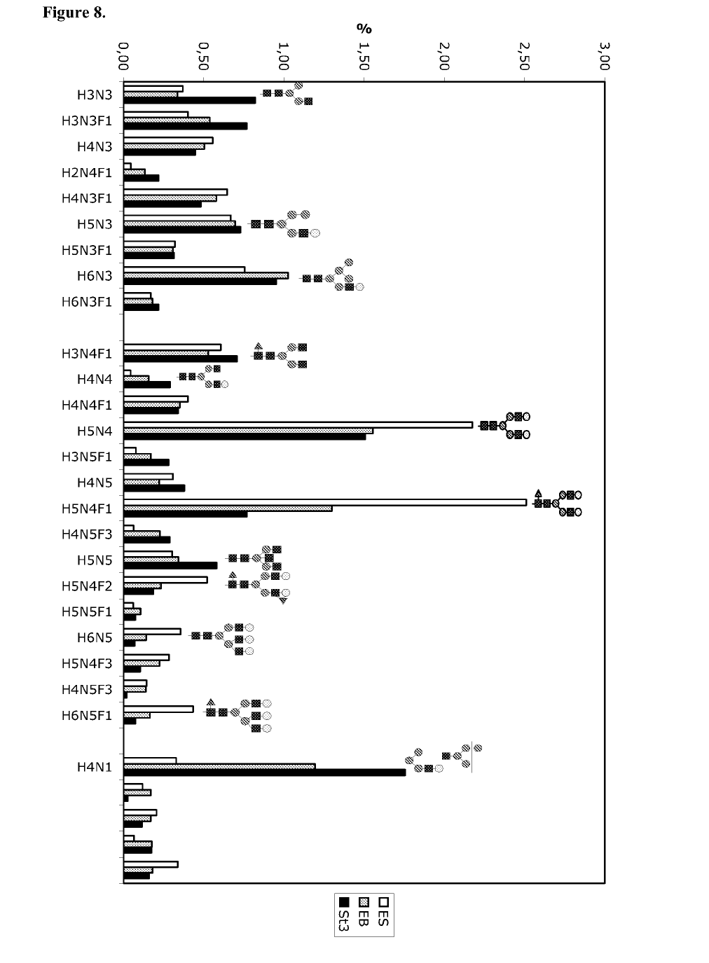

A selection of hybrid and complex type glycans are shown in FIG. 8. The glycans includes hybrid type (and(or monoantennary glycans). In this first group (left) signal H3N3 shows major change from stage 2 to stage 3, and H2N4F1 from stage 1 to stage 3. The glycans classified as complex type structures in the middle also change during differentiation. The major signals corresponding to biantennary N glycans H5N4 and H5N4F1 decrease during the differentiation similarily as difucosylated structure H5N4F2 and multilactosaminylated H6N5 and H6N5F1 structures preferably corresponding to triantennary glycans. The structures increasing during the differentiation includes H4N4, H3N5F1, H4N5F3, and H5N5 (structural scheme is lacking terminal Gal or hexose units).

Acidic Glycans

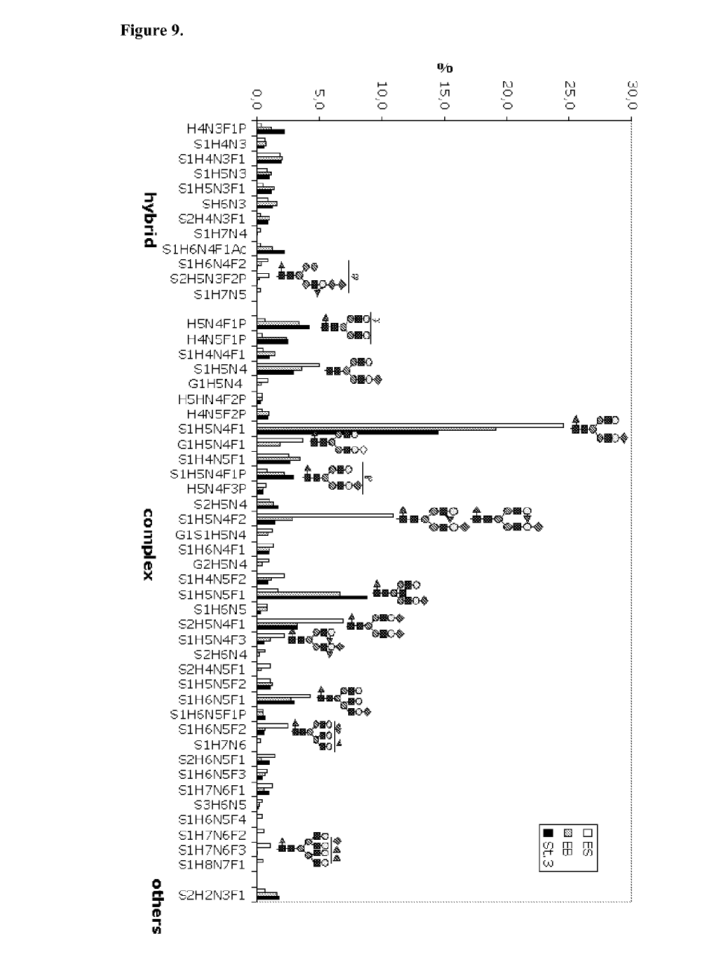

The FIG. 9 indicates 50 most abundant acidic glycans. The major complex type N-glycan signals with sialic acids S1H5N4F1 and S1H5N4F2 decrease during differentiation, while the amounts of sulfated structures H5N4F1P, and S1H5N4F1P (P indicates sulfate or fosfate,) similarily as a structure comprising additional HexNAc (S1H5N5F1) increases.

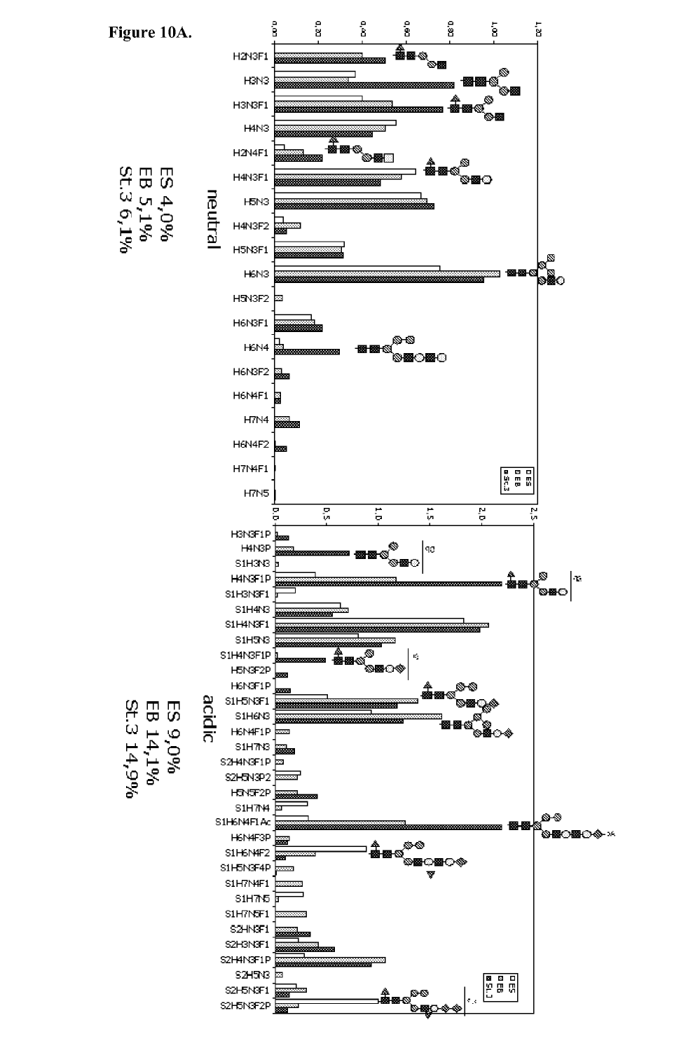

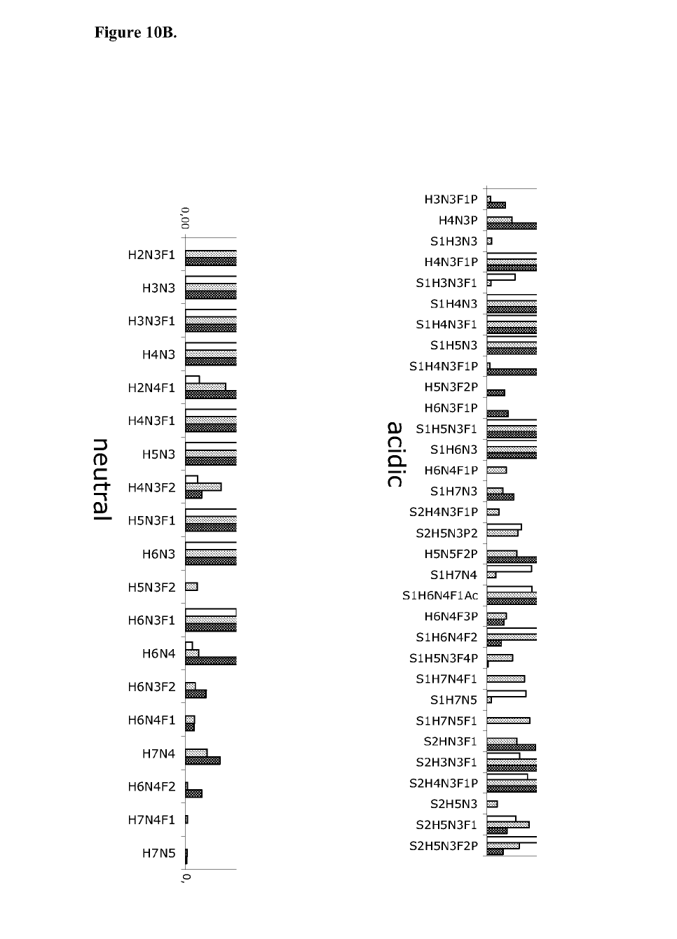

The FIG. 10 shows approximated relative amounts of hybrid type glycans indicating quite similar amounts of acidic and neutral hybrid/monoantennary glycans. The relative amounts of both glycan types increases during differentiation. Sulfated (or fosforylated) glycans are increased among the hybrid type glycans.

The glycans changing during differentiation with composition S1H6N4F1Ac, S 1H6N4F2, and H6N4 in a specific embodiment include biantennary structures with additional terminal hexose, which may be derived from exogenous proteins, in a specific embodiment the hexose is Galα3-structure.

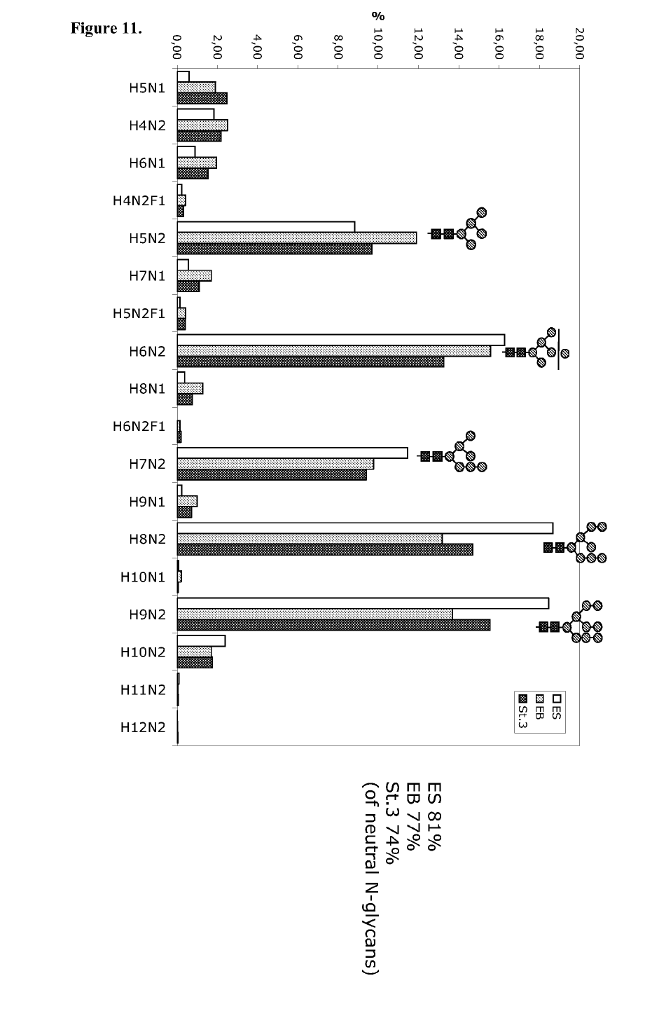

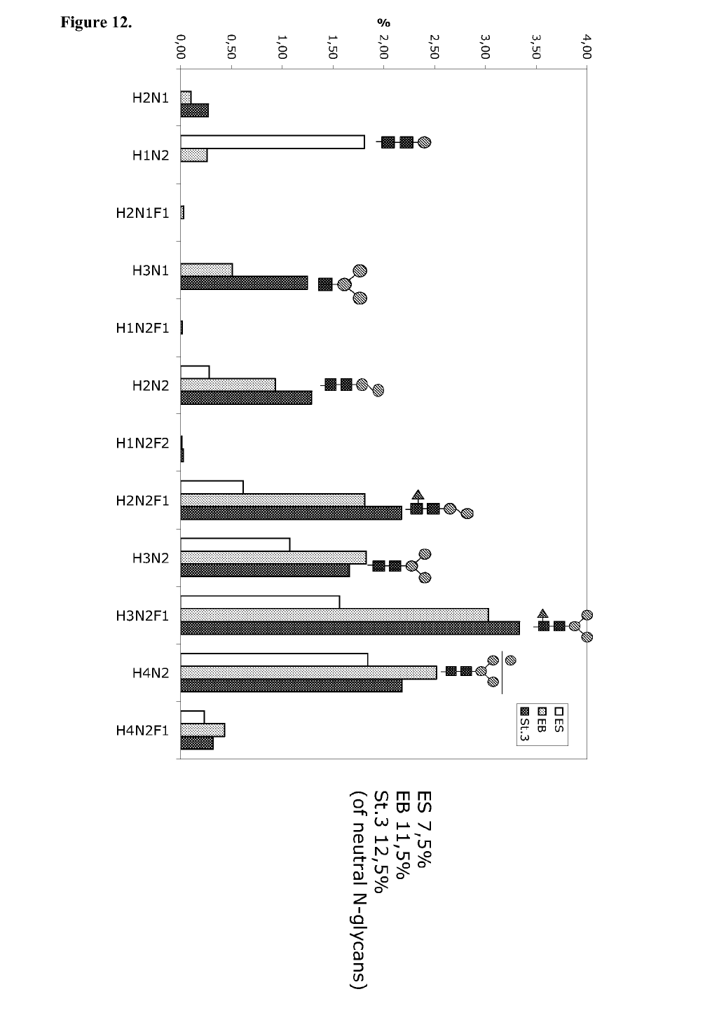

FIGS. 11 and 12 includes high and Low mannose structures. The changes of the low mannose structures during the differentiation are characteristic for the stem cells. The smallest low mannose structure (H1N2) decreases while larger ones increase.

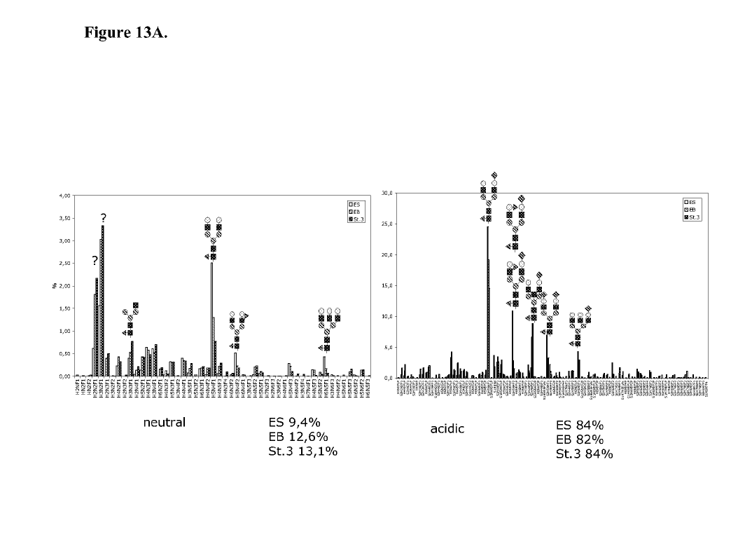

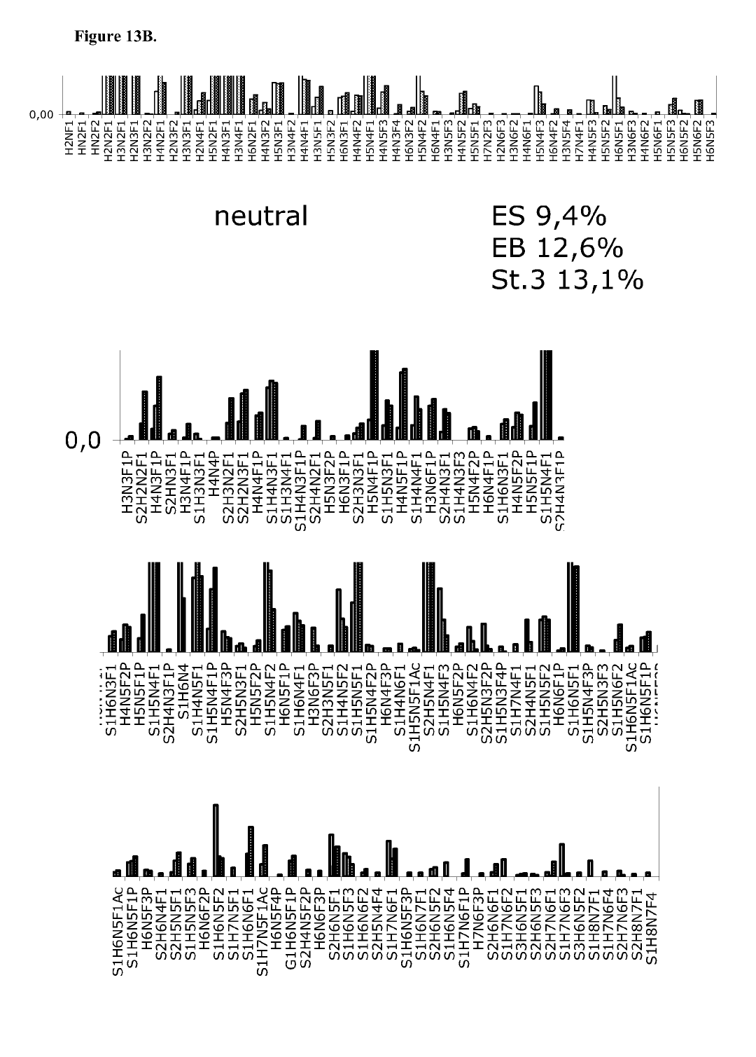

Neutral and acidic fucosylated glycans are presented in FIG. 13 Among the entral fucosylated glycans the amounts of apparently degraded low mannose group structures are increased (H2N2F1, H3N2F1 and H3N3F1), while the complex type structures decrease similarily in acidic and neutral glycans except the structure with additional HexNAc, S1H5N5F1.

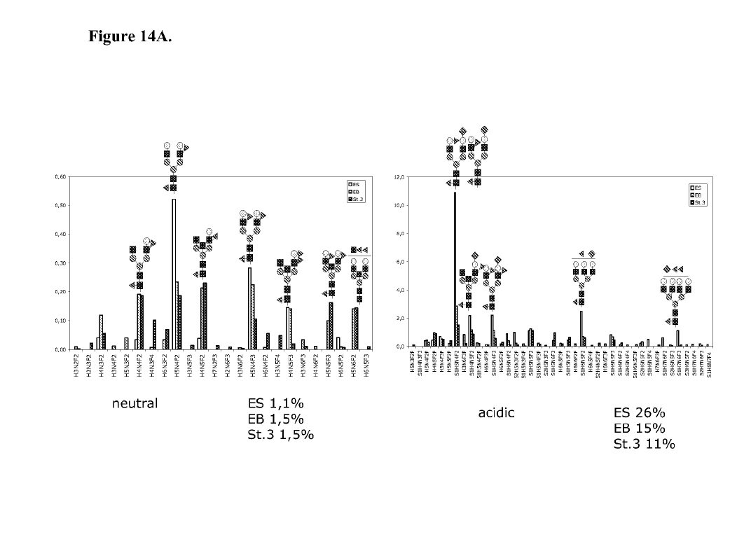

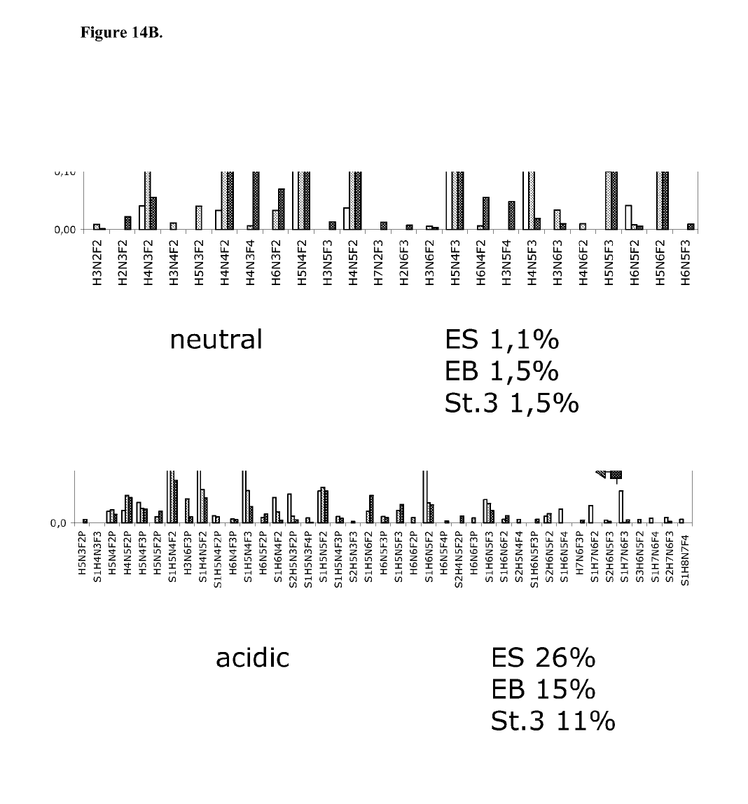

FIG. 14 shows the neutral and acidic glycans comprising at least two fucose residues. These are considered as comprising fucosylated lactosamine and referred as complex/complexly fucosylated structures. In general decrease of the complexly fucosylated structures is observed except the structures with additional HexNAc residues, H4N4F2 (potential degradation product), H5N5F3, H5N6F3.

Preferred Sulfated Marker Structures in N-Glycome of Embryonic Stem Cells

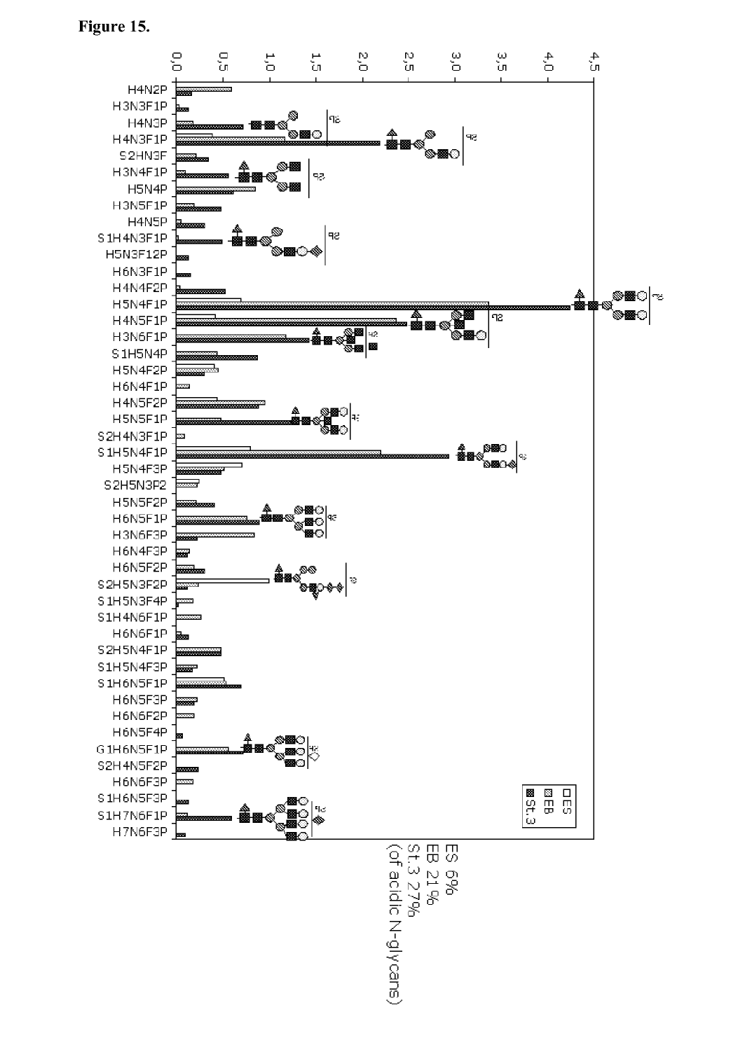

FIG. 15 represents sulfated N-glycans of human embryonic stem cells and changes in their relative abundance during differentiation. There is major changes during differentiation. The invention is directed to use of the signals, monosaccharide compositions and structures indicated as increasing in FIG. 15 for markers of differentiating embryonic stem cells. Experiments by cleavage by specific fosfatase enzyme and high resolution mass spectrometry indicate that the structures with complex type N-glycans with N-acetyllactosamine residues preferably carry sulfate residues (sulfate ester structures) and the Mannose type N-glycans such as high Mannose N-glycans preferably carries fosfate residue(s). It is realised that the sulphated and/or fosforylated glycomes from stem cells are new inventive markers.

The invention is especially directed to the recognition of sulphated N-acetyllactosamines as differentiation markers of stem cells, embryonic stem cells. The invention is directed to testing and selectin optimal stem cell recognizing binder molecule, preferably antibodies such as monoclonal antibodies, recognizing preferred sulphated lactosamines including type 1 (Galβ3GlcNAc) and type II lactosamines (Galβ4GlcNAc) comprising sulfate residue(ester) at either position 3 or 6 of Gal and/or on position 6 of GlcNAc. The invention is especially directed to the recognition of the sulphated lactosamines from an N-glycan composition as shown by the invention.

Large N-Glycan Structure

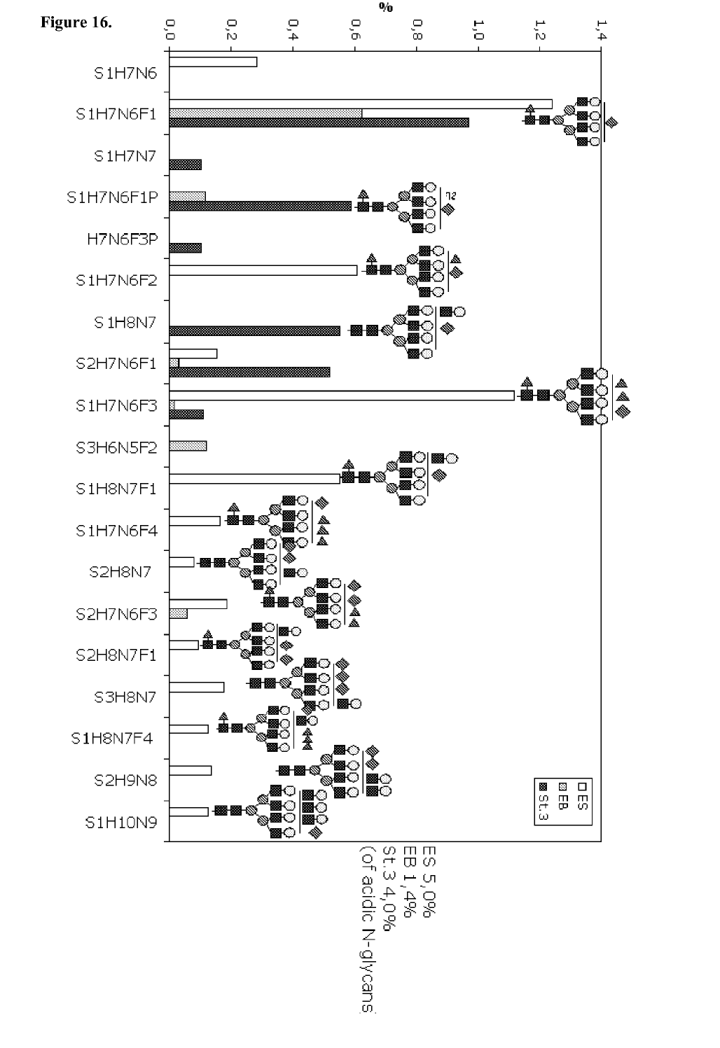

FIG. 16. shows large N-glycans (H≧7, N≧6) of human embryonic stem cells and changes in their relative abundance during differentiation. FIG. 16 represents large N-glycans of human embryonic stem cells and changes in their relative abundance during differentiation. There is major changes during differentiation. The invention is directed to use of the signals, monosaccharide compositions and structures indicated as increasing in FIG. 16 for markers of differentiating embryonic stem cells.

The invention reveals that the N-glycans of embryonic stem cells comprise multiantennary N-aglycans with at least three antennae with characteristic differentiation associated cahges. The invention reveals even much larger N-glycans contain poly-N-acetyllctosamine glycans. The invention is especially directed to use of reagents recognizing linear (example of preferred regent potato lectin, Solanum tuberosum agglutinin, STA) or branched poly-N-acetyllactosamine. The results revealed that recognition of branched N-acetyllactosamines is especially useful for characterization or separation or manipulation of embyronal stem cells. Preferred reagents includes PWA, pokeweed agglutinin and/or antibody recognizing branched poly-N-acetyllactosamines such as I-blood group antibodies.

Cell Types

In the present text, cell types refer to stem cells, especially human embryonic stem cells (hESC) and cells differentiated from them, preferentially embryoid bodies (EB) and stage 3 (st.3) and further differentiated cells.

Glycan Dataset and Glycan Profile Analysis

The present invention is directed to analysing glycan profiles to enable uses including the following:
1. comparison between stem cell and differentiated samples,
2. comparison between different samples of the same cell type,
3. identification of differentiation stage,
4. identification of glycan signals and glycan structures associated with different cell types or differentiation stages,
5. identification of glycan signal groups and glycan structure groups associated with different cell types or differentiation stages,
6. identification of biosynthetic glycan groups associated with different cell types or differentiation stages,
7. identification of glycan fingerprints and glycan signatures, i.e. glycan profiles or subprofiles therefrom, respectively, which are associated with different cell types or differentiation stages, and
8. evaluating glycans or glycan groups with respect to their degree of association with given cell type.

As described in the present invention, analysis of multiple samples from the same cell type reveals that some glycans or glycan groups are constantly associated with given cell type, whereas other glycans or glycan groups vary individually or between different samples within the same cell type. The present invention is especially directed to analyzing multiple samples of a given cell type to reach a point of statistical confidence, preferentially over 95% confidence level and even more preferentially over 96% confidence level, where given cell type or the glycan types associated with it can be reliably identified.

The present invention is specifically directed to comparison of multiple glycan profile data to find out which glycan signals are consistently associated with given cell type or not present in it, which are constant in all cell types, which are subject to individual or cell line specific variation, and which are indicative for the absence or presence of certain differentiation stages or lineages, more preferentially pluripotency (stem cell) or neuroectodermal differentation. The inventors found that the N-glycan profiles of human embryonic stem cells and cell derived from them contain glycan signals and glycan signal groups with the properties described above.

The present invention is further directed to establishing reference datasets from single glycan signals or glycan fingerprints or signatures (profiles or subprofiles), which can be reliably used for quality control, estimation of differential properties of new samples, control of variation between samples, or estimation of the effects of external factors or culture conditions on cell status. In this aspect of the invention, data acquired from new sample are compared to reference dataset with a predetermined equation to evaluate the status of the sample.

Structure Specific Glycan Binding Reagents

The present invention is further directed to using knowledge of glycan features associated with different cell types or differentiation stages to design glycan-binding reagents, more preferably glycan-binding proteins, for specific identification of stem cells or differentiated cells. The present invention is further directed to using such structure specific reagents to specifically recognize, label, or tag either specific stem cell or specific differentiated cell types, more preferentially animal feeder cells and more preferably mouse feeder cells. Such labels or tags can then be used to isolate and/or remove such cells by methods known in the art.

The Binding Methods for Recognition of Structures from Cell Surfaces

Recognition of Structures from Glycome Materials and on Cell Surfaces by Binding Methods

The present invention revealed that beside the physicochemical analysis by NMR and/or mass spectrometry several methods are useful for the analysis of the structures. The invention is especially directed to two methods:
i) Recognition by enzymes involving binding and alteration of structures.
This method alters specific glycan structures by enzymes capable of altering the glycan structures. The preferred enzymes includes
   a) glycosidase-type enzymes capable of releasing monosaccharide units from glycans
   b) glycosyltransferring enzymes, including transglycosylating enzymes and glycosyltransferases
   c) glycan modifying enzymes including sulfate and or fosfate modifying enzymes
ii) Recognition by molecules binding glycans referred as the binders
These molecules bind glycans and include property allowing observation of the binding such as a label linked to the binder. The preferred binders include
   a) Proteins such as antibodies, lectins and enzymes
   b) Peptides such as binding domains and sites of proteins, and synthetic library derived analogs such as phage display peptides
   c) Other polymers or organic scaffold molecules mimicking the peptide materials

The peptides and proteins are preferably recombinant proteins or corresponding carbohydrate recognition domains derived therereof, when the proteins are selected from the group monoclonal antibody, glycosidase, glycosyl transferring enzyme, plant lectin, animal lectin or a peptide mimetic thereof, and wherein the binder includes a detectable label structure.

Preferred Binder Molecules

The present invention revealed various types of binder molecules useful for characterization of cells according to the invention and more specifically the preferred cell groups and cell types according to the invention. The preferred binder molecules are classified based on the binding specificity with regard to specific structures or structural features on carbohydrates of cell surface. The preferred binders recognize specifically more than single monosaccharide residue.

It is realized that most of the current binder molecules such as all or most of the plant lectins are not optimal in their specificity and usually recognize roughly one or several monosaccharides with various linkages. Furthermore the specificities of the lectins are usually not well characterized with several glycans of human types.

The preferred high specificity binders recognize
A) at least one monosaccharide residue and a specific bond structure between those to another monosaccharides next monosaccharide residue referred as MS1B1-binder,
B) more preferably recognizing at least part of the second monosaccharide residue referred as MS2B1-binder,
C) even more preferably recognizing second bond structure and or at least part of third mono saccharide residue, referred as MS3B2-binder, preferably the MS3B2 recognizes a specific complete trisaccharide structure.
D) most preferably the binding structure recognizes at least partially a tetrasaccharide with three bond structures, referred as MS4B3-binder, preferably the binder recognizes complete tetrasaccharide sequences.

The preferred binders includes natural human and or animal, or other proteins developed for specific recognition of glycans. The preferred high specificity binder proteins are specific antibodies preferably monoclonal antibodies; lectins, preferably mammalian or animal lectins; or specific glycosyltransferring enzymes more preferably glycosidase type enzymes, glycosyltransferases or transglycosylating enzymes.

Target Structures for Specific Binders and Examples of the Binding Molecules

Combination of Terminal Structures in Combination with Specific Glycan Core Structures

It is realized that part of the structural elements are specifically associated with specific glycan core structure. The recognition of terminal structures linked to specific core structures are especially preferred, such high specificity reagents have capacity of recognition almost complete individual glycans to the level of physicochemical characterization according to the invention. For example many specific mannose structures according to the invention are in general quite characteristic for N-glycan glycomes according to the invention. The present invention is especially directed to recognition terminal epitopes.

Common Terminal Structures on Several Glycan Core Structures

The present invention revealed that there are certain common structural features on several glycan types and that it is possible to recognize certain common epitopes on different glycan structures by specific reagents when specificity of the reagent is limited to the terminal without specificity for the core structure. The invention especially revealed characteristic terminal features for specific cell types according to the invention. The invention realized that the common epitopes increase the effect of the recognition. The common terminal structures are especially useful for recognition in the context with possible other cell types or material, which do not contain the common terminal structure in substantial amount.

Specific Preferred Structural Groups

The present invention is directed to recognition of oligosaccharide sequences comprising specific terminal monosaccharide types, optionally further including a specific core structure. The preferred oligosaccharide sequences classified based on the terminal monosaccharide structures.

1. Structures with Terminal Mannose Monosaccharide

Preferred mannose-type target structures have been specifically classified by the invention. These include various types of high and low-mannose structures and hybrid type structures according to the invention.

Low or Uncharacterised Specificity Binders

preferred for recognition of terminal mannose structures includes mannose-monosaccharide binding plant lectins.

Preferred High Specific High Specificity Binders

include

i) Specific mannose residue releasing enzymes such as linkage specific mannosidases, more preferably an α-mannosidase or β-mannosidase.

Preferred α-mannosidases includes linkage specific α-mannosidases such as α-Mannosidases cleaving preferably non-reducing end terminal

α2-linked mannose residues specifically or more effectively than other linkages, more preferably cleaving specifically Manα2-structures; or

α6-linked mannose residues specifically or more effectively than other linkages, more preferably cleaving specifically Manα6-structures;

Preferred β-mannosidases includes β-mannosidases capable of cleaving β4-linked mannose from non-reducing end terminal of N-glycan core Manβ4GlcNAc-structure without cleaving other β-linked monosaccharides in the glycomes.

ii) Specific binding proteins recognizing preferred mannose structures according to the invention.

The preferred reagents include antibodies and binding domains of antibodies (Fab-fragments and like), and other engineered carbohydrate binding proteins. The invention is directed to antibodies recognizing MS2B1 and more preferably MS3B2-structures

2. Structures with Terminal Gal-Monosaccharide

Preferred galactose-type target structures have been specifically classified by the invention. These include various types of N-acetyllactosamine structures according to the invention.

Low or Uncharacterised Specificity Binders for Terminal Gal

Prereferred for recognition of terminal galactose structures includes plant lectins such as ricin lectin (ricinus communis agglutinin RCA), and peanut lectin(/agglutinin PNA).

Preferred High Specific High Specificity Binders Include

i) Specific galactose residue releasing enzymes such as linkage specific galactosidases, more preferably α-galactosidase or β-galactosidase.

Preferred α-galactosidases include linkage galactosidases capable of cleaving Galα3Gal-structures revealed from specific cell preparations

Preferred β-galactosidases includes β-galactosidases capable of cleaving

β4-linked galactose from non-reducing end terminal Galβ4GlcNAc-structure without cleaving other β-linked monosaccharides in the glycomes and

β3-linked galactose from non-reducing end terminal Galβ3GlcNAc-structure without cleaving other β-linked monosaccharides in the glycomes

ii) Specific binding proteins recognizing preferred galactose structures according to the invention.

The preferred reagents include antibodies and binding domains of antibodies (Fab-fragments and like), and other engineered carbohydrate binding proteins and animal lectins such as galectins.

3. Structures with Terminal GalNAc-Monosaccharide

Preferred GalNAc-type target structures have been specifically revealed by the invention. These include especially LacdiNAc, GalNAcβGlcNAc-type structures according to the invention.

Low or Uncharacterised Specificity Hinders for Terminal GalNAc

Several plant lectins has been reported for recognition of terminal GalNAc. It is realized that some GalNAc-recognizing lectins may be selected for low specificity recognition of the preferred LacdiNAc-structures.

Preferred High Specific High Specificity Binders Include

i) The invention revealed that β-linked GalNAc can be recognized by specific β-N-acetylhexosaminidase enzyme in combination with β-N-acetylhexosaminidase enzyme.

This combination indicates the terminal monosaccharide and at least part of the linkage structure.

Preferred β-N-acetylehexosaminidase, includes enzyme capable of cleaving β-linked GalNAc from non-reducing end terminal GalNAcβ4/3-structures without cleaving α-linked HexNAc in the glycomes; preferred N-acetylglucosaminidases include enzyme capable of cleaving β-linked GlcNAc but not GalNAc.

ii) Specific binding proteins recognizing preferred GalNAcβ4, more preferably GalNAcβ4GlcNAc, structures according to the invention. The preferred reagents include antibodies and binding domains of antibodies (Fab-fragments and like), and other engineered carbohydrate binding proteins, and a special plant lectin WFA (Wisteria floribunda agglutinin).

4. Structures with Terminal GlcNAc-Monosaccharide

Preferred GlcNAc-type target structures have been specifically revealed by the invention. These include especially GlcNAcβ-type structures according to the invention.

Low or Uncharacterised Specificity Binders for Terminal GlcNAc

Several plant lectins has been reported for recognition of terminal GlcNAc. It is realized that some GlcNAc-recognizing lectins may be selected for low specificity recognition of the preferred GlcNAc-structures.

Preferred High Specific High Specificity Binders Include

i) The invention revealed that β-linked GlcNAc can be recognized by specific β-N-acetylglucosaminidase enzyme.

Preferred β-N-acetylglucosaminidase includes enzyme capable of cleaving β-linked GlcNAc from non-reducing end terminal GlcNAcβ2/3/6-structures without cleaving β-linked GalNAc or α-linked HexNAc in the glycomes;

ii) Specific binding proteins recognizing preferred GlcNAcβ2/3/6, more preferably GlcNAcβ2Manα, structures according to the invention. The preferred reagents include antibodies and binding domains of antibodies (Fab-fragments and like), and other engineered carbohydrate binding proteins.

5. Structures with Terminal Fucose-Monosaccharide

Preferred fucose-type target structures have been specifically classified by the invention. These include various types of N-acetyllactosamine structures according to the invention.

Low or Uncharacterised Specificity Hinders for Terminal Fuc

Prereferred for recognition of terminal fucose structures includes fucose monosaccharide binding plant lectins. Lectins of Ulex europeaus and Lotus tetragonolobus has been reported to recognize for example terminal Fucoses with some specificity binding for α2-linked structures, and branching α3-fucose, respectively.

Preferred High Specific High Specificity Binders Include

i) Specific fucose residue releasing enzymes such as linkage fucosidases, more preferably α-fucosidase.

Preferred α-fucosidases include linkage fucosidases capable of cleaving Fucα2Gal-, and Galβ4/3(Fucα3/4)GlcNAc-structures revealed from specific cell preparations.

ii) Specific binding proteins recognizing preferred fucose structures according to the invention. The preferred reagents include antibodies and binding domains of antibodies (Fab-fragments and like), and other engineered carbohydrate binding proteins and animal lectins such as selectins recognizing especially Lewis type structures such as Lewis x, Galβ4(Fucα3)GlcNAc, and sialyl-Lewis x, SAα3Galβ4(Fucα3)GlcNAc.

The preferred antibodies includes antibodies recognizing specifically Lewis type structures such as Lewis x, and sialyl-Lewis x. More preferably the Lewis x-antibody is not classic SSEA-1 antibody, but the antibody recognizes specific protein linked Lewis x structures such as Galβ4(Fucα3)GlcNAcβ2Manα-linked to N-glycan core.

6. Structures with Terminal Sialic Acid-Monosaccharide

Preferred sialic acid-type target structures have been specifically classified by the invention.

Low or Uncharacterised Specificity Binders for Terminal Fuc

Preferred for recognition of terminal sialic acid structures includes sialic acid monosaccharide binding plant lectins.

Preferred High Specific High Specificity Binders Include

i) Specific sialic acid residue releasing enzymes such as linkage sialidases, more preferably α-sialidases.

Preferred α-sialidases include linkage sialidases capable of cleaving SAα3Gal- and SAα6Gal-structures revealed from specific cell preparations by the invention.

Preferred lectins, with linkage specificity include the lectins, that are specific for SAα3Gal-structures, preferably being Maackia amurensis lectin and/or lectins specific for SAα6Gal-structures, preferably being Sambucus nigra agglutinin.

ii) Specific binding proteins recognizing preferred sialic acid oligosaccharide sequence structures according to the invention. The preferred reagents include antibodies and binding domains of antibodies (Fab-fragments and like), and other engineered carbohydrate binding proteins and animal lectins such as selectins recognizing especially Lewis type structures such as sialyl-Lewis x, SAα3Galβ4(Fucα3)GlcNAc or sialic acid recognizing Siglec-proteins.

The preferred antibodies includes antibodies recognizing specifically sialyl-N-acetyllactosamines, and sialyl-Lewis x.

Preferred antibodies for NeuGc-structures includes antibodies recognizes a structure NeuGcα3Galβ4Glc(NAc)_{0 or 1} and/or GalNAcβ4[NeuGcα3]Galβ4Glc(NAc)_{0 or 1}, wherein [ ] indicates branch in the structure and ( )_{0 or 1} a structure being either present or absent. In a preferred embodiment the invention is directed recognition of the N-glycolyl-Neuraminic acid structures by antibody, preferably by a monoclonal antibody or human/humanized monoclonal antibody. A preferred antibody contains the variable domains of P3-antibody.

Binder-Label Conjugates

The present invention is specifically directed to the binding of the structures according to the present invention, when the binder is conjugated with “a label structure”. The label structure means a molecule observable in a assay such as for example a fluorescent molecule, a radioactive molecule, a detectable enzyme such as horse radish peroxidase or biotin/streptavidin/avidin. When the labelled binding molecule is contacted with the cells according to the invention, the cells can be monitored, observed and/or sorted based on the presence of the label on the cell surface. Monitoring and observation may occur by regular methods for observing labels such as fluorescence measuring devices, microscopes, scintillation counters and other devices for measuring radioactivity.

Use of Binder and Labelled Binder-Conjugates for Cell Sorting

The invention is specifically directed to use of the binders and their labelled conjugates for sorting or selecting cells from biological materials or samples including cell materials comprising other cell types. The preferred cell types includes cultivated cells and associated cells such as feeder cells. The labels can be used for sorting cell types according to invention from other similar cells. In another embodiment the cells are sorted from different cell types such as blood cells or in context of cultured cells preferably feeder cells, for example in context of complex cell cultures corresponding feeder cells such as human or mouse feeder cells. A preferred cell sorting method is FACS sorting. Another sorting methods utilized immobilized binder structures and removal of unbound cells for separation of bound and unbound cells.

Use of Immobilized Binder Structures

In a preferred embodiment the binder structure is conjugated to a solid phase. The cells are contacted with the solid phase, and part of the material is bound to surface. This method may be used to separation of cells and analysis of cell surface structures, or study cell biological changes of cells due to immobilization. In the analytics involving method the cells are preferably tagged with or labelled with a reagent for the detection of the cells bound to the solid phase through a binder structure on the solid phase. The methods preferably further include one or more steps of washing to remove unbound cells.

Preferred solid phases include cell suitable plastic materials used in contacting cells such as cell cultivation bottles, petri dishes and microtiter wells; fermentor surface materials

Specific Recognition Between Preferred Stem Cells and Contaminating Cells

The invention is further directed to methods of recognizing stem cells from differentiated cells such as feeder cells, preferably animal feeder cells and more preferably mouse feeder cells. It is further realized, that the present reagents can be used for purification of stem cells by any fractionation method using the specific binding reagents.

Preferred fractionation methods includes fluorecense activated cell sorting (FACS), affinity chromatography methods, and bead methods such as magnetic bead methods.

Preferred reagents for recognition between preferred cells, preferably embryonic type cells, and and contaminating cells, such as feeder cells most preferably mouse feeder cells, includes reagents according to the Table 43, more preferably proteins with similar specificity with lectins PSA, MAA, and PNA.

The invention is further directed to positive selection methods including specific binding to the stem cell population but not to contaminating cell population. The invention is further directed to negative selection methods including specific binding to the contaminating cell population but not to the stem cell population. In yet another embodiment of recognition of stem cells the stem cell population is recognized together with a homogenous cell population such as a feeder cell population, preferably when separation of other materials is needed. It is realized that a reagent for positive selection can be selected so that it binds stem cells as in present invention and not to the contaminating cell population and a regent for negative selection by selecting opposite specificity. In case of one population of cells according to the invention is to be selected from a novel cell population not studied in the present invention, the binding molecules according to the invention maybe used when verified to have suitable specificity with regard to the novel cell population (binding or not binding). The invention is specifically directed to analysis of such binding specificity for development of a new binding or selection method according to the invention.

The preferred specificities according to the invention includes recognition of:
i) mannose type structures, especially alpha-Man structures like lectin PSA, preferably on the surface of contaminating cells
ii) α3-sialylated structures similarity as by MAA-lectin, preferably for recognition of embryonic type stem cells
iii) Gal/GalNAc binding specificity, preferably Gal1-3/GalNAc1-3 binding specificity, more preferably Galβ1-3/GalNAcβ1-3 binding specificity similar to PNA, preferably for recognition of embryonic type stem cells

Manipulation of Cells by Binders

The invention is specifically directed to manipulation of cells by the specific binding proteins. It is realized that the glycans described have important roles in the interactions between cells and thus binders or binding molecules can be used for specific biological manipulation of cells. The manipulation may be performed by free or immobilized binders. In a preferred embodiment cells are used for manipulation of cell under cell culture conditions to affect the growth rate of the cells.

Identification and Classification of Differences in Glycan Datasets

The present invention is specifically directed to analyzing glycan datasets and glycan profiles for comparison and characterization of different cell types. In one embodiment of the invention, glycan signals or signal groups associated with given cell type are selected from the whole glycan datasets or profiles and indifferent glycan signals are removed. The resulting selected signal groups have reduced background and less observation points, but the glycan signals most important to the resolving power are included in the selection. Such selected signal groups and their patterns in different sample types serve as a signature for the identification of the cell type and/or glycan types or biosynthetic groups that are typical to it. By evaluating multiple samples from the same cell type, glycan signals that have individual i.e. cell line specific variation can be excluded from the selection. Moreover, glycan signals can be identified that do not differ between cell types, including major glycans that can be considered as housekeeping glycans.

To systematically analyze the data and to find the major glycan signals associated with given cell type according to the invention, difference-indicating variables can be calculated for the comparison of glycan signals in the glycan datasets. Preferential variables between two samples include variables for absolute and relative difference of given glycan signal between the datasets from two cell types. Most preferential variables according to the invention are:

absolute difference A=(S2−S1), and 1.

relative difference R=A/S1, 2.

wherein S1 and S2 are relative abundances of a given glycan signal in cell types 1 and 2, respectively.

It is realized that other mathematical solutions exist to express the idea of absolute and relative difference between glycan datasets, and the above equations do not limit the scope of the present invention. According to the present invention, after A and R are calculated for the glycan profile datasets of the two cell types, the glycan signals are thereafter sorted according to the values of A and R to identify the most significant differing glycan signals. High value of A or R indicates association with cell type 2, and vice versa. In the list of glycan data sorted independently by R and A, the cell-type specific glycans occur at the top and the bottom of the lists. More preferentially, if a given signal has high values of both A and R, it is more significant.

Preferred Representation of the Dataset when Comparing Two Cell Materials

The present invention is specifically directed to the comparative presentation of the quantitative glycome dataset as multidimensional graphs comparing the paraller data for example as shown in figures or as other three dimensional presentations as for example as two dimensional matrix showing the quantities with a quantitative code, preferably by a quantitative color code.

Released Glycomes

The invention is directed to methods to produce released, in a preferred enzymatically released glycans, also referred as glycomes, from embryonic type cells. A preferred glycome type is N-glycan glycome released by a N-glycosidase enzyme. The invention is further directed to profiling analysis of the released glycomes.

Low Amounts of Cells for Glycome Analysis from Stem Cells

The invention revealed that its possible to produce glycome from very low amount of cells. The preferred embodiments amount of cells is between 1000 and 10 000 000 cells, more preferably between 10 000 and 1 000 000 cells. The invention is further directed to analysis of released glycomes of amount of at least 0.1 pmol, more preferably of at least to 1 pmol, more preferably at least of 10 pmol.

(a) Total asparagine-linked glycan (N-glycan) pool was enzymatically isolated from about 100 000 cells. (b) The total N-glycan pool (picomole quantities) was purified with microscale solid-phase extraction and divided into neutral and sialylated N-glycan fractions. The N-glycan fractions were analyzed by MALDI-TOF mass spectrometry either in positive ion mode for neutral N-glycans (c) or in negative ion mode for sialylated glycans (d). Over one hundred N-glycan signals were detected from each cell type revealing the surprising complexity of hESC glycosylation. The relative abundances of the observed glycan signals were determined based on relative signal intensities (Saarinen et al., 1999, Eur. J. Biochem. 259, 829-840).

Preferred Structures of O-Glycan Glycomes of Stem Cells

The present invention is especially directed to following O-glycan marker structures of stem cells: Core 1 type O-glycan structures following the marker composition NeuAc₂Hex₁HexNAc₁, preferably including structures SAα3Galβ3GalNAc and/or SAα3Galβ3(Saα6)GalNAc; and Core 2 type O-glycan structures following the marker composition NeuAc₀₋₂Hex₂HexNAc₂dHex₀₋₁, more preferentially further including the glycan series NeuAc₀₋₂Hex₂₊ₙHexNAc₂₊ₙdHex₀₋₁, wherein n is either 1, 2, or 3 and more preferentially n is 1 or 2, and even more preferentially n is 1;

more specifically preferably including R₁Galβ4(R₃)GlcNAcβ6(R₂Galβ3)GalNAc,

wherein R₁ and R₂ are independently either nothing or sialic acid residue, preferably α2,3-linked sialic acid residue, or an elongation with HexₙHexNAcₙ, wherein n is independently an integer at least 1, preferably between 1-3, most preferably between 1-2, and most preferably 1, and the elongation may terminate in sialic acid residue, preferably α2,3-linked sialic acid residue; and R₃ is independently either nothing or fucose residue, preferably a1,3-linked fucose residue.

It is realized that these structures correlate with expression of β6GlcNAc-transferases synthesizing core 2 structures.

Preferred Branched N-Acetyllactosamine Type Glycosphingolipids

The invention further revealed branched, I-type, poly-N-acetyllactosamines with two terminal Galβ4-residues from glycolipids of human stem cells. The structures correlate with expression of β6GlcNAc-transferases capable of branching poly-N-acetyllactosamines and further to binding of lectins specific for branched poly-N-acetylalctosamines. It was further noticed that PWA-lectin had an activity in manipulation of stem cells, especially the growth rate thereof.

Analysis and Utilization of poly-N-acetyllactosamine Sequences and Non-Reducing Terminal Epitopes Associated with Different Glycan Types

The present invention is directed to poly-N-acetyllactosamine sequences (poly-LacNAc) associated with cell types according to the present invention. The inventors found that different types of poly-LacNAc are characteristic to different cell types, as described in the Examples of the present invention. hESC are characterized by type 1 terminating poly-LacNAc, especially on O-glycans and glycolipids. The present invention is especially directed to the analysis and utilization of these glycan characteristics according to the present invention. The present invention is further directed to the analysis and utilization of the specific cell-type accociated glycan sequences revealed in the present Examples according to the present invention.

The present invention is directed to non-reducing terminal epitopes in different glycan classes including N- and O-glycans, glycosphingolipid glycans, and poly-LacNAc. The inventors found that especially the relative amounts of β1,4-linked Gal, β1,3-linked Gal, α1,2-linked Fuc, α1,3/4-linked Fuc, α-linked sialic acid, and α2,3-linked sialic acid are characteristically different between the studied cell types; and the invention is especially directed to the analysis and utilization of these glycan characteristics according to the present invention.

The present invention is further directed to analyzing fucosylation degree in O-glycans by comparing indicative glycan signals such as neutral O-glycan signals at m/z 771 and 917 as described in the Examples. The inventors found that compared to other cell types analyzed in the present invention, hESC had low relative abundance of neutral O-glycan signal at m/z 917 compared to 771, indicating low fucosylation degree of the O-glycan sequences corresponding to the signal at m/z 771 and containing terminal β1,4-linked Gal. Another difference was the occurrence of abundant signal at m/z 552 in hESC, corresponding to Hex₁HexNAc₁dHex₁, including α1,2-fucosylated Core 1 O-glycan sequence. In contrast, in CB MNC the glycan signal at m/z 917 is relatively abundant, indicating high fucosylation degree of the O-glycan sequences corresponding to the signal at m/z 771 and containing terminal β1,4-linked Gal. The other cell types analyzed in the present invention also had characteristic fucosylation degree between these two cell types.

Especially, the present invention is directed to analyzing terminal epitopes associated with poly-LacNAc in stem cells, more preferably when these epitopes are presented in the context of a poly-LacNAc chain, most preferably in O-glycans or glycosphingolipids. The present invention is further directed to analyzing such characteristic poly-LacNAc, terminal epitope, and fucosylation profiles according to the methods of the present invention, in glycan structural characterization and specific glycosylation type identification, and other uses of the present invention; especially when this analysis is done based on endo-β-galactosidase digestion, by studying the non-reducing terminal fragments and their profile, and/or by studying the reducing terminal fragments and their profile, as described in the Examples of the present invention. The inventors found that cell-type specific glycosylation features are efficiently reflected in the endo-β-galactosidase reaction products and their profiles. The present invention is further directed to such reaction product profiles and their analysis according to the present invention.

Especially in hESC, the inventors found that characteristic non-reducing poly-LacNAc associated sequences include Fucα2Gal, Galβ3GlcNAc, Fucα2Galβ3GlcNAc, and α3′-sialylated Galβ3GlcNAc. The present invention is especially directed to analysis of such glycan structures according to the present methods, in context of stem cells and differentiation of stem cells, preferably in context of human embryonic stem cells and their differentiation.

The inventors further found that all three most thoroughly analyzed cellular glycan classes, N-glycans, O-glycans, and glycosphingolipid glycans, were differently regulated compared to each other, especially with regard to non-reducing terminal glycan epitopes and poly-LacNAc sequences as described in the Examples and Tables of the present invention. Therefore, combining quantitative glycan profile analysis data from more than one glycan class will yield significantly more information. The present invention is especially directed to combining glycan data obtained by the methods of the present invention, from more than one glycan class selected from the group of N-glycans, O-glycans, and glycosphingolipid glycans; more preferably, all three classes are analyzed; and use of this information according to the present invention. In a preferred embodiment, N-glycan data is combined with O-glycan data; and in a further preferred embodiment, N-glycan data is combined with glycosphingolipid glycan data.

Lactosamines Galβ3/4GlcNAc and Glycolipid Structures Comprising Lactose Structures (Galβ4Glc)

The lactosamines form a preferred structure group with lactose-based glycolipids. The structures share similar features as products of β3/4Gal-transferases. The β3/4 galactose based structures were observed to produce characteristic features of protein linked and glycolipid glycomes.

The invention revealed that furthermore Galβ3/4GlcNAc-structures are a key feature of differentiation related structures on glycolipids of various stem cell types. Such glycolipids comprise two preferred structural epitopes according to the invention. The most preferred glycolipid types include thus lactosylceramide based glycosphingolipids and especially lacto-(Galβ3GlcNAc), such as lactotetraosylceramide Galβ3GlcNAcβ3Galβ4GlcβCer, preferred structures further including its non-reducing terminal structures selected from the group: Galβ3(Fucα4)GlcNAc (Lewis a), Fucα2Galβ3GlcNAc (H-type 1), structure and, Fucα2Galβ3(Fucα4)GlcNAc (Lewis b) or sialylated structure SAα3Galβ3GlcNAc or SAα3Galβ3(Fucα4)GlcNAc, wherein SA is a sialic acid, preferably Neu5Ac preferably replacing Galβ3GlcNAc of lactotetraosylceramide and its fucosylated and/or elogated variants such as preferably according to the Formula:

(Sacα3)ₙ₅(Fucα2)ₙ₁Galβ3(Fucα4)ₙ₃GlcNAcβ3[Galβ3/4(Fucα4/3)ₙ₂GlcNAcβ3]ₙ₄Galβ4GlcβCer

wherein

n1 is 0 or 1, indicating presence or absence of Fucα2;

n2 is 0 or 1, indicating the presence or absence of Fucα4/3 (branch),

n3 is 0 or 1, indicating the presence or absence of Fucα4 (branch)

n4 is 0 or 1, indicating the presence or absence of (fucosylated) N-acetyllactosamine elongation;

n5 is 0 or 1, indicating the presence or absence of Sacα3 elongation;

Sac is terminal structure, preferably sialic acid, with α3-linkage, with the proviso that when Sac is present, n5 is 1, then n1 is 0 and

neolacto (Galβ4GlcNAc)-comprising glycolipids such as neolactotetraosylceramide Galβ4GlcNAcβ3Galβ4GlcβCer, preferred structures further including its non-reducing terminal Galβ4(Fucα3)GlcNAc (Lewis x), Fucα2Galβ4GlcNAc H-type 2, structure and, Fucα2Galβ4(Fucα3)GlcNAc (Lewis y) and

its fucosylated and/or elogated variants such as preferably (Sacα3/6)ₙ₅(Fucα2)ₙ₁Galβ4(Fucα3)ₙ₃GlcNAcβ3[Galβ4(Fucα3)ₙ₂GlcNAcβ3]ₙ₄Galβ4GlcβCer

n1 is 0 or 1 indicating presence or absence of Fucα2;

n2 is 0 or 1, indicating the presence or absence of Fucα3 (branch),

n3 is 0 or 1, indicating the presence or absence of Fucα3 (branch)

n4 is 0 or 1, indicating the presence or absence of (fucosylated) N-acetyllactosamine elongation,

n5 is 0 or 1, indicating the presence or absence of Sacα3/6 elongation;

Sac is terminal structure, preferably sialic acid (SA) with α3-linkage, or sialic acid with α6-linkage, with the proviso that when Sac is present, n5 is 1, then n1 is 0, and when sialic acid is bound by α6-linkage preferably also n3 is 0.

Preferred Stem Cell Glycosphingolipid Glycan Profiles, Compositions, and Marker Structures

The inventors were able to describe stem cell glycolipid glycomes by mass spectrometric profiling of liberated free glycans, revealing about 80 glycan signals from different stem cell types. The proposed monosaccharide compositions of the neutral glycans were composed of 2-7 Hex, 0-5 HexNAc, and 0-4 dHex. The proposed monosaccharide compositions of the acidic glycan signals were composed of 0-2 NeuAc, 2-9 Hex, 0-6 HexNAc, 0-3 dHex, and/or 0-1 sulphate or phosphate esters. The present invention is especially directed to analysis and targeting of such stem cell glycan profiles and/or structures for the uses described in the present invention with respect to stem cells.

The present invention is further specifically directed to glycosphingolipid glycan signals specific to stem cell types as described in the Examples. In a preferred embodiment, glycan signals typical to hESC, preferentially including 876 and 892 are used in their analysis, more preferentially FucHexHexNAcLac, wherein α1,2-Fuc is preferential to α1,3/4-Fuc, and Hex₂HexNAc₁Lac, and more preferentially to Galβ3[Hex₁HexNAc₁]Lac.

Terminal glycan epitopes that were demonstrated in the present experiments in stem cell glycosphingolipid glycans are useful in recognizing stem cells or specifically binding to the stem cells via glycans, and other uses according to the present invention, including terminal epitopes: Gal, Galβ4Glc (Lac), Galβ4GlcNAc (LacNAc type 2), Galβ3, Non-reducing terminal HexNAc, Fuc, α1,2-Fuc, α1,3-Fuc, Fucα2Gal, Fucα2Galβ4GlcNAc (H type 2), Fucα2Galβ4Glc (2′-fucosyllactose), Fucα3GlcNAc, Galβ4(Fucα3)GlcNAc (Lex), Fucα3Glc, Galβ4(Fucα3)Glc (3-fucosyllactose), Neu5Ac, Neu5Acα2,3, and Neu5Acα2,6. The present invention is further directed to the total terminal epitope profiles within the total stem cell glycosphingolipid glycomes and/or glycomes.

The inventors were further able to characterize in hESC the corresponding glycan signals to SSEA-3 and SSEA-4 developmental related antigens, as well as their molar proportions within the stem cell glycome. The invention is further directed to quantitative analysis of such stem cell epitopes within the total glycomes or subglycomes, which is useful as a more efficient alternative with respect to antibodies that recognize only surface antigens. In a further embodiment, the present invention is directed to finding and characterizing the expression of cryptic developmental and/or stem cell antigens within the total glycome profiles by studying total glycan profiles, as demonstrated in the Examples for α1,2-fucosylated antigen expression in hESC in contrast to SSEA-1 expression in mouse ES cells.

The present invention revealed characteristic variations (increased or decreased expression in comparision to similar control cell or a contaminating cell or like) of both structure types in various cell materials according to the invention. The structures were revealed with characteristic and varying expression in three different glycome types: N-glycans, O-glycans, and glycolipids. The invention revealed that the glycan structures are a characteristic feature of stem cells and are useful for various analysis methods according to the invention. Amounts of these and relative amounts of the epitopes and/or derivatives varies between cell lines or between cells exposed to different conditions during growing, storage, or induction with effector molecules such as cytokines and/or hormones.

Preferred Epitopes and Antibody Binders especially for Analysis of Embryonic Stem Cells

The antibody labelling experiment Table 48 with embryonic stem cells revealed specific of type 1 N-acetyllactosamine antigen recognizing antibodies recognizing non-modified disaccharide Galβ3GlcNAc (Le c, Lewis c), and fucosylated derivatives H type and Lewis b. The antibodies were effective in recognizing hESC cell populations in comparision to mouse feeder cells mEF used for cultivation of the stem cells. See Figures for results.

Specific different H type 2 recognizing antibodies were revealed to recognize different subpopulations of embryonic stem cells and thus usefulness for defining subpopulations of the cells. The invention further revealed a specific Lewis x and sialyl-Lewis x structures on the embryonic stem cells.

Other preferred binders and/or antibodies comprise of binders which bind to the same epitope than GF 287 (H type 1). In a preferred embodiment, an antibody binds to Fucα2Galβ3GlcNAc epitope. A more preferred antibody comprises of the antibody of clone 17-206 (ab3355) by Abcam. This epitope is suitable and can be used to detect, isolate and evaluate the differentiation stage, and/or plucipotency of stem cells, preferably human embryonic stem cells. The detection can be performed in vitro, for FACS purposes and/or for cell lineage specific purposes. This antibody can be used to positively isolate and/or separate and/or enrich stem cells, preferably human embryonic stem cells from a mixture of cells comprising feeder and stem cells.

Other preferred binders and/or antibodies comprise of binders which bind to the same epitope than GF 279 (Lewis c, Galβ3GlcNAc). In a preferred embodiment, an antibody binds to Galβ3GlcNAc epitope in glycoconjugates, more preferably in glycoproteins and glycolipids such as lactotetraosylceramide. A more preferred antibody comprises of the antibody of clone K21 (ab3352) by Abcam. This epitope is suitable and can be used to detect, isolate and evaluate the differentiation stage, and/or plucipotency of stem cells, preferably human embryonic stem cells. The detection can be performed in vitro, for FACS purposes and/or for cell lineage specific purposes. This antibody can be used to positively isolate and/or separate and/or enrich stem cells, preferably human embryonic stem cells from a mixture of cells comprising feeder and stem cells.

Other preferred binders and/or antibodies comprise of binders which bind to the same epitope than GF 288 (Globo H). In a preferred embodiment, an antibody binds to Fucα2Galβ3GalNAcβ epitope, more preferably Fucα2Galβ3GalNAcβ3GalαLacCer epitope. A more preferred antibody comprises of the antibody of clone A69-A/E8 (MAB-S206) by Glycotope. This epitope is suitable and can be used to detect, isolate and evaluate the differentiation stage, and/or plucipotency of stem cells, preferably human embryonic stem cells. The detection can be performed in vitro, for FACS purposes and/or for cell lineage specific purposes. This antibody can be used to positively isolate and/or separate and/or enrich stem cells, preferably human embryonice stem cells from a mixture of cells comprising feeder and stem cells.

Other preferred binders and/or antibodies comprise of binders which bind to the same epitope than GF 284 (H type 2). In a preferred embodiment, an antibody binds to Fucα2Galβ4GlcNAc epitope. A more preferred antibody comprises of the antibody of clone B393 (DM3015) by Acris. This epitope is suitable and can be used to detect, isolate and evaluate the differentiation stage, and/or plucipotency of stem cells, preferably human embryonic stem cells. The detection can be performed in vitro, for FACS purposes and/or for cell lineage specific purposes. This antibody can be used to positively isolate and/or separate and/or enrich stem cells, preferably human embryonice stem cells from a mixture of cells comprising feeder and stem cells.

Other preferred binders and/or antibodies comprise of binders which bind to the same epitope than GF 283 (Lewis b). In a preferred embodiment, an antibody binds to Fucα2Galβ3(Fucα4)GlcNAc epitope. A more preferred antibody comprises of the antibody of clone 2-25LE (DM3122) by Acris. This epitope is suitable and can be used to detect, isolate and evaluate the differentiation stage, and/or plucipotency of stem cells, preferably human embryonic stem cells. The detection can be performed in vitro, for FACS purposes and/or for cell lineage specific purposes. This antibody can be used to positively isolate and/or separate and/or enrich stem cells, preferably human embryonice stem cells from a mixture of cells comprising feeder and stem cells.

Other preferred binders and/or antibodies comprise of binders which bind to the same epitope than GF 286 (H type 2). In a preferred embodiment, an antibody binds to Fucα2Galβ4GlcNAc epitope. A more preferred antibody comprises of the antibody of clone B393 (BM258P) by Acris. This epitope is suitable and can be used to detect, isolate and evaluate the differentiation stage, and/or plucipotency of stem cells, preferably human embryonic stem cells. The detection can be performed in vitro, for FACS purposes and/or for cell lineage specific purposes. This antibody can be used to positively isolate and/or separate and/or enrich stem cells, preferably human embryonice stem cells from a mixture of cells comprising feeder and stem cells.

Other preferred binders and/or antibodies comprise of binders which bind to the same epitope than GF 290 (H type 2). In a preferred embodiment, an antibody binds to Fucα2Galβ4GlcNAc epitope. A more preferred antibody comprises of the antibody of clone A51-B/A6 (MAB-S204) by Glycotope. This epitope is suitable and can be used to detect, isolate and evaluate the differentiation stage, and/or plucipotency of stem cells, preferably human embryonic stem cells. The detection can be performed in vitro, for FACS purposes and/or for cell lineage specific purposes. This antibody can be used to positively isolate and/or separate and/or enrich stem cells, preferably human embryonice stem cells from a mixture of cells comprising feeder and stem cells.

Other binders binding to feeder cells, preferably mouse feeder cells, comprise of binders which bind to the same epitope than GF 285 (H type 2). In a preferred embodiment, an antibody binds to Fucα2Galβ4GlcNAc, Fucα2Galβ3(Fucα4)GlcNAc, Fucα2Galβ4(Fucα3)GlcNAc epitope. A more preferred antibody comprises of the antibody of clone B389 (DM3014) by Acris. This epitope is suitable and can be used to detect, isolate and evaluate of feeder cells, preferably mouse feeder cells in culture with human embryonic stem cells. The detection can be performed in vitro, for FACS purposes and/or for cell lineage specific purposes. This antibody can be used to positively isolate and/or separate and/or enrich feeder cells (negatively select stem cells), preferably mouse embryonic feeder cells from a mixture of cells comprising feeder and stem cells.

Other binders binding to stem cells, preferably human stem cells, comprise of binders which bind to the same epitope than GF 289 (Lewis y). In a preferred embodiment, an antibody binds to Fucα2Galβ4(Fucα3)GlcNAc epitope. A more preferred antibody comprises of the antibody of clone A70-C/C8 (MAB-S201) by Glycotope. This epitope is suitable and can be used to detect, isolate and evaluate of stem cells, preferably human stem cells in culture with feeder cells. The detection can be performed in vitro, for FACS purposes and/or for cell lineage specific purposes. This antibody can be used to positively isolate and/or separate and/or enrich stem cells (negatively select feeder cells), preferably human stem cells from a mixture of cells comprising feeder and stem cells.

The staining intensity and cell number of stained stem cells, i.e. glycan structures of the present invention on stem cells indicates suitability and usefulness of the binder for isolation and differentiation marker. For example, low relative number of a glycan structure expressing cells may indicate lineage specificity and usefulness for selection of a subset and when selected/isolated from the colonies and cultured. Low number of expression is less than 5%, less than 10%, less than 15%, less than 20%, less than 30% or less than 40%. Further, low number of expression is contemplated when the expression levels are between 1-10%, 10%-20%, 15-25%, 20-40%, 25-35% or 35-50%. Typically, FACS analysis can be performed to enrich, isolate and/or select subsets of cells expressing a glycan structure(s).

High number of glycan expressing cells may indicate usefulness in pluripotency/multipotency marker and that the binder is useful in identifying, characterizing, selecting or isolating pluripotent or multipotent stem cells in a population of mammalian cells. High number of expression is more than 50%, more preferably more than 60%, even more preferably more than 70%, and most preferably more than 80%, 90 or 95%. Further, high number of expression is contemplated when the expression levels are between 50-60, 55%-65%, 60-70%, 70-80, 80-90%, 90-100 or 95-100%. Typically, FACS analysis can be performed to enrich, isolate and/or select subsets of cells expressing a glycan structure(s).

The epitopes recognized by the binders GF 279, GF 287, and GF 289 and the binders are particularly useful in characterizing pluripotency and multipotency of stem cells in a culture. The epitopes recognized by the binders GF 283, GF 284, GF 286, GF 288, and GF 290 and the binders are particularly useful for selecting or isolating subsets of stem cells. These subset or subpopulations can be further propagated and studied in vitro for their potency to differentiate and for differentiated cells or cell committed to a certain differentiation path.

The percentage as used herein means ratio of how many cells express a glycan structure to all the cells subjected to an analysis or an experiment. For example, 20% stem cells expressing a glycan structure in a stem cell colony means that a binder, eg an antibody staining can be observed in about 20% of cells when assessed visually.

In colonies a glycan structure bearing cells can be distributed in a particular regions or they can be scattered in small patch like colonies. Patch like observed stem cells are useful for cell lineage specific studies, isolation and separation. Patch like characteristics were observed with GF 283, GF 284, GF 286, GF 288, and GF 290.

For positive selection of feeder cells, preferably mouse feeder cells, most preferably embryonic fibroblasts, GF 285 is useful. This antibody has lower specificity and may have binding to e.g. Lewis y, which has been observed also in mEF cells. It stains almost all feeder cells whereas very little if at all staining is found in stem cells. The antibody was however under optimized condition revealed to bind to thin surface of embryonic bodies, this was in complementary to Lewis y antibody to the core of embryoid body. For all percentages of expression in immunohistochemical analysis, see Table 48.

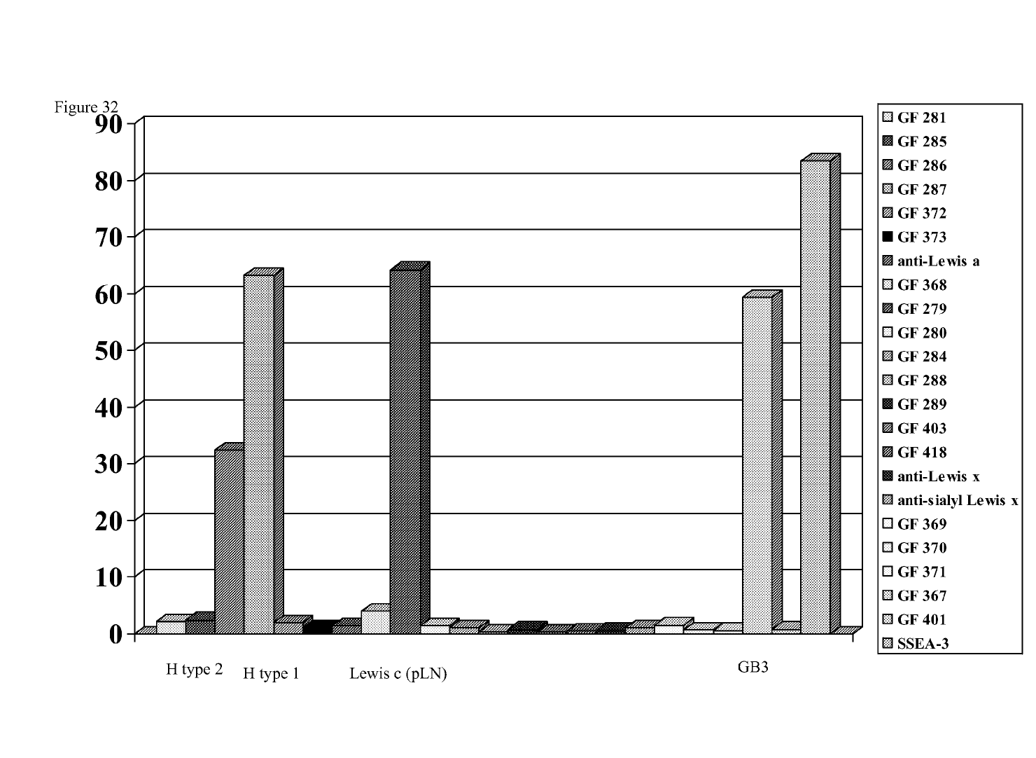

The FACS data in Tables 18, 46-47 and FIG. 32 indicates some antibodies recognizing the major elongated glycan structure epitopes according to the invention on cell surfaces. The invention is especially directed to the use of the H type II, H type I, type I LacNAc (Lewis c) and globotriose specific antibodies for the recognition of the embryonic stem cells, GF286, GF287, GF 279 and GF367. The invention is further directed to the major cell populations isolatable by the antibodies. The invention is further directed to the antibodies with similar specificities as the antibodies recognizing the major cell population of the embryonal stem cells. The invention is preferably directed to recognition of the elongated epitopes of H type II and H type I and type I LacNAc structures according to the invention by specific binder regents, preferably by antibodies. The invention is further directed to the recognition of the novel stem cell marker globotriose from the embryonal type stem cells and isolation of the cell population by the by using the specific binder for the glycan structure.

The invention is in a preferred embodiment directed to the short globoseries structures such as globotriose non-reducing end globotriose (Gb3) epitopes: Galα4Gal, Galα4Galβ and Galα4Galβ4Glc for the methods according to the invention. In a preferred embodiment the invention is directed to the recognition of the ceramide linked globotriose epitope. It is realized that though larger globoseries structures SSEA-3 and SSEA-4 has been indicated from embryonic stem cells, this structure has not been known from embryonic type stem cells and their amounts have been unpredictable.

Novel Methods for Recognition of hESC Differentiation Stage Derived from the Factor Analyses

Here, statistical analysis was used to identify indicative glycan signals, glycan structures, and glycan structure groups for specific recognition of hESC and differentiated cells. The inventors revealed that by factor analysis several differentially regulated glycan groups could be identified among the N-glycan profiles of hESC and differentiated cells (embryoid bodies and stage 3 differentiated cells). According to the invention, the cell's differentiation stage can be assessed by both positively and negatively selective glycan structures and glycan structure groups, preferably by those described above. Specifically, the factor analysis revealed novel advantageous combinations of positively+positively, positively+negatively, and negatively+negatively selective glycan structures for recognition of the differentiation stage of hESC.

The present invention is specifically directed to performing such analysis by direct analysis of the glycan profiles of hESC and differentiated cells, preferably by mass spectrometry according to the present invention, the novel added benefit being more effective and reliable interpretation of the analysis result.

In a further embodiment of the present invention, cells in a specific differentiation stage are recognized by a glycan structure specific binding reagent, and further specificity can be gained by selecting the reagent according to the revealed cell type specificities of the recognized glycan groups. The present invention is specifically directed to selected binding reagents according to the invention, when the selection is guided by the analysis results described above. The invention is further specifically directed to using combinations of binding reagents selected based on selectivity of glycan structures revealed in the present invention.

In a further embodiment, the positively and negatively selective binding reagents are selected based on the Tables 50 and 51, respectively.

For example, novel beneficial combinations for recognition of hESC differentiation stage is selection of at least two specific binding reagents recognizing glycan structures in at least two different glycan structure groups of Tables 50 and 51. An even more beneficial combination for specific recognition is selection of at least two specific binding reagents recognizing glycan structures, at least one in each Table.

The binding reagents selected specifically recognizes at least one preferred elongated glycan epitopes according to the invention. More preferably preferred elongated N-glycan epitopes, preferably β2Man-epitopes, even more preferably elongated type II LacNAc, sialylated and fucosylated derivatives thereof including Lewis x, H type II, and sialyl-Lewis x. The invention is further directed to reagents recognizing terminal mannose epitopes of the high and low mannose glycans identified.

### Examples

### Example 1

### Analysis of the Human Embryonic Stem Cell N-Glycome

Structural proposals for N-glycan signals characterized by m/z values as the other Tables of the present invention, is presented in Tables 12 and 13. The N-glycan schematic structures are according to the recommendations of the Consortium for Functional Glycomics (www.functionalglycomics.org) and as described e.g. in Goldberg et al. (2005) Proteomics 5, 865-875.

Materials and Methods

Human embryonic stem cell lines (hESC)—Generation of the Finnish hESC lines FES 21, FES 22, FES 29, and FES 30 has been described (17) and they were cultured according to the previous report. Briefly, two of the analysed cell lines were initially derived and cultured on mouse embryonic fibroblast (MEF) feeders, and two on human foreskin fibroblast (HFF) feeder cells. For the present studies all of the lines were transferred on HFF feeder cells and cultured in serum-free medium supplemented with Knockout serum replacement (Gibco). To induce the formation of embryoid bodies (EB) the hESC colonies were first allowed to grow for 10-14 days whereafter the colonies were cut in small pieces and transferred on non-adherent Petri dishes to form suspension cultures. The formed EBs were cultured in suspension for the next 10 days in standard culture medium without bFGF. For further differentiation (into stage 3 differentiated cells) EB were transferred onto gelatin-coated culture dishes in media supplemented with insulin-transferrin-selenium and cultured for 10 days.

For glycan analysis, the cells were collected mechanically, washed, and stored frozen until the analysis. In fluorescence-assisted cell sorting (FACS) analyses 70-90% of cells from mechanically isolated hESC colonies were typically Tra 1-60 and Tra 1-81 positive (not shown). The differentiation protocol favors the development of neuroepithelial cells while not directing the differentiation into distinct terminally differentiated cell types (18). Stage 3 cultures consisted of a heterogenous population of cells dominated by fibroblastoid and neuronal morphologies.

Glycan isolation—Asparagine-linked glycans were detached from cellular glycoproteins by F. meningosepticum N-glycosidase F digestion (Calbiochem, USA) essentially as described (19). Cellular contaminations were removed by precipitating the glycans with 80-90% (v/v) aqueous acetone at −20° C. and extracting them with 60% (v/v) ice-cold methanol (20). The glycans were then passed in water through C₁₈ silica resin (BondElut, Varian, USA) and adsorbed to porous graphitized carbon (Carbograph, Alltech, USA) (21). The carbon column was washed with water, then the neutral glycans were eluted with 25% acetonitrile in water (v/v) and the sialylated glycans with 0.05% (v/v) trifluoroacetic acid in 25% acetonitrile in water (v/v). Both glycan fractions were additionally passed in water through strong cation-exchange resin (Bio-Rad, USA) and C₁₈ silica resin (ZipTip, Millipore, USA). The sialylated glycans were further purified by adsorbing them to microcrystalline cellulose in n-butanol:ethanol:water (10:1:2, v/v), washing with the same solvent, and eluting by 50% ethanol:water (v/v). All the above steps were performed on miniaturized chromatography columns and small elution and handling volumes were used.

Mass spectrometry and data analysis—MALDI-TOF mass spectrometry was performed with a Bruker Ultraflex TOF/TOF instrument (Bruker, Germany) essentially as described (22). Relative molar abundancies of neutral and sialylated glycan components can be accurately assigned based on their relative signal intensities in the mass spectra when analyzed separately as the neutral and sialylated N-glycan fractions (22-25). Each step of the mass spectrometric analysis methods was controlled for reproducibility by mixtures of synthetic glycans or glycan mixtures extracted from human cells.

The mass spectrometric raw data was transformed into the present glycan profiles by carefully removing the effect of isotopic pattern overlapping, multiple alkali metal adduct signals, products of elimination of water from the reducing oligosaccharides, and other interfering mass spectrometric signals not arising from the original glycans in the sample. The resulting glycan signals in the presented glycan profiles were normalized to 100% to allow comparison between samples.

Quantitative difference between two glycan profiles (%) was calculated according to Equation 1:

$$difference = \frac{1}{2} {\sum }_{i = 1}^{n} \vert {p}_{i , a} - {p}_{i , b} \vert , \qquad ( 1 )$$

wherein p is the relative abundance (%) of glycan signal i in profile a or b, and n is the total number of glycan signals.

Relative difference between a glycan feature in two profiles was calculated according to Equation 2:

$$relative difference = {x ( \frac{{P}_{a}}{{P}_{b}} )}^{x} , \qquad ( 2 )$$

wherein P is the sum the relative abundancies of the glycan signals with the glycan feature in profile a or b, x is 1 when a≧b, and x is −1 when a<b.

The glycan analysis method was validated by subjecting human cell samples to blinded analysis by five different persons. The results were highly comparable (data not shown), especially by the terms of detection of individual glycan signals and their relative signal intensities, showing that the present method reliably produced glycan profiles suitable for comparision of analysis results from different cell types.

Glycosidase analysis—The neutral N-glycan fraction was subjected to digestion with Jack bean α-mannosidase (Canavalia ensiform is; Sigma, USA) essentially as described (22).

NMR methods—For NMR spectroscopic analyses, larger amounts of hESC were grown on mouse feeder cell (MEF) layers. The isolated glycans were purified for the analysis by gel filtration high-pressure liquid chromatography in a column of Superdex peptide HR 10/30 (Amersham), with water (neutral glycans) or 50 mM NH₄HCO₃ (sialylated glycans) as the eluant at a flow rate of 1 ml/min. The eluant was monitored at 214 nm, and oligosaccharides were quantified against external standards. The amount of N-glycans in NMR analysis was below five nanomoles. Prior to NMR analysis the purified glycome fractions were repeatedly dissolved in 99.996% deuterium oxide and dried to omit H₂O and to exchange sample protons. The proton NMR spectra at 800 MHz were recorded using a cryo-probe for enhanced sensitivity.

Statistical procedures—Glycan score distributions of all three differentiation stages (hESC, EB, and stage 3 differentiated cells) were analyzed by the Kruskal-Wallis test. Pairwise comparisons were performed by the 2-tailed Student's t-test with Welch's approximation and 2-tailed Mann-Whitney U test. A p value less than 0.05 was considered significant. The statistical analyses are described in more detail in Supplementary data.

Lectin staining—Fluorescein-labelled lectins used in lectin histochemistry were from EY Laboratories (USA). Specificity of binding was controlled by inhibition experiments with α3′-sialyllactose and D-mannose for Maackia amurensis agglutinin (MAA) and Pisum sativum agglutinin (PSA), respectively.

Results

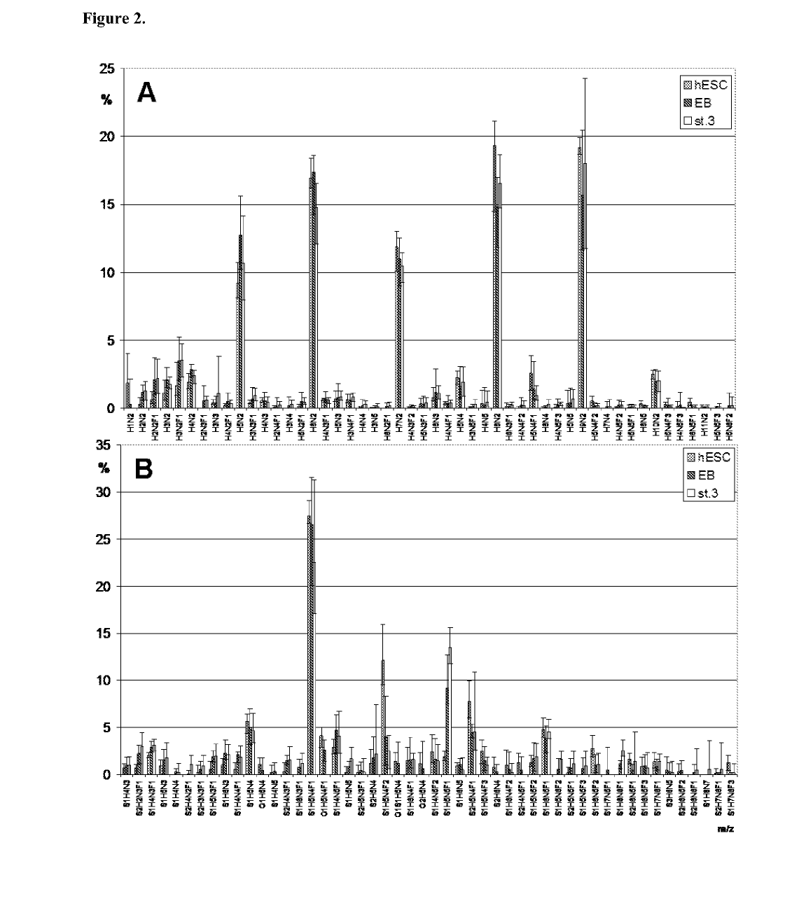

In order to generate mass spectrometric glycan profiles of hESC, embryoid bodies (EB), and further differentiated cells, a matrix-assisted laser desorption-ionization (MALDI-TOF) mass spectrometry based analysis was performed. We focused on the most common type of protein post-translational modifications, N-glycans, which were enzymatically released from cellular glycoproteins. During glycan isolation and purification, the total N-glycan pool was separated by an ion-exchange step into neutral N-glycans and sialylated N-glycans. These two glycan fractions were then analyzed separately by mass spectrometric profiling (FIG. 2), which yielded a global view of the N-glycan repertoire. Over one hundred N-glycan signals were detected from each cell type demonstrating that N-glycosylation is equally sophisticated in stem cells and cells differentiated from them. The proposed monosaccharide compositions corresponding to the detected masses of each individual signal in FIG. 2 are indicated by letter code. However, it is important to realize that many of the mass spectrometric signals in the present analyses include multiple isomeric structures and the one hundred most abundant signals very likely represent hundreds of different molecules.

The relative abundances of the observed glycan signals were determined based on their relative signal intensities (22,24-25), which allowed analysis of N-glycan profile differences between samples. The present data demonstrate that mass spectrometric profiling can be used in effective quantitative comparison of total glycan profiles, especially to pin-point the major glycosylation differences between related samples. In the following, we have expressed relative abundancies of glycan signals as molar proportions of the total detected N-glycans. However, these figures should be recognized as practical approximations based on the present data instead of absolutely quantitative percentages of the N-glycome.

In most of the previous glycomic studies of mammalian cells and tissues the isolated glycans have been derivatized (permethylated) prior to mass spectrometric profiling (26-29) or chromatographic analysis (30).

However, we chose to directly analyze the picomolar quantities of unmodified glycans and increased sensitivity was achieved by omitting the derivatization and the subsequent additional purification steps. Our glycan purification scheme enabled N-glycan profiling analysis from samples as small as 100 000 cells showing that sensitivity of the analysis step is not a limiting factor in glycomic studies with scarce biological samples.

Overview of the hESC N-glycome: Neutral N-glycans Neutral N-glycans comprised approximately two thirds of the combined neutral and sialylated N-glycan pools of hESC. The 50 most abundant neutral N-glycan signals detected in the four hESC lines are presented in FIG. 2A (blue columns). The similarity of the profiles, which is indicated by the minor variation in the glycan signals, suggests that the four cell lines closely resemble each other. For example, 15 of the 20 most abundant glycan signals were the same in every hESC line. These 15 neutral N-glycan signals characteristic of the hESC N-glycome are listed in Table 7. The five most abundant signals (II₅N₂, II₆N₂, II₇N₂, II₈N₂, and II₉N₂; for abbreviations see FIG. 2) comprised 76% of the neutral N-glycans of hESC and dominated the profile.

Sialylated N-glycans—All N-glycan signals in the sialylated N-glycan fraction (FIG. 2B, blue columns) contained sialic acid residues (S: N-acetylneuraminic acid, or G: N-glycolylneuraminic acid). There was more variation between individual cell lines in the 50 most abundant sialylated N-glycans than in the neutral N-glycans. However, the four cell lines again resembled each other. The five most abundant sialylated N-glycan signals were the same in every cell line: S₁H₅N₄F₁, S₁H₅N₄F₂, S₂H₅N₄F₁, S₁H₅N₄, and S₁H₆N₅F₁. The 15 sialylated N-glycan signals common to all the hESC lines are listed in Table 7.

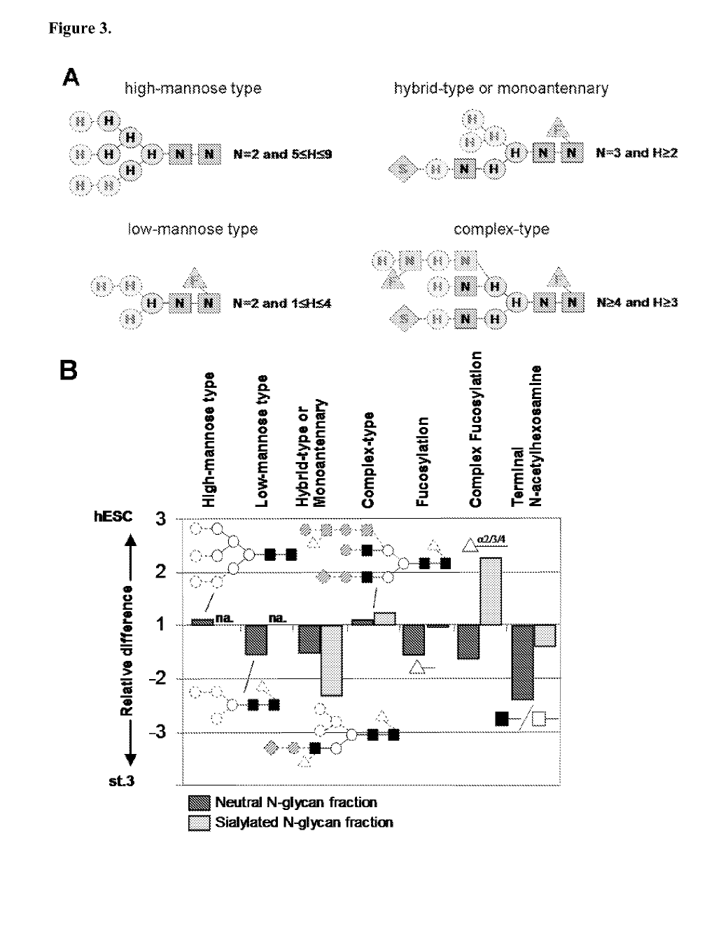

The most abundant sialylated glycan signals contained the H₅N₄ core composition and differed only by variable number of sialic acid (S or G) and deoxyhexose (F) residues. These comprised 61% of the total glycan signal intensity in FIG. 2B. Similarly, another common core structure was H₆N₅ that was present in seven signals comprising 12% of the total glycan signal intensity. These examples highlight the biosynthetic mechanism that leads to the complex spectra of N-glycan structures in cells: N-glycans typically consist of common core structures that are modified by the addition of variable epitopes (FIG. 3A).

Importantly, we detected N-glycans containing N-glycolylneuraminic acid (G) in the hESC samples, for example glycans G₁H₅N₄, G₁S₁H₅N₄, and G₂H₅N₄. N-glycolylneuraminic acid has previously been reported in hESC as an antigen transferred from culture media containing animal-derived materials (31). Accordingly, the serum replacement medium used in the present experiments contained bovine serum proteins. We have recently detected Neu5Gc in N-glycans of hESC and in vitro cultured human mesenchymal stem cells by mass spectrometric N-glycan analysis (32).

Variation between individual cell lines—Although the four hESC lines shared the same overall N-glycan profile, there was cell line specific variation within the profiles. Individual glycan signals unique to each cell line were detected, indicating that every cell line was slightly different from each other with respect to the approximately one hundred most abundant N-glycan structures. Importantly, the 30 most common N-glycan signals in all the hESC lines accounted for circa 85% of the total detected N-glycans, and they represent a useful approximation of the hESC N-glycome (Table 7).

Transformation of the N-glycome during hESC differentiation—A major goal of the present study was to identify glycan structures that would be specific to either stem cells or differentiated cells, and could therefore serve as differentiation stage markers. In order to determine whether the hESC N-glycome undergoes changes during differentiation, the N-glycan profiles obtained from hESC, EB, and stage 3 differentiated cells were compared (FIG. 2). The profiles of the differentiated cell types (EB and stage 3 differentiated cells) were clearly different compared to the profiles of undifferentiated hESC, as indicated by non-overlapping distribution bars in many glycan signals. Further, there were many signals present in both hESC and EB that were not detected in stage 3 differentiated cells. Overall, 10% of the glycan signals present in hESC had disappeared in stage 3 differentiated cells. Simultaneously numerous new signals appeared in EB and stage 3 differentiated cells. The proportion of these differentiation-associated N-glycan signals in EB and stage 3 differentiated cells was 14% and 16%, respectively.

Taken together, differentiation induced the appearance of new N-glycan types while earlier glycan types disappeared. Further, we found that the major hESC-specific N-glycosylation features were not expressed as discrete glycan signals, but instead as glycan signal groups that were characterized by specific monosaccharide composition features. In other words, differentiation of hESC into EB induced the disappearance of not only one but multiple glycan signals with hESC-associated features, and simultaneously also the appearance of glycan signal groups with other, differentiation-associated features.

The N-glycan profiles of the differentiated cells were also quantitatively different from the undifferentiated hESC profiles. A practical way of quantifying the differences between glycan profiles is to calculate the sum of the signal intensity differences between two samples (see Experimental procedures, Equation 1). According to this method, the EB neutral and sialylated N-glycan profiles had undergone a quantitative change of 14% and 29% from the hESC profiles, respectively. Similarly, the stage 3 differentiated cell neutral and sialylated N-glycan profiles had changed by 15% and 43%, respectively. Taking into account that the proportion of sialylated to neutral N-glycans in hESC was approximately 1:2, the total N-glycan profile change was approximately 25% during the transition from hESC to stage 3 differentiated cells.

The present data indicated that the mass spectrometric profile of the hESC N-glycome consisted of two discrete parts regarding propensity to change during hESC differentiation—a constant part of circa 75% and a changing part of circa 25%. In order to characterize the associated N-glycan structures, and to identify the potential biological roles of the constant and changing parts of the N-glycome, we performed structural analyses of the isolated hESC N-glycan samples.

Structural analyses of the major hESC N-glycans: Preliminary structure assignment based on monosaccharide compositions—Human N-glycans can be divided into biosynthetic groups of high-mannose type, hybrid-type, and complex-type N-glycans (33-34). Due to abundant expression of mannosylated N-glycans smaller than the classical high-mannose type structures in hESC, we added a new group called low-mannose N-glycans into this classification. To determine the presence of these N-glycan groups in the cells, assignment of probable structures matching the monosaccharide compositions of each individual signal was performed utilizing the established pathways of human N-glycan biosynthesis. Here, the detected N-glycan signals were classified into four N-glycan groups according to the number of N and H residues in the proposed compositions as shown in FIG. 3A: 1) high-mannose type and 2) low-mannose type N-glycans, which are both characterized by two N residues (N−2), 3) hybrid-type or monoantennary N-glycans, which are classified by three N residues (N=3), and 4) complex-type N-glycans, which are characterized by four or more N residues (N≧4) in their proposed monosaccharide compositions. However, this is an approximation and in addition to complex-type N-glycans also hybrid-type or monoantennary N-glycans may contain more than three N residues.

The data was analyzed quantitatively by calculating the percentage of glycan signals in the total N-glycome belonging to each structure group (Table 3) and comparing the hESC and differentiated cell glycan classification data (FIG. 3B). The relative differences in the structural groups reflect the activities of different biosynthetic pathways in each cell type. For example, the proportion of hybrid-type or monoantennary N-glycans was increased when hESC differentiated into EB, indicating that different glycan biosynthesis routes were favored in EB than in hESC. However, no glycan structure classes disappeared or appeared in the hESC differentiation process, which indicated that the fundamental N-glycan biosynthesis routes were not changed during differentiation. The proportion of low-mannose type N-glycans was surprisingly high in the light of earlier published studies of human N-glycosylation. However, according to our studies this is not specific to hESC (T. Satomaa, A. Heiskanen, J. Natunen, J. Saarinen, N. Salovuori, A. Olonen, J. Helin, M. Blomqvist, O. Carpén, unpublished results).

Verification of structure assignments by enzymatic glycan degradation and nuclear magnetic resonance spectroscopy—In order to validate the glycan structure assignments made based on the mass spectrometric analysis and the proposed monosaccharide compositions, we performed enzymatic degradation and proton NMR spectroscopy analyses of selected neutral and sialylated N-glycans.

For the validation of neutral N-glycans we chose the glycans H₅N₂, H₆N₂, H₇N₂, H₈N₂, which were the most abundant N-glycans in all studied cell types (FIG. 2A). The monosaccharide compositions of these glycans had already suggested (FIG. 3A) that they were high-mannose type N-glycans (33). To test this hypothesis, neutral N-glycans from hESC and the differentiated cell samples were treated with α-mannosidase, and analyzed both before and after the enzymatic treatment by MALDI-TOF mass spectrometry (data not shown). The glycans in question were degraded and the corresponding signals disappeared from the mass spectra, indicating that they had contained α-linked mannose residues.

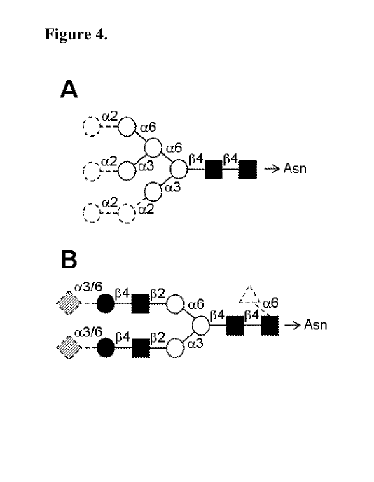

The neutral N-glycan fraction was further analyzed by nanoscale proton NMR spectroscopy. In the obtained NMR spectrum of the hESC neutral N-glycans signals consistent with high-mannose type N-glycans were abundant (FIG. 4A and Table 8), supporting the conclusion that they were the major glycan components in the sample. In proton NMR spectroscopic analysis of the sialylated N-glycan fraction, N-glycan backbone signals consistent with biantennary complex-type N-glycans were the major detected signals FIG. 4B and Table 9), in line with the preliminary assignment made based on the proposed monosaccharide compositions. The present results indicated that the classification of the glycan signals within the total N-glycome data could be used to construct an approximation of the whole N-glycome.

Complex fucosylation of N-glycans is characteristic of hESC—Differentiation stage associated changes in the sialylated N-glycan profile of hESC were more drastic than in the neutral N-glycan fraction and the group of five most abundant sialylated N-glycan signals was different at every differentiation stage (FIG. 2B). In particular, there was a significant differentiation-associated decrease in the relative amounts of glycans S₁H₅N₄F₂ and S₁H₅N₄F₃ as well as other glycan signals that contained at least two deoxyhexose (F≧2). In contrast, glycan signals such as S₂H₅N₄ that contained no F were increased in the differentiated cell types. The results suggested that sialylated N-glycans in undifferentiated hESC were subject to more complex fucosylation than in the differentiated cell types (FIG. 3B). The most common fucosylation type in human N-glycans is α1,6-fucosylation of the N-glycan core structure (35). The NMR analysis of the sialylated N-glycan fraction of hESC also revealed α1,6-fucosylation of the N-glycan core as the most abundant type of fucosylation (Table 9). In N-glycans containing more than one fucose residue there has to be other fucose linkages in addition to the α1,6-linkage (35). The F≧2 structural feature decreased as the cells differentiated, indicating that complex fucosylation was characteristic of undifferentiated hESC.

N-glycans with terminal N-acetylhexosamine residues become more common with differentiation—A major group of N-glycan signals which increased during differentiation contained equal amounts of N-acetylhexosamine and hexose residues (N═H) in their monosaccharide composition (e.g. S₁H₅N₅F₁). This was consistent with N-glycan structures containing non-reducing terminal N-acetylhexosamine residues since such complex-type N-glycans generally have monosaccharide compositions of either N═H or N>H (FIG. 3A). EB and stage 3 differentiated cells showed increased amounts of potential terminal N-acetylhexosamine structures (FIG. 3B).

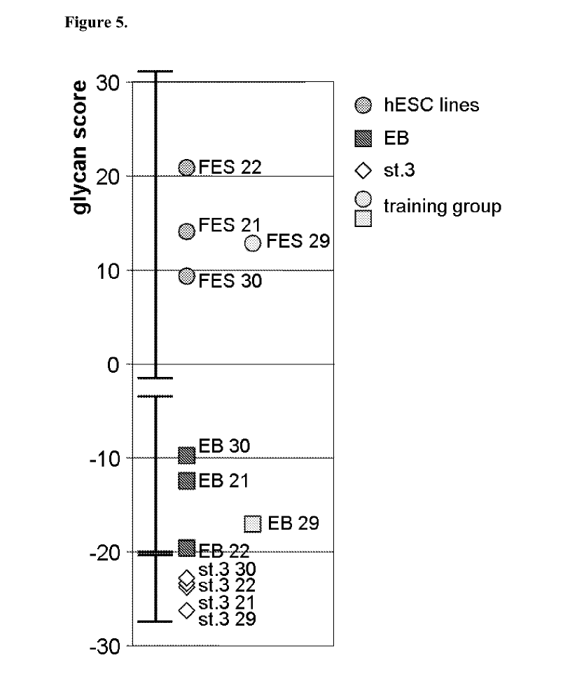

Glycome profiling can identify the differentiation stage of hESC—The glycome profile analyses indicated that the studied hESC lines and differentiated cells had differentiation stage specific N-glycosylation features. However, the data also demonstrated variation between individual cell lines. To test whether the obtained N-glycan profiles could be used to generate an efficient discrimination algorithm that would discriminate between hESC and differentiated cells, we performed a statistical evaluation of the mass spectrometric data (see Supplementary data for details). The results are described graphically in FIG. 5. The differentiated cell samples (EB and stage 3 differentiated cells) were significantly discriminated from hESC with p<0.01. The stage 3 differentiated cell samples were also significantly separated from the EB samples with p<0.01. This suggested that the hESC N-glycan profiles were similar at the glycome level despite of individual differences at the level of individual glycan signals. The result also suggested that glycome profiling is a potential tool for monitoring the differentiation status of stem cells.

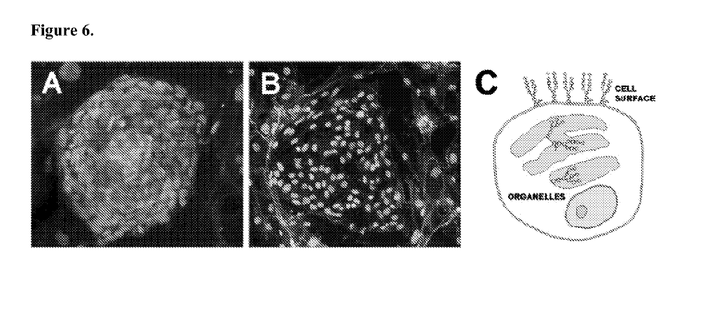

The identified hESC glycans can be targeted at the cell surface—From a practical perspective stem cell research would be best served by reagents that recognize cell-type specific target structures on cell surface. To investigate whether individual glycan structures we had identified would be accessible to reagents targeting them at the cell surface we performed lectin labelling of two candidate structure types. Lectins are proteins that recognize glycans with specificity to certain glycan structures also in hESC (36-37). hESC colonies grown on mouse feeder cell layers were labeled in vitro by fluorescein-labelled lectins (FIG. 6). The hESC cell surfaces were clearly labeled by Maackia amurensis agglutinin (MAA) that recognizes structures containing α2,3-linked sialic acids, indicating that sialylated glycans were abundant on the hESC cell surface (FIG. 6A). Such glycans would thus be available for recognition by more specific glycan-recognizing reagents such as antibodies. In contrast, the cell surfaces were not labelled by Pisum sativum agglutinin (PSA) that recognizes α-mannosylated glycans (FIG. 6B). However, PSA labelled the cells after permeabilization (data not shown), suggesting that the majority of the mannosylated N-glycans in hESC were localized in intracellular cell compartments such as ER or Golgi (FIG. 6C). Interestingly, the mouse fibroblast cells showed complementary staining patterns compared to hESC, suggesting that these lectin reagents efficiently discriminated between hESC and feeder cells. Together the results suggested that the glycan structures we identified could be utilized to design reagents specifically targeting undifferentiated hESC.

Discussion

In the present study, novel glycan analysis methods were applied in the first structural analysis of hESC N-glycan profiles. By employing efficient purification of non-derivatized glycans we demonstrated mass spectrometric N-glycan profiles of the scarce hESC and differentiated cell samples from approximately 100 000 cells. As a result, dramatic glycan profile differences were discovered between the analyzed cell types. The objective in the present study was to provide a global view on the N-glycome profile, or a “fingerprint” of hESC N-glycosylation, rather than to present the stem cell glycome in terms of the molecular structures of each glycan component. The structural information already allowed us to determine the most abundant N-glycan structures of hESC. Furthermore, changes observed in the N-glycan profiles provided vast amount of information regarding hESC N-glycosylation and its changes during differentiation, allowing rational design of detailed structural studies of selected glycan components. It will be of great interest to apply these glycan analysis methods to other stem cell and differentiated cell types.

The results indicated that a defined group of N-glycan signals dominates the hESC N-glycome forming a unique stem cell glycan profile. For example, the fifteen most abundant neutral N-glycan signals and fifteen most abundant sialylated N-glycan signals in hESC together comprised over 85% of the N-glycome. On the other hand, structurally different glycan structures were favored during hESC differentiation. This suggests that N-glycan biosynthesis in hESC is a controlled and predetermined process.

Based on our results the hESC N-glycome seems to contain both a constant part consisting of “housekeeping glycans”, and a changeable part that is altered when the hESC differentiate (FIG. 2). The constant part seems to contain mostly high-mannose type and biantennary complex-type N-glycans, which may need to be present at all times for the maintenance of fundamental cellular processes. Significantly, 25% of the total N-glycan profile of hESC changed during their differentiation (see Supplementary FIG. S4). This indicates that during differentiation hESC dramatically change both their appearance towards their environment and possibly also their own capability to sense and respond to exogenous signals.

Our data show that the differentiation-associated change in the N-glycome was mostly generated by the addition or removal of variable epitopes on similar N-glycan core compositions. The present lectin staining experiments demonstrated that sialylated glycans were abundant on the cell surface of hESC, indicating that cell type specific N-glycan structures are potential targets for development of more specific recognition reagents. It seems plausible that knowledge of the changing surface glycan epitopes could be utilized as a basis in developing reagents and culture systems that would allow improved identification, selection, manipulation, and culture of hESC and their progeny. Protein-linked glycans perform their functions in cells by acting as ligands for specific glycan receptors (38-39), functioning as structural elements of the cell (40), and modulating the activity of their carrier proteins and lipids (2). More than half of all proteins in a human cell are glycosylated. Consequently, a global change in protein-linked glycan biosynthesis can simultaneously modulate the properties of multiple proteins. It is likely that the large changes in N-glycans during hESC differentiation have major influences on a number of cellular signaling cascades and affect in profound fashion biological processes within the cells.

The major hESC specific glycosylation feature we identified was the presence of more than one deoxyhexose residue in N-glycans, indicating complex fucosylation. Fucosylation is known to be important in cell adhesion and signalling events as well as being essential for embryonic development (41). Knock-out of the N-glycan core α1,6-fucosyltransferase gene FUT8 leads to postnatal lethality in mice (42), and mice completely deficient in fucosylated glycan biosynthesis do not survive past early embryonic development (43).

Fucosylated glycans such as the SSEA-1 antigen (7, 44-45) have previously been associated with both mouse embryonic stem cells (mESC) and human embryonic carcinoma cells (EC; 16), but not with hESC. The published gene expression profiles for the same hESC lines as studied here (46) have demonstrated that three human fucosyltransferase genes, FUT1, FUT4, and FUT8 are expressed in hESC, and that FUT1 and FUT4 are overexpressed in hESC when compared to EB. FUT8 encodes the N-glycan core α1,6-fucosyltransferase whose product was identified as the major fucosylated epitope in hESC N-glycans (FIG. 4B). The hESC-specific expression of FUT1 and FUT4, encoding for α1,2-fucosyltransferase and α1,3-fucosyltransferase enzymes (47), respectively, correlate with our findings of simple fucosylation in EB and complex fucosylation in hESC. Interestingly, the FUT4-encoded enzyme is capable of synthesizing the SSEA-1 antigen (48-49). Although hESC do not express the specific glycolipid antigen recognized by the SSEA-1 antibody, they share with mESC the characteristic feature of complex fucosylation and may also share the conserved essential biological functions of fucosylated glycan epitopes.

New N-glycan forms also emerged in EB and stage 3 differentiated cells. These structural features included additional N-acetylhexosamine residues, potentially leading to new N-glycan terminal epitopes. Another differentiation-associated feature was increase in the molar proportions of hybrid-type or monoantennary N-glycans. Biosynthesis of hybrid-type and complex-type N-glycans has been demonstrated to be biologically significant for embryonic and postnatal development in the mouse (50-51). The preferential expression of complex-type N-glycans in hESC and then the change in the differentiating EB to express more hybrid-type or monoantennary N-glycans may be significant for the process of stem cell differentiation.

Human embryonic stem cell lines have previously been demonstrated to have a common genetic stem cell signature that can be identified using gene expression profiling techniques (17,52-54). Such signatures have been proposed to be useful in hESC characterization. In the present report we provide the first glycomic signatures for hESC. The profile of the expressed N-glycans might be a useful tool for analyzing and classifying the differentiation stage in association with gene and protein expression analyses. Here we demonstrated that a glycan score algorithm was able to reliably differentiate the cell samples in separate differentiation stages (FIG. 5). Glycome profiling might be more sensitive than the use of any single cell surface marker and especially useful for the quality control of hESC-based cell products. However, further analysis of the hESC glycome may also lead to discovery of novel glycan antigens that could be used as stem cell markers in addition to the commonly used SSEA and Tra glycan antigens.

In conclusion, hESC have a unique N-glycome which undergoes major changes when the cells differentiate. Information regarding the specific glycan structures may be utilized in developing reagents for targeting these cells and their progeny. Future studies investigating the developmental and molecular regulatory processes resulting in the observed N-glycan profiles may provide significant insight into mechanisms of human development and regulation of glycosylation.

### REFERENCES FOR EXAMPLE 1

1. Shriver, Z., Raguram, S., and Sasisekharan, R. (2004) Nat. Rev. Drug Disc. 3, 863-873

2. Varki, A. (1993) Glycobiology 3, 97-130

3. Apweiler, R., Hermjakob, H., and Sharon, N. (1999) Biochim Biophys. Acta 1473, 4-8

4. Lowe, J. B. (2002) Immunol. Rev. 186, 19-36

5. Fukuda, M. (2002) Biochim. Biophys. Acta 1573, 394-405

6. Dell, A., Morris, H. R., Easton, R. L., Patankar, M., and Clark, G. F. (1999) Biochim. Biophys. Acta 1473, 196-205

7. Fenderson, B. A., Zehavi, U., and Hakomori, S. (1984) J. Exp. Med. 160, 1591-1596

8. Handel, T. M., Johnson, Z., Crown, S. E., Lau, E. K., and Proudfoot, A. E. (2005) Annu. Rev. Biochem. 74, 385-410

9. Helenius, A., and Aebi, M. (2001) Science 291, 2364-2369

10. Helenius, A., and Aebi, M. (2004) Annu. Rev. Biochem. 73, 1019-1049

11. Kornfeld, S. (1986) J. Clin. Invest. 77, 1-6

12. Thomson, J. A., Itskovitz-Eldor, J., Shapiro, S. S., Waknitz, M. A., Swiergiel, J. J., Marshall, V. S., and Jones, J. M. (1998) Science 282, 1145-1147

13. Wobus, A. M., and Boheler, K. R. (2005) Physiol. Rev. 85, 635-678

14. Kannagi, R., Cochran, N. A., Ishigami, F., Hakomori, S., Andrews, P. W., Knowles, B. B., and Solter, D. (1983) EMBO J. 2, 2355-2361

15. Badcock, G., Pigott, C., Goepel, J., and Andrews, P. W. (1999) Cancer Res. 59, 4715-4719

16. Muramatsu, T., and Muramatsu, H. (2004) Glycoconj. J. 21, 41-45

17. Mikkola, M., Olsson, C., Palgi, J., Ustinov, J., Palomäki, T., Horelli-Kuitunen, N., Knuutila, S., Lundin, K., Otonkoski, T., Tuuri, T. (2006) BMC Dev. Biol. 6, 40

18. Okabe, S., Forsberg-Nilsson, K., Spiro, A. C., Segal, M., and McKay, R. D. (1996) Mech. Dev. 59, 89-102

19. Nyman, T. A., Kalkkinen, N., Tölö, H., and Helin, J. (1998) Eur. J. Biochem. 253, 485-493

20. Verostek, M. F., Lubowski, C., and Trimble, R. B. (2000) Anal. Biochem. 278, 111-122

21. Davies, M. J., Smith, K. D., Carruthers, R. A., Chai, W., Lawson, A. M., and Hounsell, E. F. (1993) J. Chromatogr. 646, 317-326

22. Saarinen, J., Welgus, H. G., Flizar, C. A., Kalkkinen, N., and Helin, J. (1999) Eur. J. Biochem. 259, 829-840

23. Harvey, D. J. (1993) Rapid Commun. Mass Spectrom. 7, 614-619

24. Naven, T. J., and Harvey, D. J. (1996) Rapid Commun. Mass Spectrom. 10, 1361-1366

25. Papac, D. I., Wong, A., and Jones, A. J. (1996) Anal. Chem. 68, 3215-3223

26. Dell, A., and Morris, H. R. (2001) Science 291, 2351-2356

27. Sutton-Smith, M., Morris, H. R., Grewal, P. K., Hewitt, J. E., Bittner, R. E., Goldin, E., Schiffmann, R., and Dell, A. (2002) Biochem. Soc. Symp. 69, 105-115

28. Novotny, M. V., and Mechref, Y. J. (2005) Sep. Sci. 28, 1956-1968

29. Uematsu, R., Furukawa, J., Nakagawa, H., Shinohara, Y., Deguchi, K., Monde, K., and Nishimura, S. (2005) Mol. Cell. Proteomics 4, 1977-1989

30. Callewaert, N., Van Vlierberghe, H., Van Hecke, A., Laroy, W., Delanghe, J., and Contreras, R. (2004) Nat. Med. 10, 429-434

31. Martin, M. J., Muotri, A., Gage, F., and Varki, A. (2005) Nat. Med. 11, 228-232

32. Heiskanen, A., Satomaa, T., Tiitinen, S., Laitinen, A., Mannelin, S., Impola, U., Mikkola, M., Olsson, C., Miller-Podraza, H., Blomqvist, M., Olonen, A., Salo, H., Lehenkari, P., Tuuri, T., Otonkoski, T., Natunen, J., Saarinen, J., Laine, J. Stem Cells, in press.

33. Kornfeld, R., and Kornfeld, S. (1985) Annu. Rev. Biochem. 54, 631-664

34. Schachter, H. (1991) Glycobiology 1, 453-461

35. Staudacher, E., Altmann, F., Wilson, I. B. H., and März, L. (1999) Biochim. Biophys. Acta 1473, 216-346

36. Venable, A., Mitalipova, M., Lyons, I., Jones, K., Shin, S., Pierce, M., and Stice, S. (2005) BMC Dev. Biol. 5, 15.

37. Wearne, K. A., Winter, H. C., O'Shea, K., and Goldstein, I. J. (2006) Glycobiology, in press

38. Kilpatrick, D. C. (2002) Biochim. Biophys. Acta 1572, 187-197

39. Zanetta, J. P., and Vergoten, G. (2003) Adv. Exp. Med. Biol. 535, 107-124

40. Imperiali, B., and O'Connor, S. E. (1999) Curr. Opin. Chem. Biol. 3, 643-649

41. Becker, D. J., and Lowe, J. B. (2003) Glycobiology 13:41R-53R

42. Wang, X., Inoue, S., Gu, J., Miyoshi, E., Noda, K., Li, W., Mizuno-Horikawa, Y., Nakano, M., Asahi, M., Takahashi, M., Uozumi, N., Ihara, S., Lee, S. H., Ikeda, Y., Yamaguchi, Y., Aze, Y., Tomiyama, Y., Fujii, J., Suzuki, K., Kondo, A., Shapiro, S. D., Lopez-Otin, C., Kuwaki, T., Okabe, M., Honke, K., and Taniguchi, N. (2005) Proc. Natl. Acad. Sci. U.S.A. 102:15791-15796

43. Smith, P. L., Myers, J. T., Rogers, C. E., Zhou, L., Petryniak, B., Becker, D. J., Homeister, J. W., and Lowe, J. B. (2002) J. Cell Biol. 158, 801-815

44. Solter, D., and Knowles, B. B. (1978) Proc. Natl. Acad. Sci. U.S.A. 75, 5565-5569

45. Gooi, H. C., Feizi, T., Kapadia, A., Knowles, B. B., Solter, D., and Evans, M. J. (1981) Nature 292, 156-158

46. Skottman, H., Mikkola, M., Lundin, K., Olsson, C., Strömberg, A. M., Tuuri, T., Otonkoski, T., Hovatta, O., and Lahesmaa, R. (2005) Stem cells 23, 1343-1356

47. Mollicone, R., Cailleau, A., and Oriol, R. (1995) Transfusion Clin. Biol. 4:235-242

48. Nakayama, F., Nishihara, S., Iwasaki, H., Kudo, T., Okubo, R., Kaneko, M., Nakamura, M., Karube, M., Sasaki, K., and Narimatsu, H. (2001) J. Biol. Chem. 276, 16100-16106

49. Kudo, T., Kaneko, M., Iwasaki, H., Togayachi, A., Nishihara, S., Abe, K., and Narimatsu, H. (2004) Mol. Cell. Biol. 24, 4221-4228

50. Ioffe, E., and Stanley, P. (1994) Proc. Natl. Acad. Sci. U.S.A. 91, 728-732

51. Metzler, M., Gertz, A., Sarkar, M., Schachter, H., Schrader, J. W., and Marth, J. D. (1994) EMBO J. 13, 2056-2065

52. Wang, Y., Tan, J., Sutton-Smith, M., Ditto, D., Panico, M., Campbell, R. M., Varki, N. M., Long, J. M., Jaeken, J., Levinson, S. R., Wynshaw-Boris, A., Morris, H. R., Le, D., Dell, A., Schachter, H., and Marth, J. D. (2001) Glycobiology 11, 1051-1070

53. Akama, T. O., Nakagawa, H., Wong, N. K., Sutton-Smith, M., Dell, A., Morris, H. R., Nakayama, J., Nishimura, S., Pai, A., Moremen, K. W., Marth, J. D., and Fukuda, M. N. (2006) Proc. Natl. Acad. Sci. U.S.A. 103, 8983-8988

54. Sato, N., Sanjuan, I. M., Heke, M., Uchida, M., Naef, F., and Brivanlou, A. H. (2003) Dev. Biol. 260, 404-413

55. Abeyta, M. J., Clark, A. T., Rodriguez, R. T., Bodnar, M. S., Pera, R. A., and Firpo, M. T. (2004) Hum. Mol. Genet. 13, 601-608

56. Bhattacharya, B., Miura, T., Brandenberger, R., Mejido, J., Luo, Y., Yang, A. X., Joshi, B. H., Ginis, I., Thies, R. S., Amit, M., Lyons, I., Condie, B. G., Itskovitz-Eldor, J., Rao, M. S., and Puri, R. K. (2004) Blood 103, 2956-2964

### Example 2

### Analysis of N-Glycan Composition Groups with Terminal HexNAc in Stem Cells and Differentiated Cells

Methods. To analyze the presence of terminal HexNAc containing N-glycans characterized by the formulae: n_{HexNAc}=n_{Hex}≧5 and n_{dHex}≧1 (group I), and to compare their occurrence to terminal HexNAc containing N-glycans characterized by the formulae: n_{HexNAc}=n_{Hex}≧5 and n_{dHex}=0 (group II), N-glycans were isolated, purified and analyzed by MALDI-TOF mass spectrometry as described in the preceding Examples. They were assigned monosaccharide compositions and their relative proportions within the obtained glycan profiles were determined by quantitative profile analysis as described above. The following glycan signals were used as indicators of the specific glycan groups (monoisotopic masses):

Ia, Hex₅HexNAc₅dHex₁: m/z for [M+Na]+ ion 2012.7

Ib, NeuAc₁Hex₅HexNAc₅dHex₁: m/z for [M−H]− ion 2279.8

Ic, NeuAc₂Hex₅HexNAc₅dHex₁: m/z for [M−H]− ion 2570.9

Id, NeuAc₁Hex₅HexNAc₅dHex₂: m/z for [M−H]− ion 2425.9

IIa, NeuAc₁Hex₅HexNAc₅: m/z for [M−H]− ion 2133.8

Further, relative expression of glycan signals Hex₃HexNAc₅: m/z for [M+Na]+ ion 1542.6 and Hex₃HexNAc₅dHex₁: m/z for [M+Na]+ ion 1688.6 was also analyzed.

Results. As an indicator of group I glycans, Ib was detected in various N-glycan samples isolated from stem cell samples, including EB and st.3 differentiated cells.

hESC lines FES 22, FES 29, and FES 30: Ia, Ib, Ic, Id, and IIa were overexpressed in EB and st.3 when compared to hESC. Specifically, Ia was not expressed in hESC and IIa was expressed in only ⅓ of the hESC samples. The relative abundance of Hex₃HexNAc₅ and Hex₃HexNAc₅dHex₁ was also increased in EB and st.3: for Hex₃HexNAc₅ by 6.1 fold and 7.8 fold, and for Hex₃HexNAc₅dHex₁ by 1.2 fold and 2.6 fold for the transitions from hESC to EB and hESC to st.3, respectively.

### Example 3

### Evaluation of Individual Variation in Relative Proportions of N-Glycan Signals of hESC Lines

The propensity of each glycan signal to be subject to individual variation between cell lines was estimated by calculating the average deviation of the glycan signal relative proportions between the four hESC lines. The deviations were then evaluated as proportion of average deviation from the average signal proportion (in %). In this calculation, three groups of glycan signals were obtained: over 100% average deviation (large individual variation), between 50-100% average deviation (substantial individual variation), and between 0-50% average deviation (little individual variation). Below are the glycan signals listed in Tables 1 and 2 as grouped according to this.

Over 100% (large individual variation):

Neutral N-glycans H4N3F2, H5N5, H4N5, H4N5F2, H4N4F2, H6N4, H4N5F1, H5N5F1, H3N5, H2N4F1, H4N4, H4N5F3, H2N2, H3N5F1, H5N2F1, and H6N3F1.

Sialylated N-glycans S2H7N6F1, S2H4N3F1, S2H5N5F1, S1H5N5, S3H6N5, S2H6N5F2, S2H5N3F1, S2H3N3F1, S1H8N7F1, S1H6N4F2, S1H5N3F1, S2H6N4, S1H4N4F1, G2H5N4, and S1H6N4F1Ac.

Over 50% (moderate individual variation):

Neutral N-glycans H1N2, H11N2, H5N3F1, H5N4F3, H5N4F2, H3N2F1, N2N2F1, H6N3, and H3N2.

Sialylated N-glycans S2H5N4, S1H6N5F3, S2H4N5F1, S1H6N4F1, S1G1H5N4, S1H6N3, S1H5N3, S1H4N3, S1H7N6F2, G1H5N4, S2H2N3F1, S1H6N5, and S1H7N6F3.

Over 0% (little individual variation):

Neutral N-glycans H5N3, H5N4F1, H6N5F1, H3N3, H3N4F1, H4N2F1, H6N5, H3N3F1, H4N3, H4N2, H4N4F1, H5N4, H8N2, H4N3F1, H10N2, H5N2, H7N2, and H9N2.

Sialylated N-glycans S1H4N5F2, S1H7N6F1, S1H5N4F3, S1H5N5F2, S1H6N5F2, S1H4N5F1, S2H6N5F1, G1H5N4F1, S1H5N4F2, S2H5N4F1, S1H5N5F1, S1H6N5F1, S1H5N4, S1H4N3F1, and S1H5N4F1.

The major glycan signals were in the group of little individual variation. This group also included the major biantennary-size complex-type N-glycans including S1H5N4F1, the major high-mannose type N-glycans including H9N2, and the major complex-fucosylated complex-type N-glycans including S1H5N4F2 and S1H5N4F3, showing that these major hESC-associated glycan features were not subject to significant individual variation between hESC lines.

Cell line specific N-glycan profile data is presented in Tables 10 and 11, formatted as in Example 1.

### Example 4

### Analysis of N-Glycan, Glycolipid and O-Glycan Cellular Glycan Types by Specific Glycosidases and Mass Spectrometry

Assignment of Lewis x on N-glycans

Previously it was indicated by combination of NMR spectroscopy and β1,4-galactosidase, β-N-acetylglucosaminidase, and β-hexosaminidase digestions that hESC neutral monoantennary and biantennary-size N-glycans preferentially contained type 2 LacNAc antennae and also minor amounts of LacdiNAc antennae, more preferentially in a complex-type biantennary N-glycan backbone with β1,2-branches. Here it was studied by α1,3/4-fucosidase digestion of the hESC neutral N-glycan fraction which specific antennae contained α1,3-fucosylation decorations of these antennae. The glycan sample was produced as described in the other Examples of the present invention from similar hESC samples.

Monoantennary N-glycans that were digested with α1,3/4-fucosidase included H4N3F2 (m/z 1590), digested into H4N3F1 (1444), preferentially including the non-reducing terminal structure Lexβ2Man, more preferentially also including a complete N-glycan structure Lexβ2Manα3(Manα6)Manβ4GlcNAcβ4(Fucα6)GlcNAc.

Biantennary-size N-glycans that were digested with α1,3/4-fucosidase included H5N4F2 (m/z 1955) and H5N4F3 (2101), which were digested into H5N4F1 (1809); and H4N5F2 (1996) and H4N5F3 (2142), which were digested into H4N5F1 (1850). These glycans preferentially included the non-reducing terminal structures Lexβ2Man and GalNAcβ4(Fucα3)GlcNAcβ2Man, respectively, more preferentially also including complete N-glycan structures:

Lexβ2Manα3(Lexβ2Manα6)Manβ4GlcNAcβ4(Fucα6)GlcNAc and GalNAcβ4(Fucα3)GlcNAcβ2ManαX(Lexβ2ManαY)Manβ4GlcNAcβ4(Fucα6)GlcNAc, wherein X and Y are either 3 or 6, and X≠Y.

O-glycan and Glycolipid Analysis

The glycosphingolipid glycan and reducing O-glycan samples were isolated from studied cell types, analyzed by mass spectrometry, and further analyzed by expoglycosidase digestions combined with mass spectrometry as described in the present invention and the preceding Examples. Non-reducing terminal epitopes were analyzed by digestion of the glycan samples with S. pneumoniae β1,4-galactosidase (Calbiochem), bovine testes β-galactosidase (Sigma), A. ureafaciens sialidase (Calbiochem), S. pneumoniae α2,3-sialidase (Calbiochem), S. pneumoniae β-N-acetylglucosaminidase (Calbiochem), X. manihotis α1,3/4-fucosidase (Calbiochem), and α1,2-fucosidase (Calbiochem). The results were analyzed by quantitative mass spectrometric profiling data analysis as described in the present invention. The results with glycosphingolipid glycans are summarized in Table 22 including also core structure classification determined based on proposed monosaccharide compositions as described in the footnotes of the Table. Analysis of neutral O-glycan fractions revealed quantitative differences in terminal epitope glycosylation as follows: non-reducing terminal type 1 LacNAc (β1,3-linked Gal) had above 5% proportion is characteristic to hESC. Fucosylation degree of type 2 LacNAc containing O-glycan signals at m/z 771 (Hex₂HexNAc₂) and 917 (Hex₂HexNAc₂dHex₁) was 28% in hESC.

In conclusion, these results from O-glycans and glycosphingolipid glycans demonstrated significant cell type specific differences and also were significantly different from N-glycan terminal epitopes within each cell type analyzed in the present invention.

### Example 5

### Glycosphingolipid Glycans of Human Stem Cells

Experimental Procedures

Samples from hESC grown on mouse fibroblast feeder cells were produced as described in the preceding Examples. Neutral and acidic glycosphingolipid fractions were isolated from cells essentially as described (Miller-Podraza et al., 2000). Glycans were detached by Macrobdella decora endoglycoceramidase digestion (Calbiochem, USA) essentially according to manuacturer's instructions, yielding the total glycan oligosaccharide fractions from the samples. The oligosaccharides were purified and analyzed by MALDI-TOF mass spectrometry as described in the preceding Examples for the protein-linked oligosaccharide fractions.

Results and Discussion

Human Embryonic Stem Cells (hESC)

hESC neutral lipid glycans. The analyzed mass spectrometric profile of the hESC glycosphingolipid neutral glycan fraction was analyzed (not shown).

Structural analysis of the major neutral lipid glycans. The six major glycan signals, together comprising more than 90% of the total glycan signal intensity, corresponded to monosaccharide compositions Hex₃HexNAc₁ (730), Hex₃HexNAc₁dHex₁ (876), Hex₂HexNAc₁ (568), Hex₃HexNAc₂ (933), Hex₄HexNAc₁ (892), and Hex₄HexNAc₂ (1095).

In β1,4-galactosidase digestion, the relative signal intensities of 1095 and 730 were reduced by about 30% and 10%, respectively. This suggests that 730 and 1095 contain minor components with non-reducing terminal β1,4-Gal epitopes, preferably including the structures Galβ4GlcNAcLac and Galβ4GlcNAc[Hex₁HexNAc₁]Lac. The other major components were thus shown to contain other terminal epitopes. Further, the glycan signal Hex₅HexNAc₃ (1460) was digested to Hex₃HexNAc₃ (1136), indicating that the original signal contained glycan structures containing two β1,4-Gal.

The major glycan signals were not sensitive to α-galactosidase digestion.

In α1,3/4-fucosidase digestion, the signal intensity of 876 was reduced by about 10%, indicating that only a minor proportion of the glycan signal corresponded to glycans with α1,3- or α1,4-linked fucose residue. The major affected signal in the total profile was Hex₃HexNAc₁dHex₂ (1022), indicating that it included glycans with either α1,3-Fuc or α1,4-Fuc. 511 was reduced by about 30%, indicating that the signal contained a minor component with α1,2-Fuc, preferentially including Fucα2Galβ4Glc (Fucα2′Lac, 2′-fucosyllactose).

When the α1,3/4-fucosidase reaction product was further digested with α1,2-fucosidase, 876 was completely digested into 730, indicating that the structure of the majority of the signal intensity contained non-reducing terminal α1,2-Fuc, preferably including the structure Fucα2[Hex₁HexNAc₁]Lac, more preferably including Fucα2GalHexNAcLac. Another partly digested glycan signal was Hex₄HexNAc₂dHex₁ (1241) that was thus indicated to contain α1,2-Fuc, preferably including the structure Fucα2[Hex₂HexNAc₂]Lac, more preferably including Fucα2Gal[Hex₁HexNAc₂]Lac. 511 was completely digested, indicating that the original signal contained a major component with α1,3/4-Fuc, preferentially including Galβ4(Fucα3)Glc (3-fucosyllactose).

When the α1,3/4-fucosidase and α1,2-fucosidase reaction product was further digested with β1,4-galactosidase, the majority of the newly formed 730 was not digested, i.e. the relative proportion of 568 was not increased compared to β1,4-galactosidase digestion without preceding fucosidase treatments. This indicated that the majority of 876 did not contain β1,4-Gal subterminal to Fuc. Further, 892 was not digested, indicating that it did not contain non-reducing terminal β1,4-Gal.

When the α1,3/4-fucosidase, α1,2-fucosidase, and β1,4-galactosidase reaction product was further digested with β1,3-galactosidase, the signal intensity of 892 was reduced, indicating that it included glycans with terminal β1,3-Gal. The signal intensity of 568 was increased relative to 730, indicating that also 730 included glycans with terminal β1,3-Gal.

The experimental structures of the major hESC glycosphingolipid neutral glycan signals were thus determined (‘>’ indicates the order of preference among the lipid glycan structures of hESC; ‘[ ]’ indicates that the oligosaccharide sequence in brackets may be either branched or unbranched; ‘( )’ indicates a branch in the structure):
730 Hex₃HexNAc₁>Hex₁HexNAc₁Lac>Galβ4GlcNAcLac
876 Hex₃HexNAc₁dHex₁>Fucα2[Hex₁HecNAc₁]Lac>Fucα2Galβ4GlcNAcLac>Fucα3/4[Hex₁HecNAc₁]Lac
568 Hex₂HexNAc₁>HecNAcLac
933 Hex₃HexNAc₂>[Hex₁HecNAc₂]Lac
892 Hex₄HexNAc₁>[Hex₂HecNAc₁]Lac>Galβ3[Hex₁HecNAc₁]Lac
1095 Hex₄HexNAc₂>[Hex₂HecNAc₂]Lac>Galβ3HexNAc[Hex₁HecNAc₁]Lac>Galβ4GlcNAc[Hex₁HecNAc₁]Lac
1460 Hex₅HexNAc₃>[Hex₃HecNAc₃]Lac>Galβ4GlcNAc(Galβ4GlcNAc)[Hex₁HecNAc₁]Lac

Acidic lipid glycans. The mass spectrometric profile of the hESC glycosphingolipid sialylated glycan fraction was analyzed (not shown). The four major glycan signals, together comprising more than 96% of the total glycan signal intensity, corresponded to monosaccharide compositions NeuAc₁Hex₃HexNAc₁ (997), NeuAc₁Hex₂HexNAc₁ (835), NeuAc₁Hex₄HexNAc₁ (1159), and NeuAc₂Hex₃HexNAc₁ (1288).

The acidic glycan fraction was subjected to α2,3-sialidase digestion and the resulting neutral and acidic glycan fractions were purified and analyzed separately. In the acidic fraction, signals 1159 and 1288 were digested and 835 was partly digested. In the neutral fraction, signals 730 and 892 were the major appeared signals. These results indicated that: 1159 consisted mainly of glycans with α2,3-NeuAc, 1288 contained at least one α2,3-NeuAc, a major proportion of glycans in 835 contained α2,3-NeuAc, and in the original sample a major proportion of NeuAc₁₋₂Hex₃HexNAc₁ contained solely α2,3-linked NeuAc.

### Example 6

### Endo-β-galactosidase Analysis of Cellular Glycan Types

Endo-β-galactosidase Reaction Conditions

The substrate glycans were dried in 0.5 ml reaction tubes. The endo-β-galactosidase (E. freundii, Seikagaku Corporation, cat no 100455, 2.5 mU/reaction) reactions were carried out in 50 mM Na-acetate buffer, pH5.5 at 37° C. for 20 hours. After the incubation the reactions mixtures were boiled for 3 minutes to stop the reactions. The substrate glycans were purified using chromatographic methods according to the present invention, and analyzed with MALDI-TOF mass spectrometry as described in the preceding Examples.

In similar reaction conditions with with 2 nmol of each defined oligosaccharide control, the reaction produced signal at m/z 568 (Hex₂HexNAc₁) as the major reaction product from lacto-N-neotetraose and para-lacto-N-neohexaose, but not from lacto-N-neohexaose or para-lacto-N-neohexaose monofucosylated at the 3-position of the inner GlcNAc residue; and sialylated signal corresponding to NeuAc₁Hex₂HexNAc₁ from α3′-sialyl-lacto-N-neotetraose. These results confirmed the reported specificities for the enzyme in the employed reaction conditions.

Results with Cellular Glycan Types

hESC O-glycans. In neutral reducing O-glycans isolated from hESC, major digestion products were signals at m/z 568 (Hex₂HexNAc₁) and 714 (Hex₂HexNAc₁dHex₁), corresponding to non-fucosylated and fucosylated non-reducing glycan fragments from poly-N-acetyllactosamine sequences (poly-LacNAc); and at m/z 609 (Hex₁HexNAc₂) corresponding to another type of glycan fragment, including reducing end O-glycan fragment such as Core 2 trisaccharide Galβ3(GlcNAcβ6)GalNAc.

Major digested glycan signals corresponding to O-glycan structures were at m/z 1136 (Hex₃HexNAc₃), 974 (Hex₂HexNAc₃), 1120 (Hex₂HexNAc₃dHex₁), and 1282 (Hex₃HexNAc₃dHex₁). Signal 1136 corresponded to a glycan also sensitive to β1,3-galactosidase exoglycosidase digestion, and therefore was determined to contain a non-reducing end Galβ3GlcNAcβ3Galβ4GlcNAcβ sequence; signal 1282 corresponds to a fucosylated derivative thereof. Signals 974 and 1120 are non-fucosylated and fucosylated forms of O-glycans with non-reducing terminal HexNAc.

hESC glycosphingolipid glycans. The major digestion product in hESC neutral glycosphingolipid glycans were the signals at m/z 568 (Hex₂HexNAc₁) and 714 (Hex₂HexNAc₁dHex₁) indicating the presence of non-fucosylated and fucosylated poly-LacNAc sequences. Further, the signals at m/z 1428 (Hex₃HexNAc₃dHex₂) and 1282 (Hex₃HexNAc₃dHex₁) were products, indicating the presence of different glycan terminal sequences with non-reducing terminal HexNAc than in the abovementioned cell types. Major sensitive signals were signals at m/z 730, 876, 933, 1095, and 1241 with similar interpretation as with CB MNC above.

In conclusion, the profiles of endo-β-galactosidase reaction products efficiently reflected cell type specific glycosylation features as described in the preceding Examples and they represent an alternative and complementary method for analysis of cellular glycan types. Further, the present results demonstrated the presence of linear, branched, and fucosylated poly-LacNAc in all studied cell types and in different glycan types including N- and O-glycans and glycosphingolipid glycans; and further quantitative and cell-type specific proportions of these in each cell type, which are characteristic to each cell type.

hESC N-glycans. Combination of NMR spectroscopy and β1,4-galactosidase, β-N-acetylglucosaminidase, and β-hexosaminidase digestions indicates that hESC neutral monoantennary and biantennary-size N-glycans preferentially contained LacNAc (LN) antennae, more preferentially in a complex-type biantennary N-glycan backbone with β1,2-branches. Here it was studied by endo-β-galactosidase digestion of the hESC acidic N-glycan fraction, which N-glycan backbones contained poly-N-acetyllactosamine (poly-LN) antennae. The glycan sample was produced as described in the other Examples of the present invention from similar hESC samples.

Biantennary N-glycan fragments that were produced with endo-β-galactosidase included S1H4N4 (m/z 1917), preferentially produced from a biantennary N-glycan with one poly-LN antenna and one sialylated LN antenna. According to the present invention this glycan included an antenna structure R-GlcNAcβ3Galβ4GlcNAcβ2Man, wherein R is non-reducing N-glycan antenna structure according to the invention. In a further embodiment of the present invention, the other antenna in the same N-glycan is sialylated LacNAc, more preferably NeuAc-Gal-GlcNAcβ2Man.

### Example 7

### The Glycome of Human Embryonic Stem Cells Reflects their Differentiation Stage

Summary

Complex carbohydrate structures, glycans, are elementary components of glycoproteins, glycolipids, and proteoglycans. These glycoconjugates form a layer of glycans that covers all human cell surfaces and forms the first line of contact towards the cell's environment. Glycan structures called stage specific embryonic antigens (SSEA) are used to assess the undifferentiated stage of embryonic stem cells. However, the whole spectrum of stem cell glycan structures has remained unknown, largely due to lack of suitable analysis technology. We describe the first global study of glycoprotein glycans of human embryonic stem cells, embryoid bodies, and further differentiated cells by MALDI-TOF mass spectrometric profiling. The analysis reveals how certain asparagine-linked glycan structures characteristic to stem cells are lost during differentiation while new structures emerge in the differentiated cells. The results indicate that human embryonic stem cells have a unique glycome and that their differentiation stage can be identified by glycome analysis. We suggest that knowledge about stem cell specific glycan structures can be used for e.g. purification, manipulation, and quality control of stem cells.

### Materials & Methods

Human embryonic stem cell lines. Five Finnish hESC lines, FES 21, FES 22, FES 29, FES 30 (Skottman et al., 2005. Stem cells 23:1343-56) and FES 61 were used in the present study. These lines are included in the International Stem Cell Initiative (Andrews et al., 2005. Nat. Biotechnol. 23:795-7). The cells were propagated on human foreskin fibroblast (hFF) feeder cells in serum-free medium (Knockout™, Gibco/Invitrogen). In FACS analyses 70-90% of cells from mechanically isolated colonies were typically Tra 1-60 and Tra 1-81 positive (not shown). Cells differentiated into embryoid bodies (EB, stage 2 differentiated) and further differentiated cells grown out of the EB as monolayers (stage 3 differentiated) were used for comparison against hESC. The differentiation protocol favors the development of neuroepithelial cells while not directing the differentiation into distinct terminally differentiated cell types (Okabe et al., 1996. Mech. Dev. 59:89-102). EB derived from FES 30 had less differentiated cell types than the other three EB. Stage 3 cultures consisted of a heterogenous population of cells dominated by fibroblastoid and neuronal morphologies. For the glycome studies the cells were collected mechanically, washed, and stored frozen until analysis.

In a preferred embodiment the invention is directed to the use of data obtained embryoid bodies or ESC-cell line cultivated under conditions favouring neuroepithelial cells for search of specific structures indicating neuroepithelial development, preferably by comparing the material with cell materials comprising neuronal and/or epithelial type cells.

Asparagine-linked glycome profiling. Total asparagine-linked glycan (N-glycan) pool was enzymatically isolated from about 100 000 cells. The total N-glycan pool (picomole quantities) was purified with microscale solid-phase extraction and divided into neutral and sialylated N-glycan fractions. The N-glycan fractions were analyzed by MALDI-TOF mass spectrometry either in positive ion mode for neutral N-glycans or in negative ion mode for sialylated glycans (Saarinen et al., 1999, Eur. J. Biochem. 259, 829-840). Over one hundred N-glycan signals were detected from each cell type revealing the surprising complexity of hESC glycosylation. The relative abundances of the observed glycan signals were determined based on relative signal intensities (Harvey, 1993. Rapid Commun. Mass Spectrom. 7:614-9; Papac et al., 1996. Anal. Chem. 68:3215-23).

Results

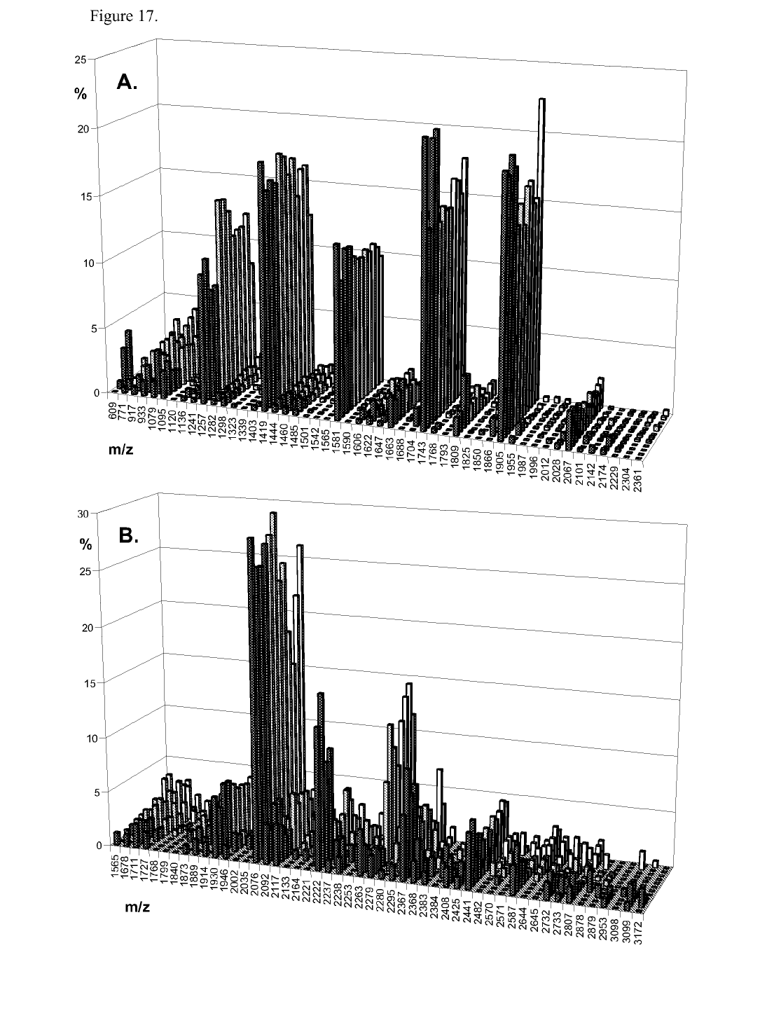

In the present study, we analyzed the N-glycome profiles of hESC, EB, and st.3 differentiated cells (FIG. 17).

The similarity of the N-glycan profiles within the group of four hESC lines suggested that the obtained N-glycan profiles are a description of the characteristic N-glycome of hESC. Overall, 10% of the 100 most abundant N-glycan signals present in hESC disappeared in st.3 differentiated cells, and 16% of the most abundant signals in st.3 differentiated cells were not present in hESC. This indicates that differentiation induced the appearance of new N-glycan types while earlier glycan types disappeared. In quantitative terms, the differences between the glycan profiles of hESC, EB, and st.3 differentiated cells were: hESC vs. EB 19%, hESC vs. st.3 24%, and EB vs. st.3 12%.

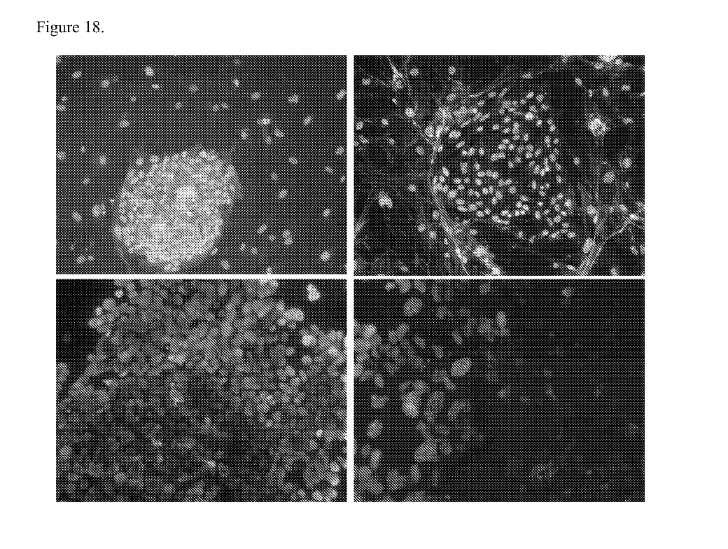

The glycome profile data was used to design glycan-specific labeling reagents for hESC. The most interesting glycan types were chosen to study their expression profiles by lectin histochemistry as exemplified in FIG. 18 for the lectins that recognize either α2,3-sialylated (MAA-lectin, FIG. 18A.) binding to the hESC cells or α-mannosylated glycans (PSA-lectin, FIG. 18B.) binding to the surfaces of feeder cells (MEF). The binding of the lectin reagents was inhibited by specific carbohydrate inhibitors, sialylα2-lactose and mannose, respectively (FIGS. 18C. and 18D.). The results are summarized in Table 43.

Table 43 further represent differential recognition feeder and stem cells by two other lectins, Ricinus communis agglutinin (RCA, ricin lectin), known to recognize especially terminal Galβ-structures, especially Galβ4Glc(NAc)-type structures and peanut agglutinin (PNA) recognizing Gal/GalNAc structures. The cell surface expression of ligand for two other lectin RCA and PNA on hESC cells, but only RCA ligands of feeder cells.

The present results indicate and the invention is directed to the hESC glycans are potential targets for recognition by stem cell specific reagents. The invention is further directed to methods of specific recognition and/or separation of hESC and differentiated cells such as feeder cells by glycan structure specific reagents such as lectins. Human embryonic stem cells have a unique glycome that reflects their differentiation stage. The invention is specifically directed to analysis of cells according to the invention with regard to differentiation stage.

The results were also used to generate an algorithm for identification of hESC differentiation stage (FIG. 5). To test whether the obtained N-glycan profiles could be used for reliable identification of hESC and differentiated cells even with the presence of sample-to-sample variation, a discrimination analysis was performed on the data. The hESC line FES 29 and embryoid bodies derived from it (EB 29) were selected as the training group for the calculation that effectively discriminated the two samples (FIG. 5):

glycan score=a−b−c,

wherein a is the sum of the relative abundances (%) of all signals with proposed compositions with two or more dHex (F≧2) in the sialylated N-glycan fraction, b is the sum of the relative abundances (%) of all signals with hybrid-type structures (ST=H), and c is the sum of the relative abundances (%) of all signals with proposed compositions with five or more HexNAc and equal amounts of Hex and HexNAc (H═N≧5); see Table 43 for structure codes and FIG. 17 for the dataset.

The resulting equation was applied to the other samples that served as the test group in the analysis and the results are described graphically in FIG. 5. hESC and the differentiated cell samples were clearly discriminated from each other (p<0.01, Student's t test). Furthermore, the st.3 differentiated cell samples were separated from the EB samples (p<0.05, Mann-Whitney test). The predicted 95% confidence intervals (assuming normal distribution of glycan scores within each cell type) are shown for the three cell types, indicating that a calculated glycan score has potential to discriminate all three cell types. At 96% confidence interval, hESC and the differentiated cell types (EB and st.3) were still discriminated from each other (not shown in the figure). The results indicate that glycome profiling is a tool for monitoring the differentiation status of stem cells.

Conclusions

The present data represent the glycome profiling of hESC:
hESC have a unique N-glycome comprising of over 100 glycan components
Differentiation induces a major change in the N-glycome and the cell surface molecular landscape of hESC

Utility of hESC glycome data:
Identification of new stem cell markers for e.g. antibody development
Quality control of stem cell products
Identification of hESC differentiation stage
Control of variation between hESC lines
Effect of external factors and culture conditions on hESC status

Especially preferred uses of the data are

Use of the hESC glycome for identification of specific cell surface markers characteristic for the pluripotent hESCs.

The invention is directed to further analysis and production of present and analogous glycome data and use of the methods for further indentification of novel stem cell specific glycosylation features and form the basis for studies of hESC glycobiology and its eventual applications according to the invention

### Example 8

### Identification of Specific Glycosylation Signatures from Glycan Profiles in Various Steps of Human Embryonic Stem Cell Differentiation

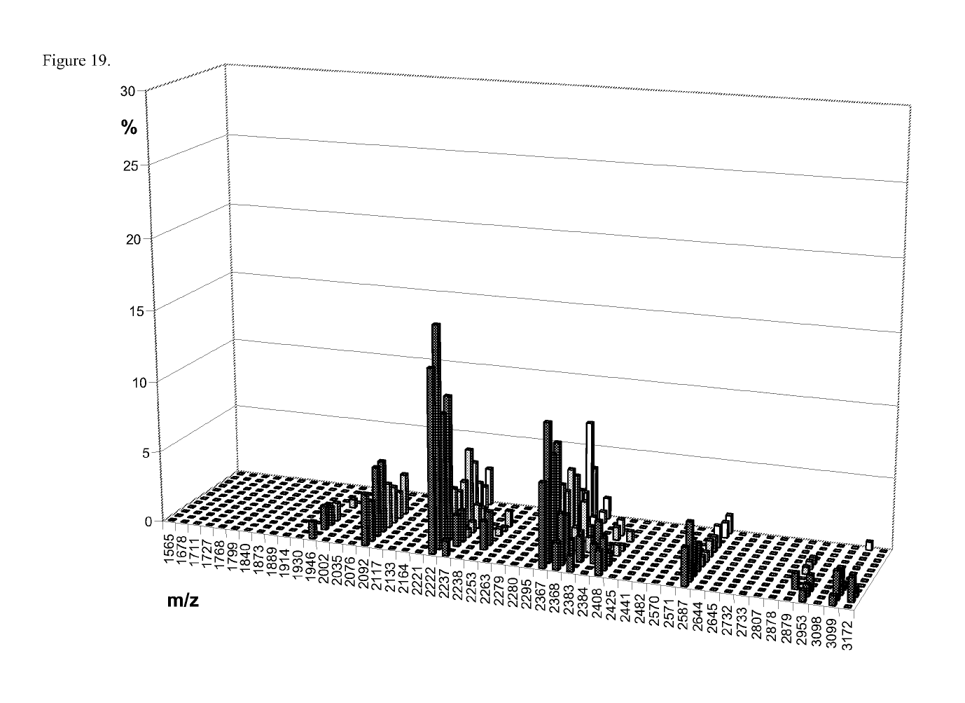

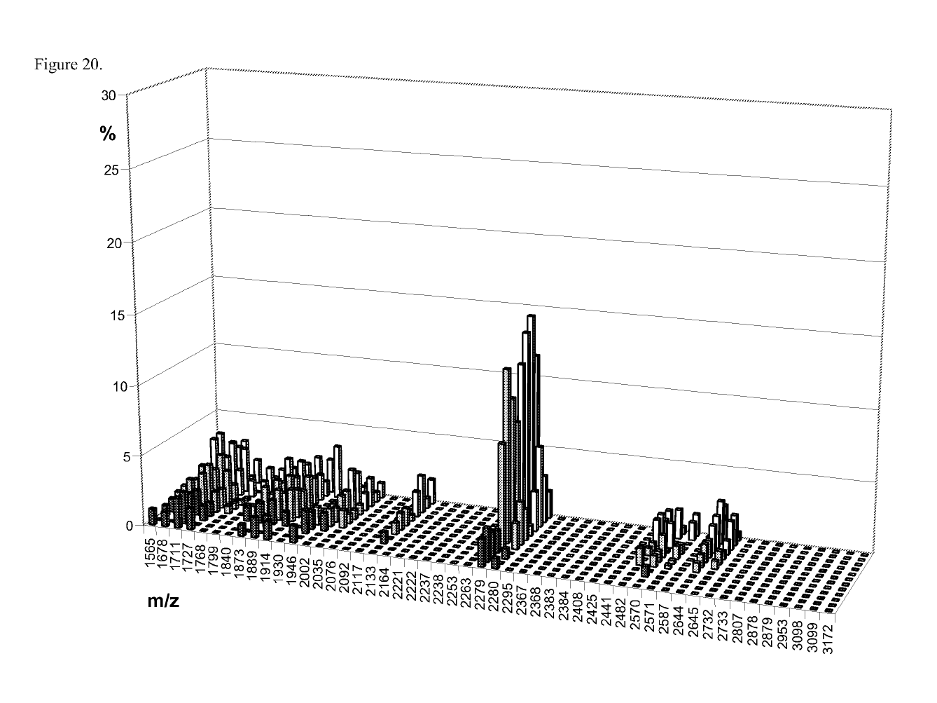

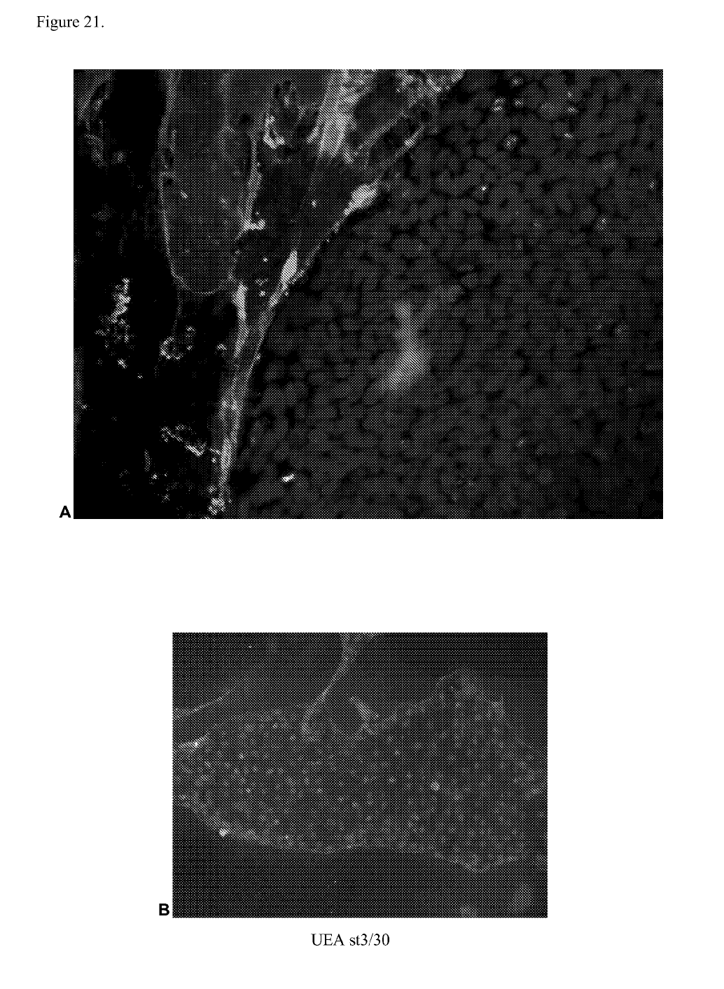

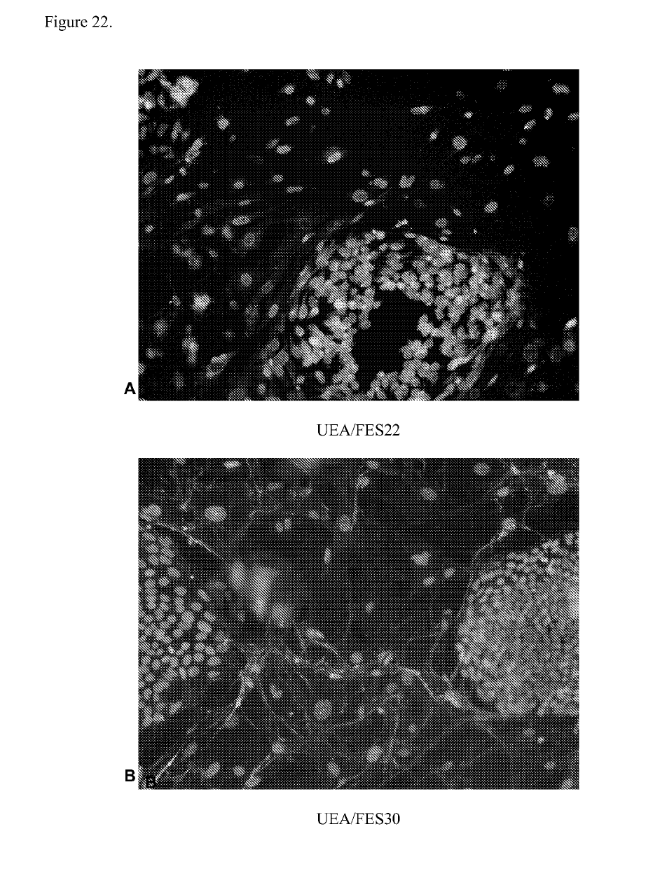

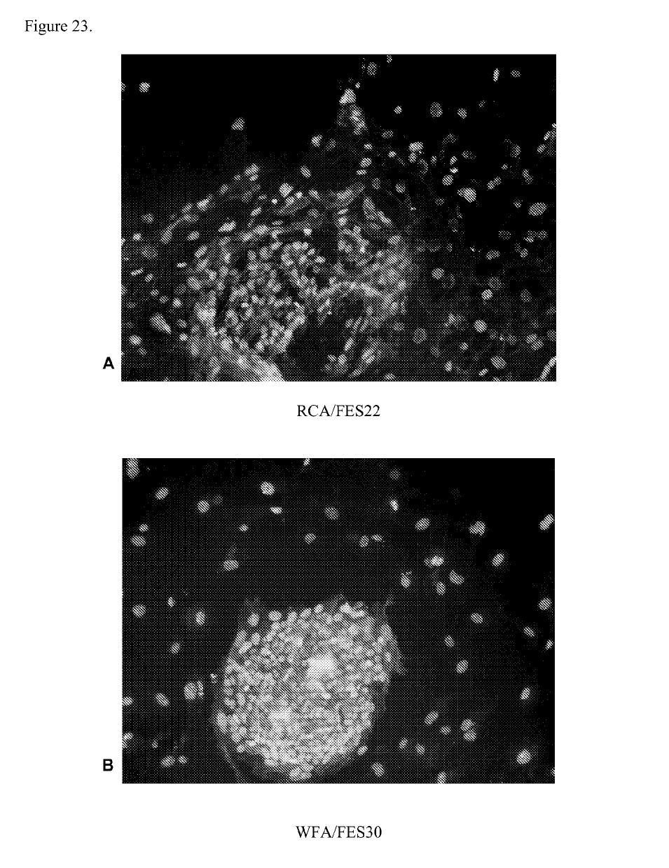

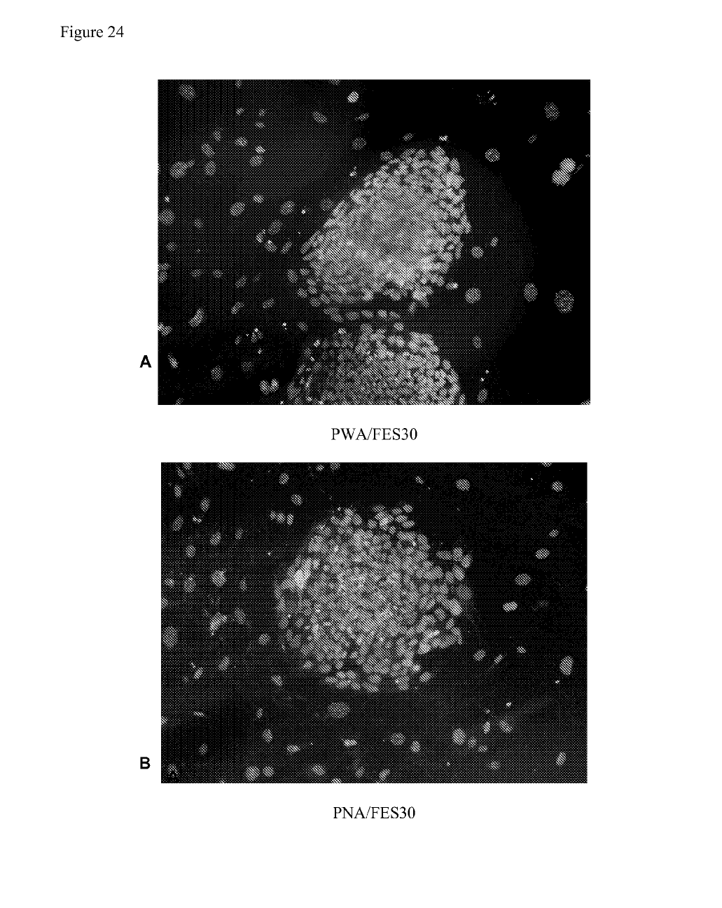

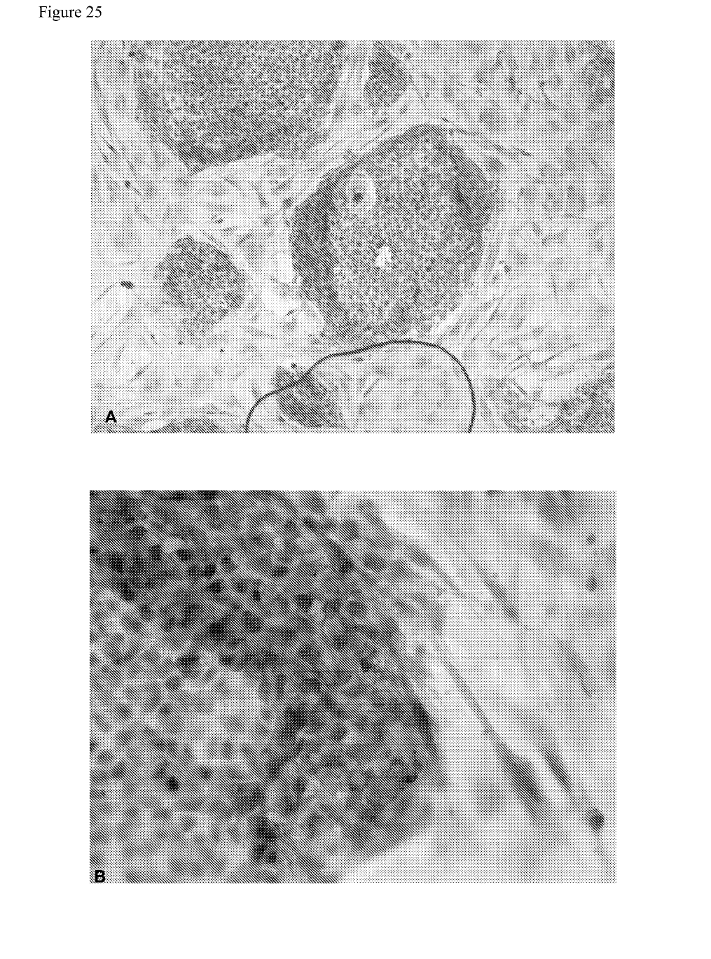

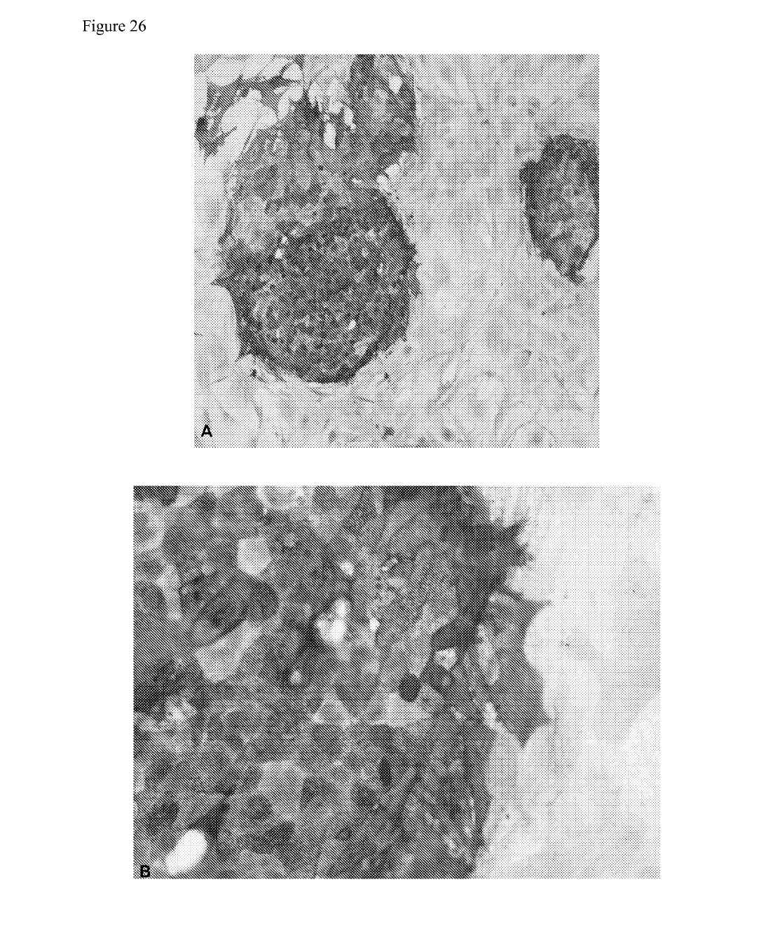

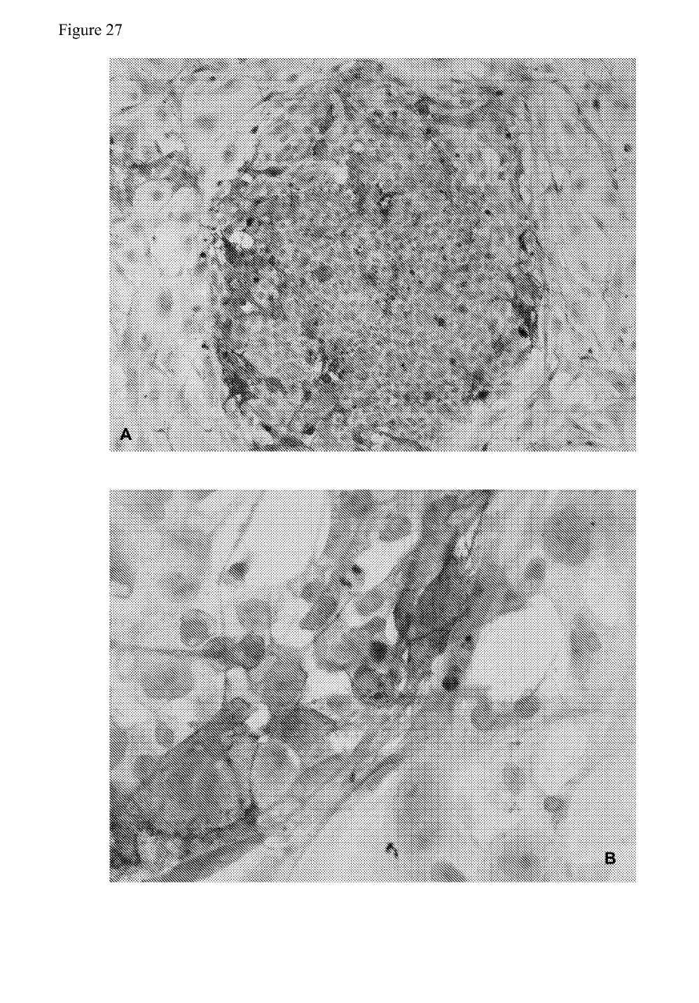

To identify differentiation stage specific N-glycan signals in sialylated N-glycan profiles of hESC, EB, and stage 3 differentiated cells (see Examples above), major signals specific to either the undifferentiated (FIG. 19) or differentiated cells (FIG. 20) were selected based on their relative abundances in the database of the four hESC lines, and the four EB and st.3 cell samples derived from the four hESC lines, respectively. The selected glycan signal groups, from where indifferent glycan signals have been removed, have reduced noise or background and less observation points, but have the resolving power. Such selected signal groups and their patterns in different sample types serve as a signature for the identification of, for example, 1) undifferentiated hESC (FIG. 19), 2) differentiated cells, preferentially their differentiation stage relative to hESC (FIG. 20), 3) differentiation lineage, such as the neuroectodermally enriched st.3 cells compared to the mixed cell population of EB (e.g. 1799), 4) glycan signals that are specific to hESC (e.g. 2953), 5) glycan signals that are specific to differentiated cells (e.g. 2644), or 6) glycan signals that have individual i.e. cell line specific variation (e.g. 1946 in cell line FES 22, 2133 in cell line FES 29, and 2222 in cell line FES 30). Moreover, glycan signals can be identified that do not change during hESC differentiation, including major glycans that can be considered as housekeeping glycans in hESC and their progeny (e.g. 1257, 1419, 1581, 1743, 1905 in FIG. 17.A, and 2076 in FIG. 17.B). Proposed glycan compositions and structure groups for the signals are presented in Table 43.

To further analyze the data and to find the major glycan signals associated in given hESC differentiation stage, two variables were calculated for the comparison of glycan signals in the N-glycan profile dataset described above, between two samples:

absolute difference A=(S2−S1), and 1.

relative difference R=A/S1, 2.

wherein S1 and S2 are relative abundances of a given glycan signal in samples 1 (the four EB samples) and 2 (the four st.3 cell samples), respectively.

When A and R were calculated for the glycan profile datasets of the two cell types, and the glycan signals thereafter sorted according to the values of A and R, the most significant differing glycan signals between the two samples could be identified. Among the fifty most abundant neutral N-glycan signals in the data (FIG. 17.A), the following five signals experienced the highest relative change R in the transition from EB to st.3 differentiated cells in the dataset of four EB and four st.3 cell samples: 1825 (R=5.8, corresponding to 6.8-fold increase), 1136 (R=1.4, corresponding to 2.4 fold increase), 1339 (R=0.9, corresponding to 1.9 fold increase), 2142 (R=0.87, corresponding to 87% decrease), and 2174 (R=0.56, corresponding to 56% decrease). Four of these signals corresponded to complex-type structures (Table 43), indicating that the major differing glycan structures were included in the complex-type glycan group. However, the majority of the other complex-type glycan signals in the dataset were not observed to differ as significantly between the two cell types (i.e. they did not have large values of A and/or R), indicating that the procedure was able to identify st.3 cell and EB associated glycan subgroups within the whole complex-type glycan group. The one signal corresponding to hybrid-type structures (1136) had the highest value of the absolute differences A among all the glycan signals in the neutral N-glycan profiles (A=0.48), indicating that also this signal had significance in the discrimination between the EB and st.3 cell samples in the studied dataset.

EB derived from the hESC line FES 30 were different in their overall N-glycan profiles compared to the other three EB samples (FIG. 17) and had the differentiation-specific glycan score value closer to the hESC samples (FIG. 5), correlating with the property of EB 30 having less differentiated cell types than the other three EB. This was also seen in distinct glycan signals, e.g. 2222 in FIG. 17.B.

### Example 9

### Schematic Concepts of Glycome Change and Mass Spectrometric Screening

Introduction to Glycomics

All human cell types have unique glycome—an entity of all glycans of the cell, present mainly on cell surface glycoproteins and glycolipids, including the SSEA and Tra glycan antigens. However, the whole spectrum of hESC glycan structures (the stem cell glycome) is still unknown. Glycans, the complex carbohydrate structures, are capable of great structural variation and their specific molecular structures carry diverse biological information.

### Example 10

Data Preparation

The mass data was normalized by dividing selected peaks with the total sum of the peak intensities of the corresponding spectra. Finally, normalized mass data from hESC, embryonic bodies, and stage 3 differentiated hESC was tabulated in Excel spread sheet and imported in Statistica 7.0 software (StatSoft).

Data Cleaning

Neutral and Acidic Glycans

In certain cases sample were divided into two tubes and MALDI was performed separately. In these cases data from the separate shots were combined and represented by their average intensity.

If all or almost all data values were zero, the corresponding mass was removed from the data set. For analyses requiring variance such as one way ANOVA and Factor analysis, further removal of masses were performed if all or almost all values were zeros in some subcategory.

### Example 11

ANOVA

One way ANOVA was performed to analyze basic statistics of the data. The means, standard deviations, box and whisker blots were screened to have an overall view of the data and to identify mass peaks with variation between different cell lines or differentiation stage. The one way ANOVA analysis was performed in Statistica with Fisher LSD post hoc analysis.

### Example 12

Factor Analysis

Factor analysis was employed in order to find “hidden” factors which would explain the variation within the mass distribution and their intensities. Moreover, by using factor analysis, the total variation could be explained with a smaller number of variables which simplifies the analysis.

The factor analysis (Principal component, Varimax normalised, Eigenvalues >1.0, factor loadings >0.62) indicated 7 to 8 main factors when explained variance >5% was considered as a cut off for a factor to be included into the model.

The 8 factors for acidic glycans comprised in the following masses:

F1: 1678, 1727, 1873, 1889, 1914, 2002, 2367, 2441, 2732, 2807, 2880, 3099 and 3172

F2: 1475, 1637, 1799, 2076, 2133, 2482, and 2714

F3: 2221, 2279, 2280, 2570, 2571, 2644, 2645, 2936, and 3098

F4: 1354, 1500, 1516, 1541, 1791,2010,2156,2230, 2246, and 2447

F5: 2011, 2321, and 2603

F6: 2254, 2528, 2544, 3025 and 3390

F7: 3024

F8: 2400 and 3170

The 7 factors for neutral glycans comprised in the following masses:

F1: 609, 771, 892, 933, 1054, 1095, 1216, 1378, 1540, 1702, 1743, 1809, 1955, 2028 and 2174

F2: 1460, 1485, 1606, 1622, 1647, 1704, 1850, 1866 and 2021

F3: 917, 1120, 1241, 1282, 1298, 1339, 1403, 1444, 1501, 1793, 1987 and 1996

F4: 1136, 1209, 1590, 2158, 2391 and 2466

F5: 730, 1031, 1565, 1825, 2117 and 2304

F6: 1257 and 1905

F7: 1784 and 2229

### Correlation Matrix, Neutral N-Glycan Fraction

Soluble HexNAc1-glycans H(4-9)N1 intercorrelate significantly. The correlation matrix reveals two subgroups: 1) H4N1, H5N1, and H6N1 comprising smaller soluble HexNAc1-glycans H(4-6)N1; and 2) H6N1, H7N1, H8N1, and H9N1 comprising larger soluble HexNAc1-glycans H(6-9)N1. H3N1 correlates most significantly with H4N1 but not with the other soluble HexNAc1-glycans.

The soluble HexNAc1-glycans further negatively correlate with low-mannose type N-glycans, most significantly with non-fucosylated low-mannose type N-glycans H2N2, H3N2, and H4N2; and with complex-type N-glycans with H═N terminal HexNAc composition feature, most significantly with H5N5F3.

The soluble HexNAc1-glycans further negatively correlate with complex-type N-glycans, most significantly with H5N4, H5N4F1, H6N5, and H6N5F1; and with high-mannose type N-glycans, most significantly with H8N2.

High-mannose type N-glycans H(6-8)N2 intercorrelate significantly; whereas H9N2 correlates significantly with glucosylated high-mannose type N-glycan H10N2; and H5N2 negatively correlates with the larger H(6-9)N2 glycans, most significantly with H9N2; and the fucosylated high-mannose type N-glycans H5N2F1 and H6N2F1 correlate significantly with the fucosylated low-mannose type N-glycans. Therefore, the correlation matrix reveals four differently regulated groups within the high-mannose type N-glycans: 1) H5N2, 2) H(6-8)N2, 3) H(9-10)N2, and 4) H(5-6)N2F1; groups 3) and 2) are preferentially expressed in hESC; and 1) and 4) in the differentiated cell types.

In the following analysis of the performed factor analyses, glycan signals were assigned into glycan structure classes as described in the present invention and coded by the following one letter-code: A=acidic, C=complex-type, H=hybrid-type, S=soluble HexNAc1-type, O=other types, L=low-mannose type, M=high-mannose type, N=monoantennary type, B=biantennary-size complex-type, R=larger than biantennary-size complex-type, F=fucosylated, E=complex-fucosylated i.e. containing more than one dHex residue, P=sulphated or phosphorylated, T=terminal HexNAc, wherein n(N)>n(H), Q=terminal HexNAc, wherein n(N)=n(H), X=terminal Hex in complex-type N-glycan, wherein n(H)>n(N)+1, A=acetylated, Y=containing N-glycolylneuraminic acid.

Factor Analysis, Neutral N-Glycan Fraction

Factor 1 reflects positive contribution of:
1) soluble HexNAc1-type glycans, preferably including H(4-9)N1, and
2) non-fucosylated low-mannose type N-glycans, preferably including H(2-4)N2;

and negative contribution of:
3) large high-mannose type N-glycans, preferably including H(7-8)N2,
4) neutral biantennary-size complex-type N-glycans, preferably including H5N4F(1-2),
5) neutral triantennary-size complex-type N-glycans, preferably including H6N5F(0-1), and
6) H1N2 low-mannose type N-glycans.

In a preferred embodiment of the present invention, Factor 1 reflects a switch between glycan groups Factor 1-1 and Factor 1-2; and glycan groups Factor 1-3, Factor 1-4, Factor 1-5, and Factor 1-6. In a further preferred embodiment, relative high expression of one or more of the first glycan groups is associated with relative low expression of the latter glycan groups, and vice versa. In another further preferred embodiment, the first glycan groups are associated with differentiated cells and the latter glycan groups are associated with hESC.

Positive contribution:

| | | | |
|---|---|---|---|
| H6N1 | S | 1216 | 0.86 |
| H7N1 | S | 1378 | 0.86 |
| H9N1 | S | 1702 | 0.85 |
| H8N1 | S | 1540 | 0.82 |
| H4N1 | S | 892 | 0.81 |
| H3N2 | L | 933 | 0.81 |
| H4N2 | L | 1095 | 0.78 |
| H5N1 | S | 1054 | 0.78 |
| H2N2 | L | 771 | 0.72 |

Negative contribution:

| | | | | | |
|---|---|---|---|---|---|
| H5N4F2 | C | B | E | 1955 | −0.83 |
| H1N2 | L | | | 609 | −0.79 |
| H6N5F1 | C | R | F | 2174 | −0.79 |
| H5N4F1 | C | B | F | 1809 | −0.78 |
| H8N2 | M | | | 1743 | −0.74 |
| H6N5 | C | R | | 2028 | −0.73 |
| H7N2 | M | | | 1581 | −0.66 |

Factor 2 reflects negative contribution of:
1) neutral complex-type N-glycans with N>H type non-reducing terminal HexNAc, preferably including H4N5, H4N5F3, or H3N4F1,
2) neutral complex-type N-glycans with N=H type non-reducing terminal HexNAc, preferably including H5N5(F0-1) or H4N4F1, and
3) neutral large hybrid-type N-glycans, preferably including H5N3(F0-1) or H6N3.

In a preferred embodiment of the present invention, Factor 2 reflects the relative amount of the glycan groups Factor 2-1, Factor 2-2, or Factor 2-3. In a further preferred embodiment, these glycan groups are associated with differentiated cells.

Negative contribution:

| | | | | | |
|---|---|---|---|---|---|
| H4N4F1 | C | F | Q | 1647 | −0.67 |
| H5N5F1 | C | F | Q | 2012 | −0.71 |
| H5N3 | H | | | 1460 | −0.77 |
| H6N3 | H | | | 1622 | −0.79 |
| H3N4F1 | C | F | T | 1485 | −0.80 |
| H5N3F1 | H | F | | 1606 | −0.81 |
| H5N5 | C | | Q | 1866 | −0.86 |
| H4N5 | C | | T | 1704 | −0.88 |
| H4N5F3 | C | E | T | 1850 | −0.90 |

Factor 3 reflects positive contribution of:
1) neutral small hybrid-type or monoantennary N-glycans, preferably including H4N3;

and negative contribution of:
2) neutral fucosylated monoantennary N-glycans, preferably including H(2-3)N2F1,
3) fucosylated low- and high-mannose type N-glycans, preferably including H(4-5)N2F1,
4) neutral complex-type N-glycans with N>H type non-reducing terminal HexNAc, preferably including H3N4 or H4N5F2, and
5) neutral complex-type N-glycans with N═H type non-reducing terminal HexNAc, preferably including H4N4 or H4N4F2.

In a preferred embodiment of the present invention, Factor 3 reflects a switch between glycan groups Factor 3-1 and glycan groups Factor 3-2, Factor 3-3, Factor 3-4, and Factor 3-5. In a further preferred embodiment, relative high expression of the first glycan group is associated with relative low expression of the latter glycan groups, and vice versa. In another further preferred embodiment, the first glycan group is associated with hESC and the latter glycan groups are associated with differentiated cells.

Positive contribution:

H4N3 H 1298 0,78

Negative contribution:

| | | | | | | |
|---|---|---|---|---|---|---|
| H4N2F1 | L | | F | | 1241 | −0.71 |
| H4N4F2 | C | | E | Q | 1793 | −0.72 |
| H3N4 | C | | | T | 1339 | −0.81 |
| H5N2F1 | M | | F | | 1403 | −0.81 |
| H4N4 | C | | | Q | 1501 | −0.82 |
| H4N5F2 | C | | E | T | 1996 | −0.86 |
| H2N3F1 | H | N | F | T | 1120 | −0.88 |
| H3N3F1 | H | N | F | | 1282 | −0.91 |

Factor 4 reflects positive contribution of:
1) neutral monoantennary or small hybrid-type N-glycans, preferably including H3N3 or H4N3F2, and
2) neutral complex-type N-glycans with N═H type non-reducing terminal HexNAc and complex fucosylation, preferably including H5N5F2.

In a preferred embodiment of the present invention, Factor 4 reflects the relative amount of the glycan groups Factor 4-1 and Factor 4-2. In a further preferred embodiment, these glycan groups are associated with differentiated cells.

Positive contribution:

| | | | | | | |
|---|---|---|---|---|---|---|
| H5N5F2 | C | | E | Q | 2158 | 0.82 |
| H3N3 | H | N | | | 1136 | 0.82 |
| H4N3F2 | H | | E | | 1590 | 0.67 |

Factor 5 reflects positive contribution of:
1) small soluble HexNAc1-type glycans, preferably including H3N1,
2) neutral complex-type N-glycans with N═H type non-reducing terminal HexNAc and complex fucosylation, preferably including H5N5F3, and
3) fucosylated high-mannose type N-glycans, preferably including H6N2F1.

In a preferred embodiment of the present invention, Factor 5 reflects the relative amount of the glycan groups Factor 5-1, Factor 5-2, and Factor 5-3. In a further preferred embodiment, these glycan groups are associated with differentiated cells.

Positive contribution:

| | | | | | |
|---|---|---|---|---|---|
| H5N5F3 | C | E | Q | 2304 | 0.85 |
| H6N2F1 | M | F | | 1565 | 0.79 |
| H3N1 | S | | | 730 | 0.77 |

Factor 6 essentially reflects the positive contribution of small high-mannose type N-glycans (Factor 6-1), preferentially including H5N2 (positive contribution: 0.69), and negative contribution of large high-mannose type N-glycans (Factor 6-2), preferentially including H9N2 (positive contribution: −0.80). In a preferred embodiment of the present invention, Factor 6 reflects a switch between these glycan groups, wherein relative increase in one group is reflected in relative decrease in the other group. In a further preferred embodiment, Factor 6-1 is associated with differentiated cells and Factor 6-2 is associated with hESC.

Factor Analysis, Acidic Neutral N-Glycan Fractions

Factors A1 and A2 are mainly composed of contribution of neutral glycan signals.

Factor A3 reflects positive contribution of:
1) sialylated complex-type N-glycans with N>H type non-reducing terminal HexNAc, preferably including S1H4N5F(1-2),
2) sialylated monoantennary-type N-glycans, preferably including S1H4N3F1, and
3) large high-mannose type N-glycans preferably including H6N2;

and negative contribution of:
4) sulphated or phosphorylated N-glycans, preferably including H3N4F1P1, S(0-2)H5N4F1P1, S(0-1)H5N4P1, H4N3P1, S1H4N3F1P1, H4N4P1, S1H5N4F3P1, H6N5F1P1, and H6N5F3P1; wherein P is preferably sulphate ester.

In a preferred embodiment of the present invention, Factor A3 reflects a switch between glycan groups Factor A3-1, Factor A3-2, and Factor A3-3; and glycan group Factor A3-4. In a further preferred embodiment, relative high expression of the first glycan group is associated with relative low expression of the latter glycan groups, and vice versa. In another further preferred embodiment, the first glycan group is associated with hESC and the latter glycan groups are associated with differentiated cells.

Positive contribution:

| | | | | | | | | |
|---|---|---|---|---|---|---|---|---|
| H6N2 | | | M | | | | 1419 | 0.87 |
| S1H4N5F1 | A | S1 | C | | F | T | 2117 | 0.71 |
| S1H4N3F1 | A | S1 | H | N | F | | 1711 | 0.65 |
| S1H4N5F2 | A | S1 | C | | E | T | 2263 | 0.60 |

Negative contribution:

| | | | | | | | | | |
|---|---|---|---|---|---|---|---|---|---|
| H3N4F1P1 | A | | C | | F | P | T | 1541 | −0.89 |
| S1H5N4F1P1 | A | S1 | C | B | F | P | | 2156 | −0.88 |
| H5N4F1P1 | A | | C | B | F | P | | 1865 | −0.86 |
| S1H5N4P1 | A | S1 | C | B | | P | | 2010 | −0.83 |
| H4N3P1 | A | | H | | | P | | 1354 | −0.83 |
| S1H4N3F1P1 | A | S1 | H | N | F | P | | 1791 | −0.78 |
| H4N4P1 | A | | C | | | P | Q | 1557 | −0.74 |
| S2H5N4F1P1 | A | S2 | C | B | F | P | | 2447 | −0.72 |
| S1H5N4F3P1 | A | S1 | C | B | E | P | | 2448 | −0.71 |
| H6N5F1P1 | A | | C | R | F | P | | 2230 | −0.70 |
| H5N4P1 | A | | C | B | | P | | 1719 | −0.66 |
| H6N5F3P1 | A | | C | R | E | P | | 2522 | −0.66 |

Factor A4 reflects positive contribution of:
1) sialylated and neutral complex-type biantennary-size N-glycans, preferably including S1H5N4F(0-1) and H5N4F1;

and negative contribution of:
2) small disialylated glycans, preferably including S2H(2-4)N2F1 and S2H(2-3)N3F1,
3) sialylated and neutral complex-type N-glycans with N═H type non-reducing terminal HexNAc, preferably including H5N5F3, S1H5N5, and H5N5F1P1,
4) fucosylated high-mannose type N-glycans, preferably including H6N2F1, and
5) sialylated and neutral complex-type N-glycans with N>H type non-reducing terminal HexNAc, preferably including S1H5N6F2 and H3N5F1.

In a preferred embodiment of the present invention, Factor A4 reflects a switch between glycan group Factor A4-1; and glycan groups Factor A4-2, Factor A4-3, Factor A4-4, and Factor A4-5. In a further preferred embodiment, relative high expression of the first glycan group is associated with relative low expression of the latter glycan groups, and vice versa. In another further preferred embodiment, the first glycan group is associated with hESC and the latter glycan groups are associated with differentiated cells.

Positive contribution:

| | | | | | | | | |
|---|---|---|---|---|---|---|---|---|
| S1H5N4F1 | A | S1 | C | B | F | | 2076 | 0.67 |
| S1H5N4 | A | S1 | C | B | | | 1930 | 0.63 |
| G1H5N4F1 | A | S1 | C | B | F | Y | 2092 | 0.56 |
| H5N4F1 | | | C | B | F | | 1809 | 0.50 |

Negative contribution:

| | | | | | | | | | |
|---|---|---|---|---|---|---|---|---|---|
| S2H3N2F1 | A | S2 | O | | F | | | 1637 | −0.90 |
| H5N5F3 | | | C | | E | | Q | 2304 | −0.89 |
| S2H2N2F1 | A | S2 | O | | F | | | 1475 | −0.87 |
| S2H4N2F1 | A | S2 | O | | F | | | 1799 | −0.85 |
| H6N2F1 | | | M | | F | | | 1565 | −0.77 |
| S1H5N6F2 | A | S1 | C | | E | | T | 2482 | −0.76 |
| H3N5F1 | | | C | | F | | T | 1688 | −0.74 |
| H5N5F1P1 | A | | C | | F | P | Q | 2068 | −0.73 |
| S1H5N5 | A | S1 | C | | | | Q | 2133 | −0.69 |
| S2H2N3F1 | A | S2 | O | | F | | | 1678 | −0.61 |
| S2H3N3F1 | A | S2 | H | N | F | | | 1840 | −0.57 |

Factor A5 reflects negative contribution of:
1) neutral fucosylated monoantennary or hybrid-type N-glycans, preferably including H(2-4)N3F1,
2) fucosylated low- and high-mannose type N-glycans, preferably including H(4-5)N2F1,
3) neutral complex-type N-glycans with N>H type non-reducing terminal HexNAc, preferably including H4N5F2 and H3N4, and
4) neutral complex-type N-glycans with N═H type non-reducing terminal HexNAc, preferably including S1H5N5F1A1, H4N4F2, and H4N4.

In a preferred embodiment of the present invention, Factor A5 reflects a switch in relative amounts of glycan groups Factor A5-1, Factor A5-2, Factor A5-3, and Factor A5-4. In a further preferred embodiment, these glycan groups are associated with differentiated cells.

Negative contribution:

| | | | | | | | | | |
|---|---|---|---|---|---|---|---|---|---|
| H2N3F1 | | | H | N | F | T | | 1120 | −0.85 |
| S1H7N5F1 | A | S1 | C | | F | X | | 2603 | −0.82 |
| H4N2F1 | | | L | | F | | | 1241 | −0.80 |
| H5N2F1 | | | M | | F | | | 1403 | −0.79 |
| H3N3F1 | | | H | N | F | | | 1282 | −0.78 |
| H2N4F1 | | | O | | F | T | | 1323 | −0.76 |
| H4N5F2 | | | C | | E | T | | 1996 | −0.75 |
| S1H5N5F1A1 | A | S1 | C | | F | Q | A | 2321 | −0.75 |
| H3N4 | | | C | | | T | | 1339 | −0.74 |
| H4N4F2 | | | C | | E | Q | | 1793 | −0.73 |
| H4N3F1 | | | H | | F | | | 1444 | −0.71 |
| H4N4 | | | C | | | Q | | 1501 | −0.70 |

Factor A7 reflects positive contribution of:
1) sialylated hybrid-type N-glycans, preferably including S1H5N3F(0-1) and H6N3,
2) small disialylated glycans, preferably including S2H2N3F1 and S2H4N3F1,
3) small high-mannose type N-glycans, preferably including H5N2,

and negative contribution of:
4) large monosialylated complex-type N-glycans, preferably including S1H7N6F1, S(1-2)H6N5F1, S1H8N7F1, and S1H7N6F3, and
5) large high-mannose type and glucosylated N-glycans, preferably including H9N2 and H(10-11)N2.

In a preferred embodiment of the present invention, Factor A7 reflects a switch between glycan groups Factor A7-1, Factor A7-2, and Factor A7-3; and glycan groups Factor A7-4 and Factor A7-5. In a further preferred embodiment, relative high expression of the first glycan group is associated with relative low expression of the latter glycan groups, and vice versa. In another further preferred embodiment, the first glycan group is associated with differentiated cells and the latter glycan groups are associated with hESC.

Positive contribution:

| | | | | | | | |
|---|---|---|---|---|---|---|---|
| S1H6N3 | A | S1 | H | | | 1889 | 0.89 |
| S1H5N3F1 | A | S1 | H | | F | 1873 | 0.80 |
| S1H5N3 | A | S1 | H | | | 1727 | 0.72 |
| S2H2N3F1 | A | S2 | O | | F | 1678 | 0.70 |
| S2H4N3F1 | A | S2 | H | N | F | 2002 | 0.64 |
| H5N2 | | | M | | | 1257 | 0.58 |

Negative contribution:

| | | | | | | | |
|---|---|---|---|---|---|---|---|
| S1H7N6F1 | A | S1 | C | R | F | 2807 | −0.75 |
| S1H6N5F1 | A | S1 | C | R | F | 2441 | −0.71 |
| S1H8N7F1 | A | S1 | C | R | F | 3172 | −0.70 |
| S1H7N6F3 | A | S1 | C | R | E | 3099 | −0.68 |
| H10N2 | | | M | G | | 2067 | −0.64 |
| S2H6N5F1 | A | S2 | C | R | F | 2732 | −0.62 |
| H9N2 | | | M | | | 1905 | −0.55 |
| H11N2 | | | M | G | | 2229 | −0.52 |

Factor A8 reflects positive contribution of:
1) complex-fucosylated complex-type N-glycans, preferably including S1H6N5F2 and S1H5N4F(2-3);

and negative contribution of:
2) multisialylated biantennary-size complex-type N-glycans, preferably including S2H5N4 and S2H5N5F1,
3) sialylated complex-type N-glycans with N═H type non-reducing terminal HexNAc, preferably including S(1-2)H6N6F1 and S(1-2)H5N5F1, and
4) O-acetylated sialylated N-glycans, preferably including G1H6N5F2A1 and G1H5N4F2A1, or S1H7N5F1A1 and S1H6N4F1A1.

In a preferred embodiment of the present invention, Factor A8 reflects a switch between glycan group Factor A8-1; and glycan groups Factor A8-2, Factor A8-3, and Factor A8-4. In a further preferred embodiment, relative high expression of one or more of the first glycan groups is associated with relative low expression of the latter glycan groups, and vice versa. In another further preferred embodiment, the first glycan group is associated with hESC and the latter glycan groups are associated with differentiated cells.

In a further preferred embodiment of the present invention, Factor A8 reflects a switch between N-glycan antenna sialylation (Factor A8-2) and fucosylation (Factor A8-1).

Positive contribution:

| | | | | | | | | |
|---|---|---|---|---|---|---|---|---|
| S1H6N5F2 | A | S1 | C | R | E | | 2587 | 0.65 |
| G1H5N4F2 | A | S1 | C | B | E | Y | 2238 | 0.60 |
| S1H5N4F2 | A | S1 | C | B | E | | 2222 | 0.60 |
| S1H5N4F3 | A | S1 | C | B | E | | 2368 | 0.57 |

Negative contribution:

| | | | | | | | | | |
|---|---|---|---|---|---|---|---|---|---|
| G1H6N5F2A1 | A | S1 | C | | E | | AY | 2645 | −0.90 |
| S2H6N6F1 | A | S2 | C | R | F | Q | | 2936 | −0.87 |
| S2H7N6F1 | A | S2 | C | R | F | | | 3098 | −0.87 |
| S1H6N6F1 | A | S1 | C | R | F | Q | | 2644 | −0.86 |
| S2H5N4 | A | S2 | C | B | | | | 2221 | −0.84 |
| H7N3 | | | H | | | | | 1784 | −0.80 |
| S2H5N5F1 | A | S2 | C | | F | Q | | 2570 | −0.77 |
| S1H5N5F1 | A | S1 | C | | F | Q | | 2279 | −0.76 |
| S1H5N5F3 | A | S1 | C | | E | Q | | 2571 | −0.69 |
| G1H5N4F2A1 | A | S1 | C | | E | | AY | 2280 | −0.60 |

The results of this analysis are gathered in Tables 50 and 51 for hESC-associated and differentiated cell-associated identified glycan structure groups, respectively.

### Example 13

Correlation Analysis

Pearson Correlation analysis was performed in Statistica and correlations >0.7 or <−0.7 were considered relevant (see Tables 30 and 31).

### Example 14

Discriminant Function Analysis of Neutral N-Glycans

The statistically significant mass intensities (p<0.099) shown in Table 25 were used in Forward Stepwise Discriminant Analysis. The tolerance was 0.010, F value of 1.0 was used instead of the default value one in order to increase the statistical significance of the model.

Results

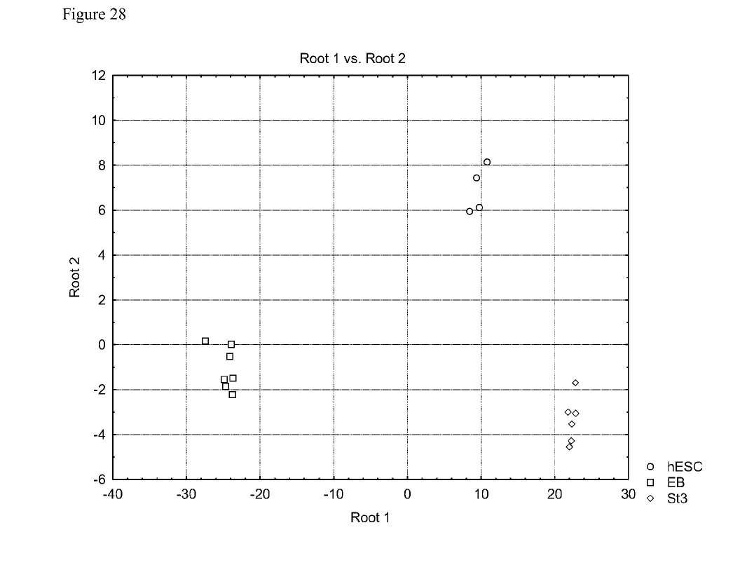

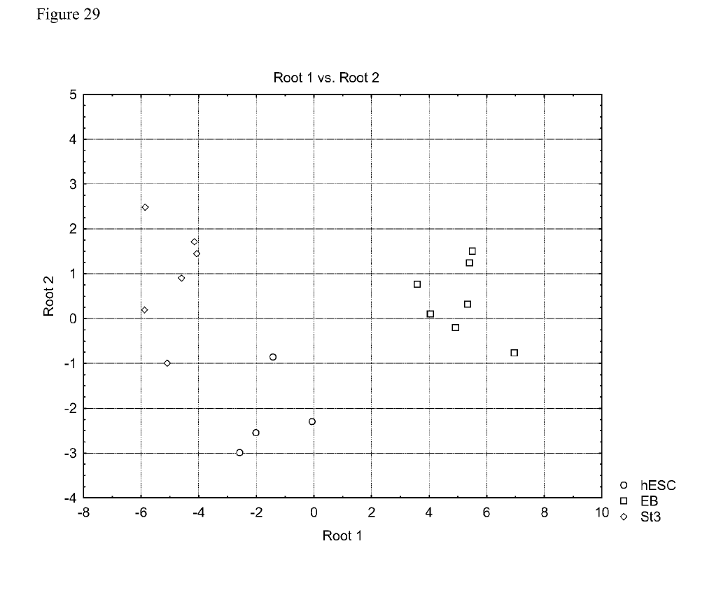

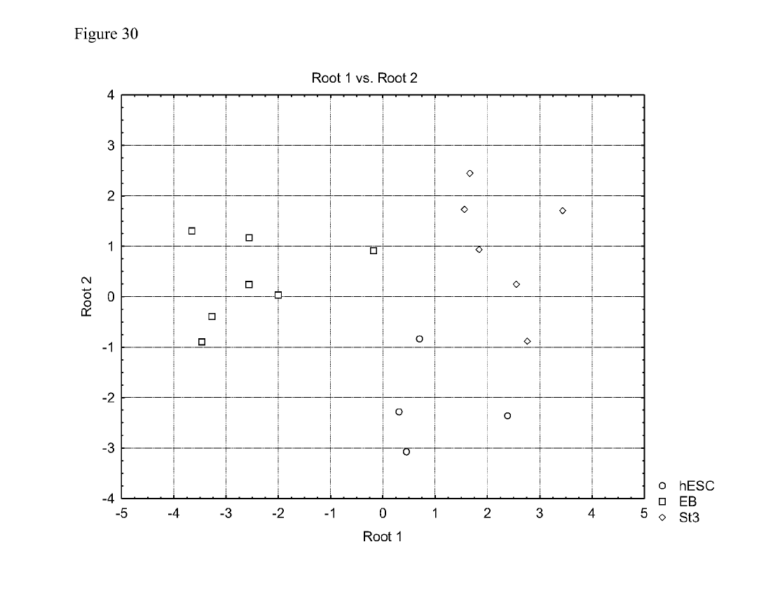

The Partial Wilks' Lambda in Table 32 indicates variables—in decreasing order of contribution—to the overall discrimination of the model. As highlighted below, the mass ‘2028’ is the most significant followed by 1825, 1054, 1419, 1688, 1905, 1095, 892, 1393 and mass ‘1540’ contributes the least to the overall discrimination. As the discrimination of the present model appeared to be high as shown in Root 1 and Root 2 (FIG. 28) and Eigenvalue of the Root 1 (543.7) compared to Root 2 (19.0) we performed removal of one mass by mass to limit the minimum number of masses to be able to discriminate undifferentiated human embryonic stem cells from embryoid bodies and stage 3 cells.

From Table 33 we notice that all p-levels are less than 0.05 meaning that all are significant. Furthermore this indicates that all centroids are well apart, i.e. the model discriminates very well between groups.

Canonical analysis Chi-squared test identified two statistically significant functions (canonical roots) which discriminate between hESC, EB and st3 and also to what percentage degree they discriminate.

From Table 34 we conclude that 543.7/(543.7+19.0)=96.7% of all discriminatory power is explained by first function, whereas the second function only explains 19.0/(543.7+19.0)=3.3%.

From Table 35 we identify the coefficients for each of the independent variables. The first discriminant function is weighted most heavily by the masses 1393, 1688 and 1540.

From Table 36, we identify the means of canonical variables. In this case we notice that the first discriminant function (Root 1) discriminates mostly between EB and st3.

The second discriminant function seems to distinguish mostly between hESC and EB/st3; however the magnitude of the discrimination is much smaller (3.3%).

In FIG. 28 this is represented more clearly. Root 1 is represented on the x-axis and Root 2 on the y-axis. From the figure we can see that the means are further differentiated on the x-axis and therefore we use Root 1 to determine the function.

Search for Minimal Discriminant Model

The original 10 masses identified from the first discrimination analysis was further subjected to one by one mass removal to identify the minimum masses still able to discriminate between groups. This was done by removing the smallest Partial Wilks' Lambda and performing above identified analysis. The second minimal set of masses to be able to discriminate comprises 5 masses shown in Table 37.

From Table 38 we conclude that 5.7/(5.7+1.8)=76% of all discriminatory power is explained by first function, whereas the second function explains 1.8/(5.7+1.8)=24%. From Table 38 it can be noticed that all p-levels are less than 0.05 meaning that all are significant. Furthermore this indicates that all centroids are well apart, i.e. the model discriminates very well between groups.

Model Function(s)

Based on the above raw coefficients the following models can be presented:

First Function (10 masses)

Y=7.58*2028−87.72*1393−20.37*1825−1.61*1419+26.91*1688−23.81*1540+2.47*1905+22.11*892−19.17*1095−3.66*1054+35.85

Y=differentiation degree

Second Minimal Function (5 masses)

Y=−2.97*892+4.94*1540−1.03*1905+16.50*1393−11.73*1688+15.56

First Minimal Function (4 masses)

Y=2.72*892−3.36*1540+0.64*1905+3.31*1688−10.62

### Example 15

Factor Analysis for Neutral and Acidic Glycans

Factor analysis was performed for combined data set for neutral and acidic glycans as described above. 8 factors were found which had explained more than 5% of total variance (Table 39).

### Example 16

Discriminant Analysis for Acidic Glycans

Discriminant analysis was performed as described above using Statistica General Discriminant Analysis module with the following parameters

Parameters: F to enter=5 and remove=2.0, and tolerance=0.010

### Example 17

Discriminant Analysis for Neutral and Acidic Glycans

Discriminant analysis was performed as described above using Statistica General Discriminant Analysis module with the following parameters

Parameters: F to enter and remove=1.0

p-value>0.05

### Example 18

FACS and immunohistochemical analysis of embryonic stem cells

Immunohistochemical staining of stem cells. Immunohistochemical studies of embryonic stem cells (in culture)(GF series of stainings). hESC were cultured as described in the Examples, fixed and after rinsing with PBS the stem cell cultures/sections were incubated in 3% highly purified BSA in PBS for 30 minutes at RT to block nonspecific binding sites. Primary antibodies (GF279, 288, 287, 284, 285, 283,286,290 and 289) were diluted (1:10) in PBS containing 1% BSA-PBS and incubated 1 hour at RT. Other antibodies indicated in the Tables were used similarily. After rinsing three times with PBS, the sections were incubated with biotinylated rabbit anti-mouse, secondary antibody (Zymed Laboratories, San Francisco, Calif., USA) in PBS for 30 minutes at RT, rinsed in PBS and incubated with peroxidase conjugated streptavidin (Zymed Laboratories) diluted in PBS. The sections were finally developed with AEC substrate (3-amino-9-ethyl carbazole; Lab Vision Corporation, Fremont, Calif., USA). After rinsing with water counterstaining was performed with Mayer's hemalum solution.

Antibodies, their antigens/epitopes and codes used in the immunostainings. Table 19 shows antibody binding to purified glycosphingolipid fractions from small amounts of cells (corresponding to hundreds of thousands of cells). The binding was analysed by TLC overlay assay using radiolabelled antibodies. The positive signals indicate presence of substantial amounts of the glycolipids and minus no signal due to too low amount for analysis.

Flow cytometry. Flow cytometric analysis of lectin binding was used to study the cell surface carbohydrate expression of hESC. The cells were washed with PBS. The cells were harvested into single cell suspensions by 0.02% Versene solution (pH7.4). Detached cells were centrifuged at 1100 g for five minutes at room temperature. Cell pellet was washed twice with 1% HSA-PBS, centrifuged at 1100 g and resuspended in 1% HSA-PBS. Cells were placed in conical tubes in aliquots of approximately 100000 cells each. Cell aliquots were incubated with one of the FITC labelled lectin for 30 minutes +4 C. After incubation cells were washed with 1% HSA-PBS, centrifuged and resuspended in 1% HSA-PBS. Untreated cells were used as controls. Lectin binding was detected by flow cytometry (FACSCalibur, Becton Dickinson).

In antibody analysis primary antibodies were incubated with suitable dilution based on recommendation of the producer for 30 minutes at +4 C and washed once with 0.3% HSA-PBS before secondary antibody detection with FITC secondary antibody for 30 minutes at +4 C in the dark. As a negative control cells were incubated without primary antibody and otherwise treated similar to labelled cells. Cells were analysed with BD FACS Calibur (Becton Dickinson). Results were analysed with Cell Quest Pro software (Becton Dickinson).

Fluorecently labeled lectins were from EY Laboratories (USA) or Vector Laboratories (UK). Antibody origin and codes are indicated in Table 20.

Results from FACS Analysis

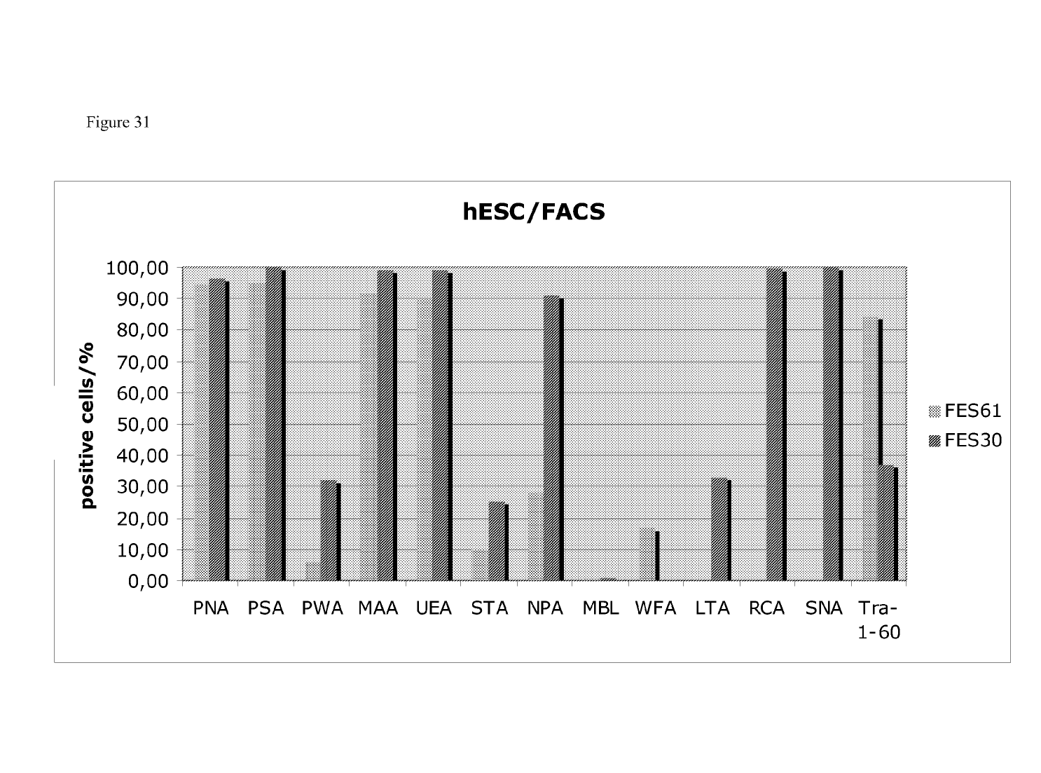

The lectin labelling results are present in Table 45 and FIGS. 31 and 18 from separate experiment for comparision. The symbol + indicates labelling majority of cell, ± indicates labelling of substantial subpopulation and (±) indicates weak labelling or labelling of minor cell population/few individual cells.

The antibody labelling results are present in Tables 46-8 and FIG. 32 with comparison to immunohistochemistry (immuno) results. The negativity—indicates negative or low labelling of less than 10% of cells when labelling with the specific antibody clone (defined in Table 20). The four most effective binders (for antigens H type I, H type I, type I LacNAc (Lewis c) and globotriose) were indicated with + in FACS Tables 46-47. These antibodies are especially preferred for recognition of the glycans under FACS conditions.

It is further realized that part of the structures indicated to be present can be recognized with other antibodies specific for the correct elongated glycan epitopes (e.g. Lewis x structures). The binding of LTA lectin verified the structural analysis of Lewis x on the specific N-glycan structures and the invention is specifically directed to known regents for the recognition of the N-glycan linked Lex according ot the invention. The schistosoma directed LacdiNAc specific antibodies form Leiden university appear not to be very effective in the recognition of the preferred N-glycan linked LacdiNAcs.

The comparision of the immunohistochemistry and FACS results indicates that the due to technical reasons FACS may be as effective for recognition of glycans observable by immunohistochemistry. The immunohistochemistry further reveals structures present in a few cells observable as very weak signals in FACS.

### Example 24

Gene Expression and Glycome Profiling of Human Embryonic Stem Cells

Results and Discussion

Obtaining of the gene expression data from the hESC lines FES 21, 22, 29, and 30 has been described (Skottman et al., 2005) and the present data was produced essentially similarily. The results of the gene expression profiling analysis with regard to a selection of potentially glycan-processing and accessory enzymes are presented in Table 49, where gene expression is both qualitatively determined as being present (P) or absent (A) and quantitatively measured in comparison to embryoid bodies (EB) derived from the same cell lines.

Fucosyltransferase expression levels. Three fucosyltransferase transcripts were detected in hESC: FUT1 (α1,2-fucosyltransferase; increased in all FES cell lines), FUT4 (α1,3-fucosyltransferase IV; increased in all FES cell lines), and FUT8 (N-glycan core α1,6-fucosyltransferase). The data supports the analysis of the presence of the preferred fucosylated structures in the non-differentiated stem cells.

Hexosaminyltransferase expression levels. The following transcripts in the selection of Table 49 were detected in hESC: MGAT3, MGAT2 (increased in three FES cell lines), MGAT1, GNT4b, β3GlcNAc-T5, β3GlcNAc-T7, β3GlcNAc-T4 (present in two FES cell lines), β6GlcNAcT (increased in one FES cell line), iβ3GlcNAcT, globosideT, and α4GlcNAcT (present in two FES cell lines).

Other gene expression levels. The following transcripts in the selection of Table 49 were detected in hESC: AER1 (increased in all FES cell lines), AGO61, β3GALT3, MAN1C1, and LGALS3.

In addition to fucosyltransferases I (FUT1), IV (FUT4), and VIII (FUT8), the expression of fucosyltransferase II (FUT2) was also detected in the hESC samples according to probe with the Affymetric code 210608_s_at. The expression was detected as “present” in hESC, but not significantly overexpressed compared to the embryoid bodies.

The product of the FUT2 gene is responsible for the synthesis of Fucα2Gal sequences, more preferably Fucα2Galβ3HexNAc, wherein HexNAc is either GlcNAc or GalNAc. According to the present invention, this gene product preferably fucosylates glycoconjugates in hESC specifically forming Fucα2Gal sequences (H antigens), more preferably Fucα2Galβ3GlcNAcβ (H type 1), Fucα2Galβ3GalNAcα (H type 3), and/or Fucα2Galβ3GalNAcβ (H type 4, Globo H) in hESC glycoconjugates including glycosphingolipid and glycoprotein glycans as described in the present invention.

Tables

TABLE 1
Neutral N-glycan difference analysis.

| | | | |
|---|---|---|---|
| composition¹⁾ | m/z²⁾ | class³⁾ | fold⁴⁾ |

+++ hESC⁵⁾

| | | | |
|---|---|---|---|
| H1N2 | 609 | M | 13.88 |
| H6N5F1 | 2174 | C | 3.33 |
| H6N5 | 2028 | C | 3.10 |
| H5N4F1 | 1809 | C | 2.26 |
| H5N4F2 | 1955 | C | 2.26 |

++ hESC

| | | | |
|---|---|---|---|
| H4N5F3 | 2142 | C | 1.61 |
| H5N4F3 | 2101 | C | 1.56 |

+ hESC

| | | | |
|---|---|---|---|
| H11N2 | 2229 | M | 1.49 |
| H5N4 | 1663 | C | 1.32 |
| H10N2 | 2067 | M | 1.28 |
| H8N2 | 1743 | M | 1.23 |
| H9N2 | 1905 | M | 1.16 |
| H4N3F1 | 1444 | H | 1.13 |
| H7N2 | 1581 | M | 1.10 |
| H4N3 | 1298 | H | 1.08 |
| H4N4F1 | 1647 | C | 1.08 |
| H6N2 | 1419 | M | 1.04 |
| H4N5 | 1704 | C | 1.02 |

+ Differentiated

| | | | |
|---|---|---|---|
| H5N3F1 | 1606 | H | 0.98 |
| H3N4F1 | 1485 | C | 0.92 |
| H5N3 | 1460 | H | 0.89 |
| H6N3F1 | 1768 | H | 0.81 |
| H5N2 | 1257 | M | 0.76 |
| H4N2 | 1095 | M | 0.73 |
| H6N3 | 1622 | H | 0.72 |
| H5N5F1 | 2012 | C | 0.66 |

++ Differentiated

| | | | |
|---|---|---|---|
| H5N5 | 1866 | C | 0.65 |
| H3N3 | 1136 | H | 0.59 |
| H3N2 | 933 | M | 0.58 |
| H3N3F1 | 1282 | H | 0.57 |
| H4N2F1 | 1241 | M | 0.57 |

+++ Differentiated

| | | | |
|---|---|---|---|
| H3N2F1 | 1079 | M | 0.46 |
| H4N3F2 | 1590 | H | 0.42 |
| H3N5F1 | 1688 | C | 0.31 |
| H5N2F1 | 1403 | M | 0.31 |
| N2N2F1 | 917 | M | 0.29 |
| H4N5F1 | 1850 | C | 0.24 |
| H2N4F1 | 1323 | A | 0.24 |
| H2N2 | 771 | M | 0.24 |
| H4N4 | 1501 | C | 0.19 |
| H4N4F2 | 1793 | C | 0.16 |
| H4N5F2 | 1996 | C | 0.15 |
| H6N4 | 1825 | C | 0.13 |
| H3N5 | 1542 | C | 0.12 |
| H6N2F1 | 1565 | M | 0 |
| H2N3F1 | 1120 | H | 0 |
| H7N4 | 1987 | C | 0 |

¹⁾Proposed composition wherein the monosaccharide symbols are: H, Hex; N, HexNAc; F, dHex.
²⁾Calculated m/z for [M + Na]+ ion rounded down to next integer.
³⁾N-glycan class symbols are: H, hybrid-type or monoantennary; C, complex-type; O, other type; F, fucosylated; E, complex-fucosylated, wherein at least one fucose residue is α1,2-, α1,3- or α1,4-linked.
⁴⁾‘fold’ is calculated as the relation of glycan signal intensities in hESC compared to differentiated cell types (hESC and St.3); 0, not detected in hESC.
⁵⁾Association with differentiation type based on fold calculation: + low association, ++ substantial association, +++ high association.

TABLE 2
Sialylated N-glycan difference analysis.

| | | | |
|---|---|---|---|
| composition¹⁾ | m/z²⁾ | class³⁾ | fold⁴⁾ |

+++ hESC⁵⁾

| | | | |
|---|---|---|---|
| S1H7N6F2 | 2953 | CE | ∞ |
| S1H8N7F1 | 3172 | CF | ∞ |
| S1H7N6F3 | 3099 | CE | 15.67 |
| S2H4N5F1 | 2408 | CF | 5.07 |
| G2H5N4 | 2253 | C | 4.56 |
| G1H5N4 | 1946 | C | 4.50 |
| S1H5N4F2 | 2222 | CE | 3.81 |
| S2H6N4 | 2383 | C | 3.51 |
| G1H5N4F1 | 2092 | CF | 3.13 |
| S1H6N5F2 | 2587 | CE | 2.94 |
| S1G1H5N4 | 2237 | C | 2.68 |
| S1H6N4F2 | 2384 | CE | 2.42 |
| S1H5N4F3 | 2368 | CE | 2.02 |

++ hESC

| | | | |
|---|---|---|---|
| S2H5N4F1 | 2367 | CF | 1.83 |
| S3H6N5 | 2878 | C | 1.82 |
| S2H6N5F1 | 2732 | CF | 1.80 |
| S1H4N5F2 | 2263 | CE | 1.59 |

+ hESC

| | | | |
|---|---|---|---|
| S2H6N5F2 | 2879 | CE | 1.49 |
| S1H7N6F1 | 2807 | CF | 1.39 |
| S1H6N5F1 | 2441 | CF | 1.20 |
| S1H5N4 | 1930 | C | 1.17 |
| S1H5N4F1 | 2076 | CF | 1.14 |
| S1H6N5F3 | 2733 | CE | 1.11 |
| S1H6N5 | 2295 | C | 1.06 |
| S1H6N4F1 | 2238 | CF | 1.03 |

+ Differentiated

| | | | |
|---|---|---|---|
| S2H7N6F1 | 3098 | CF | 0.75 |
| S1H5N5F2 | 2425 | CE | 0.71 |
| S2H5N4 | 2221 | C | 0.70 |
| S1H4N3F1 | 1711 | HF | 0.69 |
| S1H4N3 | 1565 | H | 0.68 |

++ Diff

| | | | |
|---|---|---|---|
| S1H4N5F1 | 2117 | CF | 0.66 |
| S2H5N3F1 | 2164 | HF | 0.56 |
| S1H5N3 | 1727 | H | 0.52 |

+++ Diff

| | | | |
|---|---|---|---|
| S1H6N3 | 1889 | H | 0.47 |
| S2H3N3F1 | 1840 | OF | 0.30 |
| S1H4N4F1 | 1914 | CF | 0.29 |
| S1H5N3F1 | 1873 | HF | 0.28 |
| S2H2N3F1 | 1678 | OF | 0.27 |
| S2H4N3F1 | 2002 | OF | 0.20 |
| S2H5N5F1 | 2570 | CF | 0.19 |
| S1H5N5F1 | 2279 | CF | 0.17 |
| S1H5N5 | 2133 | C | 0.15 |
| S1H6N4F1Ac | 2280 | CF | 0.13 |
| S1H6N3F1 | 2035 | HF | 0 |
| S1H6N6F1 | 2644 | CF | 0 |
| S1H5N6F2 | 2482 | CE | 0 |
| S1H7N5F1Ac | 2645 | CF | 0 |
| S1H5N5F3 | 2571 | CE | 0 |

¹⁾Proposed composition wherein the monosaccharide symbols are: S, NeuAc; G, NeuGc, H, Hex; N, HexNAc; F, dHex; Ac, acetyl ester.
²⁾Calculated m/z for [M − H]− ion rounded down to next integer.
³⁾N-glycan class symbols are: H, hybrid-type or monoantennary; C, complex-type; O, other type; F, fucosylated; E, complex-fucosylated, wherein at least one fucose residue is α1,2-, α1,3- or α1,4-linked.
⁴⁾‘fold’ is calculated as the relation of glycan signal intensities in hESC compared to differentiated cell types (hESC and St.3); ∞, not detected in differentiated cells; 0, not detected in hESC.
⁵⁾Association with differentiation type based on fold calculation: + low association, ++ substantial association, +++ high association.

TABLE 3
N-glycan structural feature analysis based on proposed monosaccharide compositions of
four hESC lines FES 21, FES 22, FES 29, and FES 30.

| | | | | | FES 21* | FES 22 | FES 29 | FES 30 | EB | st.3 |
|---|---|---|---|---|---|---|---|---|---|---|
| Neutral | A | N = 2 and 5 ≦ H ≦ 10 | | high-mannose type | 84^{#} | 73 | 79 | 79 | 73 | 72 |
| [symbol] | B | N = 2 and 1 ≦ H ≦ 4 | | low-mannose type | 5 | 11 | 7 | 8 | 12 | 12 |
| | C | N = 3 and H ≧ 2 | | hybrid/monoantennary | 3 | 7 | 3 | 3 | 5 | 6 |
| | D | N ≧ 4 and H ≧ 3 | | complex-type | 6 | 9 | 10 | 10 | 8 | 8 |
| | E | other types | | | 2 | 0 | 1 | 0 | 2 | 2 |
| | N ≧ 3 | F | F ≧ 1 | fucosylation | 8 | 11 | 10 | 10 | 14 | 15 |
| | | G | F ≧ 2 | complex fucosylation | 1 | 0 | 2 | 2 | 2 | 2 |
| | | H^{§} | N > H ≧ 2 | terminal N (N > H) | 1 | 2 | 1 | 1 | 3 | 3 |
| | | I | N = H ≧ 5 | terminal N (N = H) | 0 | 2 | 0 | 0 | 1 | 1 |
| Sialylated | J | N = 3 and H ≧ 3 | | hybrid/monoantennary | 8 | 2 | 5 | 9 | 13 | 14 |
| [symbol] | K | N ≧ 4 and H ≧ 3 | | complex-type | 91 | 98 | 94 | 90 | 83 | 77 |
| | L | other types | | | 1 | 0 | 1 | 1 | 4 | 9 |
| | N ≧ 3 | M | F ≧ 1 | fucosylation | 85 | 96 | 75 | 78 | 83 | 86 |
| | | N | F ≧ 2 | complex fucosylation | 24 | 34 | 23 | 19 | 12 | 11 |
| | | O | N > H ≧ 3 | terminal N (N > H) | 10 | 8 | 6 | 5 | 10 | 10 |
| | | P | N = H ≧ 5 | terminal N (N = H) | 3 | 4 | 4 | 2 | 14 | 20 |

The numbers refer to percentage from either neutral (A-E) or acidic (J-L) N-glycan pools, or from subfractions of hybrid/monoantenary and complex-type N-glycans (N ≧ 3, F-I and M-P).
EB 29 and EB 30: embryoid bodies derived from hESC lines FES 29 and FES 30, respectively;
st.3 29: stage 3 differentiated cells derived from hESC line FES 29.
H: hexose;
N: N-acetylhexosamine;
F: deoxyhexose. [symbol]
indicates data missing or illegible when filed

TABLE 4

| Proposed composition | m/z | hESC | EB | st.3 | hEF | mEF | BM MSC | OB | CB MSC | AC | CB MNC | CD 34+ | CD 133+ | LIN− | CD 8− |
|---|---|---|---|---|---|---|---|---|---|---|---|---|---|---|---|
| Hex₅₋₉HexNAc₂ | | | | | | | | | | | | | | | |
| (including high-mannose type | | | | | | | | | | | | | | | |
| N-glycans) | | | | | | | | | | | | | | | |
| Hex5HexNAc2 | 1257 | + | + | + | + | + | + | + | + | + | + | + | + | + | + |
| Hex6HexNAc2 | 1419 | + | + | + | + | + | + | + | + | + | + | + | + | + | + |
| Hex7HexNAc2 | 1581 | + | + | + | + | + | + | + | + | + | + | + | + | + | + |
| Hex8HexNAc2 | 1743 | + | + | + | + | + | + | + | + | + | + | + | + | + | + |
| Hex9HexNAc2 | 1905 | + | + | + | + | + | + | + | + | + | + | + | + | + | + |
| Hex₁₋₄HexNAc₂dHex₀₋₁ | | | | | | | | | | | | | | | |
| (including low-mannose type | | | | | | | | | | | | | | | |
| N-glycans) | | | | | | | | | | | | | | | |
| HexHexNAc2 | 609 | + | + | | | | | + | + | + | + | | + | | + |
| HexHexNAc2dHex | 755 | | | | | | | | | + | + | + | + | | + |
| Hex2HexNAc2 | 771 | + | + | + | + | + | + | + | + | + | + | + | + | + | + |
| Hex2HexNAc2dHex | 917 | + | + | + | + | + | + | + | + | + | + | + | + | + | + |
| Hex3HexNAc2 | 933 | + | + | + | + | + | + | + | + | + | + | + | + | + | + |
| Hex3HexNAc2dHex | 1079 | + | + | + | + | + | + | + | + | + | + | + | + | + | + |
| Hex4HexNAc2 | 1095 | + | + | + | + | + | + | + | + | + | + | + | + | + | + |
| Hex4HexNAc2dHex | 1241 | + | + | + | + | + | + | + | + | + | + | + | + | + | + |
| Hex₁₀₋₁₂HexNAc₂ | | | | | | | | | | | | | | | |
| (including glucosylated high- | | | | | | | | | | | | | | | |
| mannose type N-glycans) | | | | | | | | | | | | | | | |
| Hex10HexNAc2 | 2067 | + | + | + | + | + | + | + | + | + | + | + | + | + | + |
| Hex11HexNAc2 | 2229 | + | + | + | + | + | + | + | + | + | + | | | | + |
| Hex12HexNAc2 | 2391 | + | + | | + | + | + | + | + | + | + | | | | + |
| Hex₅₋₉HexNAc₂dHex₁ | | | | | | | | | | | | | | | |
| (including glucosylated high- | | | | | | | | | | | | | | | |
| mannose type N-glycans) | | | | | | | | | | | | | | | |
| Hex5HexNAc2dHex | 1403 | + | + | + | + | + | + | + | + | + | + | + | + | + | + |
| Hex6HexNAc2dHex | 1565 | | + | | + | | + | + | + | + | + | | + | + | + |
| Hex7HexNAc2dHex | 1727 | | | | | | | | | | + | | | | |
| Hex₁₋₉HexNAc₁ | | | | | | | | | | | | | | | |
| (including soluble glycans) | | | | | | | | | | | | | | | |
| Hex2HexNAc | 568 | | | | + | + | + | + | + | + | | + | | | |
| Hex3HexNAc | 730 | | | | + | + | + | + | + | + | + | + | + | | |
| Hex4HexNAc | 892 | + | + | + | + | + | + | + | + | + | + | + | + | + | + |
| Hex5HexNAc | 1054 | + | + | + | + | + | + | + | + | + | + | + | + | + | + |
| Hex6HexNAc | 1216 | + | + | + | + | + | + | + | + | + | + | + | + | + | + |
| Hex7HexNAc | 1378 | + | + | + | + | + | + | + | + | + | + | + | + | + | + |
| Hex8HexNAc | 1540 | + | + | + | + | + | + | + | + | + | + | + | + | + | |
| Hex9HexNAc | 1702 | + | + | + | + | + | + | | + | | + | + | | + | |
| HexNAc = 3 and Hex ≧ 2 | | | | | | | | | | | | | | | |
| (including hybrid-type and | | | | | | | | | | | | | | | |
| monoantennary N-glycans) | | | | | | | | | | | | | | | |
| Hex2HexNAc3 | 974 | | | | | | | + | + | + | | | | | |
| Hex2HexNAc3dHex | 1120 | | + | | | | + | + | + | + | + | + | + | | + |
| Hex3HexNAc3 | 1136 | + | + | + | + | + | + | + | + | + | + | + | + | + | + |
| Hex2HexNAc3dHex2 | 1266 | | | | | | | | + | | | | | | |
| Hex3HexNAc3dHex | 1282 | + | + | + | + | + | + | + | + | + | + | + | + | + | + |
| Hex4HexNAc3 | 1298 | + | + | + | + | + | + | + | + | + | + | + | + | + | + |
| Hex3HexNAc3dHex2 | 1428 | | | | | | | + | | + | + | + | + | | + |
| Hex4HexNAc3dHex | 1444 | + | + | + | + | + | + | + | + | + | + | + | + | + | + |
| Hex5HexNAc3 | 1460 | + | + | + | + | + | + | + | + | + | + | + | + | + | + |
| Hex4HexNAc3dHex2 | 1590 | + | | | | | + | + | + | + | + | + | + | | + |
| Hex5HexNAc3dHex | 1606 | + | + | + | + | + | + | + | + | + | + | + | + | + | + |
| Hex6HexNAc3 | 1622 | + | + | + | + | + | + | + | + | + | + | + | + | + | + |
| Hex5HexNAc3dHex2 | 1752 | | | | | | + | + | + | + | | | | | |
| Hex6HexNAc3dHex | 1768 | + | + | | | + | + | + | + | + | | | + | | + |
| Hex7HexNAc3 | 1784 | | | + | + | + | + | + | + | + | | | | | |
| Hex8HexNAc3 | 1946 | | | | + | + | | | | | | | | | |
| HexNAc ≧ 4 and Hex ≧ 3 | | | | | | | | | | | | | | | |
| (including complex-type N- | | | | | | | | | | | | | | | |
| glycans) | | | | | | | | | | | | | | | |
| Hex3HexNAc4 | 1339 | | | | + | + | + | + | + | + | + | | | + | |
| Hex3HexNAc4dHex | 1485 | + | + | + | + | + | + | + | + | + | + | + | + | + | + |
| Hex4HexNAc4 | 1501 | + | | | + | + | + | + | + | + | | + | | + | + |
| Hex3HexNAc5 | 1542 | + | + | + | + | + | | | | + | + | | | + | |
| Hex4HexNAc4dHex | 1647 | + | + | + | + | + | + | + | + | + | + | + | + | + | + |
| Hex5HexNAc4 | 1663 | + | + | + | + | + | + | + | + | + | + | + | + | + | + |
| Hex3HexNAc5dHex | 1688 | + | + | + | + | + | + | + | + | + | + | + | + | + | + |
| Hex4HexNAx5 | 1704 | + | + | + | + | + | | + | + | + | + | | + | + | + |
| Hex4HexNAc4dHex2 | 1793 | + | | | + | | | | + | + | + | + | + | | + |
| Hex5HexNAc4dHex | 1809 | + | + | + | + | + | + | + | + | + | + | + | + | + | + |
| Hex6HexNAc4 | 1825 | + | | + | + | + | + | + | + | + | + | | | + | + |
| Hex4HexNAc5dHex | 1850 | + | + | + | | | + | | + | + | + | | | | |
| Hex5HexNAc5 | 1866 | + | + | + | + | + | + | + | + | + | + | | | + | + |
| Hex3HexNAc6dHex | 1891 | | | | | | | + | + | + | + | | | + | |
| Hex5HexNAc4dHex2 | 1955 | + | + | | + | | + | + | + | + | + | + | + | | + |
| Hex6HexNAc4dHex | 1971 | | + | | + | + | + | + | + | + | | | | | + |
| Hex7HexNAc4 | 1987 | | | | + | + | + | + | + | + | | | | | + |
| Hex4HexNAc5dHex2 | 1996 | + | + | | + | | + | + | + | + | | | | | |
| Hex5HexNAc5dHex | 2012 | + | + | + | + | | + | + | + | + | | | | | |
| Hex6HexNAc5 | 2028 | + | + | + | + | + | + | + | + | + | + | | | | + |
| Hex5HexNAc4dHex3 | 2101 | + | + | | + | | + | + | + | + | + | + | + | | + |
| Hex6HexNAc4dHex2 | 2117 | | | | | | + | + | | | | | | | |
| Hex7HexNAc4dHex | 2133 | | | | + | + | | + | | | | | | | + |
| Hex4HexNAc5dHex3 | 2142 | + | + | | + | + | + | + | | | | | | | + |
| Hex8HexNAc4 | 2149 | | | | + | + | + | + | | | | | | + | |
| Hex5HexNAc5dHex2 | 2158 | | + | | | | | + | + | + | | | | | |
| Hex6HexNAc5dHex | 2174 | + | + | + | + | | + | + | + | + | | + | | | + |
| Hex7HexNAc5 | 2190 | | | | + | + | | | | | | | | | |
| Hex6HexNAc6 | 2231 | | | | | | | + | | + | | | | | |
| Hex7HexNAc4dHex2 | 2279 | | | | + | + | | | | | | | | | |
| Hex5HexNAc5dHex3 | 2304 | | | | | | + | + | | + | | | | | |
| Hex6HexNAc5dHex2 | 2320 | + | | | | | + | + | + | + | | | | | + |
| Hex7HexNAc5dHex | 2336 | | | | + | + | | | | | | | | | |
| Hex8HexNAc5 | 2352 | | | | + | + | | | | | | | | | |
| Hex7HexNAc6 | 2393 | | | | + | | + | + | + | + | | | | | + |
| Hex7HexNAc4dHex3 | 2425 | | | | + | + | | | | | | | | | |
| Hex6HexNAc5dHex3 | 2466 | | | | | | + | + | | + | | | | | |
| Hex8HexNAc5dHex | 2498 | | | | + | + | | | | | | | | | |
| Hex7HexNAc6dHex | 2539 | | | | + | | + | + | + | + | | | | | |
| Hex6HexNAc5dHex4 | 2612 | | | | | | + | + | | | | | | | |
| Hex8HexNAc7 | 2758 | | | | | | + | + | | | | | | | |
| HexNAc ≧ 3 and dHex ≧ 1 | | | | | | | | | | | | | | | |
| (including fucosylated N- | | | | | | | | | | | | | | | |
| glycans) | | | | | | | | | | | | | | | |
| Hex2HexNAc3dHex | 1120 | | + | | | | + | + | + | + | + | + | + | | + |
| Hex2HexNAc3dHex2 | 1266 | | | | | | | | + | | | | | | |
| Hex3HexNAc3dHex | 1282 | + | + | + | + | + | + | + | + | + | + | + | + | + | + |
| Hex3HexNAc3dHex2 | 1428 | | | | | | | + | | + | + | + | + | | + |
| Hex4HexNAc3dHex | 1444 | + | + | + | + | + | + | + | + | + | + | + | + | + | + |
| Hex4HexNAc3dHex2 | 1590 | + | | | | | + | + | + | + | + | + | + | | + |
| Hex5HexNAc3dHex | 1606 | + | + | + | + | + | + | + | + | + | + | + | + | + | + |
| Hex5HexNAc3dHex2 | 1752 | | | | | | + | + | + | + | | | | | |
| Hex6HexNAc3dHex | 1768 | + | + | | | + | + | + | + | + | | | + | | + |
| Hex3HexNAc4dHex | 1485 | + | + | + | + | + | + | + | + | + | + | + | + | + | + |
| Hex4HexNAc4dHex | 1647 | + | + | + | + | + | + | + | + | + | + | + | + | + | + |
| Hex3HexNAc5dHex | 1688 | + | + | + | + | + | + | + | + | + | + | + | + | + | + |
| Hex4HexNAc4dHex2 | 1793 | + | | | + | | | | + | + | + | + | + | | + |
| Hex5HexNAc4dHex | 1809 | + | + | + | + | + | + | + | + | + | + | + | + | + | + |
| Hex4HexNAc5dHex | 1850 | + | + | + | | | + | | + | + | + | | | | |
| Hex3HexNAc6dHex | 1891 | | | | | | | + | + | + | + | | | + | |
| Hex5HexNAc4dHex2 | 1955 | + | + | | + | | + | + | + | + | + | + | + | | + |
| Hex6HexNAc4dHex | 1971 | | + | | + | + | + | + | + | + | | | | | + |
| Hex4HexNAc5dHex2 | 1996 | + | + | | + | | + | + | + | + | | | | | |
| Hex5HexNAc5dHex | 2012 | + | + | + | + | | + | + | + | + | | | | | |
| Hex5HexNAc4dHex3 | 2101 | + | + | | + | | + | + | + | + | + | + | + | | + |
| Hex6HexNAc4dHex2 | 2117 | | | | | | + | + | | | | | | | |
| Hex7HexNAc4dHex | 2133 | | | | + | + | | + | | | | | | | + |
| Hex4HexNAc5dHex3 | 2142 | + | + | | + | + | + | + | | | | | | | + |
| Hex5HexNAc5dHex2 | 2158 | | + | | | | | + | + | + | | | | | |
| Hex6HexNAc5dHex | 2174 | + | + | + | + | | + | + | + | + | | + | | | + |
| Hex7HexNAc4dHex2 | 2279 | | | | + | + | | | | | | | | | |
| Hex5HexNAc5dHex3 | 2304 | | | | | | + | + | | + | | | | | |
| Hex6HexNAc5dHex2 | 2320 | + | | | | | + | + | + | + | | | | | + |
| Hex7HexNAc5dHex | 2336 | | | | + | + | | | | | | | | | |
| Hex7HexNAc4dHex3 | 2425 | | | | + | + | | | | | | | | | |
| Hex6HexNAc5dHex3 | 2466 | | | | | | + | + | | + | | | | | |
| Hex8HexNAc5dHex | 2498 | | | | + | + | | | | | | | | | |
| Hex7HexNAc6dHex | 2539 | | | | + | | + | + | + | + | | | | | |
| Hex6HexNAc5dHex4 | 2612 | | | | | | + | + | | | | | | | |
| HexNAc ≧ 3 and dHex ≧ 2 | | | | | | | | | | | | | | | |
| (including multifucosylated N- | | | | | | | | | | | | | | | |
| glycans) | | | | | | | | | | | | | | | |
| Hex2HexNAc3dHex2 | 1266 | | | | | | | | + | | | | | | |
| Hex3HexNAc3dHex2 | 1428 | | | | | | | + | | + | + | + | + | | + |
| Hex4HexNAc3dHex2 | 1590 | + | | | | | + | + | + | + | + | + | + | | + |
| Hex5HexNAc3dHex2 | 1752 | | | | | | + | + | + | + | | | | | |
| Hex4HexNAc4dHex2 | 1793 | + | | | + | | | | + | + | + | + | + | | + |
| Hex5HexNAc4dHex2 | 1955 | + | + | | + | | + | + | + | + | + | + | + | | + |
| Hex4HexNAc5dHex2 | 1996 | + | + | | + | | + | + | + | + | | | | | |
| Hex5HexNAc4dHex3 | 2101 | + | + | | + | | + | + | + | + | + | + | + | | + |
| Hex6HexNAc4dHex2 | 2117 | | | | | | + | + | | | | | | | |
| Hex4HexNAc5dHex3 | 2142 | + | + | | + | + | + | + | | | | | | | + |
| Hex5HexNAc5dHex2 | 2158 | | + | | | | | + | + | + | | | | | |
| Hex7HexNAc4dHex2 | 2279 | | | | + | + | | | | | | | | | |
| Hex5HexNAc5dHex3 | 2304 | | | | | | + | + | | + | | | | | |
| Hex6HexNAc5dHex2 | 2320 | + | | | | | + | + | + | + | | | | | + |
| Hex7HexNAc4dHex3 | 2425 | | | | + | + | | | | | | | | | |
| Hex6HexNAc5dHex3 | 2466 | | | | | | + | + | | + | | | | | |
| Hex6HexNAc5dHex4 | 2612 | | | | | | + | + | | | | | | | |
| HexNAc > Hex ≧ 2 | | | | | | | | | | | | | | | |
| (terminal HexNAc, N > H) | | | | | | | | | | | | | | | |
| Hex2HexNAc3 | 974 | | | | | | | + | + | + | | | | | |
| Hex2HexNAc3dHex | 1120 | | + | | | | + | + | + | + | + | + | + | | + |
| Hex2HexNAc3dHex2 | 1266 | | | | | | | | + | | | | | | |
| Hex3HexNAc4 | 1339 | | | | + | + | + | + | + | + | + | | | + | |
| Hex3HexNAc4dHex | 1485 | + | + | + | + | + | + | + | + | + | + | + | + | + | + |
| Hex3HexNAc5 | 1542 | + | + | + | + | + | | | | + | + | | | + | |
| Hex3HexNAc5dHex | 1688 | + | + | + | + | + | + | + | + | + | + | + | + | + | + |
| Hex4HexNAx5 | 1704 | + | + | + | + | + | | + | + | + | + | | + | + | + |
| Hex4HexNAc5dHex | 1850 | + | + | + | | | + | | + | + | + | | | | |
| Hex3HexNAc6dHex | 1891 | | | | | | | + | + | + | + | | | + | |
| Hex4HexNAc5dHex2 | 1996 | + | + | | + | | + | + | + | + | | | | | |
| Hex4HexNAc5dHex3 | 2142 | + | + | | + | + | + | + | | | | | | | + |
| HexNAc = Hex ≧ 5 | | | | | | | | | | | | | | | |
| (terminal HexNAc, N = H) | | | | | | | | | | | | | | | |
| Hex5HexNAc5 | 1866 | + | + | + | + | + | + | + | + | + | + | | | + | + |
| Hex5HexNAc5dHex | 2012 | + | + | + | + | | + | + | + | + | | | | | |
| Hex5HexNAc5dHex2 | 2158 | | + | | | | | + | + | + | | | | | |
| Hex6HexNAc6 | 2231 | | | | | | | + | | + | | | | | |
| Hex5HexNAc5dHex3 | 2304 | | | | | | + | + | | + | | | | | |

hESC, human embryonic stem cells;
EB, embryoid bodies derived from hESC;
st.3, stage 3 differentiated cells derived from hESC;
hEF, human fibroblast feeder cells;
mEF, murine fibroblast feeder cells;
BM MSC, bone-marrow derived mesenchymal stem cells;
OB, Osteoblast-differentiated cells derived from BM MSC;
CB MSC, cord blood derived mesenchymal stem cells;
OB, adipocyte-differentiated cells derived from CB MSC;
CB MNC, cord blood mononuclear cells;
CD34+, CD133+, LIN−, and CD8−: subpopulations of CB MNC.

TABLE 5

| | | | | | | | BM | | CB | | CB | | | | |
|---|---|---|---|---|---|---|---|---|---|---|---|---|---|---|---|
| Proposed composition | m/z | hESC | EB | st.3 | hEF | mEF | MSC | OB | MSC | AC | MNC | CD 34+ | CD 133+ | LIN− | CD 8− |
| HexNAc = 3 and Hex ≧ 2 | | | | | | | | | | | | | | | |
| (including hybrid-type and | | | | | | | | | | | | | | | |
| monoantennary N-glycans) | | | | | | | | | | | | | | | |
| Hex3HexNAc3dHexSP | 1338 | | | | | | | + | | | | | | | |
| Hex4HexNAc3SP | 1354 | | | | | | + | + | | | | | | | |
| NeuAcHex3HexNAc3 | 1403 | | + | | | + | + | + | | + | + | + | + | + | + |
| NeuGcHex3HexNAc3 | 1419 | | | | | | | | | | | | + | | |
| Hex4HexNAc3dHexSP | 1500 | + | + | | | + | + | + | + | | | + | + | + | + |
| Hex5HexNAc3SP | 1516 | | + | | | | + | + | | | | | | | + |
| NeuAcHex3HexNAc3dHex | 1549 | + | + | | + | + | + | + | | + | + | + | + | + | + |
| NeuAcHex3HexNAc3SP2 | 1563 | | | | | | | | | | | + | + | | |
| NeuAcHex4HexNAc3 | 1565 | + | + | | + | + | + | + | + | + | + | + | + | + | + |
| NeuGcHex4HexNAc3 | 1581 | | + | | + | | + | | | | | | + | + | |
| Hex4HexNAc3dHex2SP | 1646 | | | | | | | + | | | | + | | | |
| Hex5HexNAc3dHexSP | 1662 | | | | | | | + | | | | | | | |
| Hex6HexNAc3SP and/or | 1678 | + | + | | + | + | + | + | + | + | + | + | + | + | + |
| NeuAc2Hex2HexNAc3dHex | | | | | | | | | | | | | | | |
| NeuAc2Hex3HexNAc3 | 1694 | | | | | | + | | | | | | | | |
| NeuAcHex3HexNAc3dHexSP2 | 1709 | | | | | | | | | | | + | + | | |
| NeuAcHex4HexNAc3dHex | 1711 | + | + | + | + | + | + | + | + | + | + | + | + | + | + |
| NeuAcHex5HexNAc3 and/or | 1727 | + | + | | + | + | + | + | + | + | + | + | + | + | + |
| NeuGcHex4HexNAc3dHex | | | | | | | | | | | | | | | |
| NeuGcHex5HexNAc3 | 1743 | | | | | | + | | | | | | | | |
| NeuAcHex4HexNAc3dHexSP | 1791 | | + | | | | + | + | + | | | | + | + | |
| Hex5HexNAc3dHex2SP | 1808 | | | | | | | | | | | + | | | |
| NeuAc2Hex3HexNAc3dHex | 1840 | + | + | | | | + | | + | + | | | + | | + |
| NeuAc2Hex4HexNAc3 | 1856 | | | | | | + | | | | | + | | | |
| NeuAcHex4HexNAc3dHex2 | 1857 | | | | | | | | | | | + | + | | |
| NeuAcHex5HexNAc3dHex and/or | 1873 | + | + | + | + | + | + | + | + | + | + | + | + | + | + |
| NeuGcHex4HexNAc3dHex2 | | | | | | | | | | | | | | | |
| NeuAcHex6HexNAc3 | 1889 | + | + | | + | + | + | + | + | + | + | + | + | + | + |
| Hex8HexNAc3SP and/or | 2002 | + | + | | + | + | + | + | + | + | | + | | | + |
| NeuAc2Hex4HexNAc3dHex | | | | | | | | | | | | | | | |
| NeuAcHex4HexNAc3dHex3 | 2003 | | + | | | | + | | | | | | | | |
| NeuAc2Hex5HexNAc3 and/or | 2018 | | | | | | + | + | + | + | | + | + | + | |
| NeuGcNeuAcHex4HexNAc3dHex | | | | | | | | | | | | | | | |
| NeuAcHex5HexNAc3dHex2 | 2019 | | | | | | | | | | | + | + | + | |
| NeuGcNeuAcHex5HexNAc3 and/or | 2034 | | | | | | | | | | | + | | | |
| NeuGc2Hex4HexNAc3dHex | | | | | | | | | | | | | | | |
| NeuAcHex6HexNAc3dHex | 2035 | | + | | | | + | + | + | + | + | + | + | + | + |
| NeuGc2Hex5HexNAc3 | 2050 | | | | | | | | | | | | | + | |
| NeuAcHex7HexNAc3 | 2051 | | + | | | | + | + | | | | | + | + | + |
| NeuAc2Hex4HexNAc3dHexSP and/or | 2082 | | | | | | + | + | + | | | | | | |
| Hex8HexNAc3SP2 | | | | | | | | | | | | | | | |
| NeuAcHex6HexNAc3dHexSP | 2115 | | | | | | | | | | | | + | | |
| Hex8HexNAc3dHexSP and/or | 2148 | | | | | | | | | | | | + | | |
| NeuAc2Hex4HexNAc3dHex2 | | | | | | | | | | | | | | | |
| NeuAcHex8HexNAc3SP and/or | 2293 | | | | | | | | | | | | + | | |
| NeuAc3Hex4HexNAc3dHex | | | | | | | | | | | | | | | |
| NeuAc2Hex5HexNAc3dHex2 and/or | 2310 | | | | | | | | | + | | | | | |
| NeuGcNeuAcHex4HexNAc3dHex3 | | | | | | | | | | | | | | | |
| NeuAc3Hex5HexNAc3SP | 2389 | | | | | + | | | | | | | | | |
| NeuAc2Hex5HexNAc3dHex2SP | 2390 | + | + | + | + | + | + | | | | + | + | + | + | |
| NeuAc2Hex6HexNAc3dHexSP | 2406 | | | | | + | | | | | | + | + | | |
| NeuAcHex8HexNAc3dHexSP and/or | 2439 | | | | | | | | | | | + | | | |
| NeuAc3Hex4HexNAc3dHex2 | | | | | | | | | | | | | | | |
| NeuAcHex9HexNAc3dHex | 2521 | | | | | | | | | | | | + | | |
| HexNAc ≧ 4 and Hex ≧ 3 | | | | | | | | | | | | | | | |
| (including complex-type N- | | | | | | | | | | | | | | | |
| glycans) | | | | | | | | | | | | | | | |
| Hex4HexNAc4SP | 1557 | | + | | | + | | + | + | | | | | | |
| NeuAcHex3HexNAc4 | 1606 | | | | | | + | | | | | | | | |
| Hex4HexNAc4SP2 | 1637 | | + | | + | | + | + | | | | + | + | + | + |
| Hex4HexNAc4dHexSP | 1703 | | + | | | + | | + | | | | | | | |
| Hex4HexNAc4SP3 and/or | 1717 | | | | | | | | | | | | | + | |
| Hex7HexNAc2SP2 | | | | | | | | | | | | | | | |
| Hex5HexNAc4SP | 1719 | | + | | + | + | + | + | + | | | | | | |
| NeuAcHex3HexNAc4dHex | 1752 | | | | | | | | | | | | | | + |
| NeuAcHex4HexNAc4 | 1768 | | + | | + | + | + | + | + | + | + | + | + | + | + |
| NeuGcHex4HexNAc4 | 1784 | | | | | | | | | | | + | + | | |
| Hex5HexNAc4SP2 and/or | 1799 | | + | | | | + | | | | | | + | | |
| Hex8HexNAc2SP | | | | | | | | | | | | | | | |
| NeuAcHex3HexNAc5 | 1809 | | | | | | | + | | | | | | | |
| NeuGcHex3HexNAc5 | 1825 | | | | | | + | + | | | | | | | |
| Hex5HexNAc4dHexSP | 1865 | + | + | | + | + | + | + | + | | | + | + | + | + |
| Hex6HexNAcSP | 1881 | | | | | + | | | | | | | | | |
| Hex4HexNAc5dHexSP | 1906 | + | + | | | | | | | | | | | | |
| NeuAcHex4HexNAc4dHex | 1914 | + | + | | + | + | + | + | + | + | + | + | + | + | + |
| NeuAcHex4HexNAc4SP2 | 1928 | | | | | | | | | | | + | + | | |
| NeuAcHex5HexNAc4 | 1930 | + | + | + | + | + | + | + | + | + | + | + | + | + | + |
| NeuGcHex5HexNAc4 | 1946 | + | + | | + | | + | + | | | | + | + | + | |
| NeuAcHex4HexNAc5 | 1971 | + | + | | | + | + | | + | | | | + | | + |
| NeuAcHex5HexNAc4Ac | 1972 | | | | | | | | | + | | | | | |
| Hex5HexNAc5SP2 | 2002 | | | | + | + | + | + | + | + | | + | | | |
| NeuAcHex5HexNAc4SP | 2010 | | | | | | | + | | | | | + | | |
| Hex5HexNAc4dHex2SP | 2011 | | | | | | | | | | | | + | | |
| NeuGcHex5HexNAc4SP | 2026 | | | | | | | | | | | | + | | |
| Hex6HexNAc4dHexSP | 2027 | | | | | + | | | | | | | + | | |
| Hex7HexNAc4SP and/or | 2043 | | | | | | | + | | | | | | | |
| Hex4HexNAc6SP2 and/or | | | | | | | | | | | | | | | |
| NeuAc2Hex3HexNAc4dHex | | | | | | | | | | | | | | | |
| NeuAcHex4HexNAc5SP | 2051 | | + | | | | + | + | | | | | + | + | |
| Hex4HexNAc5dHex2SP | 2052 | + | + | | | | | + | + | | | | | | |
| NeuAc2Hex4HexNAc4 | 2059 | | | | | | + | | | | | | + | | |
| NeuAcHex4HexNAc4dHex2 | 2060 | | | | | | + | | + | + | | + | + | + | |
| NeuAcHex4HexNAc4dHexSP2 | 2074 | | | | | | | | | | | + | + | | |
| NeuAcHex5HexNAc4dHex | 2076 | + | + | + | + | + | + | + | + | + | + | + | + | + | + |
| NeuAcHex6HexNAc4 and/or | 2092 | + | + | | + | + | + | + | + | | + | + | + | + | + |
| NeuGcHex5HexNAc4dHex | | | | | | | | | | | | | | | |
| NeuAcHex3HexNAc5dHex2 and/or | 2101 | | | | | | | | | + | | | | | |
| NeuAc2Hex4HexNAc4Ac | | | | | | | | | | | | | | | |
| NeuGcHex6HexNAc4 | 2108 | | | | | | | | | | | | + | | |
| NeuAcHex4HexNAc5dHex | 2117 | + | + | + | | + | | | | | + | + | + | + | + |
| Hex4HexNAc5dHex2SP2 | 2132 | | | | + | | | | | | | | | | |
| NeuAcHex5HexNAc5 | 2133 | + | + | | | + | + | | + | | + | + | + | + | + |
| NeuAc2Hex4HexNAc4SP | 2139 | | | | | | | | | | | | | | |
| NeuAcHex5HexNAc4dHexSP | 2156 | + | + | | | | | + | + | | | | + | + | + |
| Hex5HexNAc4dHex3SP | 2157 | | | | | | | | | | | | + | | |
| Hex6HexNAc5SP2 | 2164 | + | + | | | | + | | | | | | | | |
| Hex6HexNAc4dHex2SP and/or | 2173 | | | | | | | | | | | | | + | |
| Hex3HexNAc6dHex2SP2 | | | | | | | | | | | | | | | |
| NeuAcHex4HexNAc6 | 2174 | + | | | | | | + | + | + | | | + | + | |
| NeuAc3Hex3HexNAc4 and/or | 2188 | | | | | | | + | | | | | + | | |
| NeuGcHex6HexNAc4SP and/or | | | | | | | | | | | | | | | |
| NeuAc2NeuGcHex2HexNAc4dHex | | | | | | | | | | | | | | | |
| NeuAc2Hex3HexNAc4dHex2 and/or | 2189 | | | | | + | + | | | | | | | | |
| Hex7HexNAc4dHexSP and/or | | | | | | | | | | | | | | | |
| Hex4HexNAc6dHexSP2 | | | | | | | | | | | | | | | |
| NeuAc2Hex4HexNAc4dHex | 2205 | | | | | | | + | | | | | | | |
| NeuAc2Hex4HexNAc4SP2 | 2219 | | | | | | | | | | | + | | | |
| NeuAc2Hex5HexNAc4 | 2221 | + | + | + | + | + | + | + | + | + | + | + | + | + | + |
| NeuAcHex5HexNAc4dHex2 | 2222 | + | + | + | + | + | + | + | + | + | + | + | + | + | + |
| Hex6HexNAc5dHexSP | 2230 | | + | | + | | | + | + | | | | | | |
| NeuGcNeuAcHex5HexNAc4 | 2237 | + | + | | + | | | | | + | | + | + | + | |
| NeuAcHex6HexNAc4dHex and/or | 2238 | + | + | + | + | + | + | + | + | + | + | + | + | + | + |
| NeuGcHex5HexNAc4dHex2 | | | | | | | | | | | | | | | |
| NeuAc2Hex3HexNAc5dHex and/or | 2246 | | | | | | + | + | + | + | | | | | |
| Hex7HexNAc5SP | | | | | | | | | | | | | | | |
| NeuGc2Hex5HexNAc4 | 2253 | + | + | | + | | | | | | | + | + | + | |
| NeuAcHex7HexNAc4 and/or | 2254 | + | + | | + | + | + | + | + | | | + | + | + | |
| NeuGcHex6HexNAc4dHex | | | | | | | | | | | | | | | |
| NeuAc2Hex4HexNAc5 | 2262 | | | | | | | + | | | | | | | |
| NeuAcHex4HexNAc5dHex2 and/or | 2263 | + | | | | | | | | + | | | + | | |
| NeuAc2Hex5HexNAc4Ac | | | | | | | | | | | | | | | |
| NeuAcHex5HexNAc5dHex | 2279 | + | + | + | + | + | + | + | + | + | + | + | + | + | + |
| NeuAc2Hex4HexNAc4dHexSP and/or | 2285 | | | | | | | | | | | | | + | |
| Hex11HexNAc2SP | | | | | | | | | | | | | | | |
| NeuAcHex6HexNAc5 | 2295 | + | + | | + | + | + | + | + | + | + | + | + | + | + |
| NeuAc2Hex5HexNAc4SP | 2301 | | | | | | | | | | | | + | | |
| NeuAcHex5HexNAc4dHex2SP | 2302 | | | | | | | | | | | | + | | |
| NeuAc2Hex5HexNAc4Ac2 | 2305 | | | | | | | | | + | | | | | |
| Hex6HexNAc4dHex3SP and/or | 2319 | | | | | | | | + | | | | + | + | |
| NeuGcNeuAcHex3HexNAc6 | | | | | | | | | | | | | | | |
| NeuAcHex4HexNAc6dHex | 2320 | | | | | | | | | | | | + | + | |
| NeuAcHex5HexNAc5dHexAc | 2321 | | | | | | | | + | + | | | | | |
| Hex7HexNAc4dHex2SP and/or | 2335 | | | | | | | | | | | | + | + | |
| Hex4HexNAc6dHex2SP2 | | | | | | | | | | | | | | | |
| NeuAcHex5HexNAc6 | 2338 | | | | | | | | | | | + | | + | |
| NeuAc3Hex4HexNac4 | 2350 | | | | | + | | | | | | | | | |
| NeuAc2Hex4HexNAc4dHexSP | 2365 | | | | | | | | | | | + | + | + | |
| NeuAcHex5HexNAc4dHex | 2367 | + | + | + | + | + | + | + | + | + | + | + | + | + | + |
| NeuAcHex5HexNAc4dHex3 | 2368 | + | + | | + | + | + | + | + | + | + | + | + | + | + |
| NeuAc2Hex6HexNAc4 and/or | 2383 | + | + | | + | + | + | + | | | | + | + | + | |
| NeuGcNeuAcHex5HexNAc4dHex | | | | | | | | | | | | | | | |
| NeuAcHex6HexNAc4dHex2 and/or | 2384 | + | + | | + | + | | | + | | | + | + | | |
| NeuGcHex5HexNAc4dHex3 | | | | | | | | | | | | | | | |
| NeuAc2Hex3HexNAc5dHex2 and/or | 2392 | | | | | + | + | | | | | | | | |
| Hex7HexNAc5dHexSP | | | | | | | | | | | | | | | |
| NeuAcHex3HexNAc5dHex4 | 2393 | | | | | + | | | | | | | | | |
| NeuGc2Hex5HexNAc4dHex | 2399 | | + | | | | | | | | | | + | + | |
| NeuAcHex4HexNAc6dHexSP and/or | 2400 | | | | | | | | | | | | + | | |
| NeuGcHex6HexNAc4dHex2 and/or | | | | | | | | | | | | | | | |
| NeuAcHex7HexNAc4dHex | | | | | | | | | | | | | | | |
| NeuAc2Hex4HexNAc5dHex | 2408 | + | + | | | | | | | | | | | + | |
| NeuAcHex4HexNAc5dHex3 and/or | 2409 | | | | | | | + | | | | | + | | |
| NeuAc2Hex5HexNAc4dHexAc | | | | | | | | | | | | | | | |
| NeuAc2Hex5HexNAc5 | 2424 | | | | | | + | | | | + | + | | + | + |
| NeuAcHex5HexNAc5dHex2 | 2425 | + | + | | | | | + | + | + | + | + | + | + | + |
| NeuAcHex6HexNAc5dHex | 2441 | + | + | + | + | + | + | + | + | + | + | + | + | + | + |
| NeuAc2Hex5HexNAc4dHexSP | 2447 | | + | | | | | + | + | | | + | + | + | + |
| NeuAcHex5HexNAc4dHex3SP | 2448 | | + | | | | | | | | | + | + | + | + |
| NeuAcHex7HexNAc5 and/or | 2457 | + | | | + | + | + | + | | | | | | | |
| NeuGcHex6HexNAc5dHex | | | | | | | | | | | | | | | |
| NeuGcHex7HexNAc5 | 2473 | | | | | | | | | | | + | + | | |
| NeuAcHex5HexNAc6dHex | 2482 | | | | | | | | | | | | | + | |
| NeuAcHex4HexNAc5dHex3SP | 2489 | | | | | | | | | | | + | + | | |
| Hex6HexNAc7SP | 2490 | | | | | | | | | | | | + | | |
| NeuAc3Hex5HexNAc4 | 2512 | | + | | | | | | | | | + | + | + | |
| NeuAc2Hex5HexNAc4dHex2 | 2513 | | | | | | + | + | + | | | + | + | + | + |
| NeuAcHex5HexNAc4dHex4 | 2514 | | | | | | + | | | | | | | | + |
| NeuAcHex6HexNAc5dHexSP and/or | 2521 | + | + | | | | | + | | | | | + | | |
| NeuAc3Hex2HexNAc5dHex2 | | | | | | | | | | | | | | | |
| Hex6HexNAc5dHex3SP | 2522 | | | | | | | + | | | | | + | | |
| NeuGcNeuAc2Hex5HexNAc4 | 2528 | + | + | | | | | | | | | + | + | + | |
| NeuAc2Hex6HexNAc4dHex and/or | 2529 | | + | | | | | | | | | + | + | + | |
| NeuGcNeuAcHex5HexNAc4dHex2 | | | | | | | | | | | | | | | |
| NeuGc2NeuAcHex5HexNAc4 | 2544 | + | + | | | | | | | + | | + | + | + | |
| NeuGc2Hex5HexNAc4dHex2 and/or | 2545 | | | | | | | | | | | + | + | + | |
| NeuGcNeuAcHex6HexNAc4dHex | | | | | | | | | | | | | | | |
| NeuGc3Hex5HexNAc4 | 2560 | | + | | | | | | | | | + | + | + | |
| NeuGc2Hex6HexNAc4dHex | 2561 | | | | | | | | | | | | + | | |
| NeuAc2Hex5HexNAc5dHex | 2570 | + | + | | | | | + | | | + | + | + | + | + |
| NeuAcHex5HexNAc5dHex3 | 2571 | | + | + | | | | + | | | + | + | + | + | + |
| NeuAc2Hex6HexNAc5 | 2588 | | + | | + | | + | + | + | + | + | + | + | + | + |
| NeuAcHex6HexNAc5dHex2 | 2587 | + | + | | + | | + | + | + | + | + | + | + | + | + |
| Hex7HexNAc6dHexSP | 2595 | | | | | | | + | | | | | | | |
| NeuGcNeuAcHex6HexNAc5 | 2602 | | | | | | | | | | | + | + | + | |
| NeuAcHex7HexNAc5dHex and/or | 2603 | | + | | | + | + | + | + | | | + | + | | |
| NeuGcHex6HexNAc5dHex2 | | | | | | | | | | | | | | | |
| NeuAcHex8HexNAc5 and/or | 2619 | | | | | + | | | | | | + | + | | |
| NeuGcHex7HexNAc5dHex | | | | | | | | | | | | | | | |
| NeuAc2Hex5HexNAc6 | 2627 | | | | | | | | + | | | | | | |
| NeuGcHex8HexNAc5 and/or | 2635 | | | | | | | | | | | + | + | | |
| NeuAcHex4HexNAc5dHex4SP | | | | | | | | | | | | | | | |
| NeuAcHex6HexNAc6dHex | 2644 | | + | + | | | + | + | + | | + | + | + | + | + |
| NeuAc2Hex5HexNAc4dHex3 | 2659 | | | | | | | + | | | | + | | | |
| NeuAcHex7HexNAc6 | 2660 | + | | | + | | + | + | + | + | + | + | + | | + |
| NeuGcNeuAc2Hex5HexNAc4dHex | 2674 | | | | | | + | + | | | | | | | |
| and/or NeuAc3Hex6HexNAc4 | | | | | | | | | | | | | | | |
| NeuAc2Hex4HexNAc5dHex2SP2 | 2714 | | | | | | | + | | | | + | + | + | |
| NeuAcHex4HexNAc5dHex4SP2 and/or | 2715 | | | | | | | | | | | | + | + | |
| NeuAc3Hex5HexNAc5 | | | | | | | | | | | | | | | |
| NeuAc2Hex5HexNAc5dHex2 | 2716 | | | | | | | | | | | | | + | |
| NeuAc2Hex6HexNAc5dHex | 2732 | + | + | + | + | | + | + | + | + | + | + | + | + | + |
| NeuAcHex6HexNAc5dHex3 | 2733 | + | + | | + | + | + | + | + | + | + | + | + | + | + |
| NeuGcNeuAcHex6HexNAc5dHex | 2748 | | | | | | | | | | | | + | | |
| NeuAcHex8HexNAc5dHex | 2765 | | | | | + | | | | | | | | | |
| NeuGcHex8HexNAc5dHex and/or | 2781 | | | | | | | | | | | | + | | |
| NeuAcHex9HexNAc5 | | | | | | | | | | | | | | | |
| NeuAcHex6HexNAc6dHex2 | 2791 | | | | | | | | | | | + | + | + | + |
| Hex6HexNAc6dHex3SP2 | 2805 | | | | | | | | | | | | | + | |
| NeuAcHex7HexNAc6dHex | 2807 | + | + | + | + | | + | + | + | + | + | + | + | + | + |
| NeuAc2Hex6HexNAc5dHexSP | 2812 | | | | | | | | + | | | + | + | + | + |
| NeuAcHex6HexNAc5dHex3SP | 2813 | | | | | | | | | | | | + | | |
| NeuGcNeuAc3Hex5HexNAc4 | 2819 | | | | | | | | | | | | + | | |
| NeuAc3Hex6HexNAc4dHex and/or | 2820 | | | | | | | | | | | | + | | |
| NeuGcNeuAc2Hex5HexNAc4dHex2 | | | | | | | | | | | | | | | |
| NeuAc3Hex6HexNAc5 | 2878 | + | + | | + | + | + | + | + | + | + | + | + | + | |
| NeuAc2Hex6HexNAc5dHex2 | 2879 | + | + | | + | + | + | + | + | + | + | + | + | + | + |
| NeuAcHex6HexNAc5dHex4 | 2880 | | | | | | + | | | | | + | + | + | + |
| NeuGcNeuAc2Hex6HexNAc5 | 2894 | | | | | | | + | | | | | + | | |
| NeuAc2Hex7HexNAc5dHex and/or | 2895 | | | | | + | | | | | | | + | | |
| NeuGcNeuAcHex6HexNAc5dHex2 | | | | | | | | | | | | | | | |
| NeuAc3Hex6HexNAc4dHexSP and/or | 2900 | | | | | | + | | | | | | | | |
| NeuGcNeuAc2Hex5HexNAc4dHex2SP | | | | | | | | | | | | | | | |
| NeuGc2Hex6HexNAc5dHex2 | 2911 | | | | | | | | | | | | | + | |
| NeuAc2Hex5HexNAc6dHex2 | 2920 | | | | | | | | | + | | | | | |
| NeuGc3Hex6HexNAc5 | 2925 | | | | | | | | | | | | + | | |
| NeuGcNeuAc2Hex5HexNAc6 | 2935 | | | | | | | | | | | + | | | |
| NeuAc2Hex6HexNAc6dHex and/or | 2936 | | + | + | | | | | | + | | + | + | + | + |
| NeuGcNeuAcHex5HexNAc6dHex2 | | | | | | | | | | | | | | | |
| NeuAcHex6HexNAc6dHex3 | 2937 | | | | | | | + | | | | | | + | |
| NeuGc2NeuAcHex5HexNAc6 and/or | 2951 | | | | | | | | + | | | | | | |
| NeuAc3Hex5HexNAc4dHex3 | | | | | | | | | | | | | | | |
| NeuAc2Hex7HexNAc6 | 2952 | | | | | | + | + | + | + | | | | + | + |
| NeuAcHex7HexNAc6dHex2 | 2953 | + | | | + | | + | + | + | | | + | + | | + |
| Hex8HexNAc7dHexSP | 2961 | | | | | | | | | + | | | | | |
| NeuAc2Hex4HexNAc7dHex2 | 2961 | | | | | | | | | + | | | | | |
| NeuAcHex7HexNAc7dHex | 3010 | | + | | | | | | | | | | + | + | |
| NeuAc3Hex6HexNAc5dHex | 3024 | + | + | | + | | + | + | + | + | + | + | + | + | + |
| NeuAc2Hex6HexNAc5dHex3 | 3025 | + | + | | + | | + | + | | + | + | + | + | + | + |
| NeuAcHex8HexNAc7 | 3026 | | | + | + | | + | | | | | + | + | | + |
| NeuGc3Hex6HexNAc5dHex and/or | 3072 | | | | | | | | | | | | + | | |
| NeuGc2NeuAcHex7HexNAc5 | | | | | | | | | | | | | | | |
| NeuAc2Hex6HexNAc6dHex2 | 3082 | | | | | | | | | | | | | + | |
| NeuAc2Hex7HexNAc6dHex | 3098 | + | + | + | + | | + | + | + | + | + | + | + | + | + |
| NeuAcHex7HexNAc6dHex3 | 3099 | + | + | | + | | + | + | + | + | + | + | + | + | + |
| NeuAc3Hex6HexNAc5dHexSP | 3104 | | | | | | | | | | | + | | + | |
| NeuAc2Hex6HexNAc5dHex3SP | 3105 | | | | | | | | | | | + | + | | |
| NeuAc3Hex6HexNAc5dHex2 | 3170 | | | | | | | | | | | + | + | | |
| NeuAc2Hex6HexNAc5dHex4 | 3171 | | + | | + | | + | + | | | | | + | + | |
| NeuAcHex8HexNAc7dHex | 3172 | + | | | + | | + | + | + | + | + | + | + | + | + |
| NeuAc3Hex6HexNAc6dHex | 3227 | | | | | | | | | | | + | | + | |
| NeuAc2Hex6HexNAc6dHex3 | 3228 | | | | | | | | | | | | | + | |
| NeuAc3Hex7HexNAc6 | 3243 | | | | | | | + | + | | | | | + | |
| NeuAc2Hex7HexNAc6dHex2 | 3244 | | | | | | | | + | + | | + | + | + | |
| NeuAcHex7HexNAc6dHex4 | 3245 | + | | | | | + | | + | | | + | + | + | |
| NeuAc2Hex7HexNAc7dHex | 3301 | | | | | | | | | | | | | + | |
| NeuAcHex7HexNAc7dHex3 | 3302 | | | | | | | | | | | | + | | |
| NeuAc2Hex8HexNAc7 | 3317 | + | | | | | | + | | | | + | + | | |
| NeuAcHex8HexNAc7dHex2 | 3318 | | | | | | | | + | | | + | + | | |
| NeuAc3Hex7HexNAc6dHex | 3389 | | | | | | + | + | + | + | | + | + | + | |
| NeuAc2Hex7HexNAc6dHex3 | 3390 | + | + | | | | + | + | + | + | | + | + | + | + |
| NeuAcHex7HexNAc6dHex5 and/or | 3391 | | | | + | | + | | | | | | + | | |
| NeuAcHex9HexNAc8 | | | | | | | | | | | | | | | |
| NeuAc2Hex8HexNAc7dHex | 3463 | + | | | + | | + | + | + | + | | + | + | | + |
| NeuAcHex8HexNAc7dHex3 | 3464 | | | | | | + | | + | + | | + | + | | + |
| NeuAc2Hex7HexNAc6dHex4 | 3536 | | | | | | + | + | | | | + | + | + | + |
| NeuAcHex9HexNAc8dHex | 3537 | | | | + | | + | + | + | | | | + | | |
| NeuAc3Hex8HexNAc7 | 3608 | + | | | | | | | | | | | | + | |
| NeuAc2Hex8HexNac7dHex2 | 3609 | | | | | | | + | + | | | + | | | |
| NeuAcHex8HexNac7dHex4 | 3610 | + | | | | | | | + | | | + | + | | |
| NeuAc4Hex7HexNAc6dHex | 3680 | | | | | | | | + | | | + | | + | |
| NeuAc3Hex7HexNAc6dHex3 | 3681 | | | | + | | + | | + | + | | + | + | + | |
| NeuAc2Hex9HexNAc8 | 3682 | + | | | | | | | + | + | | | | | |
| NeuAcHex9HexNAc8dHex2 | 3683 | | | | | | | + | | | | + | + | | |
| NeuAc3Hex8HexNAc7dHex | 3754 | | | | | | + | + | + | | | | | | + |
| NeuAc2Hex8HexNAc7dHex3 | 3755 | | | | + | | | | + | + | | + | + | + | |
| NeuAcHex10HexNAc9 and/or | 3756 | + | | | | | | | + | + | | | + | | |
| NeuAcHex8HexNAc7dHex5 | | | | | | | | | | | | | | | |
| NeuAc4Hex6HexNAc8 | 3778 | | | | | | | | | | | + | | | |
| NeuAc3Hex7HexNAc6dHex4 | 3827 | | | | | | + | | | | | | | | + |
| NeuAc2Hex9HexNAc8dHex | 3828 | | | | + | | | + | + | | | + | | | |
| NeuAcHex9HexNAc8dHex3 | 3829 | | | | | | | + | + | | | + | + | | |
| NeuAc2Hex8HexNAc7dHex4 | 3901 | | | | | | | + | | | | + | | + | |
| NeuAc2Hex9HexNAc8dHex2 | 3974 | | | | | | | | | | | + | | + | |
| NeuAcHex9HexNAc8dHex4 | 3975 | | | | | | | | | | | + | + | | |
| NeuAc4Hex8HexNAc7dHex | 4045 | | | | | | | | | | | | | + | |
| NeuAc3Hex8HexNAc7dHex3 | 4046 | | | | | | | | | | | | + | + | |
| NeuAc2Hex10HexNAc9 and/or | 4047 | | | | + | | | | + | | | | | | |
| NeuAc2Hex8HexNAc7dHex5 | | | | | | | | | | | | | | | |
| NeuAc3Hex9HexNAc8dHex | 4119 | | | | | | | | | | | | | + | |
| NeuAc2Hex9HexNAc8dHex3 | 4120 | | | | | | | | | | | | + | | |
| HexNAc ≧ 3 and dHex ≧ 1 | | | | | | | | | | | | | | | |
| (including fucosylated N- | | | | | | | | | | | | | | | |
| glycans) | | | | | | | | | | | | | | | |
| Hex3HexNAc3dHexSP | 1338 | | | | | | | + | | | | | | | |
| Hex4HexNAc3dHexSP | 1500 | + | + | | | + | + | + | + | | | + | + | + | + |
| NeuAcHex3HexNAc3dHex | 1549 | + | + | | + | + | + | + | | + | + | + | + | + | + |
| Hex4HexNAc3dHex2SP | 1646 | | | | | | | + | | | | | + | | |
| Hex5HexNAc3dHexSP | 1662 | | | | | | | + | | | | | | | |
| Hex6HexNAc3SP and/or | 1678 | + | + | | + | + | + | + | + | + | + | + | + | + | + |
| NeuAc2Hex2HexNAc3dHex | | | | | | | | | | | | | | | |
| NeuAcHex3HexNAc3dHexSP2 | 1709 | | | | | | | | | | | + | + | | |
| NeuAcHex4HexNAc3dHex | 1711 | + | + | + | + | + | + | + | + | + | + | + | + | + | + |
| NeuAcHex5HexNAc3 and/or | 1727 | + | + | | + | + | + | + | + | + | + | + | + | + | + |
| NeuGcHex4HexNAc3dHex | | | | | | | | | | | | | | | |
| NeuAcHex4HexNAc3dHexSP | 1791 | | + | | | | + | + | + | | | | + | + | |
| Hex5HexNAc3dHex2SP | 1808 | | | | | | | | | | | + | | | |
| NeuAc2Hex3HexNAc3dHex | 1840 | + | + | | | | + | | + | + | | | + | | + |
| NeuAcHex4HexNAc3dHex2 | 1857 | | | | | | | | | | | + | + | | |
| NeuAcHex5HexNAc3dHex and/or | 1873 | + | + | + | + | + | + | + | + | + | + | + | + | + | + |
| NeuGcHex4HexNAc3dHex2 | | | | | | | | | | | | | | | |
| Hex8HexNAc3SP and/or | 2002 | + | + | | + | + | + | + | + | + | | + | | | + |
| NeuAc2Hex4HexNAc3dHex | | | | | | | | | | | | | | | |
| NeuAcHex4HexNAc3dHex3 | 2003 | | + | | | | + | | | | | | | | |
| NeuAc2Hex5HexNAc3 and/or | 2018 | | | | | | + | + | + | + | | + | + | + | |
| NeuGcNeuAcHex4HexNAc3dHex | | | | | | | | | | | | | | | |
| NeuAcHex5HexNAc3dHex2 | 2019 | | | | | | | | | | | + | + | + | |
| NeuGcNeuAcHex5HexNAc3 and/or | 2034 | | | | | | | | | | | + | | | |
| NeuGc2Hex4HexNAc3dHex | | | | | | | | | | | | | | | |
| NeuAcHex6HexNAc3dHex | 2035 | | + | | | | + | + | + | + | + | + | + | + | + |
| NeuAc2Hex4HexNAc3dHexSP and/or | 2082 | | | | | | + | + | + | | | | | | |
| Hex8HexNAc3SP2 | | | | | | | | | | | | | | | |
| NeuAcHex6HexNAc3dHexSP | 2115 | | | | | | | | | | | | + | | |
| Hex8HexNAc3dHexSP and/or | 2148 | | | | | | | | | | | | + | | |
| NeuAc2Hex4HexNAc3dHex2 | | | | | | | | | | | | | | | |
| NeuAcHex8HexNAc3SP and/or | 2293 | | | | | | | | | | | | + | | |
| NeuAc3Hex4HexNAc3dHex | | | | | | | | | | | | | | | |
| NeuAc2Hex5HexNAc3dHex2 and/or | 2310 | | | | | | | | | + | | | | | |
| NeuGcNeuAcHex4HexNAc3dHex3 | | | | | | | | | | | | | | | |
| NeuAc2Hex5HexNAc3dHex2SP | 2390 | + | + | + | + | + | + | | | | + | + | + | + | |
| NeuAc2Hex6HexNAc3dHexSP | 2406 | | | | | + | | | | | | + | + | | |
| NeuAcHex8HexNAc3dHexSP and/or | 2439 | | | | | | | | | | | + | | | |
| NeuAc3Hex4HexNAc3dHex2 | | | | | | | | | | | | | | | |
| NeuAcHex9HexNAc3dHex | 2521 | | | | | | | | | | | | + | | |
| Hex4HexNAc4dHexSP | 1703 | | + | | | + | | + | | | | | | | |
| NeuAcHex3HexNAc4dHex | 1752 | | | | | | | | | | | | | | + |
| Hex5HexNAc4dHexSP | 1865 | + | + | | + | + | + | + | + | | | + | + | + | + |
| Hex4HexNAc5dHexSP | 1906 | + | + | | | | | | | | | | | | |
| NeuAcHex4HexNAc4dHex | 1914 | + | + | | + | + | + | + | + | + | + | + | + | + | + |
| Hex5HexNAc4dHex2SP | 2011 | | | | | | | | | | | | + | | |
| Hex6HexNAc4dHexSP | 2027 | | | | | + | | | | | | | + | | |
| Hex7HexNAc4SP and/or | 2043 | | | | | | | + | | | | | | | |
| Hex4HexNAc6SP2 and/or | | | | | | | | | | | | | | | |
| NeuAc2Hex3HexNAc4dHex | | | | | | | | | | | | | | | |
| Hex4HexNAc5dHex2SP | 2052 | + | + | | | | | + | + | | | | | | |
| NeuAcHex4HexNAc4dHex2 | 2060 | | | | | | + | | + | + | | + | + | + | |
| NeuAcHex4HexNAc4dHexSP2 | 2074 | | | | | | | | | | | + | + | | |
| NeuAcHex5HexNAc4dHex | 2076 | + | + | + | + | + | + | + | + | + | + | + | + | + | + |
| NeuAcHex6HexNAc4 and/or | 2092 | + | + | | + | + | + | + | + | | + | + | + | + | + |
| NeuGcHex5HexNAc4dHex | | | | | | | | | | | | | | | |
| NeuAcHex3HexNAc5dHex2 and/or | 2101 | | | | | | | | | + | | | | | |
| NeuAc2Hex4HexNAc4Ac | | | | | | | | | | | | | | | |
| NeuAcHex4HexNAc5dHex | 2117 | + | + | + | | + | | | | | + | + | + | + | + |
| Hex4HexNAc5dHex2SP2 | 2132 | | | | + | | | | | | | | | | |
| NeuAcHex5HexNAc4dHexSP | 2156 | + | + | | | | | + | + | | | | + | + | + |
| Hex5HexNAc4dHex3SP | 2157 | | | | | | | | | | | | + | | |
| Hex6HexNAc4dHex2SP and/or | 2173 | | | | | | | | | | | | | + | |
| Hex3HexNAc6dHex2SP2 | | | | | | | | | | | | | | | |
| NeuAc3Hex3HexNAc4 and/or | 2188 | | | | | | | + | | | | | + | | |
| NeuGcHex6HexNAc4SP and/or | | | | | | | | | | | | | | | |
| NeuAc2NeuGcHex2HexNAc4dHex | | | | | | | | | | | | | | | |
| NeuAc2Hex3HexNAc4dHex2 and/or | 2189 | | | | | + | + | | | | | | | | |
| Hex7HexNAc4dHexSP and/or | | | | | | | | | | | | | | | |
| Hex4HexNAc6dHexSP2 | | | | | | | | | | | | | | | |
| NeuAc2Hex4HexNAc4dHex | 2205 | | | | | | | + | | | | | | | |
| NeuAcHex5HexNAc4dHex2 | 2222 | + | + | + | + | + | + | + | + | + | + | + | + | + | + |
| Hex6HexNAc5dHexSP | 2230 | | + | | + | | | + | + | | | | | | |
| NeuAcHex6HexNAc4dHex and/or | 2238 | + | + | | + | + | + | + | + | + | + | + | + | + | + |
| NeuGcHex5HexNAc4dHex2 | | | | | | | | | | | | | | | |
| NeuAc2Hex3HexNAc5dHex and/or | 2246 | | | | | | + | + | + | + | | | | | |
| Hex7HexNAc5SP | | | | | | | | | | | | | | | |
| NeuAcHex7HexNAc4 and/or | 2254 | + | + | | + | + | + | + | + | | | + | + | + | |
| NeuGcHex6HexNAc4dHex | | | | | | | | | | | | | | | |
| NeuAcHex4HexNAc5dHex2 and/or | 2263 | + | | | | | | | | + | | | + | | |
| NeuAc2Hex5HexNAc4Ac | | | | | | | | | | | | | | | |
| NeuAcHex5HexNAc5dHex | 2279 | + | + | + | + | + | + | + | + | + | + | + | + | + | + |
| NeuAc2Hex4HexNAc4dHexSP and/or | 2285 | | | | | | | | | | | | | + | |
| Hex11HexNAc2SP | | | | | | | | | | | | | | | |
| NeuAcHex5HexNAc4dHex2SP | 2302 | | | | | | | | | | | | + | | |
| Hex6HexNAc4dHex3SP and/or | 2319 | | | | | | | | + | | | | + | + | |
| NeuGcNeuAcHex3HexNAc6 | | | | | | | | | | | | | | | |
| NeuAcHex4HexNAc6dHex | 2320 | | | | | | | | | | | | + | + | |
| NeuAcHex5HexNAc5dHexAc | 2321 | | | | | | | | + | + | | | | | |
| Hex7HexNAc4dHex2SP and/or | 2335 | | | | | | | | | | | | + | + | |
| Hex4HexNAc6dHex2SP2 | | | | | | | | | | | | | | | |
| NeuAc2Hex4HexNAc4dHexSP | 2365 | | | | | | | | | | | + | + | + | |
| NeuAc2Hex5HexNAc4dHex | 2367 | + | + | + | + | + | + | + | + | + | + | + | + | + | + |
| NeuAcHex5HexNAc4dHex3 | 2368 | + | + | | + | + | + | + | + | + | + | + | + | + | + |
| NeuAc2Hex6HexNAc4 and/or | 2383 | + | + | | + | + | + | + | | | | + | + | + | |
| NeuGcNeuAcHex5HexNAc4dHex | | | | | | | | | | | | | | | |
| NeuAcHex6HexNAc4dHex2 and/or | 2384 | + | + | | + | + | | | + | | | + | + | | |
| NeuGcHex5HexNAc4dHex3 | | | | | | | | | | | | | | | |
| NeuAc2Hex3HexNAc5dHex2 and/or | 2392 | | | | | + | + | | | | | | | | |
| Hex7HexNAc5dHexSP | | | | | | | | | | | | | | | |
| NeuAcHex3HexNAc5dHex4 | 2393 | | | | | + | | | | | | | | | |
| NeuGc2Hex5HexNAc4dHex | 2399 | | + | | | | | | | | | | + | + | |
| NeuAcHex4HexNAc6dHexSP and/or | 2400 | | | | | | | | | | | | + | | |
| NeuGcHex6HexNAc4dHex2 and/or | | | | | | | | | | | | | | | |
| NeuAcHex7HexNAc4dHex | | | | | | | | | | | | | | | |
| NeuAc2Hex4HexNAc5dHex | 2408 | + | + | | | | | | | | | | | + | |
| NeuAcHex4HexNAc5dHex3 and/or | 2409 | | | | | | | + | | | | | + | | |
| NeuAc2Hex5HexNAc4dHexAc | | | | | | | | | | | | | | | |
| NeuAcHex5HexNAc5dHex2 | 2425 | + | + | | | | | + | + | + | + | + | + | + | + |
| NeuAcHex6HexNAc5dHex | 2441 | + | + | + | + | + | + | + | + | + | + | + | + | + | + |
| NeuAc2Hex5HexNAc4dHexSP | 2447 | | + | | | | | + | + | | | + | + | + | + |
| NeuAcHex5HexNAc4dHex3SP | 2448 | | + | | | | | | | | | + | + | + | + |
| NeuAcHex7HexNAc5 and/or | 2457 | + | | | + | + | + | + | | | | | | | |
| NeuGcHex6HexNAc5dHex | | | | | | | | | | | | | | | |
| NeuAcHex5HexNAc6dHex | 2482 | | | | | | | | | | | | | + | |
| NeuAcHex4HexNAc5dHex3SP | 2489 | | | | | | | | | | | + | + | | |
| NeuAc2Hex5HexNAc4dHex2 | 2513 | | | | | | + | + | + | | | + | + | + | + |
| NeuAcHex5HexNAc4dHex4 | 2514 | | | | | | + | | | | | | | | + |
| NeuAcHex6HexNAc5dHexSP and/or | 2521 | + | + | | | | | + | | | | | + | | |
| NeuAc3Hex2HexNAc5dHex2 | | | | | | | | | | | | | | | |
| Hex6HexNAc5dHex3SP | 2522 | | | | | | | + | | | | | + | | |
| NeuAc2Hex6HexNAc4dHex and/or | 2529 | | + | | | | | | | | | + | + | + | |
| NeuGcNeuAcHex5HexNAc4dHex2 | | | | | | | | | | | | | | | |
| NeuGc2Hex5HexNAc4dHex2 and/or | 2545 | | | | | | | | | | | + | + | + | |
| NeuGcNeuAcHex6HexNAc4dHex | | | | | | | | | | | | | | | |
| NeuGc2Hex6HexNAc4dHex | 2561 | | | | | | | | | | | | + | | |
| NeuAc2Hex5HexNAc5dHex | 2570 | + | + | | | | | + | | | + | + | + | + | + |
| NeuAcHex5HexNAc5dHex3 | 2571 | | + | + | | | | + | | | + | + | + | + | + |
| NeuAcHex6HexNAc5dHex2 | 2587 | + | + | | + | | + | + | + | + | + | + | + | + | + |
| Hex7HexNAc6dHexSP | 2595 | | | | | | | + | | | | | | | |
| NeuAcHex7HexNAc5dHex and/or | 2603 | | + | | | + | + | + | + | | | + | + | | |
| NeuGcHex6HexNAc5dHex2 | | | | | | | | | | | | | | | |
| NeuAcHex8HexNAc5 and/or | 2619 | | | | | + | | | | | | + | + | | |
| NeuGcHex7HexNAc5dHex | | | | | | | | | | | | | | | |
| NeuGcHex8HexNAc5 and/or | 2635 | | | | | | | | | | | + | + | | |
| NeuAcHex4HexNAc5dHex4SP | | | | | | | | | | | | | | | |
| NeuAcHex6HexNAc6dHex | 2644 | | + | + | | | + | + | + | | + | + | + | + | + |
| NeuAc2Hex5HexNAc4dHex3 | 2659 | | | | | | | + | | | | + | | | |
| NeuGcNeuAc2Hex5HexNAc4dHex | 2674 | | | | | | + | + | | | | | | | |
| and/or NeuAc3Hex6HexNAc4 | | | | | | | | | | | | | | | |
| NeuAc2Hex4HexNAc5dHex2SP2 | 2714 | | | | | | | + | | | | + | + | + | |
| NeuAcHex4HexNAc5dHex4SP2 and/or | 2715 | | | | | | | | | | | | + | + | |
| NeuAc3Hex5HexNAc5 | | | | | | | | | | | | | | | |
| NeuAc2Hex5HexNAc5dHex2 | 2716 | | | | | | | | | | | | | + | |
| NeuAc2Hex6HexNAc5dHex | 2732 | + | + | + | + | | + | + | + | + | + | + | + | + | + |
| NeuAcHex6HexNAc5dHex3 | 2733 | + | + | | + | + | + | + | + | + | + | + | + | + | + |
| NeuGcNeuAcHex6HexNAc5dHex | 2748 | | | | | | | | | | | | + | | |
| NeuAcHex8HexNAc5dHex | 2765 | | | | | + | | | | | | | | | |
| NeuAcHex6HexNAc6dHex2 | 2791 | | | | | | | | | | | + | + | + | + |
| Hex6HexNAc6dHex3SP2 | 2805 | | | | | | | | | | | | | + | |
| NeuAcHex7HexNAc6dHex | 2807 | + | + | + | + | | + | + | + | + | + | + | + | + | + |
| NeuAc2Hex6HexNAc5dHexSP | 2812 | | | | | | | | + | | | + | + | + | + |
| NeuAcHex6HexNAc5dHex3SP | 2813 | | | | | | | | | | | | + | | |
| NeuAc3Hex6HexNAc4dHex and/or | 2820 | | | | | | | | | | | | + | | |
| NeuGcNeuAc2Hex5HexNAc4dHex2 | | | | | | | | | | | | | | | |
| NeuAc2Hex6HexNAc5dHex2 | 2879 | + | + | | + | + | + | + | + | + | + | + | + | + | + |
| NeuAcHex6HexNAc5dHex4 | 2880 | | | | | | + | | | | | + | + | + | + |
| NeuAc2Hex7HexNAc5dHex and/or | 2895 | | | | | + | | | | | | | + | | |
| NeuGcNeuAcHex6HexNAc5dHex2 | | | | | | | | | | | | | | | |
| NeuAc3Hex6HexNAc4dHexSP and/or | 2900 | | | | | | + | | | | | | | | |
| NeuGcNeuAc2Hex5HexNAc4dHex2SP | | | | | | | | | | | | | | | |
| NeuGc2Hex6HexNAc5dHex2 | 2911 | | | | | | | | | | | | | + | |
| NeuAc2Hex5HexNAc6dHex2 | 2920 | | | | | | | | | + | | | | | |
| NeuGcNeuAc2Hex5HexNAc6 | 2935 | | | | | | | | | | | + | | | |
| NeuAc2Hex6HexNAc6dHex and/or | 2936 | | + | + | | | | | | + | | + | + | + | + |
| NeuGcNeuAcHex5HexNAc6dHex2 | | | | | | | | | | | | | | | |
| NeuAcHex6HexNAc6dHex3 | 2937 | | | | | | | + | | | | | | + | |
| NeuGc2NeuAcHex5HexNAc6 and/or | 2951 | | | | | | | | + | | | | | | |
| NeuAc3Hex5HexNAc4dHex3 | | | | | | | | | | | | | | | |
| NeuAcHex7HexNAc6dHex2 | 2953 | + | | | + | | + | + | + | | | + | + | | + |
| Hex8HexNAc7dHexSP | 2961 | | | | | | | | | + | | | | | |
| NeuAc2Hex4HexNAc7dHex2 | 2961 | | | | | | | | | + | | | | | |
| NeuAcHex7HexNAc7dHex | 3010 | | + | | | | | | | | | | + | + | |
| NeuAc3Hex6HexNAc5dHex | 3024 | + | + | | + | | + | + | + | + | + | + | + | + | + |
| NeuAc2Hex6HexNAc5dHex3 | 3025 | + | + | | + | | + | + | | + | + | + | + | + | + |
| NeuGc3Hex6HexNAc5dHex and/or | 3072 | | | | | | | | | | | | + | | |
| NeuGc2NeuAcHex7HexNAc5 | | | | | | | | | | | | | | | |
| NeuAc2Hex6HexNAc6dHex2 | 3082 | | | | | | | | | | | | | + | |
| NeuAc2Hex7HexNAc6dHex | 3098 | + | + | + | + | | + | + | + | + | + | + | + | + | + |
| NeuAcHex7HexNAc6dHex3 | 3099 | + | + | | + | | + | + | + | + | + | + | + | + | + |
| NeuAc3Hex6HexNAc5dHexSP | 3104 | | | | | | | | | | | + | | + | |
| NeuAc2Hex6HexNAc5dHex3SP | 3105 | | | | | | | | | | | + | + | | |
| NeuAc3Hex6HexNAc5dHex2 | 3170 | | | | | | | | | | | + | + | | |
| NeuAc2Hex6HexNAc5dHex4 | 3171 | | + | | + | | + | + | | | | | + | + | |
| NeuAcHex8HexNAc7dHex | 3172 | + | | | + | | + | + | + | + | + | + | + | + | + |
| NeuAc3Hex6HexNAc6dHex | 3227 | | | | | | | | | | | + | | + | |
| NeuAc2Hex6HexNAc6dHex3 | 3228 | | | | | | | | | | | | | + | |
| NeuAc2Hex7HexNAc6dHex2 | 3244 | | | | | | | | + | + | | + | + | + | |
| NeuAcHex7HexNAc6dHex4 | 3245 | + | | | | | + | | + | | | + | + | + | |
| NeuAc2Hex7HexNAc7dHex | 3301 | | | | | | | | | | | | | + | |
| NeuAcHex7HexNAc7dHex3 | 3302 | | | | | | | | | | | | + | | |
| NeuAcHex8HexNAc7dHex2 | 3318 | | | | | | | | + | | | + | + | | |
| NeuAc3Hex7HexNAc6dHex | 3389 | | | | | | + | + | + | + | | + | + | + | |
| NeuAc2Hex7HexNAc6dHex3 | 3390 | + | + | | | | + | + | + | + | | + | + | + | + |
| NeuAcHex7HexNAc6dHex5 and/or | 3391 | | | | + | | + | | | | | | + | | |
| NeuAcHex9HexNAc8 | | | | | | | | | | | | | | | |
| NeuAc2Hex8HexNAc7dHex | 3463 | + | | | + | | + | + | + | + | | + | + | | + |
| NeuAcHex8HexNAc7dHex3 | 3464 | | | | | | + | | + | + | | + | + | | + |
| NeuAc2Hex7HexNAc6dHex4 | 3536 | | | | | | + | + | | | | + | + | + | + |
| NeuAcHex9HexNAc8dHex | 3537 | | | | + | | + | + | + | | | | + | | |
| NeuAc2Hex8HexNAc7dHex2 | 3609 | | | | | | | + | + | | | + | | | |
| NeuAcHex8HexNAc7dHex4 | 3610 | + | | | | | | | + | | | + | + | | |
| NeuAc4Hex7HexNAc6dHex | 3680 | | | | | | | | + | | | + | | + | |
| NeuAc3Hex7HexNAc6dHex3 | 3681 | | | | + | | + | | + | + | | + | + | + | |
| NeuAcHex9HexNAc8dHex2 | 3683 | | | | | | | + | | | | + | + | | |
| NeuAc3Hex8HexNAc7dHex | 3754 | | | | | | + | + | + | | | | | | + |
| NeuAc2Hex8HexNAc7dHex3 | 3755 | | | | + | | | | + | + | | + | + | + | |
| NeuAcHex10HexNAc9 and/or | 3756 | + | | | | | | | + | + | | | + | | |
| NeuAcHex8HexNAc7dHex5 | | | | | | | | | | | | | | | |
| NeuAc3Hex7HexNAc6dHex4 | 3827 | | | | | | + | | | | | | | | + |
| NeuAc2Hex9HexNAc8dHex | 3828 | | | | + | | | + | + | | | + | | | |
| NeuAcHex9HexNAc8dHex3 | 3829 | | | | | | | + | + | | | + | + | | |
| NeuAc2Hex8HexNAc7dHex4 | 3901 | | | | | | | + | | | | + | | + | |
| NeuAc2Hex9HexNAc8dHex2 | 3974 | | | | | | | | | | | + | | + | |
| NeuAcHex9HexNAc8dHex4 | 3975 | | | | | | | | | | | + | + | | |
| NeuAc4Hex8HexNAc7dHex | 4045 | | | | | | | | | | | | | + | |
| NeuAc3Hex8HexNAc7dHex3 | 4046 | | | | | | | | | | | | + | + | |
| NeuAc2Hex10HexNAc9 and/or | 4047 | | | | + | | | | + | | | | | | |
| NeuAc2Hex8HexNAc7dHex5 | | | | | | | | | | | | | | | |
| NeuAc3Hex9HexNAc8dHex | 4119 | | | | | | | | | | | | | + | |
| NeuAc2Hex9HexNAc8dHex3 | 4120 | | | | | | | | | | | | + | | |
| HexNAc ≧ 3 and dHex ≧ 1 | | | | | | | | | | | | | | | |
| (including multifucosylated N- | | | | | | | | | | | | | | | |
| glycans) | | | | | | | | | | | | | | | |
| Hex5HexNAc3dHex2SP | 1808 | | | | | | | | | | | | + | | |
| NeuAcHex4HexNAc3dHex2 | 1857 | | | | | | | | | | | + | + | | |
| NeuAcHex5HexNAc3dHex and/or | 1873 | + | + | + | + | + | + | + | + | + | + | + | + | + | + |
| NeuGcHex4HexNAc3dHex2 | | | | | | | | | | | | | | | |
| NeuAcHex4HexNAc3dHex3 | 2003 | | + | | | | + | | | | | | | | |
| NeuAcHex5HexNAc3dHex2 | 2019 | | | | | | | | | | | + | + | + | |
| Hex8HexNAc3dHexSP and/or | 2148 | | | | | | | | | | | | + | | |
| NeuAc2Hex4HexNAc3dHex2 | | | | | | | | | | | | | | | |
| NeuAc2Hex5HexNAc3dHex2 and/or | 2310 | | | | | | | | | + | | | | | |
| NeuGcNeuAcHex4HexNAc3dHex3 | | | | | | | | | | | | | | | |
| NeuAc2Hex5HexNAc3dHex2SP | 2390 | + | + | + | + | + | + | | | | + | + | + | + | |
| NeuAcHex8HexNAc3dHexSP and/or | 2439 | | | | | | | | | | | + | | | |
| NeuAc3Hex4HexNAc3dHex2 | | | | | | | | | | | | | | | |
| Hex5HexNAc4dHex2SP | 2011 | | | | | | | | | | | | + | | |
| Hex4HexNAc5dHex2SP | 2052 | + | + | | | | | + | + | | | | | | |
| NeuAcHex4HexNAc4dHex2 | 2060 | | | | | | + | | + | + | | + | + | + | |
| NeuAcHex3HexNAc5dHex2 and/or | 2101 | | | | | | | | | + | | | | | |
| NeuAc2Hex4HexNAc4Ac | | | | | | | | | | | | | | | |
| Hex4HexNAc5dHex2SP2 | 2132 | | | | + | | | | | | | | | | |
| Hex5HexNAc4dHex3SP | 2157 | | | | | | | | | | | | + | | |
| Hex6HexNAc4dHex2SP and/or | 2173 | | | | | | | | | | | | | + | |
| Hex3HexNAc6dHex2SP2 | | | | | | | | | | | | | | | |
| NeuAcHex5HexNAc4dHex2 | 2222 | + | + | + | + | + | + | + | + | + | + | + | + | + | + |
| NeuAcHex6HexNAc4dHex and/or | 2238 | + | + | | + | + | + | + | + | + | + | + | + | + | + |
| NeuGcHex5HexNAc4dHex2 | | | | | | | | | | | | | | | |
| NeuAcHex4HexNAc5dHex2 and/or | 2263 | + | | | | | | | | + | | | + | | |
| NeuAc2Hex5HexNAc4Ac | | | | | | | | | | | | | | | |
| NeuAcHex5HexNAc4dHex2SP | 2302 | | | | | | | | | | | | + | | |
| Hex6HexNAc4dHex3SP and/or | 2319 | | | | | | | | + | | | | + | + | |
| NeuGcNeuAcHex3HexNAc6 | | | | | | | | | | | | | | | |
| Hex7HexNAc4dHex2SP and/or | 2335 | | | | | | | | | | | | + | + | |
| Hex4HexNAc6dHex2SP2 | | | | | | | | | | | | | | | |
| NeuAcHex5HexNAc4dHex3 | 2368 | + | + | | + | + | + | + | + | + | + | + | + | + | + |
| NeuAcHex6HexNAc4dHex2 and/or | 2384 | + | + | | + | + | | | + | | | + | + | | |
| NeuGcHex5HexNAc4dHex3 | | | | | | | | | | | | | | | |
| NeuAc2Hex3HexNAc5dHex2 and/or | 2392 | | | | | + | + | | | | | | | | |
| Hex7HexNAc5dHexSP | | | | | | | | | | | | | | | |
| NeuAcHex3HexNAc5dHex4 | 2393 | | | | | + | | | | | | | | | |
| NeuAcHex4HexNAc6dHexSP and/or | 2400 | | | | | | | | | | | | + | | |
| NeuGcHex6HexNAc4dHex2 and/or | | | | | | | | | | | | | | | |
| NeuAcHex7HexNAc4dHex | | | | | | | | | | | | | | | |
| NeuAcHex4HexNAc5dHex3 and/or | 2409 | | | | | | | | + | | | | + | | |
| NeuAc2Hex5HexNAc4dHexAc | | | | | | | | | | | | | | | |
| NeuAcHex5HexNAc5dHex2 | 2425 | + | + | | | | | | + | + | + | + | + | + | + |
| NeuAcHex5HexNAc4dHex3SP | 2448 | | + | | | | | | | | | + | + | + | + |
| NeuAcHex4HexNAc5dHex3SP | 2489 | | | | | | | | | | | + | + | | |
| NeuAc2Hex5HexNAc4dHex2 | 2513 | | | | | | + | + | + | | | + | + | + | + |
| NeuAcHex5HexNAc4dHex4 | 2514 | | | | | | + | | | | | | | | + |
| NeuAcHex6HexNAc5dHexSP and/or | 2521 | + | + | | | | | + | | | | | + | | |
| NeuAc3Hex2HexNAc5dHex2 | | | | | | | | | | | | | | | |
| NeuAc2Hex6HexNAc4dHex and/or | 2529 | | + | | | | | | | | | + | + | + | |
| NeuGcNeuAcHex5HexNAc4dHex2 | | | | | | | | | | | | | | | |
| NeuGc2Hex5HexNAc4dHex2 and/or | 2545 | | | | | | | | | | | + | + | + | |
| NeuGcNeuAcHex6HexNAc4dHex | | | | | | | | | | | | | | | |
| NeuAcHex5HexNAc5dHex3 | 2571 | | + | + | | | | + | | | + | + | + | + | + |
| NeuAcHex6HexNAc5dHex2 | 2587 | + | + | | + | | + | + | + | + | + | + | + | + | + |
| NeuAcHex7HexNAc5dHex and/or | 2603 | | + | | | + | + | + | + | | | + | + | | |
| NeuGcHex6HexNAc5dHex2 | | | | | | | | | | | | | | | |
| NeuGcHex8HexNAc5 and/or | 2635 | | | | | | | | | | | + | + | | |
| NeuAcHex4HexNAc5dHex4SP | | | | | | | | | | | | | | | |
| NeuAc2Hex5HexNAc4dHex3 | 2659 | | | | | | | + | | | | + | | | |
| NeuGcNeuAc2Hex5HexNAc4dHex | 2674 | | | | | | + | + | | | | | | | |
| and/or NeuAc3Hex6HexNAc4 | | | | | | | | | | | | | | | |
| NeuAc2Hex4HexNAc5dHex2SP2 | 2714 | | | | | | | + | | | | + | + | + | |
| NeuAcHex4HexNAc5dHex4SP2 and/or | 2715 | | | | | | | | | | | | + | + | |
| NeuAc3Hex5HexNAc5 | | | | | | | | | | | | | | | |
| NeuAc2Hex5HexNAc5dHex2 | 2716 | | | | | | | | | | | | | + | |
| NeuAcHex6HexNAc5dHex3 | 2733 | + | + | | + | + | + | + | + | + | + | + | + | + | + |
| NeuAcHex6HexNAc6dHex2 | 2791 | | | | | | | | | | | + | + | + | + |
| Hex6HexNAc6dHex3SP2 | 2805 | | | | | | | | | | | | | + | |
| NeuAcHex6HexNAc5dHex3SP | 2813 | | | | | | | | | | | | + | | |
| NeuAc3Hex6HexNAc4dHex and/or | 2820 | | | | | | | | | | | | + | | |
| NeuGcNeuAc2Hex5HexNAc4dHex2 | | | | | | | | | | | | | | | |
| NeuAc2Hex6HexNAc5dHex2 | 2879 | + | + | | + | + | + | + | + | + | + | + | + | + | + |
| NeuAcHex6HexNAc5dHex4 | 2880 | | | | | | + | | | | | + | + | + | + |
| NeuAc2Hex7HexNAc5dHex and/or | 2895 | | | | | + | | | | | | | + | | |
| NeuGcNeuAcHex6HexNAc5dHex2 | | | | | | | | | | | | | | | |
| NeuAc3Hex6HexNAc4dHexSP and/or | 2900 | | | | | | + | | | | | | | | |
| NeuGcNeuAc2Hex5HexNAc4dHex2SP | | | | | | | | | | | | | | | |
| NeuGc2Hex6HexNAc5dHex2 | 2911 | | | | | | | | | | | | | + | |
| NeuAc2Hex5HexNAc6dHex2 | 2920 | | | | | | | | | + | | | | | |
| NeuAc2Hex6HexNAc6dHex and/or | 2936 | | + | + | | | | | | + | | + | + | + | + |
| NeuGcNeuAcHex5HexNAc6dHex2 | | | | | | | | | | | | | | | |
| NeuAcHex6HexNAc6dHex3 | 2937 | | | | | | | + | | | | | | + | |
| NeuGc2NeuAcHex5HexNAc6 and/or | 2951 | | | | | | | | + | | | | | | |
| NeuAc3Hex5HexNAc4dHex3 | | | | | | | | | | | | | | | |
| NeuAcHex7HexNAc6dHex2 | 2953 | + | | | + | | + | + | + | | | + | + | | + |
| NeuAc2Hex4HexNAc7dHex2 | 2961 | | | | | | | | | + | | | | | |
| NeuAc2Hex6HexNAc5dHex3 | 3025 | + | + | | + | | + | + | | + | + | + | + | + | + |
| NeuAc2Hex6HexNAc6dHex2 | 3082 | | | | | | | | | | | | | + | |
| NeuAcHex7HexNAc6dHex3 | 3099 | + | + | | + | | + | + | + | + | + | + | + | + | + |
| NeuAc2Hex6HexNAc5dHex3SP | 3105 | | | | | | | | | | | + | + | | |
| NeuAc3Hex6HexNAc5dHex2 | 3170 | | | | | | | | | | | + | + | | |
| NeuAc2Hex6HexNAc5dHex4 | 3171 | | + | | + | | + | + | | | | | + | + | |
| NeuAc2Hex6HexNAc6dHex3 | 3228 | | | | | | | | | | | | | + | |
| NeuAc2Hex7HexNAc6dHex2 | 3244 | | | | | | | | + | + | | + | + | + | |
| NeuAcHex7HexNAc6dHex4 | 3245 | + | | | | | + | | + | | | + | + | + | |
| NeuAcHex7HexNAc7dHex3 | 3302 | | | | | | | | | | | | + | | |
| NeuAcHex8HexNAc7dHex2 | 3318 | | | | | | | | + | | | + | + | | |
| NeuAc2Hex7HexNAc6dHex3 | 3390 | + | + | | | | + | + | + | + | | + | + | + | + |
| NeuAcHex7HexNAc6dHex5 and/or | 3391 | | | | + | | + | | | | | | + | | |
| NeuAcHex9HexNAc8 | | | | | | | | | | | | | | | |
| NeuAcHex8HexNAc7dHex3 | 3464 | | | | | | + | | + | + | | + | + | | + |
| NeuAc2Hex7HexNAc6dHex4 | 3536 | | | | | | + | + | | | | + | + | + | + |
| NeuAc2Hex8HexNac7dHex2 | 3609 | | | | | | | + | + | | | + | | | |
| NeuAcHex8HexNAc7dHex4 | 3610 | + | | | | | | | + | | | + | + | | |
| NeuAc3Hex7HexNAc6dHex3 | 3681 | | | | + | | + | | + | + | | + | + | + | |
| NeuAcHex9HexNAc8dHex2 | 3683 | | | | | | | + | | | | + | + | | |
| NeuAc2Hex8HexNAc7dHex3 | 3755 | | | | + | | | | + | + | | + | + | + | |
| NeuAcHex10HexNAc9 and/or | 3758 | + | | | | | | | + | + | | | + | | |
| NeuAcHex8HexNAc7dHex5 | | | | | | | | | | | | | | | |
| NeuAc3Hex7HexNAc6dHex4 | 3827 | | | | | | + | | | | | | | | + |
| NeuAcHex9HexNAc8dHex3 | 3829 | | | | | | | + | + | | | + | + | | |
| NeuAc2Hex8HexNAc7dHex4 | 3901 | | | | | | | + | | | | + | | + | |
| NeuAc2Hex9HexNAc8dHex2 | 3974 | | | | | | | | | | | + | | + | |
| NeuAcHex9HexNAc8dHex4 | 3975 | | | | | | | | | | | + | + | | |
| NeuAc3Hex8HexNAc7dHex3 | 4048 | | | | | | | | | | | | + | + | |
| NeuAc2Hex10HexNAc9 and/or | 4047 | | | | + | | | | + | | | | | | |
| NeuAc2Hex8HexNAc7dHex5 | | | | | | | | | | | | | | | |
| NeuAc2Hex9HexNAc8dHex3 | 4120 | | | | | | | | | | | | + | | |
| HexNAc > Hex ≧ 2 | | | | | | | | | | | | | | | |
| (terminal HexNAc, N > H) | | | | | | | | | | | | | | | |
| NeuAcHex3HexNAc4 | 1606 | | | | | | + | | | | | | | | |
| NeuAcHex3HexNAc4dHex | 1752 | | | | | | | | | | | | | | + |
| NeuAcHex3HexNac5 | 1809 | | | | | | | + | | | | | | | |
| NeuGcHex3HexNac5 | 1825 | | | | | | + | + | | | | | | | |
| Hex4HexNAc5dHexSP | 1906 | + | + | | | | | | | | | | | | |
| NeuAcHex4HexNAc5 | 1971 | + | + | | | + | + | | + | | | | + | | + |
| Hex7HexNAc4SP and/or | 2043 | | | | | | | + | | | | | | | |
| Hex4HexNAc6SP2 and/or | | | | | | | | | | | | | | | |
| NeuAc2Hex3HexNAc4dHex | | | | | | | | | | | | | | | |
| NeuAcHex4HexNAc5SP | 2051 | | + | | | | + | + | | | | | + | + | |
| Hex4HexNAc5dHex2SP | 2052 | + | + | | | | | + | + | | | | | | |
| NeuAcHex3HexNAc5dHex2 and/or | 2101 | | | | | | | | | + | | | | | |
| NeuAc2Hex4HexNAc4Ac | | | | | | | | | | | | | | | |
| NeuAcHex4HexNAc5dHex | 2117 | + | + | + | | + | | | | | + | + | + | + | + |
| Hex4HexNAc5dHex2SP2 | 2132 | | | | + | | | | | | | | | | |
| Hex6HexNAc4dHex2SP and/or | 2173 | | | | | | | | | | | | | + | |
| Hex3HexNAc6dHex2SP2 | | | | | | | | | | | | | | | |
| NeuAcHex4HexNAc6 | 2174 | + | | | | | | + | + | + | | | + | + | |
| NeuAc3Hex3HexNAc4 and/or | 2188 | | | | | | | + | | | | | + | | |
| NeuGcHex6HexNAc4SP and/or | | | | | | | | | | | | | | | |
| NeuAc2NeuGcHex2HexNAc4dHex | | | | | | | | | | | | | | | |
| NeuAc2Hex3HexNAc4dNex2 and/or | 2189 | | | | | + | + | | | | | | | | |
| Hex7HexNAc4dHexSP and/or | | | | | | | | | | | | | | | |
| Hex4HexNAc6dHexSP2 | | | | | | | | | | | | | | | |
| NeuAc2Hex3HexNAc5dHex and/or | 2246 | | | | | | + | + | + | + | | | | | |
| Hex7HexNAc5SP | | | | | | | | | | | | | | | |
| NeuAc2Hex4HexNAc5 | 2262 | | | | | | | + | | | | | | | |
| NeuAcHex4HexNAc5dHex2 and/or | 2263 | + | | | | | | | | + | | | + | | |
| NeuAc2Hex5HexNAc4Ac | | | | | | | | | | | | | | | |
| Hex6HexNAc4dHex3SP and/or | 2319 | | | | | | | | + | | | | + | + | |
| NeuGcNeuAcHex3HexNAc6 | | | | | | | | | | | | | | | |
| NeuAcHex4HexNAc6dHex | 2320 | | | | | | | | | | | | + | + | |
| Hex7HexNAc4dHex2SP and/or | 2335 | | | | | | | | | | | | + | + | |
| Hex4HexNAc6dHex2SP2 | | | | | | | | | | | | | | | |
| NeuAcHex5HexNAc6 | 2336 | | | | | | | | | | | + | | + | |
| NeuAc2Hex3HexNAc5dHex2 and/or | 2392 | | | | | + | + | | | | | | | | |
| Hex7HexNAc5dHexSP | | | | | | | | | | | | | | | |
| NeuAcHex3HexNAc5dHex4 | 2393 | | | | | + | | | | | | | | | |
| NeuAcHex4HexNAc6dHexSP and/or | 2400 | | | | | | | | | | | | + | | |
| NeuGcHex6HexNAc4dHex2 and/or | | | | | | | | | | | | | | | |
| NeuAcHex7HexNAc4dHex | | | | | | | | | | | | | | | |
| NeuAc2Hex4HexNAc5dHex | 2408 | + | + | | | | | | | | | | | + | |
| NeuAcHex4HexNAc5dHex3 and/or | 2409 | | | | | | | + | | | | | + | | |
| NeuAc2Hex5HexNAc4dHexAc | | | | | | | | | | | | | | | |
| NeuAcHex5HexNAc6dHex | 2482 | | | | | | | | | | | | | + | |
| NeuAcHex4HexNAc5dHex3SP | 2489 | | | | | | | | | | | + | + | | |
| Hex6HexNAc7SP | 2490 | | | | | | | | | | | | + | | |
| NeuAcHex6HexNAc5dHexSP and/or | 2521 | + | + | | | | | + | | | | | + | | |
| NeuAc3Hex2HexNAc5dHex2 | | | | | | | | | | | | | | | |
| NeuAc2Hex5HexNAc6 | 2627 | | | | | | | | + | | | | | | |
| NeuGcHex8HexNAc5 and/or | 2635 | | | | | | | | | | | + | + | | |
| NeuAcHex4HexNAc5dHex4SP | | | | | | | | | | | | | | | |
| NeuAc2Hex4HexNAc5dHex2SP2 | 2714 | | | | | | | + | | | | + | + | + | |
| NeuAcHex4HexNAc5dHex4SP2 and/or | 2715 | | | | | | | | | | | | + | + | |
| NeuAc3Hex5HexNAc5 | | | | | | | | | | | | | | | |
| NeuGcNeuAc2Hex5HexNAc6 | 2935 | | | | | | | | | | | + | | | |
| NeuGc2NeuAcHex5HexNAc6 and/or | 2951 | | | | | | | | + | | | | | | |
| NeuAc3Hex5HexNAc4dHex3 | | | | | | | | | | | | | | | |
| NeuAc2Hex4HexNAc7dHex2 | 2961 | | | | | | | | | + | | | | | |
| HexNAc = Hex ≧ 5 | | | | | | | | | | | | | | | |
| (terminal HexNAc, N = H) | | | | | | | | | | | | | | | |
| Hex5HexNAc5SP2 | 2002 | | | | + | + | + | + | + | + | | + | | | |
| NeuAcHex5HexNAc5 | 2133 | + | + | | | + | + | | + | | + | + | + | + | + |
| NeuAcHex5HexNAc5dHex | 2279 | + | + | + | + | + | + | + | + | + | + | + | + | + | + |
| NeuAc2Hex5HexNAc5 | 2424 | | | | | | + | | | | + | + | | + | + |
| NeuAcHex5HexNAc5dHex2 | 2425 | + | + | | | | | + | + | + | + | + | + | + | + |
| NeuAc2Hex5HexNAc5dHex | 2570 | + | + | | | | | + | | | + | + | + | + | + |
| NeuAcHex5HexNAc5dHex3 | 2571 | | + | + | | | | + | | | + | + | + | + | + |
| NeuAcHex6HexNAc6dHex | 2644 | | + | + | | | + | + | + | | + | + | + | + | + |
| NeuAcHex4HexNAc5dHex4SP2 and/or | 2715 | | | | | | | | | | | | + | + | |
| NeuAc3Hex5HexNAc5 | | | | | | | | | | | | | | | |
| NeuAc2Hex5HexNAc5dHex2 | 2716 | | | | | | | | | | | | | + | |
| NeuAcHex6HexNAc6dHex2 | 2791 | | | | | | | | | | | + | + | + | + |
| Hex6HexNAc6dHex3SP2 | 2805 | | | | | | | | | | | | | + | |
| NeuAc2Hex6HexNAc6dHex and/or | 2936 | | + | + | | | | | | + | | + | + | + | + |
| NeuGcNeuAcHex5HexNAc6dHex2 | | | | | | | | | | | | | | | |
| NeuAcHex6HexNAc6dHex3 | 2937 | | | | | | | + | | | | | | + | |
| NeuAcHex7HexNAc7dHex | 3010 | | + | | | | | | | | | | + | + | |
| NeuAc3Hex6HexNAc6dHex | 3227 | | | | | | | | | | | + | | + | |
| NeuAc2Hex6HexNAc6dHex3 | 3228 | | | | | | | | | | | | | + | |
| NeuAc2Hex7HexNAc7dHex | 3301 | | | | | | | | | | | | | + | |
| NeuAcHex7HexNAc7dHex3 | 3302 | | | | | | | | | | | | + | | |
| SP ≧ 1 | | | | | | | | | | | | | | | |
| (including sulphated and/or | | | | | | | | | | | | | | | |
| phosphorylated glycans) | | | | | | | | | | | | | | | |
| Hex3HexNAc2SP | 989 | | | | | + | + | + | | | | | | | |
| Hex3HexNAc2dHexSP | 1135 | | | | | | | | | | | + | + | | |
| Hex4HexNAc2SP | 1151 | | | | | + | + | + | + | | | + | | | |
| Hex3HexNAc3SP | 1192 | | | | | | | + | | | | | | | |
| Hex5HexNAc2SP | 1313 | | | | | | | | | | | + | | | |
| Hex3HexNAc3dHexSP | 1338 | | | | | | | + | | | | | | | |
| Hex4HexNAc3SP | 1354 | | | | | | + | + | | | | | | | |
| Hex6HexNAc2SP | 1475 | | + | | + | | + | + | | + | | + | + | + | |
| Hex4HexNAc3dHexSP | 1500 | + | + | | | + | + | + | + | | | + | + | + | + |
| Hex5HexNAc3SP | 1516 | | | | | | + | + | | | | | | | + |
| Hex8HexNAc2SP2 | 1555 | | | | | | | | | | | | | + | |
| Hex4HexNAc4SP | 1557 | | + | | | + | | + | + | | | | | | |
| NeuAcHex3HexNAc3SP2 | 1563 | | | | | | | | | | | + | + | | |
| Hex4HexNAc4SP2 and/or | 1637 | | | | + | | + | + | | | | + | + | + | + |
| Hex7HexNAc2SP | | | | | | | | | | | | | | | |
| Hex4HexNAc3dHex2SP | 1646 | | | | | | | + | | | | + | | | |
| Hex5HexNAc3dHexSP | 1662 | | | | | | | + | | | | | | | |
| Hex6HexNAc3SP | 1678 | + | + | | + | + | + | + | + | + | | + | + | + | |
| Hex4HexNAc4dHexSP | 1703 | | + | | | + | | + | | | | | | | |
| NeuAcHex3HexNAc3dHexSP2 | 1709 | | | | | | | | | | | + | + | | |
| Hex4HexNAc4SP3 and/or | 1717 | | | | | | | | | | | | | + | |
| Hex7HexNAc2SP2 | | | | | | | | | | | | | | | |
| Hex5HexNAc4SP | 1719 | | + | | + | + | + | + | + | | | | | | |
| Hex7HexNAc2dHexSP | 1783 | | | | | | | | | | | | | + | |
| NeuAcHex4HexNAc3dHexSP | 1791 | | + | | | | + | + | + | | | | + | + | |
| Hex5HexNAc4SP2 and/or | 1799 | | | | | | + | | | | | | + | | |
| Hex8HexNAc2SP | | | | | | | | | | | | | | | |
| Hex5HexNAc3dHex2SP | 1808 | | | | | | | | | | | + | | | |
| NeuAc2Hex5HexNAc2 and/or | 1815 | | | | | | | | | | | + | | | |
| NeuAc2Hex2HexNAc4SP | | | | | | | | | | | | | | | |
| Hex5HexNAc4dHexSP | 1865 | + | + | | + | + | + | + | + | | | + | + | + | + |
| Hex6HexNAc4SP | 1881 | | | | | + | | | | | | | | | |
| Hex4HexNAc5dHexSP | 1906 | + | + | | | | | | | | | | | | |
| NeuAcHex6HexNAc2dHexSP and/or | 1912 | | | | | | | | | | | | + | | |
| NeuAcHex3HexNAc4dHexSP2 | | | | | | | | | | | | | | | |
| NeuAcHex4HexNAc4SP2 | 1928 | | | | | | | | | | | + | + | | |
| Hex8HexNAc3SP and/or | 2002 | | | | + | + | + | + | + | + | | + | | | + |
| Hex5HexNAc5SP2 and/or | | | | | | | | | | | | | | | |
| NeuAc2Hex4HexNAc3dHex | | | | | | | | | | | | | | | |
| NeuAcHex5HexNAc4SP | 2010 | | | | | | | + | | | | | + | | |
| Hex5HexNAc4dHex2SP | 2011 | | | | | | | | | | | | + | | |
| NeuGcHex5HexNAc4SP | 2026 | | | | | | | | | | | | + | | |
| Hex6HexNAc4dHexSP | 2027 | | | | | + | | | | | | | + | | |
| Hex7HexNAc4SP and/or | 2043 | | | | | | | + | | | | | | | |
| Hex4HexNAc6SP2 and/or | | | | | | | | | | | | | | | |
| NeuAc2Hex3HexNAc4dHex | | | | | | | | | | | | | | | |
| NeuAcHex7HexNAc3 and/or | 2051 | | + | | | | + | + | | | + | | + | + | + |
| NeuAcHex4HexNAc5SP | | | | | | | | | | | | | | | |
| Hex4HexNAc5dHex2SP | 2052 | + | + | | | | | + | + | | | | | | |
| NeuAcHex4HexNAc4dHexSP2 | 2074 | | | | | | | | | | | + | + | | |
| NeuAc2Hex4HexNAc3dHexSP and/or | 2082 | | | | | | + | + | + | | | | | | |
| Hex8HexNAc3SP2 and/or | | | | | | | | | | | | | | | |
| Hex5HexNAc5SP3 | | | | | | | | | | | | | | | |
| NeuAcHex6HexNAc3dHexSP | 2115 | | | | | | | | | | | | + | | |
| Hex7HexNAc3dHex2SP and/or | 2132 | | | | + | | | | | | | | | | |
| NeuAc2Hex3HexNAc3dHex3 and/or | | | | | | | | | | | | | | | |
| Hex4HexNAc5dHex2SP2 | | | | | | | | | | | | | | | |
| Hex8HexNAc3dHexSP and/or | 2148 | | | | | | | | | | | | + | | |
| NeuAc2Hex4HexNAc3dHex2 | | | | | | | | | | | | | | | |
| NeuAcHex5HexNAc4dHexSP and/or | 2156 | + | + | | | | | + | + | | | | + | + | + |
| NeuAcHex8HexNAc2dHex | | | | | | | | | | | | | | | |
| Hex5HexNAc4dHex3SP | 2157 | | | | | | | | | | | | + | | |
| NeuAc2Hex5HexNAc3dHex and/or | 2164 | + | + | | | | + | | | | | | | | |
| Hex6HexNAc5SP2 | | | | | | | | | | | | | | | |
| NeuAc2Hex4HexNAc4SP2 | 2219 | | | | | | | | | | | + | | | |
| Hex6HexNAc5dHexSP | 2230 | | + | | + | | | + | + | | | | | | |
| NeuAc2Hex3HexNAc5dHex and/or | 2246 | | | | | | + | + | + | + | | | | | |
| Hex7HexNAc5SP | | | | | | | | | | | | | | | |
| NeuAc2Hex4HexNAc4dHexSP and/or | 2285 | | | | | | | | | | | | | + | |
| Hex11HexNAc2SP | | | | | | | | | | | | | | | |
| NeuAcHex8HexNAc3SP and/or | 2293 | | | | | | | | | | | | + | | |
| NeuAc3Hex4HexNAc3dHex | | | | | | | | | | | | | | | |
| NeuAc2Hex5HexNAc4SP | 2301 | | | | | | | | | | | | + | | |
| NeuAcHex5HexNAc4dHex2SP | 2302 | | | | | | | | | | | | + | | |
| Hex6HexNAc4dHex3SP | 2319 | | | | | | | | + | | | | | | |
| Hex7HexNAc4dHex2SP and/or | 2335 | | | | | | | | | | | | + | + | |
| Hex4HexNAc6dHex2SP2 | | | | | | | | | | | | | | | |
| NeuAc2Hex4HexNAc4dHexSP | 2365 | | | | | | | | | | | + | + | + | |
| NeuAc3Hex5HexNAc3SP and/or | 2389 | | | | | + | | | | | | | | | |
| NeuAc2Hex5HexNAc4Ac4 | | | | | | | | | | | | | | | |
| NeuAc2Hex5HexNAc3dHex2SP | 2390 | + | + | + | + | + | + | | | | + | + | | + | |
| NeuAc2Hex3HexNAc5dHex2 and/or | 2392 | | | | | + | + | | | | | | | | |
| Hex7HexNAc5dHexSP | | | | | | | | | | | | | | | |
| NeuAcHex4HexNAc6dHexSP and/or | 2400 | | | | | | | | | | | | + | | |
| NeuGcHex6HexNAc4dHex2 and/or | | | | | | | | | | | | | | | |
| NeuAcHex7HexNAc4dHex | | | | | | | | | | | | | | | |
| NeuAc2Hex6HexNAc3dHexSP | 2406 | | | | | + | | | | | | + | + | | |
| NeuAcHex8HexNAc3dHexSP and/or | 2439 | | | | | | | | | | | + | | | |
| NeuAc3Hex4HexNAc3dHex2 | | | | | | | | | | | | | | | |
| NeuAc2Hex5HexNAc4dHexSP and/or | 2447 | | + | | | | | + | + | | | + | + | + | + |
| NeuAc2Hex8HexNAc2dHex and/or | | | | | | | | | | | | | | | |
| Hex12HexNAc2SP | | | | | | | | | | | | | | | |
| NeuAcHex5HexNAc4dHex3SP and/or | 2448 | | + | | | | | | | | | + | + | + | + |
| NeuAcHex8HexNAc2dHex3 | | | | | | | | | | | | | | | |
| NeuAcHex7HexNAc3dHex3 and/or | 2489 | | | | | | | | | | | + | + | | |
| NeuAcHex4HexNAc5dHex3SP | | | | | | | | | | | | | | | |
| Hex6HexNAc7SP | 2490 | | | | | | | | | | | | + | | |
| NeuAcHex6HexNAc5dHexSP and/or | 2521 | + | + | | | | | + | | | | | + | | |
| NeuAcHex9HexNAc3dHex and/or | | | | | | | | | | | | | | | |
| NeuAc3Hex2HexNAc5dHex2 | | | | | | | | | | | | | | | |
| Hex6HexNAc5dHex3SP | 2522 | | | | | | | + | | | | | + | | |
| Hex7HexNAc6dHexSP | 2595 | | | | | | | + | | | | | | | |
| NeuGcHex8HexNAc5 and/or | 2635 | | | | | | | | | | | + | + | | |
| NeuAcHex4HexNAc5dHex4SP | | | | | | | | | | | | | | | |
| NeuAc2Hex4HexNAc5dHex2SP2 | 2714 | | | | | | | + | | | | + | + | + | |
| NeuAcHex4HexNAc5dHex4SP2 and/or | 2715 | | | | | | | | | | | | + | + | |
| NeuAc3Hex5HexNAc5 | | | | | | | | | | | | | | | |
| NeuAc3Hex5HexNAc4dHex2 and/or | 2804 | | | | | | | | | | | | + | + | |
| NeuAcHex6HexNAc6dHexSP2 | | | | | | | | | | | | | | | |
| Hex6HexNAc6dHex3SP2 | 2805 | | | | | | | | | | | | | + | |
| NeuAc2Hex6HexNAc5dHexSP | 2812 | | | | | | | | + | | | + | + | + | + |
| NeuAcHex6HexNAc5dHex3SP | 2813 | | | | | | | | | | | | + | | |
| NeuAc3Hex6HexNAc4dHexSP and/or | 2900 | | | | | | + | | | | | | | | |
| NeuGcNeuAc2Hex5HexNAc4dHex2SP | | | | | | | | | | | | | | | |
| NeuAc3Hex6HexNAc5dHexSP | 3104 | | | | | | | | | | | + | | + | |
| NeuAc2Hex6HexNAc5dHex3SP | 3105 | | | | | | | | | | | + | + | | |

hESC, human embryonic stem cells;
EB, embryoid bodies derived from hESC;
st.3, stage 3 differentiated cells derived from hESC;
hEF, human fibroblast feeder cells;
mEF, murine fibroblast feeder cells;
BM MSC, bone-marrow derived mesenchymal stem cells;
OB, Osteoblast-differentiated cells derived from BM MSC;
CB MSC, cord blood derived mesenchymal stem cells;
OB, adipocyte-differentiated cells derived from CB MSC;
CB MNC, cord blood mononuclear cells;
CD34+, CD133+, LIN−, and CD8−: subpopulations of CB MNC.

TABLE 7
Characteristic N-glycan signals of hESC.
Neutral N-glycans:

| | m/z | Proposed | |
|---|---|---|---|
| No. | [M + Na]⁺ | composition | Proposed classification |
| 1. | 1905.6 | H9N2 | high-mannose |
| 2. | 1419.5 | H6N2 | high-mannose |
| 3. | 1743.6 | H8N2 | high-mannose |
| 4. | 1257.4 | H5N2 | high-mannose |
| 5. | 1581.5 | H7N2 | high-mannose |
| 6. | 1079.4 | H3N2F1 | low-mannose |
| 7. | 2067.7 | H10N2 | other types (glucosylated) |
| 8. | 1095.4 | H4N2 | low-mannose |
| 9. | 933.3 | H3N2 | low-mannose |
| 10. | 1663.6 | H5N4 | complex-type |
| 11. | 1622.6 | H6N3 | hybrid/monoantennary |
| 12. | 1809.6 | H5N4F1 | complex-type |
| 13. | 1460.5 | H5N3 | hybrid/monoantennary |
| 14. | 1485.5 | H3N4F1 | complex-type; terminal |
| | | | N-acetylhexosamine (N > H) |
| 15. | 1444.5 | H4N3F1 | hybrid/monoantennary |

Sialylated N-glycans:

| | m/z | Proposed | |
|---|---|---|---|
| No. | [M − H]⁻ | composition | Proposed classification |
| 1. | 2076.7 | S1H5N4F1 | complex-type |
| 2. | 2222.8 | S1H5N4F2 | complex-type; complex fucosylation |
| 3. | 2367.8 | S2H5N4F1 | complex-type |
| 4. | 1930.7 | S1H5N4 | complex-type |
| 5. | 2441.9 | S1H6N5F1 | complex-type |
| 6. | 2092.7 | G1H5N4F1 | complex-type |
| 7. | 2117.8 | S1H4N5F1 | complex-type; terminal |
| | | | N-acetylhexosamine (N > H) |
| 8. | 2587.9 | S1H6N5F2 | complex-type; complex fucosylation |
| 9. | 2368.9 | S1H5N4F3 | complex-type; complex fucosylation |
| 10. | 2263.8 | S1H4N5F2 | complex-type; complex fucosylation; |
| | | | terminal N-acetylhexosamine(N > H) |
| 11. | 1711.6 | S1H4N3F1 | hybrid/monoantennary |
| 12. | 2279.8 | S1H5N5F1 | complex-type; terminal |
| | | | N-acetylhexosamine (N═H ≧ 5) |
| 13. | 2238.8 | G1H5N4F2 | complex-type; complex fucosylation |
| 14. | 2733.0 | S2H6N5F1 | complex-type |
| 15. | 2807.0 | S1H7N6F1 | complex-type |

The 15 characteristic neutral (upper panel) and sialylated (lower panel) N-glycan signals of the hESC N-glycome. The signals are expressed in all the analyzed hESC samples and they are listed in order of relative abundance (No) in each N-glycan fraction.
H: hexose,
N: N-acetylhexosamine,
F: deoxyhexose,
S: N-acetylneuraminic acid,
G: N-glycolylneuraminic acid. The proposed structural classification is according to FIG. 3A and as described in the text.

TABLE 8
NMR analysis of the major neutral N-glycans of hESC.

| Glycan residue | | | ¹H-NMR chemical shift (ppm) | | | | |
|---|---|---|---|---|---|---|---|
| Residue | Linkage | Proton | A | B | C | D | hESC ¹⁾ |
| D-GlcNAc | | H-1α | 5.191 | 5.187 | 5.187 | 5.188 | 5.188 |
| | | H-1β | 4.690 | 4.693 | 4.693 | 4.695 | 4.694 |
| | | NAc | 2.042 | 2.037 | 2.037 | 2.038 | 2.038 |
| β-D- | 4 | H-1 | 4.596 | 4.586 | 4.586 | 4.600 | 4.596 |
| GlcNAc | | NAc | 2.072 | 2.063 | 2.063 | 2.064 | 2.061 |
| β-D- | 4,4 | H-1 | 4.775 | 4.771 | 4.771 | 4.780 | ²⁾ |
| Man | | H-2 | 4.238 | 4.234 | 4.234 | 4.240 | 4.234 |
| α-D- | 6,4,4 | H-1 | 4.869 | 4.870 | 4.870 | 4.870 | 4.869 |
| Man | | H-2 | 4.149 | 4.149 | 4.149 | 4.150 | 4.153 |
| α-D- | 6,6,4,4 | H-1 | 5.153 | 5.151 | 5.151 | 5.143 | 5.148 |
| Man | | H-2 | 4.025 | 4.021 | 4.021 | 4.020 | 4.023 |
| α-D- | 2,6,6,4,4 | H-1 | 5.047 | 5.042 | 5.042 | 5.041 | 5.042 |
| Man | | H-2 | 4.074 | 4.069 | 4.069 | 4.070 | 4.069 |
| α-D- | 3,6,4,4 | H-1 | 5.414 | 5.085 | 5.415 | 5.092 | 5.408/5.085 |
| Man | | H-2 | 4.108 | 4.069 | 4.099 | 4.070 | 4.102/4.069 |
| α-D- | 2,3,6,4,4 | H-1 | 5.047 | — | 5.042 | — | 5.042 |
| Man | | H-2 | 4.074 | — | 4.069 | — | 4.069 |
| α-D- | 3,4,4 | H-1 | 5.343 | 5.341 | 5.341 | 5.345 | 5.346/5.338 |
| Man | | H-2 | 4.108 | 4.099 | 4.099 | 4.120 | 4.102 |
| α-D- | 2,3,4,4 | H-1 | 5.317 | 5.309 | 5.050 | 5.055 | 5.301/5.057 |
| Man | | H-2 | 4.108 | 4.099 | 4.069 | 4.070 | 4.102/4.069 |
| α-D- | 2,2,3,4,4 | H-1 | 5.047 | 5.042 | — | — | 5.042 |
| Man | | H-2 | 4.074 | 4.069 | — | — | 4.069 |

¹⁾ Chemical shifts determined from the center of the signal.
²⁾ Signal under HDO.
The identified signals were consistent with high-mannose type N-glycan
structures such as the structures A-D that have monosaccharide
compositions H₇₋₉N₂. The significant signals in the NMR spectrum can be
explained by the following glycan structure combinations: A + B + C + D,
A + B + D, A + C + D, B + C + D, A + D, or B + C. Reference data is
after Fu et al. (Fu, D., et al., 1994, Carbohydr. Res. 261, 173-186) and
Hård et al. (Hård, K., et al., Glycoconj. J. 8, 17-28). Monosaccharide
symbols are as in Supplementary FIG. S1.
A

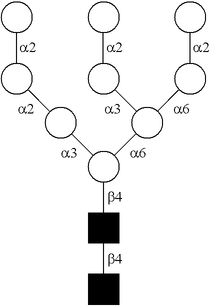

B

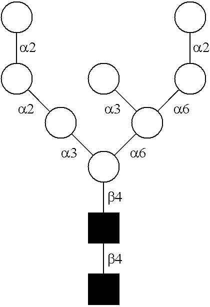

C

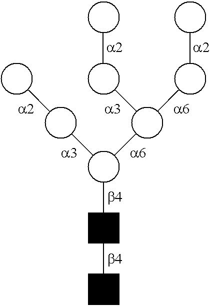

D

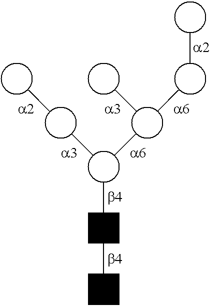

TABLE 9
NMR analysis of the major sialylated
N-glycan core structures of hESC.

| Glycan residue | | | ¹H-NMR chemical shift (ppm) | | | | |
|---|---|---|---|---|---|---|---|
| Residue | Linkage | Proton | A | B | C | D | hESC ¹⁾ |
| D-GlcNAc | | H-1α | 5.188 | 5.189 | 5.181 | 5.189 | 5.182/5.188 |
| | | NAc | 2.038 | 2.038 | 2.039 | 2.038 | 2.038 |
| α-L- | 6 | H-1α | — | — | 4.892 | — | 4.893 |
| Fuc | | H-1β | — | — | 4.900 | — | 4.893 |
| | | CH₃α | — | — | 1.211 | — | 1.210 |
| | | CH₃β | — | — | 1.223 | — | 1.219 |
| β-D- | 4 | H-1β | 4.604 | 4.606 | n.a. | 4.604 | 4.605 |
| GlcNAc | | NAc | 2.081 | 2.081 | 2.096 | 2.084 | 2.081/2.095 |
| β-D- | 4,4 | H-1 | n.a. | n.a. | n.a. | n.a. | n.a. |
| Man | | H-2 | 4.246 | 4.253 | 4.248 | 4.258 | 4.256 |
| α-D- | 6,4,4 | H-1 | 4.928 | 4.930 | 4.922 | 4.948 | 4.927 |
| Man | | H-2 | 4.11 | 4.112 | 4.11 | 4.117 | n.a. |
| β-D- | 2,6,4,4 | H-1 | 4.581 | 4.582 | 4.573 | 4.604 | 4.579/4.605 |
| GlcNAc | | NAc | 2.047 | 2.047 | 2.043 | 2.066 | 2.047/2.069 |
| β-D-Gal | 4,2,6,4,4 | H-1 | 4.473 | 4.473 | 4.550 | 4.447 | 4.447/4.472/ |
| | | | | | | | 4.545 |
| | | H-4 | n.a. | n.a. | n.a. | n.a. | 4.185 |
| α-D- | 3,4,4 | H-1 | 5.118 | 5.135 | 5.116 | 5.133 | 5.118/5.134 |
| Man | | H-2 | 4.190 | 4.196 | 4.189 | 4.197 | 4.195 |
| β-D- | 2,3,4,4 | H-1 | 4.573 | 4.606 | 4.573 | 4.604 | 4.579/4.605 |
| GlcNAc | | NAc | 2.047 | 2.069 | 2.048 | 2.070 | 2.047/2.069 |
| β-D-Gal | 4,2,3,4,4 | H-1 | 4.545 | 4.445 | 4.544 | 4.443 | 4.445/4.545 |
| | | H-3 | 4.113 | n.a. | 4.113 | n.a. | n.a. |

¹⁾ Chemical shifts determined from the center of the signal.
n.a.: Not assigned.
The identified signals were consistent with sialylated biantennary
complex-type N-glycan structures such as the structures A-D that have
monosaccharide compositions S₁₋₂H₅N₄F₀₋₁. Reference data is after Hård
et al. (Hård, K., et al., 1992, Eur. J. Biochem. 209, 895-915) and Helin et
al. (Helin, J., et al., 1995, Carbohydr. Res. 266, 191-209). The significant
signals in the NMR spectrum can be explained by the structural
components of these reference structures (not shown). Monosaccharide
symbols are as in Supplementary FIG. S1.
A

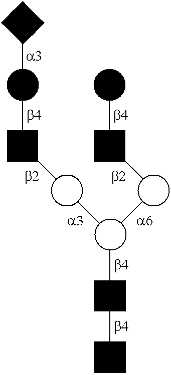

B

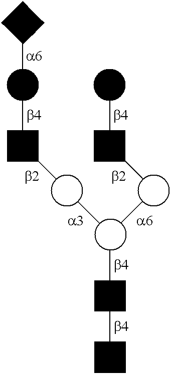

C

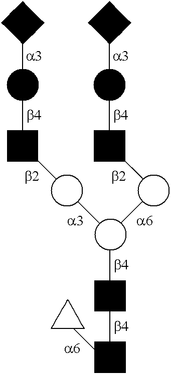

D

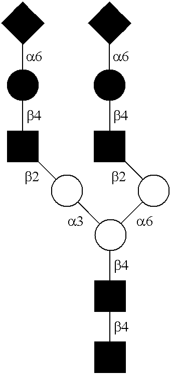

TABLE 10
Relative proportions (%) of sialylated N-glycan signals in hESC and differentiated cell lines.

| Proposed | | | | | | | | | | | | | |
|---|---|---|---|---|---|---|---|---|---|---|---|---|---|
| composition | m/z | FES 21 | EB 21 | St.3 21 | FES 22 | EB 22 | St.3 22 | FES 30 | EB 30 | St.3 30 | FES 29 | EB 29 | St.3 29 |
| S1H4N3F1 | 1711 | 2.16 | 2.68 | 2.73 | 2.25 | 3.02 | 3.46 | 1.77 | 3.16 | 3.05 | 1.86 | 2.41 | 2.89 |
| S1H6N3 | 1889 | 1.44 | 2.17 | 3.05 | 0.00 | 1.64 | 2.53 | 1.74 | 2.18 | 2.45 | 0.96 | 2.59 | 0.93 |
| S1H5N3 | 1727 | 1.54 | 1.48 | 1.86 | 0.00 | 1.36 | 3.15 | 0.99 | 1.06 | 1.71 | 1.07 | 2.39 | 0.79 |
| S1H4N3 | 1565 | 1.13 | 1.13 | 1.19 | 0.00 | 1.27 | 1.52 | 0.93 | 0.99 | 1.50 | 0.76 | 0.69 | 0.00 |
| S1H5N3F1 | 1873 | 0.81 | 2.26 | 3.13 | 0.00 | 1.46 | 2.14 | 1.42 | 1.68 | 1.86 | 0.00 | 2.17 | 1.31 |
| S2H5N3F1 | 2164 | 0.00 | 0.61 | 1.64 | 0.00 | 0.59 | 0.00 | 0.00 | 0.56 | 0.00 | 0.96 | 0.00 | 0.00 |
| S1H6N3F1 | 2035 | 0.00 | 1.28 | 1.23 | 0.00 | 0.66 | 2.05 | 0.00 | 0.71 | 1.08 | 0.00 | 0.66 | 0.71 |
| S1H5N4F1 | 2076 | 28.66 | 28.27 | 18.93 | 26.02 | 30.38 | 15.78 | 27.66 | 25.28 | 26.15 | 25.91 | 23.90 | 21.83 |
| S1H5N4F2 | 2222 | 12.84 | 3.35 | 3.98 | 15.53 | 2.83 | 2.19 | 10.12 | 5.19 | 2.62 | 9.18 | 3.21 | 1.61 |
| S2H5N4F1 | 2367 | 5.89 | 4.52 | 2.88 | 9.69 | 3.74 | 2.40 | 7.73 | 4.22 | 3.55 | 7.22 | 4.95 | 7.08 |
| S1H5N4 | 1930 | 5.55 | 5.53 | 5.03 | 4.30 | 4.91 | 3.37 | 6.13 | 4.70 | 5.57 | 6.18 | 4.89 | 3.76 |
| S1H6N5F1 | 2441 | 5.06 | 3.13 | 3.70 | 5.85 | 3.86 | 4.13 | 3.97 | 4.28 | 4.39 | 4.07 | 3.31 | 4.82 |
| G1H5N4F1 | 2092 | 3.61 | 3.10 | 0.00 | 2.81 | 2.56 | 0.00 | 5.00 | 2.85 | 0.00 | 4.87 | 1.89 | 0.00 |
| S1H4N5F1 | 2117 | 3.69 | 5.33 | 3.62 | 3.27 | 4.17 | 4.20 | 2.27 | 4.64 | 3.14 | 2.12 | 4.74 | 4.81 |
| S1H6N5F2 | 2587 | 2.67 | 0.70 | 1.51 | 4.06 | 0.66 | 0.00 | 1.95 | 1.07 | 1.28 | 2.25 | 1.13 | 1.09 |
| S1H5N4F3 | 2368 | 1.91 | 1.62 | 1.08 | 3.57 | 1.01 | 0.13 | 1.14 | 0.73 | 1.47 | 3.16 | 2.81 | 0.82 |
| S1H4N5F2 | 2263 | 4.17 | 1.33 | 1.27 | 2.44 | 1.00 | 2.91 | 1.24 | 2.15 | 0.98 | 1.72 | 1.35 | 1.08 |
| S1H5N5F1 | 2279 | 1.96 | 7.31 | 11.76 | 2.38 | 12.21 | 13.72 | 1.53 | 7.97 | 11.61 | 1.73 | 9.91 | 14.65 |
| S2H6N5F1 | 2732 | 1.56 | 0.82 | 1.36 | 2.18 | 0.80 | 0.00 | 1.16 | 0.35 | 1.25 | 1.46 | 0.28 | 2.21 |
| S1H6N4F1 | 2238 | 1.44 | 1.06 | 1.69 | 2.82 | 0.79 | 1.46 | 1.56 | 2.57 | 2.00 | 0.00 | 0.69 | 1.02 |
| S1G1H5N4 | 2237 | 1.05 | 0.56 | 0.00 | 0.00 | 0.77 | 0.00 | 2.23 | 1.12 | 0.00 | 2.22 | 1.66 | 0.00 |
| S1H7N6F1 | 2807 | 1.42 | 0.47 | 0.00 | 2.26 | 0.47 | 1.23 | 0.70 | 0.95 | 1.86 | 1.03 | 1.13 | 1.70 |
| S1H7N6F3 | 3099 | 0.68 | 0.00 | 0.00 | 1.98 | 0.00 | 0.00 | 0.45 | 0.06 | 0.57 | 1.84 | 0.00 | 0.00 |
| S2H4N5F1 | 2408 | 1.72 | 0.77 | 0.00 | 2.23 | 0.43 | 0.00 | 0.00 | 0.72 | 0.00 | 0.94 | 0.00 | 0.00 |
| S1H5N5F2 | 2425 | 1.00 | 1.60 | 1.78 | 2.01 | 1.20 | 2.09 | 0.83 | 1.90 | 1.85 | 1.04 | 1.77 | 1.59 |
| S2H5N4 | 2221 | 0.00 | 1.48 | 0.00 | 0.08 | 1.42 | 1.31 | 2.14 | 1.70 | 1.39 | 2.62 | 2.13 | 4.35 |
| G2H5N4 | 2253 | 0.00 | 0.00 | 0.00 | 0.00 | 0.52 | 0.00 | 2.37 | 1.13 | 0.00 | 2.01 | 0.28 | 0.00 |
| G1H5N4 | 1946 | 1.21 | 1.28 | 0.00 | 0.00 | 0.00 | 0.00 | 1.28 | 0.57 | 0.00 | 1.68 | 0.00 | 0.00 |
| S1H6N4F2 | 2384 | 0.00 | 0.93 | 1.13 | 0.00 | 0.31 | 0.00 | 2.64 | 0.91 | 0.00 | 1.34 | 0.00 | 0.00 |
| S1H6N5 | 2295 | 1.26 | 1.03 | 1.73 | 0.00 | 1.22 | 0.00 | 1.21 | 1.00 | 0.69 | 1.10 | 1.09 | 0.00 |
| S1H6N5F3 | 2733 | 0.66 | 0.57 | 0.00 | 1.80 | 0.08 | 2.12 | 1.03 | 0.78 | 1.03 | 0.00 | 1.69 | 0.00 |
| S2H6N4 | 2383 | 1.13 | 1.04 | 0.00 | 0.00 | 0.47 | 0.00 | 0.00 | 0.14 | 0.00 | 1.76 | 0.00 | 0.00 |
| S1H7N6F2 | 2953 | 0.77 | 0.00 | 0.00 | 0.83 | 0.00 | 0.00 | 0.00 | 0.00 | 0.00 | 1.11 | 0.00 | 0.00 |
| S1H8N7F1 | 3172 | 0.00 | 0.00 | 0.00 | 1.66 | 0.00 | 0.00 | 0.00 | 0.00 | 0.00 | 0.74 | 0.00 | 0.00 |
| S1H4N4F1 | 1914 | 1.26 | 2.30 | 1.94 | 0.00 | 2.00 | 1.87 | 0.99 | 2.32 | 2.38 | 0.00 | 1.61 | 1.06 |
| S3H6N5 | 2878 | 0.00 | 0.00 | 0.00 | 0.00 | 0.00 | 1.33 | 1.92 | 0.42 | 0.00 | 0.00 | 0.37 | 0.00 |
| S1H6N4F1Ac | 2280 | 0.72 | 1.86 | 2.86 | 0.00 | 3.05 | 5.74 | 0.00 | 0.72 | 1.93 | 0.72 | 2.23 | 3.35 |
| S2H6N5F2 | 2879 | 0.00 | 0.00 | 0.00 | 0.00 | 0.48 | 0.00 | 0.00 | 0.47 | 0.00 | 1.11 | 0.53 | 0.00 |
| S1H5N5 | 2133 | 0.00 | 0.84 | 1.81 | 0.00 | 1.22 | 2.68 | 0.00 | 0.44 | 1.78 | 0.81 | 1.24 | 0.73 |
| S2H5N5F1 | 2570 | 0.00 | 0.79 | 1.74 | 0.00 | 0.76 | 0.00 | 0.00 | 0.12 | 0.49 | 0.72 | 1.55 | 2.04 |
| S2H7N6F1 | 3098 | 0.00 | 0.00 | 0.00 | 0.00 | 0.00 | 0.00 | 0.67 | 0.04 | 0.00 | 0.00 | 0.09 | 1.66 |
| S1H6N6F1 | 2644 | 0.00 | 0.64 | 1.92 | 0.00 | 0.88 | 2.27 | 0.00 | 1.21 | 2.37 | 0.00 | 1.29 | 3.00 |
| S1H5N6F2 | 2482 | 0.00 | 1.20 | 1.86 | 0.00 | 0.00 | 1.92 | 0.00 | 0.57 | 1.54 | 0.00 | 0.54 | 1.20 |
| S1H7N5F1Ac | 2645 | 0.00 | 0.00 | 0.98 | 0.00 | 0.56 | 2.02 | 0.00 | 0.55 | 0.56 | 0.00 | 0.92 | 2.12 |
| S1H5N5F3 | 2571 | 0.00 | 0.23 | 0.00 | 0.00 | 0.23 | 0.00 | 0.00 | 0.68 | 1.50 | 0.00 | 0.91 | 1.26 |
| S1H4N4 | 1768 | 0.00 | 0.55 | 1.17 | 0.00 | 0.46 | 0.00 | 0.00 | 0.17 | 0.00 | 0.00 | 0.32 | 0.00 |
| S2H2N3F1 | 1678 | 1.04 | 2.17 | 3.95 | 0.00 | 1.87 | 4.08 | 0.94 | 2.12 | 2.86 | 0.89 | 2.58 | 1.69 |
| S2H4N3F1 | 2002 | 0.00 | 1.26 | 2.86 | 0.00 | 1.03 | 2.35 | 1.27 | 1.62 | 0.95 | 0.00 | 1.58 | 0.99 |
| S2H3N3F1 | 1840 | 0.00 | 0.78 | 1.42 | 0.00 | 0.58 | 1.92 | 1.01 | 0.55 | 0.00 | 0.00 | 0.51 | 0.97 |
| S2H4N2F1 | 1799 | 0.00 | 0.00 | 1.22 | 0.00 | 0.43 | 1.92 | 0.00 | 0.07 | 0.60 | 0.00 | 0.00 | 0.89 |

TABLE 11
Relative proportions (%) of neutral N-glycan signals in hESC and differentiated cell lines.

| Proposed | | | | | | | | | | | | | |
|---|---|---|---|---|---|---|---|---|---|---|---|---|---|
| composition | m/z | FES 21 | EB 21 | St.3 21 | FES 22 | EB 22 | St.3 22 | FES 29 | EB 29 | St.3 29 | FES 30 | EB 30 | St.3 30 |
| H9N2 | 1905 | 19.19 | 14.65 | 17.06 | 18.69 | 15.98 | 15.26 | 19.92 | 1.07 | 0.00 | 18.96 | 0.00 | 0.00 |
| H8N2 | 1743 | 21.08 | 14.38 | 16.76 | 14.51 | 15.32 | 16.45 | 20.67 | 0.87 | 0.87 | 21.12 | 1.56 | 1.04 |
| H6N2 | 1419 | 18.41 | 18.31 | 14.47 | 16.18 | 17.95 | 16.33 | 16.74 | 1.66 | 2.13 | 16.35 | 2.51 | 1.22 |
| H7N2 | 1581 | 13.01 | 11.25 | 10.79 | 10.10 | 10.86 | 11.15 | 12.27 | 1.76 | 1.62 | 12.17 | 2.44 | 1.47 |
| H5N2 | 1257 | 9.75 | 14.50 | 11.50 | 10.71 | 14.37 | 11.51 | 8.13 | 3.10 | 3.87 | 8.27 | 3.78 | 2.33 |
| H3N2F1 | 1079 | 1.19 | 3.78 | 4.20 | 3.37 | 2.97 | 4.64 | 0.95 | 2.62 | 2.39 | 1.12 | 3.01 | 2.31 |
| H4N2 | 1095 | 2.07 | 2.87 | 2.80 | 2.56 | 2.84 | 2.36 | 1.63 | 0.35 | 0.43 | 1.43 | 0.78 | 0.78 |
| H10N2 | 2067 | 2.82 | 1.81 | 1.87 | 2.79 | 2.05 | 1.76 | 2.25 | 0.38 | 0.33 | 2.14 | 0.43 | 2.29 |
| N2N2F1 | 917 | 0.56 | 2.34 | 2.82 | 1.23 | 1.67 | 3.62 | 0.35 | 0.43 | 0.43 | 0.47 | 0.60 | 0.24 |
| H3N2 | 933 | 1.10 | 2.20 | 2.30 | 2.08 | 1.82 | 2.12 | 0.74 | 13.30 | 12.32 | 0.61 | 11.22 | 8.25 |
| H2N2 | 771 | 0.43 | 1.07 | 1.97 | 0.77 | 0.73 | 1.96 | 0.00 | 0.65 | 1.04 | 0.00 | 0.81 | 1.11 |
| H1N2 | 609 | 0.00 | 0.00 | 0.00 | 0.56 | 0.00 | 0.00 | 2.90 | 0.65 | 0.42 | 3.99 | 0.53 | 0.36 |
| H5N2F1 | 1403 | 0.32 | 0.44 | 0.41 | 0.27 | 0.40 | 0.57 | 0.00 | 0.00 | 0.22 | 0.00 | 0.31 | 0.35 |
| H4N2F1 | 1241 | 0.26 | 0.46 | 0.42 | 0.36 | 0.46 | 0.35 | 0.21 | 0.07 | 0.30 | 0.14 | 0.30 | 0.30 |
| H6N2F1 | 1565 | 0.00 | 0.14 | 0.17 | 0.00 | 0.21 | 0.42 | 0.00 | 0.53 | 0.55 | 0.00 | 0.56 | 0.34 |
| H11N2 | 2229 | 0.00 | 0.10 | 0.12 | 0.24 | 0.00 | 0.00 | 0.10 | 16.44 | 16.44 | 0.07 | 17.49 | 12.47 |
| H6N3 | 1622 | 0.57 | 0.86 | 0.97 | 1.51 | 0.96 | 0.91 | 0.58 | 0.64 | 0.56 | 0.53 | 0.84 | 0.69 |
| H5N3 | 1460 | 0.50 | 0.58 | 0.87 | 1.27 | 0.70 | 0.61 | 0.51 | 1.11 | 0.96 | 0.55 | 0.72 | 0.84 |
| H3N3F1 | 1282 | 0.33 | 0.48 | 0.78 | 0.59 | 0.48 | 0.54 | 0.35 | 0.85 | 1.06 | 0.41 | 0.40 | 0.68 |
| H4N3F1 | 1444 | 0.55 | 0.46 | 0.44 | 0.77 | 0.66 | 0.49 | 0.73 | 0.08 | 0.22 | 0.65 | 0.28 | 0.33 |
| H3N3 | 1136 | 0.28 | 0.28 | 0.78 | 0.64 | 0.43 | 0.39 | 0.31 | 0.08 | 0.27 | 0.33 | 0.05 | 0.03 |
| H4N3 | 1298 | 0.59 | 0.45 | 0.74 | 0.80 | 0.63 | 0.52 | 0.45 | 0.22 | 0.23 | 0.50 | 0.13 | 0.06 |
| H5N3F1 | 1606 | 0.28 | 0.34 | 0.30 | 0.74 | 0.32 | 0.20 | 0.23 | 10.77 | 10.69 | 0.11 | 11.14 | 9.82 |
| H2N3F1 | 1120 | 0.00 | 0.35 | 0.66 | 0.00 | 0.33 | 0.41 | 0.00 | 0.06 | 0.00 | 0.00 | 0.08 | 0.11 |
| H6N3F1 | 1768 | 0.33 | 0.32 | 0.14 | 0.39 | 0.21 | 0.29 | 0.00 | 0.61 | 0.68 | 0.00 | 0.08 | 0.25 |
| H4N3F2 | 1590 | 0.00 | 0.17 | 0.15 | 0.00 | 0.24 | 0.00 | 0.00 | 1.76 | 1.17 | 0.17 | 0.97 | 1.23 |
| H5N4 | 1663 | 2.29 | 1.89 | 1.14 | 1.78 | 1.82 | 0.91 | 2.19 | 0.63 | 0.52 | 2.75 | 0.12 | 0.28 |
| H5N4F1 | 1809 | 1.33 | 1.27 | 0.57 | 1.50 | 1.37 | 0.66 | 3.86 | 1.91 | 2.07 | 3.69 | 1.30 | 2.68 |
| H3N4F1 | 1485 | 0.41 | 0.47 | 0.67 | 1.03 | 0.64 | 0.77 | 0.57 | 0.31 | 0.46 | 0.55 | 0.06 | 0.17 |
| H5N5 | 1866 | 0.00 | 0.11 | 0.43 | 1.33 | 0.32 | 0.55 | 0.00 | 0.81 | 0.82 | 0.00 | 0.06 | 0.28 |
| H4N4F1 | 1647 | 0.32 | 0.40 | 0.34 | 0.52 | 0.40 | 0.40 | 0.46 | 14.86 | 15.30 | 0.38 | 14.82 | 17.75 |
| H5N4F2 | 1955 | 0.42 | 0.26 | 0.18 | 0.00 | 0.38 | 0.31 | 0.83 | 0.23 | 0.16 | 0.89 | 0.04 | 0.40 |
| H4N5 | 1704 | 0.00 | 0.00 | 0.27 | 1.35 | 0.07 | 0.33 | 0.00 | 0.09 | 0.00 | 0.00 | 0.33 | 0.38 |
| H6N5F1 | 2174 | 0.36 | 0.27 | 0.11 | 0.21 | 0.22 | 0.00 | 0.73 | 2.07 | 1.13 | 0.50 | 1.03 | 1.09 |
| H5N4F3 | 2101 | 0.21 | 0.22 | 0.14 | 0.00 | 0.27 | 0.21 | 0.47 | 0.11 | 0.34 | 0.47 | 0.02 | 0.29 |
| H4N5F1 | 1850 | 0.00 | 0.20 | 0.21 | 0.28 | 0.25 | 0.32 | 0.00 | 0.48 | 0.41 | 0.00 | 0.07 | 0.36 |
| H6N5 | 2028 | 0.34 | 0.19 | 0.12 | 0.27 | 0.25 | 0.00 | 0.56 | 0.89 | 1.01 | 0.30 | 0.19 | 0.60 |
| H3N5F1 | 1688 | 0.00 | 0.21 | 0.29 | 0.00 | 0.19 | 0.35 | 0.18 | 14.28 | 15.44 | 0.14 | 16.85 | 22.44 |
| H4N4 | 1501 | 0.02 | 0.27 | 0.40 | 0.18 | 0.08 | 0.36 | 0.00 | 0.30 | 0.00 | 0.00 | 0.10 | 0.36 |
| H4N5F2 | 1996 | 0.00 | 0.23 | 0.14 | 0.00 | 0.23 | 0.31 | 0.15 | 0.06 | 0.00 | 0.00 | 0.20 | 0.40 |
| H3N4 | 1339 | 0.00 | 0.34 | 0.52 | 0.00 | 0.00 | 0.23 | 0.00 | 0.22 | 0.25 | 0.00 | 0.27 | 0.33 |
| H4N4F2 | 1793 | 0.00 | 0.22 | 0.16 | 0.00 | 0.23 | 0.30 | 0.00 | 0.19 | 0.12 | 0.14 | 0.04 | 0.10 |
| H6N4 | 1825 | 0.00 | 0.07 | 0.32 | 0.10 | 0.00 | 0.37 | 0.00 | 0.16 | 0.10 | 0.00 | 0.04 | 0.10 |
| H4N5F3 | 2142 | 0.50 | 0.11 | 0.06 | 0.00 | 0.00 | 0.00 | 0.00 | 1.65 | 2.00 | 0.10 | 2.22 | 2.25 |
| H5N6F2 | 2361 | 0.00 | 0.14 | 0.00 | 0.00 | 0.12 | 0.00 | 0.00 | 0.21 | 0.00 | 0.00 | 0.31 | 0.13 |
| H5N5F3 | 2304 | 0.00 | 0.15 | 0.16 | 0.00 | 0.17 | 0.31 | 0.00 | 0.11 | 0.00 | 0.00 | 0.43 | 0.03 |
| H5N5F1 | 2012 | 0.00 | 0.12 | 0.12 | 0.27 | 0.12 | 0.00 | 0.00 | 0.19 | 0.14 | 0.00 | 0.06 | 0.09 |
| H7N4 | 1987 | 0.00 | 0.07 | 0.11 | 0.00 | 0.00 | 0.00 | 0.00 | 0.09 | 0.13 | 0.00 | 0.03 | 0.09 |
| H3N5 | 1542 | 0.00 | 0.21 | 0.00 | 0.05 | 0.00 | 0.13 | 0.00 | 0.09 | 0.17 | 0.00 | 0.04 | 0.10 |
| H2N4F1 | 1323 | 0.19 | 0.00 | 0.08 | 0.00 | 0.30 | 0.33 | 0.00 | 0.00 | 0.21 | 0.00 | 0.38 | 0.42 |

TABLE 12
Proposed structures for acidic N-glycan signals in hESC or differentiated cells, symbols Table 13.

| m/z | structure |
|---|---|
| 1151 | 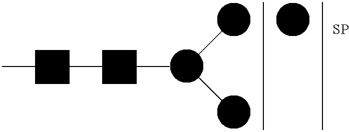 |
| 1338 | 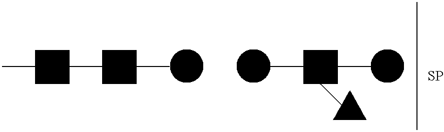 |
| 1354 | 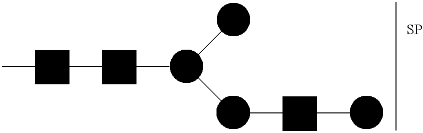 |
| 1362 | |
| 1403 | 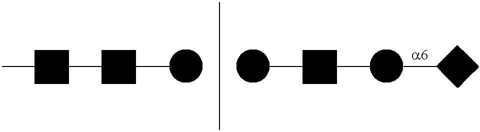 |
| 1475 | |
| 1500 | 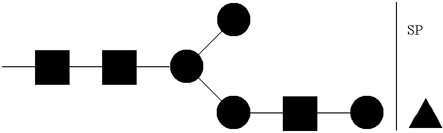 |
| 1516 | |
| 1541 | 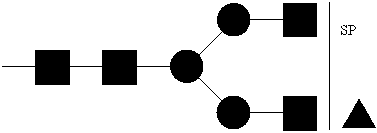 |
| 1549 | 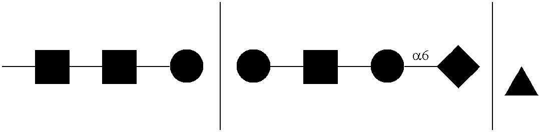 |
| 1557 | 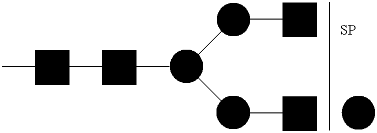 |
| 1565 | 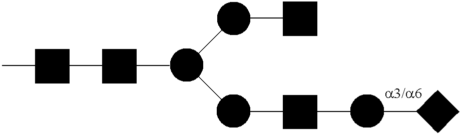 |
| 1637 | |
| 1678 | 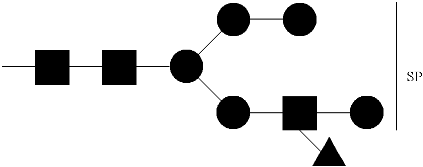 |
| 1703 | 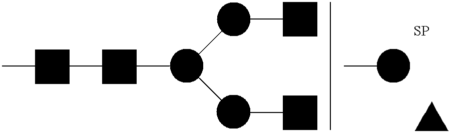 |
| 1711 | 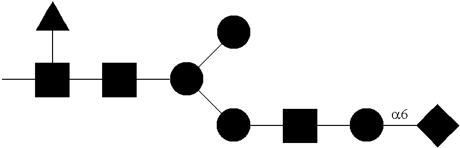 |
| 1719 | 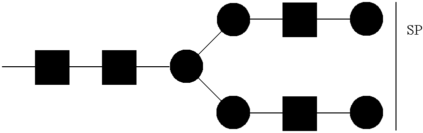 |
| 1727 | 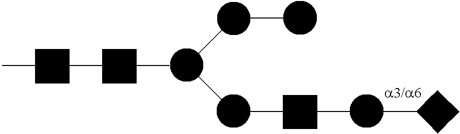 |
| 1744 | |
| 1752 | |
| 1760 | |
| 1768 | 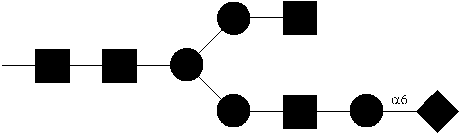 |
| 1791 | 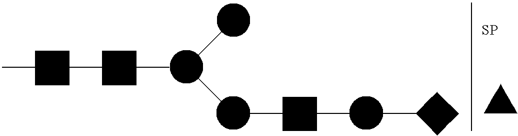 |
| 1799 | |
| 1808 | 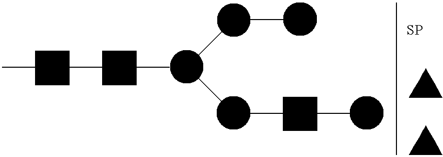 |
| 1824 | 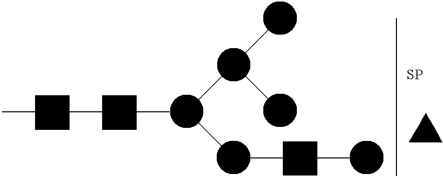 |
| 1831 | |
| 1840 | |
| 1849 | 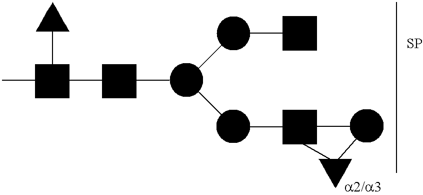 |
| 1865 | 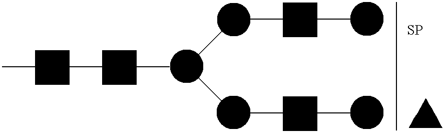 |
| 1873 | 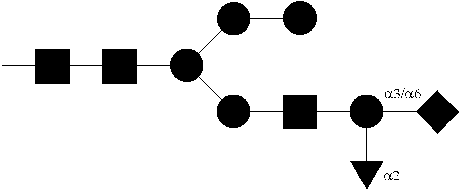 |
| 1889 | 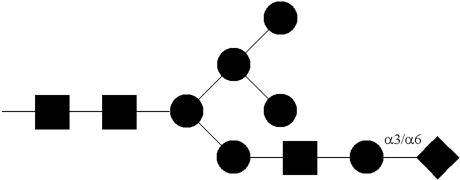 |
| 1906 | |
| 1914 | 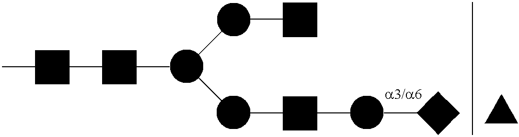 |
| 1930 | 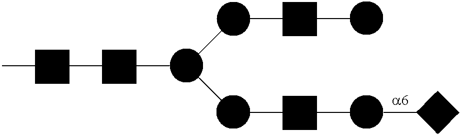 |
| 1946 | 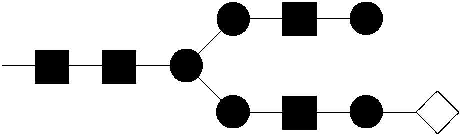 |
| 1947 | |
| 1971 | |
| 2002 | |
| 2003 | |
| 2010 | 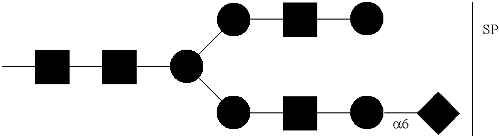 |
| 2011 | 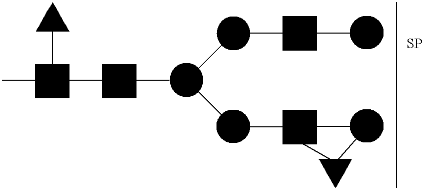 |
| 2018 | |
| 2027 | 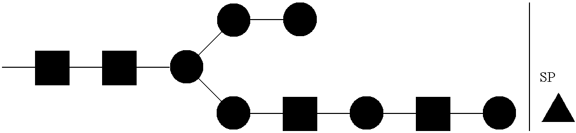 |
| 2035 | 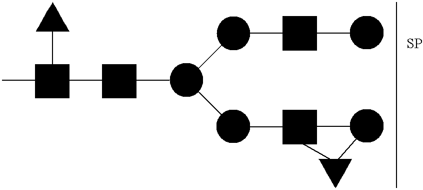 |
| 2051 | 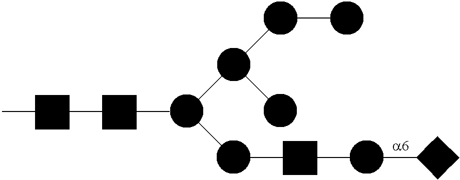 |
| 2052 | 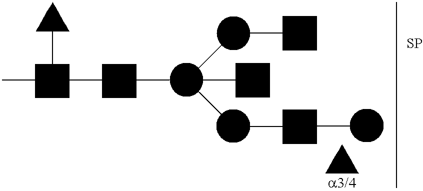 |
| 2068 | 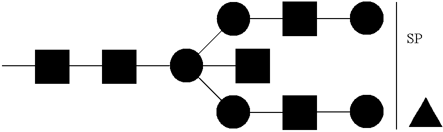 |
| 2076 | 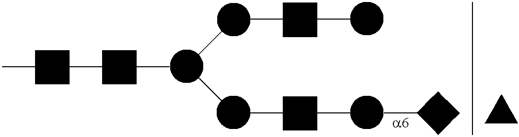 |
| 2082 | |
| 2092 | 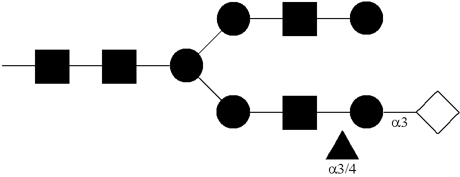 |
| 2117 | 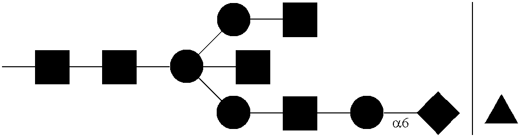 |
| 2133 | 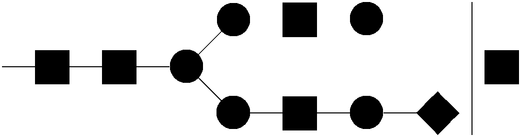 |
| 2156 | 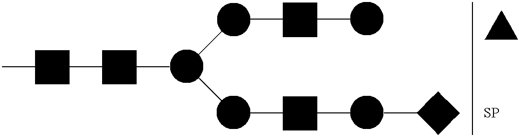 |
| 2157 | 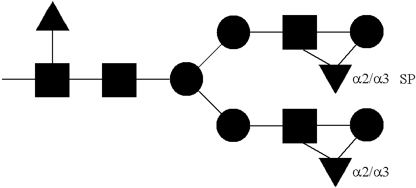 |
| 2164 | |
| 2174 | |
| 2178 | |
| 2214 | |
| 2221 | 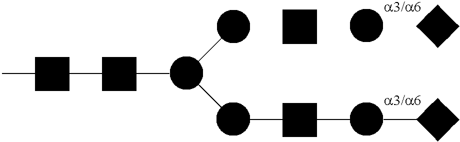 |
| 2222 | 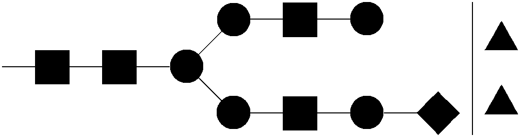 |
| 2230 | |
| 2237 | 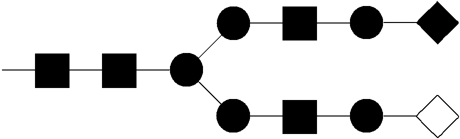 |
| 2238 | |
| 2239 | |
| 2246 | |
| 2253 | 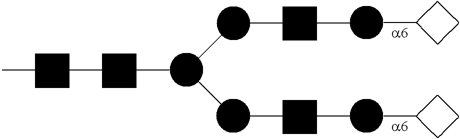 |
| 2254 | |
| 2263 | 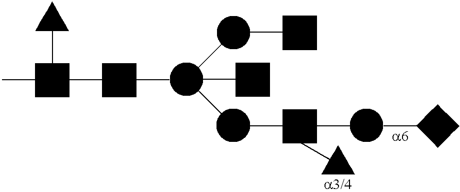 |
| 2279 | 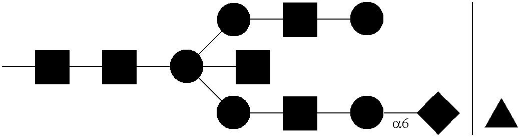 |
| 2280 | |
| 2295 | 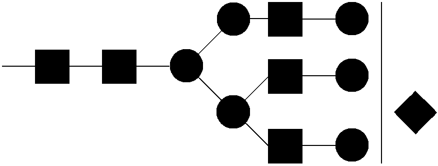 |
| 2302 | 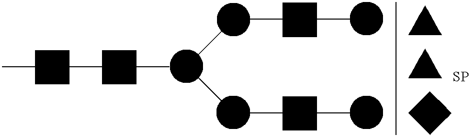 |
| 2319 | 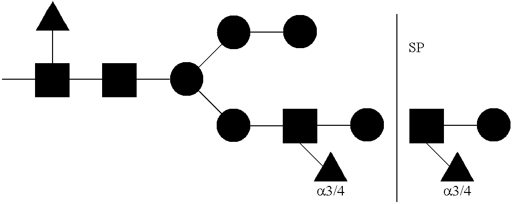 |
| 2320 | |
| 2321 | |
| 2367 | 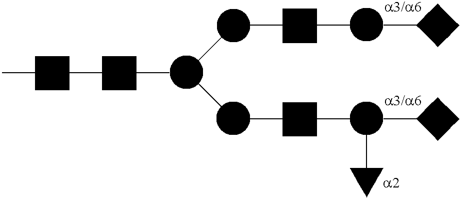 |
| 2368 | 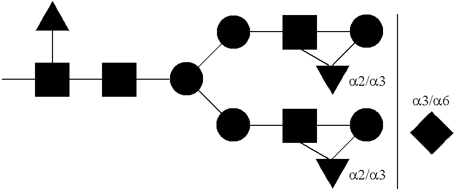 |
| 2376 | 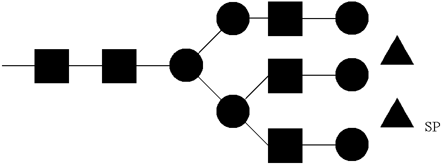 |
| 2383 | |
| 2384 | 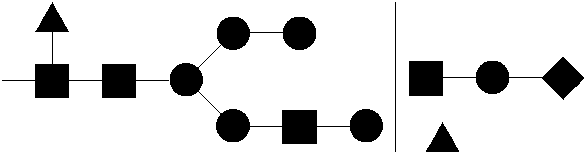 |
| 2390 | |
| 2391 | |
| 2400 | 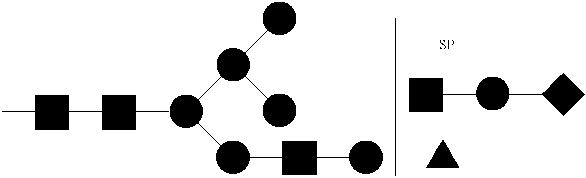 |
| 2408 | 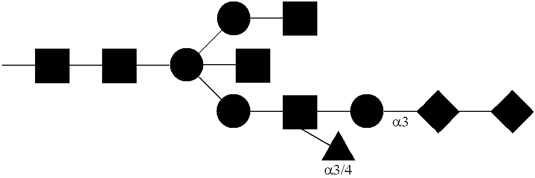 |
| 2425 | 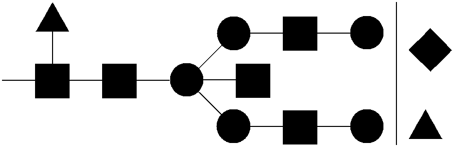 |
| 2433 | 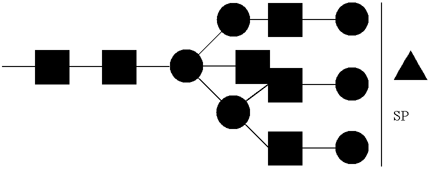 |
| 2441 | 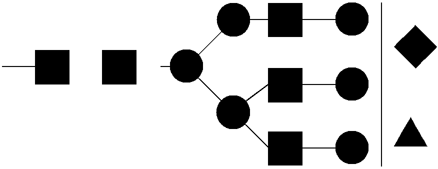 |
| 2447 | 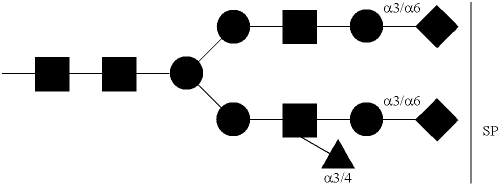 |
| 2448 | 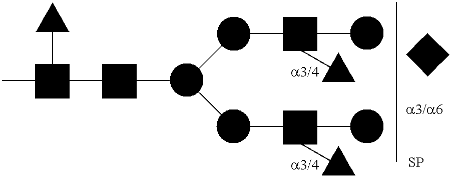 |
| 2456 | |
| 2457 | 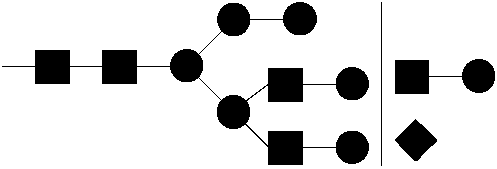 |
| 2482 | 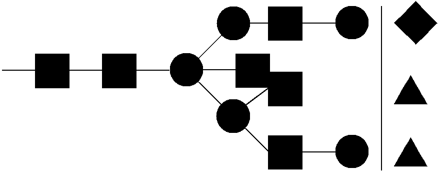 |
| 2483 | 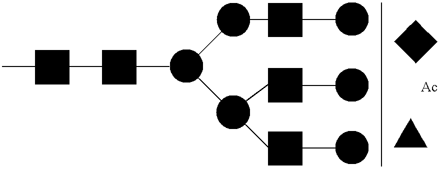 |
| 2512 | |
| 2521 | 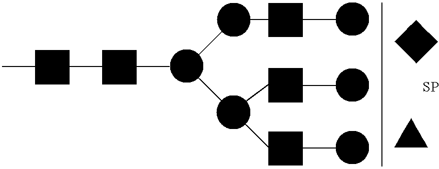 |
| 2522 | 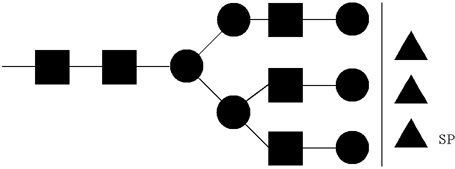 |
| 2528 | 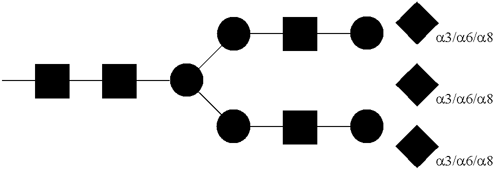 |
| 2529 | 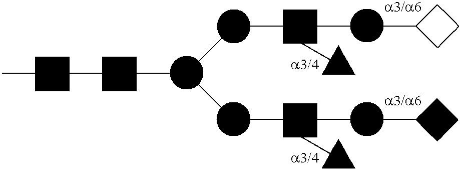 |
| 2544 | 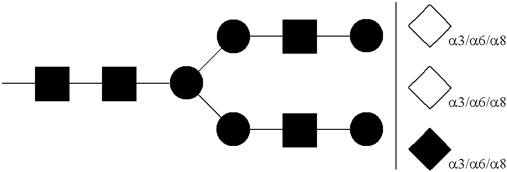 |
| 2570 | 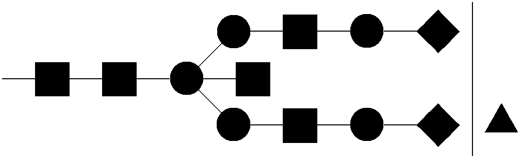 |
| 2571 | 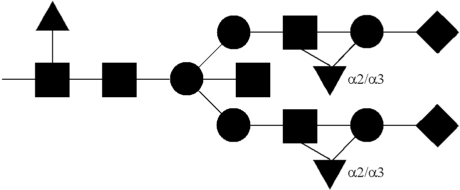 |
| 2579 | 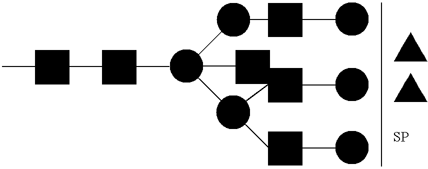 |
| 2586 | 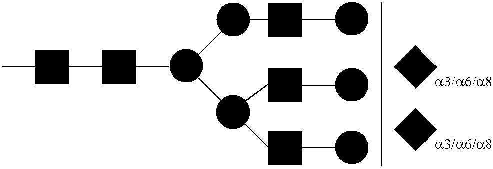 |
| 2587 | 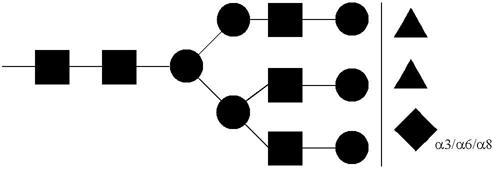 |
| 2603 | 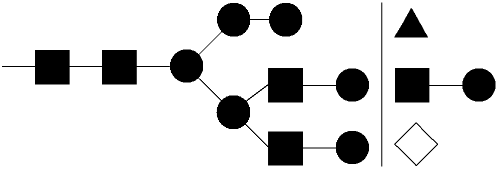 |
| 2627 | 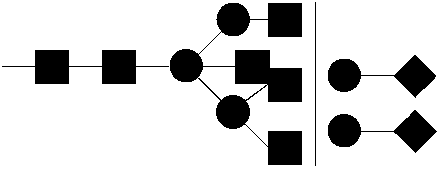 |
| 2644 | 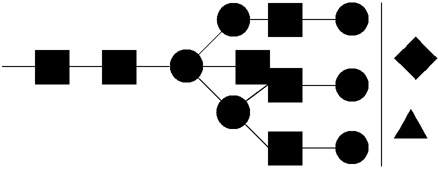 |
| 2645 | 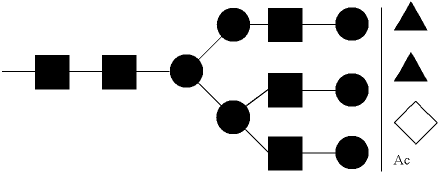 |
| 2660 | 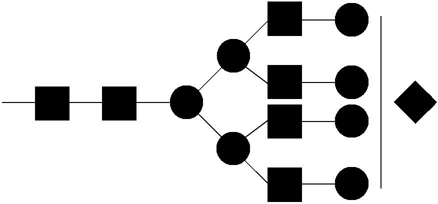 |
| 2668 | 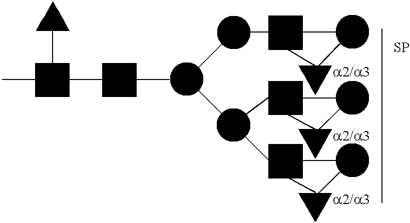 |
| 2683 | 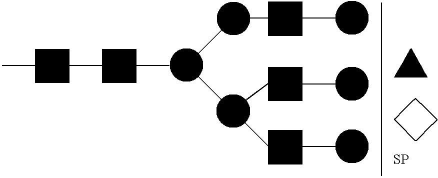 |
| 2714 | 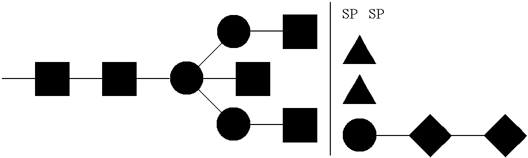 |
| 2725 | 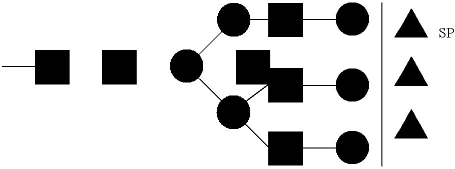 |
| 2732 | 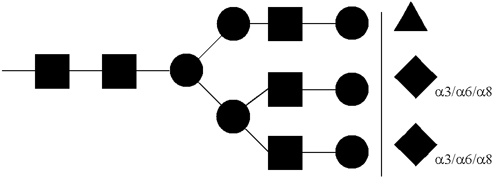 |
| 2733 | 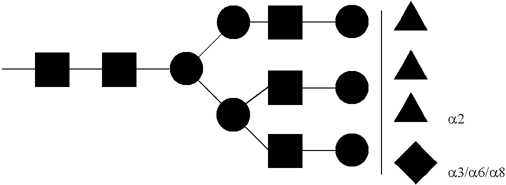 |
| 2791 | 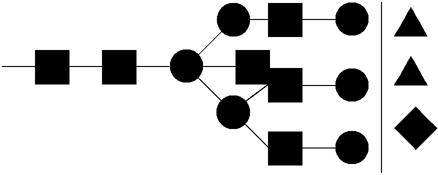 |
| 2806 | |
| 2807 | 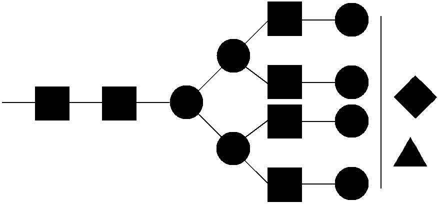 |
| 2813 | 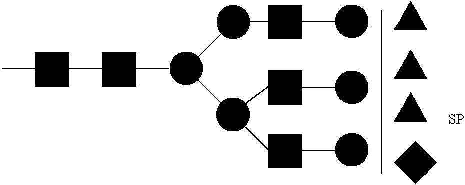 |
| 2848 | 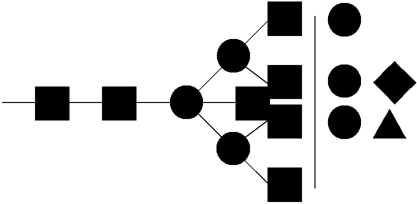 |
| 2864 | 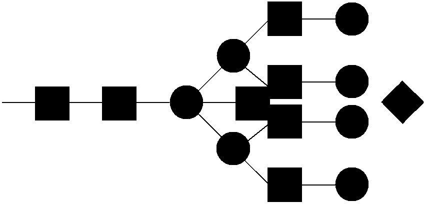 |
| 2878 | 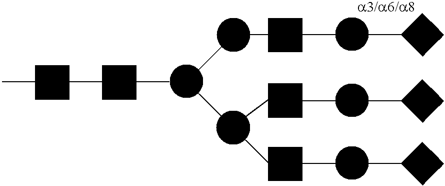 |
| 2879 | 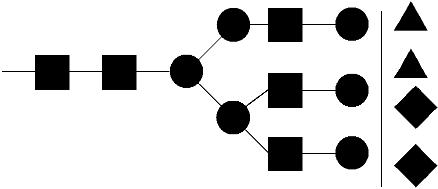 |
| 2880 | 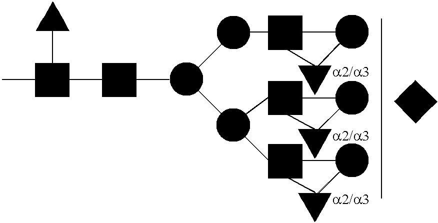 |
| 2886 | 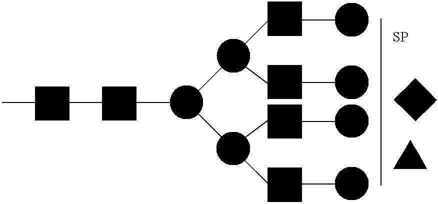 |
| 2887 | 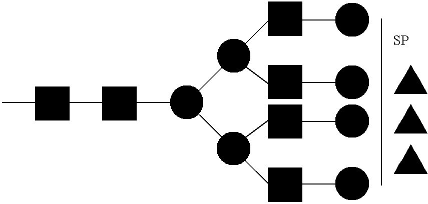 |
| 2936 | 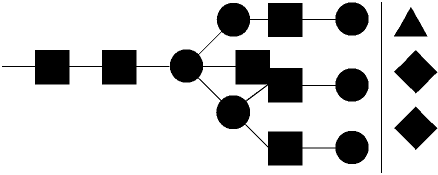 |
| 2953 | 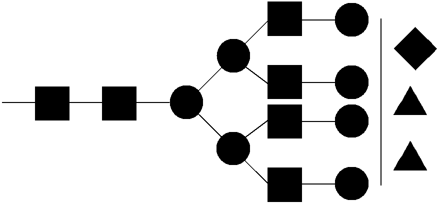 |
| 3024 | 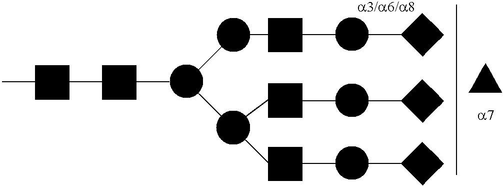 |
| 3025 | 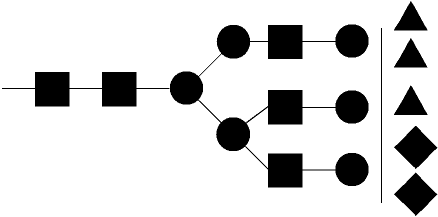 |
| 3026 | 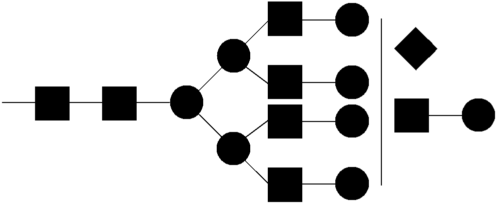 |
| 3098 | 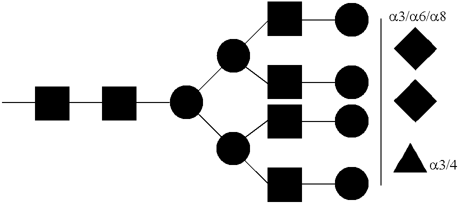 |
| 3099 | |
| 3170 | 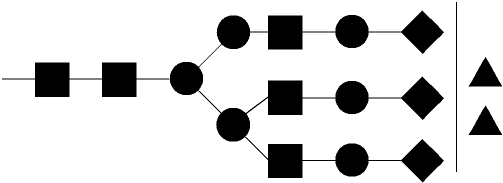 |
| 3172 | 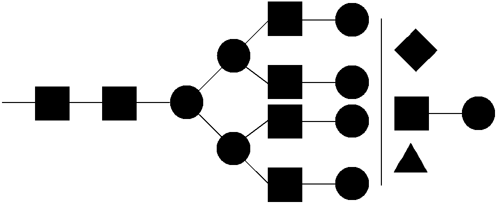 |
| 3245 | 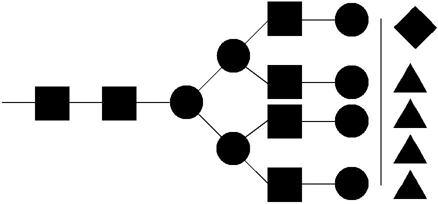 |
| 3317 | 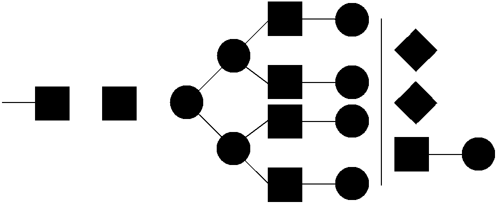 |
| 3390 | 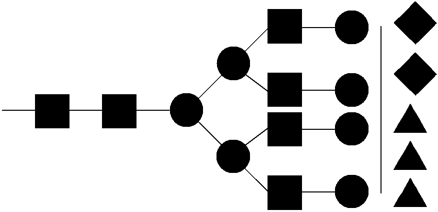 |
| 3463 | 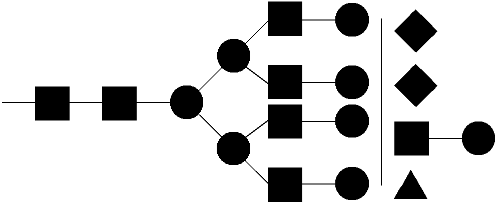 |
| 3608 | 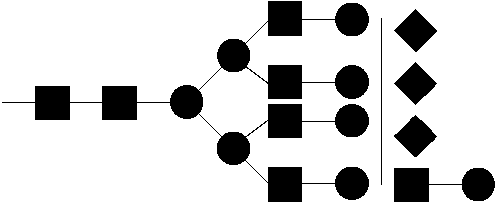 |
| 3610 | 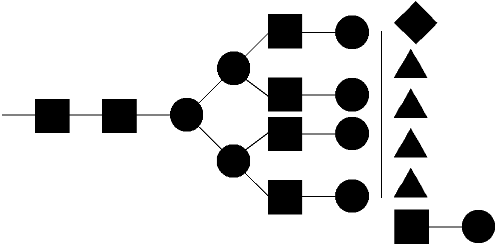 |
| 3682 | 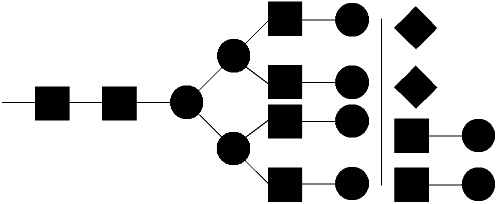 |
| 3756 | 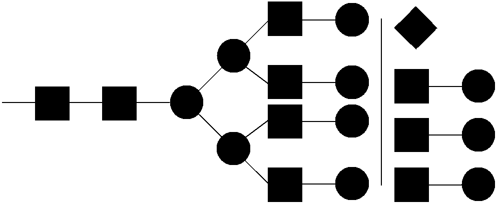 |

TABLE 13
Proposed structures for neutral N-glycan signals detected
in hESC or differentiated cells. Symbols Table 14.

| m/z | Structure |
|---|---|
| 568,19 | 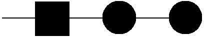 |
| 609,21 | 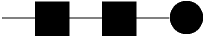 |
| 714,24 | |
| 730,24 | 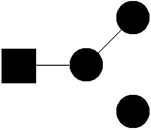 |
| 755,27 | 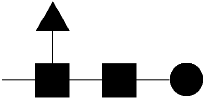 |
| 771,26 | 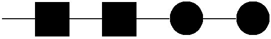 |
| 892,29 | 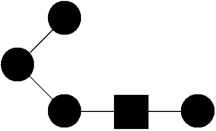 |
| 901,33 | |
| 917,32 | 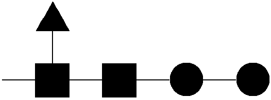 |
| 933,31 | 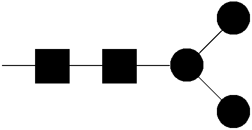 |
| 1031,33 | |
| 1054,34 | 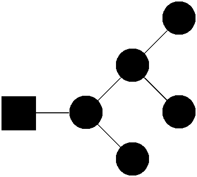 |
| 1079,38 | 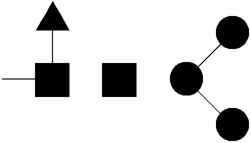 |
| 1095,37 | 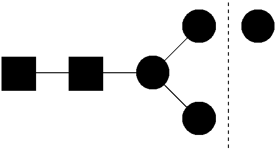 |
| 1120,4 | 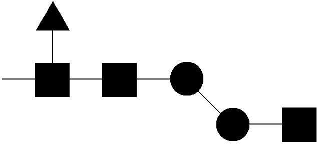 |
| 1136,4 | 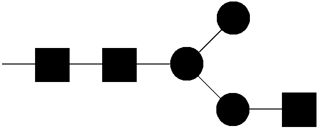 |
| 1209,44 | |
| 1216,4 | 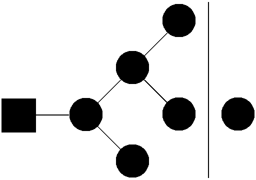 |
| 1225,43 | |
| 1241,43 | 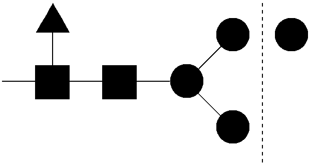 |
| 1257,42 | 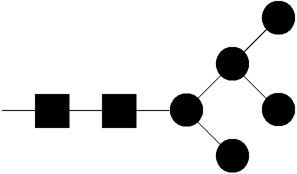 |
| 1266,46 | |
| 1282,45 | 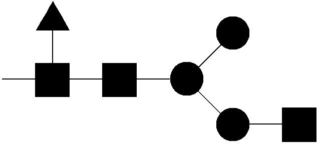 |
| 1298,45 | 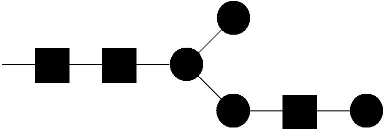 |
| 1323,48 | 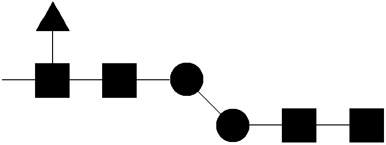 |
| 1339,48 | 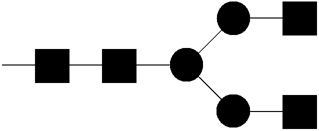 |
| 1378,45 | 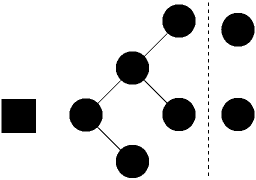 |
| 1393 | |
| 1403,48 | 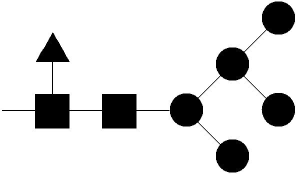 |
| 1419,48 | 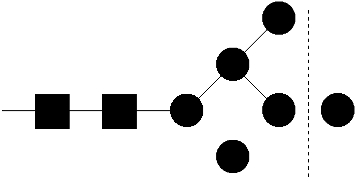 |
| 1444,51 | 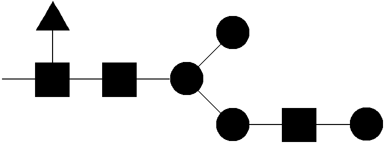 |
| 1460,5 | 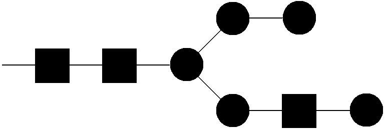 |
| 1485,53 | 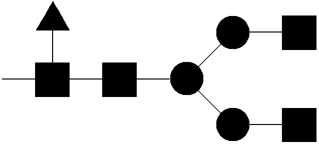 |
| 1501,53 | 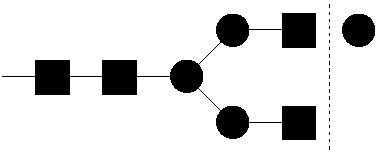 |
| 1517,55 | |
| 1540,5 | 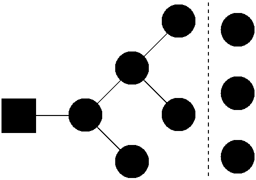 |
| 1542,56 | 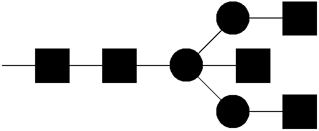 |
| 1555 | |
| 1565,53 | 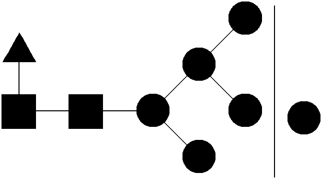 |
| 1581,53 | 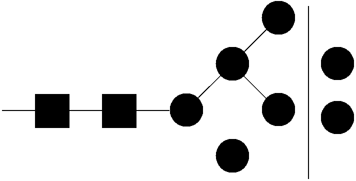 |
| 1590,57 | 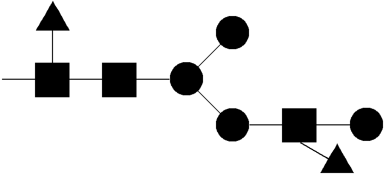 |
| 1606,56 | 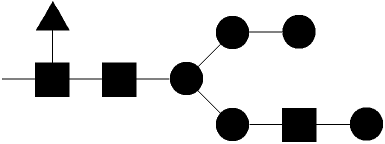 |
| 1622,56 | 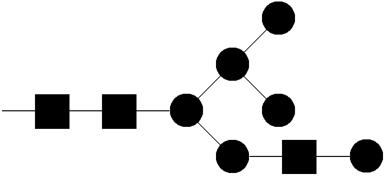 |
| 1631,59 | 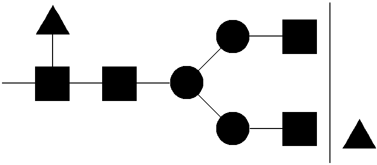 |
| 1647,59 | 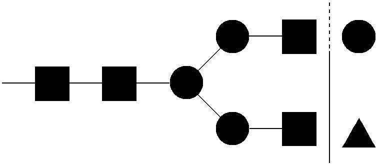 |
| 1663,58 | 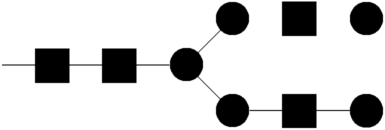 |
| 1688,61 | 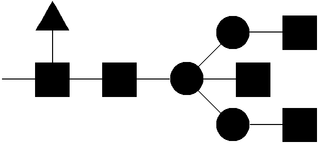 |
| 1702,56 | 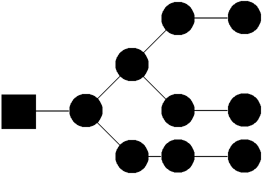 |
| 1704,61 | 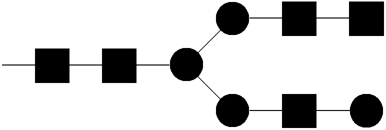 |
| 1717 | |
| 1720,63 | |
| 1743,58 | 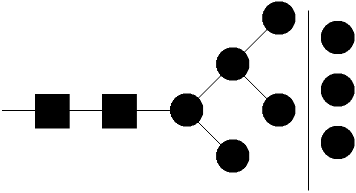 |
| 1752,62 | 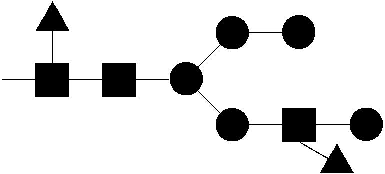 |
| 1768,61 | 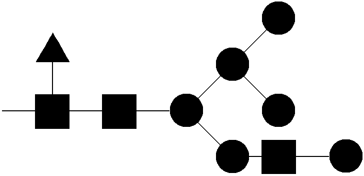 |
| 1784,61 | |
| 1793,64 | 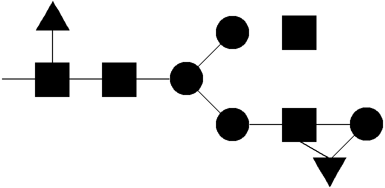 |
| 1809,64 | 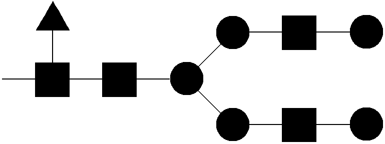 |
| 1825,63 | 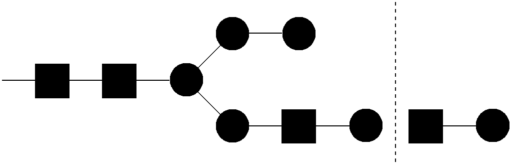 |
| 1850,67 | 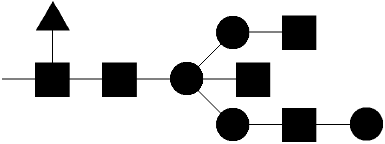 |
| 1864,61 | 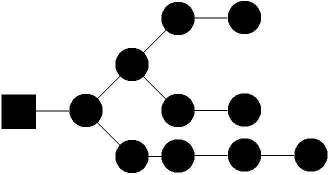 |
| 1866,66 | 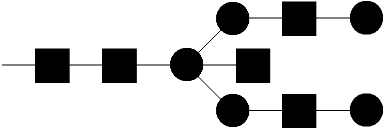 |
| 1882,68 | |
| 1905,63 | 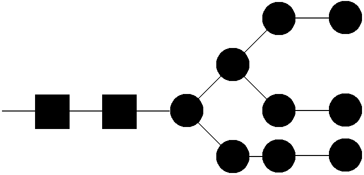 |
| 1914,67 | 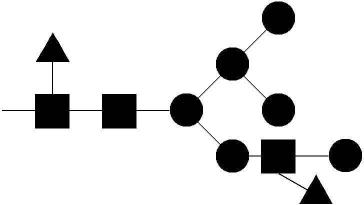 |
| 1955,7 | 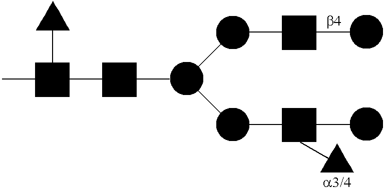 |
| 1971,69 | 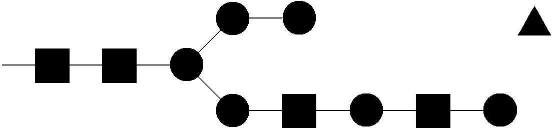 |
| 1980,73 | 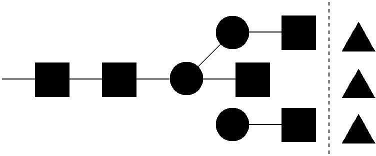 |
| 1987,69 | 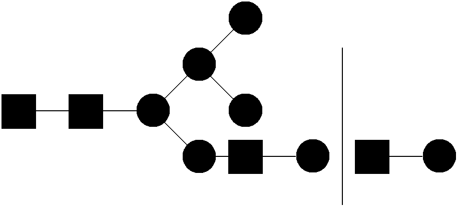 |
| 1996,72 | 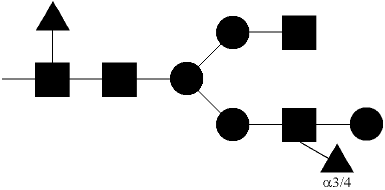 |
| 2012,72 | 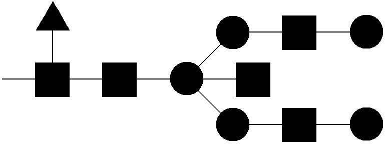 |
| 2019,7 | |
| 2021,76 | |
| 2028,71 | 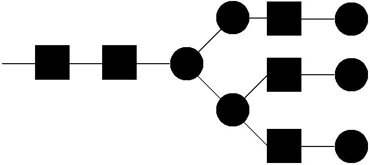 |
| 2037,75 | |
| 2041 | |
| 2053,75 | |
| 2067,69 | 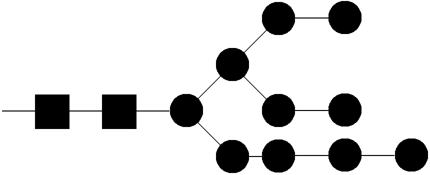 |
| 2101,76 | 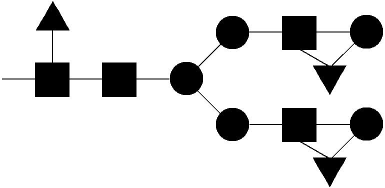 |
| 2117,75 | 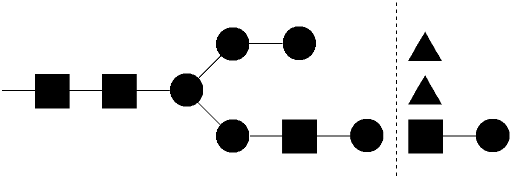 |
| 2126,79 | |
| 2133,75 | 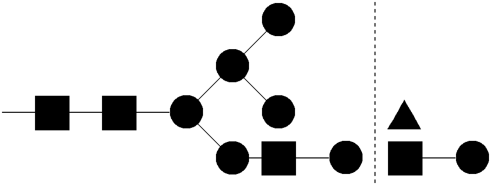 |
| 2142,78 | |
| 2149,74 | 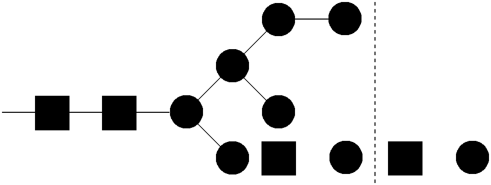 |
| 2158,78 | |
| 2174,77 | 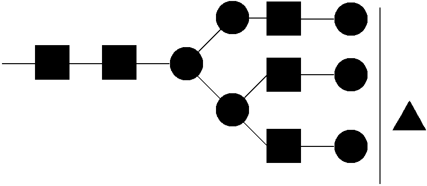 |
| 2183,81 | |
| 2190,77 | 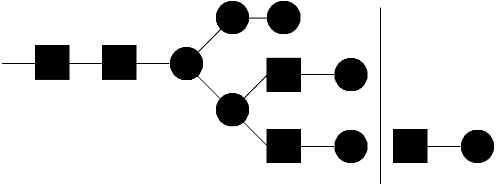 |
| 2199,8 | 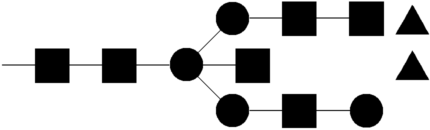 |
| 2215,8 | 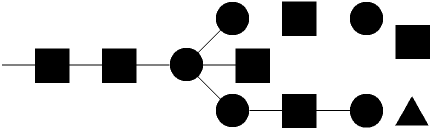 |
| 2229,74 | 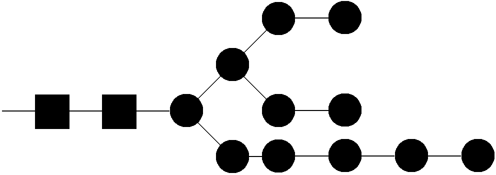 |
| 2231,79 | 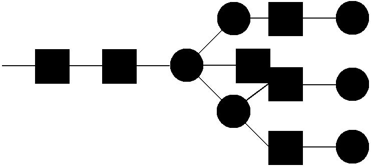 |
| 2304,84 | 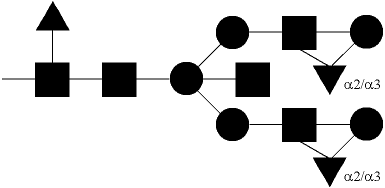 |
| 2320,83 | 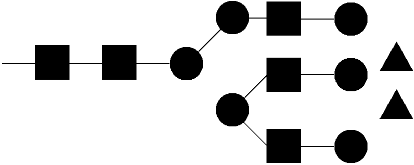 |
| 2361,87 | 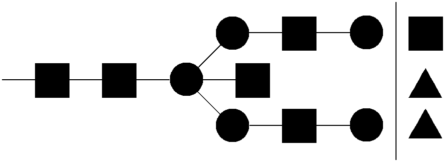 |
| 2391,79 | 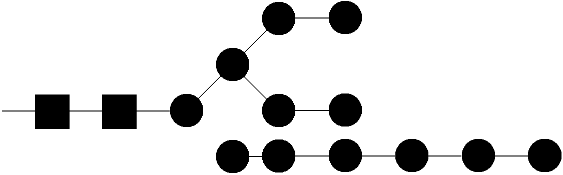 |
| 2393,85 | |
| 2466,89 | 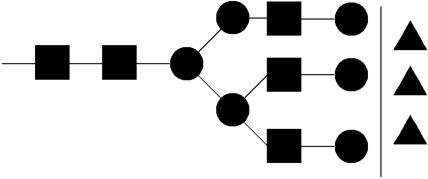 |

TABLE 14

| Lectin | epitope | | FES22 | FES30 | EB (29 +30 | MEF |
|---|---|---|---|---|---|---|
| PSA | Manα | 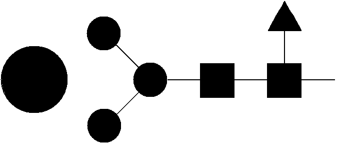 | − | − | | + |
| LTA | Lex | 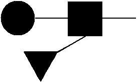 | + | − | − | + |
| UEA | H type 2 | 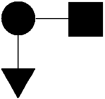 | + [symbol] | − [symbol] | 22+, 29− | +/− |
| MAA | Sα2-3 | 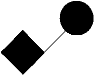 | + | + | + | − |
| SNA | Sα2-6 | 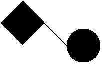 | (+/−) | (+/−) | | + |
| RCA | LN | 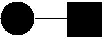 | + [symbol] | + | + | + |
| PNA | Galβ1- | 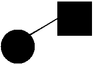 | + | + [symbol] | + | − |
| PWA | polyLN (I) | 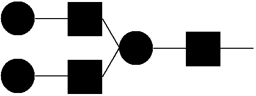 | + | + [symbol] | + | + |
| STA | polyLN (i) | 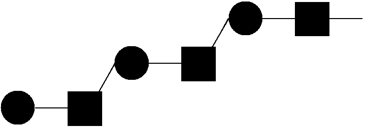 | (+/−) | − | | + |
| WFA | GalNAcβ | 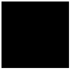 | + | + [symbol] | + | − |

Lectin staining of human embryonic stem cells. The glycan structures are presented in colour symbols, given at the end of Table 19. The reducing end of the N-glycans is on left for N-glycans in Tables 12 and 13, and on right in Tables 14-19 (mirror images to ones in 12 and 13). The linkages of N-glycans are indicated in NMR Tables 8 and 9, and in Tables 12-19 based on the Consortium for Functional Glycomics, USA recommendations, 1-4 linkages (Manβ4,GlcNAcβ4,Galα4 on Lactosylresidue in globostructres,GalNAcβ4 on on Lactosylresidue in ganliostructures) are horizontal −, 1-6 linkages (Manα6, NeuAc/sialic acidα6, NeuAc/sialic acidα6, GlcNAcβ6) are\in Tables 14-19, excepts Fucα6 above reducing end GlcNAc in, and/in Tables 12 and 13, 1-3 linkages (Manα3,Fucα3,Neu5Ac/Neu5Gc/sialic acidα3,Galβ3,GalNAcβ3,GalNAcα3GalNAcβ3 and GalNAcβ3 on Galα4 at non-reducing end of Forsman and Globoside(Gb4) and elongated globoseries glycolipid structures, respectively) are/in Tables 14-19, and\in Tables 12 and 13 (for N-glycan compatible structures, Fucα2 is indicated by vertical line below Galβ3/Galβ4-residue. SP in Tables 12 and 13 indicates sulphated or fosfate and is preferably sulfate on compelx type N-aglycans comprising N-acetyllactosamine residues and fosfate in High/Low Mannose glycans. In tables 14-19 S is sialic acid (preferably Neu5Ac and/or Neu5Gc), LN is N-cetyl-lactosamine, preferably Galβ4GlcNAc, LN type 1 is Galβ3GlcNAc, Lex is Lewis x, Ley is Lewis y, Leb is Lewis b. Regular abbreviations of plant leactins are used, these are available e.g. from catalog of EY Labs USA. MEF is mouse embryonic fibroblast feeder cell, FES indicates embryonic stem cell line and number specifies the line, EB is embryonic body.

TABLE 15
Antibody staining of human embryonic stem cells.
Antibodies are listed in Table 20.

| Epitope | | FES22,29,30 | MEF |
|---|---|---|---|
| globoH | 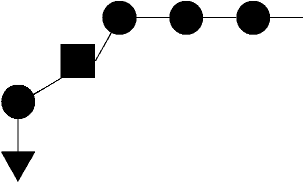 | −/+ [symbol] | − |
| H type 1 | 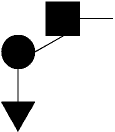 | + [symbol] | − |
| H type 2 | 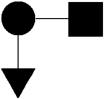 | + [symbol] | − |
| Leb, Ley, | 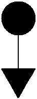 | − | + |
| Leb | 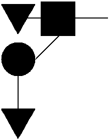 | −/+ | − |
| H type 2 | 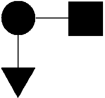 | −/+ | − |
| H type 2 | 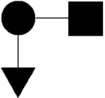 | −/+ | − |
| Ley | 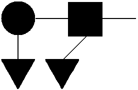 | ? | ? |
| LN (1) | 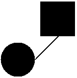 | + | − |

TABLE 16
Antibody staining of human embryonic stem cells.

| Epitope | | FES22,29,30 | MEF |
|---|---|---|---|
| Forssman | 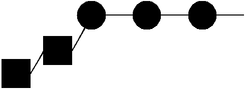 | − | −/+ |
| Low Man | 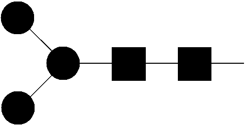 | −/+ | − |
| Globoside | 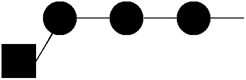 | −/+ | −/+ |
| LacdiNAc | 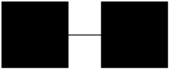 | −/+ | −/+ |
| GM3 | 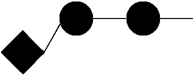 | + | + |
| GM3 | 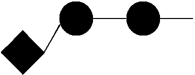 | + | + |
| Lex | 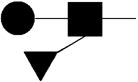 | −? | −? |
| sLex | 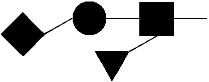 | −? | −? |
| sLea | 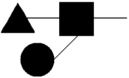 | − | − |

TABLE 17
FACS analysis (lectins) of human embryonic stem cells (% of positive cells).

| | | | FES29 | | FES30 | staining |
|---|---|---|---|---|---|---|
| Lectin | Epitope | | (MEF) | MEF | (matrigel) | (FES30) |
| PNA | Galβ1-3GalNAc | 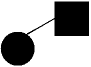 | 80% | 20% | 84% | + |
| PSA | Manα | 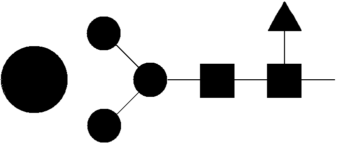 | 51% | 64% | 54% | − |
| MAA | Sα2-3 | 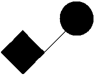 | 27% | 9% | 33% | + |
| PWA | polyLN (I) | 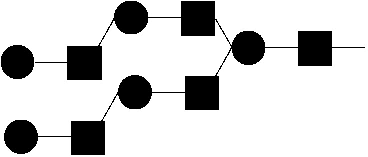 | 3% | 11% | 1% | + |
| UEA | H type 2 | 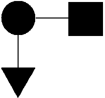 | 63% | 2% | 42% | − |
| STA | polyLN (i) | 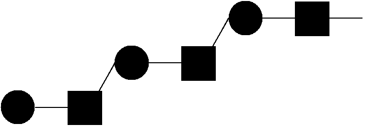 | 9% | | | − |
| MBL | Manα | 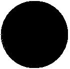 | 0% | | | |

TABLE 18
FACS analysis (antibodies) of human embryonic stem cells (% of positive cells).

| | | FES29 | | FES30 | Staining |
|---|---|---|---|---|---|
| epitope | | (MEF) | MEF | matrigel | (FES22,29,30) |
| LN type 1 | 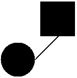 | 87% | | | + |
| SSEA-3 | 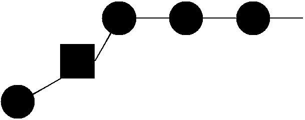 | 74% | | | + |
| SSEA-4 | 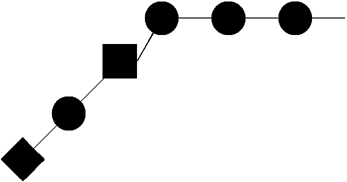 | 23% | | | + |
| Tra-1-60 | 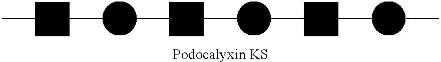 | 47% | 2% | 22% | |

TABLE 19
TLC blot of human embryonic stem cells.

| | | | | | | Cell |
|---|---|---|---|---|---|---|
| | | | | | FACS | Staining |
| epitope | | FES29 | FES30 | FES61 | (FES29) | FES22,29,30 |
| LN type 1 | 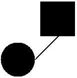 | − | − | − | + | + |
| asialo GM1 | 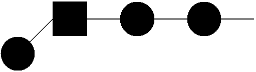 | + | − | − | | |
| SSEA-3 | 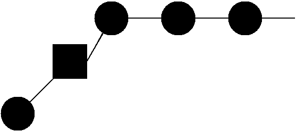 | − | − | − | + | + |
| SSEA-4 | 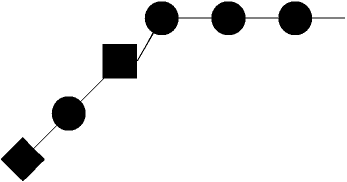 | − | − | + | + | + |
| Galβ1-3GalNAc | 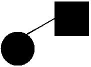 | − | − | − | | |
| asialo GM2 | 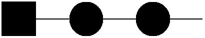 | + | − | − | | |
| globoside | 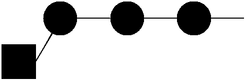 | − | − | − | | +/− |
| Forssman | 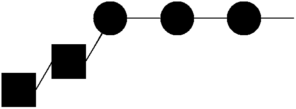 | + | + | + | | − |
| H (1) | 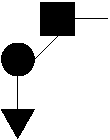 | − | − | − | | + |
| globo H | 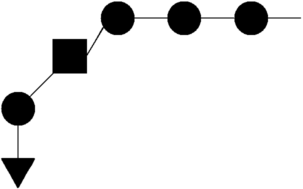 | − | − | − | | +/− |
| H (2) | 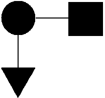 | − | − | − | | + |
| Ley | 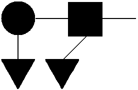 | − | − | − | | ? |
| Leb | 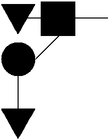 | − | − | − | | +/− |
| Lea | 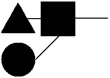 | − | − | − | | − |

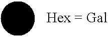

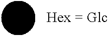

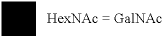

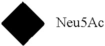

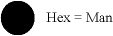

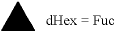

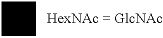

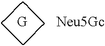

TLC blot of human embryonic stem cells. Experiments with low amounts of Sample, + indicates potential reactivity, − not done or need experiments, 2 columns on right for comparison. Monosacharide symbols below and with Table 14, reducing end on the right.

TABLE 20

| Code | Producer code | Clone | Specificity | host/isotype |
|---|---|---|---|---|
| GF 279 | Abcam ab3352 | K21 | Lewis c, LacNAc (LN) Type 1 | mouse/IgM |
| GF 280 | Glycotope | MAB-S301 | TF-antigen (Galβ3GalNAc) | |
| | | (Nemod TF2) | | |
| GF 281 | Glycotope | MAB-S305 | TF-antigen (Galβ3GalNAc) | Mouse IgG1 |
| | | (A68-E/E3) | | |
| GF 283 | Acris DM3122 | 2-25LE | Lewis b (Leb) | mouse/IgG |
| GF 284 | Acris DM3015 | B393 | H Type 2 H (2) | mouse/IgM |
| GF 285 | Acris DM3014 | B389 | H Type 2, Lewis b, Lewis y | mouse/IgG1 |
| GF 286 | Acris BM258P | BRIC 231 | H Type 2, H (2) | mouse/IgG1 |
| GF 287 | Abcam ab3355 | 17-206 | H Type 1, H (1) | mouse/IgG3 |
| GF 288 | Glycotope MAB-S206 | A69-A/E8 | Globo H | mouse/IgM |
| GF 403 | | | | |
| GF 289 | Glycotope MAB-S201 | A70-C/C8 | Lewis y (Ley) | mouse/IgM |
| GF 290 | Glycotope MAB-S204 | A51-B/A6 | H type 2, H (2) | mouse/IgA |
| GF 304 | Chemicon CBL205 | PR5C5 | Lewis a | |
| GF 305 | Chemicon CBL144 | 28 | Lewis x (Lex) | |
| GF 307 | Chemicon MAB2096 | KM93 | Sialyl Lewis x (Slex) | |
| GF 353 | Chemicon MAB4303 | MC-631 | SSEA-3 | |
| GF 366 | Abcam ab23949 | polyclonal | Gb4, globoside | rabbit |
| GF 367 | Acris | SM1160P | Gb3 globotriose | |
| GF 368 | Leiden University | 259-2A1 | LacdiNAc | mouse/IgG3 |
| GF 369 | Leiden University | 273-3F2 | LacdiNAc | mouse/IgM |
| GF 370 | Leiden University | 290-2E6 | α3-fucosyl-LacdiNAc | mouse/IgM |
| GF 371 | Leiden University | 291-3E9 | α3-fucosyl-LacdiNAc | |
| GF 372 | Acris | B35.1 | Sialyl-Tn | |
| GF 373 | Acris | DM3184P | PN-15 | |
| GF 305 | Chemicon CBL144 | 28 | Lewis x (Lex) | |
| GF 307 | Chemicon MAB2096 | KM93 | Sialyl Lewis x (Slex) | |
| GF 401 | Acris BM4091 | FOM-1 | Forssman antigen | rat/IgM |
| GF 402 | Leiden University | 100-4G11 | low-mannose N-glycan (low | mouse/IgG |
| GF 418 | Alexis | MBr1 | man) | |
| | | | Globo-H | |

TABLE 21

| Trivial name | Terminal epitope | hESC 1) | EB | st.3 |
|---|---|---|---|---|
| LN type 1, Le^{c} | Galβ3GlcNAc | N+ | 2) | +/− |
| | | O+ | +/− | |
| | | L++ | | |
| Lea | Galβ3(Fucα4)GlcNAc | L+ | +/− | +/− |
| H type 1 | Fucα2Galβ3GlcNAc | L++ | +/− | +/− |
| Leb | Fucα2Galβ3(Fucα4)GlcNAc | + | +/− | +/− |
| sialyl Le^{a} | SAα3Galβ3(Fucα4)GlcNAc | | +/− | +/− |
| α3′-sialyl Le^{c} | SAα3Galβ3GlcNAc | | | |
| LN type 2 | Galβ4GlcNAc | N++ | + | + |
| | | O++ | | |
| | | L+/− | | |
| Le^{x} | Galβ4(Fucα3)GlcNAc | N++ | +/− | +/− |
| | | O+/− | | |
| | | L+/− | | |
| H type 2 | Fucα2Galβ4GlcNAc | N+ | +/− | +/− |
| | | O+/− | | |
| | | L+/− | | |
| Le^{y} | Fucα2Galβ4(Fucα3)GlcNAc | + | +/− | +/− |
| sialyl Le^{x} | SAα3Galβ4(Fucα3)GlcNAc | + | +/− | +/− |
| α3′-sialyl LN | SAα3Galβ4GlcNAc | N++ | N+ | N+ |
| | | O+ | | |
| α6′-sialyl LN | SAα6Galβ4GlcNAc | N+ | N++ | N++ |
| Core 1 | Galβ3GalNAcα | O+ | +/− | +/− |
| H type 3 | Fucα2Galβ3GalNAcα | O+ | +/− | +/− |
| sialyl Core 1 | SAα3Galβ3GalNAcα | O+ | | |
| disialyl Core 1 | SAα3Galβ3(SAα6)GalNAcα | O+ | | |
| type 4 chain | Galβ3GalNAcβ | L+ | +/− | +/− |
| H type 4 | Fucα2Galβ3GalNAcβ | L+ | +/− | +/− |
| α3′-sialyl type 4 | SAα3Galβ3GalNAcβ | L++ | +/− | +/− |
| LacdiNAc | GalNAcβ4GlcNAc | N+ | +/− | +/− |
| Lac | Galβ4Glc | L+ | q | q |
| GlcNAcβ | GlcNAcβ | N+/− | q | q |
| | | L+ | | |
| Tn | GalNAcα | q | | |
| sialyl Tn | SAα6GalNAcα | | | |
| GalNAcβ | GalNAcβ | L+ N+ | q | q |
| poly-LN, i | repeats of Galβ4GlcNAcβ3 | + | q | q |
| poly-LN, I | Galβ4GlcNAcβ3(Galβ4GlcNAcβ6)Gal | L+ | +/− | +/− |

1) Stem cell and differentiated cell types are abbreviated as in other parts of the present document; st.3 indicates stage 3 differentiated, preferentially neuronal-type differentiated cells; adipo/osteo indicates cells differentiated into adipocyte or osteoblast direction from MSC.
2) Occurrence of terminal epitopes in glycoconjugates and/or specifically in N-glycans (N), O-glycans (O), and/or glycosphingolipids (L). Code: q, qualitative data; +/−, low expression; +, common; ++, abundant.

TABLE 22

| | | | |
|---|---|---|---|
| | | hESC | hESC |
| Class | Definition | neutral | sialylatecd |

Examples of glycosphingolipid glycan classification

| | | | |
|---|---|---|---|
| Lac | n_{Hex} = 2 | 1 | 1 |
| Ltri | n_{Hex} = 2 and n_{HexNAc} = 1 | 18 | 25 |
| L1 | n_{Hex} = 3 and n_{HexNAc} = 1 | 46 | 56 |
| L2 | 3 ≦ n_{Hex} ≦ 4 and n_{HexNAc} = 2 | 11 | <1 |
| L3+ | i + 1 ≦ n_{Hex} ≦ i + 2 and n_{HexNAc} = i ≧ 3 | 1 | 1 |
| Gb | n_{Hex} = 4 and n_{HexNAc} = 1 | 20 | 16 |
| O | other types | 23 | 1 |
| F | fucosylated, n_{dHex} ≧ 1 | 43 | 1 |
| T | non-reducing terminal HexNAc, | 27 | 26 |
| | n_{Hex} ≦ n_{HexNAc} + 1 | | |
| SA1 | monosialylated, n_{Neu5Ac} = 1 | | 86 |
| SA2 | disialylated, n_{Neu5Ac} = 2 | | 14 |
| SP | sulphated or phosphorylated, +80 Da | | <1 |

Examples of O-linked glycan classification

| | | | |
|---|---|---|---|
| O1 | n_{Hex} = 1 and n_{HexNAc} = 1 | a) | 43 |
| O2 | n_{Hex} = 2 and n_{HexNAc} = 2 | 53 | 35 |
| O3+ | n_{Hex} = i and n_{HexNAc} = i ≧ 3 | 13 | 13 |
| O | other types | 34 | 9 |
| F | fucosylated, n_{dHex} ≧ 1 | 1 | 64 |
| T | non-reducing terminal HexNAc, | 12 | <1 |
| | n_{Hex} ≦ n_{HexNAc} + 1 | | |
| SA1 | monosialylated, n_{Neu5Ac} = 1 | | 39 |
| SA2 | disialylated, n_{Neu5Ac} = 2 | | 52 |
| SP | sulphated or phosphorylated, +80 Da | | 8 |

a) not included in present quantitative analysis.

TABLE 23

| | hESC |
|---|---|
| Neutral | |
| glycosphingolipid | |
| glycans^{#} | |
| L1 | 1 |
| L2 | 64 |
| L3 | 12 |
| L4 | 1 |
| L5+ | 0.5 |
| Gb | 20 |
| O | 2 |
| fucosylated | 43 |
| α1,2-Fuc | 39 |
| α1,3/4-Fuc | 3 |
| β1,4-Gal | 4 |
| β1,3-Gal | 50 |
| term. HexNAc | 27 |
| Acidic | |
| glycosphingolipid | |
| glycans^{#} | |
| L1 | n.d. |
| L2 | 81 |
| L3 | 0.5 |
| L4 | 0.5 |
| L5+ | 0.5 |
| Gb | 16 |
| O | <0.5 |
| α-NeuAc | 100 |
| α2,3-NeuAc | 81 |
| fucosylated | 1 |
| β1,4-Gal | n.d. |

^{#}Abbreviations: L1-6, glycosphingolipid glycan type Li, wherein n_{HexNAc} + 1 ≦ n_{Hex} ≦ n_{HexNAc} + 2, and i = n_{HexNAc} + 1; Gb, (iso)globopentaose, wherein n_{Hex} = 4 and n_{HexNAc} = 1; term. HexNAc, terminal HexNAc in L1-6, wherein n_{HexNAc} + 1 = n_{Hex}; O, other types;
n.d., not determined.
^{§}Figures indicate percentage of total detected glycan signals.

TABLE 24

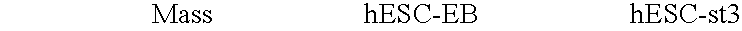

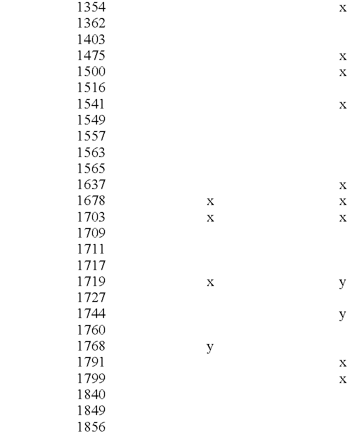

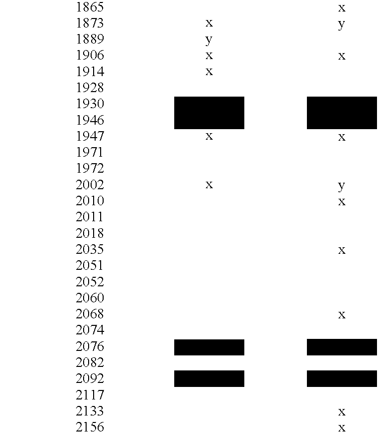

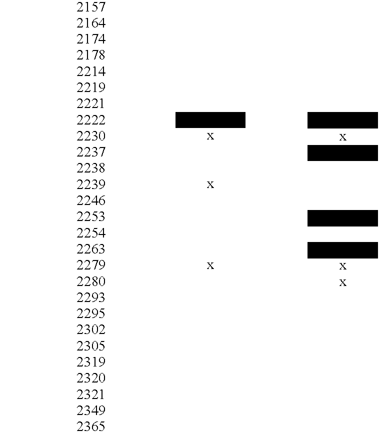

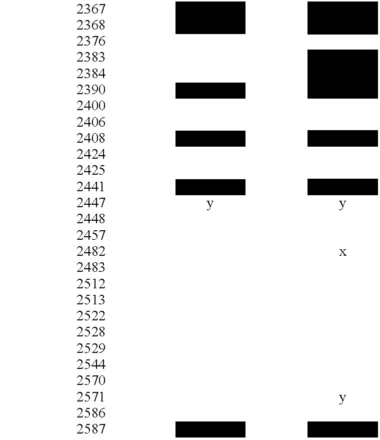

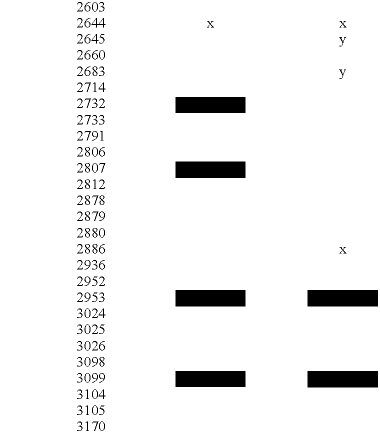

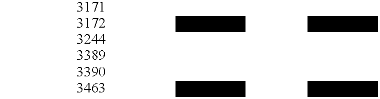

One way ANOVA of acidic glycans from hESC, embryoid bodies and stage 3 stem cells. “x” denotes p-value < 0.05 and “y” equals 0.051 < p-value < 0.099. P-values highlighted with green or light green depict statistically significant down regulation of corresponding mass intensity. Due to low n number p-values < 0.099 were considered to be significant.

TABLE 25

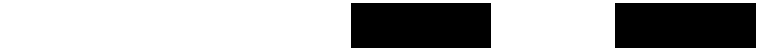

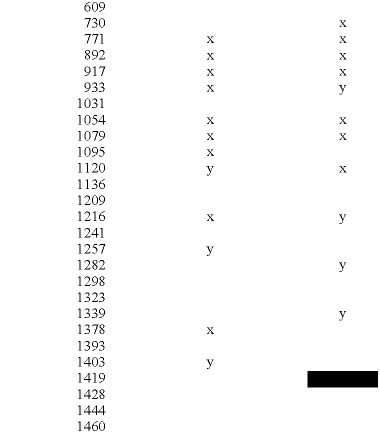

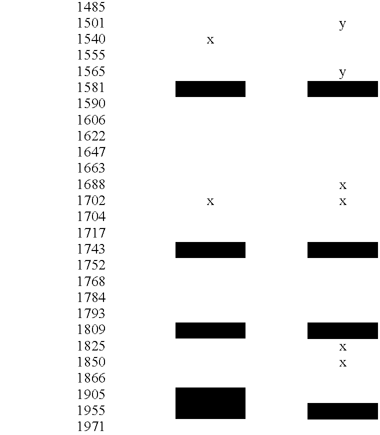

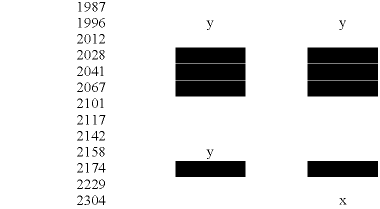

One way ANOVA of N-glycans from hESC, embryoid bodies and stage 3 stem cells. “x” demotes p-value < 0.05 and “y” equals 0.051 < p-value < 0.099. P-values highlighted with green or light green depict statistically significant down regulation of corresponding mass intensity. Due to low n number p-values < 0.099 were considered to be significant.

TABLE 26
Factor loadings for masses derived from acidic glycan of embryonic stem cells.
Total of 13 factors were identified with Eigenvalues >1 but 8 of them explained
approx >5% of all variation.
Factors 1 to 8 explain 24.3%, 12.6%, 11%, 8.1%, 5.9%, 5.6%, 5.1%, and
4.7% of all variation, respectively.

| | Factor 1 | Factor 2 | Factor 3 | Factor 4 | Factor 5 | Factor 6 | Factor 7 | Factor 8 |
|---|---|---|---|---|---|---|---|---|
| 1354 | 0.10 | −0.02 | −0.03 | −0.92 | 0.07 | 0.03 | 0.01 | 0.07 |
| 1362 | 0.10 | 0.11 | −0.05 | −0.01 | 0.11 | −0.06 | −0.48 | 0.02 |
| 1403 | 0.01 | 0.01 | −0.02 | −0.04 | 0.00 | −0.07 | 0.07 | 0.01 |
| 1475 | 0.26 | −0.88 | −0.01 | −0.09 | −0.11 | 0.17 | 0.16 | −0.10 |
| 1500 | 0.28 | −0.37 | 0.10 | −0.68 | −0.25 | 0.27 | 0.01 | −0.08 |
| 1516 | 0.31 | 0.11 | −0.01 | −0.78 | 0.05 | −0.01 | 0.15 | 0.19 |
| 1541 | −0.05 | −0.14 | −0.01 | −0.92 | −0.01 | 0.08 | −0.21 | 0.03 |
| 1549 | −0.06 | 0.15 | 0.19 | 0.12 | 0.11 | 0.07 | 0.06 | 0.04 |
| 1557 | 0.05 | 0.06 | −0.06 | −0.27 | 0.12 | −0.03 | 0.09 | 0.00 |
| 1565 | 0.50 | 0.19 | 0.15 | −0.23 | 0.36 | −0.15 | −0.03 | 0.40 |
| 1637 | 0.29 | −0.80 | 0.02 | −0.15 | −0.08 | 0.20 | 0.08 | −0.13 |
| 1678 | 0.79 | −0.50 | 0.11 | −0.14 | 0.02 | 0.04 | 0.08 | 0.01 |
| 1703 | 0.29 | −0.28 | 0.03 | −0.43 | −0.44 | 0.13 | 0.07 | 0.17 |
| 1711 | 0.02 | −0.20 | −0.22 | 0.53 | −0.02 | 0.35 | −0.02 | 0.10 |
| 1719 | 0.30 | 0.19 | 0.05 | −0.59 | −0.45 | 0.10 | 0.10 | 0.07 |
| 1727 | 0.68 | −0.28 | 0.11 | 0.07 | 0.55 | −0.17 | 0.09 | −0.12 |
| 1744 | 0.33 | −0.25 | 0.04 | −0.51 | −0.06 | 0.09 | −0.15 | 0.25 |
| 1768 | 0.51 | 0.25 | 0.00 | −0.19 | 0.05 | −0.10 | −0.17 | 0.36 |
| 1791 | −0.04 | −0.12 | −0.01 | −0.98 | 0.00 | 0.07 | 0.09 | 0.01 |
| 1799 | 0.16 | −0.90 | −0.03 | −0.02 | 0.12 | 0.10 | −0.21 | 0.20 |
| 1840 | 0.57 | −0.40 | 0.05 | 0.24 | 0.21 | 0.16 | −0.08 | 0.40 |
| 1865 | 0.20 | −0.17 | 0.01 | −0.70 | −0.07 | 0.06 | 0.02 | 0.02 |
| 1873 | 0.85 | −0.25 | 0.12 | −0.04 | −0.04 | 0.29 | −0.01 | −0.05 |
| 1889 | 0.85 | −0.06 | 0.18 | −0.09 | −0.03 | 0.00 | 0.18 | 0.05 |
| 1906 | 0.56 | −0.43 | 0.07 | −0.42 | −0.02 | 0.06 | −0.27 | −0.15 |
| 1914 | 0.74 | −0.14 | 0.17 | −0.16 | −0.07 | 0.34 | −0.12 | −0.19 |
| 1930 | −0.15 | 0.55 | 0.06 | 0.23 | 0.30 | −0.28 | 0.03 | 0.25 |
| 1946 | 0.04 | 0.27 | 0.20 | 0.20 | 0.00 | −0.40 | 0.01 | 0.15 |
| 1947 | 0.44 | −0.34 | 0.03 | −0.38 | −0.06 | 0.16 | −0.09 | −0.28 |
| 2002 | 0.77 | −0.30 | 0.08 | 0.00 | −0.06 | 0.23 | 0.09 | −0.05 |
| 2010 | 0.21 | −0.14 | −0.03 | −0.77 | 0.11 | 0.07 | −0.03 | 0.09 |
| 2011 | 0.12 | 0.00 | 0.20 | 0.07 | −0.73 | −0.10 | −0.13 | 0.05 |
| 2018 | 0.37 | 0.31 | −0.05 | 0.07 | 0.22 | −0.17 | 0.47 | 0.11 |
| 2035 | 0.56 | −0.41 | 0.00 | −0.19 | 0.22 | 0.09 | 0.09 | 0.38 |
| 2052 | 0.62 | −0.31 | 0.16 | −0.03 | −0.09 | 0.33 | 0.01 | −0.10 |
| 2068 | 0.35 | −0.53 | 0.01 | −0.60 | 0.13 | 0.12 | −0.13 | 0.28 |
| 2076 | −0.31 | 0.62 | 0.04 | 0.44 | 0.29 | −0.04 | −0.14 | 0.14 |
| 2092 | −0.08 | 0.52 | 0.47 | 0.44 | −0.04 | −0.24 | −0.09 | −0.06 |
| 2117 | 0.25 | −0.08 | 0.07 | 0.52 | −0.12 | 0.31 | −0.04 | −0.33 |
| 2133 | 0.39 | −0.69 | −0.06 | −0.23 | 0.33 | −0.23 | −0.05 | −0.11 |
| 2156 | 0.33 | −0.14 | 0.04 | −0.79 | 0.04 | 0.04 | 0.06 | −0.03 |
| 2157 | −0.15 | −0.05 | 0.38 | 0.17 | 0.03 | −0.07 | 0.30 | 0.32 |
| 2164 | 0.22 | 0.22 | 0.13 | −0.14 | −0.12 | −0.49 | −0.53 | 0.29 |
| 2221 | −0.19 | 0.21 | −0.86 | 0.19 | 0.12 | −0.16 | 0.09 | 0.06 |
| 2222 | −0.52 | 0.27 | 0.63 | 0.33 | 0.03 | 0.02 | 0.09 | 0.04 |
| 2230 | 0.25 | −0.10 | 0.07 | −0.65 | −0.43 | 0.19 | −0.14 | 0.08 |
| 2237 | 0.12 | 0.30 | 0.12 | 0.22 | 0.18 | −0.40 | 0.04 | −0.34 |
| 2238 | −0.23 | −0.06 | 0.63 | 0.09 | −0.34 | 0.56 | 0.16 | 0.10 |
| 2239 | 0.18 | 0.03 | 0.06 | 0.12 | −0.31 | 0.16 | −0.44 | −0.35 |
| 2246 | −0.01 | −0.01 | −0.03 | −0.72 | 0.09 | 0.04 | 0.44 | −0.09 |
| 2253 | −0.01 | 0.20 | 0.07 | 0.09 | 0.03 | −0.38 | 0.03 | 0.03 |
| 2254 | −0.20 | 0.01 | 0.07 | 0.05 | −0.11 | −0.91 | −0.02 | 0.00 |
| 2263 | −0.12 | −0.14 | 0.53 | 0.39 | −0.11 | 0.11 | 0.11 | −0.20 |
| 2279 | 0.12 | −0.35 | −0.77 | 0.03 | 0.11 | 0.22 | −0.16 | −0.15 |
| 2280 | 0.22 | −0.44 | −0.65 | 0.07 | 0.34 | −0.04 | 0.11 | 0.10 |
| 2295 | 0.29 | 0.42 | 0.23 | 0.02 | 0.20 | −0.18 | −0.52 | −0.31 |
| 2321 | 0.07 | −0.02 | 0.13 | 0.02 | −0.86 | −0.30 | 0.00 | −0.09 |
| 2367 | −0.65 | 0.44 | −0.21 | 0.44 | 0.17 | −0.02 | 0.14 | 0.10 |
| 2368 | −0.31 | 0.27 | 0.57 | 0.20 | 0.32 | −0.33 | 0.18 | −0.23 |
| 2383 | −0.01 | 0.19 | 0.18 | 0.18 | −0.02 | −0.67 | −0.15 | 0.15 |
| 2384 | 0.10 | 0.22 | 0.17 | 0.16 | −0.49 | −0.08 | −0.01 | 0.06 |
| 2390 | −0.31 | 0.23 | 0.41 | 0.10 | 0.12 | −0.30 | −0.09 | 0.17 |
| 2400 | 0.11 | −0.02 | 0.04 | 0.21 | −0.36 | 0.10 | 0.08 | −0.85 |
| 2408 | −0.52 | 0.19 | 0.54 | 0.32 | −0.22 | 0.00 | 0.12 | 0.13 |
| 2425 | 0.09 | −0.39 | 0.54 | 0.20 | −0.24 | 0.22 | 0.12 | −0.29 |
| 2441 | −0.77 | 0.15 | −0.09 | 0.48 | 0.05 | 0.19 | −0.05 | −0.06 |
| 2447 | 0.30 | 0.23 | 0.03 | −0.68 | 0.10 | 0.07 | −0.20 | 0.19 |
| 2448 | 0.26 | 0.15 | −0.04 | −0.30 | 0.12 | −0.02 | −0.09 | 0.16 |
| 2482 | 0.34 | −0.74 | 0.03 | −0.18 | −0.25 | 0.22 | 0.10 | −0.12 |
| 2512 | 0.07 | 0.07 | −0.04 | −0.03 | 0.06 | −0.08 | −0.25 | 0.02 |
| 2513 | 0.10 | 0.12 | −0.04 | 0.01 | 0.13 | −0.03 | −0.59 | 0.02 |
| 2521 | 0.30 | −0.14 | 0.13 | −0.35 | −0.26 | −0.12 | 0.00 | 0.26 |
| 2522 | 0.09 | −0.01 | −0.02 | −0.19 | −0.12 | 0.06 | 0.02 | −0.01 |
| 2528 | −0.15 | 0.05 | 0.05 | 0.05 | −0.05 | −0.88 | −0.24 | 0.00 |
| 2529 | 0.34 | 0.18 | 0.02 | 0.09 | −0.03 | 0.02 | −0.10 | 0.25 |
| 2544 | −0.20 | 0.01 | 0.07 | 0.04 | −0.11 | −0.91 | −0.02 | 0.00 |
| 2570 | 0.00 | 0.06 | −0.74 | 0.10 | 0.10 | −0.12 | −0.11 | −0.12 |
| 2571 | −0.14 | 0.08 | −0.70 | −0.18 | −0.28 | 0.18 | 0.36 | −0.35 |
| 2586 | 0.15 | 0.24 | 0.07 | 0.02 | 0.00 | 0.04 | −0.16 | 0.08 |
| 2587 | −0.55 | 0.15 | 0.67 | 0.21 | 0.01 | 0.02 | 0.13 | −0.02 |
| 2603 | 0.02 | −0.02 | 0.07 | 0.14 | −0.90 | 0.13 | 0.20 | −0.13 |
| 2644 | −0.07 | −0.33 | −0.86 | −0.06 | −0.05 | 0.23 | 0.00 | −0.05 |
| 2645 | −0.22 | −0.03 | −0.90 | 0.16 | 0.07 | 0.10 | 0.05 | 0.01 |
| 2660 | −0.07 | 0.14 | 0.20 | 0.13 | 0.11 | 0.09 | 0.04 | 0.03 |
| 2683 | 0.25 | −0.37 | 0.04 | −0.23 | −0.36 | 0.21 | −0.15 | 0.18 |
| 2714 | 0.14 | −0.70 | −0.08 | 0.26 | 0.23 | −0.01 | 0.18 | 0.20 |
| 2732 | −0.68 | 0.32 | −0.53 | 0.09 | 0.12 | 0.01 | 0.04 | 0.24 |
| 2733 | −0.02 | 0.06 | 0.36 | 0.27 | 0.53 | 0.25 | 0.31 | −0.07 |
| 2807 | −0.80 | −0.04 | −0.18 | 0.23 | 0.08 | 0.18 | 0.32 | −0.24 |
| 2878 | 0.20 | −0.04 | 0.02 | 0.23 | 0.22 | 0.13 | 0.25 | 0.14 |
| 2879 | −0.03 | 0.04 | 0.02 | 0.09 | 0.07 | −0.61 | −0.15 | −0.50 |
| 2880 | −0.68 | 0.07 | 0.46 | 0.16 | 0.18 | 0.19 | 0.13 | 0.14 |
| 2886 | 0.13 | −0.41 | −0.01 | −0.58 | 0.15 | 0.10 | 0.07 | 0.17 |
| 2936 | −0.26 | 0.24 | −0.87 | 0.16 | 0.05 | 0.04 | 0.16 | 0.13 |
| 2953 | −0.59 | 0.12 | 0.44 | 0.21 | 0.09 | −0.49 | 0.07 | 0.10 |
| 3024 | 0.19 | 0.21 | −0.04 | −0.48 | 0.19 | −0.31 | 0.64 | 0.01 |
| 3025 | 0.09 | 0.21 | 0.02 | 0.10 | 0.07 | −0.82 | 0.29 | 0.07 |
| 3098 | −0.35 | 0.20 | −0.86 | 0.17 | 0.01 | 0.14 | 0.05 | 0.10 |
| 3099 | −0.74 | 0.09 | 0.48 | 0.12 | 0.11 | −0.35 | 0.02 | 0.09 |
| 3170 | 0.12 | −0.01 | −0.01 | 0.14 | 0.19 | 0.02 | −0.04 | −0.90 |
| 3171 | 0.01 | 0.01 | −0.02 | −0.04 | 0.00 | −0.07 | 0.07 | 0.01 |
| 3172 | −0.72 | 0.07 | 0.47 | 0.18 | 0.13 | −0.16 | 0.11 | 0.13 |
| 3390 | −0.01 | 0.15 | 0.05 | 0.09 | 0.01 | −0.92 | 0.18 | 0.05 |
| 3463 | −0.08 | 0.20 | 0.13 | 0.15 | 0.01 | −0.29 | 0.00 | 0.01 |
| Expl. Var | 13.78 | 9.49 | 10.82 | 12.50 | 5.86 | 8.57 | 3.89 | 4.66 |
| Prp. Totl | 0.13 | 0.09 | 0.10 | 0.12 | 0.06 | 0.08 | 0.04 | 0.04 |

TABLE 27
Communalities for masses derived from acidic glycan
of embryonic stem cells.
COMMUNALITIES

| | From | From | From | From | | | | |
|---|---|---|---|---|---|---|---|---|
| | 1 | 2 | 3 | 4 | From | From | From | From |
| | Fac- | Fac- | Fac- | Fac- | 5 | 6 | 7 | 8 |
| | tor | tors | tors | tors | Factors | Factors | Factors | Factors |
| 1354 | 0.009 | 0.009 | 0.010 | 0.860 | 0.865 | 0.866 | 0.866 | 0.870 |
| 1362 | 0.010 | 0.022 | 0.024 | 0.024 | 0.037 | 0.041 | 0.276 | 0.276 |
| 1403 | 0.000 | 0.000 | 0.001 | 0.003 | 0.003 | 0.008 | 0.012 | 0.012 |
| 1475 | 0.067 | 0.845 | 0.845 | 0.854 | 0.866 | 0.895 | 0.920 | 0.931 |
| 1500 | 0.076 | 0.216 | 0.226 | 0.692 | 0.753 | 0.826 | 0.827 | 0.833 |
| 1516 | 0.093 | 0.105 | 0.105 | 0.708 | 0.710 | 0.710 | 0.732 | 0.769 |
| 1541 | 0.002 | 0.022 | 0.022 | 0.876 | 0.876 | 0.882 | 0.927 | 0.928 |
| 1549 | 0.004 | 0.025 | 0.062 | 0.076 | 0.088 | 0.093 | 0.096 | 0.097 |
| 1557 | 0.003 | 0.007 | 0.010 | 0.081 | 0.095 | 0.096 | 0.104 | 0.104 |
| 1565 | 0.249 | 0.284 | 0.308 | 0.360 | 0.488 | 0.510 | 0.510 | 0.674 |
| 1637 | 0.086 | 0.732 | 0.732 | 0.755 | 0.761 | 0.801 | 0.807 | 0.823 |
| 1678 | 0.626 | 0.871 | 0.883 | 0.902 | 0.902 | 0.904 | 0.911 | 0.911 |
| 1703 | 0.085 | 0.163 | 0.164 | 0.351 | 0.548 | 0.564 | 0.569 | 0.599 |
| 1711 | 0.000 | 0.039 | 0.088 | 0.373 | 0.374 | 0.495 | 0.495 | 0.505 |
| 1719 | 0.088 | 0.126 | 0.128 | 0.482 | 0.684 | 0.694 | 0.704 | 0.708 |
| 1727 | 0.469 | 0.545 | 0.556 | 0.562 | 0.860 | 0.890 | 0.898 | 0.914 |
| 1744 | 0.108 | 0.170 | 0.172 | 0.437 | 0.440 | 0.448 | 0.470 | 0.530 |
| 1768 | 0.263 | 0.327 | 0.327 | 0.363 | 0.365 | 0.374 | 0.404 | 0.533 |
| 1791 | 0.001 | 0.016 | 0.016 | 0.968 | 0.968 | 0.973 | 0.982 | 0.982 |
| 1799 | 0.024 | 0.832 | 0.833 | 0.834 | 0.849 | 0.859 | 0.903 | 0.942 |
| 1840 | 0.326 | 0.486 | 0.489 | 0.546 | 0.591 | 0.618 | 0.625 | 0.785 |
| 1865 | 0.042 | 0.071 | 0.071 | 0.564 | 0.569 | 0.572 | 0.572 | 0.573 |
| 1873 | 0.714 | 0.776 | 0.791 | 0.793 | 0.795 | 0.880 | 0.880 | 0.882 |
| 1889 | 0.726 | 0.730 | 0.761 | 0.769 | 0.770 | 0.770 | 0.803 | 0.806 |
| 1906 | 0.319 | 0.507 | 0.513 | 0.690 | 0.690 | 0.694 | 0.766 | 0.787 |
| 1914 | 0.549 | 0.568 | 0.596 | 0.621 | 0.625 | 0.742 | 0.758 | 0.795 |
| 1930 | 0.022 | 0.326 | 0.330 | 0.384 | 0.471 | 0.552 | 0.553 | 0.616 |
| 1946 | 0.001 | 0.075 | 0.114 | 0.154 | 0.154 | 0.315 | 0.315 | 0.338 |
| 1947 | 0.193 | 0.312 | 0.313 | 0.455 | 0.459 | 0.484 | 0.492 | 0.569 |
| 2002 | 0.591 | 0.682 | 0.688 | 0.688 | 0.692 | 0.745 | 0.753 | 0.755 |
| 2010 | 0.045 | 0.065 | 0.066 | 0.666 | 0.678 | 0.683 | 0.685 | 0.694 |
| 2011 | 0.015 | 0.015 | 0.054 | 0.059 | 0.595 | 0.605 | 0.621 | 0.623 |
| 2018 | 0.136 | 0.231 | 0.234 | 0.240 | 0.286 | 0.313 | 0.530 | 0.542 |
| 2035 | 0.313 | 0.477 | 0.477 | 0.514 | 0.560 | 0.568 | 0.577 | 0.720 |
| 2052 | 0.378 | 0.471 | 0.498 | 0.498 | 0.507 | 0.615 | 0.615 | 0.625 |
| 2068 | 0.123 | 0.402 | 0.402 | 0.758 | 0.775 | 0.788 | 0.807 | 0.886 |
| 2076 | 0.097 | 0.485 | 0.487 | 0.677 | 0.760 | 0.761 | 0.782 | 0.801 |
| 2092 | 0.007 | 0.282 | 0.506 | 0.701 | 0.702 | 0.759 | 0.767 | 0.771 |
| 2117 | 0.064 | 0.069 | 0.074 | 0.343 | 0.357 | 0.455 | 0.457 | 0.568 |
| 2133 | 0.156 | 0.631 | 0.634 | 0.689 | 0.798 | 0.849 | 0.852 | 0.865 |
| 2156 | 0.108 | 0.129 | 0.131 | 0.755 | 0.757 | 0.759 | 0.762 | 0.763 |
| 2157 | 0.021 | 0.024 | 0.171 | 0.201 | 0.202 | 0.207 | 0.296 | 0.401 |
| 2164 | 0.048 | 0.096 | 0.113 | 0.134 | 0.148 | 0.387 | 0.669 | 0.754 |
| 2221 | 0.038 | 0.083 | 0.821 | 0.858 | 0.874 | 0.898 | 0.906 | 0.909 |
| 2222 | 0.269 | 0.343 | 0.735 | 0.847 | 0.848 | 0.848 | 0.856 | 0.858 |
| 2230 | 0.063 | 0.073 | 0.078 | 0.497 | 0.684 | 0.720 | 0.741 | 0.747 |
| 2237 | 0.014 | 0.103 | 0.117 | 0.166 | 0.197 | 0.360 | 0.361 | 0.477 |
| 2238 | 0.054 | 0.057 | 0.451 | 0.460 | 0.578 | 0.893 | 0.920 | 0.931 |
| 2239 | 0.033 | 0.034 | 0.038 | 0.052 | 0.145 | 0.171 | 0.365 | 0.485 |
| 2246 | 0.000 | 0.000 | 0.001 | 0.515 | 0.524 | 0.525 | 0.721 | 0.729 |
| 2253 | 0.000 | 0.041 | 0.047 | 0.055 | 0.056 | 0.201 | 0.202 | 0.203 |
| 2254 | 0.040 | 0.040 | 0.045 | 0.047 | 0.058 | 0.893 | 0.893 | 0.893 |
| 2263 | 0.015 | 0.033 | 0.312 | 0.461 | 0.473 | 0.486 | 0.498 | 0.537 |
| 2279 | 0.014 | 0.134 | 0.733 | 0.734 | 0.746 | 0.795 | 0.822 | 0.845 |
| 2280 | 0.047 | 0.240 | 0.667 | 0.671 | 0.788 | 0.790 | 0.801 | 0.811 |
| 2295 | 0.082 | 0.254 | 0.307 | 0.308 | 0.347 | 0.379 | 0.647 | 0.742 |
| 2321 | 0.004 | 0.005 | 0.022 | 0.022 | 0.761 | 0.851 | 0.851 | 0.859 |
| 2367 | 0.421 | 0.612 | 0.658 | 0.855 | 0.885 | 0.886 | 0.906 | 0.915 |
| 2368 | 0.094 | 0.166 | 0.487 | 0.526 | 0.630 | 0.742 | 0.774 | 0.827 |
| 2383 | 0.000 | 0.037 | 0.071 | 0.103 | 0.103 | 0.548 | 0.569 | 0.591 |
| 2384 | 0.010 | 0.058 | 0.086 | 0.112 | 0.353 | 0.359 | 0.359 | 0.362 |
| 2390 | 0.097 | 0.149 | 0.315 | 0.324 | 0.337 | 0.428 | 0.436 | 0.463 |
| 2400 | 0.012 | 0.012 | 0.013 | 0.056 | 0.184 | 0.194 | 0.200 | 0.919 |
| 2408 | 0.275 | 0.311 | 0.603 | 0.705 | 0.755 | 0.755 | 0.769 | 0.787 |
| 2425 | 0.008 | 0.158 | 0.447 | 0.487 | 0.544 | 0.592 | 0.606 | 0.689 |
| 2441 | 0.591 | 0.614 | 0.623 | 0.857 | 0.859 | 0.894 | 0.897 | 0.900 |
| 2447 | 0.093 | 0.148 | 0.149 | 0.618 | 0.627 | 0.632 | 0.672 | 0.706 |
| 2448 | 0.068 | 0.091 | 0.093 | 0.182 | 0.195 | 0.196 | 0.205 | 0.229 |
| 2482 | 0.113 | 0.654 | 0.655 | 0.688 | 0.750 | 0.798 | 0.807 | 0.821 |
| 2512 | 0.004 | 0.010 | 0.011 | 0.012 | 0.016 | 0.022 | 0.082 | 0.083 |
| 2513 | 0.011 | 0.024 | 0.026 | 0.026 | 0.043 | 0.043 | 0.390 | 0.391 |
| 2521 | 0.091 | 0.110 | 0.125 | 0.245 | 0.311 | 0.327 | 0.327 | 0.393 |
| 2522 | 0.008 | 0.008 | 0.009 | 0.047 | 0.062 | 0.065 | 0.066 | 0.066 |
| 2528 | 0.023 | 0.026 | 0.029 | 0.031 | 0.034 | 0.814 | 0.872 | 0.872 |
| 2529 | 0.117 | 0.151 | 0.151 | 0.160 | 0.160 | 0.161 | 0.171 | 0.233 |
| 2544 | 0.039 | 0.039 | 0.044 | 0.046 | 0.057 | 0.883 | 0.883 | 0.883 |
| 2570 | 0.000 | 0.004 | 0.557 | 0.566 | 0.577 | 0.590 | 0.603 | 0.618 |
| 2571 | 0.019 | 0.026 | 0.510 | 0.541 | 0.618 | 0.650 | 0.777 | 0.901 |
| 2586 | 0.022 | 0.078 | 0.083 | 0.083 | 0.083 | 0.085 | 0.111 | 0.118 |
| 2587 | 0.298 | 0.320 | 0.774 | 0.818 | 0.818 | 0.818 | 0.835 | 0.836 |
| 2603 | 0.000 | 0.001 | 0.006 | 0.027 | 0.838 | 0.854 | 0.896 | 0.915 |
| 2644 | 0.005 | 0.111 | 0.845 | 0.849 | 0.851 | 0.904 | 0.904 | 0.906 |
| 2645 | 0.049 | 0.050 | 0.867 | 0.892 | 0.897 | 0.908 | 0.910 | 0.910 |
| 2660 | 0.005 | 0.025 | 0.065 | 0.083 | 0.096 | 0.103 | 0.105 | 0.106 |
| 2683 | 0.062 | 0.198 | 0.199 | 0.250 | 0.380 | 0.424 | 0.447 | 0.481 |
| 2714 | 0.020 | 0.513 | 0.519 | 0.586 | 0.639 | 0.639 | 0.671 | 0.710 |
| 2732 | 0.460 | 0.563 | 0.839 | 0.848 | 0.863 | 0.863 | 0.865 | 0.922 |
| 2733 | 0.000 | 0.004 | 0.135 | 0.207 | 0.489 | 0.552 | 0.646 | 0.651 |
| 2807 | 0.632 | 0.634 | 0.666 | 0.720 | 0.727 | 0.760 | 0.864 | 0.920 |
| 2878 | 0.041 | 0.043 | 0.043 | 0.094 | 0.143 | 0.160 | 0.223 | 0.241 |
| 2879 | 0.001 | 0.002 | 0.003 | 0.011 | 0.016 | 0.384 | 0.407 | 0.659 |
| 2880 | 0.457 | 0.463 | 0.674 | 0.701 | 0.734 | 0.770 | 0.788 | 0.807 |
| 2886 | 0.017 | 0.189 | 0.189 | 0.522 | 0.543 | 0.553 | 0.558 | 0.586 |
| 2936 | 0.067 | 0.123 | 0.885 | 0.911 | 0.913 | 0.915 | 0.942 | 0.957 |
| 2953 | 0.348 | 0.363 | 0.557 | 0.602 | 0.611 | 0.852 | 0.856 | 0.866 |
| 3024 | 0.037 | 0.079 | 0.081 | 0.314 | 0.350 | 0.448 | 0.862 | 0.862 |
| 3025 | 0.008 | 0.055 | 0.055 | 0.065 | 0.069 | 0.748 | 0.830 | 0.835 |
| 3098 | 0.123 | 0.165 | 0.897 | 0.927 | 0.928 | 0.946 | 0.948 | 0.959 |
| 3099 | 0.552 | 0.560 | 0.791 | 0.806 | 0.819 | 0.945 | 0.945 | 0.954 |
| 3170 | 0.013 | 0.013 | 0.013 | 0.033 | 0.071 | 0.072 | 0.073 | 0.888 |
| 3171 | 0.000 | 0.000 | 0.001 | 0.003 | 0.003 | 0.008 | 0.012 | 0.012 |
| 3172 | 0.523 | 0.527 | 0.747 | 0.779 | 0.796 | 0.823 | 0.835 | 0.851 |
| 3390 | 0.000 | 0.023 | 0.025 | 0.034 | 0.034 | 0.878 | 0.911 | 0.913 |
| 3463 | 0.006 | 0.044 | 0.060 | 0.081 | 0.081 | 0.168 | 0.168 | 0.168 |

TABLE 28
Factor loadings for masses derived from neutral N-glycan
of embryonic stem cells.
Factors representing Eigenvalues > 1 are shown.
Factors 1 to 7 explain 26.30%, 15.30%, 11.04%, 10.09%, 7.59%,
7.27% and 4.45% of all variation, respectively.
hESC Varimax normalised

| | % explained | | | | | | |
|---|---|---|---|---|---|---|---|
| | 26.30 | 15.30 | 11.04 | 10.09 | 7.59 | 7.27 | 4.45 |
| | Factor 1 | Factor 2 | Factor 3 | Factor 4 | Factor 5 | Factor 6 | Factor 7 |
| 609 | −0.79 | 0.00 | 0.14 | 0.03 | −0.30 | 0.11 | −0.10 |
| 730 | 0.28 | 0.06 | −0.21 | 0.40 | 0.77 | −0.10 | 0.00 |
| 771 | 0.72 | 0.07 | −0.34 | 0.05 | 0.47 | 0.09 | −0.05 |
| 892 | 0.81 | −0.05 | −0.14 | 0.20 | 0.42 | −0.11 | 0.08 |
| 917 | 0.46 | 0.02 | −0.62 | −0.19 | 0.46 | 0.34 | −0.02 |
| 933 | 0.81 | 0.01 | −0.34 | −0.15 | 0.01 | 0.31 | 0.20 |
| 1031 | 0.13 | 0.02 | −0.03 | −0.04 | 0.69 | −0.06 | 0.07 |
| 1054 | 0.78 | −0.03 | −0.21 | 0.06 | 0.21 | −0.05 | 0.04 |
| [symbol] | 0.51 | −0.21 | −0.60 | −0.20 | 0.35 | 0.37 | 0.12 |
| 1095 | 0.78 | 0.03 | −0.13 | 0.13 | −0.08 | 0.46 | 0.21 |
| 1120 | 0.37 | 0.16 | −0.88 | 0.16 | 0.05 | 0.02 | −0.14 |
| 1136 | 0.14 | −0.16 | 0.11 | 0.82 | −0.07 | −0.41 | 0.03 |
| 1209 | 0.06 | −0.01 | −0.05 | 0.89 | 0.03 | −0.18 | 0.06 |
| 1216 | 0.86 | 0.20 | 0.08 | 0.26 | 0.03 | 0.15 | 0.04 |
| 1241 | 0.24 | 0.12 | −0.71 | 0.09 | −0.05 | 0.56 | 0.18 |
| 1257 | 0.11 | −0.52 | 0.08 | −0.25 | 0.13 | [symbol] | −0.25 |
| 1282 | 0.13 | −0.14 | −0.91 | 0.18 | 0.07 | −0.09 | 0.05 |
| 1298 | 0.09 | −0.38 | 0.78 | 0.10 | −0.23 | 0.11 | 0.09 |
| 1339 | 0.25 | 0.10 | −0.81 | −0.27 | 0.17 | −0.12 | −0.17 |
| 1378 | 0.86 | 0.22 | −0.12 | 0.10 | −0.30 | 0.13 | −0.07 |
| [symbol] | −0.46 | 0.05 | 0.17 | 0.24 | −0.05 | 0.58 | −0.04 |
| 1403 | 0.31 | −0.09 | −0.81 | −0.16 | 0.12 | 0.34 | −0.02 |
| [symbol] | −0.30 | 0.43 | 0.09 | −0.47 | −0.11 | 0.56 | 0.19 |
| 1444 | −0.14 | 0.03 | −0.61 | 0.01 | −0.54 | 0.17 | −0.23 |
| 1460 | 0.12 | −0.77 | 0.51 | −0.11 | −0.22 | −0.13 | −0.15 |
| 1485 | −0.17 | −0.80 | 0.27 | 0.06 | 0.32 | −0.02 | 0.14 |
| 1501 | 0.32 | 0.10 | −0.82 | −0.23 | 0.25 | −0.19 | −0.12 |
| 1540 | 0.82 | 0.17 | 0.23 | 0.22 | −0.24 | −0.06 | −0.31 |
| [symbol] | −0.08 | 0.23 | 0.18 | 0.00 | 0.33 | 0.28 | 0.38 |
| 1565 | 0.11 | −0.12 | −0.20 | 0.10 | 0.79 | 0.42 | −0.14 |
| [symbol] | −0.66 | 0.58 | 0.03 | −0.20 | 0.09 | 0.21 | 0.09 |
| 1590 | 0.09 | 0.33 | 0.12 | 0.67 | 0.12 | 0.27 | 0.08 |
| 1606 | −0.15 | −0.81 | 0.10 | 0.08 | −0.01 | 0.25 | 0.44 |
| 1622 | 0.13 | −0.79 | 0.42 | −0.10 | −0.23 | −0.08 | −0.25 |
| 1647 | −0.45 | −0.67 | 0.18 | 0.22 | 0.02 | 0.38 | 0.30 |
| [symbol] | −0.50 | −0.42 | 0.23 | 0.17 | −0.52 | −0.26 | −0.06 |
| 1688 | −0.18 | −0.38 | −0.19 | 0.24 | 0.64 | 0.31 | 0.01 |
| 1702 | 0.85 | 0.09 | 0.05 | 0.35 | −0.17 | 0.02 | −0.18 |
| 1704 | 0.00 | −0.88 | −0.02 | −0.18 | −0.21 | 0.00 | 0.32 |
| [symbol] | 0.12 | 0.16 | 0.17 | 0.39 | 0.36 | 0.11 | 0.37 |
| 1743 | [symbol] | 0.37 | 0.32 | −0.08 | 0.08 | −0.39 | −0.12 |
| [symbol] | 0.08 | 0.09 | 0.03 | −0.05 | 0.02 | 0.31 | 0.02 |
| [symbol] | −0.05 | −0.48 | −0.16 | 0.11 | 0.27 | −0.20 | −0.15 |
| 1784 | 0.24 | −0.18 | 0.15 | −0.10 | 0.03 | −0.13 | 0.65 |
| 1793 | 0.30 | 0.36 | −0.72 | 0.03 | −0.13 | −0.14 | −0.17 |
| 1809 | −0.78 | −0.11 | 0.14 | −0.05 | −0.48 | 0.12 | −0.05 |
| 1825 | 0.03 | −0.21 | −0.41 | −0.23 | 0.67 | −0.37 | −0.22 |
| 1850 | 0.02 | −0.90 | −0.19 | 0.17 | 0.24 | 0.09 | −0.03 |
| 1866 | 0.11 | −0.86 | 0.04 | −0.31 | −0.06 | −0.22 | 0.11 |
| 1905 | −0.28 | 0.25 | 0.32 | 0.01 | −0.26 | [symbol] | 0.06 |
| 1955 | −0.83 | 0.32 | 0.17 | 0.20 | −0.07 | −0.02 | −0.17 |
| [symbol] | −0.06 | −0.52 | −0.01 | 0.47 | 0.03 | 0.30 | −0.07 |
| 1987 | 0.24 | 0.16 | −0.67 | 0.03 | −0.25 | −0.46 | −0.19 |
| 1996 | 0.14 | 0.07 | −0.86 | −0.06 | 0.19 | 0.05 | −0.32 |
| 2012 | 0.14 | −0.71 | 0.16 | 0.30 | −0.24 | 0.21 | 0.47 |
| 2028 | −0.73 | 0.11 | 0.35 | 0.07 | −0.32 | 0.10 | 0.33 |
| [symbol] | −0.32 | −0.08 | 0.33 | 0.45 | −0.20 | 0.02 | 0.68 |
| [symbol] | −0.05 | 0.29 | 0.55 | −0.22 | −0.32 | −0.52 | 0.32 |
| [symbol] | −0.37 | 0.47 | −0.40 | 0.10 | −0.41 | 0.35 | −0.06 |
| 2117 | 0.03 | 0.05 | −0.02 | −0.09 | 0.63 | 0.04 | −0.06 |
| [symbol] | 0.31 | 0.17 | 0.63 | −0.15 | −0.21 | −0.07 | −0.46 |
| 2158 | 0.20 | −0.04 | 0.05 | 0.82 | 0.13 | 0.31 | 0.04 |
| 2174 | −0.79 | 0.16 | 0.32 | 0.04 | −0.32 | 0.12 | 0.24 |
| 2229 | −0.04 | −0.37 | 0.22 | 0.24 | −0.19 | −0.07 | 0.79 |
| 2304 | 0.12 | −0.03 | −0.21 | 0.21 | 0.85 | 0.24 | −0.10 |
| [symbol] | −0.57 | 0.42 | 0.11 | 0.46 | −0.12 | 0.00 | 0.08 |
| 2391 | 0.03 | −0.06 | 0.00 | 0.86 | 0.13 | 0.10 | 0.10 |
| 2393 | 0.26 | 0.12 | 0.00 | 0.14 | 0.33 | 0.02 | 0.25 |
| 2466 | 0.05 | −0.07 | −0.07 | 0.85 | −0.05 | −0.27 | 0.02 |

TABLE 29
Communalities for masses derived from neutral N-glycans
of embryonic stem cells.

| | From 1 | From 2 | From 3 | From 4 | From 5 | From 6 | From 7 |
|---|---|---|---|---|---|---|---|
| | Factor | Factors | Factors | Factors | Factors | Factors | Factors |
| 609 | 0.618 | 0.618 | 0.639 | 0.640 | 0.733 | 0.745 | 0.755 |
| 730 | 0.080 | 0.084 | 0.128 | 0.286 | 0.876 | 0.887 | 0.887 |
| 771 | 0.525 | 0.531 | 0.648 | 0.650 | 0.874 | 0.883 | 0.885 |
| 892 | 0.663 | 0.665 | 0.684 | 0.724 | 0.901 | 0.914 | 0.921 |
| 917 | 0.209 | 0.209 | 0.591 | 0.626 | 0.838 | 0.953 | 0.953 |
| 933 | 0.649 | 0.650 | 0.765 | 0.789 | 0.789 | 0.885 | 0.925 |
| 1031 | 0.016 | 0.016 | 0.017 | 0.019 | 0.492 | 0.496 | 0.500 |
| 1054 | 0.605 | 0.606 | 0.648 | 0.651 | 0.696 | 0.698 | 0.699 |
| 1079 | 0.257 | 0.301 | 0.663 | 0.701 | 0.824 | 0.959 | 0.973 |
| 1095 | 0.609 | 0.610 | 0.628 | 0.644 | 0.651 | 0.865 | 0.908 |
| 1120 | 0.136 | 0.162 | 0.936 | 0.962 | 0.965 | 0.965 | 0.985 |
| 1136 | 0.020 | 0.045 | 0.057 | 0.722 | 0.727 | 0.896 | 0.897 |
| 1209 | 0.003 | 0.003 | 0.006 | 0.791 | 0.792 | 0.823 | 0.827 |
| 1216 | 0.741 | 0.780 | 0.786 | 0.853 | 0.854 | 0.875 | 0.877 |
| 1241 | 0.058 | 0.072 | 0.577 | 0.586 | 0.589 | 0.897 | 0.929 |
| 1257 | 0.012 | 0.282 | 0.288 | 0.349 | 0.365 | 0.847 | 0.912 |
| 1282 | 0.017 | 0.037 | 0.862 | 0.895 | 0.901 | 0.909 | 0.911 |
| 1298 | 0.009 | 0.156 | 0.763 | 0.773 | 0.825 | 0.838 | 0.845 |
| 1339 | 0.063 | 0.073 | 0.731 | 0.802 | 0.830 | 0.843 | 0.873 |
| 1378 | 0.736 | 0.783 | 0.797 | 0.808 | 0.901 | 0.919 | 0.924 |
| 1393 | 0.213 | 0.215 | 0.244 | 0.301 | 0.304 | 0.641 | 0.642 |
| 1403 | 0.093 | 0.101 | 0.764 | 0.789 | 0.804 | 0.918 | 0.918 |
| 1419 | 0.093 | 0.280 | 0.288 | 0.510 | 0.522 | 0.831 | 0.868 |
| 1444 | 0.020 | 0.021 | 0.394 | 0.394 | 0.683 | 0.712 | 0.764 |
| 1460 | 0.014 | 0.608 | 0.867 | 0.879 | 0.927 | 0.945 | 0.966 |
| 1485 | 0.029 | 0.661 | 0.732 | 0.736 | 0.835 | 0.835 | 0.854 |
| 1501 | 0.104 | 0.113 | 0.780 | 0.835 | 0.895 | 0.930 | 0.944 |
| 1540 | 0.667 | 0.695 | 0.747 | 0.795 | 0.854 | 0.857 | 0.952 |
| 1555 | 0.007 | 0.059 | 0.093 | 0.093 | 0.203 | 0.283 | 0.424 |
| 1565 | 0.013 | 0.028 | 0.067 | 0.076 | 0.695 | 0.874 | 0.894 |
| 1581 | 0.430 | 0.767 | 0.768 | 0.810 | 0.818 | 0.861 | 0.868 |
| 1590 | 0.007 | 0.118 | 0.132 | 0.583 | 0.597 | 0.672 | 0.679 |
| 1606 | 0.022 | 0.672 | 0.682 | 0.688 | 0.688 | 0.753 | 0.944 |
| 1622 | 0.016 | 0.638 | 0.810 | 0.820 | 0.871 | 0.878 | 0.939 |
| 1647 | 0.198 | 0.647 | 0.678 | 0.728 | 0.728 | 0.871 | 0.961 |
| 1663 | 0.253 | 0.425 | 0.478 | 0.508 | 0.780 | 0.849 | 0.852 |
| 1688 | 0.034 | 0.176 | 0.210 | 0.266 | 0.679 | 0.773 | 0.773 |
| 1702 | 0.730 | 0.738 | 0.740 | 0.865 | 0.895 | 0.896 | 0.927 |
| 1704 | 0.000 | 0.768 | 0.768 | 0.802 | 0.847 | 0.847 | 0.951 |
| 1717 | 0.015 | 0.040 | 0.071 | 0.219 | 0.350 | 0.363 | 0.499 |
| 1743 | 0.554 | 0.689 | 0.789 | 0.796 | 0.802 | 0.957 | 0.970 |
| 1752 | 0.007 | 0.015 | 0.016 | 0.018 | 0.019 | 0.112 | 0.112 |
| 1768 | 0.003 | 0.229 | 0.254 | 0.268 | 0.339 | 0.380 | 0.401 |
| 1784 | 0.057 | 0.089 | 0.111 | 0.122 | 0.123 | 0.141 | 0.559 |
| 1793 | 0.088 | 0.215 | 0.729 | 0.730 | 0.748 | 0.768 | 0.797 |
| 1809 | 0.604 | 0.616 | 0.635 | 0.638 | 0.867 | 0.883 | 0.885 |
| 1825 | 0.001 | 0.045 | 0.212 | 0.266 | 0.714 | 0.852 | 0.901 |
| 1850 | 0.000 | 0.803 | 0.838 | 0.866 | 0.925 | 0.934 | 0.935 |
| 1866 | 0.012 | 0.748 | 0.750 | 0.847 | 0.850 | 0.898 | 0.911 |
| 1905 | 0.077 | 0.139 | 0.244 | 0.244 | 0.310 | 0.952 | 0.955 |
| 1955 | 0.683 | 0.787 | 0.816 | 0.857 | 0.862 | 0.863 | 0.890 |
| 1971 | 0.004 | 0.272 | 0.273 | 0.491 | 0.492 | 0.580 | 0.584 |
| 1987 | 0.059 | 0.084 | 0.536 | 0.537 | 0.598 | 0.806 | 0.842 |
| 1996 | 0.020 | 0.026 | 0.768 | 0.772 | 0.809 | 0.812 | 0.914 |
| 2012 | 0.021 | 0.524 | 0.549 | 0.641 | 0.698 | 0.743 | 0.962 |
| 2028 | 0.531 | 0.543 | 0.664 | 0.669 | 0.772 | 0.782 | 0.887 |
| 2041 | 0.104 | 0.111 | 0.221 | 0.427 | 0.469 | 0.469 | 0.935 |
| 2067 | 0.002 | 0.088 | 0.393 | 0.440 | 0.544 | 0.812 | 0.914 |
| 2101 | 0.140 | 0.362 | 0.521 | 0.531 | 0.700 | 0.822 | 0.826 |
| 2117 | 0.001 | 0.003 | 0.004 | 0.011 | 0.409 | 0.411 | 0.415 |
| 2142 | 0.095 | 0.125 | 0.519 | 0.543 | 0.586 | 0.592 | 0.799 |
| 2158 | 0.040 | 0.041 | 0.043 | 0.715 | 0.732 | 0.827 | 0.829 |
| 2174 | 0.627 | 0.654 | 0.757 | 0.759 | 0.859 | 0.874 | 0.934 |
| 2229 | 0.001 | 0.135 | 0.181 | 0.240 | 0.277 | 0.282 | 0.913 |
| 2304 | 0.015 | 0.016 | 0.061 | 0.107 | 0.822 | 0.878 | 0.889 |
| 2320 | 0.329 | 0.502 | 0.513 | 0.720 | 0.734 | 0.734 | 0.741 |
| 2391 | 0.001 | 0.004 | 0.004 | 0.744 | 0.760 | 0.771 | 0.781 |
| 2393 | 0.069 | 0.082 | 0.082 | 0.102 | 0.211 | 0.212 | 0.275 |
| 2466 | 0.003 | 0.008 | 0.013 | 0.744 | 0.746 | 0.817 | 0.818 |

TABLE 30
Correlation matrix for neutral glycans derived from embryonic stem cells.

| | 730 | 771 | 892 | 917 | 933 | 1054 | 1079 | 1095 | 1120 | 1136 | 1216 | 1241 | 1257 | 1282 | 1298 | 1323 | 1339 | 1378 |
|---|---|---|---|---|---|---|---|---|---|---|---|---|---|---|---|---|---|---|
| 730 | 1.00 | 0.69 | 0.68 | 0.53 | 0.22 | 0.54 | 0.41 | 0.14 | 0.40 | 0.27 | 0.35 | 0.15 | −0.09 | 0.33 | −0.28 | 0.17 | 0.30 | 0.08 |
| 771 | 0.69 | 1.00 | 0.83 | 0.84 | 0.78 | 0.74 | 0.77 | 0.61 | 0.63 | 0.04 | 0.56 | 0.47 | 0.09 | 0.39 | −0.22 | 0.40 | 0.53 | 0.53 |
| 892 | 0.68 | 0.83 | 1.00 | 0.58 | 0.64 | 0.84 | 0.60 | 0.58 | 0.46 | 0.29 | 0.75 | 0.24 | −0.01 | 0.34 | −0.08 | 0.20 | 0.37 | 0.58 |
| 917 | 0.53 | 0.84 | 0.58 | 1.00 | 0.74 | 0.59 | 0.95 | 0.50 | 0.72 | −0.31 | 0.33 | 0.70 | 0.30 | 0.56 | −0.47 | 0.57 | 0.70 | 0.36 |
| 933 | 0.22 | 0.78 | 0.64 | 0.74 | 1.00 | 0.62 | 0.79 | 0.87 | 0.54 | −0.15 | 0.63 | 0.61 | 0.23 | 0.29 | −0.10 | 0.33 | 0.46 | 0.73 |
| 1054 | 0.54 | 0.74 | 0.84 | 0.59 | 0.62 | 1.00 | 0.61 | 0.50 | 0.48 | 0.18 | 0.70 | 0.35 | 0.08 | 0.38 | −0.08 | 0.33 | 0.38 | 0.59 |
| 1079 | 0.41 | 0.77 | 0.60 | 0.95 | 0.79 | 0.61 | 1.00 | 0.59 | 0.65 | −0.27 | 0.35 | 0.72 | 0.42 | 0.60 | −0.35 | 0.48 | 0.64 | 0.37 |
| 1095 | 0.14 | 0.61 | 0.58 | 0.50 | 0.87 | 0.50 | 0.59 | 1.00 | 0.41 | 0.06 | 0.73 | 0.61 | 0.32 | 0.19 | 0.07 | 0.14 | 0.18 | 0.74 |
| 1120 | 0.40 | 0.63 | 0.46 | 0.72 | 0.54 | 0.48 | 0.65 | 0.41 | 1.00 | 0.04 | 0.31 | 0.74 | −0.10 | 0.87 | −0.71 | 0.82 | 0.83 | 0.46 |
| 1136 | 0.27 | 0.04 | 0.29 | −0.31 | −0.15 | 0.18 | −0.27 | 0.06 | 0.04 | 1.00 | 0.20 | −0.16 | −0.38 | 0.11 | 0.28 | −0.12 | −0.27 | 0.08 |
| 1216 | 0.35 | 0.56 | 0.75 | 0.33 | 0.63 | 0.70 | 0.35 | 0.73 | 0.31 | 0.20 | 1.00 | 0.26 | 0.03 | 0.08 | 0.01 | 0.07 | 0.04 | 0.88 |
| 1241 | 0.15 | 0.47 | 0.24 | 0.70 | 0.61 | 0.35 | 0.72 | 0.61 | 0.74 | −0.16 | 0.26 | 1.00 | 0.22 | 0.68 | −0.44 | 0.52 | 0.48 | 0.38 |
| 1257 | −0.09 | 0.09 | −0.01 | 0.30 | 0.23 | 0.08 | 0.42 | 0.32 | −0.10 | −0.38 | 0.03 | 0.22 | 1.00 | −0.09 | 0.25 | −0.11 | 0.01 | −0.03 |
| 1282 | 0.33 | 0.39 | 0.34 | 0.56 | 0.29 | 0.38 | 0.60 | 0.19 | 0.87 | 0.11 | 0.08 | 0.68 | −0.09 | 1.00 | −0.69 | 0.71 | 0.71 | 0.17 |
| 1298 | −0.28 | −0.22 | −0.08 | −0.47 | −0.10 | −0.08 | −0.35 | 0.07 | −0.71 | 0.28 | 0.01 | −0.44 | 0.25 | −0.69 | 1.00 | −0.70 | −0.73 | −0.05 |
| 1323 | 0.17 | 0.40 | 0.20 | 0.57 | 0.33 | 0.33 | 0.48 | 0.14 | 0.82 | −0.12 | 0.07 | 0.52 | −0.11 | 0.71 | −0.70 | 1.00 | 0.76 | 0.29 |
| 1339 | 0.30 | 0.53 | 0.37 | 0.70 | 0.46 | 0.38 | 0.64 | 0.18 | 0.83 | −0.27 | 0.04 | 0.48 | 0.01 | 0.71 | −0.73 | 0.76 | 1.00 | 0.20 |
| 1378 | 0.08 | 0.53 | 0.58 | 0.36 | 0.73 | 0.59 | 0.37 | 0.74 | 0.46 | 0.08 | 0.88 | 0.38 | −0.03 | 0.17 | −0.05 | 0.29 | 0.20 | 1.00 |
| 1393 | −0.10 | −0.36 | −0.43 | −0.22 | −0.26 | −0.57 | −0.24 | −0.10 | −0.25 | −0.25 | −0.16 | −0.01 | 0.24 | −0.29 | 0.08 | −0.48 | −0.31 | −0.20 |
| 1403 | 0.20 | 0.58 | 0.29 | 0.82 | 0.65 | 0.32 | 0.84 | 0.52 | 0.82 | −0.29 | 0.15 | 0.84 | 0.32 | 0.76 | −0.57 | 0.73 | 0.73 | 0.32 |
| 1419 | −0.50 | −0.31 | −0.52 | −0.01 | −0.02 | −0.42 | −0.05 | 0.08 | −0.22 | −0.67 | −0.21 | 0.25 | 0.22 | −0.28 | −0.12 | −0.08 | −0.14 | −0.15 |
| 1444 | −0.35 | −0.07 | −0.24 | 0.14 | 0.09 | −0.05 | 0.17 | 0.04 | 0.50 | −0.06 | −0.16 | 0.58 | −0.03 | 0.51 | −0.32 | 0.54 | 0.31 | 0.16 |
| 1460 | −0.29 | −0.22 | −0.02 | −0.38 | −0.12 | −0.05 | −0.19 | −0.10 | −0.53 | 0.16 | −0.09 | −0.54 | 0.37 | −0.39 | 0.75 | −0.47 | −0.40 | −0.08 |
| 1485 | 0.05 | −0.18 | 0.08 | −0.16 | −0.29 | −0.05 | 0.04 | −0.21 | −0.42 | 0.18 | −0.18 | −0.28 | 0.37 | −0.03 | 0.39 | −0.41 | −0.36 | −0.42 |
| 1501 | 0.41 | 0.63 | 0.45 | 0.75 | 0.53 | 0.44 | 0.69 | 0.20 | 0.83 | −0.21 | 0.13 | 0.47 | −0.07 | 0.72 | −0.75 | 0.79 | 0.95 | 0.26 |
| 1540 | 0.09 | 0.40 | 0.57 | 0.06 | 0.45 | 0.53 | 0.06 | 0.54 | 0.19 | 0.29 | 0.84 | −0.02 | −0.05 | −0.09 | 0.17 | 0.05 | −0.02 | 0.86 |
| 1555 | 0.16 | 0.06 | 0.04 | 0.08 | 0.13 | −0.22 | 0.09 | 0.08 | −0.24 | −0.22 | 0.12 | 0.00 | −0.04 | −0.28 | −0.02 | −0.39 | −0.23 | −0.06 |
| 1565 | 0.66 | 0.53 | 0.41 | 0.65 | 0.20 | 0.32 | 0.59 | 0.18 | 0.28 | −0.17 | 0.24 | 0.35 | 0.43 | 0.30 | −0.29 | 0.14 | 0.17 | −0.01 |
| 1581 | −0.26 | −0.40 | −0.61 | −0.19 | −0.44 | −0.60 | −0.34 | −0.36 | −0.19 | −0.41 | −0.51 | 0.06 | −0.17 | −0.18 | −0.28 | −0.06 | −0.11 | −0.51 |
| 1590 | 0.43 | 0.07 | 0.19 | −0.02 | −0.02 | 0.21 | −0.08 | 0.18 | 0.07 | 0.33 | 0.45 | 0.14 | −0.06 | −0.03 | −0.01 | −0.21 | −0.22 | 0.20 |
| 1606 | −0.16 | −0.24 | −0.09 | −0.12 | −0.05 | −0.11 | 0.15 | 0.10 | −0.31 | 0.12 | −0.20 | 0.07 | 0.48 | 0.07 | 0.41 | −0.39 | −0.34 | −0.32 |
| 1622 | −0.27 | −0.18 | −0.05 | −0.31 | −0.07 | −0.08 | −0.14 | −0.07 | −0.44 | 0.11 | −0.08 | −0.49 | 0.42 | −0.34 | 0.66 | −0.38 | −0.33 | −0.02 |
| 1647 | −0.13 | −0.37 | −0.34 | −0.21 | −0.28 | −0.30 | −0.04 | −0.14 | −0.43 | 0.13 | −0.39 | 0.00 | 0.44 | −0.10 | 0.48 | −0.51 | −0.51 | −0.50 |
| 1663 | −0.54 | −0.76 | −0.62 | −0.72 | −0.63 | −0.46 | −0.62 | −0.50 | −0.45 | 0.36 | −0.55 | −0.44 | −0.04 | −0.21 | 0.42 | −0.28 | −0.42 | −0.45 |
| 1688 | 0.46 | 0.18 | 0.24 | 0.36 | −0.12 | 0.12 | 0.39 | −0.02 | 0.14 | 0.05 | −0.07 | 0.22 | 0.40 | 0.36 | −0.15 | −0.04 | 0.10 | −0.33 |
| 1702 | 0.22 | 0.54 | 0.69 | 0.24 | 0.57 | 0.65 | 0.26 | 0.65 | 0.35 | 0.38 | 0.89 | 0.20 | −0.05 | 0.12 | 0.11 | 0.12 | 0.05 | 0.91 |
| 1704 | −0.28 | −0.14 | −0.06 | −0.05 | 0.12 | −0.06 | 0.21 | 0.04 | −0.22 | 0.01 | −0.22 | −0.04 | 0.34 | 0.08 | 0.41 | −0.19 | −0.15 | −0.13 |
| 1717 | 0.29 | 0.16 | 0.18 | 0.02 | 0.12 | −0.16 | −0.01 | 0.37 | −0.04 | 0.26 | 0.26 | 0.04 | −0.10 | −0.08 | 0.01 | −0.22 | −0.24 | 0.06 |
| 1743 | −0.19 | −0.62 | −0.62 | −0.62 | −0.85 | −0.65 | −0.78 | −0.82 | −0.48 | −0.05 | −0.65 | −0.59 | −0.45 | −0.39 | −0.05 | −0.22 | −0.30 | −0.69 |
| 1768 | 0.25 | 0.07 | 0.02 | 0.05 | −0.05 | 0.01 | 0.12 | −0.06 | 0.04 | 0.25 | −0.19 | −0.07 | 0.24 | 0.20 | −0.08 | 0.15 | 0.08 | −0.30 |
| 1793 | 0.21 | 0.44 | 0.21 | 0.51 | 0.48 | 0.25 | 0.39 | 0.25 | 0.79 | −0.03 | 0.24 | 0.44 | −0.30 | 0.53 | −0.64 | 0.77 | 0.69 | 0.49 |
| 1809 | −0.60 | −0.79 | −0.84 | −0.56 | −0.62 | −0.68 | −0.54 | −0.62 | −0.45 | −0.17 | −0.67 | −0.26 | −0.02 | −0.30 | 0.23 | −0.34 | −0.38 | −0.51 |
| 1825 | 0.54 | 0.37 | 0.38 | 0.44 | 0.01 | 0.21 | 0.40 | −0.24 | 0.36 | −0.17 | −0.09 | −0.07 | 0.04 | 0.46 | −0.57 | 0.41 | 0.61 | −0.21 |
| 1850 | 0.24 | 0.11 | 0.16 | 0.20 | 0.06 | 0.08 | 0.37 | 0.05 | 0.06 | 0.16 | −0.06 | 0.04 | 0.51 | 0.33 | 0.10 | −0.02 | 0.03 | −0.15 |
| 1866 | −0.16 | −0.08 | 0.06 | −0.05 | 0.08 | 0.00 | 0.17 | −0.07 | −0.22 | −0.07 | −0.13 | −0.28 | 0.31 | 0.03 | 0.29 | −0.11 | −0.03 | −0.08 |
| 1905 | −0.24 | −0.51 | −0.31 | −0.73 | −0.55 | −0.38 | −0.78 | −0.56 | −0.39 | 0.29 | −0.26 | −0.73 | −0.74 | −0.35 | 0.07 | −0.16 | −0.27 | −0.23 |
| 1955 | −0.14 | −0.63 | −0.73 | −0.51 | −0.76 | −0.66 | −0.67 | −0.73 | −0.37 | −0.01 | −0.58 | −0.35 | −0.30 | −0.34 | −0.02 | −0.25 | −0.36 | −0.57 |
| 1996 | 0.32 | 0.46 | 0.26 | 0.69 | 0.36 | 0.25 | 0.61 | 0.18 | 0.86 | −0.20 | 0.12 | 0.57 | 0.07 | 0.77 | −0.83 | 0.82 | 0.83 | 0.25 |
| 2012 | −0.09 | −0.10 | 0.07 | −0.15 | 0.19 | 0.08 | 0.12 | 0.31 | −0.24 | 0.33 | 0.12 | 0.06 | 0.33 | −0.02 | 0.56 | −0.44 | −0.40 | 0.05 |
| 2028 | −0.46 | −0.79 | −0.74 | −0.69 | −0.61 | −0.63 | −0.66 | −0.43 | −0.61 | 0.00 | −0.54 | −0.25 | −0.14 | −0.45 | 0.30 | −0.50 | −0.59 | −0.57 |
| 2041 | −0.15 | −0.47 | −0.24 | −0.57 | −0.29 | −0.33 | −0.42 | −0.05 | −0.47 | 0.41 | −0.13 | −0.13 | −0.25 | −0.24 | 0.40 | −0.64 | −0.62 | −0.30 |
| 2067 | −0.46 | −0.46 | −0.26 | −0.68 | −0.28 | −0.29 | −0.66 | −0.21 | −0.57 | 0.10 | −0.07 | −0.57 | −0.53 | −0.54 | 0.30 | −0.31 | −0.47 | −0.06 |
| 2101 | −0.29 | −0.16 | −0.46 | 0.07 | −0.02 | −0.30 | −0.05 | −0.03 | 0.30 | −0.14 | −0.27 | 0.47 | −0.18 | 0.13 | −0.24 | 0.29 | 0.14 | 0.00 |
| 2142 | −0.30 | −0.02 | 0.01 | −0.33 | −0.06 | 0.01 | −0.39 | 0.09 | −0.35 | 0.03 | 0.17 | −0.40 | 0.06 | −0.56 | 0.48 | −0.25 | −0.29 | 0.23 |
| 2158 | 0.47 | 0.19 | 0.31 | 0.02 | 0.13 | 0.16 | 0.05 | 0.44 | 0.16 | 0.54 | 0.44 | 0.21 | 0.15 | 0.07 | 0.13 | −0.18 | −0.18 | 0.22 |
| 2174 | −0.47 | −0.81 | −0.79 | −0.66 | −0.67 | −0.65 | −0.67 | −0.54 | −0.59 | −0.08 | −0.59 | −0.28 | −0.16 | −0.44 | 0.24 | −0.51 | −0.54 | −0.59 |
| 2229 | −0.14 | −0.24 | 0.04 | −0.32 | 0.00 | −0.04 | −0.09 | 0.08 | −0.36 | 0.34 | 0.01 | −0.10 | −0.16 | −0.08 | 0.41 | −0.59 | −0.41 | −0.11 |
| 2304 | 0.72 | 0.54 | 0.51 | 0.60 | 0.16 | 0.36 | 0.53 | 0.19 | 0.31 | 0.08 | 0.23 | 0.29 | 0.30 | 0.32 | −0.32 | 0.20 | 0.25 | −0.08 |

| | 1393 | 1403 | 1419 | 1444 | 1460 | 1485 | 1501 | 1540 | 1555 | 1565 | 1581 | 1590 | 1606 | 1622 | 1647 | 1663 | 1688 | 1702 | 1704 | 1717 |
|---|---|---|---|---|---|---|---|---|---|---|---|---|---|---|---|---|---|---|---|---|
| 730 | −0.10 | 0.20 | −0.50 | −0.35 | −0.29 | 0.05 | 0.41 | 0.09 | 0.16 | 0.66 | −0.26 | 0.43 | −0.16 | −0.27 | −0.13 | −0.54 | 0.46 | 0.22 | −0.28 | 0.29 |
| 771 | −0.36 | 0.58 | −0.31 | −0.07 | −0.22 | −0.18 | 0.63 | 0.40 | 0.06 | 0.53 | −0.40 | 0.07 | −0.24 | −0.18 | −0.37 | −0.76 | 0.18 | 0.54 | −0.14 | 0.16 |
| 892 | −0.43 | 0.29 | −0.52 | −0.24 | −0.02 | 0.08 | 0.45 | 0.57 | 0.04 | 0.41 | −0.61 | 0.19 | −0.09 | −0.05 | −0.34 | −0.62 | 0.24 | 0.69 | −0.06 | 0.18 |
| 917 | −0.22 | 0.82 | −0.01 | 0.14 | −0.38 | −0.16 | 0.75 | 0.06 | 0.08 | 0.65 | −0.19 | −0.02 | −0.12 | −0.31 | −0.21 | −0.72 | 0.36 | 0.24 | −0.05 | 0.02 |
| 933 | −0.26 | 0.65 | −0.02 | 0.09 | −0.12 | −0.29 | 0.53 | 0.45 | 0.13 | 0.20 | −0.44 | −0.02 | −0.05 | −0.07 | −0.28 | −0.63 | −0.12 | 0.57 | 0.12 | 0.12 |
| 1054 | −0.57 | 0.32 | −0.42 | −0.05 | −0.05 | −0.05 | 0.44 | 0.53 | −0.22 | 0.32 | −0.60 | 0.21 | −0.11 | −0.08 | −0.30 | −0.46 | 0.12 | 0.65 | −0.06 | −0.16 |
| 1079 | −0.24 | 0.84 | −0.05 | 0.17 | −0.19 | 0.04 | 0.69 | 0.06 | 0.09 | 0.59 | −0.34 | −0.08 | 0.15 | −0.14 | −0.04 | −0.62 | 0.39 | 0.26 | 0.21 | −0.01 |
| 1095 | −0.10 | 0.52 | 0.08 | 0.04 | −0.10 | −0.21 | 0.20 | 0.54 | 0.08 | 0.18 | −0.36 | 0.18 | 0.10 | −0.07 | −0.14 | −0.50 | −0.02 | 0.65 | 0.04 | 0.37 |
| 1120 | −0.25 | 0.82 | −0.22 | 0.50 | −0.53 | −0.42 | 0.83 | 0.19 | −0.24 | 0.28 | −0.19 | 0.07 | −0.31 | −0.44 | −0.43 | −0.45 | 0.14 | 0.35 | −0.22 | −0.04 |
| 1136 | −0.25 | −0.29 | −0.67 | −0.06 | 0.16 | 0.18 | −0.21 | 0.29 | −0.22 | −0.17 | −0.41 | 0.33 | 0.12 | 0.11 | 0.13 | 0.36 | 0.05 | 0.38 | 0.01 | 0.26 |
| 1216 | −0.16 | 0.15 | −0.21 | −0.16 | −0.09 | −0.18 | 0.13 | 0.84 | 0.12 | 0.24 | −0.51 | 0.45 | −0.20 | −0.08 | −0.39 | −0.55 | −0.07 | 0.89 | −0.22 | 0.26 |
| 1241 | −0.01 | 0.84 | 0.25 | 0.58 | −0.54 | −0.28 | 0.47 | −0.02 | 0.00 | 0.35 | 0.06 | 0.14 | 0.07 | −0.49 | 0.00 | −0.44 | 0.22 | 0.20 | −0.04 | 0.04 |
| 1257 | 0.24 | 0.32 | 0.22 | −0.03 | 0.37 | 0.37 | −0.07 | −0.05 | −0.04 | 0.43 | −0.17 | −0.06 | 0.48 | 0.42 | 0.44 | −0.04 | 0.40 | −0.05 | 0.34 | −0.10 |
| 1282 | −0.29 | 0.76 | −0.28 | 0.51 | −0.39 | −0.03 | 0.72 | −0.09 | −0.28 | 0.30 | −0.18 | −0.03 | 0.07 | −0.34 | −0.10 | −0.21 | 0.36 | 0.12 | 0.08 | −0.08 |
| 1298 | 0.08 | −0.57 | −0.12 | −0.32 | 0.75 | 0.39 | −0.75 | 0.17 | −0.02 | −0.29 | −0.28 | −0.01 | 0.41 | 0.66 | 0.48 | 0.42 | −0.15 | 0.11 | 0.41 | 0.01 |
| 1323 | −0.48 | 0.73 | −0.08 | 0.54 | −0.47 | −0.41 | 0.79 | 0.05 | −0.39 | 0.14 | −0.06 | −0.21 | −0.39 | −0.38 | −0.51 | −0.28 | −0.04 | 0.12 | −0.19 | −0.22 |
| 1339 | −0.31 | 0.73 | −0.14 | 0.31 | −0.40 | −0.36 | 0.95 | −0.02 | −0.23 | 0.17 | −0.11 | −0.22 | −0.34 | −0.33 | −0.51 | −0.42 | 0.10 | 0.05 | −0.15 | −0.24 |
| 1378 | −0.20 | 0.32 | −0.15 | 0.16 | −0.08 | −0.42 | 0.26 | 0.86 | −0.06 | −0.01 | −0.51 | 0.20 | −0.32 | −0.02 | −0.50 | −0.45 | −0.33 | 0.91 | −0.13 | 0.06 |
| 1393 | 1.00 | −0.18 | 0.30 | −0.02 | −0.04 | 0.01 | −0.39 | −0.23 | 0.42 | 0.12 | 0.31 | 0.33 | 0.12 | 0.04 | 0.39 | 0.05 | 0.18 | −0.25 | −0.07 | 0.17 |
| 1403 | −0.18 | 1.00 | 0.12 | 0.44 | −0.39 | −0.20 | 0.73 | −0.05 | −0.13 | 0.44 | −0.08 | −0.17 | 0.05 | −0.28 | −0.13 | −0.46 | 0.27 | 0.12 | 0.09 | 0.05 |
| 1419 | 0.30 | 0.12 | 1.00 | 0.10 | −0.40 | −0.28 | −0.22 | −0.33 | 0.28 | 0.00 | 0.78 | −0.09 | −0.03 | −0.41 | 0.02 | −0.16 | −0.10 | −0.40 | −0.23 | 0.15 |
| 1444 | −0.02 | 0.44 | 0.10 | 1.00 | −0.22 | −0.25 | 0.29 | −0.04 | −0.24 | −0.16 | 0.09 | −0.20 | −0.07 | −0.18 | −0.06 | 0.08 | −0.17 | 0.03 | 0.06 | −0.53 |
| 1460 | −0.04 | −0.39 | −0.40 | −0.22 | 1.00 | 0.62 | −0.42 | 0.16 | −0.22 | −0.24 | −0.58 | −0.35 | 0.52 | 0.97 | 0.42 | 0.49 | −0.04 | 0.08 | 0.70 | −0.26 |
| 1485 | 0.01 | −0.20 | −0.28 | −0.25 | 0.62 | 1.00 | −0.33 | −0.27 | 0.07 | 0.32 | −0.29 | −0.19 | 0.80 | 0.55 | 0.69 | 0.27 | 0.60 | −0.20 | 0.65 | 0.02 |
| 1501 | −0.39 | 0.73 | −0.22 | 0.29 | −0.42 | −0.33 | 1.00 | 0.03 | −0.07 | 0.24 | −0.20 | −0.19 | −0.35 | −0.34 | −0.53 | −0.52 | 0.04 | 0.11 | −0.12 | −0.20 |
| 1540 | −0.23 | −0.05 | −0.33 | −0.04 | 0.16 | −0.27 | 0.03 | 1.00 | −0.10 | −0.08 | −0.55 | 0.25 | −0.36 | 0.18 | −0.53 | −0.28 | −0.36 | 0.94 | −0.23 | 0.02 |
| 1555 | 0.42 | −0.13 | 0.28 | −0.24 | −0.22 | 0.07 | −0.07 | −0.10 | 1.00 | 0.25 | 0.23 | 0.27 | 0.03 | −0.24 | 0.07 | −0.47 | 0.00 | −0.11 | −0.05 | 0.33 |
| 1565 | 0.12 | 0.44 | 0.00 | −0.16 | −0.24 | 0.32 | 0.24 | −0.08 | 0.25 | 1.00 | −0.01 | 0.21 | 0.14 | −0.18 | 0.17 | −0.58 | 0.76 | 0.07 | −0.11 | 0.26 |
| 1581 | 0.31 | −0.08 | 0.78 | 0.09 | −0.58 | −0.29 | −0.20 | −0.55 | 0.23 | −0.01 | 1.00 | −0.02 | −0.22 | −0.61 | −0.04 | −0.04 | 0.00 | −0.63 | −0.48 | 0.15 |
| 1590 | 0.33 | −0.17 | −0.09 | −0.20 | −0.35 | −0.19 | −0.19 | 0.25 | 0.27 | 0.21 | −0.02 | 1.00 | −0.14 | −0.36 | 0.03 | −0.11 | 0.11 | 0.31 | −0.44 | 0.31 |
| 1606 | 0.12 | 0.05 | −0.03 | −0.07 | 0.52 | 0.80 | −0.35 | −0.36 | 0.03 | 0.14 | −0.22 | −0.14 | 1.00 | 0.47 | 0.87 | 0.36 | 0.45 | −0.24 | 0.81 | 0.13 |
| 1622 | 0.04 | −0.28 | −0.41 | −0.18 | 0.97 | 0.55 | −0.34 | 0.18 | −0.24 | −0.18 | −0.61 | −0.36 | 0.47 | 1.00 | 0.41 | 0.47 | −0.05 | 0.11 | 0.70 | −0.24 |
| 1647 | 0.39 | −0.13 | 0.02 | −0.06 | 0.42 | 0.69 | −0.53 | −0.53 | 0.07 | 0.17 | −0.04 | 0.03 | 0.87 | 0.41 | 1.00 | 0.49 | 0.48 | −0.40 | 0.64 | 0.09 |
| 1663 | 0.05 | −0.46 | −0.16 | 0.08 | 0.49 | 0.27 | −0.52 | −0.28 | −0.47 | −0.58 | −0.04 | −0.11 | 0.36 | 0.47 | 0.49 | 1.00 | −0.12 | −0.33 | 0.38 | −0.21 |
| 1688 | 0.18 | 0.27 | −0.10 | −0.17 | −0.04 | 0.60 | 0.04 | −0.36 | 0.00 | 0.76 | 0.00 | 0.11 | 0.45 | −0.05 | 0.48 | −0.12 | 1.00 | −0.17 | 0.10 | 0.25 |
| 1702 | −0.25 | 0.12 | −0.40 | 0.03 | 0.08 | −0.20 | 0.11 | 0.94 | −0.11 | 0.07 | −0.63 | 0.31 | −0.24 | 0.11 | −0.40 | −0.33 | −0.17 | 1.00 | −0.14 | 0.10 |
| 1704 | −0.07 | 0.09 | −0.23 | 0.06 | 0.70 | 0.65 | −0.12 | −0.23 | −0.05 | −0.11 | −0.48 | −0.44 | 0.81 | 0.70 | 0.64 | 0.38 | 0.10 | −0.14 | 1.00 | −0.16 |
| 1717 | 0.17 | 0.05 | 0.15 | −0.53 | −0.26 | 0.02 | −0.20 | 0.02 | 0.33 | 0.26 | 0.15 | 0.31 | 0.13 | −0.24 | 0.09 | −0.21 | 0.25 | 0.10 | −0.16 | 1.00 |
| 1743 | 0.16 | −0.59 | 0.23 | −0.16 | −0.16 | −0.08 | −0.35 | −0.46 | 0.03 | −0.29 | 0.69 | −0.08 | −0.29 | −0.21 | −0.07 | 0.35 | −0.15 | −0.64 | −0.38 | −0.03 |
| 1768 | −0.24 | 0.17 | −0.31 | −0.03 | 0.17 | 0.34 | 0.21 | −0.17 | −0.01 | 0.22 | −0.22 | −0.07 | 0.34 | 0.23 | 0.23 | 0.14 | 0.12 | −0.18 | 0.28 | 0.05 |
| 1793 | −0.20 | 0.57 | −0.15 | 0.38 | −0.52 | −0.69 | 0.76 | 0.23 | −0.09 | −0.06 | −0.16 | 0.08 | −0.58 | −0.39 | −0.59 | −0.31 | −0.30 | 0.30 | −0.28 | −0.05 |
| 1809 | 0.54 | −0.42 | 0.23 | 0.30 | 0.21 | 0.08 | −0.48 | −0.50 | −0.03 | −0.43 | 0.34 | −0.12 | 0.16 | 0.21 | 0.47 | 0.66 | −0.12 | −0.55 | 0.22 | −0.40 |
| 1825 | −0.27 | 0.33 | −0.35 | −0.16 | −0.08 | 0.27 | 0.66 | −0.18 | 0.00 | 0.50 | −0.14 | −0.27 | −0.08 | −0.04 | −0.24 | −0.36 | 0.44 | −0.17 | 0.00 | −0.07 |
| 1850 | 0.07 | 0.28 | −0.45 | −0.08 | 0.50 | 0.71 | 0.07 | −0.17 | −0.10 | 0.43 | −0.55 | −0.11 | 0.72 | 0.58 | 0.62 | 0.19 | 0.57 | −0.03 | 0.70 | 0.07 |
| 1866 | −0.15 | 0.03 | −0.37 | −0.13 | 0.77 | 0.65 | 0.02 | −0.08 | −0.08 | −0.06 | −0.60 | −0.51 | 0.62 | 0.79 | 0.39 | 0.28 | 0.07 | −0.08 | 0.89 | −0.19 |
| 1905 | −0.16 | −0.68 | −0.19 | −0.21 | 0.09 | −0.15 | −0.23 | 0.00 | −0.06 | −0.69 | 0.13 | −0.14 | −0.32 | 0.04 | −0.29 | 0.43 | −0.55 | −0.18 | −0.15 | −0.02 |
| 1955 | 0.55 | −0.51 | 0.19 | 0.05 | −0.21 | −0.17 | −0.40 | −0.47 | 0.13 | −0.20 | 0.57 | 0.26 | −0.25 | −0.20 | 0.19 | 0.42 | −0.08 | −0.55 | −0.34 | −0.10 |
| 1996 | −0.10 | 0.79 | −0.10 | 0.44 | −0.48 | −0.28 | 0.84 | 0.00 | −0.16 | 0.41 | −0.08 | −0.12 | −0.29 | −0.34 | −0.37 | −0.43 | 0.27 | 0.11 | −0.19 | −0.10 |
| 2012 | 0.14 | −0.05 | −0.27 | −0.09 | 0.57 | 0.55 | −0.36 | 0.00 | 0.08 | −0.07 | −0.53 | 0.13 | 0.83 | 0.55 | 0.73 | 0.35 | 0.12 | 0.13 | 0.78 | 0.14 |
| 2028 | 0.46 | −0.56 | 0.41 | 0.05 | 0.00 | 0.06 | −0.65 | −0.53 | 0.16 | −0.43 | 0.54 | 0.07 | 0.24 | −0.07 | 0.47 | 0.52 | −0.16 | −0.61 | 0.02 | −0.04 |
| 2041 | 0.29 | −0.47 | 0.06 | −0.15 | 0.08 | 0.28 | −0.59 | −0.26 | 0.37 | −0.32 | 0.15 | 0.33 | 0.50 | −0.03 | 0.54 | 0.37 | −0.04 | −0.22 | 0.24 | 0.33 |
| 2067 | −0.26 | −0.61 | 0.20 | −0.28 | 0.13 | −0.12 | −0.43 | 0.10 | 0.08 | −0.67 | 0.23 | −0.20 | −0.13 | 0.02 | −0.24 | 0.26 | −0.64 | −0.09 | −0.05 | 0.13 |
| 2101 | 0.32 | 0.22 | 0.40 | 0.63 | −0.53 | −0.60 | 0.06 | −0.24 | −0.04 | −0.24 | 0.47 | 0.13 | −0.34 | −0.48 | −0.05 | 0.09 | −0.19 | −0.18 | −0.31 | −0.17 |
| 2142 | −0.19 | −0.37 | 0.07 | −0.17 | 0.35 | −0.15 | −0.37 | 0.55 | −0.27 | −0.27 | 0.01 | −0.18 | −0.30 | 0.31 | −0.34 | 0.05 | −0.39 | 0.34 | −0.25 | −0.07 |
| 2158 | 0.36 | 0.02 | −0.28 | −0.18 | −0.10 | 0.02 | −0.16 | 0.26 | 0.14 | 0.26 | −0.26 | 0.77 | 0.16 | −0.06 | 0.23 | −0.05 | 0.27 | 0.36 | −0.17 | 0.51 |
| 2174 | 0.53 | −0.57 | 0.42 | 0.04 | −0.03 | 0.01 | −0.63 | −0.55 | 0.11 | −0.41 | 0.58 | 0.08 | 0.15 | −0.09 | 0.44 | 0.54 | −0.11 | −0.62 | −0.04 | −0.10 |
| 2229 | 0.06 | −0.31 | −0.15 | −0.22 | 0.32 | 0.46 | −0.38 | −0.15 | 0.27 | −0.28 | −0.23 | 0.07 | 0.65 | 0.21 | 0.54 | 0.31 | 0.05 | −0.03 | 0.57 | 0.21 |
| 2304 | −0.06 | 0.37 | −0.09 | −0.23 | −0.33 | 0.28 | 0.31 | −0.08 | 0.21 | 0.87 | −0.01 | 0.27 | 0.06 | −0.32 | 0.05 | −0.52 | 0.77 | 0.06 | −0.25 | 0.36 |
| | 1743 | 1768 | 1793 | 1809 | 1825 | 1850 | 1866 | 1905 | 1955 | 1996 | 2012 | 2028 | 2041 | 2067 | 2101 | 2142 | 2158 | 2174 | 2229 | 2304 |
| 730 | −0.19 | 0.25 | 0.21 | −0.60 | 0.54 | 0.24 | −0.16 | −0.24 | −0.14 | 0.32 | −0.09 | −0.46 | −0.15 | −0.46 | −0.29 | −0.30 | 0.47 | −0.47 | −0.14 | 0.72 |
| 771 | −0.62 | 0.07 | 0.44 | −0.79 | 0.37 | 0.11 | −0.08 | −0.51 | −0.63 | 0.46 | −0.10 | −0.79 | −0.47 | −0.46 | −0.16 | −0.02 | 0.19 | −0.81 | −0.24 | 0.54 |
| 892 | −0.62 | 0.02 | 0.21 | −0.84 | 0.38 | 0.16 | 0.06 | −0.31 | −0.73 | 0.26 | 0.07 | −0.74 | −0.24 | −0.26 | −0.46 | 0.01 | 0.31 | −0.79 | 0.04 | 0.51 |
| 917 | −0.62 | 0.05 | 0.51 | −0.56 | 0.44 | 0.20 | −0.05 | −0.73 | −0.51 | 0.69 | −0.15 | −0.69 | −0.57 | −0.68 | 0.07 | −0.33 | 0.02 | −0.66 | −0.32 | 0.60 |
| 933 | −0.85 | −0.05 | 0.48 | −0.62 | 0.01 | 0.06 | 0.08 | −0.55 | −0.76 | 0.36 | 0.19 | −0.61 | −0.29 | −0.28 | −0.02 | −0.06 | 0.13 | −0.67 | 0.00 | 0.16 |
| 1054 | −0.65 | 0.01 | 0.25 | −0.68 | 0.21 | 0.08 | 0.00 | −0.38 | −0.66 | 0.25 | 0.08 | −0.63 | −0.33 | −0.29 | −0.30 | 0.01 | 0.16 | −0.65 | −0.04 | 0.36 |
| 1079 | −0.78 | 0.12 | 0.39 | −0.54 | 0.40 | 0.31 | 0.17 | −0.78 | −0.67 | 0.61 | 0.12 | −0.66 | −0.42 | −0.66 | −0.05 | −0.39 | 0.05 | −0.67 | −0.09 | 0.53 |
| 1095 | −0.82 | −0.06 | 0.25 | −0.62 | −0.24 | 0.05 | −0.07 | −0.56 | −0.73 | 0.18 | 0.31 | −0.43 | −0.05 | −0.21 | −0.03 | 0.09 | 0.44 | −0.54 | 0.08 | 0.19 |
| 1120 | −0.48 | 0.04 | 0.79 | −0.45 | 0.36 | 0.06 | −0.22 | −0.39 | −0.37 | 0.86 | −0.24 | −0.61 | −0.47 | −0.57 | 0.30 | −0.35 | 0.16 | −0.59 | −0.36 | 0.31 |
| 1136 | −0.05 | 0.25 | −0.03 | −0.17 | −0.17 | 0.16 | −0.07 | 0.29 | −0.01 | −0.20 | 0.33 | 0.00 | 0.41 | 0.10 | −0.14 | 0.03 | 0.54 | −0.08 | 0.34 | 0.08 |
| 1216 | −0.65 | −0.19 | 0.24 | −0.67 | −0.09 | −0.06 | −0.13 | −0.26 | −0.58 | 0.12 | 0.12 | −0.54 | −0.13 | −0.27 | −0.27 | 0.17 | 0.44 | −0.59 | 0.01 | 0.23 |
| 1241 | −0.59 | −0.07 | 0.44 | −0.26 | −0.07 | 0.04 | −0.28 | −0.73 | −0.35 | 0.57 | 0.06 | −0.25 | −0.13 | −0.57 | 0.47 | −0.40 | 0.21 | −0.28 | −0.10 | 0.29 |
| 1257 | −0.45 | 0.24 | −0.30 | −0.02 | 0.04 | 0.51 | 0.31 | −0.74 | −0.30 | 0.07 | 0.33 | −0.14 | −0.25 | −0.53 | −0.18 | 0.06 | 0.15 | −0.16 | −0.16 | 0.30 |
| 1282 | −0.39 | 0.20 | 0.53 | −0.30 | 0.46 | 0.33 | 0.03 | −0.35 | −0.34 | 0.77 | −0.02 | −0.45 | −0.24 | −0.54 | 0.13 | −0.56 | 0.07 | −0.44 | −0.08 | 0.32 |
| 1298 | −0.05 | −0.08 | −0.64 | 0.23 | −0.57 | 0.10 | 0.29 | 0.07 | −0.02 | −0.83 | 0.56 | 0.30 | 0.40 | 0.30 | −0.24 | 0.48 | 0.13 | 0.24 | 0.41 | −0.32 |
| 1323 | −0.22 | 0.15 | 0.77 | −0.34 | 0.41 | −0.02 | −0.11 | −0.16 | −0.25 | 0.82 | −0.44 | −0.50 | −0.64 | −0.31 | 0.29 | −0.25 | −0.18 | 0.51 | −0.59 | 0.20 |
| 1339 | −0.30 | 0.08 | 0.69 | −0.38 | 0.61 | 0.03 | −0.03 | −0.27 | −0.36 | 0.83 | −0.40 | −0.59 | −0.62 | −0.47 | 0.14 | −0.29 | −0.18 | −0.54 | −0.41 | 0.25 |
| 1378 | −0.69 | −0.30 | 0.49 | −0.51 | −0.21 | −0.15 | −0.08 | −0.23 | −0.57 | 0.25 | 0.05 | −0.57 | −0.30 | −0.06 | 0.00 | 0.23 | 0.22 | −0.59 | −0.11 | −0.08 |
| 1393 | 0.16 | −0.24 | −0.20 | 0.54 | −0.27 | 0.07 | −0.15 | −0.16 | 0.55 | −0.10 | 0.14 | 0.46 | 0.29 | −0.26 | 0.32 | −0.19 | 0.36 | 0.53 | 0.06 | −0.06 |
| 1403 | −0.59 | 0.17 | 0.57 | −0.42 | 0.33 | 0.28 | 0.03 | −0.68 | −0.51 | 0.79 | −0.05 | −0.56 | −0.47 | −0.61 | 0.22 | −0.37 | 0.02 | −0.57 | −0.31 | 0.37 |
| 1419 | 0.23 | −0.31 | −0.15 | 0.23 | −0.35 | −0.45 | −0.37 | −0.19 | 0.19 | −0.10 | −0.27 | 0.41 | 0.06 | 0.20 | 0.40 | 0.07 | −0.28 | 0.42 | −0.15 | −0.09 |
| 1444 | −0.16 | −0.03 | 0.38 | 0.30 | −0.16 | −0.08 | −0.13 | −0.21 | 0.05 | 0.44 | −0.09 | 0.05 | −0.15 | −0.28 | 0.63 | −0.17 | −0.18 | 0.04 | −0.22 | −0.23 |
| 1460 | −0.16 | 0.17 | −0.52 | 0.21 | −0.08 | 0.50 | 0.77 | 0.09 | −0.21 | −0.48 | 0.57 | 0.00 | 0.08 | 0.13 | −0.53 | 0.35 | −0.10 | −0.03 | 0.32 | −0.33 |
| 1485 | −0.08 | 0.34 | −0.69 | 0.08 | 0.27 | 0.71 | 0.65 | −0.15 | −0.17 | −0.28 | 0.55 | 0.06 | 0.28 | −0.12 | −0.60 | −0.15 | 0.02 | 0.01 | 0.46 | 0.28 |
| 1501 | −0.35 | 0.21 | 0.76 | −0.48 | 0.66 | 0.07 | 0.02 | −0.23 | −0.40 | 0.84 | −0.36 | −0.65 | −0.59 | −0.43 | 0.06 | −0.37 | −0.16 | −0.63 | −0.38 | 0.31 |
| 1540 | −0.46 | −0.17 | 0.23 | −0.50 | −0.18 | −0.17 | −0.08 | 0.20 | −0.47 | 0.00 | 0.00 | −0.53 | −0.26 | 0.10 | −0.24 | 0.55 | 0.26 | −0.55 | −0.15 | −0.08 |
| 1555 | 0.03 | −0.01 | −0.09 | −0.03 | 0.00 | −0.10 | −0.08 | −0.06 | 0.13 | −0.16 | 0.08 | 0.16 | 0.37 | 0.08 | −0.04 | −0.27 | 0.14 | 0.11 | 0.27 | 0.21 |
| 1565 | −0.29 | 0.22 | −0.06 | −0.43 | 0.50 | 0.43 | −0.06 | −0.69 | −0.20 | 0.41 | −0.07 | −0.43 | −0.32 | −0.67 | −0.24 | −0.27 | 0.26 | −0.41 | −0.28 | 0.87 |
| 1581 | 0.69 | −0.22 | −0.16 | 0.34 | −0.14 | −0.55 | −0.60 | 0.13 | 0.57 | −0.08 | −0.53 | 0.54 | 0.15 | 0.23 | 0.47 | 0.01 | −0.26 | 0.58 | −0.23 | −0.01 |
| 1590 | −0.08 | −0.07 | 0.08 | −0.12 | −0.27 | −0.11 | −0.51 | −0.14 | 0.26 | −0.12 | 0.13 | 0.07 | 0.33 | −0.20 | 0.13 | −0.18 | 0.77 | 0.08 | 0.07 | 0.27 |
| 1606 | −0.29 | 0.34 | −0.58 | 0.16 | −0.08 | 0.72 | 0.62 | −0.32 | −0.25 | −0.29 | 0.83 | 0.24 | 0.50 | −0.13 | −0.34 | −0.30 | 0.16 | 0.15 | 0.65 | 0.06 |
| 1622 | −0.21 | 0.23 | −0.39 | 0.21 | −0.04 | 0.58 | 0.79 | 0.04 | −0.20 | −0.34 | 0.55 | −0.07 | −0.03 | 0.02 | −0.48 | 0.31 | −0.06 | −0.09 | 0.21 | −0.32 |
| 1647 | −0.07 | 0.23 | −0.59 | 0.47 | −0.24 | 0.62 | 0.39 | −0.29 | 0.19 | −0.37 | 0.73 | 0.47 | 0.54 | −0.24 | −0.05 | −0.34 | 0.23 | 0.44 | 0.54 | 0.05 |
| 1663 | 0.35 | 0.14 | −0.31 | 0.66 | −0.36 | 0.19 | 0.28 | 0.43 | 0.42 | −0.43 | 0.35 | 0.52 | 0.37 | 0.26 | 0.09 | 0.25 | −0.05 | 0.54 | 0.31 | −0.52 |
| 1688 | −0.15 | 0.12 | −0.30 | −0.12 | 0.44 | 0.57 | 0.07 | −0.55 | −0.08 | 0.27 | 0.12 | −0.16 | −0.04 | −0.64 | −0.19 | −0.39 | 0.27 | −0.11 | 0.05 | 0.77 |
| 1702 | −0.64 | −0.18 | 0.30 | −0.55 | −0.17 | −0.03 | −0.08 | −0.18 | −0.55 | 0.11 | 0.13 | −0.61 | −0.22 | −0.09 | −0.18 | 0.34 | 0.36 | −0.62 | −0.03 | 0.06 |
| 1704 | −0.38 | 0.28 | −0.28 | 0.22 | 0.00 | 0.70 | 0.89 | −0.15 | −0.34 | −0.19 | 0.78 | 0.02 | 0.24 | −0.05 | −0.31 | −0.25 | −0.17 | −0.04 | 0.57 | −0.25 |
| 1717 | −0.03 | 0.05 | −0.05 | −0.40 | −0.07 | 0.07 | −0.19 | −0.02 | −0.10 | −0.10 | 0.14 | −0.04 | 0.33 | 0.13 | −0.17 | −0.07 | 0.51 | −0.10 | 0.21 | 0.36 |
| 1743 | 1.00 | −0.06 | −0.29 | 0.47 | 0.02 | −0.44 | −0.31 | 0.68 | 0.76 | −0.33 | −0.51 | 0.58 | 0.19 | 0.49 | 0.13 | 0.17 | −0.32 | 0.63 | −0.17 | −0.21 |
| 1768 | −0.06 | 1.00 | −0.02 | −0.25 | 0.41 | 0.56 | 0.34 | −0.03 | −0.14 | 0.16 | 0.26 | −0.12 | 0.03 | −0.15 | −0.41 | −0.19 | 0.20 | −0.26 | −0.04 | 0.29 |
| 1793 | −0.29 | −0.02 | 1.00 | −0.26 | 0.18 | −0.16 | −0.21 | −0.03 | −0.10 | 0.72 | −0.34 | −0.48 | −0.47 | −0.27 | 0.44 | −0.31 | 0.03 | −0.44 | −0.39 | 0.01 |
| 1809 | 0.47 | −0.25 | −0.26 | 1.00 | −0.41 | −0.06 | 0.06 | 0.25 | 0.74 | −0.31 | 0.10 | 0.73 | 0.31 | 0.08 | 0.49 | −0.13 | −0.25 | 0.83 | 0.18 | −0.55 |
| 1825 | 0.02 | 0.41 | 0.18 | −0.41 | 1.00 | 0.38 | 0.30 | −0.06 | −0.24 | 0.58 | −0.33 | −0.53 | −0.50 | −0.33 | −0.46 | −0.28 | −0.20 | −0.52 | −0.31 | 0.54 |
| 1850 | −0.44 | 0.56 | −0.16 | −0.06 | 0.38 | 1.00 | 0.73 | −0.39 | −0.29 | 0.20 | 0.63 | −0.27 | −0.01 | −0.53 | −0.45 | −0.39 | 0.26 | −0.30 | 0.23 | 0.32 |
| 1866 | −0.31 | 0.34 | −0.21 | 0.06 | 0.30 | 0.73 | 1.00 | 0.01 | −0.39 | −0.08 | 0.58 | −0.18 | −0.01 | 0.00 | −0.57 | −0.15 | −0.25 | −0.23 | 0.37 | −0.19 |
| 1905 | 0.68 | −0.03 | −0.03 | 0.25 | −0.06 | −0.39 | 0.01 | 1.00 | 0.41 | −0.36 | −0.23 | 0.38 | 0.27 | 0.80 | −0.10 | 0.20 | −0.27 | 0.36 | 0.14 | −0.53 |
| 1955 | 0.76 | −0.14 | −0.10 | 0.74 | −0.24 | −0.29 | −0.39 | 0.41 | 1.00 | −0.22 | −0.28 | 0.68 | 0.26 | 0.10 | 0.50 | −0.14 | 0.00 | 0.78 | −0.12 | −0.21 |
| 1996 | −0.33 | 0.16 | 0.72 | −0.31 | 0.58 | 0.20 | −0.08 | −0.36 | −0.22 | 1.00 | −0.38 | −0.54 | −0.63 | −0.62 | 0.23 | −0.43 | 0.00 | −0.50 | −0.52 | 0.42 |
| 2012 | −0.51 | 0.26 | −0.34 | 0.10 | −0.33 | 0.63 | 0.58 | −0.23 | −0.28 | −0.38 | 1.00 | 0.15 | 0.59 | −0.08 | −0.28 | −0.25 | 0.40 | 0.05 | 0.76 | −0.16 |
| 2028 | 0.58 | −0.12 | −0.48 | 0.73 | −0.53 | −0.27 | −0.18 | 0.38 | 0.68 | −0.54 | 0.15 | 1.00 | 0.69 | 0.41 | 0.33 | −0.11 | −0.01 | 0.96 | 0.36 | −0.41 |
| 2041 | 0.19 | 0.03 | −0.47 | 0.31 | −0.50 | −0.01 | −0.01 | 0.27 | 0.26 | −0.63 | 0.59 | 0.69 | 1.00 | 0.39 | 0.00 | −0.23 | 0.36 | 0.59 | 0.81 | −0.24 |
| 2067 | 0.49 | −0.15 | −0.27 | 0.08 | −0.33 | −0.53 | 0.00 | 0.80 | 0.10 | −0.62 | −0.08 | 0.41 | 0.39 | 1.00 | −0.20 | 0.41 | −0.34 | 0.32 | 0.28 | −0.57 |
| 2101 | 0.13 | −0.41 | 0.44 | 0.49 | −0.46 | −0.45 | −0.57 | −0.10 | 0.50 | 0.23 | −0.28 | 0.33 | 0.00 | −0.20 | 1.00 | −0.22 | −0.01 | 0.42 | −0.22 | −0.23 |
| 2142 | 0.17 | −0.19 | −0.31 | −0.13 | −0.28 | −0.39 | −0.15 | 0.20 | −0.14 | −0.43 | −0.25 | −0.11 | −0.23 | 0.41 | −0.22 | 1.00 | −0.18 | −0.11 | −0.31 | −0.29 |
| 2158 | −0.32 | 0.20 | 0.03 | −0.25 | −0.20 | 0.26 | −0.25 | −0.27 | 0.00 | 0.00 | 0.40 | −0.01 | 0.36 | −0.34 | −0.01 | −0.18 | 1.00 | −0.09 | 0.15 | 0.37 |
| 2174 | 0.63 | −0.26 | −0.44 | 0.83 | −0.52 | −0.30 | −0.23 | 0.36 | 0.78 | −0.50 | 0.05 | 0.96 | 0.59 | 0.32 | 0.42 | −0.11 | −0.09 | 1.00 | 0.31 | −0.42 |
| 2229 | −0.17 | −0.04 | −0.39 | 0.18 | −0.31 | 0.23 | 0.37 | 0.14 | −0.12 | −0.52 | 0.76 | 0.36 | 0.81 | 0.28 | −0.22 | −0.31 | 0.15 | 0.31 | 1.00 | −0.24 |
| 2304 | −0.21 | 0.29 | 0.01 | −0.55 | 0.54 | 0.32 | −0.19 | −0.53 | −0.21 | 0.42 | −0.16 | −0.41 | −0.24 | −0.57 | −0.23 | −0.29 | 0.37 | −0.42 | −0.24 | 1.00 |

TABLE 31
Correlation matrix for acidic glycans derived from embryonic stem cells.

| | 1354 | 1362 | 1403 | 1475 | 1500 | 1516 | 1541 | 1549 | 1557 | 1565 | 1637 | 1678 | 1703 | 1711 | 1719 | 1727 | 1744 | 1768 |
|---|---|---|---|---|---|---|---|---|---|---|---|---|---|---|---|---|---|---|
| 1354 | 1.00 | 0.00 | −0.15 | 0.11 | 0.63 | 0.75 | 0.91 | −0.18 | 0.37 | 0.25 | 0.13 | 0.27 | 0.46 | −0.54 | 0.61 | 0.05 | 0.46 | 0.33 |
| 1362 | 0.00 | 1.00 | 0.47 | −0.16 | −0.21 | 0.13 | 0.09 | 0.03 | 0.06 | 0.22 | −0.20 | −0.11 | 0.04 | 0.12 | 0.11 | 0.02 | 0.07 | 0.37 |
| 1403 | −0.15 | 0.47 | 1.00 | −0.14 | −0.14 | 0.09 | 0.05 | 0.19 | 0.30 | 0.11 | −0.16 | −0.20 | 0.21 | −0.16 | 0.17 | 0.05 | 0.06 | 0.28 |
| 1475 | 0.11 | −0.16 | −0.14 | 1.00 | 0.56 | 0.11 | 0.12 | −0.23 | −0.17 | −0.18 | 0.95 | 0.70 | 0.34 | 0.32 | −0.01 | 0.29 | 0.41 | −0.21 |
| 1500 | 0.63 | −0.21 | −0.14 | 0.56 | 1.00 | 0.52 | 0.65 | −0.15 | −0.04 | 0.16 | 0.65 | 0.58 | 0.69 | −0.14 | 0.57 | 0.09 | 0.47 | −0.04 |
| 1516 | 0.75 | 0.13 | 0.09 | 0.11 | 0.52 | 1.00 | 0.63 | −0.13 | −0.04 | 0.33 | 0.11 | 0.28 | 0.36 | −0.30 | 0.48 | 0.07 | 0.71 | 0.56 |
| 1541 | 0.91 | 0.09 | 0.05 | 0.12 | 0.65 | 0.63 | 1.00 | −0.13 | 0.39 | 0.17 | 0.14 | 0.14 | 0.52 | −0.56 | 0.56 | −0.05 | 0.45 | 0.25 |
| 1549 | −0.18 | 0.03 | 0.19 | −0.23 | −0.15 | −0.13 | −0.13 | 1.00 | −0.03 | 0.26 | −0.26 | −0.20 | −0.20 | −0.03 | −0.20 | 0.17 | −0.15 | −0.09 |
| 1557 | 0.37 | 0.06 | 0.30 | −0.17 | −0.04 | −0.04 | 0.39 | −0.03 | 1.00 | 0.19 | −0.20 | 0.05 | 0.31 | −0.46 | 0.48 | 0.22 | −0.20 | 0.22 |
| 1565 | 0.25 | 0.22 | 0.11 | −0.18 | 0.16 | 0.33 | 0.17 | 0.26 | 0.19 | 1.00 | −0.14 | 0.33 | 0.27 | −0.17 | 0.25 | 0.56 | 0.00 | 0.32 |
| 1637 | 0.13 | −0.20 | −0.16 | 0.95 | 0.65 | 0.11 | 0.14 | −0.26 | −0.20 | −0.14 | 1.00 | 0.73 | 0.32 | 0.34 | 0.05 | 0.28 | 0.46 | −0.25 |
| 1678 | 0.27 | −0.11 | −0.20 | 0.70 | 0.58 | 0.28 | 0.14 | −0.20 | 0.05 | 0.33 | 0.73 | 1.00 | 0.47 | 0.11 | 0.29 | 0.69 | 0.41 | 0.25 |
| 1703 | 0.46 | 0.04 | 0.21 | 0.34 | 0.69 | 0.36 | 0.52 | −0.20 | 0.31 | 0.27 | 0.32 | 0.47 | 1.00 | −0.14 | 0.80 | 0.06 | 0.15 | 0.26 |
| 1711 | −0.54 | 0.12 | −0.16 | 0.32 | −0.14 | −0.30 | −0.56 | −0.03 | −0.46 | −0.17 | 0.34 | 0.11 | −0.14 | 1.00 | −0.28 | −0.08 | −0.07 | −0.27 |
| 1719 | 0.61 | 0.11 | 0.17 | −0.01 | 0.57 | 0.48 | 0.56 | −0.20 | 0.48 | 0.25 | 0.05 | 0.29 | 0.80 | −0.28 | 1.00 | −0.10 | 0.17 | 0.35 |
| 1727 | 0.05 | 0.02 | 0.05 | 0.29 | 0.09 | 0.07 | −0.05 | 0.17 | 0.22 | 0.56 | 0.28 | 0.69 | 0.06 | −0.08 | −0.10 | 1.00 | 0.03 | 0.24 |
| 1744 | 0.46 | 0.07 | 0.06 | 0.41 | 0.47 | 0.71 | 0.45 | −0.15 | −0.20 | 0.00 | 0.46 | 0.41 | 0.15 | −0.07 | 0.17 | 0.03 | 1.00 | 0.48 |
| 1768 | 0.33 | 0.37 | 0.28 | −0.21 | −0.04 | 0.56 | 0.25 | −0.09 | 0.22 | 0.32 | −0.25 | 0.25 | 0.26 | −0.27 | 0.35 | 0.24 | 0.48 | 1.00 |
| 1791 | 0.91 | −0.11 | −0.02 | 0.21 | 0.74 | 0.77 | 0.92 | −0.12 | 0.21 | 0.17 | 0.24 | 0.18 | 0.47 | −0.49 | 0.53 | −0.06 | 0.51 | 0.13 |
| 1799 | 0.12 | 0.00 | −0.06 | 0.78 | 0.35 | −0.08 | 0.21 | −0.16 | 0.09 | −0.03 | 0.74 | 0.58 | 0.27 | 0.16 | −0.13 | 0.36 | 0.36 | −0.02 |
| 1840 | −0.13 | −0.06 | −0.12 | 0.46 | 0.17 | −0.04 | −0.20 | −0.25 | −0.13 | 0.29 | 0.52 | 0.67 | 0.09 | 0.25 | −0.14 | 0.48 | 0.33 | 0.20 |
| 1865 | 0.65 | 0.23 | 0.51 | 0.16 | 0.55 | 0.46 | 0.75 | −0.08 | 0.68 | 0.33 | 0.18 | 0.29 | 0.71 | −0.47 | 0.74 | 0.17 | 0.28 | 0.30 |
| 1873 | 0.17 | 0.01 | 0.02 | 0.47 | 0.35 | 0.24 | 0.11 | −0.14 | 0.22 | 0.21 | 0.46 | 0.81 | 0.35 | −0.03 | 0.29 | 0.58 | 0.44 | 0.48 |
| 1889 | 0.23 | 0.04 | 0.13 | 0.22 | 0.25 | 0.25 | 0.11 | 0.03 | 0.43 | 0.52 | 0.18 | 0.72 | 0.45 | −0.27 | 0.44 | 0.69 | 0.17 | 0.50 |
| 1906 | 0.42 | 0.25 | 0.07 | 0.58 | 0.73 | 0.40 | 0.44 | −0.20 | 0.07 | 0.26 | 0.68 | 0.77 | 0.59 | 0.08 | 0.48 | 0.45 | 0.50 | 0.25 |
| 1914 | 0.09 | 0.27 | 0.23 | 0.40 | 0.48 | 0.30 | 0.13 | 0.07 | 0.02 | 0.36 | 0.46 | 0.64 | 0.39 | 0.21 | 0.37 | 0.47 | 0.39 | 0.25 |
| 1930 | −0.25 | 0.14 | 0.26 | −0.74 | −0.74 | −0.17 | −0.24 | 0.36 | 0.24 | 0.30 | −0.81 | −0.56 | −0.51 | −0.36 | −0.31 | 0.01 | −0.32 | 0.18 |
| 1946 | −0.38 | 0.07 | 0.41 | −0.42 | −0.45 | −0.09 | −0.26 | 0.44 | −0.06 | 0.32 | −0.48 | −0.35 | −0.33 | −0.32 | −0.31 | 0.09 | −0.09 | 0.08 |
| 1947 | 0.47 | −0.01 | −0.29 | 0.58 | 0.73 | 0.27 | 0.35 | −0.36 | 0.04 | 0.02 | 0.71 | 0.74 | 0.46 | 0.10 | 0.47 | 0.29 | 0.34 | 0.02 |
| 2002 | 0.07 | −0.05 | −0.05 | 0.57 | 0.52 | 0.28 | −0.04 | −0.37 | −0.22 | 0.17 | 0.62 | 0.82 | 0.40 | 0.16 | 0.22 | 0.47 | 0.41 | 0.28 |
| 2010 | 0.81 | 0.16 | 0.09 | 0.24 | 0.45 | 0.60 | 0.78 | −0.19 | 0.58 | 0.20 | 0.26 | 0.35 | 0.35 | −0.37 | 0.56 | 0.15 | 0.56 | 0.35 |
| 2011 | −0.15 | −0.24 | −0.17 | 0.08 | 0.33 | −0.16 | −0.12 | −0.21 | −0.30 | −0.17 | 0.20 | 0.14 | 0.30 | −0.03 | 0.29 | −0.34 | 0.06 | −0.15 |
| 2018 | 0.18 | −0.09 | −0.06 | −0.20 | −0.24 | 0.08 | −0.14 | −0.08 | 0.52 | 0.22 | −0.22 | 0.26 | 0.03 | −0.20 | 0.28 | 0.37 | −0.16 | 0.40 |
| 2035 | 0.19 | 0.00 | −0.04 | 0.58 | 0.49 | 0.45 | 0.10 | −0.24 | −0.18 | 0.46 | 0.65 | 0.74 | 0.35 | 0.37 | 0.16 | 0.47 | 0.54 | 0.23 |
| 2052 | 0.10 | −0.14 | −0.24 | 0.50 | 0.66 | 0.05 | 0.05 | 0.00 | −0.11 | 0.39 | 0.56 | 0.76 | 0.56 | 0.22 | 0.31 | 0.50 | 0.02 | −0.06 |
| 2068 | 0.62 | 0.01 | −0.06 | 0.61 | 0.76 | 0.56 | 0.60 | −0.25 | 0.05 | 0.29 | 0.70 | 0.69 | 0.51 | −0.03 | 0.35 | 0.30 | 0.70 | 0.24 |
| 2076 | −0.46 | 0.23 | 0.10 | −0.77 | −0.84 | −0.37 | −0.43 | 0.38 | 0.04 | 0.10 | −0.80 | −0.71 | −0.68 | 0.03 | −0.44 | −0.19 | −0.44 | −0.02 |
| 2092 | −0.53 | 0.02 | −0.06 | −0.58 | −0.51 | −0.36 | −0.52 | 0.27 | −0.30 | 0.15 | −0.57 | −0.43 | −0.49 | −0.12 | −0.35 | −0.10 | −0.42 | −0.16 |
| 2117 | −0.51 | −0.13 | −0.32 | 0.31 | −0.07 | −0.27 | −0.57 | 0.02 | −0.51 | −0.38 | 0.36 | 0.25 | −0.31 | 0.71 | −0.31 | 0.06 | 0.08 | −0.17 |
| 2133 | 0.32 | 0.19 | 0.15 | 0.64 | 0.30 | 0.27 | 0.34 | −0.26 | 0.26 | 0.20 | 0.56 | 0.66 | 0.32 | −0.09 | 0.04 | 0.68 | 0.33 | 0.25 |
| 2156 | 0.81 | 0.10 | 0.16 | 0.24 | 0.66 | 0.53 | 0.79 | −0.12 | 0.64 | 0.41 | 0.29 | 0.48 | 0.63 | −0.46 | 0.75 | 0.31 | 0.32 | 0.25 |
| 2157 | 0.01 | −0.28 | −0.20 | 0.00 | −0.07 | −0.07 | −0.17 | −0.24 | 0.05 | −0.16 | 0.02 | 0.12 | 0.11 | 0.00 | 0.09 | −0.06 | −0.06 | 0.16 |
| 2164 | 0.07 | 0.18 | 0.04 | −0.30 | 0.10 | 0.24 | 0.13 | −0.16 | −0.22 | 0.34 | −0.17 | 0.06 | 0.15 | −0.19 | 0.16 | 0.00 | 0.28 | 0.43 |
| 2221 | −0.23 | 0.07 | 0.22 | −0.29 | −0.48 | −0.15 | −0.23 | −0.16 | 0.06 | −0.18 | −0.31 | −0.44 | −0.33 | 0.13 | −0.26 | −0.21 | −0.20 | −0.03 |
| 2222 | −0.37 | −0.23 | −0.22 | −0.39 | −0.37 | −0.37 | −0.37 | 0.40 | −0.33 | −0.13 | −0.41 | −0.52 | −0.47 | −0.06 | −0.42 | −0.31 | −0.37 | −0.37 |
| 2230 | 0.60 | 0.10 | 0.17 | 0.24 | 0.68 | 0.41 | 0.66 | −0.22 | 0.43 | 0.10 | 0.35 | 0.38 | 0.71 | −0.21 | 0.85 | −0.12 | 0.46 | 0.24 |
| 2237 | −0.33 | 0.27 | 0.54 | −0.40 | −0.48 | −0.15 | −0.27 | 0.23 | 0.10 | 0.15 | −0.45 | −0.29 | −0.35 | −0.44 | −0.26 | 0.26 | −0.26 | 0.10 |
| 2238 | −0.14 | −0.21 | −0.12 | 0.15 | 0.26 | −0.16 | −0.10 | 0.05 | −0.17 | −0.20 | 0.20 | −0.02 | 0.22 | 0.24 | 0.18 | −0.38 | −0.05 | −0.31 |
| 2239 | −0.10 | 0.32 | −0.17 | 0.07 | 0.26 | −0.26 | −0.04 | −0.21 | −0.05 | 0.03 | 0.16 | 0.21 | 0.36 | 0.34 | 0.35 | −0.03 | −0.31 | −0.18 |
| 2246 | 0.59 | −0.09 | −0.06 | 0.25 | 0.54 | 0.70 | 0.46 | −0.08 | −0.11 | 0.20 | 0.28 | 0.13 | 0.18 | −0.09 | 0.32 | −0.04 | 0.35 | −0.16 |
| 2253 | −0.24 | 0.33 | 0.64 | −0.36 | −0.38 | −0.09 | −0.13 | 0.03 | 0.14 | 0.20 | −0.41 | −0.34 | −0.15 | −0.46 | −0.11 | 0.02 | −0.16 | 0.10 |
| 2254 | −0.19 | 0.08 | 0.29 | −0.24 | −0.34 | −0.11 | −0.12 | −0.01 | 0.00 | 0.04 | −0.27 | −0.28 | −0.17 | −0.37 | −0.17 | −0.04 | −0.14 | −0.05 |
| 2263 | −0.43 | −0.29 | −0.32 | 0.17 | 0.03 | −0.34 | −0.43 | 0.53 | −0.56 | −0.11 | 0.17 | 0.02 | −0.22 | 0.32 | −0.38 | 0.04 | −0.21 | −0.48 |
| 2279 | 0.04 | 0.17 | −0.04 | 0.42 | 0.09 | 0.02 | 0.05 | −0.33 | −0.01 | −0.30 | 0.41 | 0.23 | 0.05 | 0.50 | −0.07 | 0.07 | 0.19 | 0.03 |
| 2280 | 0.03 | 0.19 | 0.04 | 0.46 | −0.01 | 0.22 | −0.05 | −0.14 | −0.17 | 0.02 | 0.37 | 0.32 | 0.00 | 0.42 | −0.26 | 0.35 | 0.25 | 0.17 |
| 2295 | −0.09 | 0.23 | 0.19 | −0.50 | −0.13 | −0.07 | 0.04 | 0.32 | 0.07 | 0.37 | −0.42 | −0.10 | −0.19 | −0.42 | −0.08 | 0.33 | −0.12 | 0.23 |
| 2321 | −0.02 | −0.19 | −0.13 | 0.08 | 0.19 | 0.05 | 0.00 | −0.16 | −0.23 | −0.33 | 0.01 | 0.06 | 0.36 | −0.17 | 0.30 | −0.34 | 0.07 | 0.10 |
| 2367 | −0.45 | −0.17 | −0.10 | −0.59 | −0.75 | −0.42 | −0.47 | 0.10 | −0.16 | −0.37 | −0.63 | −0.82 | −0.70 | 0.09 | −0.57 | −0.49 | −0.46 | −0.27 |
| 2368 | −0.18 | −0.19 | −0.17 | −0.33 | −0.47 | −0.16 | −0.29 | 0.11 | −0.05 | −0.20 | −0.33 | −0.30 | −0.60 | −0.20 | −0.38 | 0.02 | −0.19 | −0.09 |
| 2383 | −0.27 | 0.08 | 0.03 | −0.34 | −0.43 | −0.07 | −0.23 | 0.45 | −0.17 | 0.27 | −0.39 | −0.23 | −0.37 | −0.13 | −0.32 | 0.10 | −0.06 | 0.07 |
| 2384 | −0.20 | −0.10 | −0.08 | −0.18 | −0.09 | −0.10 | −0.17 | −0.17 | −0.24 | −0.02 | −0.25 | −0.16 | −0.02 | −0.30 | −0.03 | −0.28 | −0.09 | −0.02 |
| 2390 | −0.12 | −0.01 | −0.02 | −0.44 | −0.32 | −0.11 | −0.10 | 0.72 | −0.21 | 0.16 | −0.48 | −0.42 | −0.39 | −0.36 | −0.37 | −0.04 | −0.14 | −0.02 |
| 2400 | −0.21 | −0.13 | −0.09 | 0.19 | 0.11 | −0.22 | −0.20 | −0.11 | −0.16 | −0.52 | 0.16 | 0.09 | 0.04 | 0.09 | 0.04 | 0.00 | −0.23 | −0.22 |
| 2408 | −0.34 | −0.08 | −0.10 | −0.28 | −0.36 | −0.25 | −0.32 | 0.43 | −0.26 | −0.24 | −0.36 | −0.48 | −0.24 | 0.15 | −0.21 | −0.43 | −0.26 | −0.19 |
| 2425 | −0.18 | −0.30 | −0.38 | 0.58 | 0.28 | −0.15 | −0.24 | −0.19 | −0.35 | −0.40 | 0.61 | 0.42 | 0.02 | 0.37 | −0.06 | 0.01 | 0.16 | −0.30 |
| 2441 | −0.53 | −0.18 | −0.23 | −0.33 | −0.52 | −0.59 | −0.43 | 0.27 | −0.34 | −0.48 | −0.36 | −0.75 | −0.58 | 0.34 | −0.62 | −0.53 | −0.51 | −0.57 |
| 2447 | 0.70 | 0.09 | 0.01 | −0.14 | 0.52 | 0.52 | 0.67 | −0.21 | 0.49 | 0.53 | 0.01 | 0.31 | 0.53 | −0.31 | 0.74 | 0.14 | 0.26 | 0.38 |
| 2448 | 0.46 | 0.17 | 0.20 | −0.21 | −0.04 | 0.18 | 0.45 | −0.11 | 0.90 | 0.29 | −0.25 | 0.17 | 0.32 | −0.43 | 0.51 | 0.26 | 0.03 | 0.56 |
| 2482 | 0.19 | −0.31 | −0.22 | 0.91 | 0.61 | 0.23 | 0.21 | −0.27 | −0.16 | −0.25 | 0.88 | 0.70 | 0.34 | 0.21 | 0.08 | 0.21 | 0.57 | −0.05 |
| 2512 | −0.09 | 0.86 | 0.86 | −0.17 | −0.21 | 0.13 | 0.08 | 0.13 | 0.21 | 0.20 | −0.21 | −0.18 | 0.15 | −0.03 | 0.16 | 0.04 | 0.08 | 0.38 |
| 2513 | 0.09 | 0.85 | −0.06 | −0.10 | −0.15 | 0.09 | 0.07 | −0.08 | −0.11 | 0.18 | −0.13 | −0.01 | −0.07 | 0.23 | 0.02 | 0.00 | 0.05 | 0.25 |
| 2521 | 0.34 | −0.17 | −0.22 | 0.20 | 0.57 | 0.28 | 0.33 | −0.36 | −0.06 | 0.50 | 0.21 | 0.39 | 0.60 | −0.24 | 0.41 | 0.10 | 0.05 | −0.01 |
| 2522 | 0.34 | −0.01 | 0.03 | −0.05 | 0.12 | −0.16 | 0.35 | −0.14 | 0.89 | 0.16 | −0.06 | 0.19 | 0.49 | −0.28 | 0.60 | 0.13 | −0.33 | 0.07 |
| 2528 | −0.15 | 0.40 | 0.27 | −0.27 | −0.38 | −0.07 | −0.09 | −0.03 | −0.04 | 0.11 | −0.30 | −0.28 | −0.19 | −0.27 | −0.15 | −0.03 | −0.11 | 0.05 |
| 2529 | −0.20 | 0.29 | 0.47 | −0.15 | −0.24 | 0.26 | −0.09 | 0.02 | 0.05 | 0.25 | −0.20 | 0.01 | 0.03 | 0.13 | 0.05 | 0.11 | 0.30 | 0.53 |
| 2544 | −0.19 | 0.09 | 0.31 | −0.24 | −0.34 | −0.11 | −0.12 | −0.01 | 0.01 | 0.04 | −0.27 | −0.29 | −0.17 | −0.37 | −0.17 | −0.04 | −0.14 | −0.05 |
| 2570 | 0.01 | −0.05 | −0.12 | −0.06 | −0.30 | −0.05 | −0.06 | −0.25 | 0.11 | −0.44 | −0.06 | −0.09 | −0.34 | 0.07 | −0.20 | −0.06 | 0.14 | 0.21 |
| 2571 | 0.15 | −0.15 | −0.11 | 0.15 | 0.12 | 0.14 | 0.06 | −0.22 | −0.01 | −0.48 | 0.12 | −0.13 | 0.02 | 0.24 | 0.19 | −0.36 | −0.01 | −0.24 |
| 2586 | −0.21 | 0.15 | 0.44 | −0.25 | 0.14 | −0.09 | −0.10 | 0.02 | 0.05 | 0.35 | −0.03 | 0.00 | 0.25 | 0.14 | 0.29 | 0.04 | −0.12 | 0.00 |
| 2587 | −0.24 | −0.27 | −0.24 | −0.23 | −0.29 | −0.28 | −0.25 | 0.29 | −0.25 | −0.33 | −0.23 | −0.43 | −0.49 | −0.10 | −0.37 | −0.35 | −0.16 | −0.34 |
| 2603 | −0.17 | −0.01 | 0.09 | 0.16 | 0.18 | −0.14 | −0.13 | −0.05 | −0.06 | −0.35 | 0.09 | 0.02 | 0.48 | 0.14 | 0.45 | −0.42 | −0.15 | −0.11 |
| 2644 | 0.11 | −0.13 | −0.10 | 0.36 | 0.17 | −0.05 | 0.13 | −0.31 | 0.10 | −0.36 | 0.36 | 0.10 | 0.13 | 0.36 | 0.02 | −0.12 | 0.09 | −0.14 |
| 2645 | −0.09 | −0.08 | −0.08 | −0.02 | −0.15 | −0.15 | −0.11 | −0.17 | −0.07 | −0.27 | −0.05 | −0.22 | −0.05 | 0.35 | −0.18 | −0.19 | −0.23 | −0.13 |
| 2660 | −0.15 | −0.09 | −0.06 | −0.20 | −0.12 | −0.15 | −0.14 | 0.97 | −0.11 | 0.23 | −0.22 | −0.15 | −0.26 | 0.01 | −0.24 | 0.16 | −0.16 | −0.16 |
| 2683 | 0.27 | −0.06 | −0.22 | 0.42 | 0.64 | 0.12 | 0.35 | −0.27 | 0.07 | 0.28 | 0.42 | 0.50 | 0.80 | 0.20 | 0.52 | 0.07 | 0.03 | −0.03 |
| 2714 | −0.15 | −0.09 | −0.06 | 0.58 | 0.15 | −0.15 | −0.14 | −0.08 | −0.11 | 0.24 | 0.46 | 0.46 | 0.31 | 0.32 | −0.24 | 0.48 | −0.16 | −0.16 |
| 2732 | −0.12 | −0.07 | −0.02 | −0.49 | −0.50 | −0.11 | −0.09 | 0.14 | −0.11 | −0.30 | −0.54 | −0.79 | −0.46 | 0.07 | −0.38 | −0.57 | −0.21 | −0.14 |
| 2733 | −0.14 | −0.31 | −0.19 | −0.08 | −0.09 | −0.27 | −0.26 | −0.05 | 0.16 | 0.21 | −0.04 | 0.11 | −0.06 | 0.10 | −0.13 | 0.36 | −0.49 | −0.20 |
| 2807 | −0.28 | −0.30 | −0.16 | −0.05 | −0.33 | −0.39 | −0.25 | 0.14 | −0.15 | −0.62 | −0.12 | −0.60 | −0.44 | 0.30 | −0.44 | −0.47 | −0.41 | −0.64 |
| 2878 | −0.21 | 0.07 | 0.34 | −0.02 | −0.16 | −0.14 | −0.20 | −0.04 | 0.12 | 0.31 | −0.11 | 0.03 | 0.02 | −0.22 | −0.16 | 0.29 | −0.23 | 0.02 |
| 2879 | −0.25 | 0.42 | 0.58 | −0.19 | −0.32 | −0.10 | −0.12 | 0.02 | 0.04 | −0.08 | −0.21 | −0.23 | −0.18 | −0.26 | −0.16 | 0.16 | −0.14 | 0.06 |
| 2880 | −0.15 | −0.09 | −0.06 | −0.20 | −0.30 | −0.15 | −0.14 | −0.08 | −0.11 | −0.36 | −0.22 | −0.47 | −0.26 | 0.12 | −0.24 | −0.43 | −0.16 | −0.16 |
| 2886 | 0.57 | −0.22 | −0.16 | 0.44 | 0.80 | 0.42 | 0.56 | −0.19 | 0.03 | 0.43 | 0.51 | 0.49 | 0.63 | −0.06 | 0.37 | 0.26 | 0.21 | −0.09 |
| 2936 | −0.10 | −0.07 | 0.00 | −0.27 | −0.37 | −0.12 | −0.17 | −0.08 | 0.07 | −0.28 | −0.29 | −0.40 | −0.23 | 0.22 | −0.14 | −0.31 | −0.20 | −0.01 |
| 2953 | −0.27 | −0.16 | −0.11 | −0.37 | −0.45 | −0.28 | −0.26 | 0.42 | −0.20 | −0.09 | −0.40 | −0.52 | −0.47 | −0.15 | −0.44 | −0.21 | −0.30 | −0.29 |
| 3024 | 0.54 | −0.15 | −0.11 | 0.01 | 0.17 | 0.55 | 0.22 | −0.13 | 0.24 | 0.30 | 0.01 | 0.23 | 0.09 | −0.29 | 0.38 | 0.21 | 0.12 | 0.12 |
| 3025 | 0.01 | −0.13 | −0.09 | −0.29 | −0.39 | −0.06 | −0.20 | −0.11 | 0.27 | 0.15 | −0.32 | 0.01 | −0.18 | −0.39 | 0.01 | 0.22 | −0.24 | 0.16 |
| 3098 | −0.17 | −0.10 | −0.05 | −0.25 | −0.34 | −0.18 | −0.17 | −0.09 | −0.10 | −0.33 | −0.28 | −0.50 | −0.30 | 0.23 | −0.27 | −0.42 | −0.20 | −0.16 |
| 3099 | −0.17 | −0.16 | −0.08 | −0.38 | −0.44 | −0.32 | −0.10 | 0.12 | 0.01 | −0.19 | −0.42 | −0.61 | −0.40 | −0.27 | −0.36 | −0.35 | −0.35 | −0.32 |
| 3170 | −0.15 | −0.09 | −0.06 | 0.09 | −0.01 | −0.15 | −0.14 | −0.08 | −0.11 | −0.36 | 0.11 | 0.08 | −0.26 | −0.01 | −0.24 | 0.29 | −0.16 | −0.16 |
| 3171 | −0.15 | 0.47 | 1.00 | −0.14 | −0.14 | 0.09 | 0.05 | 0.19 | 0.30 | 0.11 | −0.16 | −0.20 | 0.21 | −0.16 | 0.17 | 0.05 | 0.06 | 0.28 |
| 3172 | −0.19 | −0.12 | −0.08 | −0.26 | −0.39 | −0.20 | −0.19 | −0.10 | −0.15 | −0.35 | −0.29 | −0.53 | −0.34 | 0.00 | −0.32 | −0.43 | −0.22 | −0.21 |
| 3390 | −0.04 | −0.12 | −0.09 | −0.28 | −0.38 | −0.10 | −0.19 | −0.11 | 0.15 | 0.10 | −0.30 | −0.07 | −0.22 | −0.39 | −0.08 | 0.13 | −0.23 | 0.05 |
| 3463 | −0.21 | −0.13 | −0.09 | −0.28 | −0.27 | −0.22 | −0.20 | −0.11 | −0.16 | 0.10 | −0.31 | −0.28 | −0.36 | −0.42 | −0.34 | −0.08 | −0.23 | −0.22 |
| | 1791 | 1799 | 1840 | 1865 | 1873 | 1889 | 1906 | 1914 | 1930 | 1946 | 1947 | 2002 | 2010 | 2011 | 2018 | 2035 | 2052 | 2068 |
| 1354 | 0.91 | 0.12 | −0.13 | 0.65 | 0.17 | 0.23 | 0.42 | 0.09 | −0.25 | −0.38 | 0.47 | 0.07 | 0.81 | −0.15 | 0.18 | 0.19 | 0.10 | 0.62 |
| 1362 | −0.11 | 0.00 | −0.06 | 0.23 | 0.01 | 0.04 | 0.25 | 0.27 | 0.14 | 0.07 | −0.01 | −0.05 | 0.16 | −0.24 | −0.09 | 0.00 | −0.14 | 0.01 |
| 1403 | −0.02 | −0.06 | −0.12 | 0.51 | 0.02 | 0.13 | 0.07 | 0.23 | 0.26 | 0.41 | −0.29 | −0.05 | 0.09 | −0.17 | −0.06 | −0.04 | −0.24 | −0.06 |
| 1475 | 0.21 | 0.78 | 0.46 | 0.16 | 0.47 | 0.22 | 0.58 | 0.40 | −0.74 | −0.42 | 0.58 | 0.57 | 0.24 | 0.08 | −0.20 | 0.58 | 0.50 | 0.61 |
| 1500 | 0.74 | 0.35 | 0.17 | 0.55 | 0.35 | 0.25 | 0.73 | 0.48 | −0.74 | −0.45 | 0.73 | 0.52 | 0.45 | 0.33 | −0.24 | 0.49 | 0.66 | 0.76 |
| 1516 | 0.77 | −0.08 | −0.04 | 0.46 | 0.24 | 0.25 | 0.40 | 0.30 | −0.17 | −0.09 | 0.27 | 0.28 | 0.60 | −0.16 | 0.08 | 0.45 | 0.05 | 0.56 |
| 1541 | 0.92 | 0.21 | −0.20 | 0.75 | 0.11 | 0.11 | 0.44 | 0.13 | −0.24 | −0.26 | 0.35 | −0.04 | 0.78 | −0.12 | −0.14 | 0.10 | 0.05 | 0.60 |
| 1549 | −0.12 | −0.16 | −0.25 | −0.08 | −0.14 | 0.03 | −0.20 | 0.07 | 0.36 | 0.44 | −0.36 | −0.37 | −0.19 | −0.21 | −0.08 | −0.24 | 0.00 | −0.25 |
| 1557 | 0.21 | 0.09 | −0.13 | 0.68 | 0.22 | 0.43 | 0.07 | 0.02 | 0.24 | −0.06 | 0.04 | −0.22 | 0.58 | −0.30 | 0.52 | −0.18 | −0.11 | 0.05 |
| 1565 | 0.17 | −0.03 | 0.29 | 0.33 | 0.21 | 0.52 | 0.26 | 0.36 | 0.30 | 0.32 | 0.02 | 0.17 | 0.20 | −0.17 | 0.22 | 0.46 | 0.39 | 0.29 |
| 1637 | 0.24 | 0.74 | 0.52 | 0.18 | 0.46 | 0.18 | 0.68 | 0.46 | −0.81 | −0.48 | 0.71 | 0.62 | 0.26 | 0.20 | −0.22 | 0.65 | 0.56 | 0.70 |
| 1678 | 0.18 | 0.58 | 0.67 | 0.29 | 0.81 | 0.72 | 0.77 | 0.64 | −0.56 | −0.35 | 0.74 | 0.82 | 0.35 | 0.14 | 0.26 | 0.74 | 0.76 | 0.69 |
| 1703 | 0.47 | 0.27 | 0.09 | 0.71 | 0.35 | 0.45 | 0.59 | 0.39 | −0.51 | −0.33 | 0.46 | 0.40 | 0.35 | 0.30 | 0.03 | 0.35 | 0.56 | 0.51 |
| 1711 | −0.49 | 0.16 | 0.25 | −0.47 | −0.03 | −0.27 | 0.08 | 0.21 | −0.36 | −0.32 | 0.10 | 0.16 | −0.37 | −0.03 | −0.20 | 0.37 | 0.22 | −0.03 |
| 1719 | 0.53 | −0.13 | −0.14 | 0.74 | 0.29 | 0.44 | 0.48 | 0.37 | −0.31 | −0.31 | 0.47 | 0.22 | 0.56 | 0.29 | 0.28 | 0.16 | 0.31 | 0.35 |
| 1727 | −0.06 | 0.36 | 0.48 | 0.17 | 0.58 | 0.69 | 0.45 | 0.47 | 0.01 | 0.09 | 0.29 | 0.47 | 0.15 | −0.34 | 0.37 | 0.47 | 0.50 | 0.30 |
| 1744 | 0.51 | 0.36 | 0.33 | 0.28 | 0.44 | 0.17 | 0.50 | 0.39 | −0.32 | −0.09 | 0.34 | 0.41 | 0.56 | 0.06 | −0.16 | 0.54 | 0.02 | 0.70 |
| 1768 | 0.13 | −0.02 | 0.20 | 0.30 | 0.48 | 0.50 | 0.25 | 0.25 | 0.18 | 0.08 | 0.02 | 0.28 | 0.35 | −0.15 | 0.40 | 0.23 | −0.06 | 0.24 |
| 1791 | 1.00 | 0.10 | −0.21 | 0.66 | 0.05 | 0.07 | 0.40 | 0.13 | −0.31 | −0.25 | 0.37 | 0.04 | 0.72 | −0.10 | −0.12 | 0.23 | 0.09 | 0.62 |
| 1799 | 0.10 | 1.00 | 0.63 | 0.24 | 0.42 | 0.20 | 0.51 | 0.19 | −0.47 | −0.33 | 0.42 | 0.37 | 0.30 | −0.02 | −0.15 | 0.48 | 0.33 | 0.67 |
| 1840 | −0.21 | 0.63 | 1.00 | −0.05 | 0.60 | 0.44 | 0.36 | 0.32 | −0.21 | −0.12 | 0.35 | 0.72 | 0.03 | 0.20 | 0.16 | 0.68 | 0.49 | 0.53 |
| 1865 | 0.66 | 0.24 | −0.05 | 1.00 | 0.32 | 0.45 | 0.55 | 0.40 | −0.17 | −0.12 | 0.35 | 0.14 | 0.75 | −0.08 | 0.10 | 0.21 | 0.18 | 0.53 |
| 1873 | 0.05 | 0.42 | 0.60 | 0.32 | 1.00 | 0.85 | 0.57 | 0.74 | −0.28 | −0.14 | 0.48 | 0.77 | 0.39 | 0.05 | 0.30 | 0.47 | 0.57 | 0.43 |
| 1889 | 0.07 | 0.20 | 0.44 | 0.45 | 0.85 | 1.00 | 0.43 | 0.61 | 0.01 | 0.10 | 0.32 | 0.59 | 0.37 | 0.03 | 0.53 | 0.35 | 0.53 | 0.28 |
| 1906 | 0.40 | 0.51 | 0.36 | 0.55 | 0.57 | 0.43 | 1.00 | 0.72 | −0.71 | −0.45 | 0.84 | 0.63 | 0.49 | 0.14 | −0.07 | 0.65 | 0.65 | 0.79 |
| 1914 | 0.13 | 0.19 | 0.32 | 0.40 | 0.74 | 0.61 | 0.72 | 1.00 | −0.36 | −0.04 | 0.47 | 0.63 | 0.32 | 0.02 | −0.10 | 0.55 | 0.64 | 0.40 |
| 1930 | −0.31 | −0.47 | −0.21 | −0.17 | −0.28 | 0.01 | −0.71 | −0.36 | 1.00 | 0.77 | −0.82 | −0.55 | −0.15 | −0.34 | 0.23 | −0.45 | −0.59 | −0.59 |
| 1946 | −0.25 | −0.33 | −0.12 | −0.12 | −0.14 | 0.10 | −0.45 | −0.04 | 0.77 | 1.00 | −0.74 | −0.30 | −0.26 | −0.04 | −0.16 | −0.24 | −0.35 | −0.44 |
| 1947 | 0.37 | 0.42 | 0.35 | 0.35 | 0.48 | 0.32 | 0.84 | 0.47 | −0.82 | −0.74 | 1.00 | 0.64 | 0.42 | 0.28 | 0.14 | 0.49 | 0.66 | 0.69 |
| 2002 | 0.04 | 0.37 | 0.72 | 0.14 | 0.77 | 0.59 | 0.63 | 0.63 | −0.55 | −0.30 | 0.64 | 1.00 | 0.07 | 0.32 | 0.15 | 0.66 | 0.73 | 0.53 |
| 2010 | 0.72 | 0.30 | 0.03 | 0.75 | 0.39 | 0.37 | 0.49 | 0.32 | −0.15 | −0.26 | 0.42 | 0.07 | 1.00 | −0.27 | 0.22 | 0.30 | −0.02 | 0.63 |
| 2011 | −0.10 | −0.02 | 0.20 | −0.08 | 0.05 | 0.03 | 0.14 | 0.02 | −0.34 | −0.04 | 0.28 | 0.32 | −0.27 | 1.00 | −0.17 | −0.01 | 0.31 | 0.07 |
| 2018 | −0.12 | −0.15 | 0.16 | 0.10 | 0.30 | 0.53 | −0.07 | −0.10 | 0.23 | −0.16 | 0.14 | 0.15 | 0.22 | −0.17 | 1.00 | 0.03 | −0.01 | −0.06 |
| 2035 | 0.23 | 0.48 | 0.68 | 0.21 | 0.47 | 0.35 | 0.65 | 0.55 | −0.45 | −0.24 | 0.49 | 0.66 | 0.30 | −0.01 | 0.03 | 1.00 | 0.54 | 0.77 |
| 2052 | 0.09 | 0.33 | 0.49 | 0.18 | 0.57 | 0.53 | 0.65 | 0.64 | −0.59 | −0.35 | 0.66 | 0.73 | −0.02 | 0.31 | −0.01 | 0.54 | 1.00 | 0.45 |
| 2068 | 0.62 | 0.67 | 0.53 | 0.53 | 0.43 | 0.28 | 0.79 | 0.40 | −0.59 | −0.44 | 0.69 | 0.53 | 0.63 | 0.07 | −0.06 | 0.77 | 0.45 | 1.00 |
| 2076 | −0.52 | −0.54 | −0.30 | −0.44 | −0.42 | −0.29 | −0.74 | −0.34 | 0.86 | 0.57 | −0.79 | −0.64 | −0.34 | −0.35 | 0.05 | −0.51 | −0.58 | −0.73 |
| 2092 | −0.50 | −0.58 | −0.16 | −0.52 | −0.28 | −0.10 | −0.55 | −0.17 | 0.62 | 0.68 | −0.55 | −0.25 | −0.59 | 0.22 | −0.11 | −0.43 | −0.18 | −0.68 |
| 2117 | −0.48 | 0.01 | 0.18 | −0.57 | 0.28 | −0.06 | 0.13 | 0.38 | −0.38 | −0.22 | 0.22 | 0.32 | −0.36 | 0.12 | −0.13 | 0.17 | 0.27 | −0.15 |
| 2133 | 0.27 | 0.69 | 0.35 | 0.44 | 0.50 | 0.44 | 0.64 | 0.37 | −0.33 | −0.19 | 0.42 | 0.40 | 0.47 | −0.37 | 0.08 | 0.51 | 0.27 | 0.58 |
| 2156 | 0.75 | 0.26 | 0.04 | 0.90 | 0.41 | 0.53 | 0.63 | 0.41 | −0.27 | −0.29 | 0.58 | 0.23 | 0.86 | −0.08 | 0.26 | 0.32 | 0.32 | 0.65 |
| 2157 | −0.14 | 0.10 | 0.24 | −0.14 | −0.04 | −0.01 | −0.08 | −0.39 | −0.15 | −0.37 | 0.15 | 0.13 | −0.12 | 0.20 | 0.48 | 0.09 | −0.01 | 0.09 |
| 2164 | 0.05 | −0.11 | 0.14 | 0.03 | −0.07 | −0.01 | 0.28 | 0.04 | 0.04 | 0.23 | 0.05 | 0.11 | −0.06 | 0.42 | −0.17 | 0.22 | 0.02 | 0.23 |
| 2221 | −0.22 | −0.20 | −0.13 | −0.17 | −0.35 | −0.33 | −0.43 | −0.40 | 0.33 | 0.12 | −0.40 | −0.29 | −0.16 | −0.25 | 0.10 | −0.20 | −0.51 | −0.31 |
| 2222 | −0.31 | −0.38 | −0.26 | −0.55 | −0.48 | −0.39 | −0.63 | −0.44 | 0.40 | 0.34 | −0.50 | −0.43 | −0.54 | 0.10 | −0.17 | −0.48 | −0.25 | −0.54 |
| 2230 | 0.58 | 0.22 | 0.05 | 0.78 | 0.38 | 0.34 | 0.64 | 0.46 | −0.46 | −0.36 | 0.59 | 0.26 | 0.72 | 0.35 | 0.02 | 0.27 | 0.29 | 0.60 |
| 2237 | −0.30 | −0.37 | −0.17 | −0.01 | −0.03 | 0.19 | −0.30 | 0.01 | 0.64 | 0.74 | −0.47 | −0.11 | −0.25 | −0.13 | 0.08 | −0.40 | −0.30 | −0.51 |
| 2238 | −0.04 | 0.00 | 0.06 | −0.05 | 0.01 | −0.10 | −0.03 | 0.11 | −0.30 | −0.23 | 0.11 | 0.12 | −0.15 | 0.39 | −0.20 | 0.01 | 0.27 | 0.00 |
| 2239 | −0.18 | −0.02 | −0.02 | 0.05 | 0.09 | 0.04 | 0.46 | 0.37 | −0.49 | −0.43 | 0.52 | 0.23 | −0.15 | 0.38 | −0.17 | 0.02 | 0.55 | 0.01 |
| 2246 | 0.74 | −0.15 | −0.22 | 0.32 | −0.10 | −0.04 | 0.24 | 0.18 | −0.26 | −0.16 | 0.29 | 0.08 | 0.46 | −0.17 | −0.06 | 0.36 | 0.09 | 0.39 |
| 2253 | −0.18 | −0.22 | −0.02 | 0.16 | −0.13 | 0.12 | −0.32 | −0.12 | 0.63 | 0.73 | −0.50 | −0.11 | −0.13 | 0.05 | 0.00 | −0.28 | −0.36 | −0.32 |
| 2254 | −0.12 | −0.16 | −0.26 | −0.03 | −0.43 | −0.16 | −0.22 | −0.38 | 0.40 | 0.60 | −0.38 | −0.36 | −0.17 | 0.17 | −0.08 | −0.24 | −0.47 | −0.25 |
| 2263 | −0.31 | −0.06 | −0.07 | −0.53 | −0.13 | −0.17 | −0.10 | 0.06 | −0.16 | 0.10 | −0.04 | −0.01 | −0.57 | 0.21 | −0.32 | −0.08 | 0.31 | −0.25 |
| 2279 | 0.00 | 0.40 | 0.17 | 0.03 | 0.17 | −0.12 | 0.34 | 0.15 | −0.48 | −0.57 | 0.37 | 0.21 | 0.17 | −0.27 | −0.03 | 0.28 | 0.08 | 0.29 |
| 2280 | 0.00 | 0.43 | 0.30 | −0.05 | 0.15 | 0.02 | 0.25 | 0.09 | −0.25 | −0.27 | 0.15 | 0.26 | 0.08 | −0.46 | 0.08 | 0.48 | 0.06 | 0.33 |
| 2295 | −0.12 | −0.31 | −0.10 | 0.04 | 0.11 | 0.21 | 0.06 | 0.26 | 0.41 | 0.51 | −0.16 | −0.05 | −0.13 | 0.00 | −0.13 | −0.27 | 0.07 | −0.23 |
| 2321 | 0.01 | −0.15 | −0.23 | −0.08 | 0.05 | 0.08 | 0.02 | −0.05 | −0.26 | 0.06 | 0.05 | 0.12 | −0.21 | 0.67 | −0.13 | −0.23 | 0.07 | −0.15 |
| 2367 | −0.43 | −0.48 | −0.34 | −0.63 | −0.68 | −0.64 | −0.92 | −0.77 | 0.58 | 0.25 | −0.73 | −0.63 | −0.52 | −0.20 | 0.03 | −0.59 | −0.70 | −0.72 |
| 2368 | −0.22 | −0.36 | −0.29 | −0.42 | −0.28 | −0.22 | −0.41 | −0.37 | 0.38 | 0.23 | −0.29 | −0.35 | −0.19 | −0.20 | 0.21 | −0.35 | −0.45 | −0.43 |
| 2383 | −0.24 | −0.27 | −0.26 | −0.29 | −0.28 | −0.05 | −0.29 | −0.14 | 0.56 | 0.76 | −0.54 | −0.45 | −0.22 | −0.05 | −0.14 | −0.16 | −0.34 | −0.35 |
| 2384 | −0.16 | −0.26 | 0.06 | −0.21 | 0.07 | 0.15 | −0.38 | −0.09 | 0.30 | 0.48 | −0.33 | 0.13 | −0.31 | 0.51 | −0.15 | −0.30 | −0.02 | −0.35 |
| 2390 | −0.11 | −0.24 | −0.26 | −0.31 | −0.41 | −0.19 | −0.45 | −0.40 | 0.55 | 0.56 | −0.52 | −0.49 | −0.32 | −0.02 | −0.11 | −0.44 | −0.31 | −0.34 |
| 2400 | −0.17 | −0.21 | −0.32 | −0.16 | 0.18 | 0.04 | 0.13 | 0.23 | −0.41 | −0.23 | 0.27 | 0.23 | −0.30 | 0.20 | −0.09 | −0.33 | 0.26 | −0.35 |
| 2408 | −0.29 | −0.35 | −0.45 | −0.43 | −0.45 | −0.39 | −0.51 | −0.34 | 0.24 | 0.22 | −0.49 | −0.52 | −0.41 | 0.03 | −0.16 | −0.41 | −0.31 | −0.52 |
| 2425 | −0.13 | 0.25 | 0.22 | −0.26 | 0.30 | 0.03 | 0.28 | 0.28 | −0.59 | −0.37 | 0.46 | 0.41 | −0.11 | 0.33 | −0.13 | 0.23 | 0.37 | 0.13 |
| 2441 | −0.43 | −0.27 | −0.40 | −0.68 | −0.71 | −0.80 | −0.72 | −0.61 | 0.24 | 0.06 | −0.56 | −0.64 | −0.63 | −0.12 | −0.35 | −0.56 | −0.43 | −0.63 |
| 2447 | 0.60 | −0.02 | 0.06 | 0.68 | 0.23 | 0.32 | 0.54 | 0.33 | −0.16 | −0.29 | 0.47 | 0.15 | 0.66 | 0.02 | 0.22 | 0.36 | 0.28 | 0.56 |
| 2448 | 0.23 | 0.08 | −0.02 | 0.62 | 0.40 | 0.54 | 0.16 | 0.14 | 0.25 | −0.05 | 0.06 | −0.10 | 0.66 | −0.33 | 0.53 | −0.04 | −0.09 | 0.14 |
| 2482 | 0.30 | 0.65 | 0.38 | 0.16 | 0.59 | 0.27 | 0.56 | 0.47 | −0.71 | −0.36 | 0.54 | 0.57 | 0.32 | 0.15 | −0.22 | 0.53 | 0.45 | 0.58 |
| 2512 | −0.08 | −0.03 | −0.11 | 0.43 | 0.02 | 0.10 | 0.19 | 0.29 | 0.23 | 0.28 | −0.17 | −0.06 | 0.15 | −0.24 | −0.09 | −0.03 | −0.22 | −0.03 |
| 2513 | −0.12 | 0.04 | 0.00 | −0.05 | 0.00 | −0.03 | 0.24 | 0.16 | 0.00 | −0.16 | 0.16 | −0.02 | 0.12 | −0.17 | −0.06 | 0.02 | −0.01 | 0.05 |
| 2521 | 0.38 | 0.12 | 0.29 | 0.29 | 0.17 | 0.33 | 0.30 | 0.17 | −0.22 | 0.00 | 0.28 | 0.40 | 0.10 | 0.45 | −0.12 | 0.39 | 0.54 | 0.40 |
| 2522 | 0.15 | 0.13 | −0.08 | 0.59 | 0.24 | 0.44 | 0.17 | 0.07 | −0.02 | −0.28 | 0.25 | −0.11 | 0.47 | −0.04 | 0.46 | −0.14 | 0.16 | 0.08 |
| 2528 | −0.16 | −0.14 | −0.25 | −0.04 | −0.41 | −0.16 | −0.12 | −0.29 | 0.38 | 0.52 | −0.31 | −0.36 | −0.12 | 0.09 | −0.10 | −0.22 | −0.46 | −0.23 |
| 2529 | −0.13 | −0.16 | 0.04 | 0.13 | 0.30 | 0.25 | 0.02 | 0.45 | 0.32 | 0.49 | −0.39 | 0.06 | 0.11 | −0.26 | −0.10 | 0.26 | −0.16 | −0.08 |
| 2544 | −0.12 | −0.16 | −0.26 | −0.02 | −0.43 | −0.15 | −0.22 | −0.37 | 0.40 | 0.61 | −0.38 | −0.36 | −0.17 | 0.16 | −0.08 | −0.23 | −0.47 | −0.25 |
| 2570 | −0.12 | 0.05 | −0.02 | −0.19 | 0.01 | −0.17 | −0.11 | −0.28 | 0.00 | −0.25 | 0.02 | −0.10 | 0.11 | −0.21 | 0.27 | −0.15 | −0.41 | −0.06 |
| 2571 | 0.21 | −0.23 | −0.40 | 0.00 | −0.14 | −0.24 | −0.07 | −0.08 | −0.32 | −0.41 | 0.16 | −0.08 | 0.12 | −0.08 | 0.06 | −0.14 | −0.13 | −0.11 |
| 2586 | −0.12 | −0.16 | 0.16 | 0.26 | −0.07 | 0.00 | 0.29 | 0.30 | −0.07 | 0.06 | 0.14 | 0.17 | −0.14 | 0.33 | −0.09 | 0.25 | 0.26 | 0.13 |
| 2587 | −0.19 | −0.24 | −0.25 | −0.46 | −0.39 | −0.39 | −0.53 | −0.44 | 0.26 | 0.19 | −0.34 | −0.40 | −0.31 | 0.10 | −0.10 | −0.45 | −0.35 | −0.40 |
| 2603 | −0.12 | −0.16 | −0.24 | 0.07 | 0.09 | 0.13 | 0.01 | 0.15 | −0.32 | −0.10 | 0.09 | 0.13 | −0.20 | 0.57 | −0.07 | −0.24 | 0.21 | −0.25 |
| 2644 | 0.12 | 0.36 | 0.07 | 0.09 | 0.01 | −0.21 | 0.16 | −0.07 | −0.45 | −0.56 | 0.28 | 0.06 | 0.17 | −0.12 | −0.03 | 0.15 | 0.02 | 0.26 |
| 2645 | −0.10 | 0.02 | −0.09 | −0.20 | −0.31 | −0.39 | −0.19 | −0.36 | −0.14 | −0.35 | −0.07 | −0.14 | −0.22 | −0.21 | 0.01 | −0.07 | −0.13 | −0.09 |
| 2660 | −0.12 | −0.15 | −0.22 | −0.21 | −0.15 | 0.00 | −0.22 | 0.02 | 0.30 | 0.34 | −0.29 | −0.36 | −0.21 | −0.17 | −0.06 | −0.23 | 0.06 | −0.24 |
| 2683 | 0.29 | 0.36 | 0.20 | 0.37 | 0.28 | 0.27 | 0.55 | 0.40 | −0.58 | −0.39 | 0.46 | 0.35 | 0.17 | 0.28 | −0.22 | 0.46 | 0.69 | 0.47 |
| 2714 | −0.12 | 0.61 | 0.47 | −0.02 | 0.16 | 0.18 | 0.23 | 0.07 | −0.29 | −0.16 | 0.12 | 0.35 | −0.21 | −0.17 | −0.06 | 0.48 | 0.49 | 0.29 |
| 2732 | −0.07 | −0.34 | −0.44 | −0.36 | −0.72 | −0.70 | −0.73 | −0.72 | 0.43 | 0.12 | −0.64 | −0.70 | −0.24 | −0.32 | −0.11 | −0.45 | −0.71 | −0.42 |
| 2733 | −0.20 | −0.05 | 0.20 | −0.14 | 0.03 | 0.09 | −0.10 | −0.05 | 0.03 | −0.22 | 0.06 | 0.13 | −0.24 | −0.25 | 0.33 | 0.11 | 0.31 | −0.10 |
| 2807 | −0.14 | −0.19 | −0.53 | −0.43 | −0.65 | −0.73 | −0.59 | −0.57 | 0.03 | −0.17 | −0.36 | −0.60 | −0.33 | −0.31 | −0.19 | −0.46 | −0.45 | −0.48 |
| 2878 | −0.19 | 0.08 | 0.41 | 0.09 | 0.22 | 0.37 | −0.24 | 0.03 | 0.39 | 0.37 | −0.28 | 0.29 | −0.18 | −0.07 | 0.15 | 0.01 | 0.13 | −0.12 |
| 2879 | −0.18 | −0.20 | −0.34 | 0.09 | −0.18 | −0.06 | 0.03 | 0.02 | 0.24 | 0.44 | −0.20 | −0.16 | −0.16 | −0.11 | −0.13 | −0.29 | −0.33 | −0.31 |
| 2880 | −0.12 | −0.15 | −0.22 | −0.29 | −0.45 | −0.54 | −0.41 | −0.48 | 0.09 | −0.16 | −0.29 | −0.36 | −0.21 | −0.17 | −0.06 | −0.23 | −0.40 | −0.24 |
| 2886 | 0.66 | 0.40 | 0.25 | 0.47 | 0.10 | 0.13 | 0.58 | 0.22 | −0.54 | −0.40 | 0.54 | 0.35 | 0.33 | 0.04 | −0.16 | 0.62 | 0.59 | 0.76 |
| 2936 | −0.15 | −0.19 | −0.18 | −0.21 | −0.35 | −0.36 | −0.42 | −0.48 | 0.15 | −0.17 | −0.27 | −0.31 | −0.14 | −0.23 | 0.23 | −0.22 | −0.41 | −0.26 |
| 2953 | −0.22 | −0.27 | −0.41 | −0.44 | −0.67 | −0.47 | −0.55 | −0.61 | 0.44 | 0.42 | −0.53 | −0.66 | −0.39 | −0.03 | −0.11 | −0.42 | −0.49 | −0.44 |
| 3024 | 0.45 | −0.25 | −0.12 | 0.26 | 0.02 | 0.28 | 0.07 | −0.05 | 0.04 | −0.11 | 0.24 | 0.07 | 0.45 | −0.19 | 0.62 | 0.23 | −0.04 | 0.19 |
| 3025 | −0.17 | −0.22 | −0.06 | −0.10 | −0.14 | 0.21 | −0.25 | −0.43 | 0.41 | 0.26 | −0.13 | −0.18 | −0.01 | 0.06 | 0.64 | −0.16 | −0.31 | −0.23 |
| 3098 | −0.15 | −0.18 | −0.17 | −0.31 | −0.42 | −0.49 | −0.50 | −0.49 | 0.16 | −0.08 | −0.35 | −0.33 | −0.24 | −0.15 | −0.03 | −0.27 | −0.40 | −0.29 |
| 3099 | −0.12 | −0.21 | −0.40 | −0.28 | −0.66 | −0.52 | −0.59 | −0.69 | 0.44 | 0.31 | −0.53 | −0.69 | −0.25 | −0.06 | −0.14 | −0.50 | −0.58 | −0.42 |
| 3170 | −0.12 | −0.15 | −0.22 | −0.18 | 0.15 | −0.02 | 0.15 | 0.20 | −0.24 | −0.16 | 0.23 | 0.18 | −0.21 | −0.17 | −0.06 | −0.23 | 0.14 | −0.24 |
| 3171 | −0.02 | −0.06 | −0.12 | 0.51 | 0.02 | 0.13 | 0.07 | 0.23 | 0.26 | 0.41 | −0.29 | −0.05 | 0.09 | −0.17 | −0.06 | −0.04 | −0.24 | −0.06 |
| 3172 | −0.16 | −0.20 | −0.29 | −0.36 | −0.60 | −0.60 | −0.49 | −0.63 | 0.20 | 0.03 | −0.38 | −0.48 | −0.28 | −0.07 | −0.08 | −0.30 | −0.53 | −0.32 |
| 3390 | −0.16 | −0.21 | −0.12 | −0.15 | −0.27 | 0.07 | −0.27 | −0.48 | 0.40 | 0.36 | −0.20 | −0.26 | −0.09 | 0.13 | 0.43 | −0.19 | −0.37 | −0.25 |
| 3463 | −0.17 | −0.21 | 0.10 | −0.27 | −0.18 | −0.02 | −0.50 | −0.34 | 0.53 | 0.58 | −0.41 | −0.07 | −0.30 | 0.26 | −0.09 | −0.33 | −0.20 | −0.34 |
| | 2076 | 2092 | 2117 | 2133 | 2156 | 2157 | 2164 | 2221 | 2222 | 2230 | 2237 | 2238 | 2239 | 2246 | 2253 | 2254 | 2263 | 2279 |
| 1354 | −0.46 | −0.53 | −0.51 | 0.32 | 0.81 | 0.01 | 0.07 | −0.23 | −0.37 | 0.60 | −0.33 | −0.14 | −0.10 | 0.59 | −0.24 | −0.19 | −0.43 | 0.04 |
| 1362 | 0.23 | 0.02 | −0.13 | 0.19 | 0.10 | −0.28 | 0.18 | 0.07 | −0.23 | 0.10 | 0.27 | −0.21 | 0.32 | −0.09 | 0.33 | 0.08 | −0.29 | 0.17 |
| 1403 | 0.10 | −0.06 | −0.32 | 0.15 | 0.16 | −0.20 | 0.04 | 0.22 | −0.22 | 0.17 | 0.54 | −0.12 | −0.17 | −0.06 | 0.64 | 0.29 | −0.32 | −0.04 |
| 1475 | −0.77 | −0.58 | 0.31 | 0.64 | 0.24 | 0.00 | −0.30 | −0.29 | −0.39 | 0.24 | −0.40 | 0.15 | 0.07 | 0.25 | −0.36 | −0.24 | 0.17 | 0.42 |
| 1500 | −0.84 | −0.51 | −0.07 | 0.30 | 0.66 | −0.07 | 0.10 | −0.48 | −0.37 | 0.68 | −0.48 | 0.26 | 0.26 | 0.54 | −0.38 | −0.34 | 0.03 | 0.09 |
| 1516 | −0.37 | −0.36 | −0.27 | 0.27 | 0.53 | −0.07 | 0.24 | −0.15 | −0.37 | 0.41 | −0.15 | −0.16 | −0.26 | 0.70 | −0.09 | −0.11 | −0.34 | 0.02 |
| 1541 | −0.43 | −0.52 | −0.57 | 0.34 | 0.79 | −0.17 | 0.13 | −0.23 | −0.37 | 0.66 | −0.27 | −0.10 | −0.04 | 0.46 | −0.13 | −0.12 | −0.43 | 0.05 |
| 1549 | 0.38 | 0.27 | 0.02 | −0.26 | −0.12 | −0.24 | −0.16 | −0.16 | 0.40 | −0.22 | 0.23 | 0.05 | −0.21 | −0.08 | 0.03 | −0.01 | 0.53 | −0.33 |
| 1557 | 0.04 | −0.30 | −0.51 | 0.26 | 0.64 | 0.05 | −0.22 | 0.06 | −0.33 | 0.43 | 0.10 | −0.17 | −0.05 | −0.11 | 0.14 | 0.00 | −0.56 | −0.01 |
| 1565 | 0.10 | 0.15 | −0.38 | 0.20 | 0.41 | −0.16 | 0.34 | −0.18 | −0.13 | 0.10 | 0.15 | −0.20 | 0.03 | 0.20 | 0.20 | 0.04 | −0.11 | −0.30 |
| 1637 | −0.80 | −0.57 | 0.36 | 0.56 | 0.29 | 0.02 | −0.17 | −0.31 | −0.41 | 0.35 | −0.45 | 0.20 | 0.16 | 0.28 | −0.41 | −0.27 | 0.17 | 0.41 |
| 1678 | −0.71 | −0.43 | 0.25 | 0.66 | 0.48 | 0.12 | 0.06 | −0.44 | −0.52 | 0.38 | −0.29 | −0.02 | 0.21 | 0.13 | −0.34 | −0.28 | 0.02 | 0.23 |
| 1703 | −0.68 | −0.49 | −0.31 | 0.32 | 0.63 | 0.11 | 0.15 | −0.33 | −0.47 | 0.71 | −0.35 | 0.22 | 0.36 | 0.18 | −0.15 | −0.17 | −0.22 | 0.05 |
| 1711 | 0.03 | −0.12 | 0.71 | −0.09 | −0.46 | 0.00 | −0.19 | 0.13 | −0.06 | −0.21 | −0.44 | 0.24 | 0.34 | −0.09 | −0.46 | −0.37 | 0.32 | 0.50 |
| 1719 | −0.44 | −0.35 | −0.31 | 0.04 | 0.75 | 0.09 | 0.16 | −0.26 | −0.42 | 0.85 | −0.26 | 0.18 | 0.35 | 0.32 | −0.11 | −0.17 | −0.38 | −0.07 |
| 1727 | −0.19 | −0.10 | 0.06 | 0.68 | 0.31 | −0.06 | 0.00 | −0.21 | −0.31 | −0.12 | 0.26 | −0.38 | −0.03 | −0.04 | 0.02 | −0.04 | 0.04 | 0.07 |
| 1744 | −0.44 | −0.42 | 0.08 | 0.33 | 0.32 | −0.06 | 0.28 | −0.20 | −0.37 | 0.46 | −0.26 | −0.05 | −0.31 | 0.35 | −0.16 | −0.14 | −0.21 | 0.19 |
| 1768 | −0.02 | −0.16 | −0.17 | 0.25 | 0.25 | 0.16 | 0.43 | −0.03 | −0.37 | 0.24 | 0.10 | −0.31 | −0.18 | −0.16 | 0.10 | −0.05 | −0.48 | 0.03 |
| 1791 | −0.52 | −0.50 | −0.48 | 0.27 | 0.75 | −0.14 | 0.05 | −0.22 | −0.31 | 0.58 | −0.30 | −0.04 | −0.18 | 0.74 | −0.18 | −0.12 | −0.31 | 0.00 |
| 1799 | −0.54 | −0.58 | 0.01 | 0.69 | 0.26 | 0.10 | −0.11 | −0.20 | −0.38 | 0.22 | −0.37 | 0.00 | −0.02 | −0.15 | −0.22 | −0.16 | −0.06 | 0.40 |
| 1840 | −0.30 | −0.16 | 0.18 | 0.35 | 0.04 | 0.24 | 0.14 | −0.13 | −0.26 | 0.05 | −0.17 | 0.06 | −0.02 | −0.22 | −0.02 | −0.26 | −0.07 | 0.17 |
| 1865 | −0.44 | −0.52 | −0.57 | 0.44 | 0.90 | −0.14 | 0.03 | −0.17 | −0.55 | 0.78 | −0.01 | −0.05 | 0.05 | 0.32 | 0.16 | −0.03 | −0.53 | 0.03 |
| 1873 | −0.42 | −0.28 | 0.28 | 0.50 | 0.41 | −0.04 | −0.07 | −0.35 | −0.48 | 0.38 | −0.03 | 0.01 | 0.09 | −0.10 | −0.13 | −0.43 | −0.13 | 0.17 |
| 1889 | −0.29 | −0.10 | −0.06 | 0.44 | 0.53 | −0.01 | −0.01 | −0.33 | −0.39 | 0.34 | 0.19 | −0.10 | 0.04 | −0.04 | 0.12 | −0.16 | −0.17 | −0.12 |
| 1906 | −0.74 | −0.55 | 0.13 | 0.64 | 0.63 | −0.08 | 0.28 | −0.43 | −0.63 | 0.64 | −0.30 | −0.03 | 0.46 | 0.24 | −0.32 | −0.22 | −0.10 | 0.34 |
| 1914 | −0.34 | −0.17 | 0.38 | 0.37 | 0.41 | −0.39 | 0.04 | −0.40 | −0.44 | 0.46 | 0.01 | 0.11 | 0.37 | 0.18 | −0.12 | −0.38 | 0.06 | 0.15 |
| 1930 | 0.86 | 0.62 | −0.38 | −0.33 | −0.27 | −0.15 | 0.04 | 0.33 | 0.40 | −0.46 | 0.64 | −0.30 | −0.49 | −0.26 | 0.63 | 0.40 | −0.16 | −0.48 |
| 1946 | 0.57 | 0.68 | −0.22 | −0.19 | −0.29 | −0.37 | 0.23 | 0.12 | 0.34 | −0.36 | 0.74 | −0.23 | −0.43 | −0.16 | 0.73 | 0.60 | 0.10 | −0.57 |
| 1947 | −0.79 | −0.55 | 0.22 | 0.42 | 0.58 | 0.15 | 0.05 | −0.40 | −0.50 | 0.59 | −0.47 | 0.11 | 0.52 | 0.29 | −0.50 | −0.38 | −0.04 | 0.37 |
| 2002 | −0.64 | −0.25 | 0.32 | 0.40 | 0.23 | 0.13 | 0.11 | −0.29 | −0.43 | 0.26 | −0.11 | 0.12 | 0.23 | 0.08 | −0.11 | −0.36 | −0.01 | 0.21 |
| 2010 | −0.34 | −0.59 | −0.36 | 0.47 | 0.86 | −0.12 | −0.06 | −0.16 | −0.54 | 0.72 | −0.25 | −0.15 | −0.15 | 0.46 | −0.13 | −0.17 | −0.57 | 0.17 |
| 2011 | −0.35 | 0.22 | 0.12 | −0.37 | −0.08 | 0.20 | 0.42 | −0.25 | 0.10 | 0.35 | −0.13 | 0.39 | 0.38 | −0.17 | 0.05 | 0.17 | 0.21 | −0.27 |
| 2018 | 0.05 | −0.11 | −0.13 | 0.08 | 0.26 | 0.48 | −0.17 | 0.10 | −0.17 | 0.02 | 0.08 | −0.20 | −0.17 | −0.06 | 0.00 | −0.08 | −0.32 | −0.03 |
| 2035 | −0.51 | −0.43 | 0.17 | 0.51 | 0.32 | 0.09 | 0.22 | −0.20 | −0.48 | 0.27 | −0.40 | 0.01 | 0.02 | 0.36 | −0.28 | −0.24 | −0.08 | 0.28 |
| 2052 | −0.58 | −0.18 | 0.27 | 0.27 | 0.32 | −0.01 | 0.02 | −0.51 | −0.25 | 0.29 | −0.30 | 0.27 | 0.55 | 0.09 | −0.36 | −0.47 | 0.31 | 0.08 |
| 2068 | −0.73 | −0.68 | −0.15 | 0.58 | 0.65 | 0.09 | 0.23 | −0.31 | −0.54 | 0.60 | −0.51 | 0.00 | 0.01 | 0.39 | −0.32 | −0.25 | −0.25 | 0.29 |
| 2076 | 1.00 | 0.67 | −0.03 | −0.54 | −0.52 | −0.17 | −0.04 | 0.32 | 0.52 | −0.55 | 0.44 | −0.10 | −0.21 | −0.35 | 0.36 | 0.17 | 0.05 | −0.31 |
| 2092 | 0.67 | 1.00 | 0.08 | −0.55 | −0.54 | −0.06 | 0.23 | −0.14 | 0.73 | −0.51 | 0.58 | 0.16 | 0.03 | −0.28 | 0.47 | 0.38 | 0.43 | −0.72 |
| 2117 | −0.03 | 0.08 | 1.00 | −0.10 | −0.48 | −0.02 | −0.15 | −0.16 | 0.07 | −0.21 | −0.21 | 0.25 | 0.31 | −0.18 | −0.50 | −0.37 | 0.53 | 0.30 |
| 2133 | −0.54 | −0.55 | −0.10 | 1.00 | 0.47 | −0.07 | −0.02 | −0.14 | −0.61 | 0.17 | 0.00 | −0.41 | −0.04 | 0.12 | −0.05 | 0.07 | −0.28 | 0.39 |
| 2156 | −0.52 | −0.54 | −0.48 | 0.47 | 1.00 | −0.08 | 0.00 | −0.28 | −0.55 | 0.79 | −0.16 | −0.08 | 0.09 | 0.47 | −0.06 | −0.15 | −0.45 | 0.04 |
| 2157 | −0.17 | −0.06 | −0.02 | −0.07 | −0.08 | 1.00 | 0.06 | −0.28 | 0.30 | −0.01 | −0.32 | 0.41 | −0.09 | −0.20 | −0.22 | −0.05 | 0.10 | −0.23 |
| 2164 | −0.04 | 0.23 | −0.15 | −0.02 | 0.00 | 0.06 | 1.00 | −0.10 | −0.09 | 0.18 | 0.06 | −0.23 | 0.18 | −0.17 | 0.15 | 0.45 | −0.14 | −0.20 |
| 2221 | 0.32 | −0.14 | −0.16 | −0.14 | −0.28 | −0.28 | −0.10 | 1.00 | −0.34 | −0.32 | 0.21 | −0.61 | −0.28 | −0.09 | 0.29 | 0.20 | −0.54 | 0.51 |
| 2222 | 0.52 | 0.73 | 0.07 | −0.61 | −0.55 | 0.30 | −0.09 | −0.34 | 1.00 | −0.50 | 0.17 | 0.53 | −0.16 | −0.17 | 0.11 | 0.13 | 0.67 | −0.73 |
| 2230 | −0.55 | −0.51 | −0.21 | 0.17 | 0.79 | −0.01 | 0.18 | −0.32 | −0.50 | 1.00 | −0.38 | 0.23 | 0.30 | 0.27 | −0.18 | −0.20 | −0.37 | 0.08 |
| 2237 | 0.44 | 0.58 | −0.21 | 0.00 | −0.16 | −0.32 | 0.06 | 0.21 | 0.17 | −0.38 | 1.00 | −0.37 | −0.22 | −0.19 | 0.85 | 0.54 | −0.06 | −0.37 |
| 2238 | −0.10 | 0.16 | 0.25 | −0.41 | −0.08 | 0.41 | −0.23 | −0.61 | 0.53 | 0.23 | −0.37 | 1.00 | 0.24 | 0.02 | −0.24 | −0.41 | 0.52 | −0.41 |
| 2239 | −0.21 | 0.03 | 0.31 | −0.04 | 0.09 | −0.09 | 0.18 | −0.28 | −0.16 | 0.30 | −0.22 | 0.24 | 1.00 | −0.17 | −0.28 | −0.22 | 0.16 | 0.18 |
| 2246 | −0.35 | −0.28 | −0.18 | 0.12 | 0.47 | −0.20 | −0.17 | −0.09 | −0.17 | 0.27 | −0.19 | 0.02 | −0.17 | 1.00 | −0.13 | −0.08 | −0.06 | −0.01 |
| 2253 | 0.36 | 0.47 | −0.50 | −0.05 | −0.06 | −0.22 | 0.15 | 0.29 | 0.11 | −0.18 | 0.85 | −0.24 | −0.28 | −0.13 | 1.00 | 0.60 | −0.29 | −0.41 |
| 2254 | 0.17 | 0.38 | −0.37 | 0.07 | −0.15 | −0.05 | 0.45 | 0.20 | 0.13 | −0.20 | 0.54 | −0.41 | −0.22 | −0.08 | 0.60 | 1.00 | −0.08 | −0.35 |
| 2263 | 0.05 | 0.43 | 0.53 | −0.28 | −0.45 | 0.10 | −0.14 | −0.54 | 0.67 | −0.37 | −0.06 | 0.52 | 0.16 | −0.06 | −0.29 | −0.08 | 1.00 | −0.38 |
| 2279 | −0.31 | −0.72 | 0.30 | 0.39 | 0.04 | −0.23 | −0.20 | 0.51 | −0.73 | 0.08 | −0.37 | −0.41 | 0.18 | −0.01 | −0.41 | −0.35 | −0.38 | 1.00 |
| 2280 | −0.27 | −0.59 | 0.12 | 0.57 | −0.04 | −0.15 | −0.17 | 0.47 | −0.60 | −0.23 | −0.18 | −0.55 | −0.18 | 0.15 | −0.20 | −0.14 | −0.26 | 0.78 |
| 2295 | 0.36 | 0.53 | −0.07 | −0.06 | 0.01 | −0.41 | 0.46 | −0.14 | 0.12 | −0.11 | 0.62 | −0.30 | 0.18 | −0.31 | 0.37 | 0.22 | 0.05 | −0.34 |
| 2321 | −0.33 | 0.15 | 0.11 | −0.15 | −0.13 | 0.12 | 0.30 | −0.24 | 0.06 | 0.20 | −0.05 | 0.16 | 0.21 | −0.13 | −0.03 | 0.30 | 0.20 | −0.22 |
| 2367 | 0.73 | 0.42 | −0.03 | −0.63 | −0.71 | 0.12 | −0.21 | 0.56 | 0.55 | −0.68 | 0.19 | −0.04 | −0.38 | −0.25 | 0.19 | 0.15 | 0.04 | −0.11 |
| 2368 | 0.42 | 0.52 | 0.15 | −0.17 | −0.33 | 0.40 | −0.02 | −0.25 | 0.66 | −0.45 | 0.30 | 0.11 | −0.29 | −0.06 | 0.06 | 0.32 | 0.35 | −0.51 |
| 2383 | 0.47 | 0.57 | −0.03 | −0.09 | −0.32 | −0.25 | 0.41 | −0.01 | 0.32 | −0.36 | 0.40 | −0.34 | −0.24 | −0.14 | 0.28 | 0.69 | 0.27 | −0.43 |
| 2384 | 0.16 | 0.60 | −0.12 | −0.36 | −0.26 | −0.15 | 0.12 | −0.03 | 0.30 | −0.12 | 0.38 | 0.15 | −0.03 | −0.15 | 0.52 | 0.23 | 0.05 | −0.48 |
| 2390 | 0.47 | 0.58 | −0.24 | −0.36 | −0.31 | 0.05 | 0.17 | −0.21 | 0.72 | −0.42 | 0.35 | 0.02 | −0.36 | −0.17 | 0.26 | 0.36 | 0.48 | −0.65 |
| 2400 | −0.26 | 0.07 | 0.54 | 0.00 | −0.16 | −0.12 | −0.24 | −0.16 | −0.01 | −0.06 | 0.15 | 0.12 | 0.46 | −0.09 | −0.19 | −0.12 | 0.34 | 0.15 |
| 2408 | 0.43 | 0.49 | 0.19 | −0.51 | −0.50 | 0.33 | −0.10 | −0.35 | 0.82 | −0.31 | −0.06 | 0.55 | −0.08 | −0.16 | −0.12 | 0.11 | 0.64 | −0.54 |
| 2425 | −0.40 | 0.01 | 0.69 | 0.08 | −0.13 | 0.35 | −0.18 | −0.64 | 0.24 | 0.09 | −0.35 | 0.63 | 0.29 | 0.03 | −0.47 | −0.25 | 0.59 | −0.06 |
| 2441 | 0.56 | 0.35 | 0.22 | −0.60 | −0.75 | 0.02 | −0.29 | 0.28 | 0.64 | −0.62 | −0.07 | 0.22 | −0.07 | −0.27 | −0.13 | −0.03 | 0.43 | −0.03 |
| 2447 | −0.27 | −0.34 | −0.37 | 0.16 | 0.78 | 0.02 | 0.40 | −0.21 | −0.44 | 0.72 | −0.31 | −0.06 | 0.25 | 0.31 | −0.20 | −0.20 | −0.49 | 0.00 |
| 2448 | 0.06 | −0.29 | −0.41 | 0.32 | 0.61 | 0.05 | 0.01 | −0.01 | −0.41 | 0.45 | 0.02 | −0.26 | −0.05 | −0.16 | 0.03 | −0.08 | −0.63 | 0.03 |
| 2482 | −0.75 | −0.54 | 0.41 | 0.55 | 0.24 | −0.04 | −0.18 | −0.35 | −0.39 | 0.35 | −0.45 | 0.17 | 0.01 | 0.23 | −0.46 | −0.29 | 0.14 | 0.36 |
| 2512 | 0.19 | −0.02 | −0.26 | 0.20 | 0.15 | −0.28 | 0.13 | 0.17 | −0.26 | 0.16 | 0.47 | −0.19 | 0.09 | −0.09 | 0.56 | 0.21 | −0.35 | 0.08 |
| 2513 | 0.19 | 0.05 | 0.04 | 0.13 | 0.01 | −0.20 | 0.17 | −0.05 | −0.13 | 0.02 | −0.02 | −0.17 | 0.46 | −0.06 | −0.01 | −0.08 | −0.13 | 0.22 |
| 2521 | −0.44 | 0.04 | −0.37 | 0.15 | 0.36 | −0.02 | 0.36 | −0.29 | −0.16 | 0.31 | −0.16 | 0.10 | 0.25 | 0.28 | 0.09 | 0.09 | −0.10 | −0.26 |
| 2522 | −0.12 | −0.30 | −0.36 | 0.18 | 0.62 | 0.12 | −0.20 | −0.10 | −0.31 | 0.53 | −0.16 | 0.03 | 0.29 | −0.15 | −0.09 | −0.13 | −0.40 | 0.02 |
| 2528 | 0.24 | 0.38 | −0.34 | 0.11 | −0.14 | −0.12 | 0.49 | 0.18 | 0.07 | −0.18 | 0.52 | −0.45 | −0.04 | −0.10 | 0.58 | 0.93 | −0.13 | −0.26 |
| 2529 | 0.29 | 0.12 | 0.15 | 0.12 | −0.08 | −0.31 | 0.24 | 0.09 | −0.21 | 0.04 | 0.24 | −0.14 | −0.18 | −0.10 | 0.23 | 0.07 | −0.23 | 0.00 |
| 2544 | 0.17 | 0.38 | −0.37 | 0.07 | −0.15 | −0.05 | 0.44 | 0.20 | 0.12 | −0.19 | 0.55 | −0.41 | −0.22 | −0.08 | 0.61 | 1.00 | −0.08 | −0.35 |
| 2570 | 0.04 | −0.40 | 0.15 | 0.09 | −0.15 | −0.07 | −0.05 | 0.69 | −0.47 | −0.12 | −0.07 | −0.63 | −0.21 | −0.22 | −0.18 | −0.02 | −0.49 | 0.70 |
| 2571 | −0.21 | −0.45 | 0.18 | −0.13 | 0.04 | −0.26 | −0.45 | 0.53 | −0.40 | 0.11 | −0.25 | −0.18 | 0.04 | 0.41 | −0.30 | −0.22 | −0.23 | 0.59 |
| 2586 | −0.01 | 0.09 | −0.05 | −0.20 | 0.12 | 0.02 | 0.47 | 0.03 | −0.12 | 0.29 | 0.09 | 0.15 | 0.39 | −0.09 | 0.20 | 0.07 | −0.07 | −0.09 |
| 2587 | 0.38 | 0.55 | 0.13 | −0.49 | −0.42 | 0.41 | −0.17 | −0.41 | 0.92 | −0.34 | 0.06 | 0.56 | −0.25 | −0.09 | 0.01 | 0.11 | 0.60 | −0.65 |
| 2603 | −0.27 | 0.04 | 0.19 | −0.28 | −0.05 | 0.05 | −0.16 | −0.19 | 0.03 | 0.32 | −0.11 | 0.47 | 0.40 | −0.07 | −0.03 | −0.04 | 0.22 | −0.10 |
| 2644 | −0.35 | −0.76 | 0.10 | 0.20 | 0.09 | −0.17 | −0.27 | 0.60 | −0.66 | 0.16 | −0.47 | −0.33 | 0.05 | 0.05 | −0.41 | −0.31 | −0.41 | 0.90 |
| 2645 | −0.02 | −0.44 | 0.01 | −0.05 | −0.25 | −0.13 | −0.18 | 0.79 | −0.39 | −0.25 | −0.26 | −0.43 | 0.00 | −0.07 | −0.23 | −0.17 | −0.32 | 0.74 |
| 2660 | 0.36 | 0.29 | 0.10 | −0.30 | −0.16 | −0.20 | −0.17 | −0.22 | 0.46 | −0.27 | 0.10 | 0.08 | −0.17 | −0.06 | −0.13 | −0.08 | 0.62 | −0.33 |
| 2683 | −0.56 | −0.34 | −0.02 | 0.28 | 0.39 | −0.01 | 0.16 | −0.40 | −0.33 | 0.50 | −0.58 | 0.28 | 0.57 | 0.11 | −0.42 | −0.28 | 0.02 | 0.13 |
| 2714 | −0.37 | −0.28 | −0.01 | 0.57 | −0.04 | 0.14 | −0.17 | −0.08 | −0.17 | −0.27 | −0.19 | −0.03 | 0.04 | −0.06 | −0.13 | −0.08 | 0.17 | 0.24 |
| 2732 | 0.54 | 0.03 | −0.22 | −0.49 | −0.46 | −0.08 | −0.14 | 0.70 | 0.22 | −0.44 | −0.04 | −0.24 | −0.43 | −0.04 | 0.04 | 0.08 | −0.18 | 0.17 |
| 2733 | 0.11 | 0.15 | 0.08 | 0.01 | −0.05 | 0.48 | −0.24 | −0.25 | 0.33 | −0.31 | −0.05 | 0.33 | 0.10 | −0.08 | −0.17 | −0.32 | 0.24 | −0.21 |
| 2807 | 0.28 | −0.01 | 0.18 | −0.35 | −0.46 | 0.07 | −0.60 | 0.30 | 0.43 | −0.46 | −0.17 | 0.19 | −0.20 | 0.08 | −0.25 | −0.07 | 0.30 | 0.14 |
| 2878 | 0.15 | 0.24 | −0.35 | 0.08 | −0.02 | −0.07 | −0.24 | 0.17 | 0.04 | −0.27 | 0.53 | −0.02 | −0.24 | −0.12 | 0.68 | 0.00 | −0.18 | −0.22 |
| 2879 | 0.14 | 0.26 | −0.11 | 0.26 | −0.10 | −0.28 | 0.25 | 0.20 | −0.08 | −0.20 | 0.76 | −0.46 | 0.03 | −0.13 | 0.59 | 0.70 | −0.10 | −0.06 |
| 2880 | 0.28 | 0.18 | 0.04 | −0.30 | −0.36 | 0.61 | −0.17 | −0.21 | 0.65 | −0.27 | −0.19 | 0.56 | −0.17 | −0.06 | −0.13 | −0.08 | 0.25 | −0.29 |
| 2886 | −0.67 | −0.50 | −0.31 | 0.41 | 0.58 | 0.05 | 0.16 | −0.28 | −0.34 | 0.39 | −0.49 | 0.07 | 0.15 | 0.51 | −0.33 | −0.20 | −0.07 | 0.12 |
| 2936 | 0.23 | −0.31 | −0.07 | −0.26 | −0.26 | −0.07 | −0.21 | 0.89 | −0.29 | −0.24 | −0.12 | −0.44 | −0.23 | −0.08 | −0.08 | −0.08 | −0.43 | 0.58 |
| 2953 | 0.46 | 0.55 | −0.11 | −0.33 | −0.45 | 0.27 | 0.12 | −0.15 | 0.79 | −0.49 | 0.20 | 0.11 | −0.31 | −0.11 | 0.13 | 0.57 | 0.51 | −0.60 |
| 3024 | −0.20 | −0.18 | −0.30 | 0.15 | 0.48 | 0.18 | −0.13 | 0.04 | −0.19 | 0.15 | 0.00 | −0.21 | −0.28 | 0.71 | 0.00 | 0.12 | −0.26 | −0.12 |
| 3025 | 0.14 | 0.25 | −0.30 | 0.07 | 0.01 | 0.35 | 0.23 | 0.17 | 0.05 | −0.19 | 0.34 | −0.43 | −0.24 | −0.09 | 0.30 | 0.66 | −0.19 | −0.29 |
| 3098 | 0.27 | −0.19 | −0.06 | −0.35 | −0.37 | −0.22 | −0.21 | 0.89 | −0.18 | −0.31 | −0.09 | −0.37 | −0.21 | −0.08 | −0.01 | −0.09 | −0.35 | 0.53 |
| 3099 | 0.46 | 0.49 | −0.28 | −0.30 | −0.34 | 0.35 | 0.04 | −0.14 | 0.77 | −0.37 | 0.16 | 0.23 | −0.29 | −0.14 | 0.21 | 0.50 | 0.27 | −0.60 |
| 3170 | −0.11 | 0.05 | 0.47 | 0.21 | −0.13 | −0.20 | −0.17 | −0.04 | −0.05 | −0.27 | 0.31 | −0.19 | 0.24 | −0.06 | −0.13 | −0.08 | 0.21 | 0.24 |
| 3171 | 0.10 | −0.06 | −0.32 | 0.15 | 0.16 | −0.20 | 0.04 | 0.22 | −0.22 | 0.17 | 0.54 | −0.12 | −0.17 | −0.06 | 0.64 | 0.29 | −0.32 | −0.04 |
| 3172 | 0.32 | 0.32 | −0.06 | −0.28 | −0.42 | 0.50 | 0.04 | 0.15 | 0.70 | 0.05 | 0.05 | 0.00 | 0.22 | 0.00 | 0.02 | 0.27 | 0.25 | 0.44 |
| 3390 | 0.15 | 0.33 | −0.31 | 0.05 | −0.07 | 0.25 | 0.32 | 0.17 | 0.11 | −0.23 | 0.37 | −0.44 | −0.23 | −0.09 | 0.36 | 0.81 | −0.12 | −0.34 |
| 3463 | 0.34 | 0.66 | −0.32 | −0.26 | −0.26 | −0.16 | 0.10 | 0.15 | 0.38 | −0.38 | 0.57 | −0.11 | −0.24 | −0.09 | 0.69 | 0.44 | −0.02 | −0.50 |
| | 2280 | 2295 | 2321 | 2367 | 2368 | 2383 | 2384 | 2390 | 2400 | 2408 | 2425 | 2441 | 2447 | 2448 | 2482 | 2512 | 2513 | 2521 |
| 1354 | 0.03 | −0.09 | −0.02 | −0.45 | −0.18 | −0.27 | −0.20 | −0.12 | −0.21 | −0.34 | −0.18 | −0.53 | 0.70 | 0.46 | 0.19 | −0.09 | 0.09 | 0.34 |
| 1362 | 0.19 | 0.23 | −0.19 | −0.17 | −0.19 | 0.08 | −0.10 | −0.01 | −0.13 | −0.08 | −0.30 | −0.18 | 0.09 | 0.17 | −0.31 | 0.86 | 0.85 | −0.17 |
| 1403 | 0.04 | 0.19 | −0.13 | −0.10 | −0.17 | 0.03 | −0.08 | −0.02 | −0.09 | −0.10 | −0.38 | −0.23 | 0.01 | 0.20 | −0.22 | 0.86 | −0.06 | −0.22 |
| 1475 | 0.46 | −0.50 | 0.08 | −0.59 | −0.33 | −0.34 | −0.18 | −0.44 | 0.19 | −0.28 | 0.58 | −0.33 | −0.14 | −0.21 | 0.91 | −0.17 | −0.10 | 0.20 |
| 1500 | −0.01 | −0.13 | 0.19 | −0.75 | −0.47 | −0.43 | −0.09 | −0.32 | 0.11 | −0.36 | 0.28 | −0.52 | 0.52 | −0.04 | 0.61 | −0.21 | −0.15 | 0.57 |
| 1516 | 0.22 | −0.07 | 0.05 | −0.42 | −0.16 | −0.07 | −0.10 | −0.11 | −0.22 | −0.25 | −0.15 | −0.59 | 0.52 | 0.18 | 0.23 | 0.13 | 0.09 | 0.28 |
| 1541 | −0.05 | 0.04 | 0.00 | −0.47 | −0.29 | −0.23 | −0.17 | −0.10 | −0.20 | −0.32 | −0.24 | −0.43 | 0.67 | 0.45 | 0.21 | 0.08 | 0.07 | 0.33 |
| 1549 | −0.14 | 0.32 | −0.16 | 0.10 | 0.11 | 0.45 | −0.17 | 0.72 | −0.11 | 0.43 | −0.19 | 0.27 | −0.21 | −0.11 | −0.27 | 0.13 | −0.08 | −0.36 |
| 1557 | −0.17 | 0.07 | −0.23 | −0.16 | −0.05 | −0.17 | −0.24 | −0.21 | −0.16 | −0.26 | −0.35 | −0.34 | 0.49 | 0.90 | −0.16 | 0.21 | −0.11 | −0.06 |
| 1565 | 0.02 | 0.37 | −0.33 | −0.37 | −0.20 | 0.27 | −0.02 | 0.16 | −0.52 | −0.24 | −0.40 | −0.48 | 0.53 | 0.29 | −0.25 | 0.20 | 0.18 | 0.50 |
| 1637 | 0.37 | −0.42 | 0.01 | −0.63 | −0.33 | −0.39 | −0.25 | −0.48 | 0.16 | −0.36 | 0.61 | −0.36 | 0.01 | −0.25 | 0.88 | −0.21 | −0.13 | 0.21 |
| 1678 | 0.32 | −0.10 | 0.06 | −0.82 | −0.30 | −0.23 | −0.16 | −0.42 | 0.09 | −0.48 | 0.42 | −0.75 | 0.31 | 0.17 | 0.70 | −0.18 | −0.01 | 0.39 |
| 1703 | 0.00 | −0.19 | 0.36 | −0.70 | −0.60 | −0.37 | −0.02 | −0.39 | 0.04 | −0.24 | 0.02 | −0.58 | 0.53 | 0.32 | 0.34 | 0.15 | −0.07 | 0.60 |
| 1711 | 0.42 | −0.42 | −0.17 | 0.09 | −0.20 | −0.13 | −0.30 | −0.36 | 0.09 | 0.15 | 0.37 | 0.34 | −0.31 | −0.43 | 0.21 | −0.03 | 0.23 | −0.24 |
| 1719 | −0.26 | −0.08 | 0.30 | −0.57 | −0.38 | −0.32 | −0.03 | −0.37 | 0.04 | −0.21 | −0.06 | −0.62 | 0.74 | 0.51 | 0.08 | 0.16 | 0.02 | 0.41 |
| 1727 | 0.35 | 0.33 | −0.34 | −0.49 | 0.02 | 0.10 | −0.28 | −0.04 | 0.00 | −0.43 | 0.01 | −0.53 | 0.14 | 0.26 | 0.21 | 0.04 | 0.00 | 0.10 |
| 1744 | 0.25 | −0.12 | 0.07 | −0.46 | −0.19 | −0.06 | −0.09 | −0.14 | −0.23 | −0.26 | 0.16 | −0.51 | 0.26 | 0.03 | 0.57 | 0.08 | 0.05 | 0.05 |
| 1768 | 0.17 | 0.23 | 0.10 | −0.27 | −0.09 | 0.07 | −0.02 | −0.02 | −0.22 | −0.19 | −0.30 | −0.57 | 0.38 | 0.56 | −0.05 | 0.38 | 0.25 | −0.01 |
| 1791 | 0.00 | −0.12 | 0.01 | −0.43 | −0.22 | −0.24 | −0.16 | −0.11 | −0.17 | −0.29 | −0.13 | −0.43 | 0.60 | 0.23 | 0.30 | −0.08 | −0.12 | 0.38 |
| 1799 | 0.43 | −0.31 | −0.15 | −0.48 | −0.36 | −0.27 | −0.26 | −0.24 | −0.21 | −0.35 | 0.25 | −0.27 | −0.02 | 0.08 | 0.65 | −0.03 | 0.04 | 0.12 |
| 1840 | 0.30 | −0.10 | −0.23 | −0.34 | −0.29 | −0.26 | 0.06 | −0.26 | −0.32 | −0.45 | 0.22 | −0.40 | 0.06 | −0.02 | 0.38 | −0.11 | 0.00 | 0.29 |
| 1865 | −0.05 | 0.04 | −0.08 | −0.63 | −0.42 | −0.29 | −0.21 | −0.31 | −0.16 | −0.43 | −0.26 | −0.68 | 0.68 | 0.62 | 0.16 | 0.43 | −0.05 | 0.29 |
| 1873 | 0.15 | 0.11 | 0.05 | −0.68 | −0.28 | −0.28 | 0.07 | −0.41 | 0.18 | −0.45 | 0.30 | −0.71 | 0.23 | 0.40 | 0.59 | 0.02 | 0.00 | 0.17 |
| 1889 | 0.02 | 0.21 | 0.08 | −0.64 | −0.22 | −0.05 | 0.15 | −0.19 | 0.04 | −0.39 | 0.03 | −0.80 | 0.32 | 0.54 | 0.27 | 0.10 | −0.03 | 0.33 |
| 1906 | 0.25 | 0.06 | 0.02 | −0.92 | −0.41 | −0.29 | −0.38 | −0.45 | 0.13 | −0.51 | 0.28 | −0.72 | 0.54 | 0.16 | 0.56 | 0.19 | 0.24 | 0.30 |
| 1914 | 0.09 | 0.26 | −0.05 | −0.77 | −0.37 | −0.14 | −0.09 | −0.40 | 0.23 | −0.34 | 0.28 | −0.61 | 0.33 | 0.14 | 0.47 | 0.29 | 0.16 | 0.17 |
| 1930 | −0.25 | 0.41 | −0.26 | 0.58 | 0.38 | 0.56 | 0.30 | 0.55 | −0.41 | 0.24 | −0.59 | 0.24 | −0.16 | 0.25 | −0.71 | 0.23 | 0.00 | −0.22 |
| 1946 | −0.27 | 0.51 | 0.06 | 0.25 | 0.23 | 0.76 | 0.48 | 0.56 | −0.23 | 0.22 | −0.37 | 0.06 | −0.29 | −0.05 | −0.36 | 0.28 | −0.16 | 0.00 |
| 1947 | 0.15 | −0.16 | 0.05 | −0.73 | −0.29 | −0.54 | −0.33 | −0.52 | 0.27 | −0.49 | 0.46 | −0.56 | 0.47 | 0.06 | 0.54 | −0.17 | 0.16 | 0.28 |
| 2002 | 0.26 | −0.05 | 0.12 | −0.63 | −0.35 | −0.45 | 0.13 | −0.49 | 0.23 | −0.52 | 0.41 | −0.64 | 0.15 | −0.10 | 0.57 | −0.06 | −0.02 | 0.40 |
| 2010 | 0.08 | −0.13 | −0.21 | −0.52 | −0.19 | −0.22 | −0.31 | −0.32 | −0.30 | −0.41 | −0.11 | −0.63 | 0.66 | 0.66 | 0.32 | 0.15 | 0.12 | 0.10 |
| 2011 | −0.46 | 0.00 | 0.67 | −0.20 | −0.20 | −0.05 | 0.51 | −0.02 | 0.20 | 0.03 | 0.33 | −0.12 | 0.02 | −0.33 | 0.15 | −0.24 | −0.17 | 0.45 |
| 2018 | 0.08 | −0.13 | −0.13 | 0.03 | 0.21 | −0.14 | −0.15 | −0.11 | −0.09 | −0.16 | −0.13 | −0.35 | 0.22 | 0.53 | −0.22 | −0.09 | −0.06 | −0.12 |
| 2035 | 0.48 | −0.27 | −0.23 | −0.59 | −0.35 | −0.16 | −0.30 | −0.44 | −0.33 | −0.41 | 0.23 | −0.56 | 0.36 | −0.04 | 0.53 | −0.03 | 0.02 | 0.39 |
| 2052 | 0.06 | 0.07 | 0.07 | −0.70 | −0.45 | −0.34 | −0.02 | −0.31 | 0.26 | −0.31 | 0.37 | −0.43 | 0.28 | −0.09 | 0.45 | −0.22 | −0.01 | 0.54 |
| 2068 | 0.33 | −0.23 | −0.15 | −0.72 | −0.43 | −0.35 | −0.35 | −0.34 | −0.35 | −0.52 | 0.13 | −0.63 | 0.56 | 0.14 | 0.58 | −0.03 | 0.05 | 0.40 |
| 2076 | −0.27 | 0.36 | −0.33 | 0.73 | 0.42 | 0.47 | 0.16 | 0.47 | −0.26 | 0.43 | −0.40 | 0.56 | −0.27 | 0.06 | −0.75 | 0.19 | 0.19 | −0.44 |
| 2092 | −0.59 | 0.53 | 0.15 | 0.42 | 0.52 | 0.57 | 0.60 | 0.58 | 0.07 | 0.49 | 0.01 | 0.35 | −0.34 | −0.29 | −0.54 | −0.02 | 0.05 | 0.04 |
| 2117 | 0.12 | −0.07 | 0.11 | −0.03 | 0.15 | −0.03 | −0.12 | −0.24 | 0.54 | 0.19 | 0.69 | 0.22 | −0.37 | −0.41 | 0.41 | −0.26 | 0.04 | −0.37 |
| 2133 | 0.57 | −0.06 | −0.15 | −0.63 | −0.17 | −0.09 | −0.36 | −0.36 | 0.00 | −0.51 | 0.08 | −0.60 | 0.16 | 0.32 | 0.55 | 0.20 | 0.13 | 0.15 |
| 2156 | −0.04 | 0.01 | −0.13 | −0.71 | −0.33 | −0.32 | −0.26 | −0.31 | −0.16 | −0.50 | −0.13 | −0.75 | 0.78 | 0.61 | 0.24 | 0.15 | 0.01 | 0.36 |
| 2157 | −0.15 | −0.41 | 0.12 | 0.12 | 0.40 | −0.25 | −0.15 | 0.05 | −0.12 | 0.33 | 0.35 | 0.02 | 0.02 | 0.05 | −0.04 | −0.28 | −0.20 | −0.02 |
| 2164 | −0.17 | 0.46 | 0.30 | −0.21 | −0.02 | 0.41 | 0.12 | 0.17 | −0.24 | −0.10 | −0.18 | −0.29 | 0.40 | 0.01 | −0.18 | 0.13 | 0.17 | 0.36 |
| 2221 | 0.47 | −0.14 | −0.24 | 0.56 | −0.25 | −0.01 | −0.03 | −0.21 | −0.16 | −0.35 | −0.64 | 0.28 | −0.21 | −0.01 | −0.35 | 0.17 | −0.05 | −0.29 |
| 2222 | −0.60 | 0.12 | 0.06 | 0.55 | 0.66 | 0.32 | 0.30 | 0.72 | −0.01 | 0.82 | 0.24 | 0.64 | −0.44 | −0.41 | −0.39 | −0.26 | −0.13 | −0.16 |
| 2230 | −0.23 | −0.11 | 0.20 | −0.68 | −0.45 | −0.36 | −0.12 | −0.42 | −0.06 | −0.31 | 0.09 | −0.62 | 0.72 | 0.45 | 0.35 | 0.16 | 0.02 | 0.31 |
| 2237 | −0.18 | 0.62 | −0.05 | 0.19 | 0.30 | 0.40 | 0.38 | 0.35 | 0.15 | −0.06 | −0.35 | −0.07 | −0.31 | 0.02 | −0.45 | 0.47 | −0.02 | −0.16 |
| 2238 | −0.55 | −0.30 | 0.16 | −0.04 | 0.11 | −0.34 | 0.15 | 0.02 | 0.12 | 0.55 | 0.63 | 0.22 | −0.06 | −0.26 | 0.17 | −0.19 | −0.17 | 0.10 |
| 2239 | −0.18 | 0.18 | 0.21 | −0.38 | −0.29 | −0.24 | −0.03 | −0.36 | 0.46 | −0.08 | 0.29 | −0.07 | 0.25 | −0.05 | 0.01 | 0.09 | 0.46 | 0.25 |
| 2246 | 0.15 | −0.31 | −0.13 | −0.25 | −0.06 | −0.14 | −0.15 | −0.17 | −0.09 | −0.16 | 0.03 | −0.27 | 0.31 | −0.16 | 0.23 | −0.09 | −0.06 | 0.28 |
| 2253 | −0.20 | 0.37 | −0.03 | 0.19 | 0.06 | 0.28 | 0.52 | 0.26 | −0.19 | −0.12 | −0.47 | −0.13 | −0.20 | 0.03 | −0.46 | 0.56 | −0.01 | 0.09 |
| 2254 | −0.14 | 0.22 | 0.30 | 0.15 | 0.32 | 0.69 | 0.23 | 0.36 | −0.12 | 0.11 | −0.25 | −0.03 | −0.20 | −0.08 | −0.29 | 0.21 | −0.08 | 0.09 |
| 2263 | −0.26 | 0.05 | 0.20 | 0.04 | 0.35 | 0.27 | 0.05 | 0.48 | 0.34 | 0.64 | 0.59 | 0.43 | −0.49 | −0.63 | 0.14 | −0.35 | −0.13 | −0.10 |
| 2279 | 0.78 | −0.34 | −0.22 | −0.11 | −0.51 | −0.43 | −0.48 | −0.65 | 0.15 | −0.54 | −0.06 | −0.03 | 0.00 | 0.03 | 0.36 | 0.08 | 0.22 | −0.26 |
| 2280 | 1.00 | −0.38 | −0.29 | −0.09 | −0.39 | −0.14 | −0.42 | −0.35 | −0.11 | −0.45 | −0.18 | −0.13 | −0.17 | −0.09 | 0.31 | 0.14 | 0.20 | −0.15 |
| 2295 | −0.38 | 1.00 | −0.05 | −0.10 | 0.16 | 0.40 | 0.19 | 0.38 | 0.14 | −0.12 | −0.29 | −0.12 | 0.19 | 0.16 | −0.39 | 0.24 | 0.15 | 0.01 |
| 2321 | −0.29 | −0.05 | 1.00 | −0.18 | −0.04 | 0.21 | 0.52 | 0.06 | 0.46 | 0.27 | 0.29 | −0.11 | −0.17 | −0.15 | 0.26 | −0.19 | −0.13 | 0.31 |
| 2367 | −0.09 | −0.10 | −0.18 | 1.00 | 0.41 | 0.16 | 0.15 | 0.34 | −0.13 | 0.42 | −0.29 | 0.82 | −0.50 | −0.25 | −0.61 | −0.15 | −0.13 | −0.46 |
| 2368 | −0.39 | 0.16 | −0.04 | 0.41 | 1.00 | 0.38 | −0.05 | 0.45 | 0.12 | 0.56 | 0.31 | 0.30 | −0.26 | −0.07 | −0.28 | −0.21 | −0.11 | −0.40 |
| 2383 | −0.14 | 0.40 | 0.21 | 0.16 | 0.38 | 1.00 | 0.17 | 0.63 | −0.19 | 0.40 | −0.19 | 0.12 | −0.23 | −0.07 | −0.27 | 0.06 | 0.07 | −0.06 |
| 2384 | −0.42 | 0.19 | 0.52 | 0.15 | −0.05 | 0.17 | 1.00 | 0.18 | 0.11 | 0.08 | −0.01 | 0.00 | −0.29 | −0.19 | −0.08 | −0.10 | −0.06 | 0.46 |
| 2390 | −0.35 | 0.38 | 0.06 | 0.34 | 0.45 | 0.63 | 0.18 | 1.00 | −0.24 | 0.58 | −0.20 | 0.37 | −0.31 | −0.22 | −0.46 | −0.02 | 0.00 | −0.17 |
| 2400 | −0.11 | 0.14 | 0.46 | −0.13 | 0.12 | −0.19 | 0.11 | −0.24 | 1.00 | 0.05 | 0.48 | 0.07 | −0.30 | −0.23 | 0.27 | −0.13 | −0.09 | −0.15 |
| 2408 | −0.45 | −0.12 | 0.27 | 0.42 | 0.56 | 0.40 | 0.08 | 0.58 | 0.05 | 1.00 | 0.31 | 0.59 | −0.41 | −0.27 | −0.24 | −0.10 | −0.03 | −0.30 |
| 2425 | −0.18 | −0.29 | 0.29 | −0.29 | 0.31 | −0.19 | −0.01 | −0.20 | 0.48 | 0.31 | 1.00 | 0.00 | −0.23 | −0.35 | 0.63 | −0.40 | −0.11 | −0.01 |
| 2441 | −0.13 | −0.12 | −0.11 | 0.82 | 0.30 | 0.12 | 0.00 | 0.37 | 0.07 | 0.59 | 0.00 | 1.00 | −0.58 | −0.48 | −0.38 | −0.23 | −0.05 | −0.45 |
| 2447 | −0.17 | 0.19 | −0.17 | −0.50 | −0.26 | −0.23 | −0.29 | −0.31 | −0.30 | −0.41 | −0.23 | −0.58 | 1.00 | 0.50 | −0.04 | 0.05 | 0.09 | 0.40 |
| 2448 | −0.09 | 0.16 | −0.15 | −0.25 | −0.07 | −0.07 | −0.19 | −0.22 | −0.23 | −0.27 | −0.35 | −0.48 | 0.60 | 1.00 | −0.10 | 0.21 | 0.08 | 0.00 |
| 2482 | 0.31 | −0.39 | 0.26 | −0.61 | −0.28 | −0.27 | −0.08 | −0.46 | 0.27 | −0.24 | 0.63 | −0.38 | −0.04 | −0.10 | 1.00 | −0.31 | −0.22 | 0.20 |
| 2512 | 0.14 | 0.24 | −0.19 | −0.15 | −0.21 | 0.06 | −0.10 | −0.02 | −0.13 | −0.10 | −0.40 | −0.23 | 0.05 | 0.21 | −0.31 | 1.00 | 0.45 | −0.23 |
| 2513 | 0.20 | 0.15 | −0.13 | −0.13 | −0.11 | 0.07 | −0.06 | 0.00 | −0.09 | −0.03 | −0.11 | −0.05 | 0.09 | 0.08 | −0.22 | 0.46 | 1.00 | −0.06 |
| 2521 | −0.15 | 0.01 | 0.31 | −0.46 | −0.40 | −0.06 | 0.46 | −0.17 | −0.15 | −0.30 | −0.01 | −0.46 | 0.40 | 0.00 | 0.20 | −0.23 | −0.06 | 1.00 |
| 2522 | −0.25 | −0.04 | −0.03 | −0.28 | −0.19 | −0.27 | −0.15 | −0.32 | −0.02 | −0.20 | −0.13 | −0.31 | 0.52 | 0.80 | −0.04 | 0.01 | −0.03 | 0.15 |
| 2528 | −0.06 | 0.27 | 0.24 | 0.09 | 0.26 | 0.68 | 0.20 | 0.35 | −0.15 | 0.09 | −0.28 | −0.05 | −0.16 | −0.05 | −0.36 | 0.39 | 0.29 | 0.06 |
| 2529 | 0.09 | 0.23 | −0.01 | −0.10 | −0.06 | 0.35 | 0.07 | −0.16 | −0.14 | 0.03 | −0.15 | −0.24 | 0.09 | 0.29 | 0.04 | 0.45 | 0.05 | −0.09 |
| 2544 | −0.14 | 0.22 | 0.30 | 0.14 | 0.32 | 0.68 | 0.23 | 0.36 | −0.12 | 0.11 | −0.25 | −0.03 | −0.20 | −0.08 | −0.29 | 0.23 | −0.08 | 0.08 |
| 2570 | 0.49 | −0.12 | −0.07 | 0.30 | −0.08 | −0.10 | −0.26 | −0.28 | 0.08 | −0.39 | −0.27 | 0.10 | −0.09 | 0.18 | 0.02 | −0.10 | 0.01 | −0.49 |
| 2571 | 0.35 | −0.47 | 0.11 | 0.16 | −0.29 | −0.35 | −0.15 | −0.49 | 0.38 | −0.22 | −0.05 | 0.14 | −0.08 | −0.11 | 0.18 | −0.15 | −0.11 | −0.21 |
| 2586 | −0.24 | 0.31 | −0.19 | −0.18 | −0.22 | −0.11 | −0.18 | −0.16 | −0.12 | −0.19 | −0.15 | −0.16 | 0.43 | −0.04 | −0.31 | 0.34 | −0.09 | 0.15 |
| 2587 | −0.59 | −0.04 | 0.08 | 0.47 | 0.76 | 0.23 | 0.18 | 0.62 | 0.02 | 0.80 | 0.42 | 0.55 | −0.42 | −0.33 | −0.20 | −0.30 | −0.16 | −0.29 |
| 2603 | −0.28 | −0.28 | 0.75 | −0.17 | −0.27 | −0.13 | 0.40 | −0.17 | 0.51 | 0.30 | 0.33 | −0.02 | −0.21 | −0.13 | 0.20 | 0.05 | −0.07 | 0.16 |
| 2644 | 0.63 | −0.47 | −0.14 | 0.02 | −0.58 | −0.47 | −0.39 | −0.60 | 0.05 | −0.51 | −0.17 | 0.08 | 0.04 | 0.04 | 0.32 | −0.13 | −0.08 | −0.15 |
| 2645 | 0.63 | −0.33 | −0.15 | 0.39 | −0.44 | −0.29 | −0.27 | −0.33 | 0.02 | −0.32 | −0.42 | 0.36 | −0.15 | −0.12 | −0.10 | −0.09 | −0.04 | −0.18 |
| 2660 | −0.15 | 0.28 | −0.13 | 0.12 | 0.15 | 0.45 | −0.15 | 0.73 | −0.09 | 0.46 | −0.10 | 0.33 | −0.21 | −0.16 | −0.22 | −0.09 | −0.06 | −0.31 |
| 2683 | 0.02 | −0.18 | 0.28 | −0.62 | −0.57 | −0.24 | 0.00 | −0.43 | 0.04 | −0.15 | 0.22 | −0.33 | 0.44 | 0.14 | 0.44 | −0.16 | 0.07 | 0.70 |
| 2714 | 0.55 | −0.31 | −0.13 | −0.25 | −0.30 | −0.14 | −0.15 | −0.17 | −0.09 | −0.16 | 0.12 | −0.08 | −0.21 | −0.15 | 0.36 | −0.09 | −0.06 | 0.35 |
| 2732 | 0.17 | −0.21 | −0.19 | 0.84 | 0.06 | 0.12 | −0.06 | 0.26 | −0.30 | 0.24 | −0.55 | 0.71 | −0.28 | −0.15 | −0.49 | −0.05 | −0.06 | −0.40 |
| 2733 | −0.15 | 0.02 | −0.44 | 0.14 | 0.38 | −0.31 | −0.23 | −0.07 | 0.04 | 0.11 | 0.23 | 0.15 | 0.09 | 0.05 | −0.17 | −0.29 | −0.24 | 0.05 |
| 2807 | 0.05 | −0.44 | −0.15 | 0.68 | 0.33 | −0.08 | −0.24 | 0.11 | 0.19 | 0.48 | 0.12 | 0.83 | −0.53 | −0.37 | −0.12 | −0.27 | −0.24 | −0.52 |
| 2878 | 0.04 | 0.12 | −0.26 | 0.10 | −0.19 | −0.20 | 0.49 | 0.01 | −0.18 | −0.29 | −0.29 | −0.14 | −0.21 | 0.02 | −0.18 | 0.24 | −0.12 | 0.28 |
| 2879 | 0.01 | 0.47 | 0.11 | −0.01 | 0.28 | 0.39 | 0.01 | 0.13 | 0.32 | −0.07 | −0.19 | −0.10 | −0.22 | −0.04 | −0.25 | 0.58 | 0.12 | −0.24 |
| 2880 | −0.29 | −0.31 | −0.13 | 0.50 | 0.60 | −0.14 | −0.15 | 0.20 | −0.09 | 0.68 | 0.31 | 0.54 | −0.21 | −0.15 | −0.22 | −0.09 | −0.08 | −0.31 |
| 2886 | 0.20 | −0.19 | −0.13 | −0.55 | −0.42 | −0.34 | −0.28 | −0.28 | −0.22 | −0.39 | 0.02 | −0.39 | 0.56 | 0.00 | 0.37 | −0.22 | −0.16 | 0.66 |
| 2936 | 0.47 | −0.33 | −0.18 | 0.58 | −0.27 | −0.17 | −0.20 | −0.20 | −0.12 | −0.21 | −0.54 | 0.38 | −0.14 | 0.02 | −0.30 | −0.04 | −0.08 | −0.36 |
| 2953 | −0.36 | 0.07 | 0.10 | 0.50 | 0.72 | 0.66 | 0.02 | 0.79 | −0.16 | 0.76 | 0.05 | 0.54 | −0.39 | −0.29 | −0.40 | −0.16 | −0.11 | −0.24 |
| 3024 | 0.12 | −0.28 | −0.10 | −0.13 | 0.20 | −0.02 | −0.15 | −0.11 | −0.15 | −0.19 | −0.09 | −0.44 | 0.34 | 0.20 | −0.03 | −0.15 | −0.11 | 0.18 |
| 3025 | −0.07 | 0.04 | 0.19 | 0.16 | 0.45 | 0.45 | 0.10 | 0.22 | −0.13 | 0.01 | −0.18 | −0.20 | −0.01 | 0.25 | −0.32 | −0.13 | −0.09 | 0.05 |
| 3098 | 0.40 | −0.26 | −0.17 | 0.64 | −0.32 | −0.17 | −0.01 | −0.15 | −0.11 | −0.20 | −0.52 | 0.51 | −0.24 | −0.15 | −0.28 | −0.09 | −0.08 | −0.27 |
| 3099 | −0.50 | 0.01 | 0.03 | 0.55 | 0.72 | 0.41 | 0.10 | 0.58 | −0.20 | 0.68 | 0.05 | 0.54 | −0.27 | −0.11 | −0.41 | −0.14 | −0.15 | −0.00 |
| 3170 | 0.06 | 0.38 | 0.02 | −0.05 | 0.31 | −0.14 | −0.15 | −0.17 | 0.82 | −0.16 | 0.30 | 0.07 | −0.27 | −0.75 | 0.75 | −0.09 | −0.05 | −0.37 |
| 3171 | 0.04 | 0.19 | −0.13 | −0.10 | −0.17 | 0.03 | −0.08 | −0.02 | −0.09 | −0.10 | −0.38 | −0.23 | 0.01 | 0.20 | −0.22 | 0.56 | −0.05 | −0.22 |
| 3172 | 0.00 | 0.24 | 0.04 | 0.55 | 0.72 | 0.10 | 0.04 | 0.04 | 0.12 | 0.70 | 0.25 | 0.54 | −0.28 | −0.21 | −0.29 | −0.12 | −0.08 | −0.24 |
| 3390 | −0.11 | 0.08 | 0.26 | 0.18 | 0.46 | 0.57 | 0.17 | 0.30 | −0.12 | 0.06 | −0.17 | −0.12 | −0.08 | 0.12 | −0.31 | −0.12 | −0.09 | 0.10 |
| 3463 | −0.34 | 0.30 | 0.08 | 0.35 | 0.18 | 0.27 | 0.80 | 0.34 | −0.13 | −0.05 | −0.21 | 0.11 | −0.30 | −0.22 | −0.31 | −0.12 | −0.09 | 0.35 |
| | 2522 | 2528 | 2529 | 2544 | 2570 | 2571 | 2586 | 2587 | 2603 | 2644 | 2645 | 2660 | 2683 | 2714 | 2732 | 2733 | 2807 | 2878 |
| 1354 | 0.34 | −0.15 | −0.20 | −0.19 | 0.01 | 0.15 | −0.21 | −0.24 | −0.17 | 0.11 | −0.09 | −0.15 | 0.27 | −0.15 | −0.12 | −0.14 | −0.28 | −0.21 |
| 1362 | −0.01 | 0.40 | 0.29 | 0.09 | −0.05 | −0.15 | 0.15 | −0.27 | −0.01 | −0.13 | −0.08 | −0.09 | −0.06 | −0.09 | −0.07 | −0.31 | −0.30 | 0.07 |
| 1403 | 0.03 | 0.27 | 0.47 | 0.31 | −0.12 | −0.11 | 0.44 | −0.24 | 0.09 | −0.10 | −0.08 | −0.06 | −0.22 | −0.06 | −0.02 | −0.19 | −0.16 | 0.34 |
| 1475 | −0.05 | −0.27 | −0.15 | −0.24 | −0.06 | 0.15 | −0.25 | −0.23 | 0.16 | 0.36 | −0.02 | −0.20 | 0.42 | 0.58 | −0.49 | −0.08 | −0.05 | −0.02 |
| 1500 | 0.12 | −0.38 | −0.24 | −0.34 | −0.30 | 0.12 | 0.14 | −0.29 | 0.18 | 0.17 | −0.15 | −0.12 | 0.64 | 0.15 | −0.50 | −0.09 | −0.33 | −0.16 |
| 1516 | −0.16 | −0.07 | 0.26 | −0.11 | −0.05 | 0.14 | −0.09 | −0.28 | −0.14 | −0.05 | −0.15 | −0.15 | 0.12 | −0.15 | −0.11 | −0.27 | −0.39 | −0.14 |
| 1541 | 0.35 | −0.09 | −0.09 | −0.12 | −0.06 | 0.06 | −0.10 | −0.25 | −0.13 | 0.13 | −0.11 | −0.14 | 0.35 | −0.14 | −0.09 | −0.26 | −0.25 | −0.20 |
| 1549 | −0.14 | −0.03 | 0.02 | −0.01 | −0.25 | −0.22 | 0.02 | 0.29 | −0.05 | −0.31 | −0.17 | 0.97 | −0.27 | −0.08 | 0.14 | −0.05 | 0.14 | −0.04 |
| 1557 | 0.89 | −0.04 | 0.05 | 0.01 | 0.11 | −0.01 | 0.05 | −0.25 | −0.06 | 0.10 | −0.07 | −0.11 | 0.07 | −0.11 | −0.11 | 0.16 | −0.15 | 0.12 |
| 1565 | 0.16 | 0.11 | 0.25 | 0.04 | −0.44 | −0.48 | 0.35 | −0.33 | −0.35 | −0.36 | −0.27 | 0.23 | 0.28 | 0.24 | −0.30 | 0.21 | −0.62 | 0.31 |
| 1637 | −0.06 | −0.30 | −0.20 | −0.27 | −0.06 | 0.12 | −0.03 | −0.23 | 0.09 | 0.36 | −0.05 | −0.22 | 0.42 | 0.46 | −0.54 | −0.04 | −0.12 | −0.11 |
| 1678 | 0.19 | −0.28 | 0.01 | −0.29 | −0.09 | −0.13 | 0.00 | −0.43 | 0.02 | 0.10 | −0.22 | −0.15 | 0.50 | 0.46 | −0.79 | 0.11 | −0.60 | 0.03 |
| 1703 | 0.49 | −0.19 | 0.03 | −0.17 | −0.34 | 0.02 | 0.25 | −0.49 | 0.48 | 0.13 | −0.05 | −0.26 | 0.80 | 0.31 | −0.46 | −0.06 | −0.44 | 0.02 |
| 1711 | −0.28 | −0.27 | 0.13 | −0.37 | 0.07 | 0.24 | 0.14 | −0.10 | 0.14 | 0.36 | 0.35 | 0.01 | 0.20 | 0.32 | 0.07 | 0.10 | 0.30 | −0.22 |
| 1719 | 0.60 | −0.15 | 0.05 | −0.17 | −0.20 | 0.19 | 0.29 | −0.37 | 0.45 | 0.02 | −0.18 | −0.24 | 0.52 | −0.24 | −0.38 | −0.13 | −0.44 | −0.16 |
| 1727 | 0.13 | −0.03 | 0.11 | −0.04 | −0.06 | −0.36 | 0.04 | −0.35 | −0.42 | −0.12 | −0.19 | 0.16 | 0.07 | 0.48 | −0.57 | 0.36 | −0.47 | 0.29 |
| 1744 | −0.33 | −0.11 | 0.30 | −0.14 | 0.14 | −0.01 | −0.12 | −0.16 | −0.15 | 0.09 | −0.23 | −0.16 | 0.03 | −0.16 | −0.21 | −0.49 | −0.41 | −0.23 |
| 1768 | 0.07 | 0.05 | 0.53 | −0.05 | 0.21 | −0.24 | 0.00 | −0.34 | −0.11 | −0.14 | −0.13 | −0.16 | −0.03 | −0.16 | −0.14 | −0.20 | −0.64 | 0.02 |
| 1791 | 0.15 | −0.16 | −0.13 | −0.12 | −0.12 | 0.21 | −0.12 | −0.19 | −0.12 | 0.12 | −0.10 | −0.12 | 0.29 | −0.12 | −0.07 | −0.20 | −0.14 | −0.19 |
| 1799 | 0.13 | −0.14 | −0.16 | −0.16 | 0.05 | −0.23 | −0.16 | −0.24 | −0.16 | 0.36 | 0.02 | −0.15 | 0.36 | 0.61 | −0.34 | −0.05 | −0.19 | 0.08 |
| 1840 | −0.08 | −0.25 | 0.04 | −0.26 | −0.02 | −0.40 | 0.16 | −0.25 | −0.24 | 0.07 | −0.09 | −0.22 | 0.20 | 0.47 | −0.44 | 0.20 | −0.53 | 0.41 |
| 1865 | 0.59 | −0.04 | 0.13 | −0.02 | −0.19 | 0.00 | 0.26 | −0.46 | 0.07 | 0.09 | −0.20 | −0.21 | 0.37 | −0.02 | −0.36 | −0.14 | −0.43 | 0.09 |
| 1873 | 0.24 | −0.41 | 0.30 | −0.43 | 0.01 | −0.14 | −0.07 | −0.39 | 0.09 | 0.01 | −0.31 | −0.15 | 0.28 | 0.16 | −0.72 | 0.03 | −0.65 | 0.22 |
| 1889 | 0.44 | −0.16 | 0.25 | −0.15 | −0.17 | −0.24 | 0.00 | −0.39 | 0.13 | −0.21 | −0.39 | 0.00 | 0.27 | 0.18 | −0.70 | 0.09 | −0.73 | 0.37 |
| 1906 | 0.17 | −0.12 | 0.02 | −0.22 | −0.11 | −0.07 | 0.29 | −0.53 | 0.01 | 0.16 | −0.19 | −0.22 | 0.55 | 0.23 | −0.73 | −0.10 | −0.59 | −0.24 |
| 1914 | 0.07 | −0.29 | 0.45 | −0.37 | −0.28 | −0.08 | 0.30 | −0.44 | 0.15 | −0.07 | −0.36 | 0.02 | 0.40 | 0.07 | −0.72 | −0.05 | −0.57 | 0.03 |
| 1930 | −0.02 | 0.38 | 0.32 | 0.40 | 0.00 | −0.32 | −0.07 | 0.26 | −0.32 | −0.45 | −0.14 | 0.30 | −0.58 | −0.29 | 0.43 | 0.03 | 0.03 | 0.39 |
| 1946 | −0.28 | 0.52 | 0.49 | 0.61 | −0.25 | −0.41 | 0.06 | 0.19 | −0.10 | −0.56 | −0.35 | 0.34 | −0.39 | −0.16 | 0.12 | −0.22 | −0.17 | 0.37 |
| 1947 | 0.25 | −0.31 | −0.39 | −0.38 | 0.02 | 0.16 | 0.14 | −0.34 | 0.09 | 0.28 | −0.07 | −0.29 | 0.46 | 0.12 | −0.64 | 0.06 | −0.36 | −0.28 |
| 2002 | −0.11 | −0.36 | 0.06 | −0.36 | −0.10 | −0.08 | 0.17 | −0.40 | 0.13 | 0.06 | −0.14 | −0.36 | 0.35 | 0.35 | −0.70 | 0.13 | −0.60 | 0.29 |
| 2010 | 0.47 | −0.12 | 0.11 | −0.17 | 0.11 | 0.12 | −0.14 | −0.31 | −0.20 | 0.17 | −0.22 | −0.21 | 0.17 | −0.21 | −0.24 | −0.24 | −0.33 | −0.18 |
| 2011 | −0.04 | 0.09 | −0.26 | 0.16 | −0.21 | −0.08 | 0.33 | 0.10 | 0.57 | −0.12 | −0.21 | −0.17 | 0.28 | −0.17 | −0.32 | −0.25 | −0.31 | −0.07 |
| 2018 | 0.46 | −0.10 | −0.10 | −0.08 | 0.27 | 0.06 | −0.09 | −0.10 | −0.07 | −0.03 | 0.01 | −0.06 | −0.22 | −0.06 | −0.11 | 0.33 | −0.19 | 0.15 |
| 2035 | −0.14 | −0.22 | 0.26 | −0.23 | −0.15 | −0.14 | 0.25 | −0.45 | −0.24 | 0.15 | −0.07 | −0.23 | 0.46 | 0.48 | −0.45 | 0.11 | −0.46 | 0.01 |
| 2052 | 0.16 | −0.46 | −0.16 | −0.47 | −0.41 | −0.13 | 0.26 | −0.35 | 0.21 | 0.02 | −0.13 | 0.06 | 0.69 | 0.49 | −0.71 | 0.31 | −0.45 | 0.13 |
| 2068 | 0.08 | −0.23 | −0.08 | −0.25 | −0.06 | −0.11 | 0.13 | −0.40 | −0.25 | 0.26 | −0.09 | −0.24 | 0.47 | 0.29 | −0.42 | −0.10 | −0.48 | −0.12 |
| 2076 | −0.12 | 0.24 | 0.29 | 0.17 | 0.04 | −0.21 | −0.01 | 0.38 | −0.27 | −0.35 | −0.02 | 0.36 | −0.56 | −0.37 | 0.54 | 0.11 | 0.28 | 0.15 |
| 2092 | −0.30 | 0.38 | 0.12 | 0.38 | −0.40 | −0.45 | 0.09 | 0.55 | 0.04 | −0.76 | −0.44 | 0.29 | −0.34 | −0.28 | 0.03 | 0.15 | −0.01 | 0.24 |
| 2117 | −0.36 | −0.34 | 0.15 | −0.37 | 0.15 | 0.18 | −0.05 | 0.13 | 0.19 | 0.10 | 0.01 | 0.10 | −0.02 | −0.01 | −0.22 | 0.08 | 0.18 | −0.35 |
| 2133 | 0.18 | 0.11 | 0.12 | 0.07 | 0.09 | −0.13 | −0.20 | −0.49 | −0.28 | 0.20 | −0.05 | −0.30 | 0.28 | 0.57 | −0.49 | 0.01 | −0.35 | 0.08 |
| 2156 | 0.62 | −0.14 | −0.08 | −0.15 | −0.15 | 0.04 | 0.12 | −0.42 | −0.05 | 0.09 | −0.25 | −0.16 | 0.39 | −0.04 | −0.46 | −0.05 | −0.46 | −0.02 |
| 2157 | 0.12 | −0.12 | −0.31 | −0.05 | −0.07 | −0.26 | 0.02 | 0.41 | 0.05 | −0.17 | −0.13 | −0.20 | −0.01 | 0.14 | −0.08 | 0.48 | 0.07 | −0.07 |
| 2164 | −0.20 | 0.49 | 0.24 | 0.44 | −0.05 | −0.45 | 0.47 | −0.17 | −0.16 | −0.27 | −0.18 | −0.17 | 0.16 | −0.17 | −0.14 | −0.24 | −0.60 | −0.24 |
| 2221 | −0.10 | 0.18 | 0.09 | 0.20 | 0.69 | 0.53 | 0.03 | −0.41 | −0.19 | 0.60 | 0.79 | −0.22 | −0.40 | −0.08 | 0.70 | −0.25 | 0.30 | 0.17 |
| 2222 | −0.31 | 0.07 | −0.21 | 0.12 | −0.47 | −0.40 | −0.12 | 0.92 | 0.03 | −0.66 | −0.39 | 0.46 | −0.33 | −0.17 | 0.22 | 0.33 | 0.43 | 0.04 |
| 2230 | 0.53 | −0.18 | 0.04 | −0.19 | −0.12 | 0.11 | 0.29 | −0.34 | 0.32 | 0.16 | −0.25 | −0.27 | 0.50 | −0.27 | −0.44 | −0.31 | −0.46 | −0.27 |
| 2237 | −0.16 | 0.52 | 0.24 | 0.55 | −0.07 | −0.25 | 0.09 | 0.06 | −0.11 | −0.47 | −0.26 | 0.10 | −0.58 | −0.19 | −0.04 | −0.05 | −0.17 | 0.53 |
| 2238 | 0.03 | −0.45 | −0.14 | −0.41 | −0.63 | −0.18 | 0.15 | 0.56 | 0.47 | −0.33 | −0.43 | 0.08 | 0.28 | −0.03 | −0.24 | 0.33 | 0.19 | −0.02 |
| 2239 | 0.29 | −0.04 | −0.18 | −0.22 | −0.21 | 0.04 | 0.39 | −0.25 | 0.40 | 0.05 | 0.00 | −0.17 | 0.57 | 0.04 | −0.43 | 0.10 | −0.20 | −0.24 |
| 2246 | −0.15 | −0.10 | −0.10 | −0.08 | −0.22 | 0.41 | −0.09 | −0.09 | −0.07 | 0.05 | −0.07 | −0.05 | 0.11 | −0.06 | −0.04 | −0.08 | 0.08 | −0.12 |
| 2253 | −0.09 | 0.58 | 0.23 | 0.61 | −0.18 | −0.30 | 0.20 | 0.01 | −0.03 | −0.41 | −0.23 | −0.13 | −0.42 | −0.13 | 0.04 | −0.17 | −0.25 | 0.68 |
| 2254 | −0.13 | 0.93 | 0.07 | 1.00 | −0.02 | −0.22 | 0.07 | 0.11 | −0.04 | −0.31 | −0.17 | −0.08 | −0.28 | −0.08 | 0.08 | −0.32 | −0.07 | 0.00 |
| 2263 | −0.40 | −0.13 | −0.23 | −0.08 | −0.49 | −0.23 | −0.07 | 0.60 | 0.22 | −0.41 | −0.32 | 0.62 | 0.02 | 0.17 | −0.18 | 0.24 | 0.30 | −0.18 |
| 2279 | 0.02 | −0.26 | 0.00 | −0.35 | 0.70 | 0.59 | −0.09 | −0.65 | −0.10 | 0.90 | 0.74 | −0.33 | 0.13 | 0.24 | 0.17 | −0.21 | 0.14 | −0.22 |
| 2280 | −0.25 | −0.06 | 0.09 | −0.14 | 0.49 | 0.35 | −0.24 | −0.59 | −0.28 | 0.63 | 0.63 | −0.15 | 0.02 | 0.55 | 0.17 | −0.15 | 0.05 | 0.04 |
| 2295 | −0.04 | 0.27 | 0.23 | 0.22 | −0.12 | −0.47 | 0.31 | −0.04 | −0.28 | −0.47 | −0.33 | 0.28 | −0.18 | −0.31 | −0.21 | 0.02 | −0.44 | 0.12 |
| 2321 | −0.03 | 0.24 | −0.01 | 0.30 | −0.07 | 0.11 | −0.19 | 0.08 | 0.75 | −0.14 | −0.15 | −0.13 | 0.28 | −0.13 | −0.19 | −0.44 | −0.15 | −0.26 |
| 2367 | −0.28 | 0.09 | −0.10 | 0.14 | 0.30 | 0.16 | −0.18 | 0.47 | −0.17 | 0.02 | 0.39 | 0.12 | −0.62 | −0.25 | 0.84 | 0.14 | 0.68 | 0.10 |
| 2368 | −0.19 | 0.26 | −0.06 | 0.32 | −0.08 | −0.29 | −0.22 | 0.76 | −0.27 | −0.58 | −0.44 | 0.15 | −0.57 | −0.30 | 0.05 | 0.38 | 0.33 | −0.19 |
| 2383 | −0.27 | 0.58 | 0.35 | 0.58 | −0.10 | −0.35 | −0.11 | 0.23 | −0.13 | −0.47 | −0.29 | 0.45 | −0.24 | −0.14 | 0.12 | −0.31 | −0.08 | −0.20 |
| 2384 | −0.15 | 0.20 | 0.07 | 0.23 | −0.25 | −0.15 | −0.18 | 0.18 | 0.40 | −0.39 | −0.27 | −0.15 | 0.00 | −0.15 | −0.05 | −0.23 | −0.24 | 0.49 |
| 2390 | −0.32 | 0.35 | −0.15 | 0.36 | −0.28 | −0.49 | −0.15 | 0.62 | −0.17 | −0.60 | −0.33 | 0.73 | −0.43 | −0.17 | 0.26 | −0.07 | 0.11 | 0.01 |
| 2400 | −0.02 | −0.15 | −0.74 | −0.12 | 0.08 | 0.38 | −0.12 | 0.02 | 0.51 | 0.05 | 0.02 | −0.09 | 0.04 | −0.09 | −0.30 | 0.04 | 0.19 | −0.18 |
| 2408 | −0.20 | 0.09 | 0.03 | 0.11 | −0.39 | −0.22 | −0.19 | 0.80 | 0.30 | −0.51 | −0.32 | 0.45 | −0.15 | −0.16 | 0.24 | 0.11 | 0.48 | −0.29 |
| 2425 | −0.13 | −0.28 | −0.15 | −0.25 | −0.27 | −0.05 | −0.15 | 0.42 | 0.33 | −0.17 | −0.42 | −0.10 | 0.22 | 0.12 | −0.55 | 0.23 | 0.12 | −0.29 |
| 2441 | −0.31 | −0.05 | −0.24 | −0.03 | 0.10 | 0.14 | −0.15 | 0.55 | −0.02 | 0.08 | 0.36 | 0.33 | −0.33 | −0.08 | 0.71 | 0.15 | 0.83 | −0.14 |
| 2447 | 0.52 | −0.15 | 0.09 | −0.20 | −0.09 | −0.08 | 0.43 | −0.42 | −0.21 | 0.04 | −0.15 | −0.21 | 0.44 | −0.21 | −0.28 | 0.09 | −0.53 | −0.21 |
| 2448 | 0.80 | −0.05 | 0.29 | −0.08 | 0.18 | −0.11 | −0.04 | −0.33 | −0.13 | 0.04 | −0.12 | −0.15 | 0.14 | −0.15 | −0.15 | 0.05 | −0.37 | 0.02 |
| 2482 | −0.04 | −0.36 | 0.04 | −0.29 | 0.02 | 0.18 | −0.31 | −0.20 | 0.20 | 0.32 | −0.10 | −0.22 | 0.44 | 0.36 | −0.49 | −0.17 | −0.12 | −0.18 |
| 2512 | 0.01 | 0.39 | 0.45 | 0.23 | −0.10 | −0.15 | 0.34 | −0.30 | 0.05 | −0.13 | −0.09 | −0.09 | −0.15 | −0.09 | −0.05 | −0.29 | −0.27 | 0.24 |
| 2513 | −0.03 | 0.29 | 0.05 | −0.08 | 0.01 | −0.11 | −0.09 | −0.16 | −0.07 | −0.08 | −0.04 | −0.06 | 0.07 | −0.06 | −0.06 | −0.24 | −0.24 | −0.12 |
| 2521 | 0.16 | 0.06 | −0.09 | 0.08 | −0.49 | −0.21 | 0.15 | −0.29 | 0.16 | −0.15 | −0.18 | −0.31 | 0.70 | 0.35 | −0.40 | 0.05 | −0.52 | 0.28 |
| 2522 | 1.00 | −0.14 | −0.12 | −0.13 | −0.01 | 0.03 | 0.07 | −0.26 | 0.19 | 0.15 | −0.05 | −0.15 | 0.40 | 0.00 | −0.25 | 0.20 | −0.16 | 0.01 |
| 2528 | −0.14 | 1.00 | 0.09 | 0.93 | −0.02 | −0.26 | 0.04 | 0.05 | −0.06 | −0.33 | −0.18 | −0.10 | −0.25 | −0.10 | 0.05 | −0.39 | −0.16 | −0.04 |
| 2529 | −0.12 | 0.09 | 1.00 | 0.08 | −0.02 | −0.15 | 0.15 | −0.27 | −0.02 | −0.17 | −0.18 | −0.10 | 0.02 | −0.10 | −0.06 | −0.18 | −0.32 | 0.05 |
| 2544 | −0.13 | 0.93 | 0.08 | 1.00 | −0.02 | −0.22 | 0.08 | 0.11 | −0.03 | −0.31 | −0.17 | −0.08 | −0.29 | −0.08 | 0.08 | −0.32 | −0.07 | 0.01 |
| 2570 | −0.01 | −0.02 | −0.02 | −0.02 | 1.00 | 0.53 | −0.26 | −0.34 | −0.24 | 0.70 | 0.66 | −0.22 | −0.38 | −0.22 | 0.45 | −0.31 | 0.18 | −0.27 |
| 2571 | 0.03 | −0.26 | −0.15 | −0.22 | 0.53 | 1.00 | −0.23 | −0.32 | 0.31 | 0.70 | 0.64 | −0.18 | −0.04 | −0.19 | 0.37 | −0.30 | 0.47 | −0.28 |
| 2586 | 0.07 | 0.04 | 0.15 | 0.08 | −0.26 | −0.23 | 1.00 | −0.25 | −0.02 | −0.08 | −0.05 | −0.09 | 0.16 | −0.09 | −0.20 | 0.20 | −0.32 | 0.06 |
| 2587 | −0.26 | 0.05 | −0.27 | 0.11 | −0.34 | −0.32 | −0.25 | 1.00 | 0.02 | −0.59 | −0.48 | 0.35 | −0.42 | −0.28 | 0.15 | 0.24 | 0.46 | −0.11 |
| 2603 | 0.19 | −0.06 | −0.02 | −0.03 | −0.24 | 0.31 | −0.02 | 0.02 | 1.00 | −0.01 | −0.08 | −0.07 | 0.36 | −0.07 | −0.22 | −0.29 | 0.03 | −0.07 |
| 2644 | 0.15 | −0.33 | −0.17 | −0.31 | 0.70 | 0.70 | −0.08 | −0.59 | −0.01 | 1.00 | 0.84 | −0.29 | 0.17 | 0.20 | 0.34 | −0.23 | 0.28 | −0.21 |
| 2645 | −0.05 | −0.18 | −0.18 | −0.17 | 0.66 | 0.64 | −0.05 | −0.48 | −0.08 | 0.84 | 1.00 | −0.15 | −0.01 | 0.23 | 0.63 | −0.09 | 0.41 | −0.06 |
| 2660 | −0.15 | −0.10 | −0.10 | −0.08 | −0.22 | −0.19 | −0.09 | 0.35 | −0.07 | −0.29 | −0.15 | 1.00 | −0.22 | −0.06 | 0.15 | −0.01 | 0.18 | −0.12 |
| 2683 | 0.40 | −0.25 | 0.02 | −0.29 | −0.38 | −0.04 | 0.16 | −0.42 | 0.36 | 0.17 | −0.01 | −0.22 | 1.00 | 0.47 | −0.44 | 0.08 | −0.32 | −0.12 |
| 2714 | 0.00 | −0.10 | −0.10 | −0.08 | −0.22 | −0.19 | −0.09 | −0.28 | −0.07 | 0.20 | 0.23 | −0.06 | 0.47 | 1.00 | −0.21 | 0.35 | −0.02 | 0.35 |
| 2732 | −0.25 | 0.05 | −0.06 | 0.08 | 0.45 | 0.37 | −0.20 | 0.15 | −0.22 | 0.34 | 0.63 | 0.15 | −0.44 | −0.21 | 1.00 | −0.17 | 0.64 | −0.09 |
| 2733 | 0.20 | −0.39 | −0.18 | −0.32 | −0.31 | −0.30 | 0.20 | 0.24 | −0.29 | −0.23 | −0.09 | −0.01 | 0.08 | 0.35 | −0.17 | 1.00 | 0.17 | 0.27 |
| 2807 | −0.16 | −0.16 | −0.32 | −0.07 | 0.18 | 0.47 | −0.32 | 0.46 | 0.03 | 0.28 | 0.41 | 0.18 | −0.32 | −0.02 | 0.64 | 0.17 | 1.00 | −0.22 |
| 2878 | 0.01 | −0.04 | 0.06 | 0.01 | −0.27 | −0.28 | 0.06 | −0.11 | −0.07 | −0.21 | −0.06 | −0.12 | −0.12 | 0.35 | −0.09 | 0.27 | −0.22 | 1.00 |
| 2879 | −0.16 | 0.72 | 0.21 | 0.71 | 0.07 | −0.09 | 0.17 | −0.08 | −0.04 | −0.23 | −0.12 | −0.13 | −0.38 | −0.13 | −0.09 | −0.22 | −0.07 | 0.05 |
| 2880 | −0.15 | −0.10 | −0.10 | −0.08 | −0.22 | −0.19 | −0.09 | 0.12 | −0.07 | −0.29 | −0.15 | −0.06 | −0.22 | −0.06 | 0.31 | 0.46 | 0.56 | −0.12 |
| 2886 | 0.15 | −0.26 | −0.24 | −0.21 | −0.32 | −0.04 | 0.23 | −0.37 | −0.18 | 0.21 | 0.07 | −0.16 | 0.68 | 0.50 | −0.29 | 0.23 | −0.23 | −0.02 |
| 2936 | −0.01 | −0.11 | −0.08 | −0.08 | 0.75 | 0.65 | −0.07 | −0.33 | −0.08 | 0.72 | 0.90 | −0.08 | −0.29 | −0.08 | 0.17 | −0.17 | 0.43 | −0.05 |
| 2953 | −0.27 | 0.50 | −0.18 | 0.56 | −0.24 | −0.36 | −0.16 | 0.77 | −0.13 | −0.52 | −0.27 | 0.45 | −0.40 | −0.11 | 0.33 | 0.09 | 0.42 | −0.23 |
| 3024 | 0.16 | 0.07 | −0.18 | 0.11 | 0.02 | 0.31 | −0.15 | −0.08 | −0.12 | −0.05 | −0.09 | −0.11 | −0.12 | −0.11 | −0.08 | 0.10 | −0.07 | −0.02 |
| 3025 | 0.20 | 0.50 | −0.14 | 0.55 | 0.20 | −0.11 | −0.13 | 0.09 | −0.11 | −0.24 | −0.11 | −0.09 | −0.32 | −0.09 | −0.01 | 0.03 | −0.14 | 0.01 |
| 3098 | −0.15 | −0.12 | −0.11 | −0.09 | 0.55 | 0.52 | −0.10 | −0.25 | −0.09 | 0.59 | 0.89 | −0.08 | −0.27 | −0.08 | 0.83 | −0.22 | 0.48 | 0.04 |
| 3099 | 0.45 | −0.18 | 0.50 | −0.27 | −0.38 | −0.10 | 0.79 | −0.15 | −0.49 | −0.50 | −0.14 | −0.32 | −0.14 | 0.34 | 0.22 | 0.47 | −0.09 | 0.17 |
| 3170 | −0.75 | −0.70 | −0.70 | −0.08 | 0.24 | 0.22 | −0.09 | −0.02 | −0.07 | 0.05 | 0.07 | −0.05 | −0.22 | −0.05 | −0.27 | 0.23 | 0.78 | −0.72 |
| 3171 | 0.03 | 0.27 | 0.47 | 0.37 | −0.72 | −0.77 | 0.44 | −0.24 | 0.09 | −0.70 | −0.08 | −0.05 | −0.22 | −0.05 | −0.02 | −0.79 | −0.75 | 0.34 |
| 3172 | −0.19 | 0.23 | −0.13 | 0.27 | −0.20 | −0.25 | −0.12 | 0.77 | −0.10 | −0.38 | −0.20 | −0.08 | −0.29 | −0.08 | 0.33 | 0.34 | 0.53 | −0.15 |
| 3390 | 0.09 | 0.74 | −0.13 | 0.80 | 0.15 | −0.15 | −0.12 | 0.14 | −0.10 | −0.27 | −0.13 | −0.09 | −0.30 | −0.09 | 0.03 | −0.08 | −0.10 | −0.04 |
| 3453 | −0.21 | 0.39 | −0.14 | 0.44 | −0.18 | −0.28 | −0.12 | 0.27 | −0.10 | −0.40 | −0.21 | −0.09 | −0.31 | −0.09 | 0.10 | −0.03 | −0.10 | 0.51 |

| | 2879 | 2880 | 2886 | 2936 | 2953 | 3024 | 3025 | 3098 | 3099 | 3170 | 3171 | 3172 | 3390 | 3463 |
|---|---|---|---|---|---|---|---|---|---|---|---|---|---|---|
| 1354 | −0.25 | −0.15 | 0.57 | −0.10 | −0.27 | 0.54 | 0.01 | −0.17 | −0.17 | −0.15 | −0.15 | −0.19 | −0.04 | −0.21 |
| 1362 | 0.42 | −0.09 | −0.22 | −0.07 | −0.16 | −0.15 | −0.13 | −0.10 | −0.16 | −0.09 | 0.47 | −0.12 | −0.12 | −0.13 |
| 1403 | 0.58 | −0.06 | −0.16 | 0.00 | −0.11 | −0.11 | −0.09 | −0.05 | −0.08 | −0.06 | 1.00 | −0.08 | −0.09 | −0.09 |
| 1475 | −0.19 | −0.20 | 0.44 | −0.27 | −0.37 | 0.01 | −0.29 | −0.25 | −0.38 | 0.09 | −0.14 | −0.26 | −0.28 | −0.28 |
| 1500 | −0.32 | −0.30 | 0.80 | −0.37 | −0.45 | 0.17 | −0.39 | −0.34 | −0.44 | −0.01 | −0.14 | −0.39 | −0.38 | −0.27 |
| 1516 | −0.10 | −0.15 | 0.42 | −0.12 | −0.28 | 0.55 | −0.06 | −0.18 | −0.32 | −0.15 | 0.09 | −0.20 | −0.10 | −0.22 |
| 1541 | −0.12 | −0.14 | 0.56 | −0.17 | −0.26 | 0.22 | −0.20 | −0.17 | −0.10 | −0.14 | 0.05 | −0.19 | −0.19 | −0.20 |
| 1549 | 0.02 | −0.08 | −0.19 | −0.08 | 0.42 | −0.13 | −0.11 | −0.09 | 0.12 | −0.08 | 0.19 | −0.10 | −0.11 | −0.11 |
| 1557 | 0.04 | −0.11 | 0.03 | 0.07 | −0.20 | 0.24 | 0.27 | −0.10 | 0.01 | −0.11 | 0.30 | −0.15 | 0.15 | −0.16 |
| 1565 | −0.08 | −0.36 | 0.43 | −0.28 | −0.09 | 0.30 | 0.15 | −0.33 | −0.19 | −0.36 | 0.11 | −0.35 | 0.10 | 0.10 |
| 1637 | −0.21 | −0.22 | 0.51 | −0.29 | −0.40 | 0.01 | −0.32 | −0.28 | −0.42 | 0.11 | −0.16 | −0.29 | −0.30 | −0.31 |
| 1678 | −0.23 | −0.47 | 0.49 | −0.40 | −0.52 | 0.23 | 0.01 | −0.50 | −0.61 | 0.08 | −0.20 | −0.53 | −0.07 | −0.28 |
| 1703 | −0.18 | −0.26 | 0.63 | −0.23 | −0.47 | 0.09 | −0.18 | −0.30 | −0.40 | −0.26 | 0.21 | −0.34 | −0.22 | −0.36 |
| 1711 | −0.26 | 0.12 | −0.06 | 0.22 | −0.15 | −0.29 | −0.39 | 0.23 | −0.27 | −0.01 | −0.16 | 0.00 | −0.39 | −0.42 |
| 1719 | −0.16 | −0.24 | 0.37 | −0.14 | −0.44 | 0.38 | 0.01 | −0.27 | −0.36 | −0.24 | 0.17 | −0.32 | −0.08 | −0.34 |
| 1727 | 0.16 | −0.43 | 0.26 | −0.31 | −0.21 | 0.21 | 0.22 | −0.42 | −0.35 | 0.29 | 0.05 | −0.43 | 0.13 | −0.08 |
| 1744 | −0.14 | −0.16 | 0.21 | −0.20 | −0.30 | 0.12 | −0.24 | −0.20 | −0.35 | −0.16 | 0.06 | −0.22 | −0.23 | −0.23 |
| 1768 | 0.06 | −0.16 | −0.09 | −0.01 | −0.29 | 0.12 | 0.16 | −0.16 | −0.32 | −0.16 | 0.28 | −0.21 | 0.05 | −0.22 |
| 1791 | −0.18 | −0.12 | 0.66 | −0.15 | −0.22 | 0.45 | −0.17 | −0.15 | −0.12 | −0.12 | −0.02 | −0.16 | −0.16 | −0.17 |
| 1799 | −0.20 | −0.15 | 0.40 | −0.19 | −0.27 | −0.25 | −0.22 | −0.18 | −0.21 | −0.15 | −0.06 | −0.20 | −0.21 | −0.21 |
| 1840 | −0.34 | −0.22 | 0.25 | −0.18 | −0.41 | −0.12 | −0.06 | −0.17 | −0.40 | −0.22 | −0.12 | −0.29 | −0.12 | 0.10 |
| 1865 | 0.09 | −0.29 | 0.47 | −0.21 | −0.44 | 0.26 | −0.10 | −0.31 | −0.28 | −0.18 | 0.51 | −0.36 | −0.15 | −0.27 |
| 1873 | −0.18 | −0.45 | 0.10 | −0.35 | −0.67 | 0.02 | −0.14 | −0.42 | −0.66 | 0.15 | 0.02 | −0.60 | −0.27 | −0.18 |
| 1889 | −0.06 | −0.54 | 0.13 | −0.36 | −0.47 | 0.28 | 0.21 | −0.49 | −0.52 | −0.02 | 0.13 | −0.60 | 0.07 | −0.02 |
| 1906 | 0.03 | −0.41 | 0.58 | −0.42 | −0.55 | 0.07 | −0.25 | −0.50 | −0.59 | 0.15 | 0.07 | −0.49 | −0.27 | −0.50 |
| 1914 | 0.02 | −0.48 | 0.22 | −0.48 | −0.61 | −0.05 | −0.43 | −0.49 | −0.69 | 0.20 | 0.23 | −0.63 | −0.48 | −0.34 |
| 1930 | 0.24 | 0.09 | −0.54 | 0.15 | 0.44 | 0.04 | 0.41 | 0.16 | 0.44 | −0.24 | 0.26 | 0.20 | 0.40 | 0.53 |
| 1946 | 0.44 | −0.16 | −0.40 | −0.17 | 0.42 | −0.11 | 0.26 | −0.08 | 0.31 | −0.16 | 0.41 | 0.03 | 0.36 | 0.58 |
| 1947 | −0.20 | −0.29 | 0.54 | −0.27 | −0.53 | 0.24 | −0.13 | −0.35 | −0.53 | 0.23 | −0.29 | −0.38 | −0.20 | −0.41 |
| 2002 | −0.16 | −0.36 | 0.35 | −0.31 | −0.66 | 0.07 | −0.18 | −0.33 | −0.69 | 0.18 | −0.05 | −0.48 | −0.26 | −0.07 |
| 2010 | −0.16 | −0.21 | 0.33 | −0.14 | −0.39 | 0.45 | −0.01 | −0.24 | −0.25 | −0.21 | 0.09 | −0.28 | −0.09 | −0.30 |
| 2011 | −0.11 | −0.17 | 0.04 | −0.23 | −0.03 | −0.19 | 0.06 | −0.15 | −0.06 | −0.17 | −0.17 | −0.07 | 0.13 | 0.26 |
| 2018 | −0.13 | −0.06 | −0.16 | 0.23 | −0.11 | 0.62 | 0.64 | −0.03 | −0.14 | −0.06 | −0.06 | −0.08 | 0.43 | −0.09 |
| 2035 | −0.29 | −0.23 | 0.62 | −0.22 | −0.42 | 0.23 | −0.16 | −0.27 | −0.50 | −0.23 | −0.04 | −0.30 | −0.19 | −0.33 |
| 2052 | −0.33 | −0.40 | 0.59 | −0.41 | −0.49 | −0.04 | −0.31 | −0.40 | −0.58 | 0.14 | −0.24 | −0.53 | −0.37 | −0.20 |
| 2068 | −0.31 | −0.24 | 0.76 | −0.26 | −0.44 | 0.19 | −0.23 | −0.29 | −0.42 | −0.24 | −0.06 | −0.32 | −0.25 | −0.34 |
| 2076 | 0.14 | 0.28 | −0.67 | 0.23 | 0.46 | −0.20 | 0.14 | 0.27 | 0.46 | −0.11 | 0.10 | 0.32 | 0.15 | 0.34 |
| 2092 | 0.26 | 0.18 | −0.50 | −0.31 | 0.55 | −0.18 | 0.25 | −0.19 | 0.49 | 0.05 | −0.06 | 0.32 | 0.33 | 0.66 |
| 2117 | −0.11 | 0.04 | −0.31 | −0.07 | −0.11 | −0.30 | −0.30 | −0.06 | −0.28 | 0.47 | −0.32 | −0.06 | −0.31 | −0.32 |
| 2133 | 0.26 | −0.30 | 0.41 | −0.26 | −0.33 | 0.15 | 0.07 | −0.35 | −0.30 | 0.21 | 0.15 | −0.28 | 0.05 | −0.26 |
| 2156 | −0.10 | −0.36 | 0.58 | −0.26 | −0.45 | 0.48 | 0.01 | −0.37 | −0.34 | −0.13 | 0.16 | −0.42 | −0.07 | −0.26 |
| 2157 | −0.28 | 0.61 | 0.05 | −0.07 | 0.27 | 0.18 | 0.35 | −0.22 | 0.35 | −0.20 | −0.20 | 0.59 | 0.25 | −0.16 |
| 2164 | 0.25 | −0.17 | 0.16 | −0.21 | 0.12 | −0.13 | 0.23 | −0.21 | 0.04 | −0.17 | 0.04 | 0.01 | 0.32 | 0.10 |
| 2221 | 0.20 | −0.21 | −0.28 | 0.89 | −0.15 | 0.04 | 0.17 | 0.89 | −0.14 | −0.04 | 0.22 | −0.15 | 0.17 | 0.15 |
| 2222 | −0.08 | 0.65 | −0.34 | −0.29 | 0.79 | −0.19 | 0.05 | −0.18 | 0.77 | −0.05 | −0.22 | 0.70 | 0.11 | 0.38 |
| 2230 | −0.20 | −0.27 | 0.39 | −0.24 | −0.49 | 0.15 | −0.19 | −0.31 | −0.37 | −0.27 | 0.17 | −0.35 | −0.23 | −0.38 |
| 2237 | 0.76 | −0.19 | −0.49 | −0.12 | 0.20 | 0.00 | 0.34 | −0.09 | 0.16 | 0.31 | 0.54 | −0.05 | 0.37 | 0.57 |
| 2238 | −0.46 | 0.56 | 0.07 | −0.44 | 0.11 | −0.21 | −0.43 | −0.37 | 0.23 | −0.19 | −0.12 | 0.39 | −0.44 | −0.11 |
| 2239 | 0.03 | −0.17 | 0.15 | −0.23 | −0.31 | −0.28 | −0.24 | −0.21 | −0.29 | 0.24 | −0.17 | −0.22 | −0.23 | −0.24 |
| 2246 | −0.13 | −0.06 | 0.51 | −0.08 | −0.11 | 0.71 | −0.09 | −0.08 | −0.14 | −0.06 | −0.06 | −0.08 | −0.09 | −0.09 |
| 2253 | 0.59 | −0.13 | −0.33 | −0.08 | 0.13 | 0.00 | 0.30 | −0.01 | 0.21 | −0.13 | 0.64 | 0.02 | 0.36 | 0.69 |
| 2254 | 0.70 | −0.08 | −0.20 | −0.08 | 0.57 | 0.12 | 0.66 | −0.09 | 0.50 | −0.08 | 0.29 | 0.27 | 0.81 | 0.44 |
| 2263 | −0.10 | 0.25 | −0.07 | −0.43 | 0.51 | −0.26 | −0.19 | −0.35 | 0.27 | 0.21 | −0.32 | 0.25 | −0.12 | −0.02 |
| 2279 | −0.06 | −0.29 | 0.12 | 0.58 | −0.60 | −0.12 | −0.29 | 0.53 | −0.60 | 0.24 | −0.04 | −0.41 | −0.34 | −0.50 |
| 2280 | 0.01 | −0.29 | 0.20 | 0.47 | −0.36 | 0.12 | −0.07 | 0.40 | −0.50 | 0.06 | 0.04 | −0.33 | −0.11 | −0.34 |
| 2295 | 0.47 | −0.31 | −0.19 | −0.33 | 0.07 | −0.28 | 0.04 | −0.26 | 0.01 | 0.38 | 0.19 | −0.24 | 0.08 | 0.30 |
| 2321 | 0.11 | −0.13 | −0.13 | −0.18 | 0.10 | −0.10 | 0.19 | −0.17 | 0.03 | 0.02 | −0.13 | 0.01 | 0.26 | 0.08 |
| 2367 | −0.01 | 0.50 | −0.55 | 0.58 | 0.50 | −0.13 | 0.16 | 0.54 | 0.55 | −0.05 | −0.10 | 0.55 | 0.18 | 0.35 |
| 2368 | 0.28 | 0.50 | −0.42 | −0.27 | 0.72 | 0.20 | 0.45 | −0.32 | 0.72 | 0.31 | −0.17 | 0.72 | 0.46 | 0.78 |
| 2383 | 0.39 | −0.14 | −0.34 | −0.71 | 0.55 | −0.02 | 0.45 | −0.17 | 0.41 | −0.14 | 0.03 | 0.13 | 0.57 | 0.27 |
| 2384 | 0.01 | −0.15 | −0.28 | −0.20 | 0.02 | −0.15 | 0.10 | −0.01 | 0.10 | −0.15 | −0.08 | −0.04 | 0.17 | 0.80 |
| 2390 | 0.13 | 0.20 | −0.28 | −0.20 | 0.79 | −0.11 | 0.22 | −0.15 | 0.58 | −0.17 | −0.02 | 0.34 | 0.30 | 0.34 |
| 2400 | 0.32 | −0.09 | −0.22 | −0.12 | −0.16 | −0.15 | −0.13 | −0.11 | −0.20 | 0.82 | −0.09 | −0.12 | −0.12 | −0.13 |
| 2408 | −0.07 | 0.68 | −0.39 | −0.21 | 0.76 | −0.19 | 0.01 | −0.20 | 0.68 | −0.16 | −0.10 | 0.70 | 0.06 | −0.05 |
| 2425 | −0.19 | 0.31 | 0.02 | −0.54 | 0.05 | −0.09 | −0.18 | −0.52 | 0.05 | 0.30 | −0.38 | 0.25 | −0.17 | −0.21 |
| 2441 | −0.10 | 0.54 | −0.39 | 0.38 | 0.54 | −0.44 | −0.20 | 0.51 | 0.54 | 0.07 | −0.23 | 0.54 | −0.12 | 0.11 |
| 2447 | −0.22 | −0.21 | 0.55 | −0.14 | −0.39 | 0.34 | −0.01 | −0.24 | −0.27 | −0.21 | 0.01 | −0.28 | −0.08 | −0.30 |
| 2448 | −0.04 | −0.16 | 0.00 | 0.02 | −0.29 | 0.20 | 0.25 | −0.16 | −0.11 | −0.16 | 0.20 | −0.21 | 0.12 | −0.22 |
| 2482 | −0.25 | −0.22 | 0.37 | −0.30 | −0.40 | −0.03 | −0.32 | −0.28 | −0.41 | 0.15 | −0.22 | −0.29 | −0.31 | −0.31 |
| 2512 | 0.58 | −0.09 | −0.22 | −0.04 | −0.16 | −0.15 | −0.13 | −0.09 | −0.14 | −0.09 | 0.86 | −0.12 | −0.12 | −0.12 |
| 2513 | 0.12 | −0.06 | −0.16 | −0.08 | −0.11 | −0.1 | −0.09 | −0.08 | −0.14 | −0.06 | −0.06 | −0.08 | −0.09 | −0.09 |
| 2521 | −0.24 | −0.31 | 0.66 | −0.36 | −0.24 | 0.18 | 0.05 | −0.27 | −0.15 | −0.31 | −0.22 | −0.24 | 0.10 | 0.35 |
| 2522 | −0.16 | −0.15 | 0.15 | −0.01 | −0.27 | 0.16 | 0.20 | −0.15 | −0.06 | −0.15 | 0.03 | −0.19 | 0.09 | −0.21 |
| 2528 | 0.72 | −0.10 | −0.25 | −0.11 | 0.50 | 0.07 | 0.50 | −0.12 | 0.43 | −0.10 | 0.27 | 0.23 | 0.74 | 0.39 |
| 2529 | 0.21 | −0.10 | −0.24 | −0.08 | −0.16 | −0.16 | −0.14 | −0.11 | −0.16 | −0.10 | 0.47 | −0.13 | −0.13 | −0.14 |
| 2544 | 0.71 | −0.08 | −0.21 | −0.08 | 0.56 | 0.11 | 0.66 | −0.09 | 0.50 | −0.08 | 0.31 | 0.27 | 0.80 | 0.44 |
| 2570 | 0.07 | −0.22 | −0.32 | 0.75 | −0.24 | 0.02 | 0.20 | 0.66 | −0.27 | 0.24 | −0.12 | −0.20 | 0.15 | −0.18 |
| 2571 | −0.09 | −0.19 | −0.04 | 0.65 | −0.35 | 0.31 | −0.11 | 0.62 | −0.38 | 0.22 | −0.11 | −0.26 | −0.15 | −0.28 |
| 2586 | 0.17 | −0.09 | 0.23 | −0.07 | −0.16 | −0.15 | −0.13 | −0.10 | −0.16 | −0.09 | 0.44 | −0.12 | −0.12 | −0.12 |
| 2587 | −0.08 | 0.72 | −0.37 | −0.33 | 0.77 | −0.08 | 0.09 | −0.26 | 0.79 | −0.02 | −0.24 | 0.77 | 0.14 | 0.27 |
| 2603 | −0.04 | −0.07 | −0.18 | −0.08 | −0.13 | −0.12 | −0.11 | −0.09 | −0.15 | −0.07 | 0.09 | −0.10 | −0.10 | −0.10 |
| 2644 | −0.23 | −0.29 | 0.21 | 0.72 | −0.52 | −0.05 | −0.24 | 0.69 | −0.49 | 0.05 | −0.10 | −0.38 | −0.27 | −0.40 |
| 2645 | −0.12 | −0.15 | 0.07 | 0.90 | −0.27 | −0.09 | −0.11 | 0.89 | −0.30 | 0.07 | −0.08 | −0.20 | −0.13 | −0.21 |
| 2660 | −0.13 | −0.06 | −0.16 | −0.08 | 0.46 | −0.11 | −0.09 | −0.08 | 0.14 | −0.06 | −0.06 | −0.08 | −0.09 | −0.09 |
| 2683 | −0.38 | −0.22 | 0.68 | −0.29 | −0.40 | −0.12 | −0.32 | −0.27 | −0.32 | −0.22 | −0.22 | −0.29 | −0.30 | −0.31 |
| 2714 | −0.13 | −0.06 | 0.50 | −0.08 | −0.11 | −0.11 | −0.09 | −0.08 | −0.14 | −0.06 | −0.06 | −0.08 | −0.09 | −0.09 |
| 2732 | −0.09 | 0.31 | −0.29 | 0.77 | 0.33 | −0.08 | −0.01 | 0.83 | 0.34 | −0.21 | −0.02 | 0.33 | 0.03 | 0.10 |
| 2733 | −0.22 | 0.46 | 0.23 | −0.17 | 0.09 | 0.10 | 0.03 | −0.22 | 0.22 | 0.23 | −0.19 | 0.34 | −0.08 | −0.03 |
| 2807 | −0.07 | 0.56 | −0.23 | 0.43 | 0.42 | −0.07 | −0.14 | 0.48 | 0.47 | 0.18 | −0.16 | 0.53 | −0.10 | −0.10 |
| 2878 | 0.05 | −0.12 | −0.02 | −0.05 | −0.23 | −0.02 | 0.01 | 0.04 | −0.09 | −0.12 | 0.34 | −0.16 | −0.04 | 0.61 |
| 2879 | 1.00 | −0.13 | −0.33 | −0.12 | 0.22 | −0.06 | 0.31 | −0.15 | 0.17 | 0.47 | 0.58 | 0.07 | 0.41 | 0.17 |
| 2880 | −0.13 | 1.00 | −0.16 | −0.08 | 0.52 | −0.11 | −0.09 | −0.08 | 0.70 | −0.06 | −0.06 | 0.93 | −0.09 | −0.09 |
| 2886 | −0.33 | −0.16 | 1.00 | −0.21 | −0.29 | 0.24 | −0.23 | −0.20 | −0.24 | −0.16 | −0.16 | −0.21 | −0.22 | −0.22 |
| 2936 | −0.12 | −0.08 | −0.21 | 1.00 | −0.16 | 0.07 | 0.09 | 0.94 | −0.18 | −0.08 | 0.00 | −0.11 | 0.03 | −0.12 |
| 2953 | 0.22 | 0.52 | −0.29 | −0.15 | 1.00 | −0.01 | 0.41 | −0.15 | 0.88 | −0.11 | −0.11 | 0.73 | 0.52 | 0.24 |
| 3024 | −0.06 | −0.11 | 0.24 | 0.16 | −0.01 | 1.09 | 0.55 | −0.10 | −0.06 | −0.11 | −0.11 | −0.04 | 0.44 | −0.01 |
| 3025 | 0.31 | −0.09 | −0.23 | 0.09 | 0.41 | 0.55 | 1.00 | −0.08 | 0.33 | −0.09 | 0.09 | 0.18 | 0.97 | 0.31 |
| 3098 | −0.15 | −0.08 | 0.83 | −0.22 | 0.15 | 0.10 | −0.15 | −0.08 | −0.20 | −0.08 | −0.05 | −0.10 | −0.08 | 0.07 |
| 3099 | 0.70 | −0.14 | −0.18 | 0.88 | −0.00 | 0.33 | −0.14 | 1.00 | −0.14 | −0.14 | −0.08 | 0.88 | 0.43 | 0.34 |
| 3170 | 0.47 | −0.05 | −0.75 | −0.08 | −0.11 | −0.77 | −0.09 | −0.08 | −0.74 | 1.00 | −0.06 | −0.08 | −0.09 | −0.09 |
| 3171 | 0.58 | −0.05 | −0.75 | 0.00 | −0.11 | −0.77 | −0.09 | −0.05 | −0.08 | −0.06 | 1.00 | −0.08 | −0.09 | −0.09 |
| 3172 | 0.07 | 0.93 | −0.21 | −0.11 | −0.73 | −0.04 | 0.18 | −0.10 | 0.88 | −0.08 | −0.08 | 1.00 | 0.24 | 0.10 |
| 3390 | 0.41 | −0.09 | −0.22 | 0.03 | 0.52 | 0.44 | 0.97 | −0.09 | 0.43 | −0.09 | −0.09 | 0.24 | 1.00 | 0.40 |
| 3453 | 0.17 | −0.09 | −0.22 | −0.12 | 0.24 | −0.01 | 0.31 | 0.07 | 0.34 | −0.09 | −0.09 | 0.10 | 0.40 | 1.00 |

TABLE 32
Discriminant Function Analysis Summary, Step 10, N of vars in
model: 10; Grouping: Dfdegr (3 grps) Wilks' Lambda:
.00021 approx. F (20, 10) = 34.077 p < .0000

| | Wilks&apos; | Partial | F-remove | p-level | Toler. | 1-Toler. |
|---|---|---|---|---|---|---|
| 2028 | 0.000356 | 0.257847 | 7.1957 | 0.033760 | 0.076143 | 0.923857 |
| 1393 | 0.003356 | 0.027331 | 88.9709 | 0.000123 | 0.010301 | 0.989699 |
| 1825 | 0.000415 | 0.221021 | 8.8112 | 0.022966 | 0.074734 | 0.925266 |
| 1419 | 0.000623 | 0.147184 | 14.4856 | 0.008311 | 0.115077 | 0.884923 |
| 1688 | 0.000816 | 0.112369 | 19.7481 | 0.004233 | 0.036621 | 0.963379 |
| 1540 | 0.004796 | 0.019125 | 128.2203 | 0.000051 | 0.005699 | 0.994301 |
| 1905 | 0.000941 | 0.097453 | 23.1533 | 0.002965 | 0.015289 | 0.984711 |
| 892 | 0.001987 | 0.046168 | 51.6506 | 0.000458 | 0.006722 | 0.993278 |
| 1095 | 0.001122 | 0.081712 | 28.0953 | 0.001909 | 0.023183 | 0.976816 |
| 1054 | 0.000443 | 0.206883 | 9.5841 | 0.019467 | 0.033402 | 0.966598 |

TABLE 33
p-Levels for Pairwise Comparison of Dependent Variable

| | hESC | EB | St3 |
|---|---|---|---|
| hESC | | 0.000030 | 0.001469 |
| EB | 0.000030 | | 0.000004 |
| St3 | 0.001469 | 0.000004 | |

TABLE 34
Chi-Square Tests with Successive Roots Removed

| | Eigen- | Canonicl | Wilks&apos; | Chi-Sqr. | df | p-level |
|---|---|---|---|---|---|---|
| 0 | 543.6531 | 0.999082 | 0.000092 | 88.31998 | 20 | 0.000000 |
| 1 | 19.0192 | 0.974704 | 0.049952 | 28.46856 | 9 | 0.000796 |

TABLE 35
Raw Coefficients for Canonical Variables

| | Root 1 | Root 2 |
|---|---|---|
| 2028 | 7.5848 | 31.1129 |
| 1393 | −87.7220 | −17.5404 |
| 1825 | −20.3737 | 1.2276 |
| 1419 | −1.6112 | 0.6578 |
| 1688 | 26.9100 | −22.0977 |
| 1540 | −23.8102 | 2.0084 |
| 1905 | 2.4675 | −1.3916 |
| 892 | 22.1050 | 6.1419 |
| 1095 | −19.1659 | −10.0060 |
| 1054 | −3.6582 | −3.6138 |
| Constant | 35.8460 | 32.4125 |
| Eigenval | 543.6531 | 19.0192 |
| Cum. Prop | 0.9662 | 1.0000 |

TABLE 36
Means of Canonical Variables

| | Root 1 | Root 2 |
|---|---|---|
| hESC | 9.6048 | 6.90485 |
| EB | −24.6352 | −1.06955 |
| St3 | 22.3379 | −3.35542 |

TABLE 37
Five discriminative masses for embryonic stem cells,
Eigenvalues, canonical means and raw coefficients.

| | Wilks&apos; | Partial | F-remove | p-level | Toler. | 1-Toler. |
|---|---|---|---|---|---|---|
| 892 | 0.037703 | 0.371871 | 8.44552 | 0.007112 | 0.252464 | 0.747536 |
| 1540 | 0.076441 | 0.183420 | 22.25982 | 0.000208 | 0.112403 | 0.887597 |
| 1905 | 0.116818 | 0.120023 | 36.65870 | 0.000025 | 0.114965 | 0.885035 |
| 1393 | 0.052729 | 0.265901 | 13.80400 | 0.001329 | 0.236783 | 0.763217 |
| 1688 | 0.037126 | 0.377655 | 8.23959 | 0.007682 | 0.202564 | 0.797436 |

| | Eigen- | Canonicl | Wilks&apos; | Chi-Sqr. | df | p-level |
|---|---|---|---|---|---|---|
| 0 | 24.17569 | 0.979938 | 0.014021 | 51.20657 | 10 | 0.000000 |
| 1 | 1.83300 | 0.804374 | 0.352983 | 12.49602 | 4 | 0.014020 |

| | Root 1 | Root 2 |
|---|---|---|
| hESC | −1.52086 | −2.17499 |
| EB | 5.10674 | 0.42329 |
| St3 | −4.94395 | 0.95615 |

| | Root 1 | Root 2 |
|---|---|---|
| 892 | −2.9664 | 1.06236 |
| 1540 | 4.9385 | 0.98374 |
| 1905 | −1.0331 | −0.05027 |
| 1393 | 16.5002 | −0.73876 |
| 1688 | −11.7267 | 5.32870 |
| Constant | 15.5575 | −2.30082 |
| Eigenval | 24.1757 | 1.83300 |
| Cum. Prop | 0.9295 | 1.00000 |

TABLE 38
Four discriminative masses for embryonic stem cells, Eigenvalues,
canonical means, their p values and raw coefficients.

| | Wilks&apos; | Partial | F-remove | p-level | Toler. | 1-Toler. |
|---|---|---|---|---|---|---|
| 892 | 0.154395 | 0.341522 | 10.60439 | 0.002715 | 0.330624 | 0.669376 |
| 1540 | 0.158162 | 0.333388 | 10.99728 | 0.002378 | 0.220196 | 0.779804 |
| 1905 | 0.186838 | 0.282219 | 13.98840 | 0.000951 | 0.280169 | 0.719831 |
| 1688 | 0.070024 | 0.753016 | 1.80397 | 0.210098 | 0.445690 | 0.554310 |

| | Eigen- | Canonicl | Wilks&apos; | Chi-Sqr. | df | p-level |
|---|---|---|---|---|---|---|
| 0 | 5.732435 | 0.922749 | 0.052729 | 36.78228 | 8 | 0.000013 |
| 1 | 1.816924 | 0.803121 | 0.354997 | 12.94557 | 3 | 0.004756 |

| Means | Root 1 | Root 2 |
|---|---|---|
| hESC | 0.96322 | −2.13749 |
| EB | −2.52606 | 0.33923 |
| St3 | 2.30492 | 1.02923 |

| P values | hESC | EB | St3 |
|---|---|---|---|
| hESC | | 0.001653 | 0.010579 |
| EB | 0.001653 | | 0.000192 |
| St3 | 0.010579 | 0.000192 | |

| | Root 1 | Root 2 |
|---|---|---|
| 892 | 2.7193 | 1.27734 |
| 1540 | −3.3607 | 0.79112 |
| 1905 | 0.6350 | −0.01744 |
| 1688 | 3.3118 | 5.25235 |
| Constant | −10.6166 | −2.94827 |
| Eigenval | 5.7324 | 1.81692 |
| Cum. Prop | 0.7593 | 1.00000 |

TABLE 39
Factors identified for combined neutral and acidic glycans.

| | 24.40 | 11.75 | 10.76 | 8.22 | 7.00 | 6.06 | 5.41 | 5.27 |
|---|---|---|---|---|---|---|---|---|
| | Fa1 | Fa2 | Fa3 | Fa4 | Fa5 | Fa6 | Fa7 | Fa8 |
| 609 | −0.57 | 0.11 | 0.07 | 0.32 | 0.12 | −0.02 | 0.07 | 0.13 |
| 730 | 0.12 | −0.15 | −0.30 | −0.69 | 0.00 | −0.20 | 0.17 | 0.01 |
| 771 | 0.56 | −0.01 | −0.08 | −0.52 | −0.31 | 0.03 | 0.19 | 0.03 |
| 892 | 0.68 | 0.01 | −0.23 | −0.55 | −0.10 | −0.10 | −0.10 | −0.13 |
| 917 | 0.30 | 0.07 | 0.18 | −0.53 | −0.57 | 0.21 | 0.28 | −0.03 |
| 933 | 0.68 | 0.17 | 0.14 | −0.05 | −0.47 | 0.10 | 0.06 | −0.04 |
| 1031 | −0.08 | 0.02 | −0.08 | −0.55 | 0.05 | 0.04 | 0.08 | −0.04 |
| 1054 | 0.64 | 0.02 | −0.19 | −0.36 | −0.22 | 0.03 | −0.05 | −0.11 |
| 1079 | 0.35 | 0.32 | 0.20 | −0.47 | −0.56 | 0.18 | 0.16 | −0.07 |
| 1095 | 0.72 | 0.15 | 0.24 | 0.02 | −0.31 | −0.25 | 0.14 | −0.07 |
| 1120 | 0.23 | −0.16 | −0.20 | −0.30 | −0.85 | −0.06 | 0.14 | 0.05 |
| 1136 | 0.12 | 0.02 | −0.50 | −0.10 | 0.14 | −0.77 | −0.19 | −0.03 |
| 1209 | 0.09 | −0.08 | −0.24 | −0.20 | 0.00 | −0.88 | −0.07 | 0.02 |
| 1216 | 0.91 | −0.14 | −0.03 | −0.10 | −0.01 | −0.15 | 0.02 | −0.11 |
| 1241 | 0.21 | 0.12 | 0.38 | −0.13 | −0.80 | −0.21 | 0.19 | 0.12 |
| 1257 | 0.08 | 0.55 | 0.27 | −0.10 | 0.07 | 0.24 | 0.58 | −0.01 |
| 1282 | −0.01 | 0.08 | −0.17 | −0.37 | −0.78 | −0.12 | −0.07 | −0.04 |
| 1298 | 0.15 | 0.46 | −0.01 | 0.33 | 0.61 | −0.21 | 0.02 | −0.11 |
| 1323 | 0.04 | −0.24 | −0.18 | −0.25 | −0.76 | 0.24 | 0.00 | 0.07 |
| 1339 | 0.03 | −0.17 | −0.22 | −0.33 | −0.74 | 0.36 | 0.07 | 0.02 |
| 1378 | 0.91 | −0.11 | −0.08 | 0.17 | −0.30 | −0.02 | 0.06 | −0.09 |
| 1393 | −0.25 | 0.10 | 0.20 | 0.23 | 0.14 | −0.17 | 0.47 | 0.06 |
| 1403 | 0.14 | 0.15 | 0.18 | −0.28 | −0.79 | 0.12 | 0.25 | −0.02 |
| 1419 | −0.17 | −0.22 | 0.87 | 0.27 | −0.05 | 0.19 | 0.09 | 0.08 |
| 1444 | −0.01 | 0.17 | 0.02 | 0.17 | −0.71 | −0.03 | −0.03 | 0.43 |
| 1460 | 0.11 | 0.67 | −0.35 | 0.20 | 0.49 | 0.18 | −0.01 | −0.18 |
| 1485 | −0.17 | 0.69 | 0.00 | −0.41 | 0.44 | −0.06 | −0.17 | −0.18 |
| 1501 | 0.11 | −0.14 | −0.24 | −0.39 | −0.70 | 0.35 | −0.03 | 0.12 |
| 1540 | 0.91 | −0.17 | −0.29 | 0.12 | 0.11 | −0.06 | 0.07 | 0.04 |
| 1555 | 0.06 | 0.04 | 0.44 | −0.13 | 0.26 | −0.05 | −0.16 | 0.35 |
| 1565 | 0.11 | 0.12 | 0.26 | −0.77 | 0.01 | 0.02 | 0.43 | 0.01 |
| 1581 | −0.54 | −0.47 | 0.59 | 0.07 | 0.02 | 0.00 | 0.03 | 0.24 |
| 1590 | 0.15 | −0.30 | 0.00 | 0.03 | 0.14 | −0.56 | 0.28 | 0.09 |
| 1606 | −0.19 | 0.82 | 0.21 | −0.03 | 0.15 | −0.23 | −0.11 | −0.22 |
| 1622 | 0.11 | 0.67 | −0.40 | 0.22 | 0.41 | 0.22 | 0.13 | −0.19 |
| 1647 | −0.41 | 0.73 | 0.22 | 0.03 | 0.22 | −0.34 | 0.13 | −0.16 |
| 1663 | −0.50 | 0.24 | −0.29 | 0.49 | 0.21 | −0.21 | −0.06 | −0.20 |
| 1688 | −0.22 | 0.26 | 0.17 | −0.74 | 0.00 | −0.21 | 0.28 | −0.31 |
| 1702 | 0.93 | −0.07 | −0.24 | 0.00 | −0.06 | −0.21 | 0.09 | −0.07 |
| 1704 | −0.09 | 0.90 | −0.06 | 0.14 | 0.00 | 0.11 | −0.22 | −0.24 |
| 1717 | 0.06 | −0.22 | 0.28 | −0.15 | 0.21 | −0.52 | 0.07 | −0.32 |
| 1743 | −0.67 | −0.49 | −0.02 | 0.09 | 0.40 | 0.06 | −0.15 | 0.16 |
| 1768 | −0.22 | 0.31 | −0.29 | −0.24 | 0.10 | −0.02 | 0.03 | 0.39 |
| 1784 | 0.08 | 0.11 | 0.06 | 0.10 | 0.07 | −0.01 | −0.28 | −0.80 |
| 1793 | 0.18 | −0.36 | −0.27 | 0.04 | −0.73 | 0.10 | 0.08 | 0.04 |
| 1809 | −0.59 | 0.20 | 0.05 | 0.50 | 0.06 | 0.03 | 0.06 | 0.02 |
| 1825 | −0.16 | −0.03 | −0.31 | −0.73 | −0.10 | 0.44 | −0.05 | 0.00 |
| 1850 | −0.10 | 0.74 | −0.25 | −0.31 | 0.02 | −0.03 | 0.22 | −0.24 |
| 1866 | −0.01 | 0.74 | −0.27 | 0.02 | 0.13 | 0.38 | −0.22 | −0.29 |
| 1905 | −0.26 | −0.42 | −0.41 | 0.33 | 0.34 | 0.03 | −0.55 | 0.00 |
| 1955 | −0.66 | −0.32 | −0.02 | 0.24 | 0.21 | −0.14 | 0.17 | 0.18 |
| 1971 | −0.12 | 0.55 | −0.26 | 0.01 | 0.01 | −0.40 | 0.45 | −0.03 |
| 1987 | 0.10 | −0.29 | −0.57 | 0.12 | −0.63 | 0.16 | −0.08 | 0.02 |
| 1996 | 0.05 | −0.14 | −0.14 | −0.42 | −0.75 | 0.22 | 0.21 | 0.04 |
| 2012 | 0.11 | 0.79 | −0.03 | 0.25 | 0.13 | −0.37 | −0.09 | −0.19 |
| 2028 | −0.57 | 0.02 | 0.26 | 0.44 | 0.25 | −0.25 | −0.22 | 0.14 |
| 2041 | −0.24 | 0.22 | 0.18 | 0.31 | 0.29 | −0.65 | −0.41 | 0.06 |
| 2067 | 0.01 | −0.32 | 0.06 | 0.45 | 0.45 | 0.05 | −0.64 | −0.02 |
| 2101 | −0.26 | −0.24 | 0.24 | 0.34 | −0.58 | −0.22 | 0.22 | 0.17 |
| 2117 | −0.01 | −0.03 | 0.20 | −0.54 | 0.10 | 0.07 | 0.12 | −0.04 |
| 2142 | 0.40 | −0.21 | −0.06 | 0.24 | 0.48 | 0.14 | 0.17 | 0.12 |
| 2158 | 0.18 | 0.03 | −0.17 | −0.08 | 0.06 | −0.73 | 0.35 | 0.03 |
| 2174 | −0.61 | −0.05 | 0.26 | 0.45 | 0.23 | −0.19 | −0.10 | 0.04 |
| 2229 | −0.02 | 0.46 | 0.12 | 0.21 | 0.18 | −0.42 | −0.52 | −0.27 |
| 2304 | 0.05 | −0.05 | 0.17 | −0.89 | 0.00 | −0.16 | 0.26 | 0.00 |
| 2320 | −0.28 | −0.31 | 0.09 | 0.10 | 0.10 | −0.42 | −0.08 | 0.12 |
| 2391 | 0.07 | 0.09 | −0.22 | −0.10 | 0.07 | −0.76 | 0.25 | 0.04 |
| 2393 | 0.10 | −0.02 | −0.21 | −0.14 | −0.01 | −0.06 | 0.08 | 0.02 |
| 1354 | −0.11 | 0.10 | −0.83 | −0.12 | −0.07 | −0.42 | 0.05 | 0.00 |
| 1362 | 0.40 | −0.12 | −0.05 | 0.10 | 0.25 | 0.09 | 0.14 | 0.01 |
| 1475 | 0.02 | −0.07 | 0.00 | −0.87 | −0.35 | −0.04 | 0.10 | −0.01 |
| 1500 | 0.17 | −0.28 | −0.46 | −0.46 | −0.35 | −0.37 | 0.11 | 0.08 |
| 1516 | 0.14 | 0.07 | −0.45 | −0.12 | 0.07 | −0.67 | 0.18 | 0.03 |
| 1541 | 0.02 | −0.18 | −0.89 | −0.16 | 0.00 | −0.21 | −0.11 | 0.00 |
| 1549 | −0.19 | −0.29 | 0.15 | 0.28 | 0.27 | 0.07 | 0.02 | 0.20 |
| 1557 | 0.02 | 0.23 | −0.74 | 0.21 | 0.08 | 0.40 | 0.23 | −0.02 |
| 1565 | −0.06 | −0.18 | −0.24 | 0.15 | 0.40 | −0.15 | 0.54 | 0.21 |
| 1637 | 0.10 | −0.09 | −0.01 | −0.90 | −0.31 | −0.09 | 0.12 | 0.01 |
| 1678 | −0.02 | 0.05 | −0.11 | −0.61 | −0.25 | −0.08 | 0.70 | 0.12 |
| 1703 | 0.43 | −0.09 | −0.53 | −0.12 | −0.41 | 0.03 | 0.23 | 0.03 |
| 1711 | 0.35 | 0.08 | 0.65 | −0.23 | −0.14 | 0.06 | −0.03 | −0.20 |
| 1719 | 0.41 | 0.06 | −0.66 | 0.21 | −0.44 | −0.16 | 0.30 | 0.06 |
| 1727 | −0.18 | 0.00 | −0.01 | −0.34 | 0.38 | 0.02 | 0.72 | 0.15 |
| 1744 | 0.07 | −0.02 | −0.22 | −0.53 | −0.02 | −0.28 | 0.14 | 0.03 |
| 1768 | 0.17 | 0.28 | −0.24 | 0.13 | 0.17 | 0.04 | 0.52 | 0.01 |
| 1791 | 0.00 | −0.12 | −0.78 | −0.21 | −0.03 | −0.51 | −0.14 | 0.01 |
| 1799 | −0.13 | −0.01 | −0.17 | −0.85 | −0.04 | 0.42 | 0.07 | −0.03 |
| 1840 | −0.12 | 0.14 | 0.18 | −0.57 | 0.08 | 0.24 | 0.49 | 0.04 |
| 1865 | 0.36 | −0.11 | −0.86 | −0.12 | 0.03 | 0.03 | 0.21 | 0.05 |
| 1873 | 0.02 | −0.01 | −0.13 | −0.34 | −0.17 | 0.12 | 0.80 | 0.11 |
| 1889 | −0.08 | 0.01 | −0.27 | −0.04 | −0.11 | 0.08 | 0.89 | 0.19 |
| 1906 | 0.38 | −0.17 | −0.31 | −0.59 | −0.14 | −0.13 | 0.45 | 0.09 |
| 1914 | 0.42 | −0.39 | −0.05 | −0.26 | −0.06 | −0.12 | 0.66 | 0.19 |
| 1930 | −0.35 | 0.01 | 0.04 | 0.63 | 0.52 | 0.19 | 0.01 | 0.08 |
| 1946 | −0.22 | −0.43 | 0.18 | 0.37 | 0.37 | 0.11 | 0.08 | 0.19 |
| 1947 | 0.17 | 0.09 | −0.26 | −0.55 | −0.38 | −0.25 | 0.35 | 0.02 |
| 2002 | 0.20 | 0.03 | 0.13 | −0.51 | −0.19 | −0.18 | 0.64 | 0.06 |
| 2010 | 0.00 | 0.10 | −0.83 | −0.25 | −0.01 | −0.13 | 0.19 | 0.01 |
| 2011 | 0.06 | −0.20 | 0.15 | −0.01 | −0.64 | 0.07 | 0.03 | 0.11 |
| 2035 | 0.22 | 0.08 | 0.00 | −0.63 | 0.08 | −0.23 | 0.41 | 0.04 |
| 2052 | 0.12 | −0.26 | 0.04 | −0.32 | −0.28 | −0.10 | 0.54 | 0.15 |
| 2068 | 0.10 | 0.02 | −0.46 | −0.73 | −0.01 | −0.16 | 0.20 | 0.02 |
| 2076 | −0.19 | 0.00 | 0.30 | 0.67 | 0.45 | 0.22 | −0.15 | 0.06 |
| 2092 | −0.24 | −0.27 | 0.48 | 0.56 | 0.12 | 0.01 | −0.02 | 0.45 |
| 2117 | 0.15 | −0.02 | 0.71 | −0.21 | −0.29 | −0.04 | 0.18 | 0.03 |
| 2133 | 0.03 | 0.01 | −0.31 | −0.69 | 0.17 | 0.06 | 0.34 | −0.01 |
| 2156 | 0.11 | −0.02 | −0.88 | −0.19 | −0.06 | −0.16 | 0.33 | 0.08 |
| 2157 | 0.01 | 0.80 | 0.06 | −0.08 | −0.17 | 0.15 | −0.12 | 0.37 |
| 2164 | 0.16 | −0.20 | −0.01 | 0.05 | 0.11 | −0.01 | 0.14 | 0.09 |
| 2221 | 0.03 | 0.14 | 0.12 | 0.20 | 0.29 | 0.05 | −0.13 | −0.84 |
| 2222 | −0.31 | 0.00 | 0.39 | 0.37 | 0.05 | 0.02 | −0.47 | 0.60 |
| 2230 | 0.37 | −0.09 | −0.70 | −0.15 | −0.42 | 0.02 | 0.19 | 0.06 |
| 2237 | −0.07 | −0.25 | 0.14 | 0.32 | 0.45 | 0.03 | 0.22 | 0.13 |
| 2238 | 0.24 | 0.05 | 0.12 | 0.01 | −0.42 | 0.08 | −0.28 | 0.60 |
| 2239 | 0.42 | −0.27 | 0.09 | 0.06 | −0.41 | 0.09 | 0.19 | 0.04 |
| 2253 | 0.00 | −0.21 | −0.04 | 0.26 | 0.37 | 0.13 | 0.05 | 0.08 |
| 2254 | −0.13 | −0.15 | 0.02 | 0.09 | 0.12 | 0.06 | −0.18 | 0.07 |
| 2263 | −0.16 | −0.25 | 0.60 | −0.05 | −0.20 | −0.09 | −0.16 | 0.50 |
| 2279 | 0.25 | 0.12 | 0.04 | −0.43 | −0.02 | 0.05 | 0.08 | −0.76 |
| 2280 | 0.05 | 0.20 | 0.16 | −0.50 | 0.24 | −0.14 | 0.16 | −0.60 |
| 2295 | −0.06 | −0.52 | 0.00 | 0.34 | 0.42 | 0.08 | 0.35 | 0.20 |
| 2321 | 0.02 | −0.18 | 0.09 | 0.15 | −0.75 | −0.01 | −0.02 | 0.06 |
| 2367 | −0.25 | 0.28 | 0.39 | 0.48 | 0.26 | 0.09 | −0.55 | −0.22 |
| 2368 | −0.27 | 0.35 | 0.24 | 0.19 | 0.20 | −0.11 | −0.23 | 0.57 |
| 2383 | −0.33 | −0.32 | 0.24 | 0.28 | 0.17 | 0.04 | 0.01 | 0.18 |
| 2384 | −0.29 | −0.35 | 0.16 | 0.33 | −0.32 | 0.00 | 0.06 | 0.11 |
| 2390 | −0.49 | −0.16 | 0.16 | 0.33 | 0.27 | 0.03 | −0.25 | 0.39 |
| 2400 | 0.21 | −0.17 | 0.28 | 0.04 | −0.44 | −0.14 | 0.10 | 0.00 |
| 2408 | −0.04 | 0.09 | 0.35 | 0.38 | −0.18 | 0.10 | −0.48 | 0.53 |
| 2425 | 0.07 | 0.09 | 0.34 | −0.45 | −0.53 | −0.06 | −0.02 | 0.51 |
| 2441 | −0.15 | 0.00 | 0.49 | 0.29 | 0.09 | 0.17 | −0.71 | −0.11 |
| 2447 | 0.25 | 0.03 | −0.72 | 0.07 | 0.06 | −0.16 | 0.31 | 0.05 |
| 2448 | 0.01 | 0.24 | −0.71 | 0.23 | 0.08 | 0.36 | 0.40 | −0.01 |
| 2482 | 0.00 | −0.13 | −0.05 | −0.76 | −0.44 | −0.07 | 0.15 | 0.01 |
| 2512 | 0.58 | −0.14 | −0.13 | 0.11 | 0.38 | 0.17 | 0.12 | 0.02 |
| 2521 | −0.08 | −0.31 | −0.29 | −0.09 | −0.27 | −0.21 | 0.20 | 0.11 |
| 2522 | 0.03 | 0.17 | −0.66 | 0.21 | −0.26 | 0.43 | 0.24 | 0.00 |
| 2528 | −0.08 | −0.17 | 0.03 | 0.11 | 0.14 | 0.06 | −0.13 | 0.07 |
| 2529 | 0.39 | −0.18 | 0.08 | 0.16 | 0.25 | 0.12 | 0.32 | 0.04 |
| 2544 | −0.12 | −0.15 | 0.01 | 0.10 | 0.12 | 0.07 | −0.18 | 0.07 |
| 2570 | −0.14 | 0.34 | 0.04 | −0.07 | 0.06 | 0.10 | 0.04 | −0.77 |
| 2571 | 0.15 | 0.13 | −0.04 | 0.06 | −0.37 | −0.39 | −0.16 | −0.69 |
| 2586 | 0.63 | −0.14 | 0.02 | 0.09 | 0.23 | 0.10 | 0.16 | 0.07 |
| 2587 | −0.32 | 0.16 | 0.27 | 0.18 | −0.06 | 0.01 | −0.52 | 0.65 |
| 2603 | 0.32 | −0.13 | 0.10 | 0.23 | −0.82 | 0.09 | 0.00 | 0.04 |
| 2644 | 0.12 | 0.13 | −0.11 | −0.33 | −0.14 | 0.08 | −0.10 | −0.86 |
| 2645 | 0.08 | 0.17 | 0.14 | 0.01 | 0.07 | 0.02 | −0.20 | −0.90 |
| 2683 | 0.25 | −0.28 | −0.28 | −0.21 | −0.46 | 0.08 | 0.16 | 0.04 |
| 2732 | −0.16 | 0.17 | 0.11 | 0.38 | 0.28 | 0.00 | −0.62 | −0.52 |
| 2733 | 0.02 | 0.38 | 0.13 | 0.04 | 0.28 | 0.04 | 0.07 | 0.39 |
| 2807 | −0.07 | 0.20 | 0.26 | 0.10 | −0.01 | −0.04 | −0.75 | −0.17 |
| 2878 | −0.13 | −0.06 | 0.04 | 0.05 | 0.34 | 0.15 | 0.26 | 0.04 |
| 2879 | 0.26 | −0.24 | 0.08 | 0.05 | 0.31 | 0.02 | 0.01 | 0.04 |
| 2886 | 0.11 | −0.13 | −0.45 | −0.46 | 0.02 | −0.30 | 0.01 | 0.01 |
| 2936 | −0.01 | 0.34 | 0.09 | 0.24 | 0.10 | 0.03 | −0.19 | −0.87 |
| 2953 | −0.35 | 0.06 | 0.25 | 0.24 | 0.14 | 0.05 | −0.53 | 0.44 |
| 3024 | −0.17 | 0.45 | −0.35 | 0.06 | −0.03 | −0.65 | 0.22 | 0.02 |
| 3025 | −0.40 | 0.43 | −0.05 | 0.22 | −0.02 | −0.01 | 0.20 | 0.04 |
| 3098 | −0.07 | 0.12 | 0.16 | 0.22 | 0.12 | 0.01 | −0.32 | −0.87 |
| 3099 | −0.28 | 0.13 | 0.03 | 0.24 | 0.14 | 0.18 | −0.68 | 0.48 |
| 3172 | 0.00 | 0.41 | 0.18 | 0.11 | 0.09 | 0.07 | −0.70 | 0.47 |
| 3390 | −0.41 | 0.26 | −0.01 | 0.18 | −0.02 | −0.01 | 0.06 | 0.05 |
| 3463 | −0.53 | −0.27 | 0.13 | 0.22 | 0.12 | −0.03 | −0.07 | 0.09 |
| Expl. Var | 15.087 | 13.057 | 17.101 | 19.989 | 17.715 | 9.359 | 14.429 | 12.390 |
| Prp. Totl | 0.093 | 0.080 | 0.105 | 0.123 | 0.109 | 0.057 | 0.089 | 0.076 |

TABLE 40
Raw Canonical Discriminant Function Coefficients, Eigenvalues, Means,
Tests of Significance of Squared Mahalanobis Distances and Classification
Matrix for acidic glycans from embryonic stem cells.

| | Wilks&apos; | Partial | F-remove | p-level | Toler. | 1-Toler. |
|---|---|---|---|---|---|---|
| 2092 | 0.000224 | 0.012148 | 203.3029 | 0.000016 | 0.014747 | 0.985253 |
| 2222 | 0.000179 | 0.015219 | 161.7677 | 0.000029 | 0.006831 | 0.993169 |
| 3463 | 0.000076 | 0.035969 | 67.0037 | 0.000245 | 0.011522 | 0.988478 |
| 2383 | 0.000102 | 0.026735 | 91.0094 | 0.000117 | 0.014976 | 0.985024 |
| 2482 | 0.000099 | 0.027361 | 88.8701 | 0.000124 | 0.013292 | 0.986708 |
| 2237 | 0.000080 | 0.034031 | 70.9618 | 0.000214 | 0.019852 | 0.980148 |
| 2408 | 0.000052 | 0.052279 | 45.3200 | 0.000625 | 0.006492 | 0.993508 |
| 1678 | 0.000040 | 0.068113 | 34.2039 | 0.001211 | 0.021660 | 0.978340 |
| 2368 | 0.000011 | 0.248367 | 7.5658 | 0.030742 | 0.147828 | 0.852172 |
| 1703 | 0.000010 | 0.273081 | 6.6548 | 0.038970 | 0.142395 | 0.857605 |

p-levels (Stem cell ACIDIC ES BM CB CD133 CD34 v03)

| | hESC | EB | st3 |
|---|---|---|---|
| hESC | | 0.000001 | 0.000000 |
| EB | 0.000001 | | 0.000005 |
| st3 | 0.000000 | 0.000005 | |

Classification Matrix

| | Percent | hESC | EB | st3 |
|---|---|---|---|---|
| hESC | 100.0000 | 4 | 0 | 0 |
| EB | 100.0000 | 0 | 7 | 0 |
| st3 | 100.0000 | 0 | 0 | 6 |
| Total | 100.0000 | 4 | 7 | 6 |

Chi-Square Tests with Successive Roots Removed
(Stem cell ACIDIC ES BM CB CD133 CD34 v03)

| | Eigen- | Canonicl | Wilks&apos; | Chi-Sqr. | df | p-level |
|---|---|---|---|---|---|---|
| 0 | 1926.162 | 0.999741 | 0.000003 | 121.7442 | 20 | 0.000000 |
| 1 | 189.829 | 0.997376 | 0.005240 | 49.8881 | 9 | 0.000000 |

Raw Coefficients (Stem cell ACIDIC ES BM CB CD133 CD34 v03)
for Canonical Variables

| | Root 1 | Root 2 |
|---|---|---|
| 2092 | 3.556 | 14.963 |
| 2222 | 3.870 | −5.504 |
| 3463 | 124.635 | −123.334 |
| 2383 | 3.237 | −19.567 |
| 2482 | −17.091 | −3.553 |
| 2237 | 9.232 | 2.593 |
| 2408 | 17.326 | 15.436 |
| 1678 | 8.774 | 3.763 |
| 2368 | 1.257 | 2.746 |
| 1703 | −3.675 | 0.679 |
| Constant | −41.386 | −8.778 |
| Eigenval | 1926.162 | 189.829 |
| Cum. Prop | 0.910 | 1.000 |

Means of Canonical Variables
(Stem cell ACIDIC ES BM CB CD133 CD34 v03)

| | Root 1 | Root 2 |
|---|---|---|
| hESC | 66.2050 | −8.7238 |
| EB | −4.0512 | 14.8899 |
| st3 | −39.4102 | −11.5557 |

TABLE 41
Raw Canonical Discriminant Function Coefficients, Eigenvalues, Means,
Tests of Significance of Squared Mahalanobis Distances and Classification
Matrix for combined neutral and acidic glycans from embryonic stem cells.
Raw Canonical Discriminant Function Coefficients (NEUTRAL
and ACIDIC) Sigma-restricted parameterization

| | Function | Function |
|---|---|---|
| Intercept | −171.07 | −109.808 |
| “730” | −20.64 | −3.629 |
| “1095” | −41.12 | 32.862 |
| “1540” | 19.51 | 1.698 |
| “1850” | −2.59 | 52.608 |
| “2174” | 312.41 | 12.862 |
| “1799” | 43.37 | 14.909 |
| “2092” | −7.76 | 8.021 |
| “2222” | 40.25 | 2.177 |
| “2230” | 27.50 | 8.883 |
| “2237” | 37.11 | 7.990 |
| “2280” | 11.20 | −3.048 |
| “2441” | −1.64 | 1.517 |
| “2587” | −19.79 | −12.728 |
| Eigenvalue | 33714.84 | 177.818 |
| Cum. Prop. | 0.99 | 1.000 |

Chi-Square Tests with Successive Roots Removed
Sigma-restricted parameterization

| | Eigen- | Canonicl | Wilk&apos; s | Chi-Sqr. | df | p-level |
|---|---|---|---|---|---|---|
| 0 | 33714.84 | 0.999985 | 0.000000 | 124.8967 | 26.00000 | 0.000000 |
| 1 | 177.82 | 0.997200 | 0.005592 | 41.4909 | 12.00000 | 0.000041 |

Class Means for Canonical Variables Sigma-restricted parameterization

| | hESC | EB | st3 |
|---|---|---|---|
| 1 | 296.9877 | −64.8879 | −122.289 |
| 2 | −3.2762 | 13.6745 | −13.769 |

Tests of Significance of Squared Mahalanobis Distances
F tests with 13 and 2. degrees of freedom Sigma-restricted parameterization

| | hESC | hESC | EB | EB | st3 | st3 |
|---|---|---|---|---|---|---|
| hESC | | | 3671.084 | 0.000272 | 4639.207 | 0.000216 |
| EB | 3671.084 | 0.000272 | | | 143.719 | 0.006930 |
| st3 | 4639.207 | 0.000216 | 143.719 | 0.006930 | | |

Classification Matrix Rows: Observed classifications
Columns: Predicted classifications

| | Percent | hESC | EB | st3 |
|---|---|---|---|---|
| hESC | 100.0000 | 4.000000 | 0.000000 | 0.000000 |
| EB | 100.0000 | 0.000000 | 7.000000 | 0.000000 |
| st3 | 100.0000 | 0.000000 | 0.000000 | 6.000000 |
| Total | 100.0000 | 4.000000 | 7.000000 | 6.000000 |

TABLE 42
m/z: neutral = [M + Na]⁺, sialylated = [M − H]⁻;
Composition: S = NeuAc, G = NeuGc, H = Hex,
N = HexNAc, F = dHex; ST (structure class): M = mannose-type,
H = hybrid-type, C = complex-type, O = other.

| | | | |
|---|---|---|---|
| Fig. | m/z | Composition | ST |

Neutral N-glycan fraction (FIG. 1.A)

| | | | |
|---|---|---|---|
| 609 | 609.21 | H1N2 | M |
| 771 | 771.26 | H2N2 | M |
| 917 | 917.32 | H2N2F1 | M |
| 933 | 933.31 | H3N2 | M |
| 1079 | 1079.38 | H3N2F1 | M |
| 1095 | 1095.37 | H4N2 | M |
| 1120 | 1120.40 | H2N3F1 | H |
| 1136 | 1136.40 | H3N3 | H |
| 1241 | 1241.43 | H4N2F1 | M |
| 1257 | 1257.42 | H5N2 | M |
| 1282 | 1282.45 | H3N3F1 | H |
| 1298 | 1298.45 | H4N3 | H |
| 1323 | 1323.48 | H2N4F1 | C |
| 1339 | 1339.48 | H3N4 | C |
| 1403 | 1403.48 | H5N2F1 | M |
| 1419 | 1419.48 | H6N2 | M |
| 1444 | 1444.51 | H4N3F1 | H |
| 1460 | 1460.50 | H5N3 | H |
| 1485 | 1485.53 | H3N4F1 | C |
| 1501 | 1501.53 | H4N4 | C |
| 1542 | 1542.56 | H3N5 | C |
| 1565 | 1565.53 | H6N2F1 | M |
| 1581 | 1581.53 | H7N2 | M |
| 1590 | 1590.57 | H4N3F2 | H |
| 1606 | 1606.56 | H5N3F1 | H |
| 1622 | 1622.56 | H6N3 | H |
| 1647 | 1647.59 | H4N4F1 | C |
| 1663 | 1663.58 | H5N4 | C |
| 1688 | 1688.61 | H3N5F1 | C |
| 1704 | 1704.61 | H4N5 | C |
| 1743 | 1743.58 | H8N2 | M |
| 1768 | 1768.61 | H6N3F1 | H |
| 1793 | 1793.64 | H4N4F2 | C |
| 1809 | 1809.64 | H5N4F1 | C |
| 1825 | 1825.63 | H6N4 | C |
| 1850 | 1850.67 | H4N5F3 | C |
| 1866 | 1866.66 | H5N5 | C |
| 1905 | 1905.63 | H9N2 | M |
| 1955 | 1955.70 | H5N4F2 | C |
| 1987 | 1987.69 | H7N4 | C |
| 1996 | 1996.72 | H4N5F2 | C |
| 2012 | 2012.72 | H5N5F1 | C |
| 2028 | 2028.71 | H6N5 | C |
| 2067 | 2067.69 | H10N2 | M |
| 2101 | 2101.76 | H5N4F3 | C |
| 2142 | 2142.78 | H4N5F3 | C |
| 2174 | 2174.77 | H6N5F1 | C |
| 2229 | 2229.74 | H11N2 | M |
| 2304 | 2304.84 | H5N5F3 | C |
| 2361 | 2361.87 | H5N6F2 | C |

Sialylated N-glycan fraction (FIG. 1.B)

| | | | |
|---|---|---|---|
| 1565 | 1565.55 | S1H4N3 | O |
| 1678 | 1678.60 | S2H2N3F1 | O |
| 1711 | 1711.61 | S1H4N3F1 | H |
| 1727 | 1727.60 | S1H5N3 | H |
| 1768 | 1768.57 | S1H4N4 | C |
| 1799 | 1799.62 | S2H4N2F1 | O |
| 1840 | 1840.65 | S2H3N3F1 | H |
| 1873 | 1873.66 | S1H5N3F1 | H |
| 1889 | 1889.65 | S1H6N3 | H |
| 1914 | 1914.68 | S1H4N4F1 | C |
| 1930 | 1930.68 | S1H5N4 | C |
| 1946 | 1946.67 | G1H5N4 | C |
| 1971 | 1971.71 | S1H4N5 | C |
| 2002 | 2002.70 | S2H4N3F1 | H |
| 2035 | 2035.71 | S1H6N3F1 | H |
| 2076 | 2076.74 | S1H5N4F1 | C |
| 2092 | 2092.73 | G1H5N4F1 | C |
| 2117 | 2117.76 | S1H4N5F1 | C |
| 2133 | 2133.76 | S1H5N5 | C |
| 2164 | 2164.75 | S2H5N3F1 | H |
| 2221 | 2221.78 | S2H5N4 | C |
| 2222 | 2222.80 | S1H5N4F2 | C |
| 2237 | 2237.77 | G1S1H5N4 | C |
| 2238 | 2238.79 | S1H6N4F1 | C |
| 2253 | 2253.76 | G2H5N4 | C |
| 2263 | 2263.82 | S1H4N5F2 | C |
| 2279 | 2279.82 | S1H5N5F1 | C |
| 2295 | 2295.81 | S1H6N5 | C |
| 2367 | 2367.83 | S2H5N4F1 | C |
| 2368 | 2368.85 | S1H5N4F3 | C |
| 2383 | 2383.83 | S2H6N4 | C |
| 2384 | 2384.85 | S1H6N4F2 | C |
| 2408 | 2408.86 | S2H4N5F1 | C |
| 2425 | 2425.87 | S1H5N5F2 | C |
| 2441 | 2441.87 | S1H6N5F1 | C |
| 2482 | 2482.90 | S1H5N6F2 | C |
| 2570 | 2570.91 | S2H5N5F1 | C |
| 2571 | 2571.93 | S1H5N5F3 | C |
| 2587 | 2587.93 | S1H6N5F2 | C |
| 2603 | 2603.92 | S1H7N5F1 | C |
| 2644 | 2644.95 | S1H6N6F1 | C |
| 2732 | 2732.97 | S2H6N5F1 | C |
| 2733 | 2733.99 | S1H6N5F3 | C |
| 2807 | 2807.00 | S1H7N6F1 | C |
| 2878 | 2878.00 | S3H6N5 | C |
| 2879 | 2879.02 | S2H6N5F2 | C |
| 2953 | 2953.06 | S1H7N6F2 | C |
| 3098 | 3098.10 | S2H7N6F1 | C |
| 3099 | 3099.12 | S1H7N6F3 | C |
| 3172 | 3172.13 | S1H8N7F1 | C |

TABLE 43
Comparison of lectin ligand profile in hESCs and MEFs

| Lectin | hESC | MEF |
|---|---|---|
| PSA | − | + |
| MAA | + | − |
| PNA | + | − |
| RCA | + | + |

+ present in cell surface
− not present in cell surface

TABLE 44
Lectins

| | FES29 | FES30 |
|---|---|---|
| PSA | − | − |
| LTA | +/− | − |
| UEA | + | − |
| MAA | + | + |
| SNA | (+/−) | (+/−) |
| RCA | + | + |
| PNA | + | + |
| PWA | + | + |
| STA | (+/−) | − |
| WFA | + | + |
| PHA-L | (+/−) | (+/−) |

TABLE 45
FACS

| | FES30 | FES61 |
|---|---|---|
| PSA | + | + |
| LTA | +/− | |
| UEA | + | + |
| MAA | + | + |
| SNA | + | |
| RCA | + | |
| PNA | + | + |
| PWA | +/− | − |
| STA | +/− | +/− |
| WFA | − | (+/−) |
| PHA-L | | |
| NPA | + | +/− |
| MBL | − | − |

TABLE 46
Antibodies

| | Immuno | FACS |
|---|---|---|
| GF281 | | − |
| GF285 | − | − |
| GF286 | +/− | + |
| GF287 | + | + |
| GBF372 | | − |
| GF373 | | − |
| anti-Le a | | − |
| GF368 | +/− | − |
| GF279 | + | + |
| GF280 | | − |
| GF284 | +/− | − |
| GF288 | +/− | − |
| GF289 | (+/−) | − |

TABLE 47
Antibodies

| | Immuno | FACS |
|---|---|---|
| GF403 | | − |
| GF418 | | − |
| anti-Le x | | − |
| anti-sialyl | | − |
| Le x | | |
| GF369 | +/− | − |
| GF370 | +/− | − |
| GF371 | | − |
| GF367 | +/− | + |
| GF401 | − | − |
| GF283 | +/− | |
| GF290 | (+/−) | |
| GF402 | +/− | |
| GF366 | − | |

TABLE 48

| | | | FES | | |
|---|---|---|---|---|---|
| Reagent | Target | FES 22 | 30 | mEF | % stain |
| FITC-PSA | α-Man | − | − | + | |
| FITC-RCA | β-Gal | + | − | +/− | |
| | (Galβ4GlcNAc) | | | | |
| FITC-PNA | β-Gal | + | + | − | |
| | (Galβ3GalNAc) | | | | |
| FITC-MAA | α2,3-sialyl-LN | + | + | − | |
| FITC-SNA | α2,6-sialyl-LN | + | n.d. | + | |
| FITC-PWA | I-antigen | + | + | n.d. | |
| FITC-STA | i-antigen | + | − | + | |
| FITC-WFA | β-GalNAc | + | + | − | |
| NeuGc-PAA- | NeuGc-lectin | + | + | + | |
| biotin | | | | | |
| anti-GM3(Gc) | NeuGcα3Galβ4Glc | + | + | + | |
| mAb | | | | | |
| FITC-LTA | α-Fuc | + | − | + | |
| FITC-UEA | α-Fuc | + | − | + | |
| mAb Lex | Lewis^{x} | + | n.d. | − | |
| mAb sLex | sialyl-Lewis^{x} | + | n.d. | − | |
| GF 279 Le c | Galβ3GlcNAc | | + | − | 95-100 |
| GF 283 Le b | | | + | − | 20-35 |
| GF 284 H Type 2 | | | + | − | 15-20 |
| GF 285 H Type 2 | | | − | + | 95-100 |
| GF 286 H Type 2 | | | + | − | 10-20 |
| GF 287 H Type 1 | | | + | − | 90-100 |
| GF 288 Globo-H | | | + | − | 20-35 |
| GF 289 Ley | | | − | + | 95-100 |
| GF 290 H Type 2 | | | + | − | 20-35 |

+, specific binding.
−, no specific binding.
n.d., not determined.
% of stain means approximate percentage of cell stained with a binder.

TABLE 49¹⁾

| | | | FES 21 | | FES 22 | | FES 29 | | FES 30 | | EB²⁾ |
|---|---|---|---|---|---|---|---|---|---|---|---|
| Affymetrix ID | Gene Bank ID | Gene | Det.³⁾ | Ch.⁴⁾ | Det. | Ch. | Det. | Ch. | Det. | Ch. | Det. |
| 206109_at | NM_000148.1 | FUT1 | P | I | P | I | P | I | P | I | A |
| 214088_s_at | AW080549 | FUT3 | M | NC | A | NC | A | NC | A | NC | A |
| 209892_at | AF305083.1 | FUT4 | P | I | P | I | P | I | P | I | A |
| 211225_at | U27330 | FUT5 | A | NC | A | NC | A | NC | A | NC | A |
| 211225_at | U27329.1 | FUT5 | A | NC | A | NC | A | NC | A | NC | A |
| 210399_x_at | U27336.1 | FUT6 | A | NC | A | NC | A | NC | A | NC | A |
| 211882_x_at | U27331.1 | FUT6(1) | A | NC | A | NC | A | NC | A | NC | A |
| 211885_x_at | U27332.1 | FUT6(2) | A | NC | A | NC | A | NC | A | NC | A |
| 211465_x_at | U27335.1 | FUT6(minor) | A | NC | A | NC | A | NC | A | NC | A |
| 210506_at | U11282.1 | FUT7 | A | NC | A | NC | A | NC | A | NC | A |
| 203988_s_at | NM_004480.1 | FUT8 | P | NC | P | NC | P | NC | P | NC | A |
| 207696_at | NM_006581.1 | FUT9 | A | NC | A | NC | A | NC | A | NC | A |
| 229203_at | NM_173593 | β4GalNAc-T3 | A | NC | A | NC | A | NC | A | NC | A |
| 200016_x_at | NM_002409 | MGAT3 | P | NC | P | D | P | D | P | D | P |
| 208058_s_at | NM_002409.2 | MGAT3 | A | NC | A | NC | A | NC | A | NC | A |
| 209764_at | AL022312 | β4GlcNAcT | A | NC | A | MD | A | MD | A | NC | A |
| 206435_at | NM_001478.2 | GALGT | A | NC | A | NC | A | NC | A | NC | A |
| 206720_at | NM_002410.2 | MGAT5 | A | NC | A | NC | A | NC | A | NC | A |
| 203102_s_at | NM_002408.2 | MGAT2 | P | I | P | NC | P | I | P | I | P |
| 201126_s_at | NM_002406.2 | MGAT1 | P | NC | P | NC | P | NC | P | NC | P |
| 219797_at | NM_012214.1 | GNT4a | A | NC | P | NC | A | NC | M | NC | A |
| 220189_s_at | NM_014275.1 | GNT4b | P | D | P | NC | P | NC | P | NC | P |
| 204856_at | AB049585 | β3GlcNAC-T3 | A | NC | A | NC | A | NC | A | NC | A |
| 225612_s_at | BE672260 | β3GlcNAc-T5 | P | D | P | D | P | D | P | D | P |
| 232337_at | XM_091928 | β3GlcNAc-T7 | P | NC | P | NC | P | NC | P | NC | A |
| 221240_s_at | NM_030765.1 | β3GlcNAc-T4 | P | NC | A | NC | A | NC | P | NC | A |
| 204856_at | NM_014256.1 | β3GnT3 | A | NC | A | NC | A | NC | A | NC | A |
| 205505_at | NM_001490.1 | β6GlcNAcT | P | I | P | NC | P | NC | A | NC | A |
| 203188_at | NM_006876.1 | i β3GlcNAcT | P | D | P | D | P | MD | P | NC | P |
| 211020_at | L19659.1 | I β6GlcNAcT | A | NC | M | NC | A | NC | A | NC | A |
| 214504_at | NM_020469.1 | A α3GalNAcT | A | NC | A | NC | A | NC | A | NC | A |
| 211812_s_at | AB050856.1 | globosideT | P | NC | A | NC | P | NC | P | NC | A |
| 221131_at | NM_016161.1 | α4GlCNAcT | M | NC | P | NC | P | NC | M | NC | A |
| 221935_s_at | | AER61 | P | I | P | I | P | I | P | I | A |
| 225689_at | | AGO61 | P | NC | P | NC | P | NC | P | NC | P |
| 210571_s_at | | CMAH | A | NC | A | NC | A | NC | A | NC | A |
| 205518_s_at | | CMAH | A | D | M | NC | A | D | A | NC | P |
| 213355_at | | ST3GAL6 | A | NC | A | NC | A | NC | A | NC | A |
| 211379_x_at | | β3GALT3 | P | D | P | D | P | NC | P | D | P |
| 218918_at | | MAN1C1 | P | NC | P | NC | P | NC | P | NC | P |
| 208450_at | | LGALS2 | A | NC | A | NC | A | NC | A | NC | A |
| 208949_s_at | | LGALS3 | P | D | P | D | P | D | P | D | P |

¹⁾Data reference: Skottman, H., et al. (2005).
²⁾EB, embryoid bodies used as reference in calculation of fold changes.
³⁾Det. (detection) codes: P, present; A, absent; M, medium.
⁴⁾Ch. (fold change) codes: I, increased; D, decreased; NC, no change.

TABLE 50
hESC-associated glycan groups revealed by statistical analysis.

| | | Preferred | Factors |
|---|---|---|---|
| Identification | Glycan class* | glycans^{#} | included^{§} |
| hESC-1 | Large high-mannose type and | H(6-9)N2 | 1-1, 6-1 |
| | glucosylated N-glycans | H(10-11)N2 | A3-3 |
| | | | A7-2 |
| hESC-2 | Small low-mannose type | H1N2 | 1-3 |
| | N-glycans | | |
| hESC-3 | Sialylated and neutral | H5N4F(1-2) | 1-1 |
| | biantennary- | | |
| | size complex-type N-glycans | S1H5N4F(0-1) | A4-1 |
| | | H5N4F1 | |
| hESC-4 | Large neutral or | H6N5F(0-1) | 1-2 |
| | monosialylated complex-type | S1H7N6F1 | A7-1 |
| | N-glycans | S(1-2)H6N5F1 | |
| | | S1H8N7F1 | |
| | | S1H7N6F3 | |
| hESC-5 | Neutral and sialylated small | H4N3 | 3-1 |
| | hybrid-type or monoantennary | S1H4N3F1 | A3-2 |
| | N-glycans | | |
| hESC-6 | Sialylated complex-type | S1H4N5F(1-2) | A3-1 |
| | N-glycans with N > H type | | |
| | non-reducing terminal | | |
| | HexNAc | | |
| hESC-7 | Complex-fucosylated | S1H6N5F2 | A8-1 |
| | complex-type N-glycans | S1H5N4F(2-3) | |

*Glycan class having shared molecular structure according to the present invention.
^{#}Preferred glycan signals for detection of the glycan group.
^{§}Described in detail under factor analysis specifications of the present invention with this Factor numbering.

TABLE 51
Differentiated cell-associated glycan groups in statistical analysis.

| | | Preferred | Factors |
|---|---|---|---|
| Identification | Glycan class* | glycans^{#} | included^{§} |
| Diff-1 | soluble HexNAc1-type | H(3-9)N1 | 1-1 |
| | glycans | | 5-1 |
| Diff-2 | non-fucosylated low- | H(2-4)N2 | 1-2 |
| | mannose type N-glycans | | |
| Diff-3 | fucosylated low-and high- | H(4-6)N2F1 | 3-2, 5-3 |
| | mannose type N-glycans | | A4-3 |
| | | | A5-2 |
| Diff-4 | small high-mannose type | H5N2 | 6-1 |
| | N-glycans | | A7-3 |
| Diff-5 | sialylated and neutral | H5N5(F0-1) | 2-2 |
| | complex-type N-glycans | H4N4(F0-2) | 3-4 |
| | with N═H type non- | H5N5F(1-3) | 4-2 |
| | reducing terminal HexNAc | S1H5N5 | 5-2 |
| | | H5N5F1P1 | A4-2 |
| | | S1H5N5F1A1 | A5-4 |
| | | S(1-2)H6N6F1 | A8-2 |
| Diff-6 | neutral and sialylated | H(5-6)N3(F0-1) | 2-3 |
| | hybrid-type and | H(2-3)N2F1 | 3-1 |
| | monoantennary N-glycans | H3N3 | 4-1 |
| | | H4N3F2 | A5-1 |
| | | H(2-4)N3F1 | A7-1 |
| | | S1H5N3F(0-1) | |
| Diff-7 | sulphated or phosphorylated | H3N4F1P1 | A3-1 |
| | N-glycans; preferably | S(0-2)H5N4F1P1 | |
| | including sulphate ester | S(0-1)H5N4P1 | |
| | | H4N3P1 | |
| | | S1H4N3F1P1 | |
| | | H4N4P1 | |
| | | S1H5N4F3P1 | |
| | | H6N5F1P1 | |
| | | H6N5F3P1 | |
| Diff-8 | small disialylated glycans, | S2H(2-4)N2F1 | A4-1 |
| | preferably including disialic | S2H(2-4)N3F1 | A7-2 |
| | acid | | |
| Diff-9 | multisialylated biantennary- | S2H5N4 | A8-1 |
| | size complex-type | S2H5N5F1 | |
| | N-glycans | | |
| Diff-10 | sialylated and neutral | H4N5 | 2-1 |
| | complex-type N-glycans | H4N5F(2-3) | 3-3 |
| | with N > H type non- | H3N4F(0-1) | A4-4 |
| | reducing terminal HexNAc | S1H5N6F2 | A5-3 |
| | | H3N5F1 | |
| Diff-11 | O-acetylated sialylated | S1H7N5F1A1 | A8-3 |
| | N-glycans | S1H6N4F1A1 | |

*, ^{#}, ^{§}See footnotes of the preceding Table.

1.-81. (canceled)

82. A method of evaluating the status of a human embryonic stem cell preparation comprising the step of detecting the presence of a glycan structure or a group of glycan structures in said preparation, wherein said glycan structure or a group of glycan structures is according to Formula T1 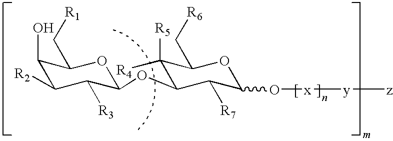
wherein X is linkage position
R₁, R₂, and R₆ are OH or glycosidically linked monosaccharide residue sialic acid, preferably Neu5Acα2 or Neu5Gc α2, most preferably Neu5Acα2 or
R₃, is OH or glycosidically linked monosaccharide residue Fucα1 (L-facose) or N-acetyl (N-acetamido, NCOCH₃);
R₄, is H, OH or glycosidically linked monosaccharide residue Fucα1 (L-fucose),
R₅ is OH, when R₄ is H, and R₅ is H, when R₄ is not H;
R7 is N-acetyl or OH;
X is natural oligosaccharide backbone structure from the cells, preferably N-glycan, O-glycan or glycolipid structure; or X is nothing, when n is 0,
Y is linker group preferably oxygen for O-glycans and O-linked terminal oligosaccharides and glycolipids and N for N-glycans or nothing when n is 0;
Z is a carrier structure, preferably natural carrier produced by the cells, such as protein or lipid, which is preferably a ceramide or branched glycan core structure on the carrier or H;
the arch indicates that the linkage from the galactopyranosyl is either to position 3 or to position 4 of the residue on the left and that the R4 structure is in the other position 4 or 3;
n is an integer 0 or 1, and m is an integer from 1 to 1000, preferably 1 to 100, and most preferably 1 to 10 (the number of the glycans on the carrier),
with the provisions that one of R2 and R3 is OH or R3 is N-acetyl,
R6 is OH, when the first residue on left is linked to position 4 of the residue on right:
X is not Galα4Galβ4Glc, (the core structure of SSEA-3 or 4) or R3 is fucosyl,
for the analysis of the status of stem cells and/or manipulation of the stem cells, and wherein said cell preparation is embryonic type stem cell preparation;
optionally, wherein the binder binds to the structure and additionally to at least one reducing end elongation epitope, preferably a monosaccharide epitope (replacing X and/or Y) according to Formula E1:
AxHex(NAc)ₙ, wherein A is anomeric structure alfa or beta, X is linkage position 2, 3, or 6; and
Hex is hexopyranosyl residue Gal, or Man, and n is integer being 0 or 1,
with the provisions that
when n is 1 then AxHexNAc is β4GalNAc or β6GalNAc,
when Hex is Man, then AxHex is β2Man, and
when Hex is Gal, then AxHex is β3Gal or β6Gal or α3Gal or α4Gal;
or
the binder epitope binds additionally to reducing end elongation epitope
Scr/Thr linked to reducing end GalNAcα-comprising structures or
βCer linked to Galβ4Glc comprising structures.

83. The method according to claim 82, wherein said binding agent recognizes structure according to Formula T8Ebeta
[Mα]ₘGalβ1-3/4[Nα]ₙGlcNAcβxHex(NAc)ₚ
wherein A is anomeric structure alfa or beta, X is linkage position 2, 3, or 6; and
wherein m, n and p are integers 0, or 1, independently
M and N are monosaccharide residues being
i) independently nothing (free hydroxyl groups at the positions) and/or
ii) SA which is sialic acid linked to 3-position of Gal or/and 6-position of GlcNAc and/or
iii) Fuc (L-fucose) residue linked to 2-position of Gal and/or 3 or 4 position of GlcNAc, when Gal is linked to the other position (4 or 3) of GlcNAc,
with the provision that m and n are 0 or 1, independently.
Hex is hexopyranosyl residue Gal, or Man,
with the provisions that when n is 1 then βxHexNAc is β6GalNAc,
when n is 0
then Hex is Man and βxHex is β2Man, or Hex is Gal and βxHex is β3Gal or β6Gal.

84. The method according to claim 82, wherein said binding agent recognizes type II Lactosamine based structures according to Formula T10Man:
[Mα]ₘGalβ1-4[Nα]ₙGlcNAcβ2Man,
wherein the variables are as described for Formula T8Ebeta in claim 83, wherein the structures can be such as Galβ4GlcNAcβ2Man, Galβ4(Fucα3)GlcNAcβ2Man, Fucα2Galβ4GlcNAcβ2Man, SAα6Galβ4GlcNAcβ2Man, and SAα3Galβ4GlcNAcβ2Man.

85. The method according to claim 82, wherein said binding agent recognizes type II Lactosamines according to Formula T10EGal(NAc):
[Mα]ₘGalβ1-4[Nα]ₙGlcNAcβ6Gal(NAc)ₚ
wherein the variables are as described for Formula T8Ebeta in claim 83, wherein the structures can be such as Galβ4GlcNAcβ6Gal, Galβ4GlcNAcβ6GalNAc, Gal4(Fucα3)GlcNAcβ6GalNAc, Fucα2Galβ4GlcNAcβ6GalNAc, SAα3/6Galβ4GlcNAcβ6GalNAc, and SAα3 Galβ4GlcNAcβ6GalNAc.

86. The method according to claim 82, wherein said binding agent recognizes type I Lactosamine based structures according to Formula T9E
[Mα]ₘGalβ1-3[Nα]ₘGlcNAcβ3Gal
wherein the structures can be such as Galβ3GlcNAcβ3Gal, Galβ3(Fucα4)βGlcNAcβ3Gal, and Fucα2Galβ3GlcNAcβ3Gal.

87. The method according to claim 82, wherein the elongated oligosaccharide structures are selected from the group consisting of (SAα3)₀ₒᵣ₁Galβ3/4(Fucα4/3)GlcNAc, Fucα2Galβ3GalNAcα/β, and Fucα2Galβ3(Fucα4)₀ₒᵣ₁GlcNAcβ.

88. The method according to claim 82, wherein the elongated oligosaccahride are selected from the group consisting of Galβ4Glc, Galβ3GlcNAc, Galβ3GalNAc, Galβ4GlcNAc, Galβ3 GlcNAcβ, Galβ3GalNAcβ/α, Galβ4GlcNAcβ, GalNAcβ4GlcNAc, SAα3Galβ4Glc, SAα3Galβ3GlcNAc, SAα3Galβ3GalNAc, SAα3Galβ4GlcNAc, SAα3Galβ3GlcNAcβ, SAα3Galβ3GalNAcβ/α, SAα3Galβ4GlcNAcβ, SAα6Galβ4Glc, SAα6Galβ4Glcβ, SAα6Galβ4GlcNAc, SAα6Galβ4GlcNAcβ, Galβ3(Fucα4)GlcNAc (Lewis a), Fucα2Galβ3GlcNAc (H-type 1), Fucα2Galβ3(Fucα4)GlcNAc (Lewis b), Galβ4GlcNAc (type 2 lactosamine based), Galβ4(Fucα3)GlcNAc (Lewis x), Fucα2Galβ4GlcNAc (H-type 2) and Fucα2Galβ4(Fucα3)GlcNAc (Lewis y).

89. The method according to claim 82, wherein the said binding agent binds to the same epitope than the antibodies selected from the group consisting of GF 287, GF 279, GF 288, GF 284, GF 283, GF 286, GF 290, GF 289, GF275, GF276, GF277, GF278, GF297, GF298, GF302, GF303, GF305, GF296, GF300, GF304, GF307, GF353, and GF354 and GF367.

90. The method according to claim 82, wherein the binder is used for sorting or selecting human embryonic (embryonal) stem cells from biological materials or samples including cell materials comprising other cell types.

91. The method according to claim 82, wherein the glycan structure is present in a O-glycan subglycome comprising O-Glycans with O-glycan core structure, or the glycan structure is present in a glycolipid subglycome comprising glycolipidss with glycolipid core structure and the glycans are releasable by glycosylceramidase or in a N-glycan subglycome comprising N-Glycans with N-glycan core structure and said N-Glycans being releasable from cells by N-glycosidase.

92. The method according to claim 82, wherein the presence or absence of cell surface glycomes of said cell preparation is detected.

93. The according to claim 82, method for identifying, characterizing, selecting or isolating stem cells in a population of mammalian cells which comprises using a binder or binding agent, said binder/binding agent binding to a glycan structure or glycan structures, wherein said structure
(i) exhibits expression on/in stem cells and an absence of expression or low expression in feeder cells, or differentiated cells;
(ii) exhibits absence of expression or low expression in stem cells and expression or high expression or mainly expressed in feeder cells or differentiated cells;
(iii) exhibits expression in subpopulations of stem cells; or (iv) exhibits expression in subpopulations of differentiated stem cells.

94. A cell population obtained by the method according to claim 90.

95. The method according to claim 82, wherein said cell preparation is evaluated/detected with regard to a contaminating structure in a cell population of said cell preparation, time dependent changes or a change in the status of the cell population by glycosylation analysis using mass spectrometric analysis of glycans in said cell preparation

96. A composition comprising glycan structure according to claim 82 derived from a stem cell and a binder that binds to said glycan structure.

97. The composition according to claim 99, wherein the composition is used in a method for identifying a selective stem cell binder to a glycan structure of claim 82, which comprises:
selecting a glycan structure exhibiting specific expression in/on stem cells and absence of expression in/on feeder cells and/or differentiated somatic cells; and
confirming the binding of the binder to the glycan structure in/on stem cells,
or, wherein the composition is used kit for enrichment and detection of stem cells within a specimen, comprising: at least one reagent comprising a binder to detect glycan structure according to claim 82; and instructions for performing stem cell enrichment using the reagent, optionally including means for performing stem cell enrichment or
wherein the composition is for isolation of cellular components from stem cells comprising the novel target/marker structures.

98. A method of evaluating the status of a embryonal type stem cell preparation comprising the step of detecting the presence of a glycan structure or a group of glycan structures in said preparation, wherein said glycan structure or a group of glycan structures is according to Formula T11
[M]ₘGalβ1-x[Nα]ₙHex(NAc)ₚ, wherein m, n and p are integers 0, or 1, independently
Hex is Gal or Glc, X is linkage position;
M and N are monosaccharide residues being independently nothing (free hydroxyl groups at the positions) and/or
SAα which is sialic acid linked to 3-position of Gal or/and 6-position of HexNAc
Galα linked to 3 or 4-position of Gal, or
GalNAcβ linked to 4-position of Gal and/or
Fuc (L-fucose) residue linked to 2-position of Gal
and/or 3 or 4 position of HexNAc, when Gal is linked to the other position (4 or 3),
and HexNAc is GlcNAc, or 3-position of Glc when Gal is linked to the other position (3), with the provision that sum of m and n is 2
preferably m and n are 0 or 1, independently, and
with the provision that when M is Galα then there is no sialic acid linked to Galβ1, and
n is 0 and preferably x is 4.
with the provision that when M is GalNAcβ, then there is no sialic acid α6-linked to Galβ1, but sialic acid can be linked to position 4, and n is 0 and x is 4.

99. A N-glycan core marker structure, wherein the disaccharide epitope is the Manβ4GlcNAc structure in the core structure of N-linked glycan according to Formula CGN:
[Manα3]ₙ₁(Manα6)ₙ₂Manβ4GlcNAcβ4(Fucα6)ₙ₃GlcNAcxR,
wherein n1, n2 and n3 are integers 0 or 1, independently indicating the presence or absence of the residues, and
wherein the non-reducing end terminal Manα3/Manα6-residues can be elongated to the complex type, especially biantennary structures or to mannose type (high-Man and/or low Man) or to hybrid type structures for the analysis of the status of stem cells and/or manipulation of the stem cells, wherein xR indicates reducing end structure of N-glycan linked to protein or petide such as βAsn or βAsn-peptide or βAsn-protein, or free reducing end of N-glycan or chemical derivative of the reducing produced,
and/or wherein Manα3/Manα6-residues are elongated to the complex type, especially biantennary structures and n3 is 1 and wherein the Manβ4GlcNAc-epitope comprises the GlcNAc substitution or substitutions for the analysis of human embryonic stem cells.

100. The method using N-glycan marker accoding to the claim 99, wherein the structure is a Mannose type glycan according to the formula M2 or a complex type N-glycan according to the Formula GNβ2:
and wherein the amount of at least one structure is altered by decrease or increase in stem cells during differentiation and the structure corresponds to the monosaccharide
HₙN₂Fₘ composition H wherein H is hexose, preferably Man or Glc or Gal, and N is N-acetylhexosamine, preferably GlcNAc, F is deoxyhexose preferably fucose, n is an integer from 1 to 11, and m is 0 or 1.

101. The method according to the claim 100, wherein the structure is associated with embryonal type stem cells in comparison to differentiated cells derived thereof or wherein the structure belongs to the group of
hESC-ii, being Large complex-type N-glycan, including H6N5, and H6N5F1;
or the structure belongs to the group of hESC-iii, being biantennary-size complex-type N-glycan, including H5N4F1, H5N4F2, and H5N4F3;
or the structure belongs to the group of hESC-iv, being complex-fucosylated N-glycan, including H5N4F2, H5N4F3, and H4N5F3;
or the structure belongs to the group of
hESC-vii, being monoantennary type N-glycan, including H4N3, and H4N3F1;
or structure belongs to the group of
hESC-viii, being terminal HexNAc N-glycan, including H4N5F3;
or the structure is associated with differentiated embryonal type stem cells derived from embryonal stem cells in comparison to embryonal type stem cells;
or the structure belongs to the group of Diff-iv, being terminal HexNAc N-glycan, including H5N6F2, H3N4, H3N5, H4N4F2, H4N5F2, H4N4, H4N5F1, H2N4F1, H3N5F1, and H3N4F1;
or the structure belongs to the group of Diff-vi, being terminal HexNAc monoantennary N-glycan, including H3N3, H3N3F1, and H2N3F1;
or the structure belongs to the group of Diff-vii, being H═N type terminal HexNAc N-glycan, including H5N5F1, H5N5, and H5N5F3;
or the structure belongs to the group of Diff-ix, being complex-fucosylated monoantennary type N-glycan, including H4N3F2;
or structure is a hybrid type N-glycan associated with differentiated embryonal type stem cells derived from embryonal stem cells in comparison to embryonal type stem cells;
or the structure belongs to the group of Diff-viii, being Elongated hybrid-type N-glycan, including H6N4, and H7N4;
or the structure belongs to the group of Diff-v, being Hybrid-type N-glycan, including H5N3F1, H5N3, H6N3F1, and H6N3.
